(19) United States
(12) Patent Application Publication
(10) Pub. No.: US 2025/0282854 A1
(43) Pub. Date: Sep. 11, 2025

(54) TAU BINDING COMPOUNDS

(71) Applicant: VOYAGER THERAPEUTICS, INC., Lexington, MA (US)

(72) Inventors: Wencheng Liu, Cambridge, MA (US); Todd Carter, Winchester, MA (US); Ishan Sanjeev Shah, Andover, MA (US); Christopher Joseph Murray, Poleglass (IE); James Bernard McClory, Ballynaskeagh (IE); Dinah Wen-Yee Sah, Hopkinton, MA (US)

(21) Appl. No.: 19/060,013

(22) Filed: Feb. 21, 2025

Related U.S. Application Data
(63) Continuation of application No. 18/734,892, filed on Jun. 5, 2024, now Pat. No. 12,291,565.
Application No. 18/734,892 is a continuation of application No. PCT/US2023/074239, filed on Sep. 14, 2023.
(60) Provisional application No. 63/406,924, filed on Sep. 15, 2022.
(60) Provisional application No. 63/448,913, filed on Feb. 28, 2023.

(51) Int. Cl. C07K 16/18; A61K 39/00; A61P 25/28

(52) U.S. Cl. CPC C07K 16/18; A61P 25/28; A61K 2039/505; A61K 2039/545; C07K 2317/24; C07K 2317/565; C12N 2750/14143

(57) ABSTRACT

The present disclosure provides anti-tau antibodies and vectorization thereof (e.g., into AAV particles). Also provided are methods of using anti-tau antibodies and/or AAV particles for prevention, treatment, and/or diagnosis of neurological indications.

### RELATED APPLICATIONS

This application is a Continuation of U.S. patent application Ser. No. 18/734,892, filed on Jun. 5, 2024, which is a Continuation of International Application No. PCT/US2023/074239, filed on Sep. 14, 2023, which claims priority to U.S. Provisional Application No. 63/406,924 filed on Sep. 15, 2022, and U.S. Provisional Application No. 63/448,913 filed on Feb. 28, 2023; the entire contents of each of which are hereby incorporated by reference in their entirety.

### SEQUENCE LISTING

The instant application contains a Sequence Listing which has been submitted electronically in XML format and is hereby incorporated by reference in its entirety. Said XML copy, created on Feb. 20, 2025, is named V2071-3005USCON2_SL.xml and is 1,438,465 bytes in size.

### FIELD OF THE DISCLOSURE

The present disclosure presents tau binding compounds and adeno-associated virus (AAV) particles comprising the same.

### BACKGROUND OF THE INVENTION

Tauopathies are a group of neurodegenerative diseases characterized by the dysfunction and/or aggregation of the microtubule associated protein tau. Tau is normally a soluble protein known to associate with microtubules based on the extent of its phosphorylation. Tau is considered an important component of intracellular trafficking processes, particularly in neuronal cells, given their unique and extended structure. Hyperphosphorylation of tau depresses its binding to microtubules and microtubule assembly activity. Further, hyperphosphorylation of tau renders it prone to misfolding and aggregation. In tauopathies, the tau becomes hyperphosphorylated, misfolds and aggregates as neurofibrillary tangles (NFT) of paired helical filaments (PHF), twisted ribbons or straight filaments. These NFT are largely considered indicative of impending neuronal cell death and thought to contribute to widespread neuronal cell loss, leading to a variety of behavioral and cognitive deficits.

A genetically defined tauopathy was described when mutations in the tau gene were shown to lead to an autosomal dominantly inherited tauopathy known as frontotemporal dementia and parkinsonism linked to chromosome 17 (FTDP-17). This provided evidence that changes in tau could lead to neurodegenerative changes in the brain. These molecules are considered to be more amyloidogenic, meaning they are more likely to become hyperphosphorylated and more likely to aggregate into NFT (Hutton, M. et al., 1998, Nature 393(6686):702-5).

Several approaches have been proposed for therapeutically interfering with progression of tau pathology and preventing the subsequent molecular and cellular consequences. Given that NFT are composed of hyperphosphorylated, misfolded and aggregated forms of tau, interference at each of these stages provides targets that can be pursued. Introducing agents that limit phosphorylation, block misfolding or prevent aggregation are promising strategies. It has also been suggested that introduction of anti-tau antibodies can prevent the trans-neuronal spread of tau pathology.

There remains a need for anti-tau antibodies for use in tauopathy treatment, diagnostics, and other applications. The present disclosure addresses this need with related compounds and methods described herein.

### SUMMARY OF THE INVENTION

The present disclosure pertains at least in part to compositions and methods for modulating the level of tau, e.g., aggregation and or distribution of tau, and/or delivery, e.g., vectorized delivery of an antibody that binds to tau, e.g., an anti-tau antibody, e.g., an anti-tau antibody described herein. In some embodiments, the level of tau, e.g., aggregation or distribution, is reduced or inhibited using an anti-tau antibody described herein or an isolated, e.g., recombinant, AAV particle comprising a viral genome encoding an anti-tau antibody, e.g., an anti-tau antibody described herein. In some embodiments, the degradation of tau is increased using an anti-tau antibody described herein or an isolated, e.g., recombinant, AAV particle comprising a viral genome encoding an anti-tau antibody, e.g., an anti-tau antibody described herein. Such inhibition and/or degradation can be useful in treating disorders related to expression of tau and/or neurological disorders, such as tauopathies.

Accordingly, in one aspect, the present disclosure provides an isolated, e.g., recombinant antibody that binds to tau, comprising a heavy chain variable region (VH) comprising one, two, or three of a heavy chain complementary determining region 1 (HC CDR1), a heavy chain complementary determining region 2 (HC CDR2), and/or a heavy chain complementary determining region 3 (HC CDR3) of any of the HC CDR sequences of Table 1 or 4; and/or a light chain variable region (VL) comprising one, two, or three of a light chain complementary determining region 1 (LC CDR1), a light chain complementary determining region 2 (LC CDR2), and/or a light chain complementary determining region 3 (LC CDR3) of any of the LC CDR sequences of Table 1 or 4.

In yet another aspect, the present disclosure provides an antibody that binds to human tau, which comprises a heavy chain variable region (VH) comprising a heavy chain complementary determining region 1 (HC CDR1), a heavy chain complementary determining region 2 (HC CDR2), and a heavy chain complementary determining region 3 (HC CDR3) and/or a light chain variable region (VL) comprising a light chain complementary determining region 1 (LC CDR1), a light chain complementary determining region 2 (LC CDR2), and a light chain complementary determining region 3 (LC CDR3), wherein (a) the HC CDR1, HC CDR2, HC CDR3, LC CDR1, LC CDR2, and LC CDR3 comprise the amino acid sequence of SEQ ID NO: 64, 1145, 1167, 1146, 529, and 571, respectively; (b) the HC CDR1, HC CDR2, HC CDR3, LC CDR1, LC CDR2, and LC CDR3 comprise the amino acid sequence of SEQ ID NO: 1144, 1145, 410, 1146, 529, and 571, respectively; (c) the HC CDR1, HC CDR2, HC CDR3, LC CDR1, LC CDR2, and LC CDR3 comprise the amino acid sequence of SEQ ID NO: 1165, SEQ ID NO: 1166, SEQ ID NO: 1167, SEQ ID NO: 473, RVS, and SEQ ID NO: 571, respectively; or (d) the HC CDR1, HC CDR2, HC CDR3, LC CDR1, LC CDR2, and LC CDR3 comprise the amino acid sequence of SEQ ID NO: 314, 341, 410, 1154, 529, and 571, respectively. In some embodiments, the antibody is a humanized antibody.

In yet another aspect, the present disclosure provides an antibody that binds to human tau, which comprises a heavy chain variable region (VH) comprising a heavy chain complementary determining region 1 (HC CDR1), a heavy chain complementary determining region 2 (HC CDR2), and a heavy chain complementary determining region 3 (HC CDR3) and/or a light chain variable region (VL) comprising a light chain complementary determining region 1 (LC CDR1), a light chain complementary determining region 2 (LC CDR2), and a light chain complementary determining region 3 (LC CDR3), wherein: (a) the HC CDR1, HC CDR2, HC CDR3, LC CDR1, LC CDR2, and LC CDR3 comprise the amino acid sequence of SEQ ID NO: 64, 1145, 1167, 1146, 529, and 571, respectively; (b) the HC CDR1, HC CDR2, HC CDR3, LC CDR1, LC CDR2, and LC CDR3 comprise the amino acid sequence of SEQ ID NO: 1144, 1145, 410, 1146, 529, and 571, respectively; (c) the HC CDR1, HC CDR2, HC CDR3, LC CDR1, LC CDR2, and LC CDR3 comprise the amino acid sequence of SEQ ID NO: 1165, SEQ ID NO: 1166, SEQ ID NO: 1167, SEQ ID NO: 473, RVS, and SEQ ID NO: 571, respectively; or (d) the HC CDR1, HC CDR2, HC CDR3, LC CDR1, LC CDR2, and LC CDR3 comprise the amino acid sequence of SEQ ID NO: 314, 341, 410, 1154, 529, and 571, respectively; and wherein: (i) the VH comprises an amino acid sequence comprising one, two, three, four, five, six, seven, eight, nine, or all of: an amino acid other than Q at position 5, P at position 7, T at position 9, L at position 11, V at position 12, N at position 19, L at position 20, K at position 67, A at position 68, and/or S at position 76, numbered according to SEQ ID NO: 21; and/or (ii) the VL comprises and amino acid sequence comprising one, two, or all of: an amino acid other than D at position 17, Q at position 18, and/or G at position 68, numbered according to SEQ ID NO: 93.

In yet another aspect, the present disclosure provides antibody that binds to human tau, which comprises a heavy chain variable region (VH) comprising a heavy chain complementary determining region 1 (HC CDR1), a heavy chain complementary determining region 2 (HC CDR2), and a heavy chain complementary determining region 3 (HC CDR3) and/or a light chain variable region (VL) comprising a light chain complementary determining region 1 (LC CDR1), a light chain complementary determining region 2 (LC CDR2), and a light chain complementary determining region 3 (LC CDR3), wherein: (a) the HC CDR1, HC CDR2, HC CDR3, LC CDR1, LC CDR2, and LC CDR3 comprise the amino acid sequence of SEQ ID NO: 64, 1145, 1167, 1146, 529, and 571, respectively; (b) the HC CDR1, HC CDR2, HC CDR3, LC CDR1, LC CDR2, and LC CDR3 comprise the amino acid sequence of SEQ ID NO: 1144, 1145, 410, 1146, 529, and 571, respectively; (c) the HC CDR1, HC CDR2, HC CDR3, LC CDR1, LC CDR2, and LC CDR3 comprise the amino acid sequence of SEQ ID NO: 1165, SEQ ID NO: 1166, SEQ ID NO: 1167, SEQ ID NO: 473, RVS, and SEQ ID NO: 571, respectively; or (d) the HC CDR1, HC CDR2, HC CDR3, LC CDR1, LC CDR2, and LC CDR3 comprise the amino acid sequence of SEQ ID NO: 314, 341, 410, 1154, 529, and 571, respectively; and wherein: (i) the VH comprises an amino acid sequence comprising one, two, three, four, five, six, seven, eight, or all of: a V at position 5, an S at position 7, an A at position 9, a V at position 11, a K at position 12, a K at position 19, a V at position 20, an R at position 67, and/or a V at position 68, numbered according to SEQ ID NO: 21; and/or (ii) the VL comprises and amino acid sequence comprising one, two, or all of: Q or E at position 17, P or R at position 18, and/or S at position 68, numbered according to SEQ ID NO: 93.

In yet another aspect, the present disclosure provides antibody that binds to human tau, which comprises a heavy chain variable region (VH) comprising a heavy chain complementary determining region 1 (HC CDR1), a heavy chain complementary determining region 2 (HC CDR2), and a heavy chain complementary determining region 3 (HC CDR3) and/or a light chain variable region (VL) comprising a light chain complementary determining region 1 (LC CDR1), a light chain complementary determining region 2 (LC CDR2), and a light chain complementary determining region 3 (LC CDR3), wherein: (a) the HC CDR1, HC CDR2, HC CDR3, LC CDR1, LC CDR2, and LC CDR3 comprise the amino acid sequence of SEQ ID NO: 64, 1145, 1167, 1146, 529, and 571, respectively; (b) the HC CDR1, HC CDR2, HC CDR3, LC CDR1, LC CDR2, and LC CDR3 comprise the amino acid sequence of SEQ ID NO: 1144, 1145, 410, 1146, 529, and 571, respectively; (c) the HC CDR1, HC CDR2, HC CDR3, LC CDR1, LC CDR2, and LC CDR3 comprise the amino acid sequence of SEQ ID NO: 1165, SEQ ID NO: 1166, SEQ ID NO: 1167, SEQ ID NO: 473, RVS, and SEQ ID NO: 571, respectively; or (d) the HC CDR1, HC CDR2, HC CDR3, LC CDR1, LC CDR2, and LC CDR3 comprise the amino acid sequence of SEQ ID NO: 314, 341, 410, 1154, 529, and 571, respectively; and wherein: (i) the VH comprises an amino acid sequence at least 86% (e.g., at least 90, 92, 95, 96, 97, 98, or 99%) identical to the amino acid sequence of any one of SEQ ID NOs: 67-71; and/or (ii) the VL comprises an amino acid sequence at least 91% (e.g., at least 92, 95, 96, 97, 98, or 99%) identical to the amino acid sequence of any one of SEQ ID NOs: 72-76.

In yet another aspect, the present disclosure provides an antibody that binds to human tau, which comprises a heavy chain variable region (VH) comprising a heavy chain complementary determining region 1 (HC CDR1), a heavy chain complementary determining region 2 (HC CDR2), and a heavy chain complementary determining region 3 (HC CDR3) and a light chain variable region (VL) comprising a light chain complementary determining region 1 (LC CDR1), a light chain complementary determining region 2 (LC CDR2), and a light chain complementary determining region 3 (LC CDR3), wherein: (a) the HC CDR1, HC CDR2, HC CDR3, LC CDR1, LC CDR2, and LC CDR3 comprise the amino acid sequence of SEQ ID NO: 64, 1145, 1167, 1146, 529, and 571, respectively; (b) the HC CDR1, HC CDR2, HC CDR3, LC CDR1, LC CDR2, and LC CDR3 comprise the amino acid sequence of SEQ ID NO: 1144, 1145, 410, 1146, 529, and 571, respectively; (c) the HC CDR1, HC CDR2, HC CDR3, LC CDR1, LC CDR2, and LC CDR3 comprise the amino acid sequence of SEQ ID NO: 1165, SEQ ID NO: 1166, SEQ ID NO: 1167, SEQ ID NO: 473, RVS, and SEQ ID NO: 571, respectively; or (d) the HC CDR1, HC CDR2, HC CDR3, LC CDR1, LC CDR2, and LC CDR3 comprise the amino acid sequence of SEQ ID NO: 314, 341, 410, 1154, 529, and 571, respectively; and wherein: (i) the VH comprises 1, 2, 3, 4, 5, 6, 7, 8, 9, 10, 11, 12, 13, 14, 15, 16, 17, 18, 19, 20, 21, or all of: V at position 5; S at position 7; A at position 9; V at position 11; K at position 12; S at position 14; A at position 16; K at position 19; V at position 20; R at position 38; Q at position 39; A at position 40; Q at position 43; R at position 67; V at position 68; I at position 71; R at position 72; D at position 73; T at position 74; T at position 76; T at position 84; and/or L at position 113, numbered according to SEQ ID NO: 21; and (ii) the VL comprises 1, 2, 3, 4, 5, 6, 7, 8, 9, 10, 11, 12, or all of: I at position 2; S at position 7; S at position 12; T at position 14; P at position 15; Q at position 17; P at position 18; Q at position 50; S at position 68; V at position 88; Y at position 92; Q at position 105; and/or V at position 109, numbered according to SEQ ID NO: 93.

In yet another aspect, the present disclosure provides n antibody that binds to human tau, which comprises a heavy chain variable region (VH) comprising a heavy chain complementary determining region 1 (HC CDR1), a heavy chain complementary determining region 2 (HC CDR2), and a heavy chain complementary determining region 3 (HC CDR3) and/or a light chain variable region (VL) comprising a light chain complementary determining region 1 (LC CDR1), a light chain complementary determining region 2 (LC CDR2), and a light chain complementary determining region 3 (LC CDR3), wherein: (a) the HC CDR1, HC CDR2, HC CDR3, LC CDR1, LC CDR2, and LC CDR3 comprise the amino acid sequence of SEQ ID NO: 64, 1145, 1167, 1146, 529, and 571, respectively; (b) the HC CDR1, HC CDR2, HC CDR3, LC CDR1, LC CDR2, and LC CDR3 comprise the amino acid sequence of SEQ ID NO: 1144, 1145, 410, 1146, 529, and 571, respectively; (c) the HC CDR1, HC CDR2, HC CDR3, LC CDR1, LC CDR2, and LC CDR3 comprise the amino acid sequence of SEQ ID NO: 1165, SEQ ID NO: 1166, SEQ ID NO: 1167, SEQ ID NO: 473, RVS, and SEQ ID NO: 571, respectively; or (d) the HC CDR1, HC CDR2, HC CDR3, LC CDR1, LC CDR2, and LC CDR3 comprise the amino acid sequence of SEQ ID NO: 314, 341, 410, 1154, 529, and 571, respectively; and wherein: (i) the VH comprises the amino acid sequence of SEQ ID NO: 69, or an amino acid sequence at least 86% (e.g., at least 90, 92, 95, 96, 97, 98, or 99/6) identical to the amino acid sequence of SEQ ID NO: 69; and/or (ii) the VL comprises the amino acid sequence of SEQ ID NO: 73, or an amino acid sequence at least 91% (e.g., at least 92, 95, 96, 97, 98, or 99%) identical to the amino acid sequence of SEQ ID NO: 73.

In yet another aspect, the present disclosure provides an antibody that binds to human tau, which comprises a heavy chain variable region (VH) comprising the amino acid sequence of SEQ ID NO: 69, and a light chain variable region (VL) comprising the amino acid sequence of SEQ ID NO: 73.

In yet another aspect, the present disclosure provides an antibody that binds to human tau, which comprises a heavy chain (HC) comprising the amino acid sequence of SEQ ID NO: 172, and a light chain (LC) comprising the amino acid sequence of SEQ ID NO: 176.

In yet another aspect, the present disclosure provides a nucleic acid encoding an antibody that binds to tau (e.g., an anti-tau antibody described herein).

In yet another aspect, the present disclosure provides vector encoding an antibody that binds to tau (e.g., an anti-tau antibody described herein).

In another aspect, the present disclosure provides a host cell comprising an antibody that binds to tau (e.g., an anti-tau antibody described herein) or a nucleic acid encoding an antibody that binds to tau (e.g., an anti-tau antibody described herein). In some embodiments, the host cell is a mammalian cell, an insect cell, or a bacterial cell.

In yet another aspect, the present disclosure provides a method of producing an antibody that binds to tau (e.g., an anti-tau antibody described herein). In some embodiments, the method comprising culturing a host cell comprising an anti-tau antibody described herein under conditions suitable for gene expression.

In yet another aspect, the present disclosure provides a viral genome comprising a promoter operably linked to a nucleic acid encoding an antibody that binds to tau (e.g., an anti-tau antibody described herein). In some embodiments, the viral genome further comprises an internal terminal repeat (ITR) sequence (e.g., an ITR region described herein), an enhancer (e.g., an enhancer described herein), an intron region (e.g., an intron region described herein) and/or an exon region (e.g., an exon region described herein), a poly A signal region (e.g., a poly A signal sequence described herein), and/or an encoded miR binding site.

In yet another aspect, the present disclosure provides an isolated, e.g., recombinant, AAV particle comprising a capsid protein and a viral genome comprising nucleic acid encoding an antibody that binds to tau (e.g., an anti-tau antibody described herein). In some embodiments, the capsid protein comprises an AAV capsid protein, e.g., a wild-type AAV capsid protein or a functional variant thereof. In some embodiments, the capsid protein comprises, or is chosen from, an AAV9 capsid protein, an AAV5 capsid protein, a VOY101 capsid protein, a PHP.N capsid protein, or a PHP.B capsid protein, or a functional variant thereof.

In yet another aspect, the present disclosure provides method of delivering an exogenous antibody that binds to tau (e.g., an anti-tau antibody described herein), to a subject. In some embodiments, the subject has or is diagnosed as having a neurological disorder, a tauopathy, and/or a disease associated with expression of tau. In some embodiments, the disease associated with Tau expression, the neurological disorder, or the tauopathy comprises AD, FTDP-17, FrLD, FTD, CTE, PSP, Down's syndrome, Pick's disease, CBD, Corticobasal syndrome, ALS, Prion diseases, CJD, Multiple system atrophy, mild cognitive impairment, Tangle-only dementia, or Progressive subcortical gliosis.

In yet another aspect, the present disclosure provides a method of treating a subject having or being diagnosed as having a neurological disorder, a tauopathy, and/or a disease associated with expression of tau. In some embodiments, the capsid protein comprises an AAV9 capsid protein or variant thereof. In some embodiments, the capsid protein comprises an AAV5 capsid protein or variant thereof. In some embodiments, the disease associated with Tau expression, the neurological disorder, or the tauopathy comprises AD, FTDP-17, FTLD, FTD, CTE, PSP, Down's syndrome, Pick's disease, CBD, Corticobasal syndrome, ALS, Prion diseases, CJD, Multiple system atrophy, mild cognitive impairment, Tangle-only dementia, or Progressive subcortical gliosis.

Those skilled in the art will recognize or be able to ascertain using no more than routine experimentation, many equivalents to the specific embodiments of the invention described herein. Such equivalents are intended to be encompassed by the following enumerated embodiment.

### Enumerated Embodiments

1. An antibody that binds to human tau, which comprises:
(a) a heavy chain variable region (VH) comprising a heavy chain complementarity determining region 1 (HC CDR1) comprising the amino acid sequence of SEQ ID NO: 64, a heavy chain complementarity determining region 2 (HC CDR2) comprising the amino acid sequence of SEQ ID NO: 1145, and a heavy chain complementarity determining region 3 (HC CDR3) comprising the amino acid sequence of SEQ ID NO: 1167; and a light chain variable region (VL) comprising a light chain complementarity determining region 1 (LC CDR1) comprising the amino acid sequence of SEQ ID NO: 1146, a light chain complementarity determining region 2 (LC CDR2) comprising the amino acid sequence of SEQ ID NO: 529, and a light chain complementarity determining region 3 (LC CDR3) comprising the amino acid sequence of SEQ ID NO: 571;
(b) a VH comprising a HC CDR1 comprising the amino acid sequence of SEQ ID NO: 1144, a HC CDR2 comprising the amino acid sequence of SEQ ID NO: 1145, and a HC CDR3 comprising the amino acid sequence of SEQ ID NO: 410; and a VL comprising a LC CDR1 comprising the amino acid sequence of SEQ ID NO: 1146, a LC CDR2 comprising the amino acid sequence of SEQ ID NO: 529, and a LC CDR3 comprising the amino acid sequence of SEQ ID NO: 571;
(c) a VH comprising a HC CDR1 comprising the amino acid sequence of SEQ ID NO: 1165, a HC CDR2 comprising the amino acid sequence of SEQ ID NO: 1166, and a HC CDR3 comprising the amino acid sequence of SEQ ID NO: 1167; and a VL comprising a LC CDR1 comprising the amino acid sequence of SEQ ID NO: 473, a LC CDR2 comprising the amino acid sequence of RVS, and a LC CDR3 comprising the amino acid sequence of SEQ ID NO: 571; or
(d) a VH comprising a HC CDR1 comprising the amino acid sequence of SEQ ID NO: 314, a HC CDR2 comprising the amino acid sequence of SEQ ID NO: 341, and a HC CDR3 comprising the amino acid sequence of SEQ ID NO: 410; and a VL comprising a LC CDR1 comprising the amino acid sequence of SEQ ID NO: 1154, a LC CDR2 comprising the amino acid sequence of SEQ ID NO: 529, and a LC CDR3 comprising the amino acid sequence of SEQ ID NO: 571;
optionally wherein, the antibody is a humanized antibody.
   2. An antibody that binds to human tau, which comprises:
(a) a heavy chain variable region (VH) comprising a heavy chain complementarity determining region 1 (HC CDR1) comprising the amino acid sequence of SEQ ID NO: 64, a heavy chain complementarity determining region 2 (HC CDR2) comprising the amino acid sequence of SEQ ID NO: 1145, and a heavy chain complementarity determining region 3 (HC CDR3) comprising the amino acid sequence of SEQ ID NO: 1167; and a light chain variable region (VL) comprising a light chain complementarity determining region 1 (LC CDR1) comprising the amino acid sequence of SEQ ID NO: 1146, a light chain complementarity determining region 2 (LC CDR2) comprising the amino acid sequence of SEQ ID NO: 529, and a light chain complementarity determining region 3 (LC CDR3) comprising the amino acid sequence of SEQ ID NO: 571;
(b) a VH comprising a HC CDR1 comprising the amino acid sequence of SEQ ID NO: 1144, a HC CDR2 comprising the amino acid sequence of SEQ ID NO: 1145, and a HC CDR3 comprising the amino acid sequence of SEQ ID NO: 410; and a VL comprising a LC CDR1 comprising the amino acid sequence of SEQ ID NO: 1146, a LC CDR2 comprising the amino acid sequence of SEQ ID NO: 529, and a LC CDR3 comprising the amino acid sequence of SEQ ID NO: 571;
(c) a VH comprising a HC CDR1 comprising the amino acid sequence of SEQ ID NO: 1165, a HC CDR2 comprising the amino acid sequence of SEQ ID NO: 1166, and a HC CDR3 comprising the amino acid sequence of SEQ ID NO: 1167; and a VL comprising a LC CDR1 comprising the amino acid sequence of SEQ ID NO: 473, a LC CDR2 comprising the amino acid sequence of RVS, and a LC CDR3 comprising the amino acid sequence of SEQ ID NO: 571; or
(d) a VH comprising a HC CDR1 comprising the amino acid sequence of SEQ ID NO: 314, a HC CDR2 comprising the amino acid sequence of SEQ ID NO: 341, and a HC CDR3 comprising the amino acid sequence of SEQ ID NO: 410; and a VL comprising a LC CDR1 comprising the amino acid sequence of SEQ ID NO: 1154, a LC CDR2 comprising the amino acid sequence of SEQ ID NO: 529, and a LC CDR3 comprising the amino acid sequence of SEQ ID NO: 571; and
wherein:
(i) the VH comprises an amino acid sequence comprising one, two, three, four, five, six, seven, eight, nine, or all of: an amino acid other than Q at position 5, P at position 7, T at position 9, L at position 11, V at position 12, N at position 19, L at position 20, K at position 67, A at position 68, and/or S at position 76, numbered according to SEQ ID NO: 21; and/or
(ii) the VL comprises and amino acid sequence comprising one, two, or all of: an amino acid other than D at position 17, Q at position 18, and/or G at position 68, numbered according to SEQ ID NO: 93.
   3. An antibody that binds to human tau, which comprises:
(a) a heavy chain variable region (VH) comprising a heavy chain complementarity determining region 1 (HC CDR1) comprising the amino acid sequence of SEQ ID NO: 64, a heavy chain complementarity determining region 2 (HC CDR2) comprising the amino acid sequence of SEQ ID NO: 1145, and a heavy chain complementarity determining region 3 (HC CDR3) comprising the amino acid sequence of SEQ ID NO: 1167; and a light chain variable region (VL) comprising a light chain complementarity determining region 1 (LC CDR1) comprising the amino acid sequence of SEQ ID NO: 1146, a light chain complementarity determining region 2 (LC CDR2) comprising the amino acid sequence of SEQ ID NO: 529, and a light chain complementarity determining region 3 (LC CDR3) comprising the amino acid sequence of SEQ ID NO: 571;
(b) a VH comprising a HC CDR1 comprising the amino acid sequence of SEQ ID NO: 1144, a HC CDR2 comprising the amino acid sequence of SEQ ID NO: 1145, and a HC CDR3 comprising the amino acid sequence of SEQ ID NO: 410; and a VL comprising a LC CDR1 comprising the amino acid sequence of SEQ ID NO: 1146, a LC CDR2 comprising the amino acid sequence of SEQ ID NO: 529, and a LC CDR3 comprising the amino acid sequence of SEQ ID NO: 571;
(c) a VH comprising a HC CDR1 comprising the amino acid sequence of SEQ ID NO: 1165, a HC CDR2 comprising the amino acid sequence of SEQ ID NO: 1166, and a HC CDR3 comprising the amino acid sequence of SEQ ID NO: 1167; and a VL comprising a LC CDR1 comprising the amino acid sequence of SEQ ID NO: 473, a LC CDR2 comprising the amino acid sequence of RVS, and a LC CDR3 comprising the amino acid sequence of SEQ ID NO: 571; or
(d) a VH comprising a HC CDR1 comprising the amino acid sequence of SEQ ID NO: 314, a HC CDR2 comprising the amino acid sequence of SEQ ID NO: 341, and a HC CDR3 comprising the amino acid sequence of SEQ ID NO: 410; and a VL comprising a LC CDR1 comprising the amino acid sequence of SEQ ID NO: 1154, a LC CDR2 comprising the amino acid sequence of SEQ ID NO: 529, and a LC CDR3 comprising the amino acid sequence of SEQ ID NO: 571; and
wherein:
(i) the VH comprises an amino acid sequence comprising one, two, three, four, five, six, seven, eight, or all of: a V at position 5, an S at position 7, an A at position 9, a V at position 11, a K at position 12, a K at position 19, a V at position 20, an R at position 67, and/or a V at position 68, numbered according to SEQ ID NO: 21; and/or
(ii) the VL comprises and amino acid sequence comprising one, two, or all of: Q or E at position 17, P or R at position 18, and/or S at position 68, numbered according to SEQ ID NO: 93.
   4. An antibody that binds to human tau, which comprises:
(a) a heavy chain variable region (VH) comprising a heavy chain complementarity determining region 1 (HC CDR1) comprising the amino acid sequence of SEQ ID NO: 64, a heavy chain complementarity determining region 2 (HC CDR2) comprising the amino acid sequence of SEQ ID NO: 1145, and a heavy chain complementarity determining region 3 (HC CDR3) comprising the amino acid sequence of SEQ ID NO: 1167; and a light chain variable region (VL) comprising a light chain complementarity determining region 1 (LC CDR1) comprising the amino acid sequence of SEQ ID NO: 1146, a light chain complementarity determining region 2 (LC CDR2) comprising the amino acid sequence of SEQ ID NO: 529, and a light chain complementarity determining region 3 (LC CDR3) comprising the amino acid sequence of SEQ ID NO: 571;
(b) a VH comprising a HC CDR1 comprising the amino acid sequence of SEQ ID NO: 1144, a HC CDR2 comprising the amino acid sequence of SEQ ID NO: 1145, and a HC CDR3 comprising the amino acid sequence of SEQ ID NO: 410; and a VL comprising a LC CDR1 comprising the amino acid sequence of SEQ ID NO: 1146, a LC CDR2 comprising the amino acid sequence of SEQ ID NO: 529, and a LC CDR3 comprising the amino acid sequence of SEQ ID NO: 571;
(c) a VH comprising a HC CDR1 comprising the amino acid sequence of SEQ ID NO: 1165, a HC CDR2 comprising the amino acid sequence of SEQ ID NO: 1166, and a HC CDR3 comprising the amino acid sequence of SEQ ID NO: 1167; and a VL comprising a LC CDR1 comprising the amino acid sequence of SEQ ID NO: 473, a LC CDR2 comprising the amino acid sequence of RVS, and a LC CDR3 comprising the amino acid sequence of SEQ ID NO: 571; or
(d) a VH comprising a HC CDR1 comprising the amino acid sequence of SEQ ID NO: 314, a HC CDR2 comprising the amino acid sequence of SEQ ID NO: 341, and a HC CDR3 comprising the amino acid sequence of SEQ ID NO: 410; and a VL comprising a LC CDR1 comprising the amino acid sequence of SEQ ID NO: 1154, a LC CDR2 comprising the amino acid sequence of SEQ ID NO: 529, and a LC CDR3 comprising the amino acid sequence of SEQ ID NO: 571; and
wherein:
(i) the VH comprises an amino acid sequence at least 86% (e.g., at least 90, 92, 95, 96, 97, 98, or 99%) identical to the amino acid sequence of any one of SEQ ID NOs: 67-71; and/or
(ii) the VL comprises an amino acid sequence at least 91% (e.g., at least 92, 95, 96, 97, 98, or 99%) identical to the amino acid sequence of any one of SEQ ID NOs: 72-76.
   5. The antibody of any one of embodiments 1-4, wherein the HC CDR1 comprises the amino acid sequence of SEQ ID NO: 64, the HC CDR2 comprises the amino acid sequence of SEQ ID NO: 1145, the HC CDR3 comprises the amino acid sequence of SEQ ID NO: 1167, the LC CDR1 comprises the amino acid sequence of SEQ ID NO: 1146, the LC CDR2 comprises the amino acid sequence of SEQ ID NO: 529, and the LC CDR3 comprises the amino acid sequence of SEQ ID NO: 571.
   6. The antibody of any one of embodiments 1-5, wherein the HC CDR1 comprises the amino acid sequence of SEQ ID NO: 1144, the HC CDR2 comprises the amino acid sequence of SEQ ID NO: 1145, the HC CDR3 comprises the amino acid sequence of SEQ ID NO: 410, the LC CDR1 comprises the amino acid sequence of SEQ ID NO: 1146, the LC CDR2 comprises the amino acid sequence of SEQ ID NO: 529, and the LC CDR3 comprises the amino acid sequence of SEQ ID NO: 571.
   7. The antibody of any one of embodiments 1-6, wherein the HC CDR1 comprises the amino acid sequence of SEQ ID NO: 1165, the HC CDR2 comprises the amino acid sequence of SEQ ID NO: 1166, the HC CDR3 comprises the amino acid sequence of SEQ ID NO: 1167, the LC CDR1 comprises the amino acid sequence of SEQ ID NO: 473, the LC CDR2 comprises the amino acid sequence of RVS, and the LC CDR3 comprises the amino acid sequence of SEQ ID NO: 571.
   8. The antibody of any one of embodiments 1-7, wherein the HC CDR1 comprises the amino acid sequence of SEQ ID NO: 314, the HC CDR2 comprises the amino acid sequence of SEQ ID NO: 341, the HC CDR3 comprises the amino acid sequence of SEQ ID NO: 410, the LC CDR1 comprises the amino acid sequence of SEQ ID NO: 1154, the LC CDR2 comprises the amino acid sequence of SEQ ID NO: 529, and the LC CDR3 comprises the amino acid sequence of SEQ ID NO: 571.
   9. The antibody of any one of embodiments 1-8, wherein the VH comprises an amino acid sequence comprising one, two, three, four, five, six, seven, eight, nine, or all of: an amino acid other than Q at position 5, P at position 7, T at position 9, L at position 11, V at position 12, N at position 19, L at position 20, K at position 67, A at position 68, and/or S at position 76, numbered according to SEQ ID NO: 21.
   10. The antibody of any one of embodiments 1-9, wherein the VH comprises:
(a) 1, 2, 3, 4, 5, 6, 7, 8, 9, 10, 11, 12, 13, 14, 15, 16, 17, 18, 19, 20, 21, or all of: an amino acid other than Q at position 5; an amino acid other than Q at position 6; an amino acid other than P at position 7; an amino acid other than T at position 9; an amino acid other than L at position 11; an amino acid other than V at position 12; an amino acid other than S at position 16; an amino acid other than N at position 19; an amino acid other than L at position 20; an amino acid other than I at position 48; an amino acid other than K at position 67; an amino acid other than A at position 68; an amino acid other than L at position 70; an amino acid other than S at position 76; an amino acid other than T at position 77; an amino acid other than V at position 79; an amino acid other than F at position 80; an amino acid other than I at position 81; an amino acid other than Q at position 82; an amino acid other than T at position 87; an amino acid other than S at position 91; and/or an amino acid other than S at position 113, numbered according to SEQ ID NO: 21;
(b) 1, 2, 3, 4, 5, 6, 7, 8, 9, 10, 11, 12, 13, 14, 15, 16, 17, 18, 19, 20, or all of: an amino acid other than Q at position 5; an amino acid other than P at position 7; an amino acid other than T at position 9; an amino acid other than L at position 11; an amino acid other than V at position 12; an amino acid other than S at position 16; an amino acid other than N at position 19; an amino acid other than L at position 20; an amino acid other than K at position 38; an amino acid other than K at position 67; an amino acid other than A at position 68; an amino acid other than L at position 70; an amino acid other than S at position 76; an amino acid other than T at position 77; an amino acid other than V at position 79; an amino acid other than F at position 80; an amino acid other than I at position 81; an amino acid other than Q at position 82; an amino acid other than T at position 87; an amino acid other than E at position 89; and/or an amino acid other than S at position 113, numbered according to SEQ ID NO: 21;
(c) 1, 2, 3, 4, 5, 6, 7, 8, 9, 10, 11, 12, 13, 14, 15, 16, 17, 18, 19, 20, 21, or all of: an amino acid other than Q at position 5; an amino acid other than P at position 7; an amino acid other than T at position 9; an amino acid other than L at position 11; an amino acid other than V at position 12; an amino acid other than P at position 14; an amino acid other than S at position 16; an amino acid other than N at position 19; an amino acid other than L at position 20; an amino acid other than K at position 38; an amino acid other than E at position 39; an amino acid other than R at position 40; an amino acid other than H at position 43; an amino acid other than K at position 67; an amino acid other than A at position 68; an amino acid other than T at position 71; an amino acid other than V at position 72; an amino acid other than H at position 73; an amino acid other than K at position 74; an amino acid other than S at position 76; an amino acid other than S at position 84; and/or an amino acid other than S at position 113, numbered according to SEQ ID NO: 21;
(d) 1, 2, 3, 4, 5, 6, 7, 8, 9, 10, 11, 12, 13, 14, 15, 16, 17, 18, 19, or all of: an amino acid other than Q at position 1; an amino acid other than Q at position 5; an amino acid other than P at position 7; an amino acid other than T at position 9; an amino acid other than L at position 11; an amino acid other than V at position 12; an amino acid other than N at position 19; an amino acid other than L at position 20; an amino acid other than K at position 67; an amino acid other than A at position 68; an amino acid other than L at position 70; an amino acid other than S at position 76; an amino acid other than V at position 79; an amino acid other than F at position 80; an amino acid other than I at position 81; an amino acid other than Q at position 82; an amino acid other than S at position 85; an amino acid other than E at position 89, an amino acid other than S at position 113; and/or an amino acid other than T at position 115, numbered according to SEQ ID NO: 21; or
(e) 1, 2, 3, 4, 5, 6, 7, 8, 9, 10, 11, 12, 13, 14, 15, 16 or all of: an amino acid other than Q at position 5; an amino acid other than P at position 7; an amino acid other than T at position 9; an amino acid other than L at position 11; an amino acid other than V at position 12; an amino acid other than S at position 16; an amino acid other than N at position 19; an amino acid other than L at position 20; an amino acid other than H at position 43; an amino acid other than I at position 48; an amino acid other than K at position 67; an amino acid other than A at position 68; an amino acid other than L at position 70; an amino acid other than S at position 76; an amino acid other than T at position 77; an amino acid other than T at position 87; and/or an amino acid other than S at position 91, numbered according to SEQ ID NO: 21.
   11. The antibody of any one of embodiments 1-10, wherein the VH comprises 1, 2, 3, 4, 5, 6, 7, 8, 9, 10, 11, 12, 13, 14, 15, 16, 17, 18, 19, 20, 21, or all of: an amino acid other than Q at position 5; an amino acid other than P at position 7; an amino acid other than T at position 9; an amino acid other than L at position 11; an amino acid other than V at position 12; an amino acid other than P at position 14; an amino acid other than S at position 16; an amino acid other than N at position 19; an amino acid other than L at position 20; an amino acid other than K at position 38; an amino acid other than E at position 39; an amino acid other than R at position 40; an amino acid other than H at position 43; an amino acid other than K at position 67; an amino acid other than A at position 68; an amino acid other than T at position 71; an amino acid other than V at position 72; an amino acid other than H at position 73; an amino acid other than K at position 74; an amino acid other than S at position 76; an amino acid other than S at position 84; and/or an amino acid other than S at position 113, numbered according to SEQ ID NO: 21.
   12. The antibody of any one of embodiments 1-10, wherein the VH comprises 1, 2, 3, 4, 5, 6, 7, 8, 9, 10, 11, 12, 13, 14, 15, 16, 17, 18, 19, 20, 21, or all of: an amino acid other than Q at position 5; an amino acid other than Q at position 6; an amino acid other than P at position 7; an amino acid other than T at position 9; an amino acid other than L at position 11; an amino acid other than V at position 12; an amino acid other than S at position 16; an amino acid other than N at position 19; an amino acid other than L at position 20; an amino acid other than I at position 48; an amino acid other than K at position 67; an amino acid other than A at position 68; an amino acid other than L at position 70; an amino acid other than S at position 76; an amino acid other than T at position 77; an amino acid other than V at position 79; an amino acid other than F at position 80; an amino acid other than I at position 81; an amino acid other than Q at position 82; an amino acid other than T at position 87; an amino acid other than S at position 91; and/or an amino acid other than S at position 113, numbered according to SEQ ID NO: 21.
   13. The antibody of any one of embodiments 1-10, wherein the VH comprises 1, 2, 3, 4, 5, 6, 7, 8, 9, 10, 11, 12, 13, 14, 15, 16, 17, 18, 19, or all of: an amino acid other than Q at position 1; an amino acid other than Q at position 5; an amino acid other than P at position 7; an amino acid other than T at position 9; an amino acid other than L at position 11; an amino acid other than V at position 12; an amino acid other than N at position 19; an amino acid other than L at position 20; an amino acid other than K at position 67; an amino acid other than A at position 68; an amino acid other than L at position 70; an amino acid other than S at position 76; an amino acid other than V at position 79; an amino acid other than F at position 80; an amino acid other than I at position 81; an amino acid other than Q at position 82; an amino acid other than S at position 85; an amino acid other than E at position 89, an amino acid other than S at position 113; and/or an amino acid other than T at position 115, numbered according to SEQ ID NO: 21.
   14. The antibody of any one of embodiments 1-10, wherein the VH comprises 1, 2, 3, 4, 5, 6, 7, 8, 9, 10, 11, 12, 13, 14, 15, 16 or all of: an amino acid other than Q at position 5; an amino acid other than P at position 7; an amino acid other than T at position 9; an amino acid other than L at position 11; an amino acid other than V at position 12; an amino acid other than S at position 16; an amino acid other than N at position 19; an amino acid other than L at position 20; an amino acid other than H at position 43; an amino acid other than I at position 48; an amino acid other than K at position 67; an amino acid other than A at position 68; an amino acid other than L at position 70; an amino acid other than S at position 76; an amino acid other than T at position 77; an amino acid other than T at position 87; and/or an amino acid other than S at position 91, numbered according to SEQ ID NO: 21.
   15. The antibody of any one of embodiments 1-14, wherein the VH comprises an amino acid sequence comprising one, two, three, four, five, six, seven, eight, or all of: a V at position 5, an S at position 7, an A at position 9, a V at position 11, a K at position 12, a K at position 19, a V at position 20, an R at position 67, and/or a V at position 68, numbered according to SEQ ID NO: 21
   16. The antibody of any one of embodiments 1-15, wherein the VH comprises:
(a) 1, 2, 3, 4, 5, 6, 7, 8, 9, 10, 11, 12, 13, 14, 15, 16, 17, 18, 19, 20, 21, or all of: V at position 5; E at position 6; S at position 7; A at position 9; V at position 11; K at position 12; A at position 16; K at position 19; V at position 20; M at position 48; R at position 67; V at position 68; I at position 70; A at position 76; S at position 77; A at position 79; Y at position 80; M at position 81; E at position 82; R at position 87; T at position 91; and/or T at position 113, numbered according to SEQ ID NO: 21;
(b) 1, 2, 3, 4, 5, 6, 7, 8, 9, 10, 11, 12, 13, 14, 15, 16, 17, 18, 19, 20, or all of: V at position 5; S at position 7; A at position 9; V at position 11; K at position 12; A at position 16; K at position 19; V at position 20; R at position 38; R at position 67; V at position 68; M at position 70; I at position 76; S at position 77; A at position 79; Y at position 80; M at position 81; E at position 82; R at position 87; D at position 89; and/or L at position 113, numbered according to SEQ ID NO: 21;
(c) 1, 2, 3, 4, 5, 6, 7, 8, 9, 10, 11, 12, 13, 14, 15, 16, 17, 18, 19, 20, 21, or all of: V at position 5; S at position 7; A at position 9; V at position 11; K at position 12; S at position 14; A at position 16; K at position 19; V at position 20; R at position 38; Q at position 39; A at position 40; Q at position 43; R at position 67; V at position 68; I at position 71; R at position 72; D at position 73; T at position 74; T at position 76; T at position 84; and/or L at position 113, numbered according to SEQ ID NO: 21;
(d) 1, 2, 3, 4, 5, 6, 7, 8, 9, 10, 11, 12, 13, 14, 15, 16, 17, 18, 19, or all of: E at position 1; V at position 5; S at position 7; A at position 9; V at position 11; K at position 12; K at position 19; V at position 20; R at position 67; V at position 68; M at position 70; I at position 76; A at position 79; Y at position 80; M at position 81; E at position 82; R at position 85; D at position 89, L at position 113; and/or S at position 115, numbered according to SEQ ID NO: 21; or
(e) 1, 2, 3, 4, 5, 6, 7, 8, 9, 10, 11, 12, 13, 14, 15, 16 or all of: V at position 5; S at position 7; A at position 9; V at position 11; K at position 12; A at position 16; K at position 19; V at position 20; Q at position 43; M at position 48; R at position 67; V at position 68; M at position 70; T at position 76; S at position 77; R at position 87; and/or T at position 91, numbered according to SEQ ID NO: 21.
   17. The antibody of any one of embodiments 1-11, 15, or 16, wherein the VH comprises 1, 2, 3, 4, 5, 6, 7, 8, 9, 10, 11, 12, 13, 14, 15, 16, 17, 18, 19, 20, 21, or all of: V at position 5; S at position 7; A at position 9; V at position 11; K at position 12; S at position 14; A at position 16; K at position 19; V at position 20; R at position 38; Q at position 39; A at position 40; Q at position 43; R at position 67; V at position 68; I at position 71; R at position 72; D at position 73; T at position 74; T at position 76; T at position 84; and/or L at position 113, numbered according to SEQ ID NO: 21.
   18. The antibody of any one of embodiments 1-10, 12, 15, or 16, wherein the VH comprises 1, 2, 3, 4, 5, 6, 7, 8, 9, 10, 11, 12, 13, 14, 15, 16, 17, 18, 19, 20, 21, or all of: V at position 5; E at position 6; S at position 7; A at position 9; V at position 11; K at position 12; A at position 16; K at position 19; V at position 20; M at position 48; R at position 67; V at position 68; I at position 70; A at position 76; S at position 77; A at position 79; Y at position 80; M at position 81; E at position 82; R at position 87; T at position 91; and/or T at position 113, numbered according to SEQ ID NO: 21.
   19. The antibody of any one of embodiments 1-10, 13, 15, or 16, wherein the VH comprises 1, 2, 3, 4, 5, 6, 7, 8, 9, 10, 11, 12, 13, 14, 15, 16, 17, 18, 19, or all of: E at position 1; V at position 5; S at position 7; A at position 9; V at position 11; K at position 12; K at position 19; V at position 20; R at position 67; V at position 68; M at position 70; I at position 76; A at position 79; Y at position 80; M at position 81; E at position 82; R at position 85; D at position 89, L at position 113; and/or S at position 115, numbered according to SEQ ID NO: 21.
   20. The antibody of any one of embodiments 1-10 or 14-16, wherein the VH comprises 1, 2, 3, 4, 5, 6, 7, 8, 9, 10, 11, 12, 13, 14, 15, 16 or all of: V at position 5; S at position 7; A at position 9; V at position 11; K at position 12; A at position 16; K at position 19; V at position 20; Q at position 43; M at position 48; R at position 67; V at position 68; M at position 70; T at position 76; S at position 77; R at position 87; and/or T at position 91, numbered according to SEQ ID NO: 21
   21. The antibody of any one of embodiments 1-20, wherein the VL comprises and amino acid sequence comprising one, two, or all of: an amino acid other than D at position 17, Q at position 18, and/or G at position 68, numbered according to SEQ ID NO: 93.
   22. The antibody of any one of embodiments 1-21, wherein the VL comprises:
(a) 1, 2, 3, 4, 5, 6, 7, 8, 9, 10, or all of: an amino acid other than T at position 7; an amino acid other than S at position 14; an amino acid other than D at position 17; an amino acid other than Q at position 18; an amino acid other than L at position 42; an amino acid other than K at position 44; an amino acid other than K at position 50; an amino acid other than G at position 68; an amino acid other than L at position 88; an amino acid other than F at position 92; and/or an amino acid other than G at position 105, numbered according to SEQ ID NO: 93;
(b) 1, 2, 3, 4, 5, 6, 7, 8, 9, 10, 11, 12, or all of: an amino acid other than V at position 2; an amino acid other than T at position 7; an amino acid other than P at position 12; an amino acid other than S at position 14; an amino acid other than L at position 15; an amino acid other than D at position 17; an amino acid other than Q at position 18; an amino acid other than K at position 50; an amino acid other than G at position 68; an amino acid other than L at position 88; an amino acid other than F at position 92; an amino acid other than G at position 105; and/or an amino acid other than L at position 109, numbered according to SEQ ID NO: 93;
(c) 1, 2, 3, 4, 5, 6, 7, 8, 9, 10, 11, 12, 13, or all of: an amino acid other than V at position 2; an amino acid other than L at position 11; an amino acid other than T at position 14; an amino acid other than D at position 17; an amino acid other than Q at position 18; an amino acid other than L at position 42; an amino acid other than K at position 44; an amino acid other than S at position 48; an amino acid other than K at position 50; an amino acid other than G at position 68; an amino acid other than S at position 72; an amino acid other than S at position 81; an amino acid other than L at position 88; and/or an amino acid other than G at position 105, numbered according to SEQ ID NO: 93;
(d) 1, 2, 3, 4, 5, 6, 7, 8, 9, 10, 11, or all of: an amino acid other than V at position 2; an amino acid other than V at position 3; an amino acid other than T at position 7; an amino acid other than S at position 14; an amino acid other than D at position 17; an amino acid other than Q at position 18; an amino acid other than L at position 42; an amino acid other than K at position 44; an amino acid other than K at position 50; an amino acid other than L at position 51; an amino acid other than G at position 68; and/or an amino acid other than L at position 88, numbered according to SEQ ID NO: 93; or
(e) 1, 2, 3, 4, 5, 6, 7, 8, 9, 10, 11, 12, 13, 14, 15, 16, 17, 18, 19, 20, 21, or all of: an amino acid other than D at position 1; an amino acid other than V at position 2; an amino acid other than M at position 4; an amino acid other than T at position 7; an amino acid other than L at position 9; an amino acid other than S at position 10; an amino acid other than P at position 12; an amino acid other than V at position 13; an amino acid other than L at position 15; an amino acid other than D at position 17; an amino acid other than Q at position 18; an amino acid other than S at position 20; an amino acid other than I at position 21; an amino acid other than S at position 48; an amino acid other than V at position 63; an amino acid other than G at position 68; an amino acid other than S at position 72; an amino acid other than K at position 79; an amino acid other than R at position 82; an amino acid other than V at position 83; an amino acid other than A at position 85; and/or an amino acid other than G at position 89, numbered according to SEQ ID NO: 93.
   23. The antibody of any one of embodiments 1-22, wherein the VL comprises 1, 2, 3, 4, 5, 6, 7, 8, 9, 10, 11, 12, or all of: an amino acid other than V at position 2; an amino acid other than T at position 7; an amino acid other than P at position 12; an amino acid other than S at position 14; an amino acid other than L at position 15; an amino acid other than D at position 17; an amino acid other than Q at position 18; an amino acid other than K at position 50; an amino acid other than G at position 68; an amino acid other than L at position 88; an amino acid other than F at position 92; an amino acid other than G at position 105; and/or an amino acid other than L at position 109, numbered according to SEQ ID NO: 93.
   24. The antibody of any one of embodiments 1-22, wherein the VL comprises 1, 2, 3, 4, 5, 6, 7, 8, 9, 10, or all of: an amino acid other than T at position 7; an amino acid other than S at position 14; an amino acid other than D at position 17; an amino acid other than Q at position 18; an amino acid other than L at position 42; an amino acid other than K at position 44; an amino acid other than K at position 50; an amino acid other than G at position 68; an amino acid other than L at position 88; an amino acid other than F at position 92; and/or an amino acid other than G at position 105, numbered according to SEQ ID NO: 93.
   25. The antibody of any one of embodiments 1-22, wherein the VL comprises 1, 2, 3, 4, 5, 6, 7, 8, 9, 10, 11, 12, 13, or all of: an amino acid other than V at position 2; an amino acid other than L at position 11; an amino acid other than T at position 14; an amino acid other than D at position 17; an amino acid other than Q at position 18; an amino acid other than L at position 42; an amino acid other than K at position 44; an amino acid other than S at position 48; an amino acid other than K at position 50; an amino acid other than G at position 68; an amino acid other than S at position 72; an amino acid other than S at position 81; an amino acid other than L at position 88; and/or an amino acid other than G at position 105, numbered according to SEQ ID NO: 93.
   26. The antibody of any one of embodiments 1-25, wherein the VL comprises and amino acid sequence comprising one, two, or all of: Q or E at position 17, P or R at position 18, and/or S at position 68, numbered according to SEQ ID NO: 93
   27. The antibody of any one of embodiments 1-26, wherein the VL comprises:
(a) 1, 2, 3, 4, 5, 6, 7, 8, 9, 10, or all of: S at position 7; T at position 14; Q at position 17; P at position 18; Q at position 42; R at position 44; R at position 50; S at position 68; V at position 88; Y at position 92; and/or Q at position 105, numbered according to SEQ ID NO: 93;
(b) 1, 2, 3, 4, 5, 6, 7, 8, 9, 10, 11, 12, or all of: I at position 2; S at position 7; S at position 12; T at position 14; P at position 15; Q at position 17; P at position 18; Q at position 50; S at position 68; V at position 88; Y at position 92; Q at position 105; and/or V at position 109, numbered according to SEQ ID NO: 93;
(c) 1, 2, 3, 4, 5, 6, 7, 8, 9, 10, 11, 12, 13, or all of: I at position 2; S at position 11; T at position 14; Q at position 17; P at position 18; Q at position 42; R at position 44; P at position 48; R at position 50; S at position 68; A at position 72; N at position 81; V at position 88; and/or Q at position 105, numbered according to SEQ ID NO: 93;
(d) 1, 2, 3, 4, 5, 6, 7, 8, 9, 10, 11, or all of: I at position 2; E at position 3; S at position 7; T at position 14; Q at position 17; P at position 18; Q at position 42; R at position 44; R at position 50; R at position 51; S at position 68; and/or V at position 88, numbered according to SEQ ID NO: 93; or
(e) 1, 2, 3, 4, 5, 6, 7, 8, 9, 10, 11, 12, 13, 14, 15, 16, 17, 18, 19, 20, 21, or all of: E at position 1; I at position 2; L at position 4; S at position 7; A at position 9; T at position 10; S at position 12; L at position 13; P at position 15; E at position 17; R at position 18; T at position 20; L at position 21; A at position 48; I at position 63; S at position 68; P at position 72; T at position 79; S at position 82; L at position 83; P at position 85; and/or A at position 89, numbered according to SEQ ID NO: 93.
   28. The antibody of any one of embodiments 1-23, 26, or 27, wherein the VL comprises 1, 2, 3, 4, 5, 6, 7, 8, 9, 10, 11, 12, or all of: I at position 2; S at position 7; S at position 12; T at position 14; P at position 15; Q at position 17; P at position 18; Q at position 50; S at position 68; V at position 88; Y at position 92; Q at position 105; and/or V at position 109, numbered according to SEQ ID NO: 93.
   29. The antibody of any one of embodiments 1-22, 24, 26, or 27, wherein the VL comprises 1, 2, 3, 4, 5, 6, 7, 8, 9, 10, or all of: S at position 7; T at position 14; Q at position 17; P at position 18; Q at position 42; R at position 44; R at position 50; S at position 68; V at position 88; Y at position 92; and/or Q at position 105, numbered according to SEQ ID NO: 93.
   30. The antibody of any one of embodiments 1-22 or 24-27, wherein the VL comprises 1, 2, 3, 4, 5, 6, 7, 8, 9, 10, 11, 12, 13, or all of: I at position 2; S at position 11; T at position 14; Q at position 17; P at position 18; Q at position 42; R at position 44; P at position 48; R at position 50; S at position 68; A at position 72; N at position 81; V at position 88; and/or Q at position 105, numbered according to SEQ ID NO: 93.
   31. The antibody of any one of embodiments 1-30, wherein:
(i) the VH comprises one, two, three, or four framework regions, e.g., one, two, three, or all of a FRH1, FRH2, FRH3, and/or FRH4; optionally wherein the VH comprises from N-terminus to C-terminus, FRH1-CDRH1-FRH2-CDRH2-FRH3-CDRH3-FRH4; and/or
(ii) the VL comprises one, two, three, or four framework regions, e.g., one, two, three, or all of a FRL1, FRL2, FRL3, and/or FRL4; optionally wherein the VL comprises from N terminus to C-terminus, FRL1-CDRL1-FRL2-CDRL2-FRL3-CDRL3-FRL4.
   32. The antibody of embodiment 31, wherein:
(a) (i) the FRH1 corresponds to positions 1-25 of a heavy chain variable region, numbered according to any one of SEQ ID NOs: 21 or 67-71; the FRH2 corresponds to positions 36-49, numbered according to any one of SEQ ID NOs: 21 or 67-71; the FRH3 corresponds to positions 67-96, numbered according to any one of SEQ ID NOs: 21 or 67-71; and/or the FRH4 corresponds to positions 108-118, numbered according to any one of SEQ ID NOs: 21 or 67-71; or
   (ii) the FRH1 corresponds to positions 1-30 of a heavy chain variable region, numbered according to any one of SEQ ID NOs: 21 or 67-71; the FRH2 corresponds to positions 36-49, numbered according to any one of SEQ ID NOs: 21 or 67-71; the FRH3 corresponds to positions 67-98, numbered according to any one of SEQ ID NOs: 21 or 67-71; and/or the FRH4 corresponds to positions 108-118, numbered according to any one of SEQ ID NOs: 21 or 67-71; and/or
(b) the FRL1 corresponds to positions 1-23 of a light chain variable region, numbered according to any one of SEQ ID NOs: 72-76 or 93; the FRL2 corresponds to positions 40-54 of a light chain variable region, numbered according to any one of SEQ ID NOs: 72-76 or 93; the FRL3 corresponds to positions 62-93 of a light chain variable region, numbered according to any one of SEQ ID NOs: 72-76 or 93; and/or the FRL4 corresponds to positions 103-112, numbered according to any one of SEQ ID NOs: 72-76 or 93.
   33. The antibody of any one of the preceding embodiments, wherein
(a) the VH comprises one, two, three, or all of:
   (i) a FRH1 comprising amino acids 1-25 or 1-30 of any one of SEQ ID NOs: 67-71 (optionally any one of SEQ ID NOs: 67 or 69-72); an amino acid sequence comprising one, two, three but no more than four modifications (e.g., substitutions, e.g., conservative substitutions) relative to amino acids 1-25 or 1-30 of any one of SEQ ID NOs: 67-71 (optionally any one of SEQ ID NOs: 67 or 69-72); or an amino acid sequence comprising one, two, three, but no more than four different amino acids relative to amino acids 1-25 or 1-30 of any one of SEQ ID NOs: 67-71 (optionally any one of SEQ ID NOs: 67 or 69-72);
   (ii) a FRH2 comprising amino acids 36-49 of any one of SEQ ID NOs: 67-71 (optionally any one of SEQ ID NOs: 67 or 69-72); an amino acid sequence comprising one, two, three but no more than four modifications (e.g., substitutions, e.g., conservative substitutions) relative to amino acids 36-49 of any one of SEQ ID NOs: 67-71 (optionally any one of SEQ ID NOs: 67 or 69-72); or an amino acid sequence comprising one, two, three, but no more than four different amino acids relative to amino acids 36-49 of any one of SEQ ID NOs: 67-71 (optionally any one of SEQ ID NOs: 67 or 69-72);
   (iii) a FRH3 comprising amino acids 67-96 or 67-98 of any one of SEQ ID NOs: 67-71 (optionally any one of SEQ ID NOs: 67 or 69-72); an amino acid sequence comprising one, two, three but no more than four modifications (e.g., substitutions, e.g., conservative substitutions) relative to amino acids 67-96 or 67-98 of any one of SEQ ID NOs: 67-71 (optionally any one of SEQ ID NOs: 67 or 69-72); or an amino acid sequence comprising one, two, three, but no more than four different amino acids relative to amino acids 67-96 or 67-98 of any one of SEQ ID NOs: 67-71 (optionally any one of SEQ ID NOs: 67 or 69-72); and/or
   (iv) a FRH4 comprising amino acids 108-118 of any one of SEQ ID NOs: 67-71 (optionally any one of SEQ ID NOs: 67 or 69-72); an amino acid sequence comprising one, two, three but no more than four modifications (e.g., substitutions, e.g., conservative substitutions) relative to amino acids 108-118 of any one of SEQ ID NOs: 67-71 (optionally any one of SEQ ID NOs: 67 or 69-72); or an amino acid sequence comprising one, two, three, but no more than four different amino acids relative to amino acids 108-118 of any one of SEQ ID NOs: 67-71 (optionally any one of SEQ ID NOs: 67 or 69-72); and/or
(b) the VL comprises one, two, three or all of:
   (i) a FRL1 comprising amino acids 1-23 of any one of SEQ ID NOs: 72-76 (optionally any one of SEQ ID NOs: 72-74); an amino acid sequence comprising one, two, three but no more than four modifications (e.g., substitutions, e.g., conservative substitutions) relative to amino acids 1-23 of any one of SEQ ID NOs: 72-76 (optionally any one of SEQ ID NOs: 72-74); or an amino acid sequence comprising one, two, three but no more than four different amino acids relative to amino acids 1-23 of any one of SEQ ID NOs: 72-76 (optionally any one of SEQ ID NOs: 72-74);
   (ii) a FRL2 comprising amino acids 40-54 of any one of SEQ ID NOs: 72-76 (optionally any one of SEQ ID NOs: 72-74); an amino acid sequence comprising one, two, three but no more than four modifications (e.g., substitutions, e.g., conservative substitutions) relative to amino acids 40-54 of any one of SEQ ID NOs: 72-76 (optionally any one of SEQ ID NOs: 72-74); or an amino acid sequence comprising one, two, three but no more than four different amino acids relative to amino acids 40-54 of any one of SEQ ID NOs: 72-76 (optionally any one of SEQ ID NOs: 72-74);
   (iii) a FRL3 comprising amino acids 62-93 of any one of SEQ ID NOs: 72-76 (optionally any one of SEQ ID NOs: 72-74); an amino acid sequence comprising one, two, three but no more than four modifications (e.g., substitutions, e.g., conservative substitutions) relative to amino acids 62-93 of any one of SEQ ID NOs: 72-76 (optionally any one of SEQ ID NOs: 72-74); or an amino acid sequence comprising one, two, three but no more than four different amino acids relative to amino acids 62-93 of any one of SEQ ID NOs: 72-76 (optionally any one of SEQ ID NOs: 72-74); and/or
   (iv) a FRL4 comprising amino acids 103-112 of any one of SEQ ID NOs: 72-76 (optionally any one of SEQ ID NOs: 72-74); an amino acid sequence comprising one, two, three but no more than four modifications (e.g., substitutions, e.g., conservative substitutions) relative to amino acids 103-112 of any one of SEQ ID NOs: 72-76 (optionally any one of SEQ ID NOs: 72-74); or an amino acid sequence comprising one, two, three but no more than four different amino acids relative to amino acids 103-112 of any one of SEQ ID NOs: 72-76 (optionally any one of SEQ ID NOs: 72-74).
      34. The antibody of any one of embodiments 1-33, wherein the:
(i) the VH does not comprise one, two, three, or all of a FRH1 comprising amino acids 1-25 or 1-30 of SEQ ID NO: 21; a FRH2 comprising amino acids 36-49 of SEQ ID NO: 21; an FRH3 comprising amino acids 67-96 or 67-98 of SEQ ID NO: 21; and/or a FRH4 comprising amino acids 108-118 of SEQ ID NO: 21; and/or
(ii) the VL does not comprise one, two, three, or all of a FRL1 comprising amino acids 1-23 of SEQ ID NO: 93; a FRL2 comprising amino acids 40-54 of SEQ ID NO: 93; an FRL3 comprising amino acids 62-93 of SEQ ID NO: 93; and/or a FRL4 comprising amino acids 103-112 of SEQ ID NO: 93.
   35. The antibody of any one of embodiments 1-34, wherein VH comprises the amino acid sequence of any one of SEQ ID NOs: 67-71; or an amino acid sequence at least 86% (e.g., at least 90, 92, 95, 96, 97, 98, or 99%) identical to the amino acid sequence of any one of SEQ ID NOs: 67-71.
   36. The antibody of any one of embodiments 1-35, wherein the VH comprises an amino acid sequence comprising at least one, two, three, four, or five but no more than 16 modifications, e.g., substitutions (e.g., conservative substitutions) relative to the amino acid sequence of any one of SEQ ID NOs: 67-71.
   37. The antibody of any one of embodiments 1-36, wherein the VH comprises an amino acid sequence comprising at least one, two, three, four, or five but no more than 16 different amino acids relative to the amino acid sequence of any one of SEQ ID NOs: 67-71.
   38. The antibody of any one of embodiments 1-11, 15-17, or 21-37, wherein VH comprises the amino acid sequence of SEQ ID NO: 69; or an amino acid sequence at least 86% (e.g., at least 90, 92, 95, 96, 97, 98, or 99%) identical to the amino acid sequence of SEQ ID NO: 69.
   39. The antibody of any one of embodiments 1-11, 15-17, or 21-38, wherein the VH comprises an amino acid sequence comprising at least one, two, three, four, or five but no more than 16 modifications, e.g., substitutions (e.g., conservative substitutions) relative to the amino acid sequence of SEQ ID NO: 69.
   40. The antibody of any one of embodiments 1-11, 15-17, or 21-39, wherein the VH comprises an amino acid sequence comprising at least one, two, three, four, or five but no more than 16 different amino acids relative to the amino acid sequence of SEQ ID NO: 69.
   41. The antibody of any one of embodiments 1-10, 12, 15, 16, 18 or 21-37, wherein VH comprises:
(i) the amino acid sequence of SEQ ID NO: 67; or an amino acid sequence at least 86% (e.g., at least 90, 92, 95, 96, 97, 98, or 99%) identical to the amino acid sequence of SEQ ID NO: 67;
(ii) an amino acid sequence comprising at least one, two, three, four, or five but no more than 16 modifications, e.g., substitutions (e.g., conservative substitutions) relative to the amino acid sequence of SEQ ID NO: 67; or
(iii) an amino acid sequence comprising at least one, two, three, four, or five but no more than 16 different amino acids relative to the amino acid sequence of SEQ ID NO: 67.
   42. The antibody of any one of embodiments 1-10, 13, 15, 16, 19, or 21-37, wherein VH comprises:
(i) the amino acid sequence of SEQ ID NO: 70; or an amino acid sequence at least 86% (e.g., at least 90, 92, 95, 96, 97, 98, or 99%) identical to the amino acid sequence of SEQ ID NO: 70;
(ii) an amino acid sequence comprising at least one, two, three, four, or five but no more than 16 modifications, e.g., substitutions (e.g., conservative substitutions) relative to the amino acid sequence of SEQ ID NO: 70; or
(iii) an amino acid sequence comprising at least one, two, three, four, or five but no more than 16 different amino acids relative to the amino acid sequence of SEQ ID NO: 70.
   43. The antibody of any one of embodiments 1-10, 15, 16, or 20-37, wherein VH comprises:
(i) the amino acid sequence of SEQ ID NO: 71; or an amino acid sequence at least 86% (e.g., at least 90, 92, 95, 96, 97, 98, or 99%) identical to the amino acid sequence of SEQ ID NO: 71;
(ii) an amino acid sequence comprising at least one, two, three, four, or five but no more than 16 modifications, e.g., substitutions (e.g., conservative substitutions) relative to the amino acid sequence of SEQ ID NO: 71; or
(iii) an amino acid sequence comprising at least one, two, three, four, or five but no more than 16 different amino acids relative to the amino acid sequence of SEQ ID NO: 71.
   44. The antibody of any one of embodiments 1-43, wherein VL comprises the amino acid sequence of any one of SEQ ID NOs: 72-76; or an amino acid sequence at least 91% (e.g., at least 92, 95, 96, 97, 98, or 99%) identical to the amino acid sequence of any one of SEQ ID NOs: 72-76.
   45. The antibody of any one of embodiments 1-44, wherein the VL comprises an amino acid sequence comprising at least one, two, three, four, or five but no more than 10 modifications, e.g., substitutions (e.g., conservative substitutions) relative to the amino acid sequence of any one of SEQ ID NOs: 72-76.
   46. The antibody of any one of embodiments 1-45, wherein the VL comprises an amino acid sequence comprising at least one, two, three, four, or five but no more than 10 different amino acids relative to the amino acid sequence of any one of SEQ ID NOs: 72-76.
   47. The antibody of any one of embodiments 1-23, 26-28, or 31-46, wherein VL comprises the amino acid sequence of SEQ ID NO: 73; or an amino acid sequence at least 91% (e.g., at least 92, 95, 96, 97, 98, or 99%) identical to the amino acid sequence of SEQ ID NO: 73.
   48. The antibody of any one of embodiments 1-23, 26-28, or 31-47, wherein the VL comprises an amino acid sequence comprising at least one, two, three, four, or five but no more than 10 modifications, e.g., substitutions (e.g., conservative substitutions) relative to the amino acid sequence of SEQ ID NO: 73.
   49. The antibody of any one of embodiments 1-23, 26-28, or 31-48, wherein the VL comprises an amino acid sequence comprising at least one, two, three, four, or five but no more than 10 different amino acids relative to the amino acid sequence of SEQ ID NO: 73.
   50. The antibody of any one of embodiments 1-22, 24, 26, 27, 29 or 31-46, wherein VL comprises:
(i) the amino acid sequence of SEQ ID NO: 72; or an amino acid sequence at least 91% (e.g., at least 92, 95, 96, 97, 98, or 99%) identical to the amino acid sequence of SEQ ID NO: 72;
(ii) an amino acid sequence comprising at least one, two, three, four, or five but no more than 10 modifications, e.g., substitutions (e.g., conservative substitutions) relative to the amino acid sequence of SEQ ID NO: 72;
(iii) an amino acid sequence comprising at least one, two, three, four, or five but no more than 10 different amino acids relative to the amino acid sequence of SEQ ID NO: 72.
   51. The antibody of any one of embodiments 1-22 or 24-27, or 30-46, wherein VL comprises:
(i) the amino acid sequence of SEQ ID NO: 74; or an amino acid sequence at least 91% (e.g., at least 92, 95, 96, 97, 98, or 99%) identical to the amino acid sequence of SEQ ID NO: 74;
(ii) an amino acid sequence comprising at least one, two, three, four, or five but no more than 10 modifications, e.g., substitutions (e.g., conservative substitutions) relative to the amino acid sequence of SEQ ID NO: 74;
(iii) an amino acid sequence comprising at least one, two, three, four, or five but no more than 10 different amino acids relative to the amino acid sequence of SEQ ID NO: 74.
   52. The antibody of any one of embodiments 1-51, wherein:
(a) the VH comprises the amino acid sequence of any one of SEQ ID NOs: 67-71, or an amino acid sequence at least 86% (e.g., at least 90, 92, 95, 96, 97, 98, or 99%) identical to the amino acid sequence of any one of SEQ ID NOs: 67-71; an amino acid sequence comprising at least one, two, three, four, or five but no more than 10 modifications, e.g., substitutions (e.g., conservative substitutions) relative to the amino acid sequence of any one of SEQ ID NOs: 67-71; or an amino acid sequence comprising at least one, two, three, four, or five but no more than 16 different amino acids relative to the amino acid sequence of any one of SEQ ID NOs: 67-71; and
(b) the VL comprises the amino acid sequence of any one of SEQ ID NOs: 72-76; or an amino acid sequence at least 91% (e.g., at least 92, 95, 96, 97, 98, or 99%) identical to the amino acid sequence of any one of SEQ ID NOs: 72-76; an amino acid sequence comprising at least one, two, three, four, or five but no more than 10 modifications, e.g., substitutions (e.g., conservative substitutions) relative to the amino acid sequence of any one of SEQ ID NOs: 72-76; or an amino acid sequence comprising at least one, two, three, four, or five but no more than 10 different amino acids relative to the amino acid sequence of any one of SEQ ID NOs: 72-76.
   53. The antibody of any one of embodiments 1-52, wherein:
(a) the VH comprises the amino acid sequence of any one of SEQ ID NOs: 67 or 69-71, or an amino acid sequence at least 86% (e.g., at least 90, 92, 95, 96, 97, 98, or 99%) identical to the amino acid sequence of any one of SEQ ID NOs: 67 or 69-71; an amino acid sequence comprising at least one, two, three, four, or five but no more than 10 modifications, e.g., substitutions (e.g., conservative substitutions) relative to the amino acid sequence of any one of SEQ ID NOs: 67 or 69-71; or an amino acid sequence comprising at least one, two, three, four, or five but no more than 16 different amino acids relative to the amino acid sequence of any one of SEQ ID NOs: 67 or 69-71; and
(b) the VL comprises the amino acid sequence of any one of SEQ ID NOs: 72-74; or an amino acid sequence at least 91% (e.g., at least 92, 95, 96, 97, 98, or 99%) identical to the amino acid sequence of any one of SEQ ID NOs: 72-74; an amino acid sequence comprising at least one, two, three, four, or five but no more than 10 modifications, e.g., substitutions (e.g., conservative substitutions) relative to the amino acid sequence of any one of SEQ ID NOs: 72-74; or an amino acid sequence comprising at least one, two, three, four, or five but no more than 10 different amino acids relative to the amino acid sequence of any one of SEQ ID NOs: 72-74.
   54. The antibody of any one of embodiments 1-53, wherein:
(a) the VH comprises the amino acid sequence of any one of SEQ ID NOs: 67-71, or an amino acid sequence at least 95%, 96%, 97%, 98%, or 99% identical to the amino acid sequence of any one of SEQ ID NOs: 67-71; and
(b) the VL comprises the amino acid sequence of any one of SEQ ID NOs: 72-76; or an amino acid sequence at least 95%, 96%, 97%, 98%, or 99% identical to the amino acid sequence of any one of SEQ ID NOs: 72-76.
   55. The antibody of any one of embodiments 1-54, wherein:
(a) the VH comprises the amino acid sequence of any one of SEQ ID NOs: 67 or 69-71, or an amino acid sequence at least 95%, 96%, 97%, 98%, or 99% identical to the amino acid sequence of any one of SEQ ID NOs: 67 or 69-71; and
(b) the VL comprises the amino acid sequence of any one of SEQ ID NOs: 72-74; or an amino acid sequence at least 95%, 96%, 97%, 98%, or 99% identical to the amino acid sequence of any one of SEQ ID NOs: 72-74.
   56. The antibody of any one of embodiments 1-58, wherein:
(i) the VH comprises the amino acid sequence of SEQ ID NO: 67, or an amino acid sequence at least 86%, 87%, 88%, 90%, 95%, 96%, 97%, 98%, or 99% identical to the amino acid sequence of SEQ ID NO: 67; and the VL comprises the amino acid sequence of SEQ ID NO: 72, or an amino acid sequence at least 91%, 93%, 95%, 96%, 97%, 98%, or 99% identical to the amino acid sequence of SEQ ID NO: 72;
(ii) the VH comprises the amino acid sequence of SEQ ID NO: 67, or an amino acid sequence at least 86%, 87%, 88%, 90%, 95%, 96%, 97%, 98%, or 99% identical to the amino acid sequence of SEQ ID NO: 67; and the VL comprises the amino acid sequence of SEQ ID NO: 73, or an amino acid sequence at least 91%, 93%, 95%, 96%, 97%, 98%, or 99% identical to the amino acid sequence of SEQ ID NO: 73;
(iii) the VH comprises the amino acid sequence of SEQ ID NO: 67, or an amino acid sequence at least 86%, 87%, 88%, 90%, 95%, 96%, 97%, 98%, or 99% identical to the amino acid sequence of SEQ ID NO: 67; and the VL comprises the amino acid sequence of SEQ ID NO: 74, or an amino acid sequence at least 91%, 93%, 95%, 96%, 97%, 98%, or 99% identical to the amino acid sequence of SEQ ID NO: 74;
(iv) the VH comprises the amino acid sequence of SEQ ID NO: 67, or an amino acid sequence at least 86%, 87%, 88%, 90%, 95%, 96%, 97%, 98%, or 99% identical to the amino acid sequence of SEQ ID NO: 67; and the VL comprises the amino acid sequence of SEQ ID NO: 75, or an amino acid sequence at least 91%, 93%, 95%, 96%, 97%, 98%, or 99% identical to the amino acid sequence of SEQ ID NO: 75;
(v) the VH comprises the amino acid sequence of SEQ ID NO: 67, or an amino acid sequence at least 86%, 87%, 88%, 90%, 95%, 96%, 97%, 98%, or 99% identical to the amino acid sequence of SEQ ID NO: 67; and the VL comprises the amino acid sequence of SEQ ID NO: 76, or an amino acid sequence at least 91%, 93%, 95%, 96%, 97%, 98%, or 99% identical to the amino acid sequence of SEQ ID NO: 76;
(vi) the VH comprises the amino acid sequence of SEQ ID NO: 68, or an amino acid sequence at least 86%, 87%, 88%, 90%, 95%, 96%, 97%, 98%, or 99% identical to the amino acid sequence of SEQ ID NO: 68; and the VL comprises the amino acid sequence of SEQ ID NO: 72, or an amino acid sequence at least 91%, 93%, 95%, 96%, 97%, 98%, or 99% identical to the amino acid sequence of SEQ ID NO: 72;
(vii) the VH comprises the amino acid sequence of SEQ ID NO: 68, or an amino acid sequence at least 86%, 87%, 88%, 90%, 95%, 96%, 97%, 98%, or 99% identical to the amino acid sequence of SEQ ID NO: 68; and the VL comprises the amino acid sequence of SEQ ID NO: 73, or an amino acid sequence at least 91%, 93%, 95%, 96%, 97%, 98%, or 99% identical to the amino acid sequence of SEQ ID NO: 73;
(viii) the VH comprises the amino acid sequence of SEQ ID NO: 68, or an amino acid sequence at least 86%, 87%, 88%, 90%, 95%, 96%, 97%, 98%, or 99% identical to the amino acid sequence of SEQ ID NO: 68; and the VL comprises the amino acid sequence of SEQ ID NO: 74, or an amino acid sequence at least 91%, 93%, 95%, 96%, 97%, 98%, or 99% identical to the amino acid sequence of SEQ ID NO: 74;
(ix) the VH comprises the amino acid sequence of SEQ ID NO: 68, or an amino acid sequence at least 86%, 87%, 88%, 90%, 95%, 96%, 97%, 98%, or 99% identical to the amino acid sequence of SEQ ID NO: 68; and the VL comprises the amino acid sequence of SEQ ID NO: 75, or an amino acid sequence at least 91%, 93%, 95%, 96%, 97%, 98%, or 99% identical to the amino acid sequence of SEQ ID NO: 75;
(x) the VH comprises the amino acid sequence of SEQ ID NO: 68, or an amino acid sequence at least 86%, 87%, 88%, 90%, 95%, 96%, 97%, 98%, or 99% identical to the amino acid sequence of SEQ ID NO: 68; and the VL comprises the amino acid sequence of SEQ ID NO: 76, or an amino acid sequence at least 91%, 93%, 95%, 96%, 97%, 98%, or 99% identical to the amino acid sequence of SEQ ID NO: 76;
(xi) the VH comprises the amino acid sequence of SEQ ID NO: 69, or an amino acid sequence at least 86%, 87%, 88%, 90%, 95%, 96%, 97%, 98%, or 99% identical to the amino acid sequence of SEQ ID NO: 69; and the VL comprises the amino acid sequence of SEQ ID NO: 72, or an amino acid sequence at least 91%, 93%, 95%, 96%, 97%, 98%, or 99% identical to the amino acid sequence of SEQ ID NO: 72;
(xii) the VH comprises the amino acid sequence of SEQ ID NO: 69, or an amino acid sequence at least 86%, 87%, 88%, 90%, 95%, 96%, 97%, 98%, or 99% identical to the amino acid sequence of SEQ ID NO: 69; and the VL comprises the amino acid sequence of SEQ ID NO: 73, or an amino acid sequence at least 91%, 93%, 95%, 96%, 97%, 98%, or 99% identical to the amino acid sequence of SEQ ID NO: 73;
(xiii) the VH comprises the amino acid sequence of SEQ ID NO: 69, or an amino acid sequence at least 86%, 87%, 88%, 90%, 95%, 96%, 97%, 98%, or 99% identical to the amino acid sequence of SEQ ID NO: 69; and the VL comprises the amino acid sequence of SEQ ID NO: 74, or an amino acid sequence at least 91%, 93%, 95%, 96%, 97%, 98%, or 99% identical to the amino acid sequence of SEQ ID NO: 74;
(xiv) the VH comprises the amino acid sequence of SEQ ID NO: 69, or an amino acid sequence at least 86%, 87%, 88%, 90%, 95%, 96%, 97%, 98%, or 99% identical to the amino acid sequence of SEQ ID NO: 69; and the VL comprises the amino acid sequence of SEQ ID NO: 75, or an amino acid sequence at least 91%, 93%, 95%, 96%, 97%, 98%, or 99% identical to the amino acid sequence of SEQ ID NO: 75;
(xv) the VH comprises the amino acid sequence of SEQ ID NO: 69, or an amino acid sequence at least 86%, 87%, 88%, 90%, 95%, 96%, 97%, 98%, or 99% identical to the amino acid sequence of SEQ ID NO: 69; and the VL comprises the amino acid sequence of SEQ ID NO: 76, or an amino acid sequence at least 91%, 93%, 95%, 96%, 97%, 98%, or 99% identical to the amino acid sequence of SEQ ID NO: 76;
(xvi) the VH comprises the amino acid sequence of SEQ ID NO: 70, or an amino acid sequence at least 86%, 87%, 88%, 90%, 95%, 96%, 97%, 98%, or 99% identical to the amino acid sequence of SEQ ID NO: 70; and the VL comprises the amino acid sequence of SEQ ID NO: 72, or an amino acid sequence at least 91%, 93%, 95%, 96%, 97%, 98%, or 99% identical to the amino acid sequence of SEQ ID NO: 72;
(xvii) the VH comprises the amino acid sequence of SEQ ID NO: 70, or an amino acid sequence at least 86%, 87%, 88%, 90%, 95%, 96%, 97%, 98%, or 99% identical to the amino acid sequence of SEQ ID NO: 70; and the VL comprises the amino acid sequence of SEQ ID NO: 73, or an amino acid sequence at least 91%, 93%, 95%, 96%, 97%, 98%, or 99% identical to the amino acid sequence of SEQ ID NO: 73;
(xviii) the VH comprises the amino acid sequence of SEQ ID NO: 70, or an amino acid sequence at least 86%, 87%, 88%, 90%, 95%, 96%, 97%, 98%, or 99% identical to the amino acid sequence of SEQ ID NO: 70; and the VL comprises the amino acid sequence of SEQ ID NO: 74, or an amino acid sequence at least 91%, 93%, 95%, 96%, 97%, 98%, or 99% identical to the amino acid sequence of SEQ ID NO: 74;
(xix) the VH comprises the amino acid sequence of SEQ ID NO: 70, or an amino acid sequence at least 86%, 87%, 88%, 90%, 95%, 96%, 97%, 980%, or 99% identical to the amino acid sequence of SEQ ID NO: 70; and the VL comprises the amino acid sequence of SEQ ID NO: 75, or an amino acid sequence at least 91%, 93%, 95%, 96%, 97%, 98%, or 99% identical to the amino acid sequence of SEQ ID NO: 75;
(xx) the VH comprises the amino acid sequence of SEQ ID NO: 70, or an amino acid sequence at least 86%, 87%, 88%, 90%, 95%, 96%, 97%, 98%, or 99% identical to the amino acid sequence of SEQ ID NO: 70; and the VL comprises the amino acid sequence of SEQ ID NO: 76, or an amino acid sequence at least 91%, 93%, 95%, 96%, 97%, 98%, or 99% identical to the amino acid sequence of SEQ ID NO: 76;
(xxi) the VH comprises the amino acid sequence of SEQ ID NO: 71, or an amino acid sequence at least 86%, 87%, 88%, 90%, 95%, 96%, 97%, 98%, or 99% identical to the amino acid sequence of SEQ ID NO: 71; and the VL comprises the amino acid sequence of SEQ ID NO: 72, or an amino acid sequence at least 91%, 93%, 95%, 96%, 97%, 98%, or 99% identical to the amino acid sequence of SEQ ID NO: 72;
(xxii) the VH comprises the amino acid sequence of SEQ ID NO: 71, or an amino acid sequence at least 86%, 87%, 88%, 90%, 95%, 96%, 97%, 98%, or 99% identical to the amino acid sequence of SEQ ID NO: 71; and the VL comprises the amino acid sequence of SEQ ID NO: 73, or an amino acid sequence at least 91%, 93%, 95%, 96%, 97%, 98%, or 99% identical to the amino acid sequence of SEQ ID NO: 73;
(xxiii) the VH comprises the amino acid sequence of SEQ ID NO: 71, or an amino acid sequence at least 86%, 87%, 88%, 90%, 95%, 96%, 97%, 98%, or 99% identical to the amino acid sequence of SEQ ID NO: 71; and the VL comprises the amino acid sequence of SEQ ID NO: 74, or an amino acid sequence at least 91%, 93%, 95%, 96%, 97%, 98%, or 99% identical to the amino acid sequence of SEQ ID NO: 74;
(xxiv) the VH comprises the amino acid sequence of SEQ ID NO: 71, or an amino acid sequence at least 86%, 87%, 88%, 90%, 95%, 96%, 97%, 98%, or 99% identical to the amino acid sequence of SEQ ID NO: 71; and the VL comprises the amino acid sequence of SEQ ID NO: 75, or an amino acid sequence at least 91%, 93%, 95%, 96%, 97%, 98%, or 99% identical to the amino acid sequence of SEQ ID NO: 75; or
(xv) the VH comprises the amino acid sequence of SEQ ID NO: 71, or an amino acid sequence at least 86%, 87%, 88%, 90%, 95%, 96%, 97%, 98%, or 99% identical to the amino acid sequence of SEQ ID NO: 71; and the VL comprises the amino acid sequence of SEQ ID NO: 76, or an amino acid sequence at least 91%, 93%, 95%, 96%, 97%, 98%, or 99% identical to the amino acid sequence of SEQ ID NO: 76.
   57. The antibody of any one of embodiments 1-11, 15-17, 21-23, 26-28, 31-38, 44-49, or 52-56, wherein:
(i) the VH comprises the amino acid sequence of SEQ ID NO: 69, or an amino acid sequence at least 86%, 87%, 88%, 90%, 95%, 96%, 97%, 98%, or 99% identical to the amino acid sequence of SEQ ID NO: 69; an amino acid sequence comprising at least one, two, three, four, or five but no more than 10 modifications, e.g., substitutions (e.g., conservative substitutions) relative to the amino acid sequence of SEQ ID NO: 69; or an amino acid sequence comprising at least one, two, three, four, or five but no more than 16 different amino acids relative to the amino acid sequence of any one of SEQ ID NO: 69; and
(ii) the VL comprises the amino acid sequence of SEQ ID NO: 73, or an amino acid sequence at least 91%, 93%, 95%, 96%, 97%, 98%, or 99% identical to the amino acid sequence of SEQ ID NO: 73; an amino acid sequence comprising at least one, two, three, four, or five but no more than 10 modifications, e.g., substitutions (e.g., conservative substitutions) relative to the amino acid sequence of SEQ ID NO: 73; or an amino acid sequence comprising at least one, two, three, four, or five but no more than 16 different amino acids relative to the amino acid sequence of any one of SEQ ID NO: 73.
   58. The antibody of any one of embodiments 1-11, 15-17, 21-23, 26-28, 31-38, 44-49, or 52-57, wherein the VH comprises the amino acid sequence of SEQ ID NO: 69 and the VL comprises the amino acid sequence of SEQ ID NO: 73.
   59. The antibody of any one of embodiments 1-10, 12, 15, 16, 18, 21, 22, 24, 26, 27, 29, 31-37, 41, 44-46, 50, or 52-56, wherein:
(i) the VH comprises the amino acid sequence of SEQ ID NO: 67, or an amino acid sequence at least 86%, 87%, 88%, 90%, 95%, 96%, 97%, 98%, or 99% identical to the amino acid sequence of SEQ ID NO: 67; an amino acid sequence comprising at least one, two, three, four, or five but no more than 10 modifications, e.g., substitutions (e.g., conservative substitutions) relative to the amino acid sequence of SEQ ID NO: 67; or an amino acid sequence comprising at least one, two, three, four, or five but no more than 16 different amino acids relative to the amino acid sequence of any one of SEQ ID NO: 67; and
(ii) the VL comprises the amino acid sequence of SEQ ID NO: 72, or an amino acid sequence at least 91%, 93%, 95%, 96%, 97%, 98%, or 99% identical to the amino acid sequence of SEQ ID NO: 72; an amino acid sequence comprising at least one, two, three, four, or five but no more than 10 modifications, e.g., substitutions (e.g., conservative substitutions) relative to the amino acid sequence of SEQ ID NO: 72; or an amino acid sequence comprising at least one, two, three, four, or five but no more than 16 different amino acids relative to the amino acid sequence of any one of SEQ ID NO: 72.
   60. The antibody of any one of embodiments 1-10, 13, 15, 16, 19, 21, 22, 24, 26, 27, 29, 31-37, 42, 44-46, 50, or 52-56, wherein:
(i) the VH comprises the amino acid sequence of SEQ ID NO: 70, or an amino acid sequence at least 86%, 87%, 88%, 90%, 95%, 96%, 97%, 98%, or 99% identical to the amino acid sequence of SEQ ID NO: 70; an amino acid sequence comprising at least one, two, three, four, or five but no more than 10 modifications, e.g., substitutions (e.g., conservative substitutions) relative to the amino acid sequence of SEQ ID NO: 70; or an amino acid sequence comprising at least one, two, three, four, or five but no more than 16 different amino acids relative to the amino acid sequence of any one of SEQ ID NO: 70; and
(ii) the VL comprises the amino acid sequence of SEQ ID NO: 72, or an amino acid sequence at least 91%, 93%, 95%, 96%, 97%, 98%, or 99% identical to the amino acid sequence of SEQ ID NO: 72; an amino acid sequence comprising at least one, two, three, four, or five but no more than 10 modifications, e.g., substitutions (e.g., conservative substitutions) relative to the amino acid sequence of SEQ ID NO: 72; or an amino acid sequence comprising at least one, two, three, four, or five but no more than 16 different amino acids relative to the amino acid sequence of any one of SEQ ID NO: 72.
   61. The antibody of any one of embodiments 1-10, 14-16, 20-23, 31-37, 43-49, or 52-56, wherein:
(i) the VH comprises the amino acid sequence of SEQ ID NO: 71, or an amino acid sequence at least 86%, 87%, 88%, 90%, 95%, 96%, 97%, 98%, or 99% identical to the amino acid sequence of SEQ ID NO: 71; an amino acid sequence comprising at least one, two, three, four, or five but no more than 10 modifications, e.g., substitutions (e.g., conservative substitutions) relative to the amino acid sequence of SEQ ID NO: 71; or an amino acid sequence comprising at least one, two, three, four, or five but no more than 16 different amino acids relative to the amino acid sequence of any one of SEQ ID NO: 71; and
(ii) the VL comprises the amino acid sequence of SEQ ID NO: 73, or an amino acid sequence at least 91%, 93%, 95%, 96%, 97%, 98%, or 99% identical to the amino acid sequence of SEQ ID NO: 73; an amino acid sequence comprising at least one, two, three, four, or five but no more than 10 modifications, e.g., substitutions (e.g., conservative substitutions) relative to the amino acid sequence of SEQ ID NO: 73; or an amino acid sequence comprising at least one, two, three, four, or five but no more than 16 different amino acids relative to the amino acid sequence of any one of SEQ ID NO: 73.
   62. The antibody of any one of embodiments 1-10, 14-16, 20-22, 25-27, 30-37, 43-46, or 51-56, wherein:
(i) the VH comprises the amino acid sequence of SEQ ID NO: 71, or an amino acid sequence at least 86%, 87%, 88%, 90%, 95%, 96%, 97%, 98%, or 99% identical to the amino acid sequence of SEQ ID NO: 71; an amino acid sequence comprising at least one, two, three, four, or five but no more than 10 modifications, e.g., substitutions (e.g., conservative substitutions) relative to the amino acid sequence of SEQ ID NO: 71; or an amino acid sequence comprising at least one, two, three, four, or five but no more than 16 different amino acids relative to the amino acid sequence of any one of SEQ ID NO: 71; and
(ii) the VL comprises the amino acid sequence of SEQ ID NO: 74, or an amino acid sequence at least 91%, 93%, 95%, 96%, 97%, 98%, or 99% identical to the amino acid sequence of SEQ ID NO: 74; an amino acid sequence comprising at least one, two, three, four, or five but no more than 10 modifications, e.g., substitutions (e.g., conservative substitutions) relative to the amino acid sequence of SEQ ID NO: 74; or an amino acid sequence comprising at least one, two, three, four, or five but no more than 16 different amino acids relative to the amino acid sequence of any one of SEQ ID NO: 74.
   63. The antibody of any one of embodiments 1-62, wherein:
(i) the nucleotide sequence encoding the VH comprises the nucleotide sequence of any one of SEQ ID NOs: 156-160, or a nucleotide sequence at least 80% (e.g., at least 85%, 87%, 90%, 91%, 95%, 96%, 97%, 98%, or 99%) identical thereto; and/or
(ii) the nucleotide sequence encoding the VL comprises the nucleotide sequence of any one of SEQ ID NOs: 161-165; or a nucleotide sequence at least 80% (e.g., at least 85%, 87%, 90%, 91%, 95%, 96%, 97%, 98%, or 99%) identical thereto.
   64. The antibody of any one of embodiments 1-63, wherein:
(i) the nucleotide sequence encoding the VH comprises the nucleotide sequence of any one of SEQ ID NOs: 156 or 158-160, or a nucleotide sequence at least 80% (e.g., at least 85%, 87%, 90%, 91%, 95%, 96%, 97%, 98%, or 99%) identical thereto; and/or
(ii) the nucleotide sequence encoding the VL comprises the nucleotide sequence of any one of SEQ ID NOs: 161-163; or a nucleotide sequence at least 80% (e.g., at least 85%, 87%, 90%, 91%, 95%, 96%, 97%, 98%, or 99%) identical thereto.
   65. The antibody of any one of embodiments 1-11, 15-17, 21-23, 26-28, 31-38, 44-49, 52-58, 63, or 64, wherein:
(i) the nucleotide sequence encoding the VH comprises the nucleotide sequence of SEQ ID NO: 158, or a nucleotide sequence at least 80% (e.g., at least 85%, 87%, 90%, 91%, 95%, 96%, 97%, 98%, or 99%) identical thereto; and/or
(ii) the nucleotide sequence encoding the VL comprises the nucleotide sequence of any one of SEQ ID NO: 162; or a nucleotide sequence at least 80% (e.g., at least 85%, 87%, 90%, 91%, 95%, 96%, 97%, 98%, or 99%) identical thereto.
   66. The antibody of any one of embodiments 1-10, 12, 15, 16, 18, 21, 22, 24, 26, 27, 29, 31-37, 41, 44-46, 50, 52-56, 59, 63, or 64, wherein:
(i) the nucleotide sequence encoding the VH comprises the nucleotide sequence of SEQ ID NO: 156, or a nucleotide sequence at least 80% (e.g., at least 85%, 87%, 90%, 91%, 95%, 96%, 97%, 98%, or 99%) identical thereto; and/or
(ii) the nucleotide sequence encoding the VL comprises the nucleotide sequence of any one of SEQ ID NO: 161; or a nucleotide sequence at least 80% (e.g., at least 85%, 87%, 90%, 91%, 95%, 96%, 97%, 98%, or 99%) identical thereto.
   67. The antibody of any one of embodiments 1-10, 13, 15, 16, 19, 21, 22, 24, 26, 27, 29, 31-37, 42, 44-46, 50, 52-56, 60, 63, or 64, wherein:
(i) the nucleotide sequence encoding the VH comprises the nucleotide sequence of SEQ ID NO: 159, or a nucleotide sequence at least 80% (e.g., at least 85%, 87%, 90%, 91%, 95%, 96%, 97%, 98%, or 99%) identical thereto; and/or
(ii) the nucleotide sequence encoding the VL comprises the nucleotide sequence of any one of SEQ ID NO: 161; or a nucleotide sequence at least 80% (e.g., at least 85%, 87%, 90%, 91%, 95%, 96%, 97%, 98%, or 99%) identical thereto.
   68. The antibody of any one of embodiments 1-10, 14-16, 20-23, 25-27, 30-37,43-49, 51-56, 61-64, wherein:
(i) the nucleotide sequence encoding the VH comprises the nucleotide sequence of SEQ ID NO: 160, or a nucleotide sequence at least 80% (e.g., at least 85%, 87%, 90%, 91%, 95%, 96%, 97%, 98%, or 99%) identical thereto; and/or
(ii) the nucleotide sequence encoding the VL comprises the nucleotide sequence of any one of SEQ ID NO: 162 or 163; or a nucleotide sequence at least 80% (e.g., at least 85%, 87%, 90%, 91%, 95%, 96%, 97%, 98%, or 99%) identical thereto.
   69. The antibody of any one of the preceding embodiments, which does not comprise the amino acid sequence of SEQ ID NO: 21 and/or the amino acid sequence of SEQ ID NO: 93.
   70. An antibody that binds to human tau, which comprises:
(a) a heavy chain variable region (VH) comprising a heavy chain complementarity determining region 1 (HC CDR1) comprising the amino acid sequence of SEQ ID NO: 64, a heavy chain complementarity determining region 2 (HC CDR2) comprising the amino acid sequence of SEQ ID NO: 1145, and a heavy chain complementarity determining region 3 (HC CDR3) comprising the amino acid sequence of SEQ ID NO: 1167; and a light chain variable region (VL) comprising a light chain complementarity determining region 1 (LC CDR1) comprising the amino acid sequence of SEQ ID NO: 1146, a light chain complementarity determining region 2 (LC CDR2) comprising the amino acid sequence of SEQ ID NO: 529, and a light chain complementarity determining region 3 (LC CDR3) comprising the amino acid sequence of SEQ ID NO: 571;
(b) a VH comprising a HC CDR1 comprising the amino acid sequence of SEQ ID NO: 1144, a HC CDR2 comprising the amino acid sequence of SEQ ID NO: 1145, and a HC CDR3 comprising the amino acid sequence of SEQ ID NO: 410; and a VL comprising a LC CDR1 comprising the amino acid sequence of SEQ ID NO: 1146, a LC CDR2 comprising the amino acid sequence of SEQ ID NO: 529, and a LC CDR3 comprising the amino acid sequence of SEQ ID NO: 571;
(c) a VH comprising a HC CDR1 comprising the amino acid sequence of SEQ ID NO: 1165, a HC CDR2 comprising the amino acid sequence of SEQ ID NO: 1166, and a HC CDR3 comprising the amino acid sequence of SEQ ID NO: 1167; and a VL comprising a LC CDR1 comprising the amino acid sequence of SEQ ID NO: 473, a LC CDR2 comprising the amino acid sequence of RVS, and a LC CDR3 comprising the amino acid sequence of SEQ ID NO: 571; or
(d) a VH comprising a HC CDR1 comprising the amino acid sequence of SEQ ID NO: 314, a HC CDR2 comprising the amino acid sequence of SEQ ID NO: 341, and a HC CDR3 comprising the amino acid sequence of SEQ ID NO: 410; and a VL comprising a LC CDR1 comprising the amino acid sequence of SEQ ID NO: 1154, a LC CDR2 comprising the amino acid sequence of SEQ ID NO: 529, and a LC CDR3 comprising the amino acid sequence of SEQ ID NO: 571; and
wherein:
(i) the VH comprises 1, 2, 3, 4, 5, 6, 7, 8, 9, 10, 11, 12, 13, 14, 15, 16, 17, 18, 19, 20, 21, or all of: V at position 5; S at position 7; A at position 9; V at position 11; K at position 12; S at position 14; A at position 16; K at position 19; V at position 20; R at position 38; Q at position 39; A at position 40; Q at position 43; R at position 67; V at position 68; I at position 71; R at position 72; D at position 73; T at position 74; T at position 76; T at position 84; and/or L at position 113, numbered according to SEQ ID NO: 21; and
(ii) the VL comprises 1, 2, 3, 4, 5, 6, 7, 8, 9, 10, 11, 12, or all of: I at position 2; S at position 7; S at position 12; T at position 14; P at position 15; Q at position 17; P at position 18; Q at position 50; S at position 68; V at position 88; Y at position 92; Q at position 105; and/or V at position 109, numbered according to SEQ ID NO: 93.
   71. An antibody that binds to human tau, which comprises:
(a) a heavy chain variable region (VH) comprising a heavy chain complementarity determining region 1 (HC CDR1) comprising the amino acid sequence of SEQ ID NO: 64, a heavy chain complementarity determining region 2 (HC CDR2) comprising the amino acid sequence of SEQ ID NO: 1145, and a heavy chain complementarity determining region 3 (HC CDR3) comprising the amino acid sequence of SEQ ID NO: 1167; and a light chain variable region (VL) comprising a light chain complementarity determining region 1 (LC CDR1) comprising the amino acid sequence of SEQ ID NO: 1146, a light chain complementarity determining region 2 (LC CDR2) comprising the amino acid sequence of SEQ ID NO: 529, and a light chain complementarity determining region 3 (LC CDR3) comprising the amino acid sequence of SEQ ID NO: 571;
(b) a VH comprising a HC CDR1 comprising the amino acid sequence of SEQ ID NO: 1144, a HC CDR2 comprising the amino acid sequence of SEQ ID NO: 1145, and a HC CDR3 comprising the amino acid sequence of SEQ ID NO: 410; and a VL comprising a LC CDR1 comprising the amino acid sequence of SEQ ID NO: 1146, a LC CDR2 comprising the amino acid sequence of SEQ ID NO: 529, and a LC CDR3 comprising the amino acid sequence of SEQ ID NO: 571;
(c) a VH comprising a HC CDR1 comprising the amino acid sequence of SEQ ID NO: 1165, a HC CDR2 comprising the amino acid sequence of SEQ ID NO: 1166, and a HC CDR3 comprising the amino acid sequence of SEQ ID NO: 1167; and a VL comprising a LC CDR1 comprising the amino acid sequence of SEQ ID NO: 473, a LC CDR2 comprising the amino acid sequence of RVS, and a LC CDR3 comprising the amino acid sequence of SEQ ID NO: 571; or
(d) a VH comprising a HC CDR1 comprising the amino acid sequence of SEQ ID NO: 314, a HC CDR2 comprising the amino acid sequence of SEQ ID NO: 341, and a HC CDR3 comprising the amino acid sequence of SEQ ID NO: 410; and a VL comprising a LC CDR1 comprising the amino acid sequence of SEQ ID NO: 1154, a LC CDR2 comprising the amino acid sequence of SEQ ID NO: 529, and a LC CDR3 comprising the amino acid sequence of SEQ ID NO: 571; and
wherein:
(i) the VH comprises the amino acid sequence of SEQ ID NO: 69, or an amino acid sequence at least 86% (e.g., at least 90, 92, 95, 96, 97, 98, or 99%) identical to the amino acid sequence of SEQ ID NO: 69; and/or
(ii) the VL comprises the amino acid sequence of SEQ ID NO: 73, or an amino acid sequence at least 91% (e.g., at least 92, 95, 96, 97, 98, or 99/6) identical to the amino acid sequence of SEQ ID NO: 73.
   72. An antibody that binds to human tau, which comprises a heavy chain variable region (VH) comprising the amino acid sequence of SEQ ID NO: 69, and a light chain variable region (VL) comprising the amino acid sequence of SEQ ID NO: 73.
   73. An antibody that binds to human tau, which comprises:
(a) a heavy chain variable region (VH) comprising a heavy chain complementarity determining region 1 (HC CDR1) comprising the amino acid sequence of SEQ ID NO: 64, a heavy chain complementarity determining region 2 (HC CDR2) comprising the amino acid sequence of SEQ ID NO: 1145, and a heavy chain complementarity determining region 3 (HC CDR3) comprising the amino acid sequence of SEQ ID NO: 1167; and a light chain variable region (VL) comprising a light chain complementarity determining region 1 (LC CDR1) comprising the amino acid sequence of SEQ ID NO: 1146, a light chain complementarity determining region 2 (LC CDR2) comprising the amino acid sequence of SEQ ID NO: 529, and a light chain complementarity determining region 3 (LC CDR3) comprising the amino acid sequence of SEQ ID NO: 571;
(b) a VH comprising a HC CDR1 comprising the amino acid sequence of SEQ ID NO: 1144, a HC CDR2 comprising the amino acid sequence of SEQ ID NO: 1145, and a HC CDR3 comprising the amino acid sequence of SEQ ID NO: 410; and a VL comprising a LC CDR1 comprising the amino acid sequence of SEQ ID NO: 1146, a LC CDR2 comprising the amino acid sequence of SEQ ID NO: 529, and a LC CDR3 comprising the amino acid sequence of SEQ ID NO: 571;
(c) a VH comprising a HC CDR1 comprising the amino acid sequence of SEQ ID NO: 1165, a HC CDR2 comprising the amino acid sequence of SEQ ID NO: 1166, and a HC CDR3 comprising the amino acid sequence of SEQ ID NO: 1167; and a VL comprising a LC CDR1 comprising the amino acid sequence of SEQ ID NO: 473, a LC CDR2 comprising the amino acid sequence of RVS, and a LC CDR3 comprising the amino acid sequence of SEQ ID NO: 571; or
(d) a VH comprising a HC CDR1 comprising the amino acid sequence of SEQ ID NO: 314, a HC CDR2 comprising the amino acid sequence of SEQ ID NO: 341, and a HC CDR3 comprising the amino acid sequence of SEQ ID NO: 410; and a VL comprising a LC CDR1 comprising the amino acid sequence of SEQ ID NO: 1154, a LC CDR2 comprising the amino acid sequence of SEQ ID NO: 529, and a LC CDR3 comprising the amino acid sequence of SEQ ID NO: 571; and
wherein:
(i) the VH comprises 1, 2, 3, 4, 5, 6, 7, 8, 9, 10, 11, 12, 13, 14, 15, 16, 17, 18, 19, 20, 21, or all of: V at position 5; E at position 6; S at position 7; A at position 9; V at position 11; K at position 12; A at position 16; K at position 19; V at position 20; M at position 48; R at position 67; V at position 68; I at position 70; A at position 76; S at position 77; A at position 79; Y at position 80; M at position 81; E at position 82; R at position 87; T at position 91; and/or T at position 113, numbered according to SEQ ID NO: 21; and
(ii) the VL comprises 1, 2, 3, 4, 5, 6, 7, 8, 9, 10, or all of: S at position 7; T at position 14; Q at position 17; P at position 18; Q at position 42; R at position 44; R at position 50; S at position 68; V at position 88; Y at position 92; and/or Q at position 105, numbered according to SEQ ID NO: 93.
   74. An antibody that binds to human tau, which comprises:
(a) a heavy chain variable region (VH) comprising a heavy chain complementarity determining region 1 (HC CDR1) comprising the amino acid sequence of SEQ ID NO: 64, a heavy chain complementarity determining region 2 (HC CDR2) comprising the amino acid sequence of SEQ ID NO: 1145, and a heavy chain complementarity determining region 3 (HC CDR3) comprising the amino acid sequence of SEQ ID NO: 1167; and a light chain variable region (VL) comprising a light chain complementarity determining region 1 (LC CDR1) comprising the amino acid sequence of SEQ ID NO: 1146, a light chain complementarity determining region 2 (LC CDR2) comprising the amino acid sequence of SEQ ID NO: 529, and a light chain complementarity determining region 3 (LC CDR3) comprising the amino acid sequence of SEQ ID NO: 571;
(b) a VH comprising a HC CDR1 comprising the amino acid sequence of SEQ ID NO: 1144, a HC CDR2 comprising the amino acid sequence of SEQ ID NO: 1145, and a HC CDR3 comprising the amino acid sequence of SEQ ID NO: 410; and a VL comprising a LC CDR1 comprising the amino acid sequence of SEQ ID NO: 1146, a LC CDR2 comprising the amino acid sequence of SEQ ID NO: 529, and a LC CDR3 comprising the amino acid sequence of SEQ ID NO: 571;
(c) a VH comprising a HC CDR1 comprising the amino acid sequence of SEQ ID NO: 1165, a HC CDR2 comprising the amino acid sequence of SEQ ID NO: 1166, and a HC CDR3 comprising the amino acid sequence of SEQ ID NO: 1167; and a VL comprising a LC CDR1 comprising the amino acid sequence of SEQ ID NO: 473, a LC CDR2 comprising the amino acid sequence of RVS, and a LC CDR3 comprising the amino acid sequence of SEQ ID NO: 571; or
(d) a VH comprising a HC CDR1 comprising the amino acid sequence of SEQ ID NO: 314, a HC CDR2 comprising the amino acid sequence of SEQ ID NO: 341, and a HC CDR3 comprising the amino acid sequence of SEQ ID NO: 410; and a VL comprising a LC CDR1 comprising the amino acid sequence of SEQ ID NO: 1154, a LC CDR2 comprising the amino acid sequence of SEQ ID NO: 529, and a LC CDR3 comprising the amino acid sequence of SEQ ID NO: 571; and
wherein:
(i) the VH comprises the amino acid sequence of SEQ ID NO: 67, or an amino acid sequence at least 86% (e.g., at least 90, 92, 95, 96, 97, 98, or 99%) identical to the amino acid sequence of SEQ ID NO: 67; and/or
(ii) the VL comprises the amino acid sequence of SEQ ID NO: 72, or an amino acid sequence at least 91% (e.g., at least 92, 95, 96, 97, 98, or 99/6) identical to the amino acid sequence of SEQ ID NO: 72.
   75. An antibody that binds to human tau, which comprises a heavy chain variable region (VH) comprising the amino acid sequence of SEQ ID NO: 67, and a light chain variable region (VL) comprising the amino acid sequence of SEQ ID NO: 72.
   76. An antibody that binds to human tau, which comprises:
(a) a heavy chain variable region (VH) comprising a heavy chain complementarity determining region 1 (HC CDR1) comprising the amino acid sequence of SEQ ID NO: 64, a heavy chain complementarity determining region 2 (HC CDR2) comprising the amino acid sequence of SEQ ID NO: 1145, and a heavy chain complementarity determining region 3 (HC CDR3) comprising the amino acid sequence of SEQ ID NO: 1167; and a light chain variable region (VL) comprising a light chain complementarity determining region 1 (LC CDR1) comprising the amino acid sequence of SEQ ID NO: 1146, a light chain complementarity determining region 2 (LC CDR2) comprising the amino acid sequence of SEQ ID NO: 529, and a light chain complementarity determining region 3 (LC CDR3) comprising the amino acid sequence of SEQ ID NO: 571;
(b) a VH comprising a HC CDR1 comprising the amino acid sequence of SEQ ID NO: 1144, a HC CDR2 comprising the amino acid sequence of SEQ ID NO: 1145, and a HC CDR3 comprising the amino acid sequence of SEQ ID NO: 410; and a VL comprising a LC CDR1 comprising the amino acid sequence of SEQ ID NO: 1146, a LC CDR2 comprising the amino acid sequence of SEQ ID NO: 529, and a LC CDR3 comprising the amino acid sequence of SEQ ID NO: 571;
(c) a VH comprising a HC CDR1 comprising the amino acid sequence of SEQ ID NO: 1165, a HC CDR2 comprising the amino acid sequence of SEQ ID NO: 1166, and a HC CDR3 comprising the amino acid sequence of SEQ ID NO: 1167; and a VL comprising a LC CDR1 comprising the amino acid sequence of SEQ ID NO: 473, a LC CDR2 comprising the amino acid sequence of RVS, and a LC CDR3 comprising the amino acid sequence of SEQ ID NO: 571; or
(d) a VH comprising a HC CDR1 the amino acid sequence of SEQ ID NO: 314, a HC CDR2 comprising the amino acid sequence of SEQ ID NO: 341, and a HC CDR3 comprising the amino acid sequence of SEQ ID NO: 410; and a VL comprising a LC CDR1 comprising the amino acid sequence of SEQ ID NO: 1154, a LC CDR2 comprising the amino acid sequence of SEQ ID NO: 529, and a LC CDR3 comprising the amino acid sequence of SEQ ID NO: 571; and
wherein:
(i) the VH comprises 1, 2, 3, 4, 5, 6, 7, 8, 9, 10, 11, 12, 13, 14, 15, 16, 17, 18, 19, or all of: E at position 1; V at position 5; S at position 7; A at position 9; V at position 11; K at position 12; K at position 19; V at position 20; R at position 67; V at position 68; M at position 70; I at position 76; A at position 79; Y at position 80; M at position 81; E at position 82; R at position 85; D at position 89, L at position 113; and/or S at position 115, numbered according to SEQ ID NO: 21; and
(ii) the VL comprises 1, 2, 3, 4, 5, 6, 7, 8, 9, 10, or all of: S at position 7; T at position 14; Q at position 17; P at position 18; Q at position 42; R at position 44; R at position 50; S at position 68; V at position 88; Y at position 92; and/or Q at position 105, numbered according to SEQ ID NO: 93.
   77. An antibody that binds to human tau, which comprises:
(a) a heavy chain variable region (VH) comprising a heavy chain complementarity determining region 1 (HC CDR1) comprising the amino acid sequence of SEQ ID NO: 64, a heavy chain complementarity determining region 2 (HC CDR2) comprising the amino acid sequence of SEQ ID NO: 1145, and a heavy chain complementarity determining region 3 (HC CDR3) comprising the amino acid sequence of SEQ ID NO: 1167; and a light chain variable region (VL) comprising a light chain complementarity determining region 1 (LC CDR1) comprising the amino acid sequence of SEQ ID NO: 1146, a light chain complementarity determining region 2 (LC CDR2) comprising the amino acid sequence of SEQ ID NO: 529, and a light chain complementarity determining region 3 (LC CDR3) comprising the amino acid sequence of SEQ ID NO: 571;
(b) a VH comprising a HC CDR1 comprising the amino acid sequence of SEQ ID NO: 1144, a HC CDR2 comprising the amino acid sequence of SEQ ID NO: 1145, and a HC CDR3 comprising the amino acid sequence of SEQ ID NO: 410; and a VL comprising a LC CDR1 comprising the amino acid sequence of SEQ ID NO: 1146, a LC CDR2 comprising the amino acid sequence of SEQ ID NO: 529, and a LC CDR3 comprising the amino acid sequence of SEQ ID NO: 571;
(c) a VH comprising a HC CDR1 comprising the amino acid sequence of SEQ ID NO: 1165, a HC CDR2 comprising the amino acid sequence of SEQ ID NO: 1166, and a HC CDR3 comprising the amino acid sequence of SEQ ID NO: 1167; and a VL comprising a LC CDR1 comprising the amino acid sequence of SEQ ID NO: 473, a LC CDR2 comprising the amino acid sequence of RVS, and a LC CDR3 comprising the amino acid sequence of SEQ ID NO: 571; or
(d) a VH comprising a HC CDR1 comprising the amino acid sequence of SEQ ID NO: 314, a HC CDR2 comprising the amino acid sequence of SEQ ID NO: 341, and a HC CDR3 comprising the amino acid sequence of SEQ ID NO: 410; and a VL comprising a LC CDR1 comprising the amino acid sequence of SEQ ID NO: 1154, a LC CDR2 comprising the amino acid sequence of SEQ ID NO: 529, and a LC CDR3 comprising the amino acid sequence of SEQ ID NO: 571; and
wherein:
(i) the VH comprises the amino acid sequence of SEQ ID NO: 70, or an amino acid sequence at least 86% (e.g., at least 90, 92, 95, 96, 97, 98, or 99%) identical to the amino acid sequence of SEQ ID NO: 70; and/or
(ii) the VL comprises the amino acid sequence of SEQ ID NO: 72, or an amino acid sequence at least 91% (e.g., at least 92, 95, 96, 97, 98, or 99/6) identical to the amino acid sequence of SEQ ID NO: 72.
   78. An antibody that binds to human tau, which comprises a heavy chain variable region (VH) comprising the amino acid sequence of SEQ ID NO: 70, and a light chain variable region (VL) comprising the amino acid sequence of SEQ ID NO: 72.
   79. An antibody that binds to human tau, which comprises:
(a) a heavy chain variable region (VH) comprising a heavy chain complementarity determining region 1 (HC CDR1) comprising the amino acid sequence of SEQ ID NO: 64, a heavy chain complementarity determining region 2 (HC CDR2) comprising the amino acid sequence of SEQ ID NO: 1145, and a heavy chain complementarity determining region 3 (HC CDR3) comprising the amino acid sequence of SEQ ID NO: 1167; and a light chain variable region (VL) comprising a light chain complementarity determining region 1 (LC CDR1) comprising the amino acid sequence of SEQ ID NO: 1146, a light chain complementarity determining region 2 (LC CDR2) comprising the amino acid sequence of SEQ ID NO: 529, and a light chain complementarity determining region 3 (LC CDR3) comprising the amino acid sequence of SEQ ID NO: 571;
(b) a VH comprising a HC CDR1 comprising the amino acid sequence of SEQ ID NO: 1144, a HC CDR2 comprising the amino acid sequence of SEQ ID NO: 1145, and a HC CDR3 comprising the amino acid sequence of SEQ ID NO: 410; and a VL comprising a LC CDR1 comprising the amino acid sequence of SEQ ID NO: 1146, a LC CDR2 comprising the amino acid sequence of SEQ ID NO: 529, and a LC CDR3 comprising the amino acid sequence of SEQ ID NO: 571;
(c) a VH comprising a HC CDR1 comprising the amino acid sequence of SEQ ID NO: 1165, a HC CDR2 comprising the amino acid sequence of SEQ ID NO: 1166, and a HC CDR3 comprising the amino acid sequence of SEQ ID NO: 1167; and a VL comprising a LC CDR1 comprising the amino acid sequence of SEQ ID NO: 473, a LC CDR2 comprising the amino acid sequence of RVS, and a LC CDR3 comprising the amino acid sequence of SEQ ID NO: 571; or
(d) a VH comprising a HC CDR1 comprising the amino acid sequence of SEQ ID NO: 314, a HC CDR2 comprising the amino acid sequence of SEQ ID NO: 341, and a HC CDR3 comprising the amino acid sequence of SEQ ID NO: 410; and a VL comprising a LC CDR1 comprising the amino acid sequence of SEQ ID NO: 1154, a LC CDR2 comprising the amino acid sequence of SEQ ID NO: 529, and a LC CDR3 comprising the amino acid sequence of SEQ ID NO: 571; and
wherein:
(i) the VH comprises 1, 2, 3, 4, 5, 6, 7, 8, 9, 10, 11, 12, 13, 14, 15, 16 or all of: V at position 5; S at position 7; A at position 9; V at position 11; K at position 12; A at position 16; K at position 19; V at position 20; Q at position 43; M at position 48; R at position 67; V at position 68; M at position 70; T at position 76; S at position 77; R at position 87; and/or T at position 91, numbered according to SEQ ID NO: 21; and
(ii) the VL comprises 1, 2, 3, 4, 5, 6, 7, 8, 9, 10, 11, 12, or all of: I at position 2; S at position 7; S at position 12; T at position 14; P at position 15; Q at position 17; P at position 18; Q at position 50; S at position 68; V at position 88; Y at position 92; Q at position 105; and/or V at position 109, numbered according to SEQ ID NO: 93.
   80. An antibody that binds to human tau, which comprises:
(a) a heavy chain variable region (VH) comprising a heavy chain complementarity determining region 1 (HC CDR1) comprising the amino acid sequence of SEQ ID NO: 64, a heavy chain complementarity determining region 2 (HC CDR2) comprising the amino acid sequence of SEQ ID NO: 1145, and a heavy chain complementarity determining region 3 (HC CDR3) comprising the amino acid sequence of SEQ ID NO: 1167; and a light chain variable region (VL) comprising a light chain complementarity determining region 1 (LC CDR1) comprising the amino acid sequence of SEQ ID NO: 1146, a light chain complementarity determining region 2 (LC CDR2) comprising the amino acid sequence of SEQ ID NO: 529, and a light chain complementarity determining region 3 (LC CDR3) comprising the amino acid sequence of SEQ ID NO: 571;
(b) a VH comprising a HC CDR1 comprising the amino acid sequence of SEQ ID NO: 1144, a HC CDR2 comprising the amino acid sequence of SEQ ID NO: 1145, and a HC CDR3 comprising the amino acid sequence of SEQ ID NO: 410; and a VL comprising a LC CDR1 comprising the amino acid sequence of SEQ ID NO: 1146, a LC CDR2 comprising the amino acid sequence of SEQ ID NO: 529, and a LC CDR3 comprising the amino acid sequence of SEQ ID NO: 571;
(c) a VH comprising a HC CDR1 comprising the amino acid sequence of SEQ ID NO: 1165, a HC CDR2 comprising the amino acid sequence of SEQ ID NO: 1166, and a HC CDR3 comprising the amino acid sequence of SEQ ID NO: 1167; and a VL comprising a LC CDR1 comprising the amino acid sequence of SEQ ID NO: 473, a LC CDR2 comprising the amino acid sequence of RVS, and a LC CDR3 comprising the amino acid sequence of SEQ ID NO: 571; or
(d) a VH comprising a HC CDR1 comprising the amino acid sequence of SEQ ID NO: 314, a HC CDR2 comprising the amino acid sequence of SEQ ID NO: 341, and a HC CDR3 comprising the amino acid sequence of SEQ ID NO: 410; and a VL comprising a LC CDR1 comprising the amino acid sequence of SEQ ID NO: 1154, a LC CDR2 comprising the amino acid sequence of SEQ ID NO: 529, and a LC CDR3 comprising the amino acid sequence of SEQ ID NO: 571; and
wherein:
(i) the VH comprises the amino acid sequence of SEQ ID NO: 71, or an amino acid sequence at least 86% (e.g., at least 90, 92, 95, 96, 97, 98, or 99%) identical to the amino acid sequence of SEQ ID NO: 71; and/or
(ii) the VL comprises the amino acid sequence of SEQ ID NO: 73, or an amino acid sequence at least 91% (e.g., at least 92, 95, 96, 97, 98, or 99/6) identical to the amino acid sequence of SEQ ID NO: 73.
   81. An antibody that binds to human tau, which comprises a heavy chain variable region (VH) comprising the amino acid sequence of SEQ ID NO: 71, and a light chain variable region (VL) comprising the amino acid sequence of SEQ ID NO: 73.
   82. An antibody that binds to human tau, which comprises:
(a) a heavy chain variable region (VH) comprising a heavy chain complementarity determining region 1 (HC CDR1) comprising the amino acid sequence of SEQ ID NO: 64, a heavy chain complementarity determining region 2 (HC CDR2) comprising the amino acid sequence of SEQ ID NO: 1145, and a heavy chain complementarity determining region 3 (HC CDR3) comprising the amino acid sequence of SEQ ID NO: 1167; and a light chain variable region (VL) comprising a light chain complementarity determining region 1 (LC CDR1) comprising the amino acid sequence of SEQ ID NO: 1146, a light chain complementarity determining region 2 (LC CDR2) comprising the amino acid sequence of SEQ ID NO: 529, and a light chain complementarity determining region 3 (LC CDR3) comprising the amino acid sequence of SEQ ID NO: 571;
(b) a VH comprising a HC CDR1 comprising the amino acid sequence of SEQ ID NO: 1144, a HC CDR2 comprising the amino acid sequence of SEQ ID NO: 1145, and a HC CDR3 comprising the amino acid sequence of SEQ ID NO: 410; and a VL comprising a LC CDR1 comprising the amino acid sequence of SEQ ID NO: 1146, a LC CDR2 comprising the amino acid sequence of SEQ ID NO: 529, and a LC CDR3 comprising the amino acid sequence of SEQ ID NO: 571;
(c) a VH comprising a HC CDR1 comprising the amino acid sequence of SEQ ID NO: 1165, a HC CDR2 comprising the amino acid sequence of SEQ ID NO: 1166, and a HC CDR3 comprising the amino acid sequence of SEQ ID NO: 1167; and a VL comprising a LC CDR1 comprising the amino acid sequence of SEQ ID NO: 473, a LC CDR2 comprising the amino acid sequence of RVS, and a LC CDR3 comprising the amino acid sequence of SEQ ID NO: 571; or
(d) a VH comprising a HC CDR1 comprising the amino acid sequence of SEQ ID NO: 314, a HC CDR2 comprising the amino acid sequence of SEQ ID NO: 341, and a HC CDR3 comprising the amino acid sequence of SEQ ID NO: 410; and a VL comprising a LC CDR1 comprising the amino acid sequence of SEQ ID NO: 1154, a LC CDR2 comprising the amino acid sequence of SEQ ID NO: 529, and a LC CDR3 comprising the amino acid sequence of SEQ ID NO: 571; and
wherein:
(i) the VH comprises 1, 2, 3, 4, 5, 6, 7, 8, 9, 10, 11, 12, 13, 14, 15, 16 or all of: V at position 5; S at position 7; A at position 9; V at position 11; K at position 12; A at position 16; K at position 19; V at position 20; Q at position 43; M at position 48; R at position 67; V at position 68; M at position 70; T at position 76; S at position 77; R at position 87; and/or T at position 91, numbered according to SEQ ID NO: 21; and
(ii) the VL comprises 1, 2, 3, 4, 5, 6, 7, 8, 9, 10, 11, 12, 13, or all of: I at position 2; S at position 11; T at position 14; Q at position 17; P at position 18; Q at position 42; R at position 44; P at position 48; R at position 50; S at position 68; A at position 72; N at position 81; V at position 88; and/or Q at position 105, numbered according to SEQ ID NO: 93.
   83. An antibody that binds to human tau, which comprises:
(a) a heavy chain variable region (VH) comprising a heavy chain complementarity determining region 1 (HC CDR1) comprising the amino acid sequence of SEQ ID NO: 64, a heavy chain complementarity determining region 2 (HC CDR2) comprising the amino acid sequence of SEQ ID NO: 1145, and a heavy chain complementarity determining region 3 (HC CDR3) comprising the amino acid sequence of SEQ ID NO: 1167; and a light chain variable region (VL) comprising a light chain complementarity determining region 1 (LC CDR1) comprising the amino acid sequence of SEQ ID NO: 1146, a light chain complementarity determining region 2 (LC CDR2) comprising the amino acid sequence of SEQ ID NO: 529, and a light chain complementarity determining region 3 (LC CDR3) comprising the amino acid sequence of SEQ ID NO: 571;
(b) a VH comprising a HC CDR1 comprising the amino acid sequence of SEQ ID NO: 1144, a HC CDR2 comprising the amino acid sequence of SEQ ID NO: 1145, and a HC CDR3 comprising the amino acid sequence of SEQ ID NO: 410; and a VL comprising a LC CDR1 comprising the amino acid sequence of SEQ ID NO: 1146, a LC CDR2 comprising the amino acid sequence of SEQ ID NO: 529, and a LC CDR3 comprising the amino acid sequence of SEQ ID NO: 571;
(c) a VH comprising a HC CDR1 comprising the amino acid sequence of SEQ ID NO: 1165, a HC CDR2 comprising the amino acid sequence of SEQ ID NO: 1166, and a HC CDR3 comprising the amino acid sequence of SEQ ID NO: 1167; and a VL comprising a LC CDR1 comprising the amino acid sequence of SEQ ID NO: 473, a LC CDR2 comprising the amino acid sequence of RVS, and a LC CDR3 comprising the amino acid sequence of SEQ ID NO: 571; or
(d) a VH comprising a HC CDR1 comprising the amino acid sequence of SEQ ID NO: 314, a HC CDR2 comprising the amino acid sequence of SEQ ID NO: 341, and a HC CDR3 comprising the amino acid sequence of SEQ ID NO: 410; and a VL comprising a LC CDR1 comprising the amino acid sequence of SEQ ID NO: 1154, a LC CDR2 comprising the amino acid sequence of SEQ ID NO: 529, and a LC CDR3 comprising the amino acid sequence of SEQ ID NO: 571; and
wherein:
(i) the VH comprises the amino acid sequence of SEQ ID NO: 71, or an amino acid sequence at least 86% (e.g., at least 90, 92, 95, 96, 97, 98, or 99%) identical to the amino acid sequence of SEQ ID NO: 71; and/or
(ii) the VL comprises the amino acid sequence of SEQ ID NO: 74, or an amino acid sequence at least 91% (e.g., at least 92, 95, 96, 97, 98, or 99/6) identical to the amino acid sequence of SEQ ID NO: 74.
   84. An antibody that binds to human tau, which comprises a heavy chain variable region (VH) comprising the amino acid sequence of SEQ ID NO: 71, and a light chain variable region (VL) comprising the amino acid sequence of SEQ ID NO: 74.
   85. The antibody of any one of the preceding embodiments, wherein the antibody is a humanized antibody.
   86. The antibody of any one of the preceding embodiments, which is a full length antibody, a bispecific antibody, an Fab, an F(ab′)₂, an Fv, or a single chain Fv fragment (scFv).
   87. The antibody of any one of the preceding embodiments, which comprises a heavy chain constant region selected from IgG1, IgG2, IgG3, IgG4; and/or a light chain constant region of kappa or lambda.
   88. The antibody of any one of the preceding embodiments, which comprises a heavy chain constant region of IgG4 and a light chain constant region of kappa.
   89. The antibody of any one of the preceding embodiments, wherein:
(i) the antibody comprises a human IgG4 constant region, comprising an amino acid other than serine at position 228 according to EU numbering;
(ii) the antibody comprises a human IgG4 constant region, comprising a serine to proline substitution (e.g., mutation) at position 228 according to EU numbering;
(iii) the antibody comprises a heavy chain constant region (e.g., a human IgG4 constant region) comprising the amino acid sequence of SEQ ID NO: 194, or an amino acid sequence at least 85%, 90%, 95%, 96%, 97%, 98%, or 99% identical to SEQ ID NO: 194; and/or
(iv) the antibody comprises a heavy chain constant region (e.g., a human IgG4 constant region), wherein the nucleotide sequence encoding the heavy chain constant region comprises the nucleotide sequence of any one of SEQ ID NOs: 195, 196, 198, or 199, or a nucleotide sequence at least 85%, 90%, 95%, 96%, 97%, 98%, or 99% identical any one of SEQ ID NOs: 195, 196, 198, or 199.
   90. The antibody of any one of embodiments 1-35, wherein the antibody comprises a light chain constant region (e.g., a light chain constant region of kappa), wherein:
(i) the light chain constant region comprises the amino acid sequence of SEQ ID NO: 200, or an amino acid sequence at least 85%, 90%, 95%, 96%, 97%, 98%, or 99% identical to SEQ ID NO: 200; and/or
(ii) the nucleotide sequence encoding the light chain constant region comprises the nucleotide sequence of SEQ ID NO: 201 or 202, or a nucleotide sequence at least 85%, 90%, 95%, 96%, 97%, 98%, or 99% identical to SEQ ID NO: 201 or 202.
   91. The antibody of any one of embodiments 1-90, wherein the antibody comprises a heavy chain comprising the amino acid sequence of any one of SEQ ID NOs: 170-174, or an amino acid sequence at least 85%, 90%, 95%, 96%, 97%, 98%, or 99% identical to any one of SEQ ID NOs: 170-174.
   92. The antibody of any one of embodiments 1-91, wherein the antibody comprises a light chain comprising the amino acid sequence of any one of SEQ ID NOs: 175-179, or an amino acid sequence at least 85%, 90%, 95%, 96%, 97%, 98%, or 99% identical to any one of SEQ ID NOs: 175-179.
   93. The antibody of any one of embodiments 1-92, wherein the antibody comprises:
(i) a heavy chain comprising the amino acid sequence of any one of SEQ ID NOs: 170-174, or an amino acid sequence at least 85%, 90%, 95%, 96%, 97%, 98%, or 99% identical to any one of SEQ ID NOs: 170-174; and
(ii) a light chain comprising the amino acid sequence of any one of SEQ ID NOs: 175-179, or an amino acid sequence at least 85%, 90%, 95%, 96%, 97%, 98%, or 99% identical to any one of SEQ ID NOs: 175-179.
   94. The antibody of any one of embodiments 1-93, wherein the antibody comprises:
(i) a heavy chain comprising the amino acid sequence of any one of SEQ ID NOs: 170 or 172-174, or an amino acid sequence at least 85%, 90%, 95%, 96%, 97%, 98%, or 99% identical to any one of SEQ ID NOs: 170 or 172-174; and
(ii) a light chain comprising the amino acid sequence of any one of SEQ ID NOs: 175-177, or an amino acid sequence at least 85%, 90%, 95%, 96%, 97%, 98%, or 99% identical to any one of SEQ ID NOs: 175-177.
   95. The antibody of any one of the preceding embodiments, which comprises:
(i) a heavy chain comprising the amino acid sequence of SEQ ID NO: 170, or an amino acid sequence at least 85%, 90%, 95%, 96%, 97%, 98%, or 99% identical to SEQ ID NO: 170; and a light chain comprising the amino acid sequence of SEQ ID NO: 175, or an amino acid sequence at least 85%, 90%, 95%, 96%, 97%, 98%, or 99% identical to SEQ ID NO: 175;
(ii) a heavy chain comprising the amino acid sequence of SEQ ID NO: 170, or an amino acid sequence at least 85%, 90%, 95%, 96%, 97%, 98%, or 99% identical to SEQ ID NO: 170; and a light chain comprising the amino acid sequence of SEQ ID NO: 176, or an amino acid sequence at least 85%, 90%, 95%, 96%, 97%, 98%, or 99% identical to SEQ ID NO: 176;
(iii) a heavy chain comprising the amino acid sequence of SEQ ID NO: 170, or an amino acid sequence at least 85%, 90%, 95%, 96%, 97%, 98%, or 99% identical to SEQ ID NO: 170; and a light chain comprising the amino acid sequence of SEQ ID NO: 177, or an amino acid sequence at least 85%, 90%, 95%, 96%, 97%, 98%, or 99% identical to SEQ ID NO: 177;
(iv) a heavy chain comprising the amino acid sequence of SEQ ID NO: 170, or an amino acid sequence at least 85%, 90%, 95%, 96%, 97%, 98%, or 99% identical to SEQ ID NO: 170; and a light chain comprising the amino acid sequence of SEQ ID NO: 178, or an amino acid sequence at least 85%, 90%, 95%, 96%, 97%, 98%, or 99% identical to SEQ ID NO: 178;
(v) a heavy chain comprising the amino acid sequence of SEQ ID NO: 170, or an amino acid sequence at least 85%, 90%, 95%, 96%, 97%, 98%, or 99% identical to SEQ ID NO: 170; and a light chain comprising the amino acid sequence of SEQ ID NO: 179, or an amino acid sequence at least 85%, 90%, 95%, 96%, 97%, 98%, or 99% identical to SEQ ID NO: 179;
(vi) a heavy chain comprising the amino acid sequence of SEQ ID NO: 171, or an amino acid sequence at least 85%, 90%, 95%, 96%, 97%, 98%, or 99% identical to SEQ ID NO: 171; and a light chain comprising the amino acid sequence of SEQ ID NO: 175, or an amino acid sequence at least 85%, 90%, 95%, 96%, 97%, 98%, or 99% identical to SEQ ID NO: 175;
(vii) a heavy chain comprising the amino acid sequence of SEQ ID NO: 171, or an amino acid sequence at least 85%, 90%, 95%, 96%, 97%, 98%, or 99% identical to SEQ ID NO: 171; and a light chain comprising the amino acid sequence of SEQ ID NO: 176, or an amino acid sequence at least 85%, 90%, 95%, 96%, 97%, 98%, or 99% identical to SEQ ID NO: 176;
(viii) a heavy chain comprising the amino acid sequence of SEQ ID NO: 171, or an amino acid sequence at least 85%, 90%, 95%, 96%, 97%, 98%, or 99% identical to SEQ ID NO: 171; and a light chain comprising the amino acid sequence of SEQ ID NO: 177, or an amino acid sequence at least 85%, 90%, 95%, 96%, 97%, 98%, or 99% identical to SEQ ID NO: 177;
(ix) a heavy chain comprising the amino acid sequence of SEQ ID NO: 171, or an amino acid sequence at least 85%, 90%, 95%, 96%, 97%, 98%, or 99% identical to SEQ ID NO: 171; and a light chain comprising the amino acid sequence of SEQ ID NO: 178, or an amino acid sequence at least 85%, 90%, 95%, 96%, 97%, 98%, or 99% identical to SEQ ID NO: 178;
(x) a heavy chain comprising the amino acid sequence of SEQ ID NO: 171, or an amino acid sequence at least 85%, 90%, 95%, 96%, 97%, 98%, or 99% identical to SEQ ID NO: 171; and a light chain comprising the amino acid sequence of SEQ ID NO: 179, or an amino acid sequence at least 85%, 90%, 95%, 96%, 97%, 98%, or 99% identical to SEQ ID NO: 179;
(xi) a heavy chain comprising the amino acid sequence of SEQ ID NO: 172, or an amino acid sequence at least 85%, 90%, 95%, 96%, 97%, 98%, or 99% identical to SEQ ID NO: 172; and a light chain comprising the amino acid sequence of SEQ ID NO: 175, or an amino acid sequence at least 85%, 90%, 95%, 96%, 97%, 98%, or 99% identical to SEQ ID NO: 175;
(xii) a heavy chain comprising the amino acid sequence of SEQ ID NO: 172, or an amino acid sequence at least 85%, 90%, 95%, 96%, 97%, 98%, or 99% identical to SEQ ID NO: 172; and a light chain comprising the amino acid sequence of SEQ ID NO: 176, or an amino acid sequence at least 85%, 90%, 95%, 96%, 97%, 98%, or 99% identical to SEQ ID NO: 176;
(xiii) a heavy chain comprising the amino acid sequence of SEQ ID NO: 172, or an amino acid sequence at least 85%, 90%, 95%, 96%, 97%, 98%, or 99% identical to SEQ ID NO: 172; and a light chain comprising the amino acid sequence of SEQ ID NO: 177, or an amino acid sequence at least 85%, 90%, 95%, 96%, 97%, 98%, or 99% identical to SEQ ID NO: 177;
(xiv) a heavy chain comprising the amino acid sequence of SEQ ID NO: 172, or an amino acid sequence at least 85%, 90%, 95%, 96%, 97%, 98%, or 99% identical to SEQ ID NO: 172; and a light chain comprising the amino acid sequence of SEQ ID NO: 178, or an amino acid sequence at least 85%, 90%, 95%, 96%, 97%, 98%, or 99% identical to SEQ ID NO: 178;
(xv) a heavy chain comprising the amino acid sequence of SEQ ID NO: 172, or an amino acid sequence at least 85%, 90%, 95%, 96%, 97%, 98%, or 99% identical to SEQ ID NO: 172; and a light chain comprising the amino acid sequence of SEQ ID NO: 179, or an amino acid sequence at least 85%, 90%, 95%, 96%, 97%, 98%, or 99% identical to SEQ ID NO: 179;
(xvi) a heavy chain comprising the amino acid sequence of SEQ ID NO: 173, or an amino acid sequence at least 85%, 90%, 95%, 96%, 97%, 98%, or 99% identical to SEQ ID NO: 173; and a light chain comprising the amino acid sequence of SEQ ID NO: 175, or an amino acid sequence at least 85%, 90%, 95%, 96%, 97%, 98%, or 99% identical to SEQ ID NO: 175;
(xvii) a heavy chain comprising the amino acid sequence of SEQ ID NO: 173, or an amino acid sequence at least 85%, 90%, 95%, 96%, 97%, 98%, or 99% identical to SEQ ID NO: 173; and a light chain comprising the amino acid sequence of SEQ ID NO: 176, or an amino acid sequence at least 85%, 90%, 95%, 96%, 97%, 98%, or 99% identical to SEQ ID NO: 176;
(xviii) a heavy chain comprising the amino acid sequence of SEQ ID NO: 173, or an amino acid sequence at least 85%, 90%, 95%, 96%, 97%, 98%, or 99% identical to SEQ ID NO: 173; and a light chain comprising the amino acid sequence of SEQ ID NO: 177, or an amino acid sequence at least 85%, 90%, 95%, 96%, 97%, 98%, or 99% identical to SEQ ID NO: 177;
(xix) a heavy chain comprising the amino acid sequence of SEQ ID NO: 173, or an amino acid sequence at least 85%, 90%, 95%, 96%, 97%, 98%, or 99% identical to SEQ ID NO: 173; and a light chain comprising the amino acid sequence of SEQ ID NO: 178, or an amino acid sequence at least 85%, 90%, 95%, 96%, 97%, 98%, or 99% identical to SEQ ID NO: 178;
(xx) a heavy chain comprising the amino acid sequence of SEQ ID NO: 173, or an amino acid sequence at least 85%, 90%, 95%, 96%, 97%, 98%, or 99% identical to SEQ ID NO: 173; and a light chain comprising the amino acid sequence of SEQ ID NO: 179, or an amino acid sequence at least 85%, 90%, 95%, 96%, 97%, 98%, or 99% identical to SEQ ID NO: 179;
(xxi) a heavy chain comprising the amino acid sequence of SEQ ID NO: 174, or an amino acid sequence at least 85%, 90%, 95%, 96%, 97%, 98%, or 99% identical to SEQ ID NO: 174; and a light chain comprising the amino acid sequence of SEQ ID NO: 175, or an amino acid sequence at least 85%, 90%, 95%, 96%, 97%, 98%, or 99% identical to SEQ ID NO: 175;
(xxii) a heavy chain comprising the amino acid sequence of SEQ ID NO: 174, or an amino acid sequence at least 85%, 90%, 95%, 96%, 97%, 98%, or 99% identical to SEQ ID NO: 174; and a light chain comprising the amino acid sequence of SEQ ID NO: 176, or an amino acid sequence at least 85%, 90%, 95%, 96%, 97%, 98%, or 99% identical to SEQ ID NO: 176;
(xxiii) a heavy chain comprising the amino acid sequence of SEQ ID NO: 174, or an amino acid sequence at least 85%, 90%, 95%, 96%, 97%, 98%, or 99% identical to SEQ ID NO: 174; and a light chain comprising the amino acid sequence of SEQ ID NO: 177, or an amino acid sequence at least 85%, 90%, 95%, 96%, 97%, 98%, or 99% identical to SEQ ID NO: 177;
(xxiv) a heavy chain comprising the amino acid sequence of SEQ ID NO: 174, or an amino acid sequence at least 85%, 90%, 95%, 96%, 97%, 98%, or 99% identical to SEQ ID NO: 174; and a light chain comprising the amino acid sequence of SEQ ID NO: 178, or an amino acid sequence at least 85%, 90%, 95%, 96%, 97%, 98%, or 99% identical to SEQ ID NO: 178; or
(xxv) a heavy chain comprising the amino acid sequence of SEQ ID NO: 174, or an amino acid sequence at least 85%, 90%, 95%, 96%, 97%, 98%, or 99% identical to SEQ ID NO: 174; and a light chain comprising the amino acid sequence of SEQ ID NO: 179, or an amino acid sequence at least 85%, 90%, 95%, 96%, 97%, 98%, or 99% identical to SEQ ID NO: 179.
   96. The antibody of any one of embodiments 1-11, 15-17, 21-23, 26-28, 31-38, 44-49, 52-58, 63-65, 70-72, or 85-95, which comprises a heavy chain comprising the amino acid sequence of SEQ ID NO: 172, or an amino acid sequence at least 85%, 90%, 95%, 96%, 97%, 98%, or 99% identical to SEQ ID NO: 172; and a light chain comprising the amino acid sequence of SEQ ID NO: 176, or an amino acid sequence at least 85%, 90%, 95%, 96%, 97%, 98%, or 99% identical to SEQ ID NO: 176.
   97. The antibody of any one of embodiments 1-11, 15-17, 21-23, 26-28, 31-38, 44-49, 52-58, 63-65, 69-72, or 85-96, which comprises a heavy chain comprising the amino acid sequence of SEQ ID NO: 172 and a light chain comprising the amino acid sequence of SEQ ID NO: 176.
   98. The antibody of any one of embodiments 1-10, 12, 15, 16, 18, 21, 22, 24, 26, 27, 29, 31-37, 41, 44-46, 50, 52-56, 59, 63, 64, 69, 73-75, or 85-95, which comprises a heavy chain comprising the amino acid sequence of SEQ ID NO: 170, or an amino acid sequence at least 85%, 90%, 95%, 96%, 97%, 98%, or 99% identical to SEQ ID NO: 170; and a light chain comprising the amino acid sequence of SEQ ID NO: 175, or an amino acid sequence at least 85%, 90%, 95%, 96%, 97%, 98%, or 99% identical to SEQ ID NO: 175.
   99. The antibody of any one of embodiments 1-10, 13, 15, 16, 19, 21, 22, 24, 26, 27, 29, 31-37, 42, 44-46, 50, 52-56, 60, 63, 64, 67, 69, 76-78, or 85-95, which comprises a heavy chain comprising the amino acid sequence of SEQ ID NO: 173, or an amino acid sequence at least 85%, 90%, 95%, 96%, 97%, 98%, or 99% identical to SEQ ID NO: 173; and a light chain comprising the amino acid sequence of SEQ ID NO: 175, or an amino acid sequence at least 85%, 90%, 95%, 96%, 97%, 98%, or 99% identical to SEQ ID NO: 175.

100. The antibody of any one of embodiments 1-10, 14-16, 20-23, 31-37, 43-49, 52-56, 51, 63, 64, 68, 69, 79-81, 85-95, which comprises a heavy chain comprising the amino acid sequence of SEQ ID NO: 174, or an amino acid sequence at least 85%, 90%, 95%, 96%, 97%, 98%, or 99% identical to SEQ ID NO: 174; and a light chain comprising the amino acid sequence of SEQ ID NO: 176, or an amino acid sequence at least 85%, 90%, 95%, 96%, 97%, 98%, or 99% identical to SEQ ID NO: 176.

101. The antibody of any one of embodiments 1-10, 14-16, 20-22, 25-27, 30-37, 43-46, 51-56, 62-64, 68, 69, 82-95, which comprises a heavy chain comprising the amino acid sequence of SEQ ID NO: 174, or an amino acid sequence at least 85%, 90%, 95%, 960%, 97%, 98%, or 99% identical to SEQ ID NO: 174; and a light chain comprising the amino acid sequence of SEQ ID NO: 177, or an amino acid sequence at least 85%, 90%, 95%, 96%, 97%, 98%, or 99% identical to SEQ ID NO: 177.
102. The antibody of any one of the preceding embodiments, which comprises:
(i) a heavy chain, wherein the nucleotide sequence encoding the heavy chain comprises the nucleotide sequence of any one of SEQ ID NOs: 180-184, or an nucleotide acid sequence at least 85%, 90%, 95%, 96%, 97%, 98%, or 99% identical to any one of SEQ ID NOs: 180-184; and/or
(ii) a light chain, where the nucleotide sequence encoding the light chain comprises the nucleotide sequence of any one of SEQ ID NOs: 185-189, or an nucleotide acid sequence at least 85%, 90%, 95%, 96%, 97%, 98%, or 99% identical to any one of SEQ ID NOs: 185-189.
   103. The antibody of any one of the preceding embodiments, which comprises:
(i) a heavy chain, wherein the nucleotide sequence encoding the heavy chain comprises the nucleotide sequence of any one of SEQ ID NOs: 180 or 182-184, or an nucleotide acid sequence at least 85%, 90%, 95%, 96%, 97%, 98%, or 99% identical to any one of SEQ ID NOs: 180 or 182-184; and/or
(ii) a light chain, where the nucleotide sequence encoding the light chain comprises the nucleotide sequence of any one of SEQ ID NOs: 185-187, or an nucleotide acid sequence at least 85%, 90%, 95%, 96%, 97%, 98%, or 99% identical to any one of SEQ ID NOs: 185-187.
   104. The antibody of any one of embodiments 1-11, 15-17, 21-23, 26-28, 31-38, 44-49, 52-58, 63-65, 70-72, 85-98, 102, or 103, which comprises:
(i) a heavy chain, wherein the nucleotide sequence encoding the heavy chain comprises the nucleotide sequence of SEQ ID NO: 182, or an nucleotide acid sequence at least 85%, 90%, 95%, 96%, 97%, 98%, or 99% identical to SEQ ID NO: 182; and/or
(ii) a light chain, where the nucleotide sequence encoding the light chain comprises the nucleotide sequence of SEQ ID NO: 186, or an nucleotide acid sequence at least 85%, 90%, 95%, 96%, 97%, 98%, or 99% identical to SEQ ID NO: 186.
   105. The antibody of any one of embodiments 1-10, 12, 15, 16, 18, 21, 22, 24, 26, 27, 29, 31-37, 41, 44-46, 50, 52-56, 59, 63, 64, 69, 73-75, 85-95, 98, 102, or 103, which comprises:
(i) a heavy chain, wherein the nucleotide sequence encoding the heavy chain comprises the nucleotide sequence of SEQ ID NO: 180, or an nucleotide acid sequence at least 85%, 90%, 95%, 96%, 97%, 98%, or 99% identical to SEQ ID NO: 180; and/or
(ii) a light chain, where the nucleotide sequence encoding the light chain comprises the nucleotide sequence of SEQ ID NO: 185, or an nucleotide acid sequence at least 85%, 90%, 95%, 96%, 97%, 98%, or 99% identical to SEQ ID NO: 185.
   106. The antibody of any one of embodiments 1-10, 13, 15, 16, 19, 21, 22, 24, 26, 27, 29, 31-37, 42, 44-46, 50, 52-56, 60, 63, 64, 67, 69, 76-78, 85-95, 99, 102, or 103, which comprises:
(i) a heavy chain, wherein the nucleotide sequence encoding the heavy chain comprises the nucleotide sequence of SEQ ID NO: 183, or an nucleotide acid sequence at least 85%, 90%, 95%, 96%, 97%, 98%, or 99% identical to SEQ ID NO: 183; and/or
(ii) a light chain, where the nucleotide sequence encoding the light chain comprises the nucleotide sequence of SEQ ID NO: 185, or an nucleotide acid sequence at least 85%, 90%, 95%, 96%, 97%, 98%, or 99% identical to SEQ ID NO: 185.
   107. The antibody of any one of embodiments 1-10, 14-16, 20-23, 25-27, 30-37,43-49, 51-56, 51, 62-65, 68, 69, 79-95, or 100-103, which comprises:
(i) a heavy chain, wherein the nucleotide sequence encoding the heavy chain comprises the nucleotide sequence of SEQ ID NO: 184, or an nucleotide acid sequence at least 85%, 90%, 95%, 96%, 97%, 98%, or 99% identical to SEQ ID NO: 184; and/or
(ii) a light chain, where the nucleotide sequence encoding the light chain comprises the nucleotide sequence of SEQ ID NO: 186 or 187, or an nucleotide acid sequence at least 85%, 90%, 95%, 96%, 97%, 98%, or 99% identical to SEQ ID NO: 186 or 187.
   108. The antibody of any one of the preceding embodiments, wherein the antibody binds the C-terminus of a tau protein, e.g., residues 409-436 numbered according to SEQ ID NO: 920.
   109. The antibody of any one of the preceding embodiments, wherein the antibody binds a phosphorylated residue of a tau protein.
   110. The antibody of any one of the preceding embodiments, wherein the antibody binds phosphorylated serine at position 422 (e.g., pS422), numbered according to SEQ ID NO: 920.
   111. The antibody of any one of the preceding embodiments, wherein the antibody preferentially binds pathological tau (e.g., PHF-tau or ePHF), compared to wild-type tau, e.g., as measured by an assay, e.g., an ELISA assay, an SPR assay or a Biacore assay, e.g., an assay as described in Example 1 or Example 8.
   112. The antibody of any one of the preceding embodiments, which reduces, e.g., inhibits, aggregation of tau.
   113. The antibody of any one of the preceding embodiments, which binds an epitope comprising a region formed by a complex of at least two tau proteins, e.g., a tau dimer.
   114. The antibody of any one of the preceding embodiments which has one, two, three, four, five, or all of the following properties:
(i) is capable of binding to iPHF with an affinity of at least about 24-50 pM (e.g., an affinity of at least about 24, 29, 30, 32, 33, 35, 37, 39, 41, 43, 45, 47, or 48 pM), e.g., when measured by an assay (e.g., an SPR or Biacore assay), e.g., an assay as described in Example 8;
(ii) is capable of binding to phosphorylated serine at position 422 (e.g., pS422) of human tau, numbered according to SEQ ID NO: 920 (e.g., a peptide comprising the amino acid sequence of SEQ ID NO: 33), with an affinity of at least about 29-77 pM (e.g., an affinity of at least about 29, 33, 35, 38, 39, 40, 41, 43, 44, 45, 47, 50, 53, 54, 55, 60, 65, 70, 75, 76, or 77 pM), e.g., when measured by an assay (e.g., an SPR or Biacore assay), e.g., an assay as described in Example 8;
(iii) is capable of binding to ePHF with an affinity of at least about 0.08-0.2 nM (e.g., at least about 0.08, 0.085, 0.09, 0.095, 0.1, 0.11, 0.12, 0.13, 0.14, 0.15, 0.16, 0.17, 0.18, 0.19 or 0.2 nM), e.g., when measured by an assay (e.g., an ELISA assay), e.g., an assay as described in Example 8; (iv) demonstrates low polyspecificity, e.g., a BVP score of at least about 1.6-6 (e.g., a BVP score of at least about 1.6, 1.7, 1.8, 2, 2.2, 2.5, 2.7, 3, 3.2, 3.5, 3.7, 3.9, 4, 4.5, 5, 5.5, or 6), e.g., when measured by an assay (e.g., a BVP ELISA assay), e.g., an assay as described in Example 8;
(v) is capable of binding pathological tau, e.g., as measured by an assay, e.g., an IHC assay, e.g., an assay as described in Example 8; and/or
(vi) preferentially binds pathological tau (e.g., PHF-tau or ePHF), compared to wild-type tau, e.g., as measured by an assay, e.g., an ELISA assay, an SPR assay or a Biacore assay, e.g., an assay as described in Example 8.
   115. The antibody of any one of the preceding embodiments wherein the antibody demonstrates a low immunogenicity risk (e.g., having a response rate of no more than about 55% (e.g., no more than about 50, 46, 48, 30, 24, or 20%) of donors (e.g., in PBMC cells isolated from the donor), e.g., as measured by an assay, e.g., a T-cell proliferation assay, e.g., an assay as described in Example 8.
   116. An antibody that competes for binding to tau with the antibody of any one of the preceding embodiments.
   117. An antibody that binds to the same epitope as, substantially the same epitope as, or an epitope that overlaps with, the epitope of the antibody of any one of the preceding embodiments.
   118. A nucleic acid encoding the antibody of any one of the preceding claims.
   119. The nucleic acid of embodiment 118, which encodes:
(i) a VH, wherein the nucleotide sequence encoding the VH comprises the nucleotide sequence of any one of SEQ ID NOs: 156-160, or a nucleotide sequence at least 86% (e.g., at least 87%, 90%, 95%, 96%, 97%, 98%, or 99%) identical thereto; and/or
(ii) a VL, wherein the nucleotide sequence encoding the VL comprises the nucleotide sequence of any one of SEQ ID NOs: 161-165; or a nucleotide sequence at least 86% (e.g., at least 91%, 95%, 96%, 97%, 98%, or 99%) identical thereto.
   120. The nucleic acid of embodiment 118 or 119, which encodes:
(i) a VH, wherein the nucleotide sequence encoding the VH comprises the nucleotide sequence of any one of SEQ ID NOs: 156 or 158-160, or a nucleotide sequence at least 86% (e.g., at least 87%, 90%, 95%, 96%, 97%, 98%, or 99%) identical thereto; and/or
(ii) a VL, wherein the nucleotide sequence encoding the VL comprises the nucleotide sequence of any one of SEQ ID NOs: 161-163; or a nucleotide sequence at least 86% (e.g., at least 91%, 95%, 96%, 97%, 98%, or 99%) identical thereto.
   121. The nucleic acid of any one of embodiments 118-120, which encodes:
(i) a heavy chain, wherein the nucleotide sequence encoding the heavy chain comprises the nucleotide sequence of any one of SEQ ID NOs: 180-184, or a nucleotide sequence at least 86% (e.g., at least 87%, 90%, 95%, 96%, 97%, 98%, or 99%) identical thereto; and/or
(ii) a light chain, wherein the nucleotide sequence encoding the light chain comprises the nucleotide sequence of any one of SEQ ID NOs: 185-189; or a nucleotide sequence at least 86% (e.g., at least 91%, 95%, 96%, 97%, 98%, or 99%) identical thereto.
   122. The nucleic acid of any one of embodiments 118-121, which encodes:
(i) a heavy chain, wherein the nucleotide sequence encoding the heavy chain comprises the nucleotide sequence of any one of SEQ ID NOs: 180 or 182-184, or a nucleotide sequence at least 86% (e.g., at least 87%, 90%, 95%, 96%, 97%, 98%, or 99%) identical thereto; and/or
(ii) a light chain, wherein the nucleotide sequence encoding the light chain comprises the nucleotide sequence of any one of SEQ ID NOs: 185-187; or a nucleotide sequence at least 86% (e.g., at least 91%, 95%, 96%, 97%, 98%, or 99%) identical thereto.
   123. The nucleic acid of any one of embodiments 118-122, which encodes:
(i) a VH, wherein the nucleotide sequence encoding the VH comprises the nucleotide sequence of SEQ ID NO: 158, or a nucleotide sequence at least 86% (e.g., at least 87%, 90%, 95%, 96%, 97%, 98%, or 99%) identical thereto; and a VL wherein the nucleotide sequence encoding the VL comprises the nucleotide sequence of SEQ ID NO: 162; or a nucleotide sequence at least 86% (e.g., at least 91%, 95%, 96%, 97%, 98%, or 99%) identical thereto; and/or
(ii) a heavy chain, wherein the nucleotide sequence encoding the heavy chain comprises the nucleotide sequence of SEQ ID NO: 182, or a nucleotide sequence at least 86% (e.g., at least 87%, 90%, 95%, 96%, 97%, 98%, or 99%) identical thereto; and a light chain, wherein the nucleotide sequence encoding the light chain comprises the nucleotide sequence of SEQ ID NO: 186; or a nucleotide sequence at least 86% (e.g., at least 91%, 95%, 96%, 97%, 98%, or 99%) identical thereto.
   124. The nucleic acid of any one of embodiments 118-123, which is codon optimized.
   125. An antibody encoded by the nucleic acid of any one of embodiments 118-124.
   126. The antibody of any one of embodiments 1-117 or 125 or the nucleic acid of any one of embodiments 118-124, which is isolated, e.g., recombinant.
   127. A vector comprising the nucleic acid of any one of embodiments 118-124 or 126, or a nucleic acid encoding the antibody of any one of embodiments 1-117, 125 or 126.
   128. A host cell comprising the nucleic acid of any one of embodiments 118-124 or 126, a nucleic acid encoding the antibody of any one of embodiments 1-117, 125, or 126, the antibody of any one of embodiments 1-117, 125, or 126, or the vector of embodiment 127.
   129. The host cell of embodiment 128, wherein the host cell is an insect cell, a bacterial cell, or a mammalian cell.
   130. A method of producing an antibody, the method comprising culturing the host cell of embodiment 128 or 129, under conditions suitable for gene expression.
   131. An isolated nucleic acid encoding a payload, wherein the encoded payload comprises the antibody of any one of embodiments 1-117, 125 or 126.
   132. The nucleic acid of embodiment 131, further encoding a signal sequence, optionally wherein the nucleotide sequence encoding the signal sequence comprises the nucleotide sequence of any of the signal sequences listed in Table 14, or a nucleotide sequence with at least 95% sequence identity thereto.
   133. The nucleic acid of embodiment 131 or 132, further encoding a second signal sequence, optionally wherein the nucleotide sequence encoding the signal sequence comprises the nucleotide sequence of any of the signal sequences listed in Table 14, or a nucleotide sequence with at least 95% sequence identity thereto.
   134. The nucleic acid of any one of embodiments 131-133, wherein the:
(i) the nucleotide sequence encoding the signal sequence is located 5′ relative to the nucleotide sequence encoding the VH; and/or
(ii) the nucleotide sequence encoding the signal sequence is located 5′ relative to the nucleotide sequence encoding the VL.
   135. The nucleic acid of any one of embodiments 131-134, wherein the sequences of the encoded VH and VL are connected directly, e.g., without a linker.
   136. The nucleic acid of any one of embodiments 131-135, wherein the sequences of the encoded VH and VL are connected via a linker.
   137. The nucleic acid of embodiment 136, wherein the linker comprises the nucleotide sequence of any of the linker sequences provided in Table 15, or a nucleotide sequence with at least 95% sequence identity thereto.
   138. The nucleic acid of any one of embodiments 131-137, wherein the encoded payload is a full length antibody, a bispecific antibody, an Fab, an F(ab′)₂, an Fv, or a single chain Fv fragment (scFv).
   139. A viral genome comprising a promoter operably linked to the nucleic acid encoding a payload comprising the antibody of any one of embodiments 1-117, 125, or 126; or the nucleic acid of any one of embodiments 131-138.
   140. The viral genome of embodiment 139, wherein the promoter:
(i) is chosen from human elongation factor 1α-subunit (EF1α), cytomegalovirus (CMV) immediate-early enhancer and/or promoter, chicken β-actin (CBA) and its derivative CAG, β glucuronidase (GUSB), or ubiquitin C (UBC), neuron-specific enolase (NSE), platelet-derived growth factor (PDGF), platelet-derived growth factor B-chain (PDGF-β), intercellular adhesion molecule 2 (ICAM-2), synapsin (Syn), methyl-CpG binding protein 2 (MeCP2), Ca2+/calmodulin-dependent protein kinase II (CaMKII), metabotropic glutamate receptor 2 (mGluR2), neurofilament light (NFL) or heavy (NFH), β-globin minigene nβ2, preproenkephalin (PPE), enkephalin (Enk) and excitatory amino acid transporter 2 (EAAT2), glial fibrillary acidic protein (GFAP), myelin basic protein (MBP), or a fragment, e.g., a truncation, or a functional variant thereof; and/or
(ii) comprises the nucleotide sequence of any of the promoter sequences provided in Table 11, or a nucleotide sequence at least 95% identical thereto.
   141. The viral genome of embodiment 139 or 140, which further comprises an enhancer, optionally wherein the enhancer is a CMV immediate-early (CMVie) enhancer.
   142. The viral genome of any one of embodiments 139-141, which further comprises a polyadenylation (polyA) signal region.
   143. The viral genome of embodiment 142, wherein the polyA signal region comprises the nucleotide sequence of any of SEQ ID NO: 1134-1136, or a nucleotide sequence with at least 95% identity thereto.
   144. The viral genome of any one of embodiments 139-143, further comprising an inverted terminal repeat (ITR) sequence.
   145. The viral genome of embodiment 144, wherein:
(i) the ITR sequence is positioned 5′ relative to the encoded payload; and/or
(ii) the ITR sequence is positioned 3′ relative to the encoded payload.
   146. The viral genome of any one of embodiments 139-145, which comprises an ITR sequence positioned 5′ relative to the encoded payload and an ITR sequence positioned 3′ relative to the encoded payload.
   147. The viral genome of any one of embodiments 139-146, wherein the ITR sequence comprises a nucleotide sequence of any one of SEQ ID NOs: 1035-1038, or a nucleotide sequence with at least 80%, 85%, 90%, or 95% sequence identity thereto.
   148. The viral genome of any one of embodiments 139-147, further comprising an intron region.
   149. The viral genome of embodiment 148, wherein the intron region comprises a nucleotide sequence of any of the intron regions listed in Table 13, or a nucleotide sequence with at least 95% identity thereto.
   150. The viral genome of any one of embodiments 139-149, comprising at least one, two, or three intron regions.
   151. The viral genome of any one of embodiments 139-150, further comprising an exon region.
   152. The viral genome of embodiment 151, wherein the exon region comprises the nucleotide sequence of any of the exon sequences in Table 12, or a nucleotide sequence with at least 95% identity thereto.
   153. The viral genome of any one of embodiments 139-152, comprising at least one, two, or three exon regions.
   154. The viral genome of any one of embodiments 139-153, which further comprises a Kozak sequence, optionally wherein the Kozak sequence comprises the nucleotide sequence of GCCGCCACCATG (SEQ ID NO: 1079) or GAGGAGCCACC (SEQ ID NO: 1089).
   155. The viral genome of any one of embodiments 139-154, which further comprises a nucleotide sequence encoding a miR binding site, e.g., a miR binding site that modulates, e.g., reduces, expression of the payload encoded by the viral genome in a cell or tissue where the corresponding miRNA is expressed.
   156. The viral genome of embodiment 155, which comprises at least 1-5 copies of an encoded miR binding site, e.g., at least 1, 2, 3, 4, or 5 copies.
   157. The viral genome of any one of embodiments 139-156, which comprises at least 3 copies of an encoded miR binding sites, optionally wherein all three copies comprise the same miR binding site, or at least one, two, or all of the copies comprise a different miR binding site.
   158. The viral genome of any one of embodiments 139-157, which comprises at least 4 copies of an encoded miR binding site, optionally wherein all four copies comprise the same miR binding site, or at least one, two, three, or all of the copies comprise a different miR binding site.
   159. The viral genome of any one of embodiments 155-158, wherein the encoded miR binding site comprises a miR122 binding site, a miR183 binding site, a miR-142-3p, or a combination thereof, optionally wherein:
(i) the encoded miR122 binding site comprises the nucleotide sequence of SEQ ID NO: 1029, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 1029;
(ii) the encoded miR183 binding site comprises the nucleotide sequence of SEQ ID NO: 1032, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 1032; and/or
(iii) the encoded miR-142-3p binding site comprises the nucleotide sequence of SEQ ID NO: 1031, or a nucleotide sequence substantially identical (e.g., having at least 70%, 75%, 80%, 85%, 90%, 92%, 95%, 97%, 98%, or 99% sequence identity) thereto; or a nucleotide sequence having at least one, two, three, four, five, six, or seven modifications, but no more than ten modifications of SEQ ID NO: 1031.
   160. The viral genome of any one of embodiments 139-159, which is single stranded or self complementary.
   161. The viral genome of any one of embodiments 139-160, which further comprises:
(i) a nucleotide sequence encoding a Rep protein, e.g., a non-structural protein, wherein the Rep protein comprises a Rep78 protein, a Rep68, Rep52 protein, and/or a Rep40 protein; or
(ii) a second nucleic acid comprising a nucleotide sequence encoding a Rep protein, e.g., a non-structural protein, wherein the Rep protein comprises a Rep78 protein, a Rep68, Rep52 protein, and/or a Rep40 protein.
   162. The viral genome of embodiment 161, wherein the Rep78 protein, the Rep68 protein, the Rep52 protein, and/or the Rep40 protein are encoded by at least one Rep gene.
   163. The viral genome of any one of embodiments 139-162, which further comprises:
(i) a nucleotide sequence encodes a capsid protein, e.g., a structural protein, wherein the capsid protein comprises a VP1 polypeptide, a VP2 polypeptide, and/or a VP3 polypeptide; or
(ii) a second nucleic acid comprising a nucleotide sequence encodes a capsid protein, e.g., a structural protein, wherein the capsid protein comprises a VP1 polypeptide, a VP2 polypeptide, and/or a VP3 polypeptide.
   164. The viral genome of embodiment 163, wherein the VP1 polypeptide, the VP2 polypeptide, and/or the VP3 polypeptide are encoded by at least one Cap gene.
   165. A vector comprising the viral genome of any one embodiments 139-164.
   166. An isolated, e.g., recombinant AAV particle comprising:
(i) a capsid protein, and,
(ii) the nucleic acid of any one of embodiments 131-138, or the viral genome of any one of embodiments 139-164.
   167. The isolated AAV particle of embodiment 166, wherein:
(i) the capsid protein comprises the amino acid sequence of SEQ ID NO: 1003, or an amino acid sequence with at least 80% (e.g., at least about 85, 90, 95, 96, 97, 98, or 99%) sequence identity thereto;
(ii) the capsid protein comprises an amino acid sequence having at least one, two or three modifications but not more than 30, 20 or 10 modifications of the amino acid sequence of SEQ ID NO: 1003;
(iii) the capsid protein comprises the amino acid sequence of SEQ ID NO: 1011, or an amino acid sequence with at least 80% (e.g., at least about 85, 90, 95, 96, 97, 98, or 99%) sequence identity thereto;
(iv) the capsid protein comprises an amino acid sequence having at least one, two or three modifications but not more than 30, 20 or 10 modifications of the amino acid sequence of SEQ ID NO: 1011;
(v) the capsid protein comprises an amino acid sequence encoded by the nucleotide sequence of SEQ ID NO: 1002, or a sequence with at least 80% (e.g., at least about 85, 90, 95, 96, 97, 98, or 99%) sequence identity thereto; and/or
(vi) the nucleotide sequence encoding the capsid protein comprises the nucleotide sequence of SEQ ID NO: 1002, or a sequence with at least 80% (e.g., at least about 85, 90, 95, 96, 97, 98, or 99%) sequence identity thereto.
   168. The isolated AAV particle of embodiment 166 or 167, wherein the capsid protein comprises:
(i) an amino acid substitution at position K449, e.g., a K449R substitution, numbered according to SEQ ID NO:1003;
(ii) an insert comprising the amino acid sequence of TLAVPFK (SEQ ID NO: 1151), optionally wherein the insert is present immediately subsequent to position 588, relative to a reference sequence numbered according to SEQ ID NO:1003;
(iii) an amino acid other than “A” at position 587 and/or an amino acid other than “Q” at position 588, numbered according to SEQ ID NO: 1003; and/or
(iv) the amino acid substitution of A587D and/or Q588G, numbered according to SEQ ID NO: 1003.
   169. The AAV particle of any one of embodiments 166-168, wherein the capsid protein comprises (i) the amino acid substitution of K449R numbered according to SEQ ID NO: 1003; and (ii) an insert comprising the amino acid sequence of TLAVPFK (SEQ ID NO: 1151), optionally wherein the insert is present immediately subsequent to position 588 of SEQ ID NO: 1003.
   170. The AAV particle of any one of embodiments 166-169, wherein the capsid protein comprises (i) the amino acid substitution of K449R numbered according to SEQ ID NO: 1003; (ii) an insert comprising the amino acid sequence of TLAVPFK (SEQ ID NO: 1151), optionally wherein the insert is present immediately subsequent to position 588, relative to a reference sequence numbered according to SEQ ID NO: 1003; and (iii) the amino acid substitutions of A587D and Q588G, numbered according to SEQ ID NO: 1003.
   171. The AAV particle of any one of embodiments 166-169, wherein the capsid protein comprises (i) an insert comprising the amino acid sequence of TLAVPFK (SEQ ID NO: 1151), optionally wherein the insert is present immediately subsequent to position 588, relative to a reference sequence numbered according to SEQ ID NO: 1003; and (ii) the amino acid substitutions of A587D and Q588G, numbered according to SEQ ID NO: 1003.
   172. The AAV particle of any one of embodiments 166-171, wherein the capsid protein comprises a VOY101, VOY201, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/G2A3 (G2A3), AAVG2B4 (G2B4), AAVG2B5 (G2B5), AAVPHP.N (PHP.N), PHP.S, AAV1, AAV2, AAV2 variant, AAV2/3 variant, AAV4, AAV5, AAV6, AAV7, AAV8, AAV9.47, AAV9(hu14), AAV9, AAV9 K449R, AAV10, AAV11, AAV12, AAVrh8, AAVrh10, AAVDJ, AAVDJ8, or AAV2G9 capsid protein, or a functional variant thereof.
   173. The AAV particle of any one of embodiments 166-172, wherein the capsid protein comprises an AAV5 capsid protein or variant thereof or an AAV9 capsid protein or variant thereof.
   174. The AAV particle of any one of embodiments 166-173, wherein the capsid protein comprises:
(i) the amino acid sequence of SEQ ID NO: 1023, or an amino acid sequence substantially identical (e.g., having at least 70%, 75%, 80%, 85%, 90%, 92%, 95%, 97%, 98%, or 99% sequence identity) thereto;
(ii) an amino acid sequence comprising at least one, two, or three modifications but no more than 30, 20, or 10 modifications, e.g., substitutions, relative to the amino acid sequence of SEQ ID NO: 1023; or,
(iii) an amino acid sequence encoded by the nucleotide sequence of SEQ ID NO: 1022 or a nucleotide sequence substantially identical (e.g., having at least 70%, 75%, 80%, 85%, 90%, 92%, 95%, 97%, 98%, or 99% sequence identity) thereto.
   175. The AAV particle of embodiment 174, wherein the nucleotide sequence encoding the capsid protein comprises the nucleotide sequence of SEQ ID NO: 1022, or a nucleotide sequence substantially identical (e.g., having at least 70%, 75%, 80%, 85%, 90%, 92%, 95%, 97%, 98%, or 99% sequence identity) thereto.
   176. A host cell comprising the nucleic acid of any one of embodiments 131-138, the viral genome of any one of embodiments 139-164, or the AAV particle of any one of embodiments 166-175, optionally wherein the host cell is an insect cell, a bacterial cell or a mammalian cell.
   177. A nucleic acid encoding the viral genome of any one of embodiments 139-164, and a backbone region suitable for replication of the viral genome in a cell, e.g., a bacterial cell (e.g., wherein the backbone region comprises one or both of a bacterial origin of replication and a selectable marker).
   178. A method of making a viral genome, the method comprising:
(i) providing the nucleic acid molecule comprising the viral genome of any one of embodiments 139-164; and
(ii) excising the viral genome from the backbone region, e.g., by cleaving the nucleic acid molecule at upstream and downstream of the viral genome.
   179. A method of making an isolated, e.g., recombinant, AAV particle, the method comprising
(i) providing a host cell comprising the viral genome of embodiment 176; and
(ii) incubating the host cell under conditions suitable to enclose the viral genome in a capsid protein;
thereby making the isolated AAV particle.
   180. The method of embodiment 179, further comprising, prior to step (i), introducing a first nucleic acid molecule comprising the viral genome into the host cell.
   181. The method of any one of embodiments 178-180, wherein the host cell comprises a second nucleic acid encoding a capsid protein.
   182. The method of embodiment 181, wherein the second nucleic acid molecule is introduced into the host cell prior to, concurrently with, or after the first nucleic acid molecule.
   183. A pharmaceutical composition comprising the antibody of any one of embodiments 1-117, 125, or 126, an AAV particle of any one of embodiments 166-175, or an AAV particle comprising the viral genome of any one of embodiments 139-164, or the isolated nucleic acid of any one of embodiments 131-138, and a pharmaceutically acceptable excipient.
   184. A method of delivering an exogenous antibody that binds to tau, to a subject, comprising administering an effective amount of the pharmaceutical composition of embodiment 183, the antibody of any one of embodiments 1-117, 125, or 126, an AAV particle of any one of embodiments 166-175, or an AAV particle comprising the viral genome of any one of embodiments 139-164, or the isolated nucleic acid of any one of embodiments 131-138.
   185. The method of embodiment 184, wherein the subject has, has been diagnosed with having, or is at risk of having a disease associated with expression of tau, e.g., aberrant tau expression.
   186. The method of embodiment 184 or 185, wherein the subject has, has been diagnosed with having, or is at risk of having a neurological, e.g., neurodegenerative disorder, mild cognitive impairment, or a traumatic brain injury (TBI).
   187. The method of any one of embodiments 184-186, wherein the subject has, has been diagnosed with having, or is at risk of having a tauopathy.
   188. A method of treating a subject having or diagnosed with having a disease associated with expression of tau comprising administering to the subject an effective amount of the pharmaceutical composition of embodiment 183, the antibody of any one of embodiments 1-117, 125, or 126, an AAV particle of any one of embodiments 166-175, or an AAV particle comprising the viral genome of any one of embodiments 139-164, or the isolated nucleic acid of any one of embodiments 131-138.
   189. A method of treating a subject having or diagnosed with having a neurological, e.g., neurodegenerative disorder, or a traumatic brain injury (TBI), comprising administering to the subject an effective amount of the pharmaceutical composition of embodiment 183, the antibody of any one of embodiments 1-117, 125, or 126, an AAV particle of any one of embodiments 166-175, or an AAV particle comprising the viral genome of any one of embodiments 139-164, or the isolated nucleic acid of any one of embodiments 131-138.
   190. A method of treating a subject having or diagnosed with having a tauopathy comprising administering to the subject an effective amount of the pharmaceutical composition of embodiment 183, the antibody of any one of embodiments 1-117, 125, or 126, an AAV particle of any one of embodiments 166-175, or an AAV particle comprising the viral genome of any one of embodiments 139-164, or the isolated nucleic acid of any one of embodiments 131-138.
   191. The method of any one of embodiments 185-190, wherein the disease associated with Tau expression, the neurological disorder, or the tauopathy comprises AD, FTDP-17, FTLD, FTD, CTE, PSP, Down's syndrome, Pick's disease, CBD, Corticobasal syndrome, ALS, Prion diseases, CJD, Multiple system atrophy, mild cognitive impairment, Tangle-only dementia, or Progressive subcortical gliosis.
   192. The method of any one of embodiments 188-191, where treating comprises prevention of progression of the disease in the subject.
   193. The method of any one of embodiments 184-192, wherein the subject is a human.
   194. The method of any one of embodiments 184-193, wherein the antibody or the AAV particle is administered to the subject intravenously, intramuscularly, via intraparenchymal administration, intracerebroventricularly, via intra-cistema magna (ICM) injection, intrathecally, via focused ultrasound (FUS), e.g., coupled with the intravenous administration of microbubbles (FUS-MB), or MRI-guided FUS coupled with intravenous administration.
   195. The method of any one of embodiments 184-194, wherein the antibody or the AAV particle is administered to the subject intravenously.
   196. The method of any one of embodiments 184-194, wherein the antibody or the AAV particle is administered to the subject via intra-cisterna magna injection (ICM).
   197. The method of any one of embodiments 184-196, further comprising evaluating, e.g., measuring, the level of antibodies generated in a subject, e.g., in a cell or tissue of the subject.
   198. The method of any one of embodiments 184-197, wherein the administration results in the generation of antibodies, e.g., 0.001 μg/mL to 100 mg/mL of antibodies, in the subject, e.g., in a cell or tissue of the subject.
   199. The method of embodiment 198, wherein the cell is a neuronal cell.
   200. The method of embodiment 198, wherein the tissue is a central nervous system tissue, e.g., a brain tissue.
   201. The method of any one of embodiments 184-200, further comprising performing a blood test, an imaging test (e.g., a PET scan or a PET scan in combination with biomarker, e.g., serum biomarker staining), a CNS biopsy sample, or an aqueous cerebral spinal fluid biopsy.
   202. The method of any one of embodiments 197-201, wherein measuring the level of antibodies is performed prior to, during, or subsequent to treatment with the antibody or AAV particle.
   203. The method of any one of embodiments 184-202, wherein the subject has a level of antibodies that is a greater than a reference level, e.g., a subject that has not received treatment with the antibody or the AAV particle, e.g., has not been administered the antibody or the AAV particle.
   204. The method of any one of embodiments 184-203, further comprising administration of an additional therapeutic agent and/or therapy suitable for treatment or prevention of a disorder associated with tau expression, a neurological, e.g., neurodegenerative, disorder.
   205. The method of embodiment 204, wherein the additional therapeutic agent and/or therapy comprises a cholinesterase inhibitor (e.g., donepezil, rivastigmine, and/or galantamine), an N-methyl D-aspartate (NMDA) antagonist (e.g., memantine), an antipsychotic drug, an anti-anxiety drug, an anticonvulsant, a dopamine agonist (e.g., pramipexole, ropinirole, rotigotine, and/or apomorphine), an MAO B inhibitor (e.g., selegiline, rasagiline, and/or safinamide), catechol O-methyltransferase (COMT) inhibitors (entacapone, opicapone, and/or tolcapone), anticholinergics (e.g., benztropine and/or trihexyphenidyl), amantadine, carbidopa-levodopa, deep brain simulation (DBS), or a combination thereof.
   206. The antibody of any one of embodiments 1-117, 125, or 126, the pharmaceutical composition of embodiment 183, or the AAV particle of any one of embodiments 166-175, for use in the manufacture of a medicament.
   207. The antibody of any one of embodiments 1-117, 125, or 126, the pharmaceutical composition of embodiment 183, or the AAV particle of any one of embodiments 166-175, for use in the treatment of a disease associated with tau expression.
   208. The antibody of any one of embodiments 1-117, 125, or 126, the pharmaceutical composition of embodiment 183, or the AAV particle of any one of embodiments 166-175, for use in the treatment of a neurological, e.g., neurodegenerative, disorder, or a traumatic brain injury (TBI).
   209. The antibody of any one of embodiments 1-117, 125, or 126, the pharmaceutical composition of embodiment 183, or the AAV particle of any one of embodiments 166-175, for use in the treatment of a tauopathy.
   210. Use of an effective amount of the antibody of any one of embodiments 1-117, 125, or 126, the pharmaceutical composition of embodiment 183, or the AAV particle of any one of embodiments 166-175, in the manufacture of a medicament.
   211. Use of the antibody of any one of embodiments 1-117, 125, or 126, the pharmaceutical composition of embodiment 183, or the AAV particle of any one of embodiments 166-175, in the manufacture of a medicament for the treatment of a disease associated with tau expression, a neurological e.g., neurodegenerative, disorder, a tauopathy, mild cognitive impairment, or a traumatic brain injury (TBI).
   212. An AAV viral genome comprising a nucleotide sequence encoding an antibody that binds to tau, fragment or variant thereof, wherein the AAV viral genome comprises in 5′ to 3′ order:
(i) a 5′ adeno-associated (AAV) ITR, optionally wherein the 5′ AAV ITR comprises the nucleotide sequence of SEQ ID NO: 1035, or a nucleotide sequence at least 70%, 75%, 80%, 85%, 90%, 95% or 99% identical thereto;
(ii) an enhancer, optionally wherein the enhancer comprises the nucleotide sequence of 1050, or a nucleotide sequence at least 70%, 75%, 80%, 85%, 904, 95% or 99% identical thereto;
(iii) a promoter (e.g., a CBA promoter or a variant thereof), optionally wherein the promoter comprises the nucleotide sequence of SEQ ID NO: 1042, or a nucleotide sequence at least 70%, 75%, 80%, 85%, 90%, 95% or 99% identical thereto;
(iv) an intron, optionally wherein the intron comprises the nucleotide sequence of SEQ ID NO: 1067, or a nucleotide sequence at least 70%, 75%, 80%, 85%, 90%, 95% or 99% identical thereto;
(v) a nucleotide sequence encoding a first signal sequence, optionally wherein:
   (a) the first encoded signal sequence comprises the amino acid sequence of SEQ ID NO: 1; an amino acid sequence comprising one, two, three, but no more than four different amino acids relative to SEQ ID NO: 1; or an amino acid sequence comprising one, two, three, but no more than four modifications, e.g., substitutions, e.g., conservative substitutions, relative to SEQ ID NO: 1; and/or
   (b) the nucleotide sequence encoding the first signal sequence comprises the sequence of SEQ ID NO: 1083, or a nucleotide sequence at least 70%, 75%, 80%, 85%, 90%, 95% or 99% identical thereto;
(vi) a nucleotide sequence encoding a heavy chain variable region, wherein:
   (a) the encoded heavy chain variable region comprises the amino acid sequence of SEQ ID NO: 21, or an amino acid sequence at least 70%, 75%, 80%, 85%, 90%, 95% or 99% identical thereto; and/or
   (b) the nucleotide sequence encoding the heavy chain variable region comprises the nucleotide sequence of SEQ ID NO: 7, or a nucleotide sequence at least 70%, 75%, 80%, 85%, 90%, 95% or 99% identical thereto;
(v) a nucleotide sequence encoding a heavy chain constant region, optionally wherein:
   (a) the encoded heavy chain constant region comprises the amino acid sequence of SEQ ID NO: 16 or an amino acid sequence at least 70%, 75%, 80%, 85%, 90%, 95% or 99% identical thereto; and/or
   (b) the nucleotide sequence encoding the heavy chain constant region comprises the nucleotide sequence of SEQ ID NO: 805, or a nucleotide sequence at least 70%, 75%, 80%, 85%, 90%, 95% or 99% identical thereto;
(vi) a first linker, optionally wherein the first linker comprises the nucleotide sequence of SEQ ID NO: 1724; a nucleotide sequence comprising one, two, three but no more than four different nucleotides relative to the nucleotide sequence of SEQ ID NO: 1724; a nucleotide sequence comprising one, two, three but no more than four modifications, e.g., substitutions, relative to SEQ ID NO: 1724;
(vii) a second linker, optionally wherein the second linker comprises the nucleotide sequence of SEQ ID NO: 1726; a nucleotide sequence comprising one, two, three but no more than four different nucleotides relative to the nucleotide sequence of SEQ ID NO: 1726; a nucleotide sequence comprising one, two, three but no more than four modifications, e.g., substitutions, relative to SEQ ID NO: 1726;
(viii) a nucleotide sequence encoding a second signal sequence, optionally wherein:
   (a) the first encoded signal sequence comprises the amino acid sequence of SEQ ID NO: 2; an amino acid sequence comprising one, two, three, but no more than four different amino acids relative to SEQ ID NO: 2; or an amino acid sequence comprising one, two, three, but no more than four modifications, e.g., substitutions, e.g., conservative substitutions, relative to SEQ ID NO: 2; and/or
   (b) the nucleotide sequence encoding the second signal sequence comprises the sequence of SEQ ID NO: 1085, or a nucleotide sequence at least 70%, 75%, 80%, 85%, 90%, 95% or 99% identical thereto;
(ix) a nucleotide sequence encoding a light chain variable region, wherein:
   (a) the encoded light chain variable region comprises the amino acid sequence of SEQ ID NO: 93, or an amino acid sequence at least 70%, 75%, 80%, 85%, 90%, 95% or 99% identical thereto; and/or
   (b) the nucleotide sequence encoding the light chain variable region comprises the nucleotide sequence of SEQ ID NO: 11, or a nucleotide sequence at least 70%, 75%, 80%, 85%, 90%, 95% or 99% identical thereto;
(x) a nucleotide sequence encoding a light chain constant region, optionally wherein:
   (a) the encoded light chain constant region comprises the amino acid sequence of SEQ ID NO: 18, or an amino acid sequence at least 70%, 75%, 80%, 85%, 90%, 95% or 99% identical thereto; and/or
   (b) the nucleotide sequence encoding the light chain constant region comprises the nucleotide sequence of SEQ ID NO: 17, or a nucleotide sequence at least 70%, 75%, 80%, 85%, 90%, 95% or 99% identical thereto;
(xi) a polyA signal sequence, optionally wherein the polyA signal sequence comprises the nucleotide sequence of SEQ ID NO: 1134, or a nucleotide sequence at least 70%, 75%, 80%, 85%, 90%, 95% or 99% identical thereto; and
(xii) a 3′ AAV ITR, optionally wherein the 3′ AAV ITR comprises the nucleotide sequence of SEQ ID NO: 1037, or a nucleotide sequence at least 70%, 75%, 80%, 85%, 90%, 95% or 99% identical thereto.
   213. An AAV viral genome comprising the nucleotide sequence of SEQ ID NO: 15, or a nucleotide sequence at least 70%, 75%, 80%, 85%, 90%, 95% or 99% identical thereto.
   214. The viral genome of any one of embodiments 139-164 or 212, which comprises the nucleotide sequence of SEQ ID NO: 15, or a nucleotide sequence at least 70%, 75%, 80%, 85%, 90%, 95% or 99% identical thereto.

### BRIEF DESCRIPTION OF THE DRAWINGS

FIG. 1 is a graph depicting the stimulation index (ratio of the number of proliferating T cells of sample over blank) for each antibody following incubation with PBMC cells from 50 healthy donors representative for the global population based on HLA-DRB1 expression, as a measure of relative immunogenicity risk. The antibodies tested include, from left to right on the X axis, the Herceptin control, Ab2, Ab1, Ab3, Ab4, and Ab5. A simulation index greater than or equal to 2.0 is considered a positive response.

### DETAILED DESCRIPTION

### I. Compositions

In some embodiments, the present disclosure provides compositions that interact with human microtubule associated protein tau. Such compositions may be antibodies that bind tau protein epitopes, referred to herein as anti-tau antibodies. Dysfunction and/or aggregation of tau is found in a class of neurodegenerative diseases referred to as tauopathies. Tau hyperphosphorylation leads to aggregation and depressed tau-dependent microtubule assembly. In tauopathies, the tau aggregates form paired helical filaments (PHF) found in neurofibrillary tangles (NFTs). These aggregates lead to neuronal loss and cognitive decline. Anti-tau antibodies of the present disclosure may be useful for treating and/or diagnosing tauopathies, as well as other applications described herein.

### Antibodies

In some embodiments, compounds (e.g., anti-tau antibodies) and compositions of the present disclosure include antibodies or fragments thereof. In some embodiments, the antibody described herein bind tau. For example, the antibody binds to an epitope, e.g., a confirmation epitope, phosphorylated epitope, or a linear epitope, on tau, e.g., as described herein.

As used herein, the term “antibody” is referred to in the broadest sense and specifically covers various embodiments including, but not limited to monoclonal antibodies, polyclonal antibodies, multispecific antibodies (e.g. bispecific antibodies formed from at least two intact antibodies), single chain Fv (scFv) formats, and antibody fragments (such as Fab, F(ab′), F(ab′)₂, Fv, etc.), so long as they exhibit a desired functional or biological activity. Antibodies are primarily amino acid-based molecules but may also include one or more modifications (including, but not limited to the addition of sugar moieties, fluorescent moieties, chemical tags, etc.).

Antibodies (including antigen-binding fragments thereof) of the present disclosure may include, but are not limited to, polyclonal, monoclonal antibodies, multispecific antibodies, bispecific antibodies, trispecific antibodies, human antibodies, humanized antibodies, chimeric antibodies, single chain antibodies, diabodies, linear antibodies, Fab fragments, F(ab′) fragments, F(ab′)₂ fragments, Fv fragments, fragments produced by a Fab expression library, variable domains, anti-idiotypic (anti-Id) antibodies (including, e.g., anti-Id antibodies to antibodies of the invention), intracellularly made antibodies (i.e., intrabodies), codon-optimized antibodies, scFv fragments, tandem scFv antibodies, bispecific T-cell engagers, mAb2 antibodies, chimeric antigen receptors (CAR), tetravalent bispecific antibodies, biosynthetic antibodies, native antibodies, miniaturized antibodies, unibodies, maxibodies, and epitope-binding fragments of any of the above.

In some embodiments, the antibody comprises at least one immunoglobulin variable domain sequence. An antibody may include, for example, full-length, mature antibodies and antigen-binding fragments of an antibody. For example, an antibody can include a heavy (H) chain variable domain sequence (abbreviated herein as VH), and a light (L) chain variable domain sequence (abbreviated herein as VL). In another example, an antibody includes two heavy (H) chain variable domain sequences and two light (L) chain variable domain sequence, thereby forming two antigen binding sites, such as Fab, Fab′, F(ab′)₂, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., human IgG1, IgG2, IgG3, and IgG4, and murine IgG1, IgG2a, IgG2b, IgG2c, and IgG3) of antibodies. The antibodies of the present disclosure can be monoclonal or polyclonal. The antibody can also be a human, humanized, CDR-grafted, or in vitro generated antibody. The antibody can have a heavy chain constant region chosen from, e.g., IgG1, IgG2, IgG3, or IgG4. The antibody can also have a light chain chosen from, e.g., kappa or lambda.

In some embodiments, an antibody of the present disclosure comprises a functional fragment or variant thereof. Constant regions of the antibodies can be altered, e.g., mutated, to modify the properties of the antibody (e.g., to increase or decrease one or more of: Fc receptor binding, antibody glycosylation, the number of cysteine residues, effector cell function, or complement function).

As used herein, the term “antibody fragment” refers to a portion of an intact antibody or fusion-protein thereof, in some cases including at least one antigen binding region. Examples of antigen-binding fragments include: (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); and (viii) a single domain antibody. These antibody fragments are obtained using conventional techniques known to those with skill in the art, and the fragments are screened for utility in the same manner as are intact antibodies. An antibody fragment can also be incorporated into single domain antibodies, maxibodies, minibodies, nanobodies, intrabodies, diabodies, triabodies, tetrabodies, v-NAR and bis-scFv (see, for example, Hollinger and Hudson, Nature Biotechnology 23:1126-1136, 2005).” In some embodiments, papain digestion of antibodies produces two identical antigen-binding fragments, called “Fab” fragments, each with a single antigen-binding site. Also produced is a residual “Fc” fragment, whose name reflects its ability to crystallize readily. Pepsin treatment yields an F(ab′)₂ fragment that has two antigen-binding sites and is still capable of cross-linking antigen. Antibodies of the present disclosure may include one or more of these fragments and may, for example, be generated through enzymatic digestion of whole antibodies or through recombinant expression.

In some embodiments, the antibody can be single domain antibody. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to another aspect of the invention, a single domain antibody is a naturally occurring single domain antibody known as heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

“Native antibodies” are usually heterotetrameric glycoproteins of about 150,000 Daltons, composed of two identical light (L) chains and two identical heavy (H) chains. Genes encoding antibody heavy and light chains are known and segments making up each have been well characterized and described (Matsuda, F. et al., 1998. The Journal of Experimental Medicine. 188(11); 2151-62 and Li, A. et al., 2004. Blood. 103(12: 4602-9, the content of each of which are herein incorporated by reference in their entirety). Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain.

As used herein, the term “variable domain” refers to specific antibody domains found on both the antibody heavy and light chains that differ extensively in sequence among antibodies and are used in the binding and specificity of each particular antibody for its particular antigen. In some embodiments, the VH and VL regions of the antibody described herein can be subdivided into regions of hypervariability, termed complementarity determining regions (CDR), interspersed with regions that are more conserved, termed framework regions (FR).

As used herein, the term “hypervariable region” refers to a region within a variable domain that includes amino acid residues responsible for antigen binding. The amino acids present within the hypervariable regions determine the structure of the complementarity determining regions (CDRs) that become part of the antigen-binding site of the antibody.

As used herein, the term “CDR” refers to a region of an antibody that includes a structure that is complimentary to its target antigen or epitope. CDR regions generally confer antigen specificity and binding affinity. Other portions of the variable domain, not interacting with the antigen, are each referred to as a “framework region” (FR). The antigen-binding site (also known as the antigen combining site or paratope) includes the amino acid residues necessary to interact with a particular antigen. The exact residues making up the antigen-binding site may be determined by CDR analysis.

As used herein, the term “CDR analysis” refers to any process used to determine which antibody variable domain residues make up the CDRs. The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg). CDR analysis may be conducted by co-crystallography with bound antigen. In some embodiments, CDR analysis may include computational assessments based on comparisons with other antibodies (Strohl, W. R. Therapeutic Antibody Engineering. Woodhead Publishing, Philadelphia PA. 2012. Ch. 3, p47-54, the contents of which are herein incorporated by reference in their entirety). CDR analysis and/or the precise amino acid sequence boundaries may include the use of numbering schemes including, but not limited to, those taught by Kabat [Wu, T. T. et al., 1970, JEM, 132(2):211-50 and Johnson, G. et al., 2000, Nucleic Acids Res. 28(1): 214-8, the contents of each of which are herein incorporated by reference in their entirety], Chothia [Chothia and Lesk, J. Mol. Biol. 196, 901 (1987), Chothia et al., Nature 342, 877 (1989), and Al-Lazikani, B. et al., 1997, J. Mol. Biol. 273(4):927-48, the contents of each of which are herein incorporated by reference in their entirety], Lefranc (Lefranc, M. P. et al., 2005, Immunome Res. 1:3), and Honegger (Honegger, A. and Pluckthun, A. 2001. J. Mol. Biol. 309(3):657-70, the contents of which are herein incorporated by reference in their entirety). In some embodiments, the CDRs defined according the Chothia number scheme are also sometimes referred to as hypervariable loops.

For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3).

For example, under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3).

For example by combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL.

In general, the VH and VL domains have three CDRs each. VL CDRs are referred to herein as LC CDR1, LC CDR2 and LC CDR3, in order of occurrence when moving from N- to C-terminus along the variable domain polypeptide. VH CDRs are referred to herein as HC CDR1, HC CDR2, and HC CDR3, in order of occurrence when moving from N- to C-terminus along the variable domain polypeptide. Each of the CDRs have favored canonical structures with the exception of the HC CDR3, which includes amino acid sequences that may be highly variable in sequence and length between antibodies resulting in a variety of three-dimensional structures in antigen-binding domains (Nikoloudis, D. et al., 2014. PeerJ. 2:e456). In some cases, CDRH3s may be analyzed among a panel of related antibodies to assess antibody diversity. Various methods of determining CDR sequences are known in the art and may be applied to known antibody sequences (Strohl, W. R. Therapeutic Antibody Engineering. Woodhead Publishing, Philadelphia PA. 2012. Ch. 3, p47-54, the contents of which are herein incorporated by reference in their entirety).

In some embodiments, the antigen binding domain of the antibodies of the present disclosure is the part of the antibody that comprises determinants that form an interface that binds to the tau polypeptide or an epitope thereof. With respect to proteins (or protein mimetics), the antigen-binding site typically includes one or more loops (of at least four amino acids or amino acid mimics) that form an interface that binds to the tau polypeptide. Typically, the antigen-binding site of an antibody includes at least one or two CDRs and/or hypervariable loops, or more typically at least three, four, five or six CDRs and/or hypervariable loops.

In yet other embodiments, the antibody has a heavy chain constant region chosen from, e.g., the heavy chain constant regions of IgG1, IgG2, IgG3, IgG4, IgM, IgA1, IgA2, IgD, and IgE; particularly, chosen from, e.g., the human heavy chain constant regions of IgG1, IgG2, IgG3, and IgG4, or the murine heavy chain constant regions of IgG1, IgG2a, IgG2b, IgG2c, and IgG3. In another embodiment, the antibody has a light chain constant region chosen from, e.g., the (e.g., murine or human) light chain constant regions of kappa or lambda.

The constant region can be altered, e.g., mutated, to modify the properties of the antibody (e.g., to increase or decrease one or more of: Fc receptor binding, antibody glycosylation, the number of cysteine residues, effector cell function, and/or complement function). In some embodiments the antibody has: effector function; and can fix complement. In other embodiments the antibody does not recruit effector cells; or fix complement. In other embodiments, the antibody has reduced or no ability to bind an Fc receptor. For example, it is an isotype or subtype, fragment or other mutant, which does not support binding to an Fc receptor, e.g., it has a mutagenized or deleted Fc receptor binding region.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. Nos. 5,624,821 and 5,648,260, the contents of all of which are hereby incorporated by reference). Similar type of alterations could be described which if applied to the murine, or other species immunoglobulin would reduce or eliminate these functions.

As used herein, the term “Fv” refers to an antibody fragment that includes the minimum fragment on an antibody needed to form a complete antigen-binding site. These regions consist of a dimer of one heavy chain and one light chain variable domain in tight, non-covalent association. Fv fragments can be generated by proteolytic cleavage, but are largely unstable. Recombinant methods are known in the art for generating stable Fv fragments, typically through insertion of a flexible linker between the light chain variable domain and the heavy chain variable domain (to form a single chain Fv (scFv)] or through the introduction of a disulfide bridge between heavy and light chain variable domains (Strohl, W. R. Therapeutic Antibody Engineering. Woodhead Publishing, Philadelphia PA. 2012. Ch. 3, p46-47, the contents of which are herein incorporated by reference in their entirety).

Antibody “light chains” from any vertebrate species can be assigned to one of two clearly distinct types, called kappa and lambda based on amino acid sequences of their constant domains. Depending on the amino acid sequence of the constant domain of their heavy chains, antibodies can be assigned to different classes.

As used herein, the term “single chain Fv” or “scFv” refers to a fusion protein of VH and VL antibody domains, wherein these domains are linked together into a single polypeptide chain by a flexible peptide linker. In some embodiments, the Fv polypeptide linker enables the scFv to form the desired structure for antigen binding. In some embodiments, scFvs are utilized in conjunction with phage display, yeast display or other display methods where they may be expressed in association with a surface member (e.g. phage coat protein) and used in the identification of high affinity peptides for a given antigen. In some embodiments, antibodies of the present disclosure are prepared as scFvFc antibodies. The term “scFvFc” refers to an antibody format which includes the fusion of one or more scFv with an antibody Fc domain.

The term “chimeric antibody” refers to an antibody with portions derived from two or more sources. Chimeric antibodies may include portions derived from different species. For example, chimeric antibodies may include antibodies with mouse variable domains and human constant domains. Further examples of chimeric antibodies and methods for producing them include any of those described in Morrison, S. L., Transfectomas provide novel chimeric antibodies. Science. 1985 Sep. 20; 229(4719):1202-7; Gillies, S. D. et al., High-level expression of chimeric antibodies using adapted cDNA variable region cassettes. J Immunol Methods. 1989 Dec. 20; 125(1-2):191-202.; and U.S. Pat. Nos. 5,807,715; 4,816,567; and 4,816,397, the contents of each of which are incorporated herein by reference in their entirety.

The term “diabodies” refers to small antibody fragments with two antigen-binding sites, which fragments include a heavy chain variable domain V_{H} connected to a light chain variable domain V_{L} in the same polypeptide chain. By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natd. Acad. Sci. USA, 90:6444-6448 (1993), the contents of each of which are incorporated herein by reference in their entirety.

The term “intrabody” refers to a form of antibody that is not secreted from a cell in which it is produced, but instead targets one or more intracellular protein(s). Intrabodies may be used to affect a multitude of cellular processes including, but not limited to intracellular trafficking, transcription, translation, metabolic processes, proliferative signaling and cell division. In some embodiments, methods of the present invention may include intrabody-based therapies. In some such embodiments, variable domain sequences and/or CDR sequences disclosed herein may be incorporated into one or more constructs for intrabody-based therapy. In some cases, intrabodies of the invention may target one or more glycated intracellular proteins or may modulate the interaction between one or more glycated intracellular protein and an alternative protein.

The term “chimeric antigen receptor” or “CAR” as used herein, refers to artificial receptors that are engineered to be expressed on the surface of immune effector cells resulting in specific targeting of such immune effector cells to cells expressing entities that bind with high affinity to the artificial receptors. CARs may be designed to include one or more segments of an antibody, antibody variable domain and/or antibody CDR, such that when such CARs are expressed on immune effector cells, the immune effector cells bind and clear any cells that are recognized by the antibody portions of the CARs. In some cases, CARs are designed to specifically bind cancer cells, leading to immune-regulated clearance of the cancer cells.

The antibody of the invention can be a monoclonal antibody or a polyclonal antibody. The term “monoclonal antibody” as used herein refers to an antibody obtained from a population of substantially homogeneous cells (or clones), e.g., the individual antibodies making up the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen.

The modifier “monoclonal” indicates the character of the antibody as being obtained from a substantially homogeneous population of antibodies, and is not to be construed as requiring production of the antibody by any particular method.

In some embodiments, the antibody comprises an amino acid sequence of an antibody in which the variable region, or a portion thereof, e.g., the CDRs, are generated in a non-human organism, e.g., a rat or mouse. Antibodies comprising chimeric, CDR-grafted, and humanized antibodies are within the invention. Antibodies comprising the sequences of antibodies generated in a non-human organism, e.g., a rat or mouse, and then modified, e.g., in the variable framework or constant region, to decrease antigenicity in a human are within the invention.

The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies.

Antibodies of the present disclosure may be from any animal origin including mammals, birds, reptiles, and insects. Mammalian antibodies may be, for example, of human, murine (e.g., mouse or rat), donkey, sheep, rabbit, goat, guinea pig, camel, bovine, or horse origin.

In some embodiments, antibodies of the present disclosure may be antibody mimetics. The term “antibody mimetic” refers to any molecule which mimics the function or effect of an antibody and which binds specifically and with high affinity to their molecular targets. In some embodiments, antibody mimetics may be monobodies, designed to incorporate the fibronectin type III domain (Fn3) as a protein scaffold (U.S. Pat. Nos. 6,673,901; 6,348,584). In some embodiments, antibody mimetics may be those known in the art including, but are not limited to affibody molecules, affilins, affitins, anticalins, avimers, DARPins, Fynomers and Kunitz and domain peptides. In other embodiments, antibody mimetics may include one or more non-peptide region.

As used herein, the term “antibody variant” refers to a biomolecule resembling an antibody in structure, sequence and/or function, but including some differences in their amino acid sequence, composition or structure as compared to another antibody or a native antibody.

### Multispecific Antibodies

In some embodiments, the antibody is a multispecific antibody, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In some embodiments, the first and second epitopes are on the same antigen, e.g., the same protein (or subunit of a multimeric protein). In some embodiments, the first and second epitopes overlap. In some embodiments, the first and second epitopes do not overlap. In some embodiments, the first and second epitopes are on different antigens, e.g., the different proteins (or different subunits of a multimeric protein). In some embodiments, a multispecific antibody comprises a third, fourth or fifth immunoglobulin variable domain. In some embodiments, a multispecific antibody is a bispecific antibody, a trispecific antibody, or tetraspecific antibody. In some embodiments, the anti-tau antibody is a multispecific antibody.

In some embodiments, a multispecific antibody is a bispecific antibody. A bispecific antibody has specificity for no more than two antigens. A bispecific antibody is characterized by a first immunoglobulin variable domain sequence which has binding specificity for a first epitope and a second immunoglobulin variable domain sequence that has binding specificity for a second epitope. In some embodiments, the first and second epitopes are on the same antigen, e.g., the same protein (or subunit of a multimeric protein). In some embodiments, the first and second epitopes overlap. In some embodiments, the first and second epitopes do not overlap. In some embodiments, the first and second epitopes are on different antigens, e.g., the different proteins (or different subunits of a multimeric protein). In some embodiments, a bispecific antibody comprises a heavy chain variable domain sequence and a light chain variable domain sequence which have binding specificity for a first epitope and a heavy chain variable domain sequence and a light chain variable domain sequence which have binding specificity for a second epitope. In some embodiments, a bispecific antibody comprises a half antibody having binding specificity for a first epitope and a half antibody having binding specificity for a second epitope. In some embodiments, a bispecific antibody comprises a half antibody, or fragment thereof, having binding specificity for a first epitope and a half antibody, or fragment thereof, having binding specificity for a second epitope. In some embodiments, a bispecific antibody comprises a scFv, or fragment thereof, have binding specificity for a first epitope and a scFv, or fragment thereof, have binding specificity for a second epitope. In some embodiment, the anti-tau antibody is a bispecific antibody.

In some embodiments, the sequences of the antibody of the present disclosure can be generated from bispecific or heterodimeric antibody produced using protocols known in the art; including but not limited to, for example, the “knob in a hole” approach described in, e.g., U.S. Pat. No. 5,731,168; the electrostatic steering Fc pairing as described in, e.g., WO 09/089004, WO 06/106905 and WO 2010/129304; Strand Exchange Engineered Domains (SEED) heterodimer formation as described in, e.g., WO 07/110205; Fab arm exchange as described in, e.g., WO 08/119353, WO 2011/131746, and WO 2013/060867; double antibody conjugate, e.g., by antibody cross-linking to generate a bi-specific structure using a heterobifunctional reagent having an amine-reactive group and a sulfhydryl reactive group as described in, e.g., U.S. Pat. No. 4,433,059; bispecific antibody determinants generated by recombining half antibodies (heavy-light chain pairs or Fabs) from different antibodies through cycle of reduction and oxidation of disulfide bonds between the two heavy chains, as described in, e.g., U.S. Pat. No. 4,444,878; trifunctional antibodies, e.g., three Fab′ fragments cross-linked through sulfhdryl reactive groups, as described in, e.g., U.S. Pat. No. 5,273,743; biosynthetic binding proteins, e.g., pair of scFvs cross-linked through C-terminal tails preferably through disulfide or amine-reactive chemical cross-linking, as described in, e.g., U.S. Pat. No. 5,534,254; bifunctional antibodies, e.g., Fab fragments with different binding specificities dimerized through leucine zippers (e.g., c-fos and c-jun) that have replaced the constant domain, as described in, e.g., U.S. Pat. No. 5,582,996; bispecific and oligospecific mono- and oligovalent receptors, e.g., VH-CH1 regions of two antibodies (two Fab fragments) linked through a polypeptide spacer between the CH1 region of one antibody and the VH region of the other antibody typically with associated light chains, as described in, e.g., U.S. Pat. No. 5,591,828; bispecific DNA-antibody conjugates, e.g., crosslinking of antibodies or Fab fragments through a double stranded piece of DNA, as described in, e.g., U.S. Pat. No. 5,635,602; bispecific fusion proteins, e.g., an expression construct containing two scFvs with a hydrophilic helical peptide linker between them and a full constant region, as described in, e.g., U.S. Pat. No. 5,637,481; multivalent and multispecific binding proteins, e.g., dimer of polypeptides having first domain with binding region of Ig heavy chain variable region, and second domain with binding region of Ig light chain variable region, generally termed diabodies (higher order structures are also disclosed creating bispecific, trispecific, or tetraspecific molecules, as described in, e.g., U.S. Pat. No. 5,837,242; minibody constructs with linked VL and VH chains further connected with peptide spacers to an antibody hinge region and CH3 region, which can be dimerized to form bispecific/multivalent molecules, as described in, e.g., U.S. Pat. No. 5,837,821; VH and VL domains linked with a short peptide linker (e.g., 5 or 10 amino acids) or no linker at all in either orientation, which can form dimers to form bispecific diabodies; trimers and tetramers, as described in, e.g., U.S. Pat. No. 5,844,094; String of VH domains (or VL domains in family members) connected by peptide linkages with crosslinkable groups at the C-terminus further associated with VL domains to form a series of FVs (or scFvs), as described in, e.g., U.S. Pat. No. 5,864,019; and single chain binding polypeptides with both a VH and a VL domain linked through a peptide linker are combined into multivalent structures through non-covalent or chemical crosslinking to form, e.g., homobivalent, heterobivalent, trivalent, and tetravalent structures using both scFV or diabody type format, as described in, e.g., U.S. Pat. No. 5,869,620.

### Intrabody

In some embodiments, payloads may encode intrabodies. Intrabodies are a form of antibody that is not secreted from a cell in which it is produced, but instead targets one or more intracellular proteins. Intrabodies are expressed and function intracellularly, and may be used to affect a multitude of cellular processes including, but not limited to intracellular trafficking, transcription, translation, metabolic processes, proliferative signaling and cell division. In some embodiments, methods described herein include intrabody-based therapies. In some such embodiments, variable domain sequences and/or CDR sequences disclosed herein are incorporated into one or more constructs for intrabody-based therapy. For example, intrabodies may target one or more glycated intracellular proteins or may modulate the interaction between one or more glycated intracellular proteins and an alternative protein.

More than two decades ago, intracellular antibodies against intracellular targets were first described (Biocca, Neuberger and Cattaneo EMBO J. 9: 101-108, 1990). The intracellular expression of intrabodies in different compartments of mammalian cells allows blocking or modulation of the function of endogenous molecules (Biocca, et al., EMBO J. 9: 101-108, 1990; Colby et al., Proc. Natd. Acad. Sci. U.S.A. 101: 17616-21, 2004). Intrabodies can alter protein folding, protein-protein, protein-DNA, protein-RNA interactions and protein modification. They can induce a phenotypic knockout and work as neutralizing agents by direct binding to the target antigen, by diverting its intracellular trafficking or by inhibiting its association with binding partners. They have been largely employed as research tools and are emerging as therapeutic molecules for the treatment of human diseases such as viral pathologies, cancer and misfolding diseases. The fast-growing bio-market of recombinant antibodies provides intrabodies with enhanced binding specificity, stability, and solubility, together with lower immunogenicity, for their use in therapy (Biocca, abstract in Antibody Expression and Production Cell Engineering Volume 7, 2011, pp. 179-195).

In some embodiments, intrabodies have advantages over interfering RNA (iRNA); for example, iRNA has been shown to exert multiple non-specific effects, whereas intrabodies have been shown to have high specificity and affinity to target antigens. Furthermore, as proteins, intrabodies possess a much longer active half-life than iRNA. Thus, when the active half-life of the intracellular target molecule is long, gene silencing through iRNA may be slow to yield an effect, whereas the effects of intrabody expression can be almost instantaneous. Lastly, it is possible to design intrabodies to block certain binding interactions of a particular target molecule, while sparing others.

Intrabodies are often single chain variable fragments (scFvs) expressed from a recombinant nucleic acid molecule and engineered to be retained intracellularly (e.g., retained in the cytoplasm, endoplasmic reticulum, or periplasm). Intrabodies may be used, for example, to ablate the function of a protein to which the intrabody binds. The expression of intrabodies may also be regulated through the use of inducible promoters in the nucleic acid expression vector comprising the intrabody. Intrabodies may be produced for use in the viral genomes using methods known in the art, such as those disclosed and reviewed in: (Marasco et al., 1993 Proc. Natd. Acad. Sci. USA, 90: 7889-7893; Chen et al., 1994, Hum. Gene Ther. 5:595-601; Chen et al., 1994, Proc. Natl. Acad. Sci. USA, 91: 5932-5936; Maciejewski et al., 1995, Nature Med., 1: 667-673; Marasco, 1995, Immunotech, 1: 1-19; Mhashilkar, et al., 1995, EMBO J. 14: 1542-51; Chen et al., 1996, Hum. Gene Therap., 7: 1515-1525; Marasco, Gene Ther. 4:11-15, 1997; Rondon and Marasco, 1997, Annu. Rev. Microbiol. 51:257-283; Cohen, et al., 1998, Oncogene 17:2445-56; Proba et al., 1998, J. Mol. Biol. 275:245-253; Cohen et al., 1998, Oncogene 17:2445-2456; Hassanzadeh, et al., 1998, FEBS Lett. 437:81-6; Richardson et al., 1998, Gene Ther. 5:635-44; Ohage and Steipe, 1999, J. Mol. Biol. 291:1119-1128; Ohage et al., 1999, J. Mol. Biol. 291:1129-1134; Wirtz and Steipe, 1999, Protein Sci. 8:2245-2250; Zhu et al., 1999, J. Immunol. Methods 231:207-222; Arafat et al., 2000, Cancer Gene Ther. 7:1250-6; der Maur et al., 2002, J. Biol. Chem. 277:45075-85; Mhashilkar et al., 2002, Gene Ther. 9:307-19; and Wheeler et al., 2003, FASEB J. 17: 1733-5; and references cited therein). In particular, a CCR5 intrabody has been produced by Steinberger et al., 2000, Proc. Natl. Acad. Sci. USA 97:805-810). See generally Marasco, WA, 1998, “Intrabodies: Basic Research and Clinical Gene Therapy Applications” Springer: New York; and for a review of scFvs, see Pluckthun in “The Pharmacology of Monoclonal Antibodies,” 1994, vol. 113, Rosenburg and Moore eds. Springer-Verlag, New York, pp. 269-315.

Sequences from donor antibodies may be used to develop intrabodies. Intrabodies are often recombinantly expressed as single domain fragments such as isolated VH and VL domains or as a single chain variable fragment (scFv) antibody within the cell. For example, intrabodies are often expressed as a single polypeptide to form a single chain antibody comprising the variable domains of the heavy and light chains joined by a flexible linker polypeptide. Intrabodies typically lack disulfide bonds and are capable of modulating the expression or activity of target genes through their specific binding activity. Single chain antibodies can also be expressed as a single chain variable region fragment joined to the light chain constant region.

As is known in the art, an intrabody can be engineered into recombinant polynucleotide vectors to encode sub-cellular trafficking signals at its N or C terminus to allow expression at high concentrations in the sub-cellular compartments where a target protein is located. For example, intrabodies targeted to the endoplasmic reticulum (ER) are engineered to incorporate a leader peptide and, optionally, a C-terminal ER retention signal, such as the KDEL amino acid motif (SEQ ID NO: 4545). Intrabodies intended to exert activity in the nucleus are engineered to include a nuclear localization signal. Lipid moieties are joined to intrabodies in order to tether the intrabody to the cytosolic side of the plasma membrane. Intrabodies can also be targeted to exert function in the cytosol. For example, cytosolic intrabodies are used to sequester factors within the cytosol, thereby preventing them from being transported to their natural cellular destination.

There are certain technical challenges with intrabody expression. In particular, protein conformational folding and structural stability of the newly synthesized intrabody within the cell is affected by reducing conditions of the intracellular environment.

Intrabodies may be promising therapeutic agents for the treatment of misfolding diseases, including tauopathies, prion diseases, Alzheimer's, Parkinson's, and Huntington's, because of their virtually infinite ability to specifically recognize the different conformations of a protein, including pathological isoforms, and because they can be targeted to the potential sites of aggregation (both intra- and extracellular sites). These molecules can work as neutralizing agents against amyloidogenic proteins by preventing their aggregation, and/or as molecular shunters of intracellular traffic by rerouting the protein from its potential aggregation site (Cardinale, and Biocca, Curr. Mol. Med. 2008, 8:2-11).

### Antibody Development

Antibodies according to the present disclosure may be developed using methods standard in the art. Two primary antibody preparation technologies are immunization and antibody display technology. In either case, desired antibodies are identified from a larger pool of candidates based on affinity for a specific target or epitope. An immune response is characterized by the reaction of the cells, tissues and/or organs of an organism to the presence of a foreign entity. Such an immune response typically leads to the production by the organism of one or more antibodies against the foreign entity, e.g., antigen or a portion of the antigen.

### Antigens

Antibodies may be developed (e.g., through immunization) or selected (e.g., from pool of candidates), for example, using any naturally occurring or synthetic antigen. As used herein, an “antigen” is an entity which induces or evokes an immune response in an organism and may also refer to an antibody binding partner. An immune response is characterized by the reaction of the cells, tissues and/or organs of an organism to the presence of a foreign entity. Such an immune response typically leads to the production by the organism of one or more antibodies against the foreign entity. In some embodiments, antigens include tau proteins.

As used herein, the term “tau protein” refers to proteins or protein complexes that include microtubule-associated protein tau or peptide fragments thereof. Tau proteins may include enriched paired helical filament tau protein (ePHF), also referred to as “sarkosyl insoluble tau,” or fragments thereof. Tau proteins may include one or more phosphorylated residues. Such phosphorylated residues may correspond to tau proteins associated with disease (also referred to herein as “pathological tau”).

### Immunization

In some embodiments, antibodies may be prepared by immunizing a host with an antigen of interest. Host animals (e.g., mice, rabbits, goats, or llamas) may be immunized with an antigenic protein to elicit lymphocytes that specifically bind to the antigen. Lymphocytes may be collected and fused with immortalized cell lines to generate hybridomas which can be cultured in a suitable culture medium to promote growth (e.g., see Kohler, G. et al., Continuous cultures of fused cells secreting antibody of predefined specificity. Nature. 1975 Aug. 7; 256(5517):495-7, the contents of which are herein incorporated by reference in their entirety). Alternatively, lymphocytes may be immunized in vitro.

Lymphocytes may be fused with immortalized cell lines using suitable fusing agents (e.g., polyethylene glycol) to form a hybridoma cell (e.g., see Goding, J. W., Monoclonal Antibodies: Principles and Practice. Academic Press. 1986; 59-1031, the contents of which are herein incorporated by reference in their entirety). Immortalized cell lines may be transformed mammalian cells, particularly myeloma cells of rodent, rabbit, bovine, or human origin. In some embodiments, rat or mouse myeloma cell lines are employed. Hybridoma cells may be cultured in suitable culture media, typically including one or more substances that inhibit the growth or survival of unfused cells. For example, parental cells lacking the enzyme hypoxanthine guanine phosphoribosyl transferase (HGPRT or HPRT) may be used and culture media for resulting hybridoma cells may be supplemented with hypoxanthine, aminopterin, and thymidine (“HAT medium”) to prevent growth of HGPRT-deficient (unfused) cells.

Desirable properties for immortalized cell lines may include, but are not limited to, efficient fusing, supportive of high level antibody expression by selected antibody-producing cells, and sensitivity to unfused cell-inhibitory media (e.g., HAT media). In some embodiments, immortalized cell lines are murine myeloma lines. Such cell lines may be obtained, for example, from the Salk Institute Cell Distribution Center (San Diego, CA) or the American Type Culture Collection, (Manassas, VA). Human myeloma and mouse-human heteromyeloma cell lines may also be used for the production of human monoclonal antibodies (e.g., see Kozbor, D. et al., A human hybrid myeloma for production of human monoclonal antibodies. J Immunol. 1984 December; 133(6):3001-5 and Brodeur, B. et al., Monoclonal Antibody Production Techniques and Applications. Marcel Dekker, Inc., New York. 1987; 33:51-63, the contents of each of which are herein incorporated by reference in their entireties).

Hybridoma cell culture media may be assayed for the presence of monoclonal antibodies with desired binding specificity. Assays may include, but are not limited to, immunoprecipitation assay, in vitro binding assay, radioimmunoassay (RIA), surface plasmon resonance (SPR) assay, and/or enzyme-linked immunosorbent assay (ELISA). In some embodiments, binding specificity of monoclonal antibodies may be determined by Scatchard analysis (Munson, P. J. et al., Ligand: a versatile computerized approach for characterization of ligand-binding systems. Anal Biochem. 1980 Sep. 1; 107(1):220-39, the contents of which are herein incorporated by reference in their entirety).

Antibodies produced by cultured hybridomas may be analyzed to determine binding specificity for target antigens. Once antibodies with desirable characteristics are identified, corresponding hybridomas may be subcloned through limiting dilution procedures and grown by standard methods. Antibodies produced by hybridomas may be isolated and purified using standard immunoglobulin purification procedures such as, for example, protein A-Sepharose, hydroxyapatite chromatography, gel electrophoresis, dialysis, or affinity chromatography. Alternatively, hybridoma cells may be grown in vivo as ascites in a mammal. In some embodiments, antibodies may be isolated directly from serum of immunized hosts.

In some embodiments, recombinant versions of antibodies generated through immunization may be prepared. Such antibodies may be prepared using genomic antibody sequences from selected hybridomas. Hybridoma genomic antibody sequences may be obtained by extracting RNA molecules from antibody-producing hybridoma cells and producing cDNA by reverse transcriptase polymerase chain reaction (PCR). PCR may be used to amplify cDNA using primers specific for antibody heavy and light chains. PCR products may then be subcloned into plasmids for sequence analysis. Antibodies may be produced by insertion of resulting antibody sequences into expression vectors. Some recombinant antibodies may be prepared using synthetic nucleic acid constructs that encode amino acid sequences corresponding to amino acid sequences obtained from isolated hybridoma antibodies.

### Antibody Display

In some embodiments, antibodies may be developed using antibody display technologies. “Display technology” refers to systems and methods for expressing amino acid-based candidate compounds in a format where they are linked with nucleic acids encoding them and are accessible to a target or ligand. Candidate compounds are expressed at the surface of a host capsid or cell in most systems, however, some host-free systems (e.g., ribosomal display) exist. Display technologies may be used to generate display “libraries,” which include sets of candidate compound library members. Display libraries with antibodies (or variants or fragments thereof) as library members are referred to herein as “antibody display libraries.” Antibodies may be designed, selected, or optimized by screening target antigens using antibody display libraries. Antibody display libraries may include millions to billions of members, each expressing unique antibody domains. Antibody fragments displayed may be scFv antibody fragments, which are fusion proteins of VH and V_{L} antibody domains joined by a flexible linker. Display libraries may include antibody fragments with differing levels of diversity between variable domain framework regions and CDRs. Display library antibody fragment CDRs may include unique variable loop lengths and/or sequences. Antibody variable domains or CDRs obtained from display library selection may be directly incorporated into antibody sequences for recombinant antibody production or mutated and utilized for further optimization through in vitro affinity maturation.

Antibody display libraries may include antibody phage display libraries. Antibody phage display libraries utilize phage virus particles as hosts with millions to billions of members, each expressing unique antibody domains. Such libraries may provide richly diverse sources that may be used to select potentially hundreds of antibody fragments with diverse levels of affinity for one or more antigens of interest (McCafferty, et al., 1990. Nature. 348:552-4; Edwards, B. M. et al., 2003. JMB. 334: 103-18; Schofield, D. et al., 2007. Genome Biol. 8, R254 and Pershad, K. et al., 2010. Protein Engineering Design and Selection. 23:279-88; the contents of each of which are herein incorporated by reference in their entirety). Antibody fragments displayed may be scFv antibody fragments. Phage display library members may be expressed as fusion proteins, linked to viral coat proteins (e.g. the N-terminus of the viral pIII coat protein). V_{L} chains may be expressed separately for assembly with VH chains in the periplasm prior to complex incorporation into viral coats. Precipitated library members may be sequenced from the bound phage to obtain cDNA encoding desired antibody domains.

In some embodiments, antibody display libraries may be generated using yeast surface display technology. Antibody yeast display libraries are made up of yeast cells with surface displayed antibodies or antibody fragments. Antibody yeast display libraries may include antibody variable domains expressed on the surface of Saccharomyces cerevisiae cells. Yeast display libraries may be developed by displaying antibody fragments of interest as fusion proteins with yeast surface proteins (e.g. Aga2p protein). Yeast cells displaying antibodies or antibody fragments with affinity for a specific target may be isolated according to standard methods. Such methods may include, but are not limited to, magnetic separation and flow cytometry.

### Recombinant Synthesis

Antibodies of the present disclosure may be prepared using recombinant DNA technology and related processes. Constructs (e.g., DNA expression plasmids) encoding antibodies may be prepared and used to synthesize full antibodies or portions thereof. In some embodiments, DNA sequences encoding antibody variable domains of the present disclosure may be inserted into expression vectors (e.g., mammalian expression vectors) encoding other antibody domains and used to prepare antibodies with the inserted variable domains. DNA sequences encoding antibody variable domains may be inserted downstream of upstream expression vector regions with promoter/enhancer elements and/or encoding immunoglobulin signal sequences. DNA sequences encoding antibody variable domains may be inserted upstream of downstream expression vector regions encoding immunoglobulin constant domains. Encoded constant domains may be from any class (e.g., IgG, IgA, IgD, IgE, and IgM) or species (e.g., human, mouse, rabbit, rat, and non-human primate). In some embodiments, encoded constant domains encode human IgG (e.g., IgG1, IgG2, IgG3, or IgG4) constant domains. In some embodiments, encoded constant domains encode mouse IgG (e.g., IgG1, IgG2a, IgG2b, IgG2c, or IgG3) constant domains.

Expression vectors encoding antibodies of the present disclosure may be used to transfect cells for antibody production. Such cells may be mammalian cells. Cell lines with stable transfection of antibody expression vectors may be prepared and used to establish stable cell lines. Cell lines producing antibodies may be expanded for expression of antibodies which may be isolated or purified from cell culture media.

### Antibody Characterization

In some embodiments, antibodies of the present disclosure may be identified, selected, or excluded based on different characteristics. Such characteristics may include, but are not limited to, physical and functional characteristics. Physical characteristics may include features of antibody structures [e.g., amino acid sequence or residues; secondary, tertiary, or quaternary protein structure; post-translational modifications (e.g., glycosylations); chemical bonds, and stability]. Functional characteristics may include, but are not limited to, antibody affinity (i.e., for specific epitopes and/or antigens) and antibody activity (e.g., antibody ability to activate or inhibit a target, process, or pathway).

### Antibody Binding and Affinity

In some embodiments, antibodies of the present disclosure may be identified, selected, or excluded based on binding and/or level of affinity for specific epitopes and/or antigens. Antibody binding and/or affinity level may be assessed with different antigen formats. In some embodiments, antibody affinity for different antigen formats may be tested in vitro (e.g., by ELISA). Anti-tau antibody in vitro testing may be carried out using brain samples or fractions. Such samples or fractions may be obtained from subjects with AD (e.g., human AD patients). In some embodiments, brain samples or fractions may be obtained from non-human subjects. Such non-human subjects may include non-human animals used in AD disease model studies (e.g., mice, rats, and primates). In some embodiments, brain samples or fractions used for antibody affinity testing may be derived from TG4510/P301S mouse strains. Antibody affinity may be compared against control samples lacking the particular antigen for which affinity is being analyzed. In some embodiments, control samples used for anti-tau antibody testing may include brain samples or fractions from non-diseased human subjects. In some embodiments, brain samples or fractions from wild type and/or Tau knockout mouse strains may be used as control samples.

In vitro affinity testing may be carried out (e.g., by ELISA) using recombinant or isolated protein antigens. For example, recombinant or isolated ePHF may be used for anti-tau antibody affinity testing. In some embodiments, anti-tau antibodies of the present disclosure may exhibit a half maximal effective concentration (EC50) of from about 0.01 nM to about 100 nM for binding to ePHF when assessed by ELISA. In some embodiments, the exhibited EC50 may be less than about 50 nM, less than about 20 nM, less than about 10 nM, or less than about 1 nM. In some embodiments, anti-tau antibodies of the present disclosure may exhibit an EC50 of from about 0.01 nM to about 100 nM for binding to any of the antigens listed in Table 8, or an epitope that includes or is included within any of the antigens (including, but not limited to conformational epitopes), when assessed by ELISA. In some embodiments, the exhibited EC50 may be less than about 50 nM, less than about 20 nM, less than about 10 nM, or less than about 1 nM.

In some embodiments, anti-tau antibodies of the present disclosure bind to pathological tau, but do not bind to non-pathological tau. Such antibodies may be referred to herein as being “selective” for pathological forms of tau. In some embodiments, anti-tau antibodies of the present disclosure bind to tau tangles.

In some embodiments, antibody affinity analysis may be used to identify, select, or exclude polyspecific antibodies. As used herein, the term “polyspecific antibody” refers to an antibody with affinity for more than one epitope or antigen. In some embodiments, polyspecific antibodies may be identified, selected, or excluded based on relative affinity for each epitope or antigen recognized. For example, a polyspecific antibody may be selected for use or further development based on higher affinity for one epitope or antigen over a second epitope or antigen for which the polyspecific antibody demonstrates affinity.

In some embodiments, anti-tau antibodies may be tested for competition with other anti-tau antibodies. Such testing may be carried out to provide information on the specific epitope recognized by an antibody and may yield information related to level of epitope affinity in comparison to the competing antibody. In some embodiments, anti-tau antibodies used in antibody binding and/or affinity analysis may include anti-tau antibody PT3, as described in U.S. Pat. No. 9,371,376; anti-tau antibody C10.2, as described in U.S. Pat. No. 10,196,439 (referred to as antibody “C10-2,” therein); anti-tau antibody IPN002, as described in U.S. Pat. No. 10,040,847; anti-tau antibody AT8 (ThermoFisher, Waltham, MA); anti-tau antibody AT100 (ThermoFisher, Waltham, MA); anti-tau antibody AT120 as described in U.S. Pat. No. 5,843,779; or anti-tau antibody PT76, as described in Vandermeeren, M. et al., J Alzheimers Dis. 2018; 65(1):265-281.

### Antibody Activity

In some embodiments, antibodies of the present disclosure may be identified, selected, or excluded based on their ability to promote or reduce a certain activity. Antibody activity may be assessed using analytical assays. Such assays may be selected or designed to detect, screen, measure, and/or rank antibodies based on such antibody activity.

Anti-tau antibodies may be characterized by ability to inhibit tau aggregation. Inhibition may be based on physical disruption of tau aggregation or may be based on anti-tau antibody-dependent depletion (immunodepletion) of tau protein. Characterization based on tau aggregation inhibition may be assessed using one or more assays of tau aggregation. In some embodiments, anti-tau antibodies may be characterized by tau seeding assay. Tau seeding assays typically involve in vitro initiation of tau aggregation and assessment of aggregation inhibition by candidate compounds being tested. Tau seeding assays may be carried out using tau aggregation biosensor cells. Tau aggregation biosensor cells yield a detectable signal (e.g., a fluorescent signal) in response to tau aggregation. Tau aggregation biosensor cells may be cultured with recombinant or isolated tau or with samples from high tau brain tissues or fluids (to promote tau aggregation) and treated with or without candidate compounds to assess tau aggregation inhibition. In some embodiments, anti-tau antibodies may be used to deplete tau from media prior to incubation with biosensor cells. Aggregation levels with depleted media may be compared to aggregation levels with non-depleted media to assess anti-tau antibody inhibitory function. Tau aggregation biosensor cells may include, but are not limited to, tau RD Biosensor cells. In some embodiments, neurons expressing human tau may be used.

In some embodiments, anti-tau antibodies of the present disclosure may inhibit tau aggregation with a half maximal inhibitory concentration (IC50) of from about 1 nM to about 30 nM as determined by immunodepletion assay (e.g., using tau RD Biosensor cells).

### Antibody Modification

Antibodies may be modified to obtain variants with one or more altered properties. Such properties may include or relate to antibody structure, function, affinity, specificity, protein folding, stability, manufacturing, expression, and/or immunogenicity (i.e., immune reactions in subjects being treated with such antibodies). In some embodiments, antibody fragments or variants may be used to modify another antibody or may be incorporated into a synthetic antibody.

Antibody modification may include amino acid sequence modifications. Such modifications may include, but are not limited to, amino acid deletions, additions, and/or substitutions. Modifications may be informed by amino acid sequence analysis. Such analysis may include alignment of amino acid sequences between different antibodies or antibody variants. Two or more antibodies may be compared to identify residues or regions suitable for modification. Compared antibodies may include those binding to the same epitope. Compared antibodies may bind to different epitopes (separate or overlapping) of the same protein or target (e.g., to identify residues or regions conferring specificity to specific epitopes). Comparisons may include light and/or heavy chain sequence variation analysis, CDR sequence variation analysis, germline sequence analysis, and/or framework sequence analysis. Information obtained from such analysis may be used to identify amino acid residues, segments of amino acids, amino acid side chains, CDR lengths, and/or other features or properties that are conserved or variable among antibodies binding to the same or different epitopes.

In some embodiments, modified versions of anti-tau antibodies described above may be prepared by adding, deleting, or substituting one or more CDR amino acid residues.

In some embodiments, anti-tau antibodies may be modified by amino acid sequence alignment of antibodies binding to similar targets and preparing modified antibodies with one or more amino acid deletions, substitutions, or insertions based on analysis of the aligned sequences.

The present disclosure includes amino acid consensus sequences for CDR region sequences, showing specific amino acids that may be modified or amino acid residue positions that may be more generally substituted (shown using variable “X”) in antibody amino acid sequences, e.g. as described in Table 1.

TABLE 1
Consensus CDRs for exemplary anti-tau antibodies V0022, V0023 and V0024

| SEQ ID NO | Description | Sequence |
|---|---|---|
| SEQ ID NO: 1180 | HCDR1 | GX₁TFTX₂X₃, wherein |
| (Chothia) | | X₁ is F or Y; |
| | | X₂ is R or I; and |
| | | X3 is Y or F |
| SEQ ID NO: 341 | HCDR2 | NPNNGG |
| (Chothia) | | |
| SEQ ID NO: 410 | HCDR3 | GTGTGAMDY |
| (Chothia) | | |
| SEQ ID NO: 1181 | LCDR1 | RSSQSLVHX₁NGX₂TX₃LY, wherein |
| (Chothia) | | X₁ is N or S; |
| | | X₂ is I or N; and |
| | | X₃ is Y or H |
| SEQ ID NO: 1182 | LCDR2 | RVSX₁RFS, wherein |
| (Chothia) | | X₁ is N or S |
| SEQ ID NO: 571 | LCDR3 | FQGTHVPRT |
| (Chothia) | | |
| (Kabat) | HCDR1 | X₁X₂WMH, wherein |
| | | X₁ is R or I; and |
| | | X₂ is Y or F |
| SEQ ID NO: 1184 | HCDR2 | X₁INPNNGGX₂DX₃NEX₄FKX₅, wherein |
| (Kabat) | | X₁ is N or K; |
| | | X₂ is T or G; |
| | | X₃ is F, Y, or N; |
| | | X₄ is K or R; and |
| | | X₅ is N or S; |
| SEQ ID NO: 410 | HCDR3 | GTGTGAMDY |
| (Kabat) | | |
| SEQ ID NO: 1185 | LCDR1 | RSSQSLVHX₁NGX₂TX₃LY, wherein |
| (Kabat) | | X₁ is N or S; |
| | | X₂ is I or N; and |
| | | X₃ is Y or H |
| SEQ ID NO: 1182 | LCDR2 | RVSX₁RFS, wherein |
| (Kabat) | | X₁ is N or S |
| SEQ ID NO: 571 | LCDR3 | FQGTHVPRT |
| (Kabat) | | |
| SEQ ID NO: 1186 | HCDR1 | GX₁TFTX₂X₃W, wherein |
| (IMGT) | | X₁ is F or Y; |
| | | X₂ is R or I; and |
| | | X3 is Y or F |
| SEQ ID NO: 1187 | HCDR2 | INPNNGG X₁, wherein |
| (IMGT) | | X₁ is T or G |
| SEQ ID NO: 1167 | HCDR3 | ARGTGTGAMDY |
| (IMGT) | | |
| SEQ ID NO: 1188 | LCDR1 | QSLVHX₁NGX₂TX₃, wherein |
| (IMGT) | | X₁ is N or S; |
| | | X₂ is I or N; and |
| | | X₃ is Y or H |
| (IMGT) | LCDR2 | RVS |
| SEQ ID NO: 571 | LCDR3 | FQGTHVPRT |
| (IMGT) | | |

### Functional Modifications

In some embodiments, antibodies of the present disclosure may be modified to optimize one or more functional properties (e.g., antibody affinity or activity). Non-limiting examples of antibody functional properties include epitope or antigen affinity, ability to mobilize or immobilize targets, and ability to activate or inhibit a target, process, or pathway. In some embodiments, functional properties include or relate to ability to modulate protein-protein interactions, protein aggregation, enzyme activity, receptor-ligand interactions, cellular signaling pathways, proteolytic cascades, and/or biological or physiological responses.

Antibody modifications may optimize antibodies by modulating epitope affinity. Such modifications may be carried out by affinity maturation. Affinity maturation technology is used to identify sequences encoding CDRs with highest affinity for target antigens. In some embodiments, antibody display technologies (e.g., phage or yeast) may be used. Such methods may include mutating nucleotide sequences encoding parental antibodies being optimized. Nucleotide sequences may be mutated randomly as a whole or to vary expression at specific amino acid residues to create millions to billions of variants. Sites or residues may be selected for mutation based on sequences or amino acid frequencies observed in natural human antibody repertoires. Variants may be subjected to repeated rounds of affinity screening (e.g., using display library screening technologies, surface plasmon resonance technologies, fluorescence-associated cell sorting (FACS) analysis, enzyme-linked immunosorbent assay (ELISA), etc.) for target antigen binding. Repeated rounds of selection, mutation, and expression may be carried out to identify antibody fragment sequences with highest affinity for target antigens. Such sequences may be directly incorporated into antibody sequences for production. In some cases, the goal of affinity maturation is to increase antibody affinity by at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 100 fold, at least 500-fold, at least 1,000-fold, or more than 1,000-fold as compared to the affinity of an original or starting antibody. In cases where affinity is less than desired, the process may be repeated.

In some embodiments, antibody affinity may be assessed with different antigen formats. In some embodiments, antibody affinity for different antigen formats may be tested in vitro (e.g., by ELISA). In vitro testing may be carried out using brain samples or fractions. Such samples or fractions may be obtained from subjects with AD (e.g., human AD patients). In some embodiments, brain samples or fractions may be obtained from non-human subjects. Such non-human subjects may include non-human animals used in AD disease model studies (e.g., mice, rats, and primates). In some embodiments, brain samples or fractions used for antibody affinity testing may be derived from TG4510/P301S mouse strains. Antibody affinity may be compared against control samples lacking the particular antigen for which affinity is being analyzed. In some embodiments, control samples may include brain samples or fraction from non-diseased human subjects. In some embodiments, brain samples or fractions from wild type and/or Tau knockout mouse strains may be used as control samples. In vitro affinity testing may be carried out (e.g., by ELISA) using recombinant or isolated protein antigens. In some embodiments, recombinant or isolated ePHF is used for antibody affinity testing. In some embodiments, antigens listed in Table 8 may be used.

In some embodiments, antibody affinity analysis may be used to modulate antibody polyspecificity (e.g., to reduce or enhance antibody polyspecificity). Such modulation may include modulating relative affinity for two or more epitopes or antigens. For example, antibodies may be optimized for higher affinity for one epitope or antigen over a second epitope or antigen.

Antibodies may be modified to optimize antibody functional properties. Such functional properties may be assessed or engineered based on analytical assay results relating to one or more antibody functional properties. Assays may be used to screen multiple antibodies to identify or rank antibodies based on functional criteria. Anti-tau antibodies may be modified to optimize tau aggregation inhibition. Such inhibition may be based on physical disruption of tau aggregation or may be based on the ability of anti-tau antibodies to deplete tau protein from assay samples. Optimization based on tau aggregation inhibition may be assessed using one or more assays of tau aggregation (e.g., by tau seeding assay).

### Production Modifications

In some embodiments, modifications may be made to optimize antibody production. Such modifications may include or relate to one or more of protein folding, stability, expression, and/or immunogenicity. Modifications may be carried out to address one or more antibody features negatively impacting production. Such features may include, but are not limited to, unpaired cysteines or irregular disulfides; glycosylation sites (e.g., N-linked NXSiT sites); acid cleavage sites, amino acid oxidation sites, conformity with mouse germline sequences; asparagine deamidation sites; aspartate isomerization sites; N-terminal pyroglutamate formation sites; and aggregation-prone amino acid sequence regions (e.g., within CDR sequences).

In some embodiments, antibodies of the present disclosure may be prepared using recombinant DNA technology (e.g., see U.S. Pat. No. 4,816,567, which is hereby incorporated by reference in its entirety). Antibody-encoding DNA may be isolated and sequenced using conventional procedures (e.g., by using oligonucleotide probes that are capable of binding specifically to genes encoding the heavy and light chains of murine antibodies). In some embodiments, hybridoma cells may be used as a preferred source of DNA. Once isolated, the DNA can be placed into expression vectors, which are then transfected into host cells. Host cells may include, but are not limited to HEK293 cells, HEK293T cells, simian COS cells, Chinese hamster ovary (CHO) cells, and myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567) or by covalently joining to the immunoglobulin coding sequence all or part of the coding sequence for a non-immunoglobulin polypeptide.

### Antibody Humanization

In some embodiments, anti-tau antibodies of the present disclosure may be prepared as humanized antibodies. “Humanized” antibodies are chimeric antibodies that contain minimal sequences (e.g., variable domains or CDRs) derived from non-human immunoglobulins (e.g., murine immunoglobulins). Humanized antibodies may be prepared from human (recipient) immunoglobulins in which residues from the hypervariable regions are replaced by hypervariable region residues from one or more non-human “donor” antibodies (e.g., mouse, rat, rabbit, or nonhuman primate). Donor antibodies may be selected based on desired specificity, affinity, and/or capacity. Humanized antibodies may include one or more back-mutation that includes the reversion of one or more amino acids back to amino acids found in a donor antibody. Conversely, residues from donor antibodies included in humanized antibodies may be mutated to match residues present in human recipient antibodies. Back-mutations may be introduced to reduce human immune response to the humanized antibodies. In some embodiments, back-mutations are introduced to avoid issues with antibody manufacturing (e.g., protein aggregation or post-translational modification).

For construction of expression plasmids encoding fully humanized antibodies with human constant regions, DNA sequences encoding antibody variable regions may be inserted into expression vectors (e.g., mammalian expression vectors) between an upstream promoter/enhancer and immunoglobulin signal sequence and a downstream immunoglobulin constant region gene. DNA samples may then be transfected into mammalian cells for antibody production. Constant domains from any class of human antibody may be used. There are five major classes of intact human antibodies: IgA, IgD, IgE, IgG, and IgM, and several of these may be further divided into subclasses (isotypes), e.g., IgG1 (human and murine), IgG2 (human), IgG2a (murine), IgG2b (murine), IgG2c (murine), IgG3 (human and murine), IgG4 (human), IgA (murine), IgA1 (human), and IgA2 (human).

Cell lines with stable transfection of DNA encoding humanized antibodies may be prepared and used to establish stable cell lines. Cell lines producing humanized antibodies may be expanded for expression of humanized antibodies that may be harvested and purified from cell culture media.

In some embodiments, humanized antibodies of the present disclosure may have cross-reactivity with non-human species. Species cross-reactivity may allow antibodies to be used in different animals for various purposes. For example, cross-reactive antibodies may be used in pre-clinical animal studies to provide information about antibody efficacy and/or toxicity. Non-human species may include, but are not limited to, mouse, rat, rabbit, dog, pig, goat, sheep, and nonhuman primates (e.g., Cynomolgus monkeys).

### Antibody Conjugates

In some embodiments, antibodies of the present disclosure may be or be prepared as antibody conjugates. As used herein, the term “conjugate” refers to any agent, cargo, or chemical moiety that is attached to a recipient entity or the process of attaching such an agent, cargo, or chemical moiety. As used herein, the term “antibody conjugate” refers to any antibody with an attached agent, cargo, or chemical moiety. Conjugates utilized to prepare antibody conjugates may include therapeutic agents. Such therapeutic agents may include drugs. Antibody conjugates that include a conjugated drug are referred to herein as “antibody drug conjugates.” Antibody drug conjugates may be used to direct conjugated drugs to specific targets based on the affinity of associated antibodies for proteins or epitopes associated with such targets. Such antibody drug conjugates may be used to localize biological activity associated with such conjugated drugs to targeted cells, tissues, organs, or other targeted entities. In some embodiments, conjugates utilized to prepare antibody conjugates include detectable labels. Antibodies may be conjugated with detectable labels for purposes of detection. Such detectable labels may include, but are not limited to, radioisotopes, fluorophores, chromophores, chemiluminescent compounds, enzymes, enzyme co-factors, dyes, metal ions, ligands, biotin, avidin, streptavidin, haptens, quantum dots, or any other detectable labels known in the art or described herein.

Conjugates may be attached to antibodies directly or via a linker. Direct attachment may be by covalent bonding or by non-covalent association (e.g., ionic bonds, hydrostatic bonds, hydrophobic bonds, hydrogen bonds, hybridization, etc.). Linkers used for conjugate attachment may include any chemical structure capable of connecting an antibody to a conjugate. In some embodiments, linkers include polymeric molecules (e.g., nucleic acids, polypeptides, polyethylene glycols, carbohydrates, lipids, or combinations thereof). Antibody conjugate linkers may be cleavable (e.g., Through Contact with an Enzyme, Change in pH, or Change in Temperature).

### Exemplary Anti-Tau Antibodies

In some embodiments, the anti-tau antibody comprises at least one antigen-binding domain, e.g., a variable region or antigen binding fragment thereof, from an antibody described herein, e.g., from V0004, V0009, V0022, V0023, V0024, or V0052, e.g., as described in Table 1 or 4, or a sequence substantially identical (e.g., having at least about 70%, 75%, 80%, 85%, 90%, 92%, 95%, 97%, 98%, or 99% sequence identity) to any of the aforesaid sequences. In some embodiments, the anti-tau antibody comprises at least one antigen-binding domain, e.g., a variable region or antigen binding fragment thereof, from an antibody described in WO 2021/211753, the contents of which are hereby incorporated by reference in its entirety, or a sequence substantially identical (e.g., having at least about 70%, 75%, 80%, 85%, 90%, 92%, 95%, 97%, 98%, or 99% sequence identity) to any of the antibody sequences disclosed in WO 2021/211753.

In some embodiments, the anti-tau antibody comprises a heavy chain variable region from an antibody described herein, e.g., chosen from V0004, V0009, V0022, V0023, V0024, or V0052, e.g., as described in Table 4, or a sequence substantially identical (e.g., having at least about 70%, 75%, 80%, 85%, 90%, 92%, 95%, 97%, 98%, or 99% sequence identity) to any of the aforesaid sequences. In some embodiments, the heavy chain variable region comprises an amino acid sequence having at least one, two, or three modifications (e.g., substitutions, e.g., conservative substitutions), but not more than 30, 20, or 10 modifications (e.g., substitutions, e.g., conservative substitutions) of an amino acid sequence of a heavy chain variable region provided in Table 4.

In some embodiments, the nucleotide sequence encoding the anti-tau antibody comprises the nucleotide sequence of a heavy chain variable region from an antibody described herein, e.g., chosen from V0004, V0009, V0022, V0023, V0024, or V0052, e.g., as described in Table 4, or a nucleotide sequence substantially identical (e.g., having at least about 70%, 75%, 80%, 85%, 90%, 92%, 95%, 97%, 98%, or 99% sequence identity) to any of the aforesaid sequences.

In some embodiments, the anti-tau antibody comprises a light chain variable region from an antibody described herein, e.g., chosen from V0004, V0009, V0022, V0023, V0024, or V0052, e.g., as described in Table 4, or a sequence substantially identical (e.g., having at least about 70%, 75%, 80%, 85%, 90%, 92%, 95%, 97%, 98%, or 99% sequence identity) to any of the aforesaid sequences. In some embodiments, the light chain variable region comprises an amino acid sequence having at least one, two, or three modifications (e.g., substitutions, e.g., conservative substitutions), but not more than 30, 20, or 10 modifications (e.g., substitutions, e.g., conservative substitutions) of an amino acid sequence of a light chain variable region provided in Table 4.

In some embodiments, the nucleotide sequence encoding the anti-tau antibody comprises the nucleotide sequence of a light chain variable region from an antibody described herein, e.g., chosen from V0004, V0009, V0022, V0023, V0024, or V0052, e.g., as described in Table 4, or a nucleotide sequence substantially identical (e.g., having at least about 70%, 75%, 80%, 85%, 90%, 92%, 95%, 97%, 98%, or 99% sequence identity) to any of the aforesaid sequences.

In some embodiments, the anti-tau antibody comprises a heavy chain variable region and a light chain variable region from an antibody described herein, e.g., chosen from V0004, V0009, V0022, V0023, V0024, or V0052, e.g., as described in Table 4, or a sequence substantially identical (e.g., having at least about 70%, 75%, 80%, 85%, 90%, 92%, 95%, 97%, 98%, or 99% sequence identity) to any of the aforesaid sequences. In some embodiments, the anti-tau antibody comprises a heavy chain variable region comprising an amino acid sequence having at least one, two, or three modifications (e.g., substitutions, e.g., conservative substitutions), but not more than 30, 20, or 10 modifications (e.g., substitutions, e.g., conservative substitutions) of an amino acid sequence of a heavy chain variable region provided in Table 4; and a light chain variable region comprising an amino acid sequence having at least one, two, or three modifications (e.g., substitutions, e.g., conservative substitutions), but not more than 30, 20, or 10 modifications (e.g., substitutions, e.g., conservative substitutions) of an amino acid sequence of a light chain variable region provided in Table 4.

In some embodiments, the anti-tau antibody comprises a heavy chain constant region, e.g., a human IgG1, IgG2, IgG3, or IgG4 constant regions, or a murine IgG1, IgG2A, IgG2B, IgG2C, or IgG3 constant regions. In some embodiments, the heavy chain constant comprises an amino acid sequence set forth in Table 5, or a sequence substantially identical (e.g., having at least about 70%, 75%, 80%, 85%, 90%, 92%, 95%, 97%, 98%, or 99% sequence identity) to any of the aforesaid sequences. In some embodiments, a nucleic acid encoding the heavy chain constant region comprises a nucleotide sequence set forth in Table 5, or a nucleotide sequence substantially identical (e.g., having at least about 70%, 75%, 80%, 85%, 90%, 92%, 95%, 97%, 98%, or 99% sequence identity) to any of the aforesaid sequences.

In some embodiments, the anti-tau antibody comprises a light chain constant region, e.g., a kappa light chain constant region, e.g., a human kappa or lambda light chain constant region or a murine kappa or lambda light chain constant region. In some embodiments, the light chain constant comprises an amino acid sequence set forth in Table 5, or a sequence substantially identical (e.g., having at least about 70%, 75%, 80%, 85%, 90%, 92%, 95%, 97%, 98%, or 99% sequence identity) to any of the aforesaid sequences. In some embodiments, nucleic acid encoding the light chain constant region comprises a nucleotide sequence set forth in Table 5, or a nucleotide sequence substantially identical (e.g., having at least about 70%, 75%, 80%, 85%, 90%, 92%, 95%, 97%, 98%, or 99% sequence identity) to any of the aforesaid sequences.

In some embodiments, the anti-tau antibody comprises a heavy chain constant region and a light chain constant region. In some embodiments, the heavy chain constant region and the light chain constant region comprise an amino acid sequence set forth in Table 5, or a sequence substantially identical (e.g., having at least about 80%, 85%, 90%, 92%, 95%, 97%, 98%, or 99% sequence identity) thereto. In some embodiments, the nucleotide sequence encoding the anti-tau antibody comprises the nucleotide sequence of a heavy chain constant region and the nucleotide sequence of a kappa or lambda light chain constant region. In some embodiments, the nucleotide sequence encoding the heavy chain constant region and light chain constant region comprise a nucleotide sequence set forth in Table 5, or a nucleotide sequence substantially identical (e.g., having at least about 80%, 85%, 90%, 92%, 95%, 97%, 98%, or 99% sequence identity) thereto.

In some embodiments, the anti-tau antibody comprises a heavy chain variable region and a constant region, a light chain variable region and a constant region, or both, comprising an amino acid sequence of Table 4 for variable region, and an amino acid sequence of Table 5 for constant region; or is encoded by a nucleic acid sequence of Table 4, and 5; or a sequence substantially identical (e.g., having at least about 80%, 85%, 90%, 92%, 95%, 97%, 98%, or 99% sequence identity) to any of the aforesaid sequences.

In some embodiments, the anti-tau antibody comprises at least one, two, or three complementarity determining regions (CDRs) from a heavy chain variable region comprising an amino acid sequence in Table 4, or is encoded by a nucleic acid sequence in Table 4; or a sequence substantially identical (e.g., having at least about 70%, 75%, 80%, 85%, 90%, 92%, 95%, 97%, 98%, or 99% sequence identity) to any of the aforesaid sequences. In some embodiments, one, two, three, four, five, or all of the CDRs have one, two, three, four, five or more changes, e.g., amino acid substitutions, insertions, or deletions, relative to the amino acid sequence shown in Table 4, or encoded by a nucleotide sequence shown in Table 4. In some embodiments, the encoded anti-tau antibody includes a substitution in a heavy chain CDR, e.g., one or more substitutions in a CDR1, CDR2 and/or CDR3 of the heavy chain.

In some embodiments, the anti-tau antibody comprises at least one, two, or three complementarity determining regions (CDRs) from a light chain variable region comprising an amino acid sequence in Table 4, or is encoded by a nucleic acid sequence in Table 4; or a sequence substantially identical (e.g., having at least about 80%, 85%, 90%, 92%, 95%, 97%, 98%, or 99% sequence identity) to any of the aforesaid sequences. In some embodiments, one, two, three, four, five, or all of the CDRs have one, two, three, four, five or more changes, e.g., amino acid substitutions, insertions, or deletions, relative to the amino acid sequence shown in Table 4, or encoded by a nucleotide sequence shown in Table 4. In some embodiments, the anti-tau antibody includes a substitution in a light chain CDR, e.g., one or more substitutions in a CDR1, CDR2 and/or CDR3 of the light chain.

In some embodiments, the anti-tau antibody comprises at least one, two, three, four, five or six CDRs (or collectively all of the CDRs) from a heavy and light chain variable region comprising an amino acid sequence shown in Table 4, or is encoded by a nucleotide sequence shown in Table 4. In some embodiments, one or more of the CDRs (or collectively all of the CDRs) have one, two, three, four, five, six or more changes, e.g., amino acid substitutions, insertions, or deletions, relative to the CDRs shown in Table 4, or encoded by a nucleotide sequence shown in Table 4.

In some embodiments, the anti-tau antibody comprises all three CDRs from a heavy chain variable region, all three CDRs from light chain variable region, or both (e.g., all six CDRs from a heavy chain variable region and a light chain variable region) comprising an amino acid sequence shown in Table 4, or is encoded by a nucleotide sequence shown in Table 4.

In some embodiments, an anti-tau antibody of the present disclosure may include CDRs identified through CDR analysis of variable domain sequences presented herein via co-crystallography with bound antigen; by computational assessments based on comparisons with other antibodies (e.g., see Strohl, W. R Therapeutic Antibody Engineering. Woodhead Publishing, Philadelphia PA. 2012. Ch. 3, p47-54); or Kabat, Chothia, Al-Lazikani, Lefranc, or Honegger numbering schemes, as described previously.

TABLE 4
Exemplary anti-tau antibodies

| | SEQ | | |
|---|---|---|---|
| Ab ID | ID NO | Description | Sequence |
| V0004 | 299 | HC CDR1 | DYTFTNY |
| | | (Chothia) | |
| | 343 | HC CDR2 | DPNSGG |
| | | (Chothia) | |
| | 395 | HC CDR3 | DFDV |
| | | (Chothia) | |
| | 1140 | HC CDR1 | NYWMH |
| | | (Kabat) | |
| | 1141 | HC CDR2 | RIDPNSGGTRYNEKFKN |
| | | (Kabat) | |
| | 395 | HC CDR3 | DFDV |
| | | (Kabat) | |
| | 1155 | HC CDR1 | DYTFTNYW |
| | | (IMGT) | |
| | 1156 | HC CDR2 | IDPNSGGT |
| | | (IMGT) | |
| | 1157 | HC CDR3 | AGDFDV |
| | | (IMGT) | |
| | 4 | VH | QVQLQQPGAELVKPGASVKLSCKASDYTFTNYWMHWVKQRPGR |
| | | | GLEWIGRIDPNSGGTRYNEKFKNKATLTVDKPSSTAYMHLSSL |
| | | | TSEDSAVYYCAGDFDVWGTGTTVTVSS |
| | 150 | DNA VH | CAGGTCCAACTGCAGCAGCCTGGGGCTGAGCTTGTGAAGCCTG |
| | | | GGGCTTCAGTGAAGCTGTCCTGCAAGGCTTCTGACTACACCTT |
| | | | CACCAACTACTGGATGCACTGGGTGAAGCAGAGGCCTGGACGA |
| | | | GGCCTTGAGTGGATAGGAAGGATTGATCCTAATAGTGGTGGTA |
| | | | CTAGGTACAATGAGAAGTTCAAGAACAAGGCCACACTGACTGT |
| | | | TGACAAACCCTCCAGCACAGCCTACATGCATCTCAGCAGCCTG |
| | | | ACATCTGAGGACTCTGCGGTCTATTATTGTGCAGGGGACTTCG |
| | | | ATGTCTGGGGCACAGGGACCACGGTCACCGTCTCCTCA |
| | 460 | LC CDR1 | RASGNIHNYLA |
| | | (Chothia) | |
| | 518 | LC CDR2 | NAKTLPD |
| | | (Chothia) | |
| | 557 | LC CDR3 | QHFWSTPLT |
| | | (Chothia) | |
| | 460 | LC CDR1 | RASGNIHNYLA |
| | | (Kabat) | |
| | 518 | LC CDR2 | NAKTLPD |
| | | (Kabat) | |
| | 557 | LC CDR3 | QHFWSTPLT |
| | | (Kabat) | |
| | 1158 | LC CDR1 | GNIHNY |
| | | (IMGT) | |
| | | LC CDR2 | NAK |
| | | (IMGT) | |
| | 557 | LC CDR3 | QHFWSTPLT |
| | | (IMGT) | |
| | 78 | VL | DIQMTQSPASLSASVGETVTITCRASGNIHNYLAWYQQRQGKS |
| | | | PQLLVYNAKTLPDGVPSRFSGSGSGTQYSLKINSLQPEDFGSY |
| | | | CCQHFWSTPLTFGAGTKLELK |
| | 224 | DNA VL | GACATCCAGATGACTCAGTCTCCAGCCTCCCTATCTGCATCTG |
| | | | TGGGAGAAACTGTCACCATCACATGTCGAGCAAGTGGGAATAT |
| | | | TCACAATTATTTAGCATGGTATCAGCAGAGACAGGGAAAATCT |
| | | | CCTCAGCTCCTGGTCTATAATGCAAAAACCTTACCAGATGGTG |
| | | | TGCCATCAAGGTTCAGTGGCAGTGGATCAGGAACACAATATTC |
| | | | TCTCAAGATCAACAGCCTGCAGCCTGAAGATTTTGGGAGTTAT |
| | | | TGCTGTCAACATTTTTGGAGTACTCCGCTCACGTTCGGTGCTG |
| | | | GGACCAAGTTGGAGCTGAAA |
| V0009 | 304 | HC CDR1 | GFTFTDY |
| | | (Chothia) | |
| | 347 | HC CDR2 | RNKAKGFT |
| | | (Chothia) | |
| | 400 | HC CDR3 | DINY |
| | | (Chothia) | |
| | 1142 | HC CDR1 | DYYMS |
| | | (Kabat) | |
| | 1143 | HC CDR2 | LIRNKAKGFTTEYSASVKG |
| | | (Kabat) | |
| | 400 | HC CDR3 | DINY |
| | | (Kabat) | |
| | 1160 | HC CDR1 | GFTFTDYY |
| | | (IMGT) | |
| | 1161 | HC CDR2 | IRNKAKGFTT |
| | | (IMGT) | |
| | 1162 | HC CDR3 | VRDINY |
| | | (IMGT) | |
| | 9 | VH | EVQLVESGGALVQPGGSLSLSCAASGFTFTDYYMSWVRQPPGK |
| | | | ALEWLALIRNKAKGFTTEYSASVKGRFTISRDNSQSILLFQMN |
| | | | DLRADDSATYYCVRDINYWGQGTTLTVSS |
| | 155 | DNA VH | GAGGTGCAGCTGGTGGAGTCTGGAGGAGCCTTGGTACAGCCTG |
| | | | GGGGTTCTCTGAGTCTCTCCTGTGCAGCTTCTGGATTCACCTT |
| | | | CACTGATTACTACATGAGCTGGGTCCGCCAGCCTCCAGGGAAG |
| | | | GCACTTGAGTGGCTGGCTTTGATTAGAAACAAAGCTAAAGGTT |
| | | | TCACAACAGAATACAGTGCATCTGTGAAGGGTCGGTTCACCAT |
| | | | CTCCAGAGATAATTCCCAAAGCATCCTCCTTTTTCAAATGAAT |
| | | | GACCTGAGAGCTGACGACAGTGCCACTTATTACTGTGTAAGAG |
| | | | ATATAAACTACTGGGGCCAAGGCACCACTCTCACAGTCTCCTC |
| | | | A |
| | 464 | LC CDR1 | KSSQSLLYSTNQENYLA |
| | | (Chothia) | |
| | 523 | LC CDR2 | WASSRES |
| | | Chothia) | |
| | 562 | LC CDR3 | QQYYSYPRT |
| | | (Chothia) | |
| | 464 | LC CDR1 | KSSQSLLYSTNQENYLA |
| | | (Kabat) | |
| | 523 | LC CDR2 | WASSRES |
| | | (Kabat) | |
| | 562 | LC CDR3 | QQYYSYPRT |
| | | (Kabat) | |
| | 1163 | LC CDR1 | QSLLYSTNQENY |
| | | (IMGT) | |
| | | LC CDR2 | WAS |
| | | (IMGT) | |
| | 562 | LC CDR3 | QQYYSYPRT |
| | | (IMGT) | |
| | 83 | VL | DIVMSQSPSSLAVSVGEKVTMSCKSSQSLLYSTNQENYLAWYQ |
| | | | QKPGQSPKLLIYWASSRESGVPDRFTGSGSGTDFTLTISSVKA |
| | | | EDLAVYYCQQYYSYPRTFGGGTKLEIK |
| | 229 | DNA VL | GACATTGTGATGTCACAGTCTCCATCCTCCCTAGCTGTGTCAG |
| | | | TTGGAGAGAAGGTTACTATGAGCTGCAAGTCCAGTCAGAGCCT |
| | | | TTTATATAGTACCAATCAAGAGAACTACTTGGCCTGGTACCAG |
| | | | CAGAAACCAGGGCAGTCTCCTAAACTGCTGATTTACTGGGCAT |
| | | | CCTCTAGGGAATCTGGGGTCCCTGATCGCTTTACAGGCAGTGG |
| | | | ATCTGGGACAGATTTCACTCTCACCATCAGCAGTGTGAAGGCT |
| | | | GAAGACCTGGCAGTTTATTACTGTCAGCAATATTATAGCTATC |
| | | | CTCGGACGTTCGGTGGAGGCACCAAGCTGGAAATCAAA |
| V0022 | 314 | HC CDR1 | GFTFTRY |
| | | (Chothia) | |
| | 341 | HC CDR2 | NPNNGG |
| | | (Chothia) | |
| | 410 | HC CDR3 | GTGTGAMDY |
| | | (Chothia) | |
| | 1144 | HC CDR1 | RYWMH |
| | | (Kabat) | |
| | 1145 | HC CDR2 | NINPNNGGTDFNEKFKN |
| | | (Kabat) | |
| | 410 | HC CDR3 | GTGTGAMDY |
| | | (Kabat) | |
| | 1165 | HC CDR1 | GFTFTRYW |
| | | (IMGT) | |
| | 1166 | HC CDR2 | INPNNGGT |
| | | (IMGT) | |
| | 1167 | HC CDR3 | ARGTGTGAMDY |
| | | (IMGT) | |
| | 64 | HC CDR1 | GFTFTRYWMH |
| | | (Alternative) | |
| | 1145 | HC CDR2 | NINPNNGGTDFNEKFKN |
| | | (Alternative) | |
| | 1167 | HC CDR3 | ARGTGTGAMDY |
| | | (Alternative) | |
| | 21 | VH | QVQLQQPGTELVKPGSSVNLSCKASGFTFTRYWMHWVKERPGH |
| | | | GLEWIGNINPNNGGTDFNEKFKNKATLTVHKSSTTVFIQLSSL |
| | | | TSEDSAVYYCARGTGTGAMDYWGQGTSVTVSS |
| | 167 | DNA VH | CAGGTCCAACTGCAGCAGCCTGGGACTGAACTGGTGAAGCCTG |
| | | | GGTCTTCAGTGAACCTGTCCTGCAAGGCTTCTGGCTTCACCTT |
| | | | CACCAGGTACTGGATGCACTGGGTGAAGGAGAGGCCTGGACAT |
| | | | GGCCTTGAGTGGATTGGAAATATTAATCCTAACAATGGTGGTA |
| | | | CTGACTTCAATGAGAAGTTCAAGAACAAGGCCACACTGACTGT |
| | | | ACACAAGTCCTCCACCACAGTCTTCATCCAACTCAGCAGCCTG |
| | | | ACATCTGAGGACTCTGCGGTCTATTATTGTGCAAGAGGAACTG |
| | | | GGACGGGAGCTATGGACTACTGGGGTCAAGGAACCTCAGTCAC |
| | | | CGTCTCCTCA |
| | 7 | DNA VH | caagtgcagctgcagcagcctggcaccgagctggtgaaacctg |
| | | | gatctagcgtgaatctgagctgcaaggccagcggctttacctt |
| | | | caccagatactggatgcactgggtcaaggaacggccaggccac |
| | | | ggcctggaatggatcggcaatatcaaccccaacaacggcggaa |
| | | | cagatttcaacgagaagttcaagaacaaggctacactgaccgt |
| | | | gcacaaaagctccaccaccgtgttcatccagctgagctctctg |
| | | | acaagcgaggacagcgccgtgtactattgtgccagaggcaccg |
| | | | gcaccggcgccatggactactggggccagggaacatctgtgac |
| | | | agtgtccagc |
| | 190 | DNA VH | CAAGTGCAGCTGCAGCAACCGGGCACAGAGCTGGTGAAGCCTG |
| | | | GCAGCAGCGTGAACCTGAGCTGCAAGGCTAGCGGCTTCACCTT |
| | | | CACAAGATACTGGATGCACTGGGTGAAGGAGAGACCTGGCCAC |
| | | | GGCCTGGAGTGGATCGGCAACATCAACCCTAACAACGGCGGCA |
| | | | CCGACTTCAACGAGAAGTTCAAGAACAAGGCCACCCTGACCGT |
| | | | GCACAAGAGCAGCACCACCGTGTTCATTCAGCTGAGCAGCCTG |
| | | | ACAAGCGAGGACAGCGCCGTGTACTACTGCGCTAGAGGCACCG |
| | | | GCACCGGCGCCATGGACTACTGGGGCCAAGGCACAAGCGTGAC |
| | | | CGTGTCGTCGGC |
| | 8 | Heavy Chain | QVQLQQPGTELVKPGSSVNLSCKASGFTFTRYWMHWVKERPGH |
| | | | GLEWIGNINPNNGGTDFNEKFKNKATLTVHKSSTTVFIQLSSL |
| | | | TSEDSAVYYCARGTGTGAMDYWGQGTSVTVSSAKTTPPSVYPL |
| | | | APGSAAQTNSMVTLGCLVKGYFPEPVTVTWNSGSLSSGVHTFP |
| | | | AVLQSDLYTLSSSVTVPSSTWPSETVTCNVAHPASSTKVDKKI |
| | | | VPRDCGCKPCICTVPEVSSVFIFPPKPKDVLTITLTPKVTCVV |
| | | | VDISKDDPEVQFSWFVDDVEVHTAQTQPREEQFNSTFRSVSEL |
| | | | PIMHQDWLNGKEFKCRVNSAAFPAPIEKTISKTKGRPKAPQVY |
| | | | TIPPPKEQMAKDKVSLTCMITDFFPEDITVEWQWNGQPAENYK |
| | | | NTQPIMDTDGSYFVYSKLNVQKSNWEAGNTFTCSVLHEGLHNH |
| | | | HTEKSLSHSPG |
| | 65 | Heavy Chain | QVQLQQPGTELVKPGSSVNLSCKASGFTFTRYWMHWVKERPGH |
| | | | GLEWIGNINPNNGGTDFNEKFKNKATLTVHKSSTTVFIQLSSL |
| | | | TSEDSAVYYCARGTGTGAMDYWGQGTSVTVSSASTKGPSVFPL |
| | | | APCSRSTSESTAALGCLVKDYFPEPVTVSWNSGALTSGVHTFP |
| | | | AVLQSSGLYSLSSVVTVPSSSLGTKTYTCNVDHKPSNTKVDKR |
| | | | VESKYGPPCPPCPAPEFLGGPSVFLFPPKPKDTLMISRTPEVT |
| | | | CVVVDVSQEDPEVQFNWYVDGVEVHNAKTKPREEQFNSTYRVV |
| | | | SVLTVLHQDWLNGKEYKCKVSNKGLPSSIEKTISKAKGQPREP |
| | | | QVYTLPPSQEEMTKNQVSLTCLVKGFYPSDIAVEWESNGQPEN |
| | | | NYKTTPPVLDSDGSFFLYSRLTVDKSRWQEGNVFSCSVMHEAL |
| | | | HNHYTQKSLSLSLGK |
| | 10 | DNA Heavy | caagtgcagctgcagcagcctggcaccgagctggtgaaacctg |
| | | Chain | gatctagcgtgaatctgagctgcaaggccagcggctttacctt |
| | | | caccagatactggatgcactgggtcaaggaacggccaggccac |
| | | | ggcctggaatggatcggcaatatcaaccccaacaacggcggaa |
| | | | cagatttcaacgagaagttcaagaacaaggctacactgaccgt |
| | | | gcacaaaagctccaccaccgtgttcatccagctgagctctctg |
| | | | acaagcgaggacagcgccgtgtactattgtgccagaggcaccg |
| | | | gcaccggcgccatggactactggggccagggaacatctgtgac |
| | | | agtgtccagcgccaaaacgacacccccatctgtctatccactg |
| | | | gcccctggatctgctgcccaaactaactccatggtgaccctgg |
| | | | gatgcctggtcaagggctatttccctgagccagtgacagtgac |
| | | | ctggaactctggatccctgtccagcggtgtgcacaccttccca |
| | | | gctgtcctgcagtctgacctctacactctgagcagctcagtga |
| | | | ctgtcccctccagcacctggcccagcgagaccgtcacctgcaa |
| | | | cgttgcccacccggccagcagcaccaaggtggacaagaaaatt |
| | | | gtgcccagggattgtggttgtaagccttgcatatgtacagtcc |
| | | | cagaagtatcatctgtcttcatcttccccccaaagcccaagga |
| | | | tgtgctcaccattactctgactcctaaggtcacgtgtgttgtg |
| | | | gtagacatcagcaaggatgatcccgaggtccagttcagctggt |
| | | | ttgtagatgatgtggaggtgcacacagctcagacgcaaccccg |
| | | | ggaggagcagttcaacagcactttccgctcagtcagtgaactt |
| | | | cccatcatgcaccaggactggctcaatggcaaggagttcaaat |
| | | | gcagggtcaacagtgcagctttccctgcccccatcgagaaaac |
| | | | catctccaaaaccaaaggcagaccgaaggctccacaggtgtac |
| | | | accattccacctcccaaggagcagatggccaaggataaagtca |
| | | | gtctgacctgcatgataacagacttcttccctgaagacattac |
| | | | tgtggagtggcagtggaatgggcagccagcggagaactacaag |
| | | | aacactcagcccatcatggacacagatggctcttacttcgtct |
| | | | acagcaagctcaatgtgcagaagagcaactgggaggcaggaaa |
| | | | tactttcacctgctctgtgttacatgagggcctgcacaaccac |
| | | | catactgagaagagcctctcccactctcctggt |
| | 191 | DNA Heavy | CAAGTGCAGCTGCAGCAACCGGGCACAGAGCTGGTGAAGCCTG |
| | | Chain | GCAGCAGCGTGAACCTGAGCTGCAAGGCTAGCGGCTTCACCTT |
| | | | CACAAGATACTGGATGCACTGGGTGAAGGAGAGACCTGGCCAC |
| | | | GGCCTGGAGTGGATCGGCAACATCAACCCTAACAACGGCGGCA |
| | | | CCGACTTCAACGAGAAGTTCAAGAACAAGGCCACCCTGACCGT |
| | | | GCACAAGAGCAGCACCACCGTGTTCATTCAGCTGAGCAGCCTG |
| | | | ACAAGCGAGGACAGCGCCGTGTACTACTGCGCTAGAGGCACCG |
| | | | GCACCGGCGCCATGGACTACTGGGGCCAAGGCACAAGCGTGAC |
| | | | CGTGTCGTCGGCTAGCACCAAGGGTCCTTCCGTGTTTCCTCTA |
| | | | GCCCCTTGCAGCAGAAGCACAAGCGAGAGCACCGCCGCCCTGG |
| | | | GCTGCCTGGTTAAAGATTATTTCCCTGAGCCTGTGACCGTGAG |
| | | | CTGGAATAGCGGCGCCCTGACAAGCGGCGTGCACACCTTCCCT |
| | | | GCCGTGCTGCAGAGCAGCGGCCTGTACAGCCTGAGCAGCGTGG |
| | | | TGACCGTGCCTTCTAGCAGCCTGGGTACTAAGACCTACACCTG |
| | | | CAACGTGGACCACAAGCCTAGCAACACCAAGGTGGACAAGAGA |
| | | | GTGGAGAGCAAGTACGGTCCTCCGTGTCCCCCGTGCCCTGCCC |
| | | | CTGAGTTCCTGGGCGGCCCTAGCGTTTTCTTGTTTCCACCTAA |
| | | | GCCTAAGGACACCCTGATGATCAGCAGAACCCCTGAGGTGACC |
| | | | TGCGTGGTGGTGGACGTGAGCCAAGAGGACCCTGAGGTGCAGT |
| | | | TCAACTGGTACGTGGACGGCGTGGAGGTGCACAACGCCAAGAC |
| | | | CAAGCCTAGAGAGGAGCAGTTCAACAGCACCTACAGAGTGGTG |
| | | | AGCGTGCTGACCGTGCTGCACCAAGACTGGCTGAACGGCAAGG |
| | | | AGTACAAGTGCAAGGTGAGCAACAAGGGCCTGCCTAGCAGTAT |
| | | | TGAAAAGACCATCAGCAAGGCCAAGGGACAGCCTAGAGAGCCT |
| | | | CAAGTGTACACCCTGCCTCCTAGCCAAGAGGAGATGACCAAGA |
| | | | ACCAAGTGAGCCTGACCTGCCTGGTGAAGGGGTTTTACCCTAG |
| | | | CGACATCGCCGTGGAGTGGGAGAGCAACGGACAGCCTGAGAAC |
| | | | AACTACAAGACCACCCCTCCTGTGCTGGACAGCGACGGCAGCT |
| | | | TCTTCCTGTACAGCAGACTGACCGTGGACAAGAGCAGATGGCA |
| | | | AGAGGGCAACGTGTTCAGCTGCAGCGTGATGCACGAGGCCCTG |
| | | | CACAACCACTACACACAGAAGAGCCTGAGCCTGAGCCTGGGCA |
| | | | AG |
| | 1154 | LC CDR1 | RSSQSLVHNNGITYLY |
| | | (Chothia) | |
| | 529 | LC CDR2 | RVSNRFS |
| | | (Chothia) | |
| | 571 | LC CDR3 | FQGTHVPRT |
| | | (Chothia) | |
| | 1146 | LC CDR1 | RSSQSLVHNNGITYLY |
| | | (Kabat) | |
| | 529 | LC CDR2 | RVSNRFS |
| | | (Kabat) | |
| | 571 | LC CDR3 | FQGTHVPRT |
| | | (Kabat) | |
| | 473 | LC CDR1 | QSLVHNNGITY |
| | | (IMGT) | |
| | | LC CDR2 | RVS |
| | | (IMGT) | |
| | 571 | LC CDR3 | FQGTHVPRT |
| | | (IMGT) | |
| | 1146 | LC CDR1 | RSSQSLVHNNGITYLY |
| | | (Alternative) | |
| | 529 | LC CDR2 | RVSNRFS |
| | | (Alternative) | |
| | 571 | LC CDR3 | FQGTHVPRT |
| | | (Alternative) | |
| | 93 | VL | DVVMTQTPLSLPVSLGDQASISCRSSQSLVHNNGITYLYWYLQ |
| | | | KPGQSPKLLIYRVSNRFSGVPDRFGGSGSGTDFTLKISRVEAE |
| | | | DLGVYFCFQGTHVPRTFGGGTKLEIK |
| | 11 | DNA VL | gatgtggtgatgacacagacccctctgagcctgcctgtgtccc |
| | | | tcggcgaccaggccagcatcagctgtagaagcagccaatctct |
| | | | ggtgcacaacaatggcatcacctacctgtactggtatctgcag |
| | | | aaacctggccagagccccaagctgctgatctaccgggtgtcca |
| | | | atcggttcagcggagtgccagatagatttggcggatctggcag |
| | | | cggcaccgacttcaccctgaagatctctagagtcgaggccgag |
| | | | gacctgggcgtgtacttctgcttccagggcacacacgtgccca |
| | | | gaaccttcggcggcggaacaaagctggaaatcaag |
| | 241 | DNA VL | GATGTTGTGATGACCCAAACTCCACTCTCCCTGCCTGTCAGTC |
| | | | TTGGAGATCAAGCTTCCATCTCTTGCAGATCTAGTCAGAGCCT |
| | | | TGTACACAACAATGGAATCACCTATTTATATTGGTACCTGCAG |
| | | | AAGCCAGGCCAGTCTCCAAAGCTCCTGATCTACAGGGTTTCCA |
| | | | ACCGATTTTCTGGGGTCCCAGACAGGTTCGGTGGCAGTGGATC |
| | | | AGGGACAGATTTCACACTCAAGATCAGCAGAGTGGAGGCTGAG |
| | | | GATCTGGGAGTTTATTTCTGCTTTCAAGGTACACATGTTCCTC |
| | | | GGACGTTCGGTGGAGGCACCAAGCTGGAAATCAAA |
| | 192 | DNA VL | GACGTGGTGATGACACAGACCCCTCTGAGCCTGCCTGTGAGCC |
| | | | TGGGCGACCAAGCTAGCATCAGCTGCAGAAGCTCTCAGAGCCT |
| | | | GGTGCACAACAACGGCATCACCTACCTGTACTGGTACCTGCAG |
| | | | AAGCCTGGACAGAGCCCTAAGCTGCTGATCTACAGAGTGAGCA |
| | | | ACAGATTCAGCGGCGTGCCTGACAGATTCGGCGGCAGCGGCAG |
| | | | CGGCACCGACTTCACCCTGAAGATCAGCAGAGTGGAGGCCGAG |
| | | | GACCTGGGCGTGTACTTCTGCTTCCAAGGCACGCATGTACCTA |
| | | | GAACCTTCGGCGGCGGCACCAAGCTGGAGATCAAG |
| | 12 | Light Chain | DVVMTQTPLSLPVSLGDQASISCRSSQSLVHNNGITYLYWYLQ |
| | | | KPGQSPKLLIYRVSNRFSGVPDRFGGSGSGTDFTLKISRVEAE |
| | | | DLGVYFCFQGTHVPRTFGGGTKLEIKRADAAPTVSIFPPSSEQ |
| | | | LTSGGASVVCFLNNFYPKDINVKWKIDGSERQNGVLNSWTDQD |
| | | | SKDSTYSMSSTLTLTKDEYERHNSYTCEATHKTSTSPIVKSFN |
| | | | RNEC |
| | 66 | Light Chain | DVVMTQTPLSLPVSLGDQASISCRSSQSLVHNNGITYLYWYLQ |
| | | | KPGQSPKLLIYRVSNRFSGVPDRFGGSGSGTDFTLKISRVEAE |
| | | | DLGVYFCFQGTHVPRTFGGGTKLEIKRTVAAPSVFIFPPSDEQ |
| | | | LKSGTASVVCLLNNFYPREAKVQWKVDNALQSGNSQESVTEQD |
| | | | SKDSTYSLSSTLTLSKADYEKHKVYACEVTHQGLSSPVTKSFN |
| | | | RGEC |
| | 13 | DNA Light | gatgtggtgatgacacagacccctctgagcctgcctgtgtccc |
| | | Chain | tcggcgaccaggccagcatcagctgtagaagcagccaatctct |
| | | | ggtgcacaacaatggcatcacctacctgtactggtatctgcag |
| | | | aaacctggccagagccccaagctgctgatctaccgggtgtcca |
| | | | atcggttcagcggagtgccagatagatttggcggatctggcag |
| | | | cggcaccgacttcaccctgaagatctctagagtcgaggccgag |
| | | | gacctgggcgtgtacttctgcttccagggcacacacgtgccca |
| | | | gaaccttcggcggcggaacaaagctggaaatcaagcgggctga |
| | | | tgctgcaccaactgtatccatcttcccaccatccagtgagcag |
| | | | ttaacatctggaggtgcctcagtcgtgtgcttcttgaacaact |
| | | | tctaccccaaagacatcaatgtcaagtggaagattgatggcag |
| | | | tgaacgacaaaatggcgtcctgaacagttggactgatcaggac |
| | | | agcaaagacagcacctacagcatgagcagcaccctcacgttga |
| | | | ccaaggacgagtatgaacgacataacagctatacctgtgaggc |
| | | | cactcacaagacatcaacttcacccattgtcaagagcttcaac |
| | | | aggaatgagtgt |
| | 193 | DNA Light | GACGTGGTGATGACACAGACCCCTCTGAGCCTGCCTGTGAGCC |
| | | Chain | TGGGCGACCAAGCTAGCATCAGCTGCAGAAGCTCTCAGAGCCT |
| | | | GGTGCACAACAACGGCATCACCTACCTGTACTGGTACCTGCAG |
| | | | AAGCCTGGACAGAGCCCTAAGCTGCTGATCTACAGAGTGAGCA |
| | | | ACAGATTCAGCGGCGTGCCTGACAGATTCGGCGGCAGCGGCAG |
| | | | CGGCACCGACTTCACCCTGAAGATCAGCAGAGTGGAGGCCGAG |
| | | | GACCTGGGCGTGTACTTCTGCTTCCAAGGCACGCATGTACCTA |
| | | | GAACCTTCGGCGGCGGCACCAAGCTGGAGATCAAGAGAACCGT |
| | | | GGCCGCCCCTAGCGTGTTCATCTTCCCTCCTAGCGACGAGCAG |
| | | | CTGAAGAGCGGCACCGCTAGCGTGGTGTGCCTGCTGAACAACT |
| | | | TCTACCCTAGAGAGGCCAAGGTGCAGTGGAAGGTGGACAACGC |
| | | | CCTGCAGAGCGGCAACAGCCAAGAGAGCGTGACCGAGCAAGAC |
| | | | AGCAAGGACAGCACCTACAGCCTGAGCAGCACCCTGACCCTGA |
| | | | GCAAGGCCGACTACGAGAAGCACAAGGTGTACGCCTGCGAGGT |
| | | | GACCCACCAAGGCCTGAGCAGCCCTGTGACCAAGAGCTTCAAC |
| | | | AGAGGCGAGTGC |
| V0023 | 315 | HC CDR1 | GYTFTIF |
| | | (Chothia) | |
| | 341 | HC CDR2 | NPNNGG |
| | | (Chothia) | |
| | 410 | HC CDR3 | GTGTGAMDY |
| | | (Chothia) | |
| | 1147 | HC CDR1 | IFWMH |
| | | (Kabat) | |
| | 1148 | HC CDR2 | KINPNNGGGDYNEKFKS |
| | | (Kabat) | |
| | 410 | HC CDR3 | GTGTGAMDY |
| | | (Kabat) | |
| | 1168 | HC CDR1 | GYTFTIFW |
| | | (IMGT) | |
| | 1169 | HC CDR2 | INPNNGGG |
| | | (IMGT) | |
| | 1167 | HC CDR3 | ARGTGTGAMDY |
| | | (IMGT) | |
| | 22 | VH | QVQLQQPGTELVKPGASVKLSCKASGYTFTIFWMHWVKQRPGH |
| | | | GLEWIGKINPNNGGGDYNEKFKSKATLTVDKSSTTAYLQLSSL |
| | | | TSEDSAVYYCARGTGTGAMDYWGQGTSVTVSS |
| | 168 | DNA VH | CAGGTCCAACTGCAGCAGCCTGGGACTGAACTGGTGAAGCCTG |
| | | | GGGCTTCAGTGAAGCTGTCCTGCAAGGCTTCTGGCTACACCTT |
| | | | CACCATCTTCTGGATGCACTGGGTGAAGCAGAGGCCTGGACAT |
| | | | GGCCTTGAGTGGATTGGAAAGATTAATCCTAACAATGGAGGTG |
| | | | GTGACTACAATGAGAAATTCAAGAGTAAGGCCACATTGACTGT |
| | | | AGACAAATCCTCCACCACAGCCTACTTGCAGCTCAGCAGCCTG |
| | | | ACATCTGAGGACTCTGCGGTCTATTATTGTGCAAGAGGAACTG |
| | | | GGACGGGAGCTATGGACTACTGGGGTCAAGGAACCTCAGTCAC |
| | | | CGTCTCCTCA |
| | 474 | LC CDR 1 | RSSQSLVHSNGITHLY |
| | | (Chothia) | |
| | 529 | LC CDR2 | RVSNRFS |
| | | (Chothia) | |
| | 571 | LC CDR3 | FQGTHVPRT |
| | | (Chothia) | |
| | 474 | LC CDR1 | RSSQSLVHSNGITHLY |
| | | (Kabat) | |
| | 529 | LC CDR2 | RVSNRFS |
| | | (Kabat) | |
| | 571 | LC CDR3 | FQGTHVPRT |
| | | (Kabat) | |
| | 1170 | LC CDR1 | QSLVHSNGITH |
| | | (IMGT) | |
| | | LC CDR2 | RVS |
| | | (IMGT) | |
| | 571 | LC CDR3 | FQGTHVPRT |
| | | (IMGT) | |
| | 94 | VL | DVVMTQTPLSLPVSLGDHASISCRSSQSLVHSNGITHLYWYLQ |
| | | | RPGQTPKLLIYRVSNRFSGVPDRFSGSGSGTDFTLKISSVEAE |
| | | | DLGVYFCFQGTHVPRTFGGGTKLEIE |
| | 242 | DNA VL | GATGTTGTGATGACCCAAACTCCACTCTCCCTGCCTGTCAGTC |
| | | | TTGGAGATCACGCTTCCATCTCTTGCAGATCTAGTCAGAGCCT |
| | | | TGTACACAGCAATGGAATCACCCATTTATATTGGTACCTGCAG |
| | | | AGGCCAGGCCAGACTCCAAAGCTCCTGATCTACAGGGTTTCCA |
| | | | ACCGATTTTCTGGGGTCCCAGACAGGTTCAGTGGCAGTGGATC |
| | | | AGGGACAGATTTCACACTCAAGATCAGCAGCGTGGAGGCTGAG |
| | | | GATCTGGGAGTTTATTTCTGCTTTCAAGGTACACATGTTCCTC |
| | | | GGACGTTCGGTGGAGGCACCAAGCTGGAAATCGAA |
| V0024 | 316 | HC CDR1 | GYTFTRF |
| | | (Chothia) | |
| | 341 | HC CDR2 | NPNNGG |
| | | (Chothia) | |
| | 410 | HC CDR3 | GTGTGAMDY |
| | | (Chothia) | |
| | 1149 | HC CDR1 | RFWMH |
| | | (Kabat) | |
| | 1150 | HC CDR2 | NINPNNGGTDNNERFKS |
| | | (Kabat) | |
| | 410 | HC CDR3 | GTGTGAMDY |
| | | (Kabat) | |
| | 1171 | HC CDR1 | GYTFTRFW |
| | | (IMGT) | |
| | 1166 | HC CDR2 | INPNNGGT |
| | | (IMGT) | |
| | 1167 | HC CDR3 | ARGTGTGAMDY |
| | | (IMGT) | |
| | 23 | VH | QVQLQQPGTELVKPGASVKLSCKASGYTFTRFWMHWVKQRPGQ |
| | | | GLEWIGNINPNNGGTDNNERFKSKATLTVDRSSSTAYMQLSSL |
| | | | TSEDSAVYYCARGTGTGAMDYWGQGTSVTVSS |
| | 169 | DNA VH | CAGGTCCAACTGCAGCAGCCTGGGACTGAACTGGTGAAGCCTG |
| | | | GGGCTTCAGTGAAGCTGTCCTGCAAGGCTTCTGGCTACACCTT |
| | | | CACCAGGTTCTGGATGCACTGGGTGAAGCAGAGGCCTGGACAA |
| | | | GGCCTTGAGTGGATTGGAAATATTAATCCTAACAATGGTGGTA |
| | | | CTGACAATAATGAGAGGTTCAAGAGCAAGGCCACACTGACTGT |
| | | | AGACAGATCCTCCAGCACAGCCTACATGCAGCTCAGCAGCCTG |
| | | | ACATCTGAGGACTCTGCGGTCTATTATTGTGCAAGAGGAACTG |
| | | | GGACGGGAGCTATGGACTACTGGGGTCAAGGAACCTCAGTCAC |
| | | | CGTCTCCTCA |
| | 475 | LC CDR 1 | RSSQSLVHSNGNTHLY |
| | | (Chothia) | |
| | 530 | LC CDR2 | RVSSRFS |
| | | (Chothia) | |
| | 571 | LC CDR3 | FQGTHVPRT |
| | | (Chothia) | |
| | 475 | LC CDR1 | RSSQSLVHSNGNTHLY |
| | | (Kabat) | |
| | 530 | LC CDR2 | RVSSRFS |
| | | (Kabat) | |
| | 571 | LC CDR3 | FQGTHVPRT |
| | | (Kabat) | |
| | 1172 | LC CDR1 | QSLVHSNGNTH |
| | | (IMGT) | |
| | | LC CDR2 | RVS |
| | | (IMGT) | |
| | 571 | LC CDR3 | FQGTHVPRT |
| | | (IMGT) | |
| | 95 | VL | DVVMTQTPLSLPVSLGDQASISCRSSQSLVHSNGNTHLYWYLQ |
| | | | KPGQSPKLLIYRVSSRFSGVPDRFSGSGSGTDFTLKISRVEAE |
| | | | DLGVYFCFQGTHVPRTFGGGTKLEIK |
| | 243 | DNA VL | GATGTGGTGATGACCCAAACTCCACTCTCCCTGCCTGTCAGTC |
| | | | TTGGAGATCAGGCTTCCATCTCTTGCAGATCTAGTCAGAGCCT |
| | | | TGTACACAGCAATGGAAACACCCATTTATATTGGTACCTGCAG |
| | | | AAGCCAGGCCAGTCTCCAAAGCTCCTGATCTACAGGGTTTCCA |
| | | | GCCGATTTTCTGGGGTCCCAGACAGGTTCAGTGGCAGTGGATC |
| | | | AGGGACAGATTTCACACTCAAGATCAGCAGAGTGGAGGCTGAG |
| | | | GATCTGGGAGTTTATTTCTGCTTTCAGGGTACACATGTTCCTC |
| | | | GGACGTTCGGTGGAGGCACCAAGCTGGAAATCAAA |
| V0052 | 325 | HC CDR1 | GFSLSTSAM |
| | | (Chothia) | |
| | 362 | HC CDR2 | YWDDD |
| | | (Chothia) | |
| | 435 | HC CDR3 | RRRGYGMDY |
| | | (Chothia) | |
| | 1152 | HC CDR1 | TSAMGVS |
| | | (Kabat) | |
| | 1153 | HC CDR2 | HIYWDDDKRYNPSLKS |
| | | (Kabat) | |
| | 435 | HC CDR3 | RRRGYGMDY |
| | | (Kabat) | |
| | 1173 | HC CDR1 | GFSLSTSAMG |
| | | (IMGT) | |
| | 1174 | HC CDR2 | IYWDDDK |
| | | (IMGT) | |
| | 1175 | HC CDR3 | ARRRRGYGMDY |
| | | (IMGT) | |
| | 51 | VH | QITLKESGPGILQSSQTLSLTCSFSGFSLSTSAMGVSWIRQPS |
| | | | GEGLEWLAHIYWDDDKRYNPSLKSRLTISKDTSRNQVFLKITS |
| | | | VDTADTATYYCARRRRGYGMDYWGQGTSVTVSS |
| | 197 | DNA VH | CAGATTACTCTGAAAGAGTCTGGCCCTGGGATATTGCAGTCCT |
| | | | CCCAGACCCTCAGTCTGACTTGTTCTTTCTCTGGGTTTTCACT |
| | | | GAGCACTTCTGCTATGGGTGTGAGTTGGATTCGTCAGCCTTCA |
| | | | GGAGAGGGTCTGGAGTGGCTGGCACACATTTACTGGGATGATG |
| | | | ACAAGCGCTATAACCCATCCCTGAAGAGCCGGCTCACAATCTC |
| | | | CAAGGATACCTCCAGAAACCAGGTATTCCTCAAGATCACCAGT |
| | | | GTGGACACTGCAGATACTGCCACATACTACTGTGCTCGAAGAA |
| | | | GGAGGGGGTATGGTATGGACTACTGGGGTCAAGGAACCTCAGT |
| | | | CACCGTCTCCTCA |
| | 495 | LC CDR 1 | KASQSVSNDVA |
| | | (Chothia) | |
| | 540 | LC CDR2 | YASNRCT |
| | | (Chothia) | |
| | 587 | LC CDR3 | QQDYRSPLT |
| | | (Chothia) | |
| | 495 | LC CDR1 | KASQSVSNDVA |
| | | (Kabat) | |
| | 540 | LC CDR2 | YASNRCT |
| | | (Kabat) | |
| | 587 | LC CDR3 | QQDYRSPLT |
| | | (Kabat) | |
| | 1176 | LC CDR1 | QSVSND |
| | | (IMGT) | |
| | | LC CDR2 | YAS |
| | | (IMGT) | |
| | 587 | LC CDR3 | QQDYRSPLT |
| | | (IMGT) | |
| | 122 | VL | SIVMTQTPKFLLVSAGDRVTITCKASQSVSNDVAWYQQKPGQS |
| | | | PKLLIYYASNRCTGVPDRFTGSGYGTDFTFTISTVQAEDLAVY |
| | | | FCQQDYRSPLTFGAGTKLELK |
| | 270 | DNA VL | AGTATTGTGATGACCCAGACTCCCAAATTCCTGCTTGTATCAG |
| | | | CAGGAGACAGGGTTACCATAACCTGCAAGGCCAGTCAGAGTGT |
| | | | GAGTAATGATGTAGCTTGGTACCAACAGAAGCCAGGGCAGTCT |
| | | | CCTAAACTGCTAATATACTATGCATCCAATCGCTGCACTGGAG |
| | | | TCCCTGATCGCTTCACTGGCAGTGGATATGGGACGGATTTCAC |
| | | | TTTCACCATCAGCACTGTACAGGCTGAAGACCTGGCAGTTTAT |
| | | | TTCTGTCAGCAGGATTATAGGTCTCCGCTCACGTTCGGTGCTG |
| | | | GGACCAAGCTGGAGCTGAAA |

TABLE 4A
Exemplary Humanized Heavy Chain Variable Regions and Heavy Chains of V0022

| | SEQ | |
|---|---|---|
| Description | ID NO | Sequence |
| VH1 | 67 | QVQLVESGAEVKKPGASVKVSCKASGFTFTRYWMHWVKERPGHGLEWMGNINP |
| | | NNGGTDFNEKFKNRVTITVHKSASTAYMELSSLRSEDTAVYYCARGTGTGAMD |
| | | YWGQGTTVTVSS |
| DNA VH1 | 156 | CAAGTGCAGCTGGTGGAGAGCGGCGCCGAGGTGAAGAAGCCTGGCGCTAGCGT |
| | | GAAGGTGAGCTGCAAGGCTAGCGGCTTCACCTTCACAAGATACTGGATGCACT |
| | | GGGTGAAGGAGAGACCTGGCCACGGCCTGGAGTGGATGGGCAACATCAACCCT |
| | | AACAACGGCGGCACCGACTTCAACGAGAAGTTCAAGAACAGAGTGACCATCAC |
| | | CGTGCACAAGAGCGCGTCGACCGCTTACATGGAGCTGAGCAGCCTGAGAAGCG |
| | | AGGACACCGCCGTGTACTACTGCGCTAGAGGCACCGGCACCGGCGCCATGGAC |
| | | TACTGGGGCCAAGGCACCACCGTGACCGTCTCGTCC |
| Heavy Chain 1 | 170 | QVQLVESGAEVKKPGASVKVSCKASGFTFTRYWMHWVKERPGHGLEWMGNINP |
| | | NNGGTDFNEKFKNRVTITVHKSASTAYMELSSLRSEDTAVYYCARGTGTGAMD |
| | | YWGQGTTVTVSSASTKGPSVFPLAPCSRSTSESTAALGCLVKDYFPEPVTVSW |
| | | NSGALTSGVHTFPAVLQSSGLYSLSSVVTVPSSSLGTKTYTCNVDHKPSNTKV |
| | | DKRVESKYGPPCPPCPAPEFLGGPSVFLFPPKPKDTLMISRTPEVTCVVVDVS |
| | | QEDPEVQFNWYVDGVEVHNAKTKPREEQFNSTYRVVSVLTVLHQDWLNGKEYK |
| | | CKVSNKGLPSSIEKTISKAKGQPREPQVYTLPPSQEEMTKNQVSLTCLVKGFY |
| | | PSDIAVEWESNGQPENNYKTTPPVLDSDGSFFLYSRLTVDKSRWQEGNVFSCS |
| | | VMHEALHNHYTQKSLSLSLGK |
| DNA Heavy | 180 | CAAGTGCAGCTGGTGGAGAGCGGCGCCGAGGTGAAGAAGCCTGGCGCTAGCGT |
| Chain 1 | | GAAGGTGAGCTGCAAGGCTAGCGGCTTCACCTTCACAAGATACTGGATGCACT |
| | | GGGTGAAGGAGAGACCTGGCCACGGCCTGGAGTGGATGGGCAACATCAACCCT |
| | | AACAACGGCGGCACCGACTTCAACGAGAAGTTCAAGAACAGAGTGACCATCAC |
| | | CGTGCACAAGAGCGCGTCGACCGCTTACATGGAGCTGAGCAGCCTGAGAAGCG |
| | | AGGACACCGCCGTGTACTACTGCGCTAGAGGCACCGGCACCGGCGCCATGGAC |
| | | TACTGGGGCCAAGGCACCACCGTGACCGTCTCGTCCGCTAGCACGAAAGGTCC |
| | | AAGCGTTTTCCCTCTAGCCCCTTGCAGCAGAAGCACAAGCGAGAGCACCGCCG |
| | | CCCTGGGCTGCCTTGTAAAAGATTACTTCCCTGAGCCTGTGACCGTGAGTTGG |
| | | AACAGCGGCGCCCTGACAAGCGGCGTGCACACCTTCCCTGCCGTGCTGCAGAG |
| | | CAGCGGCCTGTACAGCCTGAGCAGCGTGGTGACCGTGCCAAGCAGCAGCCTGG |
| | | GCACCAAGACCTACACCTGCAACGTGGACCACAAGCCTAGCAACACCAAGGTG |
| | | GACAAGAGAGTGGAGAGCAAGTACGGTCCTCCGTGTCCCCCGTGCCCTGCCCC |
| | | TGAGTTCCTGGGCGGCCCTTCGGTGTTTCTGTTTCCACCTAAGCCTAAGGACA |
| | | CCCTGATGATCAGCAGAACCCCTGAGGTGACCTGCGTGGTGGTGGACGTGAGC |
| | | CAAGAGGACCCTGAGGTGCAGTTCAACTGGTACGTGGACGGCGTGGAGGTGCA |
| | | CAACGCCAAGACCAAGCCTAGAGAGGAGCAGTTCAACAGCACCTACAGAGTGG |
| | | TGAGCGTGCTGACCGTGCTGCACCAAGACTGGCTGAACGGCAAGGAGTACAAG |
| | | TGCAAGGTGAGCAACAAGGGCCTGCCTAGTTCCATTGAGAAGACCATCAGCAA |
| | | GGCCAAGGGACAGCCTAGAGAGCCTCAAGTGTACACCCTGCCTCCTAGCCAAG |
| | | AGGAGATGACCAAGAACCAAGTGAGCCTGACCTGCCTGGTGAAGGGATTCTAC |
| | | CCTAGCGACATCGCCGTGGAGTGGGAGAGCAACGGACAGCCTGAGAACAACTA |
| | | CAAGACCACCCCTCCTGTGCTGGACAGCGACGGCAGCTTCTTCCTGTACAGCA |
| | | GACTGACCGTGGACAAGAGCAGATGGCAAGAGGGCAACGTGTTCAGCTGCAGC |
| | | GTGATGCACGAGGCCCTGCACAACCACTACACACAGAAGAGCCTGAGCCTGAG |
| | | CCTGGGCAAG |
| VH2 | 68 | QVQLVQSGAEVKKPGASVKVSCKASGFTFTRYWMHWVRERPGHGLEWIGNINP |
| | | NNGGTDFNEKFKNRVTMTVHKSISTAYMELSSLRSDDSAVYYCARGTGTGAMD |
| | | YWGQGTLVTVSS |
| DNA VH2 | 157 | CAAGTGCAGCTGGTGCAGAGCGGCGCCGAGGTGAAGAAGCCTGGCGCTAGCGT |
| | | GAAGGTGAGCTGCAAGGCTAGCGGCTTCACCTTCACAAGATACTGGATGCACT |
| | | GGGTGAGAGAGAGACCTGGCCACGGCCTGGAGTGGATCGGCAACATCAACCCT |
| | | AACAACGGCGGCACCGACTTCAACGAGAAGTTCAAGAACAGAGTGACCATGAC |
| | | CGTGCACAAGAGCATCAGCACCGCCTACATGGAGCTGAGCAGCCTGAGAAGCG |
| | | ACGACAGCGCCGTGTACTACTGCGCTAGAGGCACCGGCACCGGCGCCATGGAC |
| | | TACTGGGGCCAAGGCACCCTGGTGACGGTAAGCTCC |
| Heavy Chain 2 | 171 | QVQLVQSGAEVKKPGASVKVSCKASGFTFTRYWMHWVRERPGHGLEWIGNINP |
| | | NNGGTDFNEKFKNRVTMTVHKSISTAYMELSSLRSDDSAVYYCARGTGTGAMD |
| | | YWGQGTLVTVSSASTKGPSVFPLAPCSRSTSESTAALGCLVKDYFPEPVTVSW |
| | | NSGALTSGVHTFPAVLQSSGLYSLSSVVTVPSSSLGTKTYTCNVDHKPSNTKV |
| | | DKRVESKYGPPCPPCPAPEFLGGPSVFLFPPKPKDTLMISRTPEVTCVVVDVS |
| | | QEDPEVQFNWYVDGVEVHNAKTKPREEQFNSTYRVVSVLTVLHQDWLNGKEYK |
| | | CKVSNKGLPSSIEKTISKAKGQPREPQVYTLPPSQEEMTKNQVSLTCLVKGFY |
| | | PSDIAVEWESNGQPENNYKTTPPVLDSDGSFFLYSRLTVDKSRWQEGNVFSCS |
| | | VMHEALHNHYTQKSLSLSLGK |
| DNA Heavy | 181 | CAAGTGCAGCTGGTGCAGAGCGGCGCCGAGGTGAAGAAGCCTGGCGCTAGCGT |
| Chain 2 | | GAAGGTGAGCTGCAAGGCTAGCGGCTTCACCTTCACAAGATACTGGATGCACT |
| | | GGGTGAGAGAGAGACCTGGCCACGGCCTGGAGTGGATCGGCAACATCAACCCT |
| | | AACAACGGCGGCACCGACTTCAACGAGAAGTTCAAGAACAGAGTGACCATGAC |
| | | CGTGCACAAGAGCATCAGCACCGCCTACATGGAGCTGAGCAGCCTGAGAAGCG |
| | | ACGACAGCGCCGTGTACTACTGCGCTAGAGGCACCGGCACCGGCGCCATGGAC |
| | | TACTGGGGCCAAGGCACCCTGGTGACGGTAAGCTCCGCTAGCACCAAGGGTCC |
| | | TAGTGTATTTCCCCTAGCCCCTTGCAGCAGAAGCACAAGCGAGAGCACCGCCG |
| | | CCCTGGGCTGCTTGGTGAAGGACTACTTCCCTGAGCCTGTCACAGTGTCCTGG |
| | | AATAGCGGCGCCCTGACAAGCGGCGTGCACACCTTCCCTGCCGTGCTGCAGAG |
| | | CAGCGGCCTGTACAGCCTGAGCAGCGTGGTGACCGTGCCTTCGTCGAGCCTGG |
| | | GCACCAAGACCTACACCTGCAACGTGGACCACAAGCCTAGCAACACCAAGGTG |
| | | GACAAGAGAGTGGAGAGCAAGTACGGTCCTCCGTGTCCCCCGTGCCCTGCCCC |
| | | TGAGTTCCTGGGCGGCCCTAGCGTGTTCCTATTTCCACCTAAGCCTAAGGACA |
| | | CCCTGATGATCAGCAGAACCCCTGAGGTGACCTGCGTGGTGGTGGACGTGAGC |
| | | CAAGAGGACCCTGAGGTGCAGTTCAACTGGTACGTGGACGGCGTGGAGGTGCA |
| | | CAACGCCAAGACCAAGCCTAGAGAGGAGCAGTTCAACAGCACCTACAGAGTGG |
| | | TGAGCGTGCTGACCGTGCTGCACCAAGACTGGCTGAACGGCAAGGAGTACAAG |
| | | TGCAAGGTGAGCAACAAGGGCCTGCCTAGCAGTATCGAGAAGACCATCAGCAA |
| | | GGCCAAGGGACAGCCTAGAGAGCCTCAAGTGTACACCCTGCCTCCTAGCCAAG |
| | | AGGAGATGACCAAGAACCAAGTGAGCCTGACCTGCCTGGTAAAAGGTTTCTAC |
| | | CCTAGCGACATCGCCGTGGAGTGGGAGAGCAACGGACAGCCTGAGAACAACTA |
| | | CAAGACCACCCCTCCTGTGCTGGACAGCGACGGCAGCTTCTTCCTGTACAGCA |
| | | GACTGACCGTGGACAAGAGCAGATGGCAAGAGGGCAACGTGTTCAGCTGCAGC |
| | | GTGATGCACGAGGCCCTGCACAACCACTACACACAGAAGAGCCTGAGCCTGAG |
| | | CCTGGGCAAG |
| VH3 | 69 | QVQLVQSGAEVKKSGASVKVSCKASGFTFTRYWMHWVRQAPGQGLEWIGNINP |
| | | NNGGTDFNEKFKNRVTLIRDTSTTTVFIQLTSLTSEDSAVYYCARGTGTGAMD |
| | | YWGQGTLVTVSS |
| DNA VH3 | 158 | CAAGTGCAGCTGGTGCAGAGCGGCGCCGAGGTGAAGAAGAGCGGCGCTAGCGT |
| | | GAAGGTGAGCTGCAAGGCTAGCGGCTTCACCTTCACAAGATACTGGATGCACT |
| | | GGGTGAGACAAGCCCCTGGCCAAGGCCTGGAGTGGATCGGCAACATCAACCCT |
| | | AACAACGGCGGCACCGACTTCAACGAGAAGTTCAAGAACAGAGTGACCCTGAT |
| | | CAGAGACACAAGCACCACCACCGTGTTCATTCAGCTGACAAGCCTGACAAGCG |
| | | AGGACAGCGCCGTGTACTACTGCGCTAGAGGCACCGGCACCGGCGCCATGGAC |
| | | TACTGGGGCCAAGGCACCCTGGTGACGGTAAGCTCC |
| Heavy Chain 3 | 172 | QVQLVQSGAEVKKSGASVKVSCKASGFTFTRYWMHWVRQAPGQGLEWIGNINP |
| | | NNGGTDFNEKFKNRVTLIRDTSTTTVFIQLTSLTSEDSAVYYCARGTGTGAMD |
| | | YWGQGTLVTVSSASTKGPSVFPLAPCSRSTSESTAALGCLVKDYFPEPVTVSW |
| | | NSGALTSGVHTFPAVLQSSGLYSLSSVVTVPSSSLGTKTYTCNVDHKPSNTKV |
| | | DKRVESKYGPPCPPCPAPEFLGGPSVFLFPPKPKDTLMISRTPEVTCVVVDVS |
| | | QEDPEVQFNWYVDGVEVHNAKTKPREEQFNSTYRVVSVLTVLHQDWLNGKEYK |
| | | CKVSNKGLPSSIEKTISKAKGQPREPQVYTLPPSQEEMTKNQVSLTCLVKGFY |
| | | PSDIAVEWESNGQPENNYKTTPPVLDSDGSFFLYSRLTVDKSRWQEGNVFSCS |
| | | VMHEALHNHYTQKSLSLSLGK |
| DNA Heavy | 182 | CAAGTGCAGCTGGTGCAGAGCGGCGCCGAGGTGAAGAAGAGCGGCGCTAGCGT |
| Chain 3 | | GAAGGTGAGCTGCAAGGCTAGCGGCTTCACCTTCACAAGATACTGGATGCACT |
| | | GGGTGAGACAAGCCCCTGGCCAAGGCCTGGAGTGGATCGGCAACATCAACCCT |
| | | AACAACGGCGGCACCGACTTCAACGAGAAGTTCAAGAACAGAGTGACCCTGAT |
| | | CAGAGACACAAGCACCACCACCGTGTTCATTCAGCTGACAAGCCTGACAAGCG |
| | | AGGACAGCGCCGTGTACTACTGCGCTAGAGGCACCGGCACCGGCGCCATGGAC |
| | | TACTGGGGCCAAGGCACCCTGGTGACGGTAAGCTCCGCTAGCACCAAGGGTCC |
| | | TAGTGTATTTCCCCTAGCCCCTTGCAGCAGAAGCACAAGCGAGAGCACCGCCG |
| | | CCCTGGGCTGCTTGGTGAAGGACTACTTCCCTGAGCCTGTCACAGTGTCCTGG |
| | | AATAGCGGCGCCCTGACAAGCGGCGTGCACACCTTCCCTGCCGTGCTGCAGAG |
| | | CAGCGGCCTGTACAGCCTGAGCAGCGTGGTGACCGTGCCTTCGTCGAGCCTGG |
| | | GCACCAAGACCTACACCTGCAACGTGGACCACAAGCCTAGCAACACCAAGGTG |
| | | GACAAGAGAGTGGAGAGCAAGTACGGTCCTCCGTGTCCCCCGTGCCCTGCCCC |
| | | TGAGTTCCTGGGCGGCCCTAGCGTGTTCCTATTTCCACCTAAGCCTAAGGACA |
| | | CCCTGATGATCAGCAGAACCCCTGAGGTGACCTGCGTGGTGGTGGACGTGAGC |
| | | CAAGAGGACCCTGAGGTGCAGTTCAACTGGTACGTGGACGGCGTGGAGGTGCA |
| | | CAACGCCAAGACCAAGCCTAGAGAGGAGCAGTTCAACAGCACCTACAGAGTGG |
| | | TGAGCGTGCTGACCGTGCTGCACCAAGACTGGCTGAACGGCAAGGAGTACAAG |
| | | TGCAAGGTGAGCAACAAGGGCCTGCCTAGCAGTATCGAGAAGACCATCAGCAA |
| | | GGCCAAGGGACAGCCTAGAGAGCCTCAAGTGTACACCCTGCCTCCTAGCCAAG |
| | | AGGAGATGACCAAGAACCAAGTGAGCCTGACCTGCCTGGTAAAAGGTTTCTAC |
| | | CCTAGCGACATCGCCGTGGAGTGGGAGAGCAACGGACAGCCTGAGAACAACTA |
| | | CAAGACCACCCCTCCTGTGCTGGACAGCGACGGCAGCTTCTTCCTGTACAGCA |
| | | GACTGACCGTGGACAAGAGCAGATGGCAAGAGGGCAACGTGTTCAGCTGCAGC |
| | | GTGATGCACGAGGCCCTGCACAACCACTACACACAGAAGAGCCTGAGCCTGAG |
| | | CCTGGGCAAG |
| VH4 | 70 | EVQLVQSGAEVKKPGSSVKVSCKASGFTFTRYWMHWVKERPGHGLEWIGNINP |
| | | NNGGTDFNEKFKNRVTMTVHKSITTAYMELSRLTSDDSAVYYCARGTGTGAMD |
| | | YWGQGTLVSVSS |
| DNA VH4 | 159 | GAGGTGCAGCTGGTGCAGAGCGGCGCCGAGGTGAAGAAGCCTGGCAGCAGCGT |
| | | GAAGGTGAGCTGCAAGGCTAGCGGCTTCACCTTCACAAGATACTGGATGCACT |
| | | GGGTGAAGGAGAGACCTGGCCACGGCCTGGAGTGGATCGGCAACATCAACCCT |
| | | AACAACGGCGGCACCGACTTCAACGAGAAGTTCAAGAACAGAGTGACCATGAC |
| | | CGTGCACAAGAGCATCACCACCGCCTACATGGAGCTGAGCCGGTTAACCTCCG |
| | | ACGACAGCGCCGTGTACTACTGCGCTAGAGGCACCGGCACCGGCGCCATGGAC |
| | | TACTGGGGCCAAGGCACCCTGGTGAGCGTGAGCAGC |
| Heavy Chain 4 | 173 | EVQLVQSGAEVKKPGSSVKVSCKASGFTFTRYWMHWVKERPGHGLEWIGNINP |
| | | NNGGTDFNEKFKNRVTMTVHKSITTAYMELSRLTSDDSAVYYCARGTGTGAMD |
| | | YWGQGTLVSVSSASTKGPSVFPLAPCSRSTSESTAALGCLVKDYFPEPVTVSW |
| | | NSGALTSGVHTFPAVLQSSGLYSLSSVVTVPSSSLGTKTYTCNVDHKPSNTKV |
| | | DKRVESKYGPPCPPCPAPEFLGGPSVFLFPPKPKDTLMISRTPEVTCVVVDVS |
| | | QEDPEVQFNWYVDGVEVHNAKTKPREEQFNSTYRVVSVLTVLHQDWLNGKEYK |
| | | CKVSNKGLPSSIEKTISKAKGQPREPQVYTLPPSQEEMTKNQVSLTCLVKGFY |
| | | PSDIAVEWESNGQPENNYKTTPPVLDSDGSFFLYSRLTVDKSRWQEGNVFSCS |
| | | VMHEALHNHYTQKSLSLSLGK |
| DNA Heavy | 183 | GAGGTGCAGCTGGTGCAGAGCGGCGCCGAGGTGAAGAAGCCTGGCAGCAGCGT |
| Chain 4 | | GAAGGTGAGCTGCAAGGCTAGCGGCTTCACCTTCACAAGATACTGGATGCACT |
| | | GGGTGAAGGAGAGACCTGGCCACGGCCTGGAGTGGATCGGCAACATCAACCCT |
| | | AACAACGGCGGCACCGACTTCAACGAGAAGTTCAAGAACAGAGTGACCATGAC |
| | | CGTGCACAAGAGCATCACCACCGCCTACATGGAGCTGAGCCGGTTAACCTCCG |
| | | ACGACAGCGCCGTGTACTACTGCGCTAGAGGCACCGGCACCGGCGCCATGGAC |
| | | TACTGGGGCCAAGGCACCCTGGTGAGCGTGAGCAGCGCTAGCACCAAGGGTCC |
| | | TAGTGTATTTCCCCTAGCCCCTTGCAGCAGAAGCACAAGCGAGAGCACCGCCG |
| | | CCCTGGGCTGCTTGGTGAAGGACTACTTCCCTGAGCCTGTGACCGTGAGCTGG |
| | | AACAGCGGCGCCCTGACAAGCGGCGTGCACACCTTCCCTGCCGTGCTGCAGAG |
| | | CAGCGGCCTGTACAGCCTGAGCAGCGTGGTGACCGTGCCTTCGTCGAGCCTGG |
| | | GCACCAAGACCTACACCTGCAACGTGGACCACAAGCCTAGCAACACCAAGGTG |
| | | GACAAGAGAGTGGAGAGCAAGTACGGTCCTCCGTGTCCCCCGTGCCCTGCCCC |
| | | TGAGTTCCTGGGCGGCCCTAGCGTGTTCCTATTTCCACCTAAGCCTAAGGACA |
| | | CCCTGATGATCAGCAGAACCCCTGAGGTGACCTGCGTGGTGGTGGACGTGAGC |
| | | CAAGAGGACCCTGAGGTGCAGTTCAACTGGTACGTGGACGGCGTGGAGGTGCA |
| | | CAACGCCAAGACCAAGCCTAGAGAGGAGCAGTTCAACAGCACCTACAGAGTGG |
| | | TGAGCGTGCTGACCGTGCTGCACCAAGACTGGCTGAACGGCAAGGAGTACAAG |
| | | TGCAAGGTGAGCAACAAGGGCCTGCCTAGCAGTATCGAGAAGACCATCAGCAA |
| | | GGCCAAGGGACAGCCTAGAGAGCCTCAAGTGTACACCCTGCCTCCTAGCCAAG |
| | | AGGAGATGACCAAGAACCAAGTGAGCCTGACCTGCCTGGTAAAAGGTTTCTAC |
| | | CCTAGCGACATCGCCGTGGAGTGGGAGAGCAACGGACAGCCTGAGAACAACTA |
| | | CAAGACCACCCCTCCTGTGCTGGACAGCGACGGCAGCTTCTTCCTGTACTCCC |
| | | GCCTGACGGTTGACAAGAGCAGATGGCAAGAGGGCAACGTGTTCAGCTGCAGC |
| | | GTGATGCACGAGGCCCTGCACAACCACTACACACAGAAGAGCCTGAGCCTGAG |
| | | CCTGGGCAAG |
| VH5 | 71 | QVQLVQSGAEVKKPGASVKVSCKASGFTFTRYWMHWVKERPGQGLEWMGNINP |
| | | NNGGTDFNEKFKNRVTMTVHKSTSTVFIQLSSLRSEDTAVYYCARGTGTGAMD |
| | | YWGQGTSVTVSS |
| DNA VH5 | 160 | CAAGTGCAGCTGGTGCAGAGCGGCGCCGAGGTGAAGAAGCCTGGCGCTAGCGT |
| | | GAAGGTGAGCTGCAAGGCTAGCGGCTTCACCTTCACAAGATACTGGATGCACT |
| | | GGGTGAAGGAGAGACCTGGCCAAGGCCTGGAGTGGATGGGCAACATCAACCCT |
| | | AACAACGGCGGCACCGACTTCAACGAGAAGTTCAAGAACAGAGTGACCATGAC |
| | | CGTGCACAAGAGCACAAGCACCGTGTTCATTCAGCTGAGCAGCCTGAGAAGCG |
| | | AGGACACCGCCGTGTACTACTGCGCTAGAGGCACCGGCACCGGCGCCATGGAC |
| | | TACTGGGGCCAAGGCACAAGCGTGACGGTAAGCTCC |
| Heavy Chain 5 | 174 | QVQLVQSGAEVKKPGASVKVSCKASGFTFTRYWMHWVKERPGQGLEWMGNINP |
| | | NNGGTDFNEKFKNRVTMTVHKSTSTVFIQLSSLRSEDTAVYYCARGTGTGAMD |
| | | YWGQGTSVTVSSASTKGPSVFPLAPCSRSTSESTAALGCLVKDYFPEPVTVSW |
| | | NSGALTSGVHTFPAVLQSSGLYSLSSVVTVPSSSLGTKTYTCNVDHKPSNTKV |
| | | DKRVESKYGPPCPPCPAPEFLGGPSVFLFPPKPKDTLMISRTPEVTCVVVDVS |
| | | QEDPEVQFNWYVDGVEVHNAKTKPREEQFNSTYRVVSVLTVLHQDWLNGKEYK |
| | | CKVSNKGLPSSIEKTISKAKGQPREPQVYTLPPSQEEMTKNQVSLTCLVKGFY |
| | | PSDIAVEWESNGQPENNYKTTPPVLDSDGSFFLYSRLTVDKSRWQEGNVFSCS |
| | | VMHEALHNHYTQKSLSLSLGK |
| DNA Heavy | 184 | CAAGTGCAGCTGGTGCAGAGCGGCGCCGAGGTGAAGAAGCCTGGCGCTAGCGT |
| Chain 5 | | GAAGGTGAGCTGCAAGGCTAGCGGCTTCACCTTCACAAGATACTGGATGCACT |
| | | GGGTGAAGGAGAGACCTGGCCAAGGCCTGGAGTGGATGGGCAACATCAACCCT |
| | | AACAACGGCGGCACCGACTTCAACGAGAAGTTCAAGAACAGAGTGACCATGAC |
| | | CGTGCACAAGAGCACAAGCACCGTGTTCATTCAGCTGAGCAGCCTGAGAAGCG |
| | | AGGACACCGCCGTGTACTACTGCGCTAGAGGCACCGGCACCGGCGCCATGGAC |
| | | TACTGGGGCCAAGGCACAAGCGTGACGGTAAGCTCCGCTAGCACCAAGGGTCC |
| | | TAGTGTATTTCCCCTAGCCCCTTGCAGCAGAAGCACAAGCGAGAGCACCGCCG |
| | | CCCTGGGCTGCTTGGTGAAGGACTACTTCCCTGAGCCTGTCACAGTGTCCTGG |
| | | AATAGCGGCGCCCTGACAAGCGGCGTGCACACCTTCCCTGCCGTGCTGCAGAG |
| | | CAGCGGCCTGTACAGCCTGAGCAGCGTGGTGACCGTGCCTTCGTCGAGCCTGG |
| | | GCACCAAGACCTACACCTGCAACGTGGACCACAAGCCTAGCAACACCAAGGTG |
| | | GACAAGAGAGTGGAGAGCAAGTACGGTCCTCCGTGTCCCCCGTGCCCTGCCCC |
| | | TGAGTTCCTGGGCGGCCCTAGCGTGTTCCTATTTCCACCTAAGCCTAAGGACA |
| | | CCCTGATGATCAGCAGAACCCCTGAGGTGACCTGCGTGGTGGTGGACGTGAGC |
| | | CAAGAGGACCCTGAGGTGCAGTTCAACTGGTACGTGGACGGCGTGGAGGTGCA |
| | | CAACGCCAAGACCAAGCCTAGAGAGGAGCAGTTCAACAGCACCTACAGAGTGG |
| | | TGAGCGTGCTGACCGTGCTGCACCAAGACTGGCTGAACGGCAAGGAGTACAAG |
| | | TGCAAGGTGAGCAACAAGGGCCTGCCTAGCAGTATCGAGAAGACCATCAGCAA |
| | | GGCCAAGGGACAGCCTAGAGAGCCTCAAGTGTACACCCTGCCTCCTAGCCAAG |
| | | AGGAGATGACCAAGAACCAAGTGAGCCTGACCTGCCTGGTAAAAGGTTTCTAC |
| | | CCTAGCGACATCGCCGTGGAGTGGGAGAGCAACGGACAGCCTGAGAACAACTA |
| | | CAAGACCACCCCTCCTGTGCTGGACAGCGACGGCAGCTTCTTCCTGTACAGCA |
| | | GACTGACCGTGGACAAGAGCAGATGGCAAGAGGGCAACGTGTTCAGCTGCAGC |
| | | GTGATGCACGAGGCCCTGCACAACCACTACACACAGAAGAGCCTGAGCCTGAG |
| | | CCTGGGCAAG |

TABLE 4B
Exemplary Humanized Light Chain Variable Regions and Light Chains of V0022

| | SEQ | |
|---|---|---|
| Description | ID NO | Sequence |
| VL1 | 72 | DVVMTQSPLSLPVTLGQPASISCRSSQSLVHNNGITYLYWYQQRPGQSPRLLI |
| | | YRVSNRFSGVPDRFSGSGSGTDFTLKISRVEAEDVGVYYCFQGTHVPRTFGQG |
| | | TKLEIK |
| DNA VL1 | 161 | GACGTGGTGATGACACAGAGCCCTCTGAGCCTGCCTGTGACCCTGGGACAGCC |
| | | TGCTAGCATCAGCTGCAGAAGCTCTCAGAGCCTGGTGCACAACAACGGCATCA |
| | | CCTACCTGTACTGGTATCAGCAGAGACCTGGACAGAGCCCTAGACTGCTGATC |
| | | TACAGAGTGAGCAACAGATTCAGCGGCGTGCCTGACAGATTCTCCGGCTCCGG |
| | | CAGCGGCACCGACTTCACCCTGAAGATCAGCAGAGTGGAGGCCGAGGACGTGG |
| | | GCGTGTACTACTGCTTTCAAGGCACCCATGTCCCTAGAACCTTCGGCCAAGGC |
| | | ACCAAGCTGGAGATCAAG |
| Light Chain 1 | 175 | DVVMTQSPLSLPVTLGQPASISCRSSQSLVHNNGITYLYWYQQRPGQSPRLLI |
| | | YRVSNRFSGVPDRFSGSGSGTDFTLKISRVEAEDVGVYYCFQGTHVPRTFGQG |
| | | TKLEIKRTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGLSSPVTK |
| | | SFNRGEC |
| DNA Light | 185 | GACGTGGTGATGACACAGAGCCCTCTGAGCCTGCCTGTGACCCTGGGACAGCC |
| Chain 1 | | TGCTAGCATCAGCTGCAGAAGCTCTCAGAGCCTGGTGCACAACAACGGCATCA |
| | | CCTACCTGTACTGGTATCAGCAGAGACCTGGACAGAGCCCTAGACTGCTGATC |
| | | TACAGAGTGAGCAACAGATTCAGCGGCGTGCCTGACAGATTCTCCGGCTCCGG |
| | | CAGCGGCACCGACTTCACCCTGAAGATCAGCAGAGTGGAGGCCGAGGACGTGG |
| | | GCGTGTACTACTGCTTTCAAGGCACCCATGTCCCTAGAACCTTCGGCCAAGGC |
| | | ACCAAGCTGGAGATCAAGAGAACCGTGGCCGCCCCTAGCGTGTTCATCTTCCC |
| | | TCCTAGCGACGAGCAGCTGAAGAGCGGCACCGCTAGCGTGGTGTGCCTGCTGA |
| | | ACAACTTCTACCCTAGAGAGGCCAAGGTGCAGTGGAAGGTGGACAACGCCCTG |
| | | CAGAGCGGCAACAGCCAAGAGAGCGTGACCGAGCAAGACAGCAAGGACAGCAC |
| | | CTACAGCCTGAGCAGCACCCTGACCCTGAGCAAGGCCGACTACGAGAAGCACA |
| | | AGGTGTACGCCTGCGAGGTGACCCACCAAGGCCTGAGCAGCCCTGTGACCAAG |
| | | AGCTTCAACAGAGGCGAGTGC |
| VL2 | 73 | DIVMTQSPLSLSVTPGQPASISCRSSQSLVHNNGITYLYWYLQKPGQSPQLLI |
| | | YRVSNRFSGVPDRFSGSGSGTDFTLKISRVEAEDVGVYYCFQGTHVPRTFGQG |
| | | TKVEIK |
| DNA VL2 | 162 | GACATCGTGATGACACAGAGCCCTCTGAGCCTGAGCGTGACCCCTGGACAGCC |
| | | TGCTAGCATCAGCTGCAGAAGCTCTCAGAGCCTGGTGCACAACAACGGCATCA |
| | | CCTACCTGTACTGGTACCTGCAGAAGCCTGGACAGAGCCCTCAGCTGCTGATC |
| | | TACAGAGTGAGCAACAGATTCAGCGGCGTGCCTGACAGATTCTCCGGCTCCGG |
| | | CAGCGGCACCGACTTCACCCTGAAGATCAGCAGAGTGGAGGCCGAGGACGTGG |
| | | GCGTGTACTACTGCTTTCAAGGCACCCATGTCCCTAGAACCTTCGGCCAAGGC |
| | | ACCAAGGTGGAGATCAAG |
| Light Chain 2 | 176 | DIVMTQSPLSLSVTPGQPASISCRSSQSLVHNNGITYLYWYLQKPGQSPQLLI |
| | | YRVSNRFSGVPDRFSGSGSGTDFTLKISRVEAEDVGVYYCFQGTHVPRTFGQG |
| | | TKVEIKRTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGLSSPVTK |
| | | SFNRGEC |
| DNA Light | 186 | GACATCGTGATGACACAGAGCCCTCTGAGCCTGAGCGTGACCCCTGGACAGCC |
| Chain 2 | | TGCTAGCATCAGCTGCAGAAGCTCTCAGAGCCTGGTGCACAACAACGGCATCA |
| | | CCTACCTGTACTGGTACCTGCAGAAGCCTGGACAGAGCCCTCAGCTGCTGATC |
| | | TACAGAGTGAGCAACAGATTCAGCGGCGTGCCTGACAGATTCTCCGGCTCCGG |
| | | CAGCGGCACCGACTTCACCCTGAAGATCAGCAGAGTGGAGGCCGAGGACGTGG |
| | | GCGTGTACTACTGCTTTCAAGGCACCCATGTCCCTAGAACCTTCGGCCAAGGC |
| | | ACCAAGGTGGAGATCAAGAGAACCGTGGCCGCCCCTAGCGTGTTCATCTTCCC |
| | | TCCTAGCGACGAGCAGCTGAAGAGCGGCACCGCTAGCGTGGTGTGCCTGCTGA |
| | | ACAACTTCTACCCTAGAGAGGCCAAGGTGCAGTGGAAGGTGGACAACGCCCTG |
| | | CAGAGCGGCAACAGCCAAGAGAGCGTGACCGAGCAAGACAGCAAGGACAGCAC |
| | | CTACAGCCTGAGCAGCACCCTGACCCTGAGCAAGGCCGACTACGAGAAGCACA |
| | | AGGTGTACGCCTGCGAGGTGACCCACCAAGGCCTGAGCAGCCCTGTGACCAAG |
| | | AGCTTCAACAGAGGCGAGTGC |
| VL3 | 74 | DIVMTQTPLSSPVTLGQPASISCRSSQSLVHNNGITYLYWYQQRPGQPPRLLI |
| | | YRVSNRFSGVPDRFSGSGAGTDFTLKINRVEAEDVGVYFCFQGTHVPRTFGQG |
| | | TKLEIK |
| DNA VL3 | 163 | GACATCGTGATGACACAGACCCCTCTCAGCAGCCCTGTGACCCTAGGACAGCC |
| | | TGCTAGCATCAGCTGCAGAAGCTCTCAGAGCCTGGTGCACAACAACGGCATCA |
| | | CCTACCTGTACTGGTATCAGCAGAGACCTGGACAGCCTCCTAGACTGCTGATC |
| | | TACAGAGTGAGCAACAGATTCAGCGGAGTACCTGACAGATTTAGCGGTTCCGG |
| | | CGCCGGCACCGACTTCACCCTGAAGATCAACAGAGTGGAGGCCGAGGACGTGG |
| | | GCGTGTACTTCTGCTTTCAAGGCACCCATGTCCCTAGAACCTTCGGCCAAGGC |
| | | ACCAAGCTGGAGATCAAG |
| Light Chain 3 | 177 | DIVMTQTPLSSPVTLGQPASISCRSSQSLVHNNGITYLYWYQQRPGQPPRLLI |
| | | YRVSNRFSGVPDRFSGSGAGTDFTLKINRVEAEDVGVYFCFQGTHVPRTFGQG |
| | | TKLEIKRTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGLSSPVTK |
| | | SFNRGEC |
| DNA Light | 187 | GACATCGTGATGACACAGACCCCTCTCAGCAGCCCTGTGACCCTAGGACAGCC |
| Chain 3 | | TGCTAGCATCAGCTGCAGAAGCTCTCAGAGCCTGGTGCACAACAACGGCATCA |
| | | CCTACCTGTACTGGTATCAGCAGAGACCTGGACAGCCTCCTAGACTGCTGATC |
| | | TACAGAGTGAGCAACAGATTCAGCGGAGTACCTGACAGATTTAGCGGTTCCGG |
| | | CGCCGGCACCGACTTCACCCTGAAGATCAACAGAGTGGAGGCCGAGGACGTGG |
| | | GCGTGTACTTCTGCTTTCAAGGCACCCATGTCCCTAGAACCTTCGGCCAAGGC |
| | | ACCAAGCTGGAGATCAAGAGAACCGTGGCCGCCCCTAGCGTGTTCATCTTCCC |
| | | TCCTAGCGACGAGCAGCTGAAGAGCGGCACCGCTAGCGTGGTGTGCCTGCTGA |
| | | ACAACTTCTACCCTAGAGAGGCCAAGGTGCAGTGGAAGGTGGACAACGCCCTG |
| | | CAGAGCGGCAACAGCCAAGAGAGCGTGACCGAGCAAGACAGCAAGGACAGCAC |
| | | CTACAGCCTGAGCAGCACCCTGACCCTGAGCAAGGCCGACTACGAGAAGCACA |
| | | AGGTGTACGCCTGCGAGGTGACCCACCAAGGCCTGTCAAGCCCTGTAACTAAG |
| | | AGCTTCAACAGAGGCGAGTGC |
| VL4 | 75 | DIEMTQSPLSLPVTLGQPASISCRSSQSLVHNNGITYLYWYQQRPGQSPRRLI |
| | | YRVSNRFSGVPDRFSGSGSGTDFTLKISRVEAEDVGVYFCFQGTHVPRTFGGG |
| | | TKLEIK |
| DNA VL4 | 164 | GACATCGAGATGACACAGAGCCCTCTGAGCCTGCCTGTGACCCTGGGACAGCC |
| | | TGCTAGCATCAGCTGCAGAAGCTCTCAGAGCCTGGTGCACAACAACGGCATCA |
| | | CCTACCTGTACTGGTATCAGCAGAGACCTGGACAGAGCCCTAGAAGACTGATC |
| | | TACAGAGTGAGCAACAGATTCAGCGGCGTGCCTGACAGATTCTCCGGCTCCGG |
| | | CAGCGGCACCGACTTCACCCTGAAGATCAGCAGAGTGGAGGCCGAGGACGTGG |
| | | GCGTGTACTTCTGCTTTCAAGGCACCCATGTCCCTAGAACCTTCGGCGGCGGC |
| | | ACCAAGCTGGAGATCAAG |
| Light Chain 4 | 178 | DIEMTQSPLSLPVTLGQPASISCRSSQSLVHNNGITYLYWYQQRPGQSPRRLI |
| | | YRVSNRFSGVPDRFSGSGSGTDFTLKISRVEAEDVGVYFCFQGTHVPRTFGGG |
| | | TKLEIKRTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGLSSPVTK |
| | | SFNRGEC |
| DNA Light | 188 | GACATCGAGATGACACAGAGCCCTCTGAGCCTGCCTGTGACCCTGGGACAGCC |
| Chain 4 | | TGCTAGCATCAGCTGCAGAAGCTCTCAGAGCCTGGTGCACAACAACGGCATCA |
| | | CCTACCTGTACTGGTATCAGCAGAGACCTGGACAGAGCCCTAGAAGACTGATC |
| | | TACAGAGTGAGCAACAGATTCAGCGGCGTGCCTGACAGATTCTCCGGCTCCGG |
| | | CAGCGGCACCGACTTCACCCTGAAGATCAGCAGAGTGGAGGCCGAGGACGTGG |
| | | GCGTGTACTTCTGCTTTCAAGGCACCCATGTCCCTAGAACCTTCGGCGGCGGC |
| | | ACCAAGCTGGAGATCAAGAGAACCGTGGCCGCCCCTAGCGTGTTCATCTTCCC |
| | | TCCTAGCGACGAGCAGCTGAAGAGCGGCACCGCTAGCGTGGTGTGCCTGCTGA |
| | | ACAACTTCTACCCTAGAGAGGCCAAGGTGCAGTGGAAGGTGGACAACGCCCTG |
| | | CAGAGCGGCAACAGCCAAGAGAGCGTGACCGAGCAAGACAGCAAGGACAGCAC |
| | | CTACAGCCTGAGCAGCACCCTGACCCTGAGCAAGGCCGACTACGAGAAGCACA |
| | | AGGTGTACGCCTGCGAGGTGACCCACCAAGGCCTGAGCAGCCCTGTGACCAAG |
| | | AGCTTCAACAGAGGCGAGTGC |
| VL5 | 76 | EIVLTQSPATLSLSPGERATLSCRSSQSLVHNNGITYLYWYLQKPGQAPKLLI |
| | | YRVSNRFSGIPDRFSGSGPGTDFTLTISSLEPEDLAVYFCFQGTHVPRTFGGG |
| | | TKLEIK |
| DNA VL5 | 165 | GAGATCGTGCTGACACAGAGCCCTGCGACACTGAGCCTGAGCCCTGGCGAGAG |
| | | AGCTACGCTGAGCTGCCGCAGCTCTCAGAGCCTGGTGCACAACAACGGCATCA |
| | | CCTACCTGTACTGGTACCTGCAGAAGCCTGGCCAAGCCCCTAAGCTGCTGATC |
| | | TACAGAGTGAGCAACAGATTCAGCGGCATCCCTGACAGATTCTCCGGCTCCGG |
| | | CCCTGGCACCGACTTCACCCTGACCATCAGCAGCCTGGAGCCTGAGGACCTGG |
| | | CCGTGTACTTCTGCTTCCAAGGCACCCATGTACCTAGAACCTTCGGCGGCGGC |
| | | ACCAAGCTGGAGATCAAG |
| Light Chain 5 | 179 | EIVLTQSPATLSLSPGERATLSCRSSQSLVHNNGITYLYWYLQKPGQAPKLLI |
| | | YRVSNRFSGIPDRFSGSGPGTDFTLTISSLEPEDLAVYFCFQGTHVPRTFGGG |
| | | TKLEIKRTVAAPSVFIFPPSDEQLKSGTASVVCLLNNFYPREAKVQWKVDNAL |
| | | QSGNSQESVTEQDSKDSTYSLSSTLTLSKADYEKHKVYACEVTHQGLSSPVTK |
| | | SFNRGEC |
| DNA Light | 189 | GAGATCGTGCTGACACAGAGCCCTGCGACACTGAGCCTGAGCCCTGGCGAGAG |
| Chain 5 | | AGCTACGCTGAGCTGCCGCAGCTCTCAGAGCCTGGTGCACAACAACGGCATCA |
| | | CCTACCTGTACTGGTACCTGCAGAAGCCTGGCCAAGCCCCTAAGCTGCTGATC |
| | | TACAGAGTGAGCAACAGATTCAGCGGCATCCCTGACAGATTCTCCGGCTCCGG |
| | | CCCTGGCACCGACTTCACCCTGACCATCAGCAGCCTGGAGCCTGAGGACCTGG |
| | | CCGTGTACTTCTGCTTCCAAGGCACCCATGTACCTAGAACCTTCGGCGGCGGC |
| | | ACCAAGCTGGAGATCAAGAGAACCGTGGCCGCCCCTAGCGTGTTCATCTTCCC |
| | | TCCTAGCGACGAGCAGCTGAAGAGCGGCACCGCTAGCGTGGTGTGCCTGCTGA |
| | | ACAACTTCTACCCTAGAGAGGCCAAGGTGCAGTGGAAGGTGGACAACGCCCTG |
| | | CAGAGCGGCAACAGCCAAGAGAGCGTGACCGAGCAAGACAGCAAGGACAGCAC |
| | | CTACAGCCTGAGCAGCACCCTGACCCTGAGCAAGGCCGACTACGAGAAGCACA |
| | | AGGTGTACGCCTGCGAGGTGACCCACCAAGGCCTGAGCAGCCCTGTGACCAAG |
| | | AGCTTCAACAGAGGCGAGTGC |

TABLE 4C
Exemplary anti-tau humanized V0022 antibodies

| | SEQ ID NO | |
|---|---|---|
| Ab ID | or Sequence | Description |
| Ab1 | 314 | HC CDR1 (Chothia) |
| | 341 | HC CDR2 (Chothia) |
| | 410 | HC CDR3 (Chothia) |
| | 1144 | HC CDR1 (Kabat) |
| | 1145 | HC CDR2 (Kabat) |
| | 410 | HC CDR3 (Kabat) |
| | 1165 | HC CDR1 (IMGT) |
| | 1166 | HC CDR2 (IMGT) |
| | 1167 | HC CDR3 (IMGT) |
| | 64 | HC CDR1 (Alternative) |
| | 1145 | HC CDR2 (Alternative) |
| | 1167 | HC CDR3 (Alternative) |
| | 69 | VH3 |
| | 158 | DNA VH3 |
| | 172 | Heavy Chain 3 |
| | 182 | DNA Heavy Chain 3 |
| | 1154 | LC CDR1 (Chothia) |
| | 529 | LC CDR2 (Chothia) |
| | 571 | LC CDR3 (Chothia) |
| | 1146 | LC CDR1 (Kabat) |
| | 529 | LC CDR2 (Kabat) |
| | 571 | LC CDR3 (Kabat) |
| | 473 | LC CDR1 (IMGT) |
| | RVS | LC CDR2 (IMGT) |
| | 571 | LC CDR3 (IMGT) |
| | 1146 | LC CDR1 (Alternative) |
| | 529 | LC CDR2 (Alternative) |
| | 571 | LC CDR3 (Alternative) |
| | 73 | VL2 |
| | 162 | DNA VL2 |
| | 176 | Light Chain 2 |
| | 186 | DNA Light Chain 2 |
| Ab2 | 314 | HC CDR1 (Chothia) |
| | 341 | HC CDR2 (Chothia) |
| | 410 | HC CDR3 (Chothia) |
| | 1144 | HC CDR1 (Kabat) |
| | 1145 | HC CDR2 (Kabat) |
| | 410 | HC CDR3 (Kabat) |
| | 1165 | HC CDR1 (IMGT) |
| | 1166 | HC CDR2 (IMGT) |
| | 1167 | HC CDR3 (IMGT) |
| | 64 | HC CDR1 (Alternative) |
| | 1145 | HC CDR2 (Alternative) |
| | 1167 | HC CDR3 (Alternative) |
| | 67 | VH1 |
| | 156 | DNA VH1 |
| | 170 | Heavy Chain 1 |
| | 180 | DNA Heavy Chain 1 |
| | 1154 | LC CDR1 (Chothia) |
| | 529 | LC CDR2 (Chothia) |
| | 571 | LC CDR3 (Chothia) |
| | 1146 | LC CDR1 (Kabat) |
| | 529 | LC CDR2 (Kabat) |
| | 571 | LC CDR3 (Kabat) |
| | 473 | LC CDR1 (IMGT) |
| | RVS | LC CDR2 (IMGT) |
| | 571 | LC CDR3 (IMGT) |
| | 1146 | LC CDR1 (Alternative) |
| | 529 | LC CDR2 (Alternative) |
| | 571 | LC CDR3 (Alternative) |
| | 72 | VL1 |
| | 161 | DNA VL1 |
| | 175 | Light Chain 1 |
| | 185 | DNA Light Chain 1 |
| Ab3 | 314 | HC CDR1 (Chothia) |
| | 341 | HC CDR2 (Chothia) |
| | 410 | HC CDR3 (Chothia) |
| | 1144 | HC CDR1 (Kabat) |
| | 1145 | HC CDR2 (Kabat) |
| | 410 | HC CDR3 (Kabat) |
| | 1165 | HC CDR1 (IMGT) |
| | 1166 | HC CDR2 (IMGT) |
| | 1167 | HC CDR3 (IMGT) |
| | 64 | HC CDR1 (Alternative) |
| | 1145 | HC CDR2 (Alternative) |
| | 1167 | HC CDR3 (Alternative) |
| | 70 | VH4 |
| | 159 | DNA VH4 |
| | 173 | Heavy Chain 4 |
| | 183 | DNA Heavy Chain 4 |
| | 1154 | LC CDR1 (Chothia) |
| | 529 | LC CDR2 (Chothia) |
| | 571 | LC CDR3 (Chothia) |
| | 1146 | LC CDR1 (Kabat) |
| | 529 | LC CDR2 (Kabat) |
| | 571 | LC CDR3 (Kabat) |
| | 473 | LC CDR1 (IMGT) |
| | RVS | LC CDR2 (IMGT) |
| | 571 | LC CDR3 (IMGT) |
| | 1146 | LC CDR1 (Alternative) |
| | 529 | LC CDR2 (Alternative) |
| | 571 | LC CDR3 (Alternative) |
| | 72 | VL1 |
| | 161 | DNA VL1 |
| | 175 | Light Chain 1 |
| | 185 | DNA Light Chain 1 |
| Ab4 | 314 | HC CDR1 (Chothia) |
| | 341 | HC CDR2 (Chothia) |
| | 410 | HC CDR3 (Chothia) |
| | 1144 | HC CDR1 (Kabat) |
| | 1145 | HC CDR2 (Kabat) |
| | 410 | HC CDR3 (Kabat) |
| | 1165 | HC CDR1 (IMGT) |
| | 1166 | HC CDR2 (IMGT) |
| | 1167 | HC CDR3 (IMGT) |
| | 64 | HC CDR1 (Alternative) |
| | 1145 | HC CDR2 (Alternative) |
| | 1167 | HC CDR3 (Alternative) |
| | 71 | VH5 |
| | 160 | DNA VH5 |
| | 174 | Heavy Chain 5 |
| | 184 | DNA Heavy Chain 5 |
| | 1154 | LC CDR1 (Chothia) |
| | 529 | LC CDR2 (Chothia) |
| | 571 | LC CDR3 (Chothia) |
| | 1146 | LC CDR1 (Kabat) |
| | 529 | LC CDR2 (Kabat) |
| | 571 | LC CDR3 (Kabat) |
| | 473 | LC CDR1 (IMGT) |
| | RVS | LC CDR2 (IMGT) |
| | 571 | LC CDR3 (IMGT) |
| | 1146 | LC CDR1 (Alternative) |
| | 529 | LC CDR2 (Alternative) |
| | 571 | LC CDR3 (Alternative) |
| | 73 | VL2 |
| | 162 | DNA VL2 |
| | 176 | Light Chain 2 |
| | 186 | DNA Light Chain 2 |
| Ab5 | 314 | HC CDR1 (Chothia) |
| | 341 | HC CDR2 (Chothia) |
| | 410 | HC CDR3 (Chothia) |
| | 1144 | HC CDR1 (Kabat) |
| | 1145 | HC CDR2 (Kabat) |
| | 410 | HC CDR3 (Kabat) |
| | 1165 | HC CDR1 (IMGT) |
| | 1166 | HC CDR2 (IMGT) |
| | 1167 | HC CDR3 (IMGT) |
| | 64 | HC CDR1 (Alternative) |
| | 1145 | HC CDR2 (Alternative) |
| | 1167 | HC CDR3 (Alternative) |
| | 71 | VH5 |
| | 160 | DNA VH5 |
| | 174 | Heavy Chain 5 |
| | 184 | DNA Heavy Chain 5 |
| | 1154 | LC CDR1 (Chothia) |
| | 529 | LC CDR2 (Chothia) |
| | 571 | LC CDR3 (Chothia) |
| | 1146 | LC CDR1 (Kabat) |
| | 529 | LC CDR2 (Kabat) |
| | 571 | LC CDR3 (Kabat) |
| | 473 | LC CDR1 (IMGT) |
| | RVS | LC CDR2 (IMGT) |
| | 571 | LC CDR3 (IMGT) |
| | 1146 | LC CDR1 (Alternative) |
| | 529 | LC CDR2 (Alternative) |
| | 571 | LC CDR3 (Alternative) |
| | 74 | VL3 |
| | 163 | DNA VL3 |
| | 177 | Light Chain 3 |
| | 187 | DNA Light Chain 3 |

TABLE 5
Constant Regions of Heavy Chains and Light Chains

| | SEQ | |
|---|---|---|
| Description | ID NO | Sequence |
| Constant | 1189 | AKTTPPSVYPLAPGSAAQTNSMVTLGCLVKGYFPEPVTVTWNSGSLSSGVHTF |
| Region of | | PAVLQSDLYTLSSSVTVPSSPRPSETVTCNVAHPASSTKVDKKIVPRDCGCKP |
| Heavy Chain, | | CICTVPEVSSVFIFPPKPKDVLTITLTPKVTCVVVDISKDDPEVQFSWFVDDV |
| secreted form | | EVHTAQTQPREEQFNSTFRSVSELPIMHQDWLNGKEFKCRVNSAAFPAPIEKT |
| & membrane- | | ISKTKGRPKAPQVYTIPPPKEQMAKDKVSLTCMITDFFPEDITVEWQWNGQPA |
| bound form | | ENYKNTQPIMNINGSYFVYSKLNVQKSNWEAGNTFTCSVLHEGLHNHHTEKSL |
| (mIgG1) | | SHSPGK |
| | 1190 | AKTTPPSVYPLAPGSAAQTNSMVTLGCLVKGYFPEPVTVTWNSGSLSSGVHTF |
| | | PAVLQSDLYTLSSSVTVPSSPRPSETVTCNVAHPASSTKVDKKIVPRDCGCKP |
| | | CICTVPEVSSVFIFPPKPKDVLTITLTPKVTCVVVDISKDDPEVQFSWFVDDV |
| | | EVHTAQTQPREEQFNSTFRSVSELPIMHQDWLNGKEFKCRVNSAAFPAPIEKT |
| | | ISKTKGRPKAPQVYTIPPPKEQMAKDKVSLTCMITDFFPEDITVEWQWNGQPA |
| | | ENYKNTQPIMNINGSYFVYSKLNVQKSNWEAGNTFTCSVLHEGLHNHHTEKSL |
| | | SHSPGLQLDETCAEAQDGELDGLWTTITIFISLFLLSVCYSAAVTLFKVKWIF |
| | | SSVVELKQTLVPEYKNMIGQAP |
| Constant | 1191 | AKTTAPSVYPLAPVCGDTTGSSVTLGCLVKGYFPEPVTLTWNSGSLSSGVHTF |
| Region of | | PAVLQSDLYTLSSSVTVISSTWPSQSITCNVAHPASSTKVDKKIEPRGPTIKP |
| murine Heavy | | CPPCKCPAPNLLGGPSVFIFPPKIKDVLMISLSPIVTCVVVDVSEDDPDVQIS |
| Chain, | | WFVNNVEVHTAQTQTHREDYNSTLRVVSALPIQHQDWMSGKEFKCKVNNKDLP |
| (mIgG2A) | | APIERTISKPKGSVRAPQVYVLPPPEEEMTKKQVTLTCMVTDEMPEDIYVEWT |
| | | NNGKTELNYKNTEPVLDSDGSYFMYSKLRVEKKNWVERNSYSCSVVHEGLHNH |
| | | HTTKSFSRTPGK |
| DNA Constant | 1192 | GCTAAAACAACAGCCCCATCGGTCTATCCACTGGCCCCTGTGTGTGGAGATAC |
| Region of | | AACTGGCTCCTCGGTGACTCTAGGATGCCTGGTCAAGGGTTATTTCCCTGAGC |
| Heavy Chain | | CAGTGACCTTGACCTGGAACTCTGGATCCCTGTCCAGTGGTGTGCACACCTTC |
| (mIgG2A) | | CCAGCTGTCCTGCAGTCTGACCTCTACACCCTCAGCAGCTCAGTGACTGTAAC |
| | | CTCGAGCACCTGGCCCAGCCAGTCCATCACCTGCAATGTGGCCCACCCGGCAA |
| | | GCAGCACCAAGGTGGACAAGAAAATTGAGCCCAGAGGGCCCACAATCAAGCCC |
| | | TGTCCTCCATGCAAATGCCCAGCACCTAACCTCTTGGGTGGACCATCCGTCTT |
| | | CATCTTCCCTCCAAAGATCAAGGATGTACTCATGATCTCCCTGAGCCCCATAG |
| | | TCACATGTGTGGTGGTGGATGTGAGCGAGGATGACCCAGATGTCCAGATCAGC |
| | | TGGTTTGTGAACAACGTGGAAGTACACACAGCTCAGACACAAACCCATAGAGA |
| | | GGATTACAACAGTACTCTCCGGGTGGTCAGTGCCCTCCCCATCCAGCACCAGG |
| | | ACTGGATGAGTGGCAAGGAGTTCAAATGCAAGGTCAACAACAAAGACCTCCCA |
| | | GCGCCCATCGAGAGAACCATCTCAAAACCCAAAGGGTCAGTAAGAGCTCCACA |
| | | GGTATATGTCTTGCCTCCACCAGAAGAAGAGATGACTAAGAAACAGGTCACTC |
| | | TGACCTGCATGGTCACAGACTTCATGCCTGAAGACATTTACGTGGAGTGGACC |
| | | AACAACGGGAAAACAGAGCTAAACTACAAGAACACTGAACCAGTCCTGGACTC |
| | | TGATGGTTCTTACTTCATGTACAGCAAGCTGAGAGTGGAAAAGAAGAACTGGG |
| | | TGGAAAGAAATAGCTACTCCTGTTCAGTGGTCCACGAGGGTCTGCACAATCAC |
| | | CACACGACTAAGAGCTTCTCCCGGACTCCGGGTAAA |
| Constant | 1193 | AKTTAPSVYPLVPVCGGTTGSSVTLGCLVKGYFPEPVTLTWNSGSLSSGVHTF |
| Region of | | PALLQSGLYTLSSSVTVTSNTWPSQTITCNVAHPASSTKVDKKIEPRVPITQN |
| Heavy Chain, | | PCPPHQRVPPCAAPDLLGGPSVFIFPPKIKDVLMISLSPMVTCVVVDVSEDDP |
| secreted form | | DVQISWFVNNVEVHTAQTQTHREDYNSTLRVVSALPIQHQDWMSGKEFKCKVN |
| (mIgG2A) | | NRALPSPIEKTISKPRGPVRAPQVYVLPPPAEEMTKKEFSLTCMITGFLPAEI |
| | | AVDWTSNGRTEQNYKNTATVLDSDGSYFMYSKLRVQKSTWERGSLFACSVVHE |
| | | VLHNHLTTKTISRSLGK |
| Constant | 1194 | KTTPPSVYPLAPGCGDTTGSSVTLGCLVKGYFPESVTVTWNSGSLSSSVHTFP |
| Region of | | ALLQSGLYTMSSSVTVPSSTWPSQTVTCSVAHPASSTTVDKKLEPSGPISTIN |
| murine Heavy | | PCPPCKECHKCPAPNLEGGPSVFIFPPNIKDVLMISLTPKVTCVVVDVSEDDP |
| Chain, | | DVQISWFVNNVEVHTAQTQTHREDYNSTIRVVSTLPIQHQDWMSGKEFKCKVN |
| secreted form | | NKDLPSPIERTISKIKGLVRAPQVYILPPPAEQLSRKDVSLTCLVVGENPGDI |
| (mIgG2B) | | SVEWTSNGHTEENYKDTAPVLDSDGSYFIYSKLNMKTSKWEKTDSFSCNVRHE |
| | | GLKNYYLKKTISRSPGLDLDDICAEAKDGELDGLWTTITIFISLFLLSVCYSA |
| | | SVTLFKVKWIFSSVVELKQKISPDYRNMIGQGA |
| Constant | 1195 | KTTAPSVYPLAPVCGGTTGSSVTLGCLVKGYFPEPVTLTWNSGSLSSGVHTFP |
| Region of | | ALLQSGLYTLSSSVTVTSNTWPSQTITCNVAHPASSTKVDKKIEPRVPITQNP |
| murine Heavy | | CPPLKECPPCAAPDLLGGPSVFIFPPKIKDVLMISLSPMVTCVVVDVSEDDPD |
| Chain, | | VQISWFVNNVEVHTAQTQTHREDYNSTLRVVSALPIQHQDWMSGKEFKCKVNN |
| (mIgG2B) | | RALPSPIEKTISKPRGPVRAPQVYVLPPPAEEMTKKEFSLTCMITGFLPAEIA |
| | | VDWTSNGRTEQNYKNTATVLDSDGSYFMYSKLRVQKSTWERGSLFACSVVHEG |
| | | LHNHLTTKTISRSLGLDLDDVCTEAQDGELDGLWTTITIFISLFLLSVCYSAS |
| | | VTLFKVKWIFSSVVELKQKISPDYRNMIGQGA |
| Constant | 1196 | TTTAPSVYPLVPGCSDTSGSSVTLGCLVKGYFPEPVTVKWNYGALSSGVRTVS |
| Region of | | SVLQSGFYSLSSLVTVPSSTWPSQTVICNVAHPASKTELIKRIEPRIPKPSTP |
| murine Heavy | | PGSSCPPGNILGGPSVFIFPPKPKDALMISLTPKVTCVVVDVSEDDPDVHVSW |
| Chain, | | FVDNKEVHTAWTQPREAQYNSTFRVVSALPIQHQDWMRGKEFKCKVNNKALPA |
| secreted form | | PIERTISKPKGRAQTPQVYTIPPPREQMSKKKVSLTCLVTNFFSEAISVEWER |
| (mIgG3) | | NGELEQDYKNTPPILDSDGTYFLYSKLTVDTDSWLQGEIFTCSVVHEALHNHH |
| | | TQKNLSRSPELELNETCAEAQDGELDGLWTTITIFISLFLLSVCYSASVTLFK |
| | | VKWIFSSVVQVKQTAIPDYRNMIGQGA |
| Constant | 1197 | ADAAPTVSIFPPSSEQLTSGGASVVCFLNNFYPKDINVKWKIDGSERQNGVLN |
| Region of | | SWTDQDSKDSTYSMSSTLTLTKDEYERHNSYTCEATHKTSTSPIVKSENRNEC |
| Murine Kappa | | |
| Light Chain | | |
| DNA Constant | 1198 | GCAGATGCTGCACCAACTGTATCCATCTTCCCACCATCCAGTGAGCAGTTAAC |
| Region of | | ATCTGGAGGTGCCTCAGTCGTGTGCTTCTTGAACAACTTCTACCCCAAAGACA |
| Murine Kappa | | TCAATGTCAAGTGGAAGATTGATGGCAGTGAACGACAAAATGGCGTCCTGAAC |
| Light Chain | | AGTTGGACTGATCAGGACAGCAAAGACAGCACCTACAGCATGAGCAGCACCCT |
| | | CACGTTGACCAAGGACGAGTATGAACGACATAACAGCTATACCTGTGAGGCCA |
| | | CTCACAAGACATCAACTTCACCCATTGTCAAGAGCTTCAACAGGAATGAGTGT |
| Constant | 1199 | QPKSSPSVTLFPPSSEELETNKATLVCTITDFYPGVVTVDWKVDGTPVTQGME |
| Region of | | TTQPSKQSNNKYMASSYLTLTARAWERHSSYSCQVTHEGHTVEKSLSRADCS |
| murine | 1200 | QPKSTPTLTVFPPSSEELKENKATLVCLISNFSPSGVTVAWKANGTPITQGVD |
| Lambda Light | | TSNPTKEGNKFMASSFLHLTSDQWRSHNSFTCQVTHEGDTVEKSLSPAECL |
| Chain | 1201 | QPKSTPTLTMFPPSPEELQENKATLVCLISNFSPSGVTVAWKANGTPITQGVD |
| (subclasses 1, | | TSNPTKEDNKYMASSFLHLTSDQWRSHNSFTCQVTHEGDTVEKSLSPAECL |
| 2, and 3) | | |
| Constant | 1202 | ASTKGPSVFPLAPSSKSTSGGTAALGCLVKDYFPEPVTVSWNSGALTSGVHTF |
| Region of | | PAVLQSSGLYSLSSVVTVPSSSLGTQTYICNVNHKPSNTKVDKRVEPKSCDKT |
| human Heavy | | HTCPPCPAPELLGGPSVFLFPPKPKDTLMISRTPEVTCVVVDVSHEDPEVKEN |
| Chain (hIgG1) | | WYVDGVEVHNAKTKPREEQYNSTYRVVSVLTVLHQDWLNGKEYKCKVSNKALP |
| | | APIEKTISKAKGQPREPQVYTLPPSREEMTKNQVSLTCLVKGFYPSDIAVEWE |
| | | SNGQPENNYKTTPPVLDSDGSFFLYSKLTVDKSRWQQGNVFSCSVMHEALHNH |
| | | YTQKSLSLSPG |
| DNA Constant | 1203 | GCCAGCACAAAAGGGCCCAGTGTGTTCCCGCTCGCACCAAGCAGCAAATCAAC |
| Region of | | GTCAGGCGGCACAGCCGCGTTGGGTTGCCTTGTAAAAGACTACTTCCCAGAAC |
| human Heavy | | CAGTTACGGTGTCATGGAACAGTGGTGCACTCACGAGCGGCGTTCATACCTTC |
| Chain (hIgG1) | | CCCGCCGTACTTCAGAGTTCAGGTCTTTACTCACTTTCCAGCGTGGTCACAGT |
| | | ACCGTCAAGCTCTCTTGGAACACAGACATATATCTGTAACGTAAATCATAAGC |
| | | CTAGCAATACCAAAGTCGATAAACGAGTGGAACCCAAGAGTTGTGATAAAACC |
| | | CACACCTGTCCCCCTTGCCCGGCACCGGAACTGCTGGGTGGTCCATCAGTATT |
| | | CTTGTTTCCGCCTAAGCCAAAGGACACACTGATGATATCCAGAACTCCAGAGG |
| | | TTACGTGCGTAGTCGTGGACGTCAGTCATGAAGACCCCGAAGTTAAGTTCAAC |
| | | TGGTACGTGGATGGTGTGGAAGTACATAATGCGAAGACGAAACCCAGGGAAGA |
| | | ACAATATAACTCAACTTATAGGGTAGTCAGCGTCTTGACTGTACTTCACCAAG |
| | | ATTGGTTGAATGGCAAAGAGTACAAATGCAAGGTAAGCAACAAAGCATTGCCT |
| | | GCGCCAATCGAAAAGACTATCTCAAAAGCAAAGGGCCAGCCACGCGAACCACA |
| | | AGTGTATACATTGCCGCCCAGTCGGGAAGAAATGACGAAAAATCAAGTCAGTC |
| | | TCACATGCCTCGTGAAAGGATTTTATCCCTCTGACATAGCTGTGGAGTGGGAA |
| | | AGTAATGGCCAACCCGAAAATAATTACAAAACGACGCCTCCCGTTTTGGACTC |
| | | AGATGGGAGTTTTTTCCTTTACAGTAAGCTGACGGTTGACAAAAGCAGGTGGC |
| | | AACAAGGGAACGTCTTTTCTTGTAGTGTGATGCATGAGGCGCTCCACAATCAT |
| | | TACACTCAAAAATCCTTGAGCCTGTCTCCAGGC |
| Constant | 1204 | ASTKGPSVFPLAPCSRSTSESTAALGCLVKDYFPEPVTVSWNSGALTSGVHTF |
| Region of | | PAVLQSSGLYSLSSVVTVPSSNFGTQTYTCNVDHKPSNTKVDKTVERKCCVEC |
| Heavy Chain | | PPCPAPPVAGPSVFLFPPKPKDTLMISRTPEVTCVVVDVSHEDPEVQFNWYVD |
| (hIgG2) | | GVEVHNAKTKPREEQFNSTFRVVSVLTVVHQDWLNGKEYKCKVSNKGLPAPIE |
| | | KTISKTKGQPREPQVYTLPPSREEMTKNQVSLTCLVKGFYPSDISVEWESNGQ |
| | | PENNYKTTPPMLDSDGSFFLYSKLTVDKSRWQQGNVFSCSVMHEALHNHYTQK |
| | | SLSLSPGK |
| Constant | 1205 | ASTKGPSVFPLAPCSRSTSGGTAALGCLVKDYFPEPVTVSWNSGALTSGVHTF |
| Region of | | PAVLQSSGLYSLSSVVTVPSSSLGTQTYTCNVNHKPSNTKVDKRVELKTPLGD |
| Heavy Chain | | TTHTCPRCPEPKSCDTPPPCPRCPEPKSCDTPPPCPRCPEPKSCDTPPPCPRC |
| (hIgG3) | | PAPELLGGPSVFLFPPKPKDTLMISRTPEVTCVVVDVSHEDPEVQFKWYVDGV |
| | | EVHNAKTKPREEQYNSTFRVVSVLTVLHQDWLNGKEYKCKVSNKALPAPIEKT |
| | | ISKTKGQPREPQVYTLPPSREEMTKNQVSLTCLVKGFYPSDIAVEWESSGQPE |
| | | NNYNTTPPMLDSDGSFFLYSKLTVDKSRWQQGNIFSCSVMHEALHNRFTQKSL |
| | | SLSPGK |
| Constant | 1206 | ASTKGPSVFPLAPCSRSTSESTAALGCLVKDYFPEPVTVSWNSGALTSGVHTF |
| Region of | | PAVLQSSGLYSLSSVVTVPSSSLGTKTYTCNVDHKPSNTKVDKRVESKYGPPC |
| human Heavy | | PSCPAPEFLGGPSVFLFPPKPKDTLMISRTPEVTCVVVDVSQEDPEVQFNWYV |
| Chain (hIgG4) | | DGVEVHNAKTKPREEQFNSTYRVVSVLTVLHQDWLNGKEYKCKVSNKGLPSSI |
| | | EKTISKAKGQPREPQVYTLPPSQEEMTKNQVSLTCLVKGFYPSDIAVEWESNG |
| | | QPENNYKTTPPVLDSDGSFFLYSRLTVDKSRWQEGNVFSCSVMHEALHNHYTQ |
| | | KSLSLSLGK |
| Constant | 1207 | GQPKANPTVTLFPPSSEELQANKATLVCLISDFYPGAVTVAWKADGSPVKAGV |
| Region of | | ETTKPSKQSNNKYAASSYLSLTPEQWKSHRSYSCQVTHEGSTVEKTVAPTECS |
| human Light | 1208 | GQPKAAPSVTLFPPSSEELQANKATLVCLISDFYPGAVTVAWKADSSPVKAGV |
| Chain (hIgG | | ETTTPSKQSNNKYAASSYLSLTPEQWKSHRSYSCQVTHEGSTVEKTVAPTECS |
| Lambda, | 1209 | GQPKAAPSVTLFPPSSEELQANKATLVCLISDFYPGAVTVAWKADSSPVKAGV |
| subclasses 1, | | ETTTPSKQSNNKYAASSYLSLTPEQWKSHKSYSCQVTHEGSTVEKTVAPTECS |
| 2, 3, 6, | 1210 | GQPKAAPSVTLFPPSSEELQANKATLVCLISDFYPGAVKVAWKADGSPVNTGV |
| and 7) | | ETTTPSKQSNNKYAASSYLSLTPEQWKSHRSYSCQVTHEGSTVEKTVAPAECS |
| | 1211 | GQPKAAPSVTLFPPSSEELQANKATLVCLVSDFNPGAVTVAWKADGSPVKVGV |
| | | ETTKPSKQSNNKYAASSYLSLTPEQWKSHRSYSCRVTHEGSTVEKTVAPAECS |
| Constant | 1212 | TVAAPSVFIFPPSDEQLKSGTASVVCLLNNFYPREAKVQWKVDNALQSGNSQE |
| Region of | | SVTEQDSKDSTYSLSSTLTLSKADYEKHKVYACEVTHQGLSSPVTKSENRGEC |
| human Light | | |
| Chain (hIgG | | |
| Kappa) | | |
| DNA Constant | 1213 | ACTGTCGCAGCACCTTCTGTCTTCATCTTTCCGCCAAGCGATGAACAGTTGAA |
| Region of | | ATCTGGAACAGCGTCCGTGGTGTGCCTGCTCAACAACTTCTATCCTCGGGAAG |
| human Light | | CGAAGGTGCAATGGAAGGTAGATAATGCTCTTCAGAGTGGCAATTCCCAAGAG |
| Chain (hIgG | | TCAGTTACGGAGCAAGATAGCAAGGACAGCACGTATTCCCTGTCTAGTACGTT |
| kappa) | | GACTCTTTCCAAGGCTGACTATGAAAAGCACAAGGTGTATGCCTGTGAAGTAA |
| | | CCCACCAAGGTCTCTCAAGTCCTGTAACTAAGAGCTTTAATCGAGGAGAATGC |
| Murine IgG1 | 805 | GCCAAAACGACACCCCCATCTGTCTATCCACTGGCCCCTGGATCTGCTGCCCA |
| Heavy Chain | | AACTAACTCCATGGTGACCCTGGGATGCCTGGTCAAGGGCTATTTCCCTGAGC |
| constant | | CAGTGACAGTGACCTGGAACTCTGGATCCCTGTCCAGCGGTGTGCACACCTTC |
| region (DNA) | | CCAGCTGTCCTGCAGTCTGACCTCTACACTCTGAGCAGCTCAGTGACTGTCCC |
| | | CTCCAGCACCTGGCCCAGCGAGACCGTCACCTGCAACGTTGCCCACCCGGCCA |
| | | GCAGCACCAAGGTGGACAAGAAAATTGTGCCCAGGGATTGTGGTTGTAAGCCT |
| | | TGCATATGTACAGTCCCAGAAGTATCATCTGTCTTCATCTTCCCCCCAAAGCC |
| | | CAAGGATGTGCTCACCATTACTCTGACTCCTAAGGTCACGTGTGTTGTGGTAG |
| | | ACATCAGCAAGGATGATCCCGAGGTCCAGITCAGCTGGTTTGTAGATGATGTG |
| | | GAGGTGCACACAGCTCAGACGCAACCCCGGGAGGAGCAGTTCAACAGCACTTT |
| | | CCGCTCAGTCAGTGAACTTCCCATCATGCACCAGGACTGGCTCAATGGCAAGG |
| | | AGTTCAAATGCAGGGTCAACAGTGCAGCTTTCCCTGCCCCCATCGAGAAAACC |
| | | ATCTCCAAAACCAAAGGCAGACCGAAGGCTCCACAGGTGTACACCATTCCACC |
| | | TCCCAAGGAGCAGATGGCCAAGGATAAAGTCAGTCTGACCTGCATGATAACAG |
| | | ACTTCTTCCCTGAAGACATTACTGTGGAGTGGCAGTGGAATGGGCAGCCAGCG |
| | | GAGAACTACAAGAACACTCAGCCCATCATGGACACAGATGGCTCTTACTTCGT |
| | | CTACAGCAAGCTCAATGTGCAGAAGAGCAACTGGGAGGCAGGAAATACTTTCA |
| | | CCTGCTCTGTGTTACATGAGGGCCTGCACAACCACCATACTGAGAAGAGCCTC |
| | | TCCCACTCTCCTGGT |
| Murine IgG1 | 16 | AKTTPPSVYPLAPGSAAQTNSMVTLGCLVKGYFPEPVTVTWNSGSLSSGVHTF |
| Heavy Chain | | PAVLQSDLYTLSSSVTVPSSTWPSETVTCNVAHPASSTKVDKKIVPRDCGCKP |
| constant | | CICTVPEVSSVFIFPPKPKDVLTITLTPKVTCVVVDISKDDPEVQFSWFVDDV |
| region (amino | | EVHTAQTQPREEQFNSTFRSVSELPIMHQDWLNGKEFKCRVNSAAFPAPIEKT |
| acid) | | ISKTKGRPKAPQVYTIPPPKEQMAKDKVSLTCMITDFFPEDITVEWQWNGQPA |
| | | ENYKNTQPIMDTDGSYFVYSKLNVQKSNWEAGNTFTCSVLHEGLHNHHTEKSL |
| | | SHSPG |
| Murine IgG1 | 17 | CGGGCTGATGCTGCACCAACTGTATCCATCTTCCCACCATCCAGTGAGCAGTT |
| kappa light | | AACATCTGGAGGTGCCTCAGTCGTGTGCTTCTTGAACAACTTCTACCCCAAAG |
| chain constant | | ACATCAATGTCAAGTGGAAGATTGATGGCAGTGAACGACAAAATGGCGTCCTG |
| region (DNA) | | AACAGTTGGACTGATCAGGACAGCAAAGACAGCACCTACAGCATGAGCAGCAC |
| | | CCTCACGTTGACCAAGGACGAGTATGAACGACATAACAGCTATACCTGTGAGG |
| | | CCACTCACAAGACATCAACTTCACCCATTGTCAAGAGCTTCAACAGGAATGAG |
| | | TGT |
| Murine IgG1 | 18 | RADAAPTVSIFPPSSEQLTSGGASVVCFLNNFYPKDINVKWKIDGSERQNGVL |
| kappa light | | NSWTDQDSKDSTYSMSSTLTLTKDEYERHNSYTCEATHKTSTSPIVKSENRNE |
| chain constant | | C |
| region (amino | | |
| acid) | | |
| Human heavy | 194 | ASTKGPSVFPLAPCSRSTSESTAALGCLVKDYFPEPVTVSWNSGALTSGVHTF |
| Chain | | PAVLQSSGLYSLSSVVTVPSSSLGTKTYTCNVDHKPSNTKVDKRVESKYGPPC |
| Constant | | PPCPAPEFLGGPSVFLFPPKPKDTLMISRTPEVTCVVVDVSQEDPEVQFNWYV |
| Region | | DGVEVHNAKTKPREEQFNSTYRVVSVLTVLHQDWLNGKEYKCKVSNKGLPSSI |
| (Human IgG4) | | EKTISKAKGQPREPQVYTLPPSQEEMTKNQVSLTCLVKGFYPSDIAVEWESNG |
| | | QPENNYKTTPPVLDSDGSFFLYSRLTVDKSRWQEGNVFSCSVMHEALHNHYTQ |
| | | KSLSLSLGK |
| DNA Human | 195 | GCTAGCACCAAGGGTCCTTCCGTGTTTCCTCTAGCCCCTTGCAGCAGAAGCAC |
| heavy Chain | | AAGCGAGAGCACCGCCGCCCTGGGCTGCCTGGTTAAAGATTATTTCCCTGAGC |
| Constant | | CTGTGACCGTGAGCTGGAATAGCGGCGCCCTGACAAGCGGCGTGCACACCTTC |
| Region | | CCTGCCGTGCTGCAGAGCAGCGGCCTGTACAGCCTGAGCAGCGTGGTGACCGT |
| (Human IgG4) | | GCCTTCTAGCAGCCTGGGTACTAAGACCTACACCTGCAACGTGGACCACAAGC |
| | | CTAGCAACACCAAGGTGGACAAGAGAGTGGAGAGCAAGTACGGTCCTCCGTGT |
| | | CCCCCGTGCCCTGCCCCTGAGTTCCTGGGCGGCCCTAGCGTTTTCTTGTTTCC |
| | | ACCTAAGCCTAAGGACACCCTGATGATCAGCAGAACCCCTGAGGTGACCTGCG |
| | | TGGTGGTGGACGTGAGCCAAGAGGACCCTGAGGTGCAGTTCAACTGGTACGTG |
| | | GACGGCGTGGAGGTGCACAACGCCAAGACCAAGCCTAGAGAGGAGCAGTTCAA |
| | | CAGCACCTACAGAGTGGTGAGCGTGCTGACCGTGCTGCACCAAGACTGGCTGA |
| | | ACGGCAAGGAGTACAAGTGCAAGGTGAGCAACAAGGGCCTGCCTAGCAGTATT |
| | | GAAAAGACCATCAGCAAGGCCAAGGGACAGCCTAGAGAGCCTCAAGTGTACAC |
| | | CCTGCCTCCTAGCCAAGAGGAGATGACCAAGAACCAAGTGAGCCTGACCTGCC |
| | | TGGTGAAGGGGTTTTACCCTAGCGACATCGCCGTGGAGTGGGAGAGCAACGGA |
| | | CAGCCTGAGAACAACTACAAGACCACCCCTCCTGTGCTGGACAGCGACGGCAG |
| | | CTTCTTCCTGTACAGCAGACTGACCGTGGACAAGAGCAGATGGCAAGAGGGCA |
| | | ACGTGTTCAGCTGCAGCGTGATGCACGAGGCCCTGCACAACCACTACACACAG |
| | | AAGAGCCTGAGCCTGAGCCTGGGCAAG |
| DNA Human | 196 | GCTAGCACGAAAGGTCCAAGCGTTTTCCCTCTAGCCCCTTGCAGCAGAAGCAC |
| heavy Chain | | AAGCGAGAGCACCGCCGCCCTGGGCTGCCTTGTAAAAGATTACTTCCCTGAGC |
| Constant | | CTGTGACCGTGAGTTGGAACAGCGGCGCCCTGACAAGCGGCGTGCACACCTTC |
| Region | | CCTGCCGTGCTGCAGAGCAGCGGCCTGTACAGCCTGAGCAGCGTGGTGACCGT |
| (Human IgG4) | | GCCAAGCAGCAGCCTGGGCACCAAGACCTACACCTGCAACGTGGACCACAAGC |
| | | CTAGCAACACCAAGGTGGACAAGAGAGTGGAGAGCAAGTACGGTCCTCCGTGT |
| | | CCCCCGTGCCCTGCCCCTGAGTTCCTGGGCGGCCCTTCGGTGTTTCTGTTTCC |
| | | ACCTAAGCCTAAGGACACCCTGATGATCAGCAGAACCCCTGAGGTGACCTGCG |
| | | TGGTGGTGGACGTGAGCCAAGAGGACCCTGAGGTGCAGTTCAACTGGTACGTG |
| | | GACGGCGTGGAGGTGCACAACGCCAAGACCAAGCCTAGAGAGGAGCAGTTCAA |
| | | CAGCACCTACAGAGTGGTGAGCGTGCTGACCGTGCTGCACCAAGACTGGCTGA |
| | | ACGGCAAGGAGTACAAGTGCAAGGTGAGCAACAAGGGCCTGCCTAGTTCCATT |
| | | GAGAAGACCATCAGCAAGGCCAAGGGACAGCCTAGAGAGCCTCAAGTGTACAC |
| | | CCTGCCTCCTAGCCAAGAGGAGATGACCAAGAACCAAGTGAGCCTGACCTGCC |
| | | TGGTGAAGGGATTCTACCCTAGCGACATCGCCGTGGAGTGGGAGAGCAACGGA |
| | | CAGCCTGAGAACAACTACAAGACCACCCCTCCTGTGCTGGACAGCGACGGCAG |
| | | CTTCTTCCTGTACAGCAGACTGACCGTGGACAAGAGCAGATGGCAAGAGGGCA |
| | | ACGTGTTCAGCTGCAGCGTGATGCACGAGGCCCTGCACAACCACTACACACAG |
| | | AAGAGCCTGAGCCTGAGCCTGGGCAAG |
| DNA Human | 198 | GCTAGCACCAAGGGTCCTAGTGTATTTCCCCTAGCCCCTTGCAGCAGAAGCAC |
| heavy Chain | | AAGCGAGAGCACCGCCGCCCTGGGCTGCTTGGTGAAGGACTACTTCCCTGAGC |
| Constant | | CTGTCACAGTGTCCTGGAATAGCGGCGCCCTGACAAGCGGCGTGCACACCTTC |
| Region | | CCTGCCGTGCTGCAGAGCAGCGGCCTGTACAGCCTGAGCAGCGTGGTGACCGT |
| (Human IgG4) | | GCCTTCGTCGAGCCTGGGCACCAAGACCTACACCTGCAACGTGGACCACAAGC |
| | | CTAGCAACACCAAGGTGGACAAGAGAGTGGAGAGCAAGTACGGTCCTCCGTGT |
| | | CCCCCGTGCCCTGCCCCTGAGTTCCTGGGCGGCCCTAGCGTGTTCCTATTTCC |
| | | ACCTAAGCCTAAGGACACCCTGATGATCAGCAGAACCCCTGAGGTGACCTGCG |
| | | TGGTGGTGGACGTGAGCCAAGAGGACCCTGAGGTGCAGTTCAACTGGTACGTG |
| | | GACGGCGTGGAGGTGCACAACGCCAAGACCAAGCCTAGAGAGGAGCAGTTCAA |
| | | CAGCACCIACAGAGTGGTGAGCGTGCTGACCGTGCTGCACCAAGACTGGCTGA |
| | | ACGGCAAGGAGTACAAGTGCAAGGTGAGCAACAAGGGCCTGCCTAGCAGTATC |
| | | GAGAAGACCATCAGCAAGGCCAAGGGACAGCCTAGAGAGCCTCAAGTGTACAC |
| | | CCTGCCTCCTAGCCAAGAGGAGATGACCAAGAACCAAGTGAGCCTGACCTGCC |
| | | TGGTAAAAGGTTTCTACCCTAGCGACATCGCCGTGGAGTGGGAGAGCAACGGA |
| | | CAGCCTGAGAACAACTACAAGACCACCCCTCCTGTGCTGGACAGCGACGGCAG |
| | | CTTCTTCCTGTACAGCAGACTGACCGTGGACAAGAGCAGATGGCAAGAGGGCA |
| | | ACGTGTTCAGCTGCAGCGTGATGCACGAGGCCCTGCACAACCACTACACACAG |
| | | AAGAGCCTGAGCCTGAGCCTGGGCAAG |
| DNA Human | 199 | GCTAGCACCAAGGGTCCTAGTGTATTTCCCCTAGCCCCTTGCAGCAGAAGCAC |
| heavy Chain | | AAGCGAGAGCACCGCCGCCCTGGGCTGCTTGGTGAAGGACTACTTCCCTGAGC |
| Constant | | CTGTGACCGTGAGCTGGAACAGCGGCGCCCTGACAAGCGGCGTGCACACCTTC |
| Region | | CCTGCCGTGCTGCAGAGCAGCGGCCTGTACAGCCTGAGCAGCGTGGTGACCGT |
| (Human IgG4) | | GCCTTCGTCGAGCCTGGGCACCAAGACCTACACCTGCAACGTGGACCACAAGC |
| | | CTAGCAACACCAAGGTGGACAAGAGAGTGGAGAGCAAGTACGGTCCTCCGTGT |
| | | CCCCCGTGCCCTGCCCCTGAGTTCCTGGGCGGCCCTAGCGTGTTCCTATTTCC |
| | | ACCTAAGCCTAAGGACACCCTGATGATCAGCAGAACCCCTGAGGTGACCTGCG |
| | | TGGTGGTGGACGTGAGCCAAGAGGACCCTGAGGTGCAGTTCAACTGGTACGTG |
| | | GACGGCGTGGAGGTGCACAACGCCAAGACCAAGCCTAGAGAGGAGCAGTTCAA |
| | | CAGCACCTACAGAGTGGTGAGCGTGCTGACCGTGCTGCACCAAGACTGGCTGA |
| | | ACGGCAAGGAGTACAAGTGCAAGGTGAGCAACAAGGGCCTGCCTAGCAGTATC |
| | | GAGAAGACCATCAGCAAGGCCAAGGGACAGCCTAGAGAGCCTCAAGTGTACAC |
| | | CCTGCCTCCTAGCCAAGAGGAGATGACCAAGAACCAAGTGAGCCTGACCTGCC |
| | | TGGTAAAAGGTTTCTACCCTAGCGACATCGCCGTGGAGTGGGAGAGCAACGGA |
| | | CAGCCTGAGAACAACTACAAGACCACCCCTCCTGTGCTGGACAGCGACGGCAG |
| | | CTTCTTCCTGTACTCCCGCCTGACGGTTGACAAGAGCAGATGGCAAGAGGGCA |
| | | ACGTGTTCAGCTGCAGCGTGATGCACGAGGCCCTGCACAACCACTACACACAG |
| | | AAGAGCCTGAGCCTGAGCCTGGGCAAG |
| Human light | 200 | RTVAAPSVFIFPPSDEQLKSGTASVVCLLNNFYPREAKVQWKVDNALQSGNSQ |
| Chain | | ESVTEQDSKDSTYSLSSTLTLSKADYEKHKVYACEVTHQGLSSPVTKSENRGE |
| Constant | | C |
| region | | |
| (Human IgG | | |
| kappa) | | |
| DNA Human | 201 | AGAACCGTGGCCGCCCCTAGCGTGTTCATCTTCCCTCCTAGCGACGAGCAGCT |
| light chain | | GAAGAGCGGCACCGCTAGCGTGGTGTGCCTGCTGAACAACTTCTACCCTAGAG |
| Constant | | AGGCCAAGGTGCAGTGGAAGGTGGACAACGCCCTGCAGAGCGGCAACAGCCAA |
| region | | GAGAGCGTGACCGAGCAAGACAGCAAGGACAGCACCTACAGCCTGAGCAGCAC |
| (Human IgG | | CCTGACCCTGAGCAAGGCCGACTACGAGAAGCACAAGGTGTACGCCTGCGAGG |
| kappa) | | TGACCCACCAAGGCCTGAGCAGCCCTGTGACCAAGAGCTTCAACAGAGGCGAG |
| | | TGC |
| DNA Human | 202 | AGAACCGTGGCCGCCCCTAGCGTGTTCATCTTCCCTCCTAGCGACGAGCAGCT |
| light Chain | | GAAGAGCGGCACCGCTAGCGTGGTGTGCCTGCTGAACAACTTCTACCCTAGAG |
| Constant | | AGGCCAAGGTGCAGTGGAAGGTGGACAACGCCCTGCAGAGCGGCAACAGCCAA |
| region | | GAGAGCGTGACCGAGCAAGACAGCAAGGACAGCACCTACAGCCTGAGCAGCAC |
| (Human IgG | | CCTGACCCTGAGCAAGGCCGACTACGAGAAGCACAAGGTGTACGCCTGCGAGG |
| kappa) | | TGACCCACCAAGGCCTGTCAAGCCCTGTAACTAAGAGCTTCAACAGAGGCGAG |
| | | TGC |

In some embodiments, the anti-tau antibody comprises at least one, two, three, or all of a heavy chain complementary determining region 1 (HC CDR1), a HC CDR2, and/or a HC CDR3, wherein the HC CDR1, HC CDR2, and HC CDR3 sequences comprise the sequences of SEQ ID NO: 299, 343, and 395, respectively. In some embodiments, the anti-tau antibody comprises at least one, two, three, or all of a LC CDR1, a LC CDR2 and/or an LC CDR3, wherein the LC CDR1, LC CDR2, and LC CDR3 sequences comprise the sequences of SEQ ID NO: 460, 518, and 557, respectively. In some embodiments, the anti-tau antibody comprises a HC CDR1, a HC CDR2, a HC CDR3, a LC CDR1, a LC CDR2 and an LC CDR3, wherein the HC CDR1, HC CDR2, HC CDR3, LC CDR1, LC CDR2, and LC CDR3 sequences comprise the sequences of SEQ ID NO: 299, 343, 395, 460, 518, and 557, respectively. In some embodiments, one or more of the CDRs (or collectively all of the CDRs) have one, two, three, four, five or more changes, e.g., amino acid substitutions, insertions, or deletions, relative to the amino acid sequence of any of SEQ ID NO: 299, 343, 395, 460, 518, or 557.

In some embodiments, the anti-tau antibody comprises at least one, two, three, or all of a heavy chain complementary determining region 1 (HC CDR1), a HC CDR2, and/or a HC CDR3, wherein the HC CDR1, HC CDR2, and HC CDR3 sequences comprise the sequences of SEQ ID NO: 1140, 1141, and 395, respectively. In some embodiments, the anti-tau antibody comprises at least one, two, three, or all of a LC CDR1, a LC CDR2 and/or an LC CDR3, wherein the LC CDR1, LC CDR2, and LC CDR3 sequences comprise the sequences of SEQ ID NO: 460, 518, and 557, respectively. In some embodiments, the anti-tau antibody comprises a HC CDR1, a HC CDR2, a HC CDR3, a LC CDR1, a LC CDR2 and an LC CDR3, wherein the HC CDR1, HC CDR2, HC CDR3, LC CDR1, LC CDR2, and LC CDR3 sequences comprise the sequences of SEQ ID NO: 1140, 1141, 395, 460, 518, and 557, respectively. In some embodiments, one or more of the CDRs (or collectively all of the CDRs) have one, two, three, four, five or more changes, e.g., amino acid substitutions, insertions, or deletions, relative to the amino acid sequence of any of SEQ ID NO: 1140, 1141, 395, 460, 518, and 557.

In some embodiments, the anti-tau antibody comprises at least one, two, three, or all of a heavy chain complementary determining region 1 (HC CDR1), a HC CDR2, and/or a HC CDR3, wherein the HC CDR1, HC CDR2, and HC CDR3 sequences comprise the sequences of SEQ ID NO: 1155, 1156, and 1157, respectively. In some embodiments, the anti-tau antibody comprises at least one, two, three, or all of a LC CDR1, a LC CDR2 and/or an LC CDR3, wherein the LC CDR1, LC CDR2, and LC CDR3 sequences comprise the sequences of SEQ ID NO: 1158, NAK, and SEQ ID NO: 557, respectively. In some embodiments, the anti-tau antibody comprises a HC CDR1, a HC CDR2, a HC CDR3, a LC CDR1, a LC CDR2 and an LC CDR3, wherein the HC CDR1, HC CDR2, HC CDR3, LC CDR1, LC CDR2, and LC CDR3 sequences comprise the sequences of SEQ ID NO: 1155, SEQ ID NO: 1156, SEQ ID NO: 1157, SEQ ID NO: 1158, NAK, and SEQ ID NO: 557, respectively. In some embodiments, one or more of the CDRs (or collectively all of the CDRs) have one, two, three, four, five or more changes, e.g., amino acid substitutions, insertions, or deletions, relative to the amino acid sequence of any of SEQ ID NO: 1155, SEQ ID NO: 1156, SEQ ID NO: 1157, SEQ ID NO: 1158, NAK, and SEQ ID NO: 557.

In some embodiments, the anti-tau antibody comprises at least one, two, three, or all of a heavy chain complementary determining region 1 (HC CDR1), a HC CDR2, and/or a HC CDR3, wherein the HC CDR1, HC CDR2, and HC CDR3 sequences comprise the sequences of SEQ ID NO: 304, 347, and 400, respectively. In some embodiments, the anti-tau antibody comprises at least one, two, three, or all of a LC CDR1, a LC CDR2 and/or an LC CDR3, wherein the LC CDR1, LC CDR2, and LC CDR3 sequences comprise the sequences of SEQ ID NO: 464, 523, and 562, respectively. In some embodiments, the anti-tau antibody comprises a HC CDR1, a HC CDR2, a HC CDR3, a LC CDR1, a LC CDR2 and an LC CDR3, wherein the HC CDR1, HC CDR2, HC CDR3, LC CDR1, LC CDR2, and LC CDR3 sequences comprise the sequences of SEQ ID NO: 304, 347, 400, 464, 523, and 562, respectively. In some embodiments, one or more of the CDRs (or collectively all of the CDRs) have one, two, three, four, five or more changes, e.g., amino acid substitutions, insertions, or deletions, relative to the amino acid sequence of any of SEQ ID NO: 304, 347, 400, 464, 523, and 562.

In some embodiments, the anti-tau antibody comprises at least one, two, three, or all of a heavy chain complementary determining region 1 (HC CDR1), a HC CDR2, and/or a HC CDR3, wherein the HC CDR1, HC CDR2, and HC CDR3 sequences comprise the sequences of SEQ ID NO: 1142, 1143, and 400, respectively. In some embodiments, the anti-tau antibody comprises at least one, two, three, or all of a LC CDR1, a LC CDR2 and/or an LC CDR3, wherein the LC CDR1, LC CDR2, and LC CDR3 sequences comprise the sequences of SEQ ID NO: 464, 523, and 562, respectively. In some embodiments, the anti-tau antibody comprises a HC CDR1, a HC CDR2, a HC CDR3, a LC CDR1, a LC CDR2 and an LC CDR3, wherein the HC CDR1, HC CDR2, HC CDR3, LC CDR1, LC CDR2, and LC CDR3 sequences comprise the sequences of SEQ ID NO: 1142, 1143, 400, 464, 523, and 562, respectively. In some embodiments, one or more of the CDRs (or collectively all of the CDRs) have one, two, three, four, five or more changes, e.g., amino acid substitutions, insertions, or deletions, relative to the amino acid sequence of any of SEQ ID NO: 1142, 1143, 400, 464, 523, and 562.

In some embodiments, the anti-tau antibody comprises at least one, two, three, or all of a heavy chain complementary determining region 1 (HC CDR1), a HC CDR2, and/or a HC CDR3, wherein the HC CDR1, HC CDR2, and HC CDR3 sequences comprise the sequences of SEQ ID NO: 1160, 1161, and 1162, respectively. In some embodiments, the anti-tau antibody comprises at least one, two, three, or all of a LC CDR1, a LC CDR2 and/or an LC CDR3, wherein the LC CDR1, LC CDR2, and LC CDR3 sequences comprise the sequences of SEQ ID NO: 1163, WAS, and SEQ ID NO: 562, respectively. In some embodiments, the anti-tau antibody comprises a HC CDR1, a HC CDR2, a HC CDR3, a LC CDR1, a LC CDR2 and an LC CDR3, wherein the HC CDR1, HC CDR2, HC CDR3, LC CDR1, LC CDR2, and LC CDR3 sequences comprise the sequences of SEQ ID NO: 1160, SEQ ID NO: 1161, SEQ ID NO: 1162, SEQ ID NO: 1163, WAS, and SEQ ID NO: 562, respectively. In some embodiments, one or more of the CDRs (or collectively all of the CDRs) have one, two, three, four, five or more changes, e.g., amino acid substitutions, insertions, or deletions, relative to the amino acid sequence of any of SEQ ID NO: 1160, SEQ ID NO: 1161, SEQ ID NO: 1162, SEQ ID NO: 1163, WAS, and SEQ ID NO: 562.

In some embodiments, the anti-tau antibody comprises at least one, two, three, or all of a heavy chain complementary determining region 1 (HC CDR1), a HC CDR2, and/or a HC CDR3, wherein the HC CDR1, HC CDR2, and HC CDR3 sequences comprise the sequences of SEQ ID NO: 314, 341, and 410, respectively. In some embodiments, the anti-tau antibody comprises at least one, two, three, or all of a LC CDR1, a LC CDR2 and/or an LC CDR3, wherein the LC CDR1, LC CDR2, and LC CDR3 sequences comprise the sequences of SEQ ID NO: 1154, 529, and 571, respectively. In some embodiments, the anti-tau antibody comprises a HC CDR1, a HC CDR2, a HC CDR3, a LC CDR1, a LC CDR2 and an LC CDR3, wherein the HC CDR1, HC CDR2, HC CDR3, LC CDR1, LC CDR2, and LC CDR3 sequences comprise the sequences of SEQ ID NO: 314, 341, 410, 1154, 529, and 571, respectively. In some embodiments, one or more of the CDRs (or collectively all of the CDRs) have one, two, three, four, five or more changes, e.g., amino acid substitutions, insertions, or deletions, relative to the amino acid sequence of any of SEQ ID NO: 314, 341, 410, 1154, 529, and 571.

In some embodiments, the anti-tau antibody comprises at least one, two, three, or all of a heavy chain complementary determining region 1 (HC CDR1), a HC CDR2, and/or a HC CDR3, wherein the HC CDR1, HC CDR2, and HC CDR3 sequences comprise the sequences of SEQ ID NO: 1144, 1145, and 410, respectively. In some embodiments, the anti-tau antibody comprises at least one, two, three, or all of a LC CDR1, a LC CDR2 and/or an LC CDR3, wherein the LC CDR1, LC CDR2, and LC CDR3 sequences comprise the sequences of SEQ ID NO: 1146, 529, and 571, respectively. In some embodiments, the anti-tau antibody comprises a HC CDR1, a HC CDR2, a HC CDR3, a LC CDR1, a LC CDR2 and an LC CDR3, wherein the HC CDR1, HC CDR2, HC CDR3, LC CDR1, LC CDR2, and LC CDR3 sequences comprise the sequences of SEQ ID NO: 1144, 1145, 410, 1146, 529, and 571, respectively. In some embodiments, one or more of the CDRs (or collectively all of the CDRs) have one, two, three, four, five or more changes, e.g., amino acid substitutions, insertions, or deletions, relative to the amino acid sequence of any of SEQ ID NO: 1144, 1145, 410, 1146, 529, and 571.

In some embodiments, the anti-tau antibody comprises at least one, two, three, or all of a heavy chain complementary determining region 1 (HC CDR1), a HC CDR2, and/or a HC CDR3, wherein the HC CDR1, HC CDR2, and HC CDR3 sequences comprise the sequences of SEQ ID NO: 1165, 1166, and 1167, respectively. In some embodiments, the anti-tau antibody comprises at least one, two, three, or all of a LC CDR1, a LC CDR2 and/or an LC CDR3, wherein the LC CDR1, LC CDR2, and LC CDR3 sequences comprise the sequences of SEQ ID NO: 473, RVS, and SEQ ID NO: 571, respectively. In some embodiments, the anti-tau antibody comprises a HC CDR1, a HC CDR2, a HC CDR3, a LC CDR1, a LC CDR2 and an LC CDR3, wherein the HC CDR1, HC CDR2, HC CDR3, LC CDR1, LC CDR2, and LC CDR3 sequences comprise the sequences of SEQ ID NO: 1165, SEQ ID NO: 1166, SEQ ID NO: 1167, SEQ ID NO: 473, RVS, and SEQ ID NO: 571, respectively. In some embodiments, one or more of the CDRs (or collectively all of the CDRs) have one, two, three, four, five or more changes, e.g., amino acid substitutions, insertions, or deletions, relative to the amino acid sequence of any one of SEQ ID NO: 1165, SEQ ID NO: 1166, SEQ ID NO: 1167, SEQ ID NO: 473, RVS, and SEQ ID NO: 571.

In some embodiments, the anti-tau antibody comprises at least one, two, three, or all of a heavy chain complementary determining region 1 (HC CDR1), a HC CDR2, and/or a HC CDR3, wherein the HC CDR1, HC CDR2, and HC CDR3 sequences comprise the sequences of SEQ ID NO: 64, 1145, and 1167, respectively. In some embodiments, the anti-tau antibody comprises at least one, two, three, or all of a LC CDR1, a LC CDR2 and/or an LC CDR3, wherein the LC CDR1, LC CDR2, and LC CDR3 sequences comprise the sequences of SEQ ID NO: 1146, 529, and 571, respectively. In some embodiments, the anti-tau antibody comprises a HC CDR1, a HC CDR2, a HC CDR3, a LC CDR1, a LC CDR2 and an LC CDR3, wherein the HC CDR1, HC CDR2, HC CDR3, LC CDR1, LC CDR2, and LC CDR3 sequences comprise the sequences of SEQ ID NO: 64, 1145, 1167, 1146, 529, and 571, respectively. In some embodiments, one or more of the CDRs (or collectively all of the CDRs) have one, two, three, four, five or more changes, e.g., amino acid substitutions, insertions, or deletions, relative to the amino acid sequence of any of SEQ ID NO: 64, 1145, 1167, 1146, 529, and 571.

In some embodiments, the anti-tau antibody comprises at least one, two, three, or all of a heavy chain complementary determining region 1 (HC CDR1), a HC CDR2, and/or a HC CDR3, wherein the HC CDR1, HC CDR2, and HC CDR3 sequences comprise the sequences of SEQ ID NO: 315, 341, and 410, respectively. In some embodiments, the anti-tau antibody comprises at least one, two, three, or all of a LC CDR1, a LC CDR2 and/or an LC CDR3, wherein the LC CDR1, LC CDR2, and LC CDR3 sequences comprise the sequences of SEQ ID NO: 474, 529, and 571, respectively. In some embodiments, the anti-tau antibody comprises a HC CDR1, a HC CDR2, a HC CDR3, a LC CDR1, a LC CDR2 and an LC CDR3, wherein the HC CDR1, HC CDR2, HC CDR3, LC CDR1, LC CDR2, and LC CDR3 sequences comprise the sequences of SEQ ID NO: 315, 341, 410, 474, 529, and 571, respectively. In some embodiments, one or more of the CDRs (or collectively all of the CDRs) have one, two, three, four, five or more changes, e.g., amino acid substitutions, insertions, or deletions, relative to the amino acid sequence of any of SEQ ID NO: 315, 341, 410, 474, 529, and 571.

In some embodiments, the anti-tau antibody comprises at least one, two, three, or all of a heavy chain complementary determining region 1 (HC CDR1), a HC CDR2, and/or a HC CDR3, wherein the HC CDR1, HC CDR2, and HC CDR3 sequences comprise the sequences of SEQ ID NO: 1147, 1148, and 410, respectively. In some embodiments, the anti-tau antibody comprises at least one, two, three, or all of a LC CDR1, a LC CDR2 and/or an LC CDR3, wherein the LC CDR1, LC CDR2, and LC CDR3 sequences comprise the sequences of SEQ ID NO: 474, 529, and 571, respectively. In some embodiments, the anti-tau antibody comprises a HC CDR1, a HC CDR2, a HC CDR3, a LC CDR1, a LC CDR2 and an LC CDR3, wherein the HC CDR1, HC CDR2, HC CDR3, LC CDR1, LC CDR2, and LC CDR3 sequences comprise the sequences of SEQ ID NO: 1147, 1148, 410, 474, 529, and 571, respectively. In some embodiments, one or more of the CDRs (or collectively all of the CDRs) have one, two, three, four, five or more changes, e.g., amino acid substitutions, insertions, or deletions, relative to the amino acid sequence of any of SEQ ID NO: 1147, 1148, 410, 474, 529, and 571.

In some embodiments, the anti-tau antibody comprises at least one, two, three, or all of a heavy chain complementary determining region 1 (HC CDR1), a HC CDR2, and/or a HC CDR3, wherein the HC CDR1, HC CDR2, and HC CDR3 sequences comprise the sequences of SEQ ID NO: 1168, 1169, and 1167, respectively. In some embodiments, the anti-tau antibody comprises at least one, two, three, or all of a LC CDR1, a LC CDR2 and/or an LC CDR3, wherein the LC CDR1, LC CDR2, and LC CDR3 sequences comprise the sequences of SEQ ID NO: 1170, RVS, and SEQ ID NO: 571, respectively. In some embodiments, the anti-tau antibody comprises a HC CDR1, a HC CDR2, a HC CDR3, a LC CDR1, a LC CDR2 and an LC CDR3, wherein the HC CDR1, HC CDR2, HC CDR3, LC CDR1, LC CDR2, and LC CDR3 sequences comprise the sequences of SEQ ID NO: 1168, SEQ ID NO: 1169, SEQ ID NO: 1167, SEQ ID NO: 1170, RVS, and SEQ ID NO: 571, respectively. In some embodiments, one or more of the CDRs (or collectively all of the CDRs) have one, two, three, four, five or more changes, e.g., amino acid substitutions, insertions, or deletions, relative to the amino acid sequence of any of SEQ ID NO: 1168, SEQ ID NO: 1169, SEQ ID NO: 1167, SEQ ID NO: 1170, RVS, and SEQ ID NO: 571.

In some embodiments, the anti-tau antibody comprises at least one, two, three, or all of a heavy chain complementary determining region 1 (HC CDR1), a HC CDR2, and/or a HC CDR3, wherein the HC CDR1, HC CDR2, and HC CDR3 sequences comprise the sequences of SEQ ID NO: 316, 341, and 410, respectively. In some embodiments, the anti-tau antibody comprises at least one, two, three, or all of a LC CDR1, a LC CDR2 and/or an LC CDR3, wherein the LC CDR1, LC CDR2, and LC CDR3 sequences comprise the sequences of SEQ ID NO: 475, 530, and 571, respectively. In some embodiments, the anti-tau antibody comprises a HC CDR1, a HC CDR2, a HC CDR3, a LC CDR1, a LC CDR2 and an LC CDR3, wherein the HC CDR1, HC CDR2, HC CDR3, LC CDR1, LC CDR2, and LC CDR3 sequences comprise the sequences of SEQ ID NO: 316, 341, 410, 475, 530, and 571, respectively. In some embodiments, one or more of the CDRs (or collectively all of the CDRs) have one, two, three, four, five or more changes, e.g., amino acid substitutions, insertions, or deletions, relative to the amino acid sequence of any of SEQ ID NO: 316, 341, 410, 475, 530, and 571.

In some embodiments, the anti-tau antibody comprises at least one, two, three, or all of a heavy chain complementary determining region 1 (HC CDR1), a HC CDR2, and/or a HC CDR3, wherein the HC CDR1, HC CDR2, and HC CDR3 sequences comprise the sequences of SEQ ID NO: 1149, 1150, and 410, respectively. In some embodiments, the anti-tau antibody comprises at least one, two, three, or all of a LC CDR1, a LC CDR2 and/or an LC CDR3, wherein the LC CDR1, LC CDR2, and LC CDR3 sequences comprise the sequences of SEQ ID NO: 475, 530, and 571, respectively. In some embodiments, the anti-tau antibody comprises a HC CDR1, a HC CDR2, a HC CDR3, a LC CDR1, a LC CDR2 and an LC CDR3, wherein the HC CDR1, HC CDR2, HC CDR3, LC CDR1, LC CDR2, and LC CDR3 sequences comprise the sequences of SEQ ID NO: 1149, 1150, 410, 475, 530, and 571, respectively. In some embodiments, one or more of the CDRs (or collectively all of the CDRs) have one, two, three, four, five or more changes, e.g., amino acid substitutions, insertions, or deletions, relative to the amino acid sequence of any of SEQ ID NO: 1149, 1150, 410, 475, 530, and 571.

In some embodiments, the anti-tau antibody comprises at least one, two, three, or all of a heavy chain complementary determining region 1 (HC CDR1), a HC CDR2, and/or a HC CDR3, wherein the HC CDR1, HC CDR2, and HC CDR3 sequences comprise the sequences of SEQ ID NO: 1171, 1166, and 1167, respectively. In some embodiments, the anti-tau antibody comprises at least one, two, three, or all of a LC CDR1, a LC CDR2 and/or an LC CDR3, wherein the LC CDR1, LC CDR2, and LC CDR3 sequences comprise the sequences of SEQ ID NO: 1172, RVS, and SEQ ID NO: 571, respectively. In some embodiments, the anti-tau antibody comprises a HC CDR1, a HC CDR2, a HC CDR3, a LC CDR1, a LC CDR2 and an LC CDR3, wherein the HC CDR1, HC CDR2, HC CDR3, LC CDR1, LC CDR2, and LC CDR3 sequences comprise the sequences of SEQ ID NO: 1171, SEQ ID NO: 1166, SEQ ID NO: 1167, SEQ ID NO: 1172, RVS, and SEQ ID NO: 571, respectively. In some embodiments, one or more of the CDRs (or collectively all of the CDRs) have one, two, three, four, five or more changes, e.g., amino acid substitutions, insertions, or deletions, relative to the amino acid sequence of any of SEQ ID NO: 1171, SEQ ID NO: 1166, SEQ ID NO: 1167, SEQ ID NO: 1172, RVS, and SEQ ID NO: 571.

In some embodiments, the anti-tau antibody comprises at least one, two, three, or all of a heavy chain complementary determining region 1 (HC CDR1), a HC CDR2, and/or a HC CDR3, wherein the HC CDR1, HC CDR2, and HC CDR3 sequences comprise the sequences of SEQ ID NO: 325, 362, and 435, respectively. In some embodiments, the anti-tau antibody comprises at least one, two, three, or all of a LC CDR1, a LC CDR2 and/or an LC CDR3, wherein the LC CDR1, LC CDR2, and LC CDR3 sequences comprise the sequences of SEQ ID NO: 495, 540, and 587, respectively. In some embodiments, the anti-tau antibody comprises a HC CDR1, a HC CDR2, a HC CDR3, a LC CDR1, a LC CDR2 and an LC CDR3, wherein the HC CDR1, HC CDR2, HC CDR3, LC CDR1, LC CDR2, and LC CDR3 sequences comprise the sequences of SEQ ID NO: 325, 362, 435, 495, 540, and 587, respectively. In some embodiments, one or more of the CDRs (or collectively all of the CDRs) have one, two, three, four, five or more changes, e.g., amino acid substitutions, insertions, or deletions, relative to the amino acid sequence of any of SEQ ID NO: 325, 362, 435, 495, 540, and 587.

In some embodiments, the anti-tau antibody comprises at least one, two, three, or all of a heavy chain complementary determining region 1 (HC CDR1), a HC CDR2, and/or a HC CDR3, wherein the HC CDR1, HC CDR2, and HC CDR3 sequences comprise the sequences of SEQ ID NO: 1152, 1153, and 435, respectively. In some embodiments, the anti-tau antibody comprises at least one, two, three, or all of a LC CDR1, a LC CDR2 and/or an LC CDR3, wherein the LC CDR1, LC CDR2, and LC CDR3 sequences comprise the sequences of SEQ ID NO: 495, 540, and 587, respectively. In some embodiments, the anti-tau antibody comprises a HC CDR1, a HC CDR2, a HC CDR3, a LC CDR1, a LC CDR2 and an LC CDR3, wherein the HC CDR1, HC CDR2, HC CDR3, LC CDR1, LC CDR2, and LC CDR3 sequences comprise the sequences of SEQ ID NO: 1152, 1153, 435, 495, 540, and 587, respectively. In some embodiments, one or more of the CDRs (or collectively all of the CDRs) have one, two, three, four, five or more changes, e.g., amino acid substitutions, insertions, or deletions, relative to the amino acid sequence of any of SEQ ID NO: 1152, 1153, 435, 495, 540, and 587.

In some embodiments, the anti-tau antibody comprises at least one, two, three, or all of a heavy chain complementary determining region 1 (HC CDR1), a HC CDR2, and/or a HC CDR3, wherein the HC CDR1, HC CDR2, and HC CDR3 sequences comprise the sequences of SEQ ID NO: 1173, 1174, and 1175, respectively. In some embodiments, the anti-tau antibody comprises at least one, two, three, or all of a LC CDR1, a LC CDR2 and/or an LC CDR3, wherein the LC CDR1, LC CDR2, and LC CDR3 sequences comprise the sequences of SEQ ID NO: 1176, YAS, and SEQ ID NO: 587, respectively. In some embodiments, the anti-tau antibody comprises a HC CDR1, a HC CDR2, a HC CDR3, a LC CDR1, a LC CDR2 and an LC CDR3, wherein the HC CDR1, HC CDR2, HC CDR3, LC CDR1, LC CDR2, and LC CDR3 sequences comprise the sequences of SEQ ID NO: 1173, SEQ ID NO: 1174, SEQ ID NO: 1175, SEQ ID NO: 1176, YAS, and SEQ ID NO: 587, respectively. In some embodiments, one or more of the CDRs (or collectively all of the CDRs) have one, two, three, four, five or more changes, e.g., amino acid substitutions, insertions, or deletions, relative to the amino acid sequence of any of SEQ ID NO: 1173, SEQ ID NO: 1174, SEQ ID NO: 1175, SEQ ID NO: 1176, YAS, and SEQ ID NO: 587.

In some embodiments, the anti-tau antibody comprises a heavy chain variable region comprising the amino acid sequence of SEQ ID NO: 4; or encoded by the nucleotide sequence of SEQ ID NO: 150; or a sequence substantially identical (e.g., having at least about 70%, 75%, 80%, 85%, 90%, 92%, 95%, 97%, 98%, or 99% sequence identity) to any of the aforesaid sequences. In some embodiments, the anti-tau antibody comprises a light chain variable region comprising the amino acid sequence of SEQ ID NO: 78; or encoded by the nucleotide sequence of SEQ ID NO: 224; or a sequence substantially identical (e.g., having at least about 70%, 75%, 80%, 85%, 90%, 92%, 95%, 97%, 98%, or 99% a sequence identity) to any of the aforesaid sequences. In some embodiments, the anti-tau antibody comprises a heavy chain variable region and a light chain variable region comprising the amino acid sequences of SEQ ID NO: 4 and 78, respectively; or encoded by the nucleotide sequences of SEQ ID NO: 150 and 224, respectively; or a sequence substantially identical (e.g., having at least about 70%, 75%, 80%, 85%, 90%, 92%, 95%, 97%, 98%, or 99% sequence identity) to any of the aforesaid sequences. In some embodiments, the nucleotide sequence encoding the heavy chain variable region of the anti-tau antibody comprises the nucleotide sequence of SEQ ID NO: 150, or a sequence substantially identical (e.g., having at least about 80%, 85%, 90%, 92%, 95%, 97%, 98%, or 99% sequence identity); and/or the nucleotide sequence encoding the light chain variable region comprises the nucleotide sequence of SEQ ID NO: 224, or a sequence substantially identical (e.g., having at least about 80%, 85%, 90%, 92%, 95%, 97%, 98%, or 99% sequence identity).

In some embodiments, the anti-tau antibody comprises a heavy chain variable region comprising the amino acid sequence of SEQ ID NO: 9; or encoded by the nucleotide sequence of SEQ ID NO: 155; or a sequence substantially identical (e.g., having at least about 70%, 75%, 80%, 85%, 90%, 92%, 95%, 97%, 98%, or 99% sequence identity) to any of the aforesaid sequences. In some embodiments, the anti-tau antibody comprises a light chain variable region comprising the amino acid sequence of SEQ ID NO: 83; or encoded by the nucleotide sequence of SEQ ID NO: 229; or a sequence substantially identical (e.g., having at least about 70%, 75%, 80%, 85%, 90%, 92%, 95%, 97%, 98%, or 99% sequence identity) to any of the aforesaid sequences. In some embodiments, the anti-tau antibody comprises a heavy chain variable region and a light chain variable region comprising the amino acid sequences of SEQ ID NO: 9 and 83, respectively; or encoded by the nucleotide sequences of SEQ ID NO: 155 and 229, respectively; or a sequence substantially identical (e.g., having at least about 70%, 75%, 80%, 85%, 90%, 92%, 95%, 97%, 98%, or 99% sequence identity) to any of the aforesaid sequences. In some embodiments, the nucleotide sequence encoding the heavy chain variable region of the anti-tau antibody comprises the nucleotide sequence of SEQ ID NO: 155, or a sequence substantially identical (e.g., having at least about 80%, 85%, 90%, 92%, 95%, 97%, 98%, or 99% sequence identity); and/or the nucleotide sequence encoding the light chain variable region comprises the nucleotide sequence of SEQ ID NO: 229, or a sequence substantially identical (e.g., having at least about 80%, 85%, 90%, 92%, 95%, 97%, 98%, or 99% sequence identity).

In some embodiments, the anti-tau antibody comprises a heavy chain variable region comprising the amino acid sequence of SEQ ID NO: 21; or encoded by the nucleotide sequence of SEQ ID NO: 167; or a sequence substantially identical (e.g., having at least about 70%, 75%, 80%, 85%, 90%, 92%, 95%, 97%, 98%, or 99% sequence identity) to any of the aforesaid sequences. In some embodiments, the anti-tau antibody comprises a light chain variable region comprising the amino acid sequence of SEQ ID NO: 93; or encoded by the nucleotide sequence of SEQ ID NO: 241; or a sequence substantially identical (e.g., having at least about 70%, 75%, 80%, 85%, 90%, 92%, 95%, 97%, 98%, or 99% sequence identity) to any of the aforesaid sequences. In some embodiments, the anti-tau antibody comprises a heavy chain variable region and a light chain variable region comprising the amino acid sequences of SEQ ID NO: 21 and 93, respectively; or encoded by the nucleotide sequences of SEQ ID NO: 167 and 241, respectively; or a sequence substantially identical (e.g., having at least about 70%, 75%, 80%, 85%, 90%, 92%, 95%, 97%, 98%, or 99% sequence identity) to any of the aforesaid sequences. In some embodiments, the anti-tau antibody comprises a heavy chain variable region and a light chain variable region comprising the amino acid sequences of SEQ ID NO: 21 and 93, respectively; or encoded by the nucleotide sequences of SEQ ID NO: 7 and 11, respectively; or a sequence substantially identical (e.g., having at least about 70%, 75%, 80%, 85%, 90%, 92%, 95%, 97%, 98%, or 99% sequence identity) to any of the aforesaid sequences. In some embodiments, the nucleotide sequence encoding the heavy chain variable region of the anti-tau antibody comprises the nucleotide sequence of SEQ ID NO: 167, or a sequence substantially identical (e.g., having at least about 80%, 85%, 90%, 92%, 95%, 97%, 98%, or 99% sequence identity); and/or the nucleotide sequence encoding the light chain variable region comprises the nucleotide sequence of SEQ ID NO: 241, or a sequence substantially identical (e.g., having at least about 80%, 85%, 90%, 92%, 95%, 97%, 98%, or 99% sequence identity). In some embodiments, the nucleotide sequence encoding the heavy chain variable region of the anti-tau antibody comprises the nucleotide sequence of SEQ ID NO: 7, or a sequence substantially identical (e.g., having at least about 80%, 85%, 90%, 92%, 95%, 97%, 98%, or 99% sequence identity); and/or the nucleotide sequence encoding the light chain variable region comprises the nucleotide sequence of SEQ ID NO: 11, or a sequence substantially identical (e.g., having at least about 80%, 85%, 90%, 92%, 95%, 97%, 98%, or 99% sequence identity) thereto.

In some embodiments, the antibody that binds to tau comprises a heavy chain variable region comprising the amino acid sequence of any one of SEQ ID NOs: 67-71, or a sequence substantially identical (e.g., having at least about 80%, 85%, 90%, 92%, 95%, 97%, 98%, or 99% sequence identity) to any of the aforesaid sequences. In some embodiments, the antibody comprises a light chain variable region comprising the amino acid sequence of any one of SEQ ID NOs: 72-76, or a sequence substantially identical (e.g., having at least about 80%, 85%, 90%, 92%, 95%, 97%, 98%, or 99% sequence identity) to any of the aforesaid sequences. In some embodiments, the antibody comprises a heavy chain variable region comprising the amino acid sequence of any one of SEQ ID NOs: 67-71, or a sequence substantially identical (e.g., having at least about 80%, 85%, 90%, 92%, 95%, 97%, 98%, or 99% sequence identity) to any of the aforesaid sequences; and a light chain variable region comprising the amino acid sequence of any one of SEQ ID NOs: 72-76, or a sequence substantially identical (e.g., having at least about 80%, 85%, 90%, 92%, 95%, 97%, 98%, or 99% sequence identity) to any of the aforesaid sequences. In some embodiments, the nucleotide sequence encoding a heavy chain variable region of the antibody comprises the nucleotide sequence of any one of SEQ ID NOs: 156-160, or a sequence substantially identical (e.g., having at least about 80%, 85%, 90%, 92%, 95%, 97%, 98%, or 99% sequence identity) to any of the aforesaid sequences. In some embodiments, the nucleotide sequence encoding a light chain variable region of the antibody comprises the nucleotide sequence of any one of SEQ ID NOs: 161-165, or a sequence substantially identical (e.g., having at least about 80%, 85%, 90%, 92%, 95%, 97%, 98%, or 99% sequence identity) to any of the aforesaid sequences. In some embodiments, the nucleotide sequence encoding the heavy chain variable region comprises the nucleotide sequence of any one of SEQ ID NOs: 156-160, or a sequence substantially identical (e.g., having at least about 80%, 85%, 90%, 92%, 95%, 97%, 98%, or 99% sequence identity) to any of the aforesaid sequences; and the nucleotide sequence encoding the light chain variable region comprises the nucleotide sequence of any one of SEQ ID NOs: 161-165, or a sequence substantially identical (e.g., having at least about 80%, 85%, 90%, 92%, 95%, 97%, 98%, or 99% sequence identity) to any of the aforesaid sequences.

In some embodiments, the antibody that binds to tau comprises a heavy chain variable region comprising the amino acid sequence of any one of SEQ ID NOs: 67 or 69-71, or a sequence substantially identical (e.g., having at least about 80%, 85%, 90%, 92%, 95%, 97%, 98%, or 99% sequence identity) to any of the aforesaid sequences. In some embodiments, the antibody comprises a light chain variable region comprising the amino acid sequence of any one of SEQ ID NOs: 72-74, or a sequence substantially identical (e.g., having at least about 80%, 85%, 90%, 92%, 95%, 97%, 98%, or 99% sequence identity) to any of the aforesaid sequences. In some embodiments, the antibody comprises a heavy chain variable region comprising the amino acid sequence of any one of SEQ ID NOs: 67 or 69-71, or a sequence substantially identical (e.g., having at least about 80%, 85%, 90%, 92%, 95%, 97%, 98%, or 99% sequence identity) to any of the aforesaid sequences; and a light chain variable region comprising the amino acid sequence of any one of SEQ ID NOs: 72-74, or a sequence substantially identical (e.g., having at least about 80%, 85%, 90%, 92%, 95%, 97%, 98%, or 99% sequence identity) to any of the aforesaid sequences. In some embodiments, the nucleotide sequence encoding a heavy chain variable region of the antibody comprises the nucleotide sequence of any one of SEQ ID NOs: 156 or 158-160, or a sequence substantially identical (e.g., having at least about 80%, 85%, 90%, 92%, 95%, 97%, 98%, or 99% sequence identity) to any of the aforesaid sequences. In some embodiments, the nucleotide sequence encoding a light chain variable region of the antibody comprises the nucleotide sequence of any one of SEQ ID NOs: 161-163, or a sequence substantially identical (e.g., having at least about 80%, 85%, 90%, 92%, 95%, 97%, 98%, or 99% sequence identity) to any of the aforesaid sequences. In some embodiments, the nucleotide sequence encoding the heavy chain variable region comprises the nucleotide sequence of any one of SEQ ID NOs: 156 or 158-160, or a sequence substantially identical (e.g., having at least about 80%, 85%, 90%, 92%, 95%, 97%, 98%, or 99% sequence identity) to any of the aforesaid sequences; and the nucleotide sequence encoding the light chain variable region comprises the nucleotide sequence of any one of SEQ ID NOs: 161-163, or a sequence substantially identical (e.g., having at least about 80%, 85%, 90%, 92%, 95%, 97%, 98%, or 99% sequence identity) to any of the aforesaid sequences.

In some embodiments, the antibody that binds to tau comprises a heavy chain variable region comprising the amino acid sequence of SEQ ID NO: 69, or a sequence substantially identical (e.g., having at least about 80%, 85%, 90%, 92%, 95%, 97%, 98%, or 99% sequence identity) thereto. In some embodiments, the antibody comprises a light chain variable region comprising the amino acid sequence of any one of SEQ ID NOs: 73, or a sequence substantially identical (e.g., having at least about 80%, 85%, 90%, 92%, 95%, 97%, 98%, or 99% sequence identity) thereto. In some embodiments, the antibody comprises a heavy chain variable region comprising the amino acid sequence of SEQ ID NO: 69, or a sequence substantially identical (e.g., having at least about 80%, 85%, 90%, 92%, 95%, 97%, 98%, or 99% sequence identity) thereto; and a light chain variable region comprising the amino acid sequence of SEQ ID NO: 73, or a sequence substantially identical (e.g., having at least about 80%, 85%, 90%, 92%, 95%, 97%, 98%, or 99% sequence identity) thereto. In some embodiments, the nucleotide sequence encoding a heavy chain variable region of the antibody comprises the nucleotide sequence of SEQ ID NO: 158, or a sequence substantially identical (e.g., having at least about 80%, 85%, 90%, 92%, 95%, 97%, 98%, or 99% sequence identity) thereto. In some embodiments, the nucleotide sequence encoding a light chain variable region of the antibody comprises the nucleotide sequence of SEQ ID NO: 162, or a sequence substantially identical (e.g., having at least about 80%, 85%, 90%, 92%, 95%, 97%, 98%, or 99% sequence identity) thereto. In some embodiments, the nucleotide sequence encoding the heavy chain variable region comprises the nucleotide sequence of SEQ ID NOs: 158, or a sequence substantially identical (e.g., having at least about 80%, 85%, 90%, 92%, 95%, 97%, 98%, or 99% sequence identity) thereto; and the nucleotide sequence encoding the light chain variable region comprises the nucleotide sequence of SEQ ID NO: 162, or a sequence substantially identical (e.g., having at least about 80%, 85%, 90%, 92%, 95%, 97%, 98%, or 99% sequence identity) thereto.

In some embodiments, the anti-tau antibody comprises a heavy chain comprising the amino acid sequence of any one of SEQ ID NOs: 170-174; or encoded by the nucleotide sequence of any one of SEQ ID NOs: 180-184, or a sequence substantially identical (e.g., having at least about 70%, 75%, 80%, 85%, 90%, 92%, 95%, 97%, 98%, or 99% sequence identity) to any of the aforesaid sequences. In some embodiments, the anti-tau antibody comprises a light chain comprising the amino acid sequence of any one of SEQ ID NOs: 175-179; or encoded by the nucleotide sequence of any one of SEQ ID NOs: 185-189, or a sequence substantially identical (e.g., having at least about 70%, 75%, 80%, 85%, 90%, 92%, 95%, 97%, 98%, or 99% sequence identity) to any of the aforesaid sequences. In some embodiments, the antibody comprises a heavy chain comprising the amino acid sequence of any one of SEQ ID NOs: 170-174, or an amino acid sequence substantially identical (e.g., having at least about 70%, 75%, 80%, 85%, 90%, 92%, 95%, 97%, 98%, or 99% sequence identity) to any of the aforesaid sequences; and a light chain comprising the amino acid sequence of any one of SEQ ID NOs: 175-179, or an amino acid sequence substantially identical (e.g., having at least about 70%, 75%, 80%, 85%, 90%, 92%, 95%, 97%, 98%, or 99% sequence identity) to any of the aforesaid sequences. In some embodiments, the nucleotide sequence encoding the heavy chain of the antibody comprises the nucleotide sequence of any one of SEQ ID NOs: 180-184, or a nucleotide sequence substantially identical (e.g., having at least about 70%, 75%, 80%, 85%, 90%, 92%, 95%, 97%, 98%, or 99% sequence identity) to any of the aforesaid sequences; and the nucleotide sequence encoding the light chain comprises the nucleotide sequence of any one of SEQ ID NOs: 185-189, or a nucleotide sequence substantially identical (e.g., having at least about 70%, 75%, 80%, 85%, 90%, 92%, 95%, 97%, 98%, or 99% sequence identity) to any of the aforesaid sequences.

In some embodiments, the anti-tau antibody comprises a heavy chain comprising the amino acid sequence of any one of SEQ ID NOs: 170 or 172-174; or encoded by the nucleotide sequence of any one of SEQ ID NOs: 180-182, or a sequence substantially identical (e.g., having at least about 70%, 75%, 80%, 85%, 90%, 92%, 95%, 97%, 98%, or 99% sequence identity) to any of the aforesaid sequences. In some embodiments, the anti-tau antibody comprises a light chain comprising the amino acid sequence of any one of SEQ ID NOs: 175-177; or encoded by the nucleotide sequence of any one of SEQ ID NOs: 185-187, or a sequence substantially identical (e.g., having at least about 70%, 75%, 80%, 85%, 90%, 92%, 95%, 97%, 98%, or 99% sequence identity) to any of the aforesaid sequences. In some embodiments, the antibody comprises a heavy chain comprising the amino acid sequence of any one of SEQ ID NOs: 170 or 172-174, or an amino acid sequence substantially identical (e.g., having at least about 70%, 75%, 80%, 85%, 90%, 92%, 95%, 97%, 98%, or 99% sequence identity) to any of the aforesaid sequences; and a light chain comprising the amino acid sequence of any one of SEQ ID NOs: 175-177, or an amino acid sequence substantially identical (e.g., having at least about 70%, 75%, 80%, 85%, 90%, 92%, 95%, 97%, 98%, or 99% sequence identity) to any of the aforesaid sequences. In some embodiments, the nucleotide sequence encoding the heavy chain of the antibody comprises the nucleotide sequence of any one of SEQ ID NOs: 180 or 182-184, or a nucleotide sequence substantially identical (e.g., having at least about 70%, 75%, 80%, 85%, 90%, 92%, 95%, 97%, 98%, or 99% sequence identity) to any of the aforesaid sequences; and the nucleotide sequence encoding the light chain comprises the nucleotide sequence of any one of SEQ ID NOs: 185-187, or a nucleotide sequence substantially identical (e.g., having at least about 70%, 75%, 80%, 85%, 90%, 92%, 95%, 97%, 98%, or 99% sequence identity) to any of the aforesaid sequences.

In some embodiments, the anti-tau antibody comprises a heavy chain comprising the amino acid sequence of SEQ ID NO: 172; or encoded by the nucleotide sequence of SEQ ID NO: 182, or a sequence substantially identical (e.g., having at least about 70%, 75%, 80%, 85%, 90%, 92%, 95%, 97%, 98%, or 99% sequence identity) thereto. In some embodiments, the anti-tau antibody comprises a light chain comprising the amino acid sequence of SEQ ID NO: 176; or encoded by the nucleotide sequence of SEQ ID NOs: 186, or a sequence substantially identical (e.g., having at least about 70%, 75%, 80%, 85%, 90%, 92%, 95%, 97%, 98%, or 99% sequence identity) thereto. In some embodiments, the antibody comprises a heavy chain comprising the amino acid sequence of SEQ ID NO: 172, or an amino acid sequence substantially identical (e.g., having at least about 70%, 75%, 80%, 85%, 90%, 92%, 95%, 97%, 98%, or 99% sequence identity) thereto; and a light chain comprising the amino acid sequence of any one of SEQ ID NO: 176, or an amino acid sequence substantially identical (e.g., having at least about 70%, 75%, 80%, 85%, 90%, 92%, 95%, 97%, 98%, or 99% sequence identity) thereto. In some embodiments, the nucleotide sequence encoding the heavy chain of the antibody comprises the nucleotide sequence of SEQ ID NO: 182, or a nucleotide sequence substantially identical (e.g., having at least about 70%, 75%, 80%, 85%, 90%, 92%, 95%, 97%, 98%, or 99% sequence identity) thereto; and the nucleotide sequence encoding the light chain comprises the nucleotide sequence of SEQ ID NO: 186, or a nucleotide sequence substantially identical (e.g., having at least about 70%, 75%, 80%, 85%, 90%, 92%, 95%, 97%, 98%, or 99% sequence identity) thereto.

In some embodiments, the anti-tau antibody comprises a heavy chain comprising the amino acid sequence of SEQ ID NO: 8 or 65; or encoded by the nucleotide sequence of SEQ ID NO: 10 or 191, or a sequence substantially identical (e.g., having at least about 70%, 75%, 80%, 85%, 90%, 92%, 95%, 97%, 98%, or 99% sequence identity) to any of the aforesaid sequences. In some embodiments, the anti-tau antibody comprises a light chain comprising the amino acid sequence of SEQ ID NO: 12 or 66; or encoded by the nucleotide sequence of SEQ ID NO: 13 or 193, or a sequence substantially identical (e.g., having at least about 70%, 75%, 80%, 85%, 90%, 92%, 95%, 97%, 98%, or 99% sequence identity) to any of the aforesaid sequences. In some embodiments, the anti-tau antibody comprises a heavy chain and a light chain comprising the amino acid sequences of SEQ ID NO: 8 and 12, respectively; or a sequence substantially identical (e.g., having at least about 70%, 75%, 80%, 85%, 90%, 92%, 95%, 97%, 98%, or 99% sequence identity) to any of the aforesaid sequences. In some embodiments, the nucleotide sequence encoding the heavy chain comprises the nucleotide sequence of SEQ ID NO: 10 or a sequence substantially identical (e.g., having at least about 70%, 75%, 80%, 85%, 90%, 92%, 95%, 97%, 98%, or 99% sequence identity) thereto; and/or the nucleotide sequence encoding the light chain comprises the nucleotide sequence of SEQ ID NO: 13, or a sequence substantially identical (e.g., having at least about 70%, 75%, 80%, 85%, 90%, 92%, 95%, 97%, 98%, or 99% sequence identity) thereto. In some embodiments, the anti-tau antibody comprises a heavy chain and a light chain comprising the amino acid sequences of SEQ ID NO: 65 and 66, respectively; or a sequence substantially identical (e.g., having at least about 70%, 75%, 80%, 85%, 90%, 92%, 95%, 97%, 98%, or 99% sequence identity) to any of the aforesaid sequences. In some embodiments, the nucleotide sequence encoding the heavy chain comprises the nucleotide sequence of SEQ ID NO: 191 or a sequence substantially identical (e.g., having at least about 70%, 75%, 80%, 85%, 90%, 92%, 95%, 97%, 98%, or 99% sequence identity) thereto; and/or the nucleotide sequence encoding the light chain comprises the nucleotide sequence of SEQ ID NO: 193, or a sequence substantially identical (e.g., having at least about 70%, 75%, 80%, 85%, 90%, 92%, 95%, 97%, 98%, or 99% sequence identity) thereto.

In some embodiments, the anti-tau antibody comprises a heavy chain variable region comprising the amino acid sequence of SEQ ID NO: 22; or encoded by the nucleotide sequence of SEQ ID NO: 168; or a sequence substantially identical (e.g., having at least about 70%, 75%, 80%, 85%, 90%, 92%, 95%, 97%, 98%, or 99% sequence identity) to any of the aforesaid sequences. In some embodiments, the anti-tau antibody comprises a light chain variable region comprising the amino acid sequence of SEQ ID NO: 94; or encoded by the nucleotide sequence of SEQ ID NO: 242; or a sequence substantially identical (e.g., having at least about 70%, 75%, 80%, 85%, 90%, 92%, 95%, 97%, 98%, or 99% sequence identity) to any of the aforesaid sequences. In some embodiments, the anti-tau antibody comprises a heavy chain variable region and a light chain variable region comprising the amino acid sequences of SEQ ID NO: 22 and 94, respectively; or encoded by the nucleotide sequences of SEQ ID NO: 168 and 242, respectively; or a sequence substantially identical (e.g., having at least about 70%, 75%, 80%, 85%, 90%, 92%, 95%, 97%, 98%, or 99% sequence identity) to any of the aforesaid sequences. In some embodiments, the nucleotide sequence encoding the heavy chain variable region of the anti-tau antibody comprises the nucleotide sequence of SEQ ID NO: 168, or a sequence substantially identical (e.g., having at least about 80%, 85%, 90%, 92%, 95%, 97%, 98%, or 99% sequence identity); and/or the nucleotide sequence encoding the light chain variable region comprises the nucleotide sequence of SEQ ID NO: 242, or a sequence substantially identical (e.g., having at least about 80%, 85%, 90%, 92%, 95%, 97%, 98%, or 99% sequence identity).

In some embodiments, the anti-tau antibody comprises a heavy chain variable region comprising the amino acid sequence of SEQ ID NO: 23; or encoded by the nucleotide sequence of SEQ ID NO: 169; or a sequence substantially identical (e.g., having at least about 70%, 75%, 80%, 85%, 90%, 92%, 95%, 97%, 98%, or 99% sequence identity) to any of the aforesaid sequences. In some embodiments, the anti-tau antibody comprises a light chain variable region comprising the amino acid sequence of SEQ ID NO: 95; or encoded by the nucleotide sequence of SEQ ID NO: 243; or a sequence substantially identical (e.g., having at least about 70%, 75%, 80%, 85%, 90%, 92%, 95%, 97%, 98%, or 99% sequence identity) to any of the aforesaid sequences. In some embodiments, the anti-tau antibody comprises a heavy chain variable region and a light chain variable region comprising the amino acid sequences of SEQ ID NO: 23 and 95, respectively; or encoded by the nucleotide sequences of SEQ ID NO: 169 and 243, respectively; or a sequence substantially identical (e.g., having at least about 70%, 75%, 80%, 85%, 90%, 92%, 95%, 97%, 98%, or 99% sequence identity) to any of the aforesaid sequences. In some embodiments, the nucleotide sequence encoding the heavy chain variable region of the anti-tau antibody comprises the nucleotide sequence of SEQ ID NO: 169, or a sequence substantially identical (e.g., having at least about 80%, 85%, 90%, 92%, 95%, 97%, 98%, or 99% sequence identity); and/or the nucleotide sequence encoding the light chain variable region comprises the nucleotide sequence of SEQ ID NO: 243, or a sequence substantially identical (e.g., having at least about 80%, 85%, 90%, 92%, 95%, 97%, 98%, or 99% sequence identity).

In some embodiments, the anti-tau antibody comprises a heavy chain variable region comprising the amino acid sequence of SEQ ID NO: 51; or encoded by the nucleotide sequence of SEQ ID NO: 197; or a sequence substantially identical (e.g., having at least about 70%, 75%, 80%, 85%, 90%, 92%, 95%, 97%, 98%, or 99% sequence identity) to any of the aforesaid sequences. In some embodiments, the anti-tau antibody comprises a light chain variable region comprising the amino acid sequence of SEQ ID NO: 122; or encoded by the nucleotide sequence of SEQ ID NO: 270; or a sequence substantially identical (e.g., having at least about 70%, 75%, 80%, 85%, 90%, 92%, 95%, 97%, 98%, or 99% sequence identity) to any of the aforesaid sequences. In some embodiments, the anti-tau antibody comprises a heavy chain variable region and a light chain variable region comprising the amino acid sequences of SEQ ID NO: 51 and 122, respectively; or encoded by the nucleotide sequences of SEQ ID NO: 197 and 270, respectively; or a sequence substantially identical (e.g., having at least about 70%, 75%, 80%, 85%, 90%, 92%, 95%, 97%, 98%, or 99% sequence identity) to any of the aforesaid sequences. In some embodiments, the nucleotide sequence encoding the heavy chain variable region of the anti-tau antibody comprises the nucleotide sequence of SEQ ID NO: 197, or a sequence substantially identical (e.g., having at least about 80%, 85%, 90%, 92%, 95%, 97%, 98%, or 99% sequence identity); and/or the nucleotide sequence encoding the light chain variable region comprises the nucleotide sequence of SEQ ID NO: 270, or a sequence substantially identical (e.g., having at least about 80%, 85%, 90%, 92%, 95%, 97%, 98%, or 99% sequence identity).

In some embodiments, the anti-tau antibody comprises a VH and/or VL encoded by a codon-optimized nucleic acid sequence. Codon-optimization may be achieved by any method known to one with skill in the art such as, but not limited to, by a method according to Genescript, EMBOSS, Bioinformatics, NUS, NUS2, Geneinfinity, IDT, NUS3, GregThatcher, Insilico, Molbio, N2P, Snapgene, and/or VectorNTI.

In some embodiments, an anti-tau antibody comprises a heavy chain variable region comprising one, two, three, or four framework regions, e.g., one, two, three, or all of a FRH1, FRH2, FRH3, and/or FRH4. In some embodiments, an anti-tau antibody comprises a light chain variable region comprising one, two, three, or four framework regions, e.g., one, two, three, or all of a FRL1, FRL2, FRL3, and/or FRL4. In some embodiments, the heavy chain variable region comprises from N-terminus to C-terminus, FRH1-CDRH1-FRH2-CDRH2-FRH3-CDRH3-FRH4. In some embodiments, the light chain variable region comprises from N terminus to C-terminus, FRL1-CDRL1-FRL2-CDRL2-FRL3-CDRL3-FRL4. In some embodiments, a framework regions is present before a CDR (e.g., a HC or LC CDR1), between two CDRs (e.g., between a CDR1 and CDR2; between a CDR2 and CDR3), and/or after a CDR (e.g., after a CDR3).

In some embodiments, the FRH1 corresponds to positions 1-25 of a heavy chain variable region, numbered according to any one of SEQ ID NOs: 21 or 67-71; the FRH2 corresponds to positions 36-49, numbered according to any one of SEQ ID NOs: 21 or 67-71; the FRH3 corresponds to positions 67-96, numbered according to any one of SEQ ID NOs: 21 or 67-71; and/or the FRH4 corresponds to positions 108-118, numbered according to any one of SEQ ID NOs: 21 or 67-71. In some embodiments, the FRH1 corresponds to positions 1-30 of a heavy chain variable region, numbered according to any one of SEQ ID NOs: 21 or 67-71; the FRH2 corresponds to positions 36-49, numbered according to any one of SEQ ID NOs: 21 or 67-71; the FRH3 corresponds to positions 67-98, numbered according to any one of SEQ ID NOs: 21 or 67-71; and/or the FRH4 corresponds to positions 108-118, numbered according to any one of SEQ ID NOs: 21 or 67-71.

In some embodiments, the FRL1 corresponds to positions 1-23 of a light chain variable region, numbered according to any one of SEQ ID NOs: 72-76 or 93; the FRL2 corresponds to positions 40-54 of a light chain variable region, numbered according to any one of SEQ ID NOs: 72-76 or 93; the FRL3 corresponds to positions 62-93 of a light chain variable region, numbered according to any one of SEQ ID NOs: 72-76 or 93; and/or the FRL4 corresponds to positions 103-112, numbered according to any one of SEQ ID NOs: 72-76 or 93.

In some embodiments, the antibody does not comprise one, two, three, or all of a FRH1 comprising amino acids 1-25 or 1-30 of SEQ ID NO: 21; a FRH2 comprising amino acids 36-49 of SEQ ID NO: 21; an FRH3 comprising amino acids 67-96 or 67-98 of SEQ ID NO: 21; and/or a FRH4 comprising amino acids 108-118 of SEQ ID NO: 21. In some embodiments, the antibody does not comprise one, two, three, or all of a FRL1 comprising amino acids 1-23 of SEQ ID NO: 93; a FRL2 comprising amino acids 40-54 of SEQ ID NO: 93; an FRL3 comprising amino acids 62-93 of SEQ ID NO: 93; and/or a FRL4 comprising amino acids 103-112 of SEQ ID NO: 93.

In some embodiments, the antibody comprises a FRH1 comprising amino acids 1-25 or 1-30 of any one of SEQ ID NOs: 67-71; an amino acid sequence comprising one, two, three but no more than four modifications (e.g., substitutions, e.g., conservative substitutions) relative to amino acids 1-25 or 1-30 of any one of SEQ ID NOs: 67-71; or an amino acid sequence comprising one, two, three, but no more than four different amino acids relative to amino acids 1-25 or 1-30 of any one of SEQ ID NOs: 67-71. In some embodiments, the antibody comprises a FRH2 comprising amino acids 36-49 of any one of SEQ ID NOs: 67-71; an amino acid sequence comprising one, two, three but no more than four modifications (e.g., substitutions, e.g., conservative substitutions) relative to amino acids 36-49 of any one of SEQ ID NOs: 67-71; or an amino acid sequence comprising one, two, three, but no more than four different amino acids relative to amino acids 36-49 of any one of SEQ ID NOs: 67-71. In some embodiments, the antibody comprises a FRH3 comprising amino acids 67-96 or 67-98 of any one of SEQ ID NOs: 67-71; an amino acid sequence comprising one, two, three but no more than four modifications (e.g., substitutions, e.g., conservative substitutions) relative to amino acids 67-96 or 67-98 of any one of SEQ ID NOs: 67-71; or an amino acid sequence comprising one, two, three, but no more than four different amino acids relative to amino acids 67-96 or 67-98 of any one of SEQ ID NOs: 67-71. In some embodiments, the antibody comprises a FRH4 comprising amino acids 108-118 of any one of SEQ ID NOs: 67-71; an amino acid sequence comprising one, two, three but no more than four modifications (e.g., substitutions, e.g., conservative substitutions) relative to amino acids 108-118 of any one of SEQ ID NOs: 67-71; or an amino acid sequence comprising one, two, three, but no more than four different amino acids relative to amino acids 108-118 of any one of SEQ ID NOs: 67-71.

In some embodiments, the antibody comprises a FRL1 comprising amino acids 1-23 of any one of SEQ ID NOs: 72-76; an amino acid sequence comprising one, two, three but no more than four modifications (e.g., substitutions, e.g., conservative substitutions) relative to amino acids 1-23 of any one of SEQ ID NOs: 72-76; or an amino acid sequence comprising one, two, three but no more than four different amino acids relative to amino acids 1-23 of any one of SEQ ID NOs: 72-76. In some embodiments, the antibody comprises a FRL2 comprising amino acids 40-54 of any one of SEQ ID NOs: 72-76; an amino acid sequence comprising one, two, three but no more than four modifications (e.g., substitutions, e.g., conservative substitutions) relative to amino acids 40-54 of any one of SEQ ID NOs: 72-76; or an amino acid sequence comprising one, two, three but no more than four different amino acids relative to amino acids 40-54 of any one of SEQ ID NOs: 72-76. In some embodiments, the antibody comprises a FRL3 comprising amino acids 62-93 of any one of SEQ ID NOs: 72-76; an amino acid sequence comprising one, two, three but no more than four modifications (e.g., substitutions, e.g., conservative substitutions) relative to amino acids 62-93 of any one of SEQ ID NOs: 72-76; or an amino acid sequence comprising one, two, three but no more than four different amino acids relative to amino acids 62-93 of any one of SEQ ID NOs: 72-76. In some embodiments, the antibody comprises a FRL4 comprising amino acids 103-112 of any one of SEQ ID NOs: 72-76; an amino acid sequence comprising one, two, three but no more than four modifications (e.g., substitutions, e.g., conservative substitutions) relative to amino acids 103-112 of any one of SEQ ID NOs: 72-76; or an amino acid sequence comprising one, two, three but no more than four different amino acids relative to amino acids 103-112 of any one of SEQ ID NOs: 72-76.

In some embodiments, an antibody that binds tau does not comprise the amino acid sequence of SEQ ID NO: 21 and/or the amino acid sequence of SEQ ID NO: 93.

Anti-tau antibodies according to the present disclosure may be prepared using any of the antibody sequences (e.g., variable domain amino acid sequences, variable domain amino acid sequence pairs, CDR amino acid sequences, variable domain CDR amino acid sequence sets, variable domain CDR amino acid sequence set pairs, and/or framework region amino acid sequences) presented herein, any may be prepared, for example, as monoclonal antibodies, multispecific antibodies, chimeric antibodies, antibody mimetics, scFvs, or antibody fragments.

In some embodiments, anti-tau antibodies using any of the antibody sequences presented herein may be prepared as IgA, IgD, IgE, IgG, or IgM antibodies. When prepared as mouse IgG antibodies, anti-tau antibodies may be prepared as IgG1, IgG2a, IgG2b, IgG2c, or IgG3 isotypes. When prepared as human IgG antibodies, anti-tau antibodies may be prepared as IgG1, IgG2, IgG3, or IgG4 isotypes. Anti-tau antibodies prepared as human or humanized antibodies may include one or more human constant domains.

This present disclosure provides in some embodiments, a nucleic acid (e.g., an isolated nucleic acid) encoding any of the above described antibodies, and viral genomes, vectors, AAV particles, and cells comprising the same.

### Tau Protein Antigens

In some embodiments, anti-tau antibodies bind to tau protein antigens, e.g., an epitope on a tau protein. Tau protein antigens may include human microtubule-associated protein tau, isoform 2 (SEQ ID NO: 920) or fragments thereof. Tau protein antigens may include ePHF or fragments thereof. Tau protein antigens may include one or more phosphorylated residues. Such phosphorylated residues may correspond to those found with pathological tau. In some embodiments tau protein antigens include any of those listed in Table 8. In the Table, phosphorylated residues associated with each antigen are double-underlined. In some embodiments, tau proteins may include variants (e.g., phosphorylated or unphosphorylated variants) or fragments of the sequences listed. In some embodiments, an antibody that binds to tau described herein binds a phosphorylated residue of a tau protein, e.g., phosphorylated serine at position 422 (e.g., pS422), numbered according to SEQ ID NO: 920. In some embodiments, the antibody preferentially binds, e.g., primarily binds, pathological tau, e.g. PHF-tau, compared to wild-type tau. In some embodiments, the antibody demonstrates superior efficacy in a murine seeding model, e.g., a mouse seeding assay as described in Example

TABLE 8
Tau protein antigen sequences

| | | SEQ |
|---|---|---|
| | | ID |
| Antigen | Sequence | NO |
| human microtubule- | MAEPRQEFEVMEDHAGTYGLGDRKDQGGYTMHQDQEGDTDAGLKESPLQTPTEDG | 920 |
| associated protein | SEEPGSETSDAKSTPTAEDVTAPLVDEGAPGKQAAAQPHTEIPEGTTAEEAGIGD | |
| tau, isoform 2 | TPSLEDEAAGHVTQARMVSKSKDGTGSDDKKAKGADGKTKIATPRGAAPPGQKGQ | |
| | ANATRIPAKTPPAPKTPPSSGEPPKSGDRSGYSSPGSPGTPGSRSRTPSLPTPPT | |
| | REPKKVAVVRTPPKSPSSAKSRLQTAPVPMPDLKNVKSKIGSTENLKHQPGGGKV | |
| | QIINKKLDLSNVQSKCGSKDNIKHVPGGGSVQIVYKPVDLSKVTSKCGSLGNIHH | |
| | KPGGGQVEVKSEKLDFKDRVQSKIGSLDNITHVPGGGNKKIETHKLTFRENAKAK | |
| | TDHGAEIVYKSPVVSGDTSPRHLSNVSSTGSIDMVDSPQLATLADEVSASLAKQG | |
| | L | |
| PT3 epitope peptide | TPGSRSRTPSLPTPPTREPK | 921 |
| (pT212/pT217) | | |
| Peptide 5 | GTPGSRSRTPSLPTPPTRE | 922 |
| (pT212/pS214/pT217) | | |
| Peptide 12 | RENAKAKTDHGAEIVYKSPVVSGDTSPRHLSNVSSTG | 923 |
| (pS396/pS404/pS409) | | |
| Peptide 1 (AT120 | PTREPKKV | 924 |
| epitope) | | |
| 6C5 epitope peptide | ARMVSKS | 925 |
| UCB D & PT76 | SPSSAKSRLQTAPVPMPDLKNVKS | 926 |
| epitope peptide | | |

In some embodiments, anti-tau antibodies of the present disclosure bind to tau protein epitopes on tau protein antigens described herein. Such tau protein epitopes may include or be included within a tau protein antigen amino acid sequence listed in Table 8. In some embodiments, anti-tau antibodies of the present disclosure bind to tau protein epitopes that include a region formed by a complex of at least two tau proteins.

In some embodiments, disclosed herein is an encoded an antibody that competes for binding to tau with the aforesaid antibodies. In some embodiments, disclosed herein is an antibody that binds to the same epitope as, substantially the same epitope as, an epitope that overlaps with, or an epitope that substantially overlaps with, the epitope of the aforesaid anti-tau antibody.

In some embodiment, compete or cross-compete refers to the ability of an antibody to interfere with binding of an anti-tau antibody, e.g., an anti-tau antibody provided herein, to a target, e.g., tau protein. The interference with binding can be direct or indirect (e.g., through an allosteric modulation of the antibody or the target). The extent to which an antibody is able to interfere with the binding of another antibody to the target, and therefore whether it can be said to compete, can be determined using a competition binding assay, for example, a FACS assay, an ELISA or BIACORE assay. In some embodiments, a competition binding assay is a quantitative competition assay. In some embodiments, a first anti-tau antibody is said to compete for binding to the target with a second anti-tau antibody when the binding of the first antibody to the target is reduced by 10% or more, e.g., 20% or more, 30% or more, 40% or more, 50% or more, 55% or more, 60% or more, 65% or more, 70% or more, 75% or more, 80% or more, 85% or more, 90% or more, 95% or more, 98% or more, 99% or more in a competition binding assay (e.g., a competition assay described herein).

In some embodiments, an epitope comprises the moieties of an antigen (e.g., a tau protein antigen) that specifically interact with an antibody. Such moieties, referred to herein as epitopic determinants, typically comprise, or are part of, elements such as amino acid side chains or sugar side chains. An epitopic determinate can be defined by methods known in the art or disclosed herein, e.g., by crystallography or by hydrogen-deuterium exchange. At least one or some of the moieties on the antibody, that specifically interact with an epitopic determinant, are typically located in a CDR(s). Typically, an epitope has a specific three dimensional structural characteristics. Typically, an epitope has specific charge characteristics. Some epitopes are linear epitopes while others are conformational epitopes.

In an embodiment, an epitopic determinant is a moiety on the antigen, e.g., such as amino acid side chain or sugar side chain, or part thereof, which, when the antigen and antibody are co-crystallized, is within a predetermined distance, e.g., within 5 Angstroms, of a moiety on the antibody, referred to herein as a crystallographic epitopic determinant. In some embodiments, the crystallographic epitopic determinants of an epitope are collectively referred to as a crystallographic epitope.

A first antibody binds the same epitope as a second antibody (e.g., a reference antibody, e.g., an antibody disclosed herein) if the first antibody specifically interacts with the same epitopic determinants on the antigen as does the second or reference antibody, e.g., when interaction is measured in the same way for both the antibody and the second or reference antibody. Epitopes that overlap share at least one epitopic determinant. A first antibody binds an overlapping epitope with a second antibody (e.g., a reference antibody, e.g., an antibody disclosed herein) when both antibodies specifically interact with a common epitopic determinant. A first and a second antibody (e.g., a reference antibody, e.g., an antibody disclosed herein) bind substantially overlapping epitopes if at least half of the epitopic determinants of the second or reference antibody are found as epitopic determinants in the epitope of the first antibody. A first and a second antibody (e.g., a reference antibody, e.g., an antibody disclosed herein) bind substantially the same epitope if the first antibody binds at least half of the core epitopic determinants of the epitope of the second or reference antibody, wherein the core epitopic determinants are defined by crystallography.

### Epitope Specificity

Antibodies of the present disclosure may bind to tau protein epitopes, which may include or be included within the residues of SEQ ID NOs: 920-926. Antibodies may compete for binding to tau protein epitopes with other anti-tau antibodies, including, but not limited to, AT100, AT120, PT3, C10.2, P176, IPN002, 6C5, and UCB D. Tau protein epitopes may include C-terminal residues 409-436 of human tau (SEQ ID NO: 920). Such epitopes may include residues 413-430 of human tau (SEQ ID NO: 920). Antibody binding to such residues may exhibit a K_{D} of from about 0.1 nM to about 0.5 nM. In some embodiments, tau protein epitopes may include residues 55-76, 159-194, 219-247, and/or 381-426 of human tau (SEQ ID NO: 920). Such epitopes may include residues 57-72, 175-191, 223-238, and/or 383-400 of human tau (SEQ ID NO: 920). Antibodies binding to such residues may exhibit a K_{D} of from about 0.5 nM to about 5 nM.

In some embodiments, the present disclosure provides antibodies that compete for binding with second antibodies to tau protein epitopes. Such epitopes may include one or more of residues 32-49, 55-76, 57-72, 159-194, 175-191, 185-200, 219-247, 223-238, 381-426, 383-400, 409-436, and 413-430 of human tau (SEQ ID NO: 920).

Tau protein epitopes may include one or more of residues 409-436 and 413-430 of human tau (SEQ ID NO: 920). Second antibodies competing for binding to such epitopes may include variable domain pairs selected from the group consisting of: a VH with the amino acid sequence of SEQ ID NO: 21 and a VL with the amino acid sequence of SEQ ID NO: 93; a VH with the amino acid sequence of SEQ ID NO: 22 and a VL with the amino acid sequence of SEQ ID NO: 94; and a VH with the amino acid sequence of SEQ ID NO: 23 and a VL with the amino acid sequence of SEQ ID NO: 95.

### II. Vectorization

According to the present disclosure, compositions for delivering anti-tau antibodies or functional variants thereof by adeno-associated virus particles (AAVs) are provided. In some embodiments, an AAV particle, e.g., an AAV particle as described herein, or plurality of particles, may be provided, e.g., delivered, via any of several routes of administration, to a cell, tissue, organ, or organism, in vivo, ex vivo, or in vitro.

As used herein, an “AAV particle” is a virus which comprises a capsid and a viral genome with at least one payload region and at least one inverted terminal repeat (ITR) region.

As used herein, “viral genome” or “vector genome” refers to the nucleic acid sequence(s) encapsulated in an AAV particle. Viral genomes comprise at least one payload region encoding polypeptides, e.g., antibodies, antibody-based compositions or fragments thereof.

As used herein, a “payload” or “payload region” is any nucleic acid molecule which encodes one or more polypeptides. At a minimum, a payload region comprises nucleic acid sequences that encode an antibody, an antibody-based composition, or a fragment thereof, but may also optionally comprise one or more functional or regulatory elements to facilitate transcriptional expression and/or polypeptide translation.

In some embodiments, AAV particles, viral genomes and/or payloads, and the methods of their use may be as described in WO2017189963 or WO2020223276, the contents of each of which are herein incorporated by reference in their entirety.

The nucleic acid sequences, viral genomes, and polypeptides disclosed herein may be engineered to contain modular elements and/or sequence motifs assembled to enable expression of an antibody or functional variant thereof, e.g., an antibody described herein. In some embodiments, the nucleic acid sequence encodes an antibody comprising one or more of the CDRs (e.g., heavy chain and/or light chain CDRs) of an antibody, a variable heavy (VH) chain region and/or variable light (VL) chain region, a heavy and/or light chain constant region, or a combination thereof. In some embodiments, the nucleic acid sequence encoding the antibody may also encode a linker, e.g., such that the VH/heavy chain and the VL/light chain of the antibody are connected via a linker. In some embodiments, the viral genome may further comprise a promoter region, an intron, a Kozak sequence, an enhancer, or a polyadenylation sequence. The order of expression, structural position, or concatemer count (e.g., the VH, VL, heavy chain, light chain, and/or linker) may be different within or among different payload regions. The identity, position and number of linkers expressed by payload regions may also vary. In some embodiments, the payload is a region comprising one or more humanized antibody sequences, such as but not limited to, a humanized antibody VL, light chain domain and/or a humanized antibody VH, heavy chain domain, or fragments thereof.

In some embodiments, the present disclosure provides methods for delivering an antibody (e.g., an anti-tau antibody described herein) and/or a nucleic acid sequence encoding an antibody (e.g., an anti-tau antibody described herein) comprised within the viral genome comprised within a recombinant, AAV particle (e.g., an AAV particle described herein) to a cell, tissue, organ, or subject.

### Adeno-Associated Viruses (AAVs) and AAV Particles

In some embodiments, AAV are used as a biological tool due to a relatively simple structure, their ability to infect a wide range of cells (including quiescent and dividing cells) without integration into the host genome and without replicating, and their relatively benign immunogenic profile. In some embodiments, the genome, e.g., viral genome, of the virus may be manipulated to contain a minimum of components for the assembly of a functional recombinant virus, or viral particle, which is loaded with or engineered to target a particular tissue and express or deliver a desired payload, e.g., an antibody (e.g., an anti-tau antibody).

In some embodiments, the AAV, e.g., naturally occurring (e.g., wild-type) AAV or a recombinant AAV, comprises a viral genome which is a linear, single-stranded nucleic acid molecule, e.g., DNA (ssDNA). In some embodiments, the viral genome, e.g., of a naturally occurring (e.g., wild-type) AAV, is approximately 5,000 nucleotides (nt) in length. In some embodiments, inverted terminal repeats (ITRs) traditionally cap the viral genome at both the 5′ and the 3′ end, providing origins of replication for the viral genome. In some embodiments, an AAV viral genome comprises two ITR sequences. In some embodiments, the ITRs have a characteristic T-shaped hairpin structure defined by a self-complementary region (145nt in wild-type AAV) at the 5′ and 3′ ends of the ssDNA which form an energetically stable double stranded region. The double stranded hairpin structures comprise multiple functions including, but not limited to, acting as an origin for DNA replication by functioning as primers for the endogenous DNA polymerase complex of the host viral replication cell.

In some embodiments, the AAV particle, e.g., an AAV particle (e.g., ssAAVs) described herein comprises a viral genome, e.g., viral genome and/or AAV vector, that is self-complementary (scAAV). In some embodiments, the ssAAV comprises nucleic acid molecules, e.g., DNA strands, that anneal together to form double stranded DNA. In some embodiments, a scAAV allows for rapid expression in a transduced cell as it bypasses second strand synthesis.

In some embodiments, the AAV viral genome further comprises nucleotide sequences for two open reading frames, one for the four non-structural Rep proteins (Rep78, Rep68, Rep52, Rep40, encoded by Rep genes) and one for the three capsid, or structural, proteins (VP1, VP2, VP3, encoded by capsid genes or Cap genes). The Rep proteins are used for replication and packaging, while the capsid proteins are assembled to create the protein shell of the AAV particle, or AAV capsid. In some embodiments, alternative splicing and alternate initiation codons and promoters result in the generation of four different Rep proteins from a single open reading frame and the generation of three capsid proteins from a single open reading frame. For example, in some embodiments, for the AAV9/hu.14 serotype (SEQ ID NO: 138 and SEQ ID NO: 137), VP1 refers to amino acids 1-736, VP2 refers to amino acids 138-736, and VP3 refers to amino acids 203-736. In some embodiments, for the amino acid sequence of SEQ ID NO: 3636, VP1 comprises amino acids 1-743, VP2 comprises amino acids 138-742, and VP3 comprises amino acids 203-742. In some embodiments, VP1 is the full-length capsid sequence, while VP2 and VP3 are shorter components of the whole. As a result, changes in the sequence in the VP3 region, are also changes to VP1 and VP2, however, the percent difference as compared to the parent sequence will be greatest for VP3 since it is the shortest sequence of the three. Though described here in relation to the amino acid sequence, the nucleotide sequence encoding these proteins can be similarly described. In some embodiments, the three capsid proteins assemble to create the AAV capsid protein. In some embodiments, the AAV capsid protein typically comprises a molar ratio of 1:1:10 of VP1:VP2:VP3. In some embodiments, the AAV serotype is defined by the AAV capsid. In some instances, the ITRs are also specifically described by the AAV serotype (e.g., AAV2/9).

In some embodiments, a viral genome of a wild-type, e.g., naturally occurring, AAV can be modified to replace the rep/cap sequences with a nucleic acid comprising a transgene encoding a payload, e.g., an antibody or fragment thereof, wherein the viral genome comprises at least one ITR region. In some embodiments, the viral genome of a recombinant AAV comprises two ITR regions, e.g., a 5′ITR or a 3′ITR. In some embodiments, the rep/cap sequences can be provided in trans during production to generate AAV particles. In some embodiments, the viral genome of an AAV is comprised in an AAV vector, which further encodes a capsid protein e.g., a structural protein, wherein the capsid protein comprises a VP1 polypeptide, a VP2 polypeptide, and/or a VP3 polypeptide; and/or a Rep protein, e.g., a non-structural protein, wherein the Rep protein comprises a Rep78 protein, a Rep68, Rep52 protein, and/or a Rep40 protein (e.g., a Rep25 protein and/or a Rep78 protein).

In some embodiments, in addition to the viral genome comprising a nucleic acid encoding a transgene encoding a payload (e.g., an antibody, e.g., an anti-tau antibody), an AAV particle, e.g., an AAV particle described herein, may comprise the viral genome, in whole or in part, of any naturally occurring and/or recombinant AAV serotype nucleotide sequence or variant. In some embodiments, AAV variants may have sequences of significant homology at the nucleic acid (viral genome or capsid) and amino acid levels (capsids), to produce constructs which are generally physical and functional equivalents, replicate by similar mechanisms, and assemble by similar mechanisms. Chiorini et al., J. Vir. 71: 6823-33(1997); Srivastava et al., J. Vir. 45:555-64 (1983); Chiorini et al., J. Vir. 73:1309-1319 (1999); Rutledge et al., J. Vir. 72:309-319 (1998); and Wu et al., J. Vir. 74: 8635-47 (2000), the contents of each of which are incorporated herein by reference in their entirety.

In some embodiments, AAV particles of the present disclosure are recombinant AAV particles which are replication defective and lacking the nucleotide sequences encoding functional Rep and Cap proteins. In some embodiments, these defective AAV particles may lack most or all parental coding sequences and carry only one or two AAV ITR sequences and the nucleic acid of interest for delivery to a cell, a tissue, an organ, or an organism.

In some embodiments, the viral genome or the AAV vector of the AAV particles described herein comprise at least one control element which provides for the replication, transcription, and translation of a coding sequence encoded therein. In some embodiments, a sufficient number of control elements are present such that the coding sequence of the transgene encoded by the viral genome is capable of being replicated, transcribed, and/or translated in a host cell. Non-limiting examples of expression control elements include sequences for transcription initiation and/or termination, promoter and/or enhancer sequences, efficient RNA processing signals such as splicing and polyadenylation signals, sequences that stabilize cytoplasmic mRNA, sequences that enhance translation efficacy (e.g., Kozak consensus sequence), sequences that enhance protein stability, and/or sequences that enhance protein processing and/or secretion.

In some embodiments, the recombinant AAV particles of the present disclosure are capable of providing, e.g., delivering, a transgene to a mammalian cell. In some embodiments, the recombinant AAV particles of the present disclosure are capable of vectorized delivery of an antibody (e.g., an anti-tau antibody) or fragment thereof.

In some embodiments, the AAV particles, vectors, viral genomes, and/or nucleic acids of the present disclosure may be produced recombinantly and may be based on adeno-associated virus (AAV) parent or reference sequences. Methods for producing and/or modifying AAV particles are disclosed in the art such as pseudotyped AAV vectors (PCT Patent Publication Nos. WO200028004; WO200123001; WO2004112727; WO2005005610; and WO2005072364, the content of each of which is incorporated herein by reference in its entirety). In some embodiments, the AAV particles described herein may be modified to enhance the efficiency of delivery, e.g., delivery of a transgene encoding a payload, e.g., an antibody. Without wishing to be bound by theory, it is believed in some embodiments, that a modified, e.g., recombinant, AAV particle can be packaged efficiently and successfully infect target cells at high frequency and with minimal toxicity. In some embodiments, the capsid protein of the AAV particles are engineered according to the methods described in US Publication Number US20130195801, the contents of which are incorporated herein by reference in their entirety.

### AAV Capsids and Variants Thereof

In some embodiments, an AAV particle, e.g., an AAV particle for the vectorized delivery of an antibody or fragment thereof described herein (e.g., an anti-tau antibody), may comprise an AAV capsid variant. In some embodiments, the AAV capsid variant comprises a VOY101 capsid polypeptide or a functional variant thereof, a VOY9P39 capsid polypeptide or a functional variant thereof, a VOY9P33 capsid polypeptide or a functional variant thereof, a AAVPHP.B (PHP.B) capsid polypeptide or a functional variant thereof, a AAVPHP.N (PHP.N) capsid polypeptide or a functional variant thereof, an AAV1 capsid polypeptide or a functional variant thereof, an AAV2 capsid polypeptide or a functional variant thereof, an AAV5 capsid polypeptide or a functional variant thereof, an AAV9 capsid polypeptide or a functional variant thereof, an AAV9 K449R capsid polypeptide or a functional variant thereof, an AAVrh10 capsid polypeptide or a functional variant thereof. In some embodiments, the AAV capsid polypeptide, e.g., AAV capsid variant, comprises an amino acid sequence of any of the AAV capsid polypeptides in Table 2, or an amino acid sequence substantially identical (e.g., having at least 70%, 75%, 80%, 85%, 90%, 92%, 95%, 97%, 98%, or 99% sequence identity) thereto. In some embodiments, the nucleotide sequence encoding the AAV capsid polypeptide or functional variant thereof comprises any one of the nucleotide sequence in Table 2, or a nucleotide sequence substantially identical (e.g., having at least 70%, 75%, 80%, 85%, 90%, 92%, 95%, 97%, 98%, or 99% sequence identity) thereto.

TABLE 2
Exemplary full length capsid sequences

| | SEQ ID | |
|---|---|---|
| Description | NO: | Sequence Information |
| VOY101 | 803 | MAADGYLPDWLEDNLSEGIREWWALKPGAPQPKANQQHQDNARGLVLPGYKYLGPGNGL |
| | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSDGTL |
| | | AVPFKAQAQTGWVQNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKENSKRWNPEIQY |
| | | TSNYYKSNNVEFAVNTEGVYSEPRPIGTRYLTRNL |
| AAV9/hu.14 | 804 | MAADGYLPDWLEDNLSEGIREWWALKPGAPQPKANQQHQDNARGLVLPGYKYLGPGNGL |
| K449R | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVEMIPQYGYLTLNDGSQAVGRSSFYCLEYFPSQMLRTGNNFQF |
| | | SYEFENVPFHSSYAHSQSLDRLMNPLIDQYLYYLSRTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSAQAQ |
| | | AQTGWVQNQGILPGMVWQDRDVYLQGPIWAKIPHTDGNFHPSPLMGGFGMKHPPPQILI |
| | | KNTPVPADPPTAFNKDKLNSFITQYSTGQVSVEIEWELQKENSKRWNPEIQYTSNYYKS |
| | | NNVEFAVNTEGVYSEPRPIGTRYLTRNL |
| AAV9/hu.14 | 138 | MAADGYLPDWLEDNLSEGIREWWALKPGAPQPKANQQHQDNARGLVLPGYKYLGPGNGL |
| WT | | DKGEPVNAADAAALEHDKAYDQQLKAGDNPYLKYNHADAEFQERLKEDTSFGGNLGRAV |
| (amino acid) | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVEMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSAQAQ |
| | | AQTGWVQNQGILPGMVWQDRDVYLQGPIWAKIPHTDGNFHPSPLMGGFGMKHPPPQILI |
| | | KNTPVPADPPTAFNKDKLNSFITQYSTGQVSVEIEWELQKENSKRWNPEIQYTSNYYKS |
| | | NNVEFAVNTEGVYSEPRPIGTRYLTRNL |
| AAV9/hu.14 | 137 | ATGGCTGCCGATGGTTATCTTCCAGATTGGCTCGAGGACAACCTTAGTGAAGGAATTCG |
| WT | | CGAGTGGTGGGCTTTGAAACCTGGAGCCCCTCAACCCAAGGCAAATCAACAACATCAAG |
| (DNA) | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGACCAATACTTGTACTATCTCTCAAAGACTATTAACG |
| | | GTTCTGGACAGAATCAACAAACGCTAAAATTCAGTGTGGCCGGACCCAGCAACATGGCT |
| | | GTCCAGGGAAGAAACTACATACCTGGACCCAGCTACCGACAACAACGTGTCTCAACCAC |
| | | TGTGACTCAAAACAACAACAGCGAATTTGCTTGGCCTGGAGCTTCTTCTTGGGCTCTCA |
| | | ATGGACGTAATAGCTTGATGAATCCTGGACCTGCTATGGCCAGCCACAAAGAAGGAGAG |
| | | GACCGTTTCTTTCCTTTGTCTGGATCTTTAATTTTTGGCAAACAAGGAACTGGAAGAGA |
| | | CAACGTGGATGCGGACAAAGTCATGATAACCAACGAAGAAGAAATTAAAACTACTAACC |
| | | CGGTAGCAACGGAGTCCTATGGACAAGTGGCCACAAACCACCAGAGTGCCCAAGCACAG |
| | | GCGCAGACCGGCTGGGTTCAAAACCAAGGAATACTTCCGGGTATGGTTTGGCAGGACAG |
| | | AGATGTGTACCTGCAAGGACCCATTTGGGCCAAAATTCCTCACACGGACGGCAACTTTC |
| | | ACCCTTCTCCGCTGATGGGAGGGTTTGGAATGAAGCACCCGCCTCCTCAGATCCTCATC |
| | | AAAAACACACCTGTACCTGCGGATCCTCCAACGGCCTTCAACAAGGACAAGCTGAACTC |
| | | TTTCATCACCCAGTATTCTACTGGCCAAGTCAGCGTGGAGATCGAGTGGGAGCTGCAGA |
| | | AGGAAAACAGCAAGCGCTGGAACCCGGAGATCCAGTACACTTCCAACTATTACAAGTCT |
| | | AATAATGTTGAATTTGCTGTTAATACTGAAGGTGTATATAGTGAACCCCGCCCCATTGG |
| | | CACCAGATACCTGACTCGTAATCTGTAA |

In some embodiments, the AAV capsid variant comprises the amino acid sequence of SEQ ID NO: 138 or an amino acid sequence substantially identical (e.g., having at least 70%, 75%, 80%, 85%, 90%, 92%, 95%, 97%, 98%, or 99% sequence identity) thereto. In some embodiments the AAV capsid variant comprises an amino acid sequence comprising at least one, two, or three modifications but no more than 30, 20, or 10 modifications, e.g., substitutions, relative to the amino acid sequence of SEQ ID NO: 138. In some embodiments, the AAV capsid variant comprises an amino acid sequence encoded by the nucleotide sequence of SEQ ID NO: 137 or a nucleotide sequence substantially identical (e.g., having at least 70%, 75%, 80%, 85%, 90%, 92%, 95%, 97%, 98%, or 99% sequence identity) thereto. In some embodiments, the nucleotide sequence encoding the AAV capsid variant comprises the nucleotide sequence of SEQ ID NO: 137 or a nucleotide sequence substantially identical (e.g., having at least 70%, 75%, 80%, 85%, 90%, 92%, 95%, 97%, 98%, or 99% sequence identity) thereto. In some embodiments, the AAV capsid variant comprises substitution at position K449, e.g., a K449R substitution, numbered according to SEQ ID NO: 138.

In some embodiments, the AAV capsid variant comprises a peptide comprising the amino acid sequence of TLAVPFK (SEQ ID NO: 1262). In some embodiments, the peptide is present immediately subsequent to position 588, relative to a reference sequence numbered according to SEQ ID NO: 138. In some embodiments, the capsid polypeptide comprises the amino acid substitutions of A587D and Q588G, numbered according to SEQ ID NO: 138.

In some embodiments, the AAV capsid variant comprises the amino acid substitution of K449R, numbered according to SEQ ID NO: 138; and a peptide comprising the amino acid sequence of TLAVPFK (SEQ ID NO: 1262), wherein the peptide is present immediately subsequent to position 588, relative to a reference sequence numbered according to SEQ ID NO: 138.

In some embodiments, the AAV capsid variant comprises the amino acid substitution of K449R, numbered according to SEQ ID NO: 138; an peptide comprising the amino acid sequence of TLAVPFK (SEQ ID NO: 1262), wherein the insert is present immediately subsequent to position 588, relative to a reference sequence numbered according to SEQ ID NO: 138; and the amino acid substitutions of A587D and Q588G, numbered according to SEQ ID NO: 138.

In some embodiments, the AAV capsid variant comprises a peptide comprising the amino acid sequence of TLAVPFK (SEQ ID NO: 1262), wherein the insert is present immediately subsequent to position 588, relative to a reference sequence numbered according to SEQ ID NO: 138; and the amino acid substitutions of A587D and Q588G, numbered according to SEQ ID NO: 138.

In some embodiments, the AAV capsid variant comprises the amino acid sequence of SEQ ID NO: 804 or an amino acid sequence substantially identical (e.g., having at least 70%, 75%, 80%, 85%, 90%, 92%, 95%, 97%, 98%, or 99% sequence identity) thereto. In some embodiments the AAV capsid variant comprises an amino acid sequence comprising at least one, two, or three modifications but no more than 30, 20, or 10 modifications, e.g., substitutions, relative to the amino acid sequence of SEQ ID NO: 804, optionally wherein position 449 is not R.

In some embodiments, the capsid polypeptide, comprises the amino acid sequence of SEQ ID NO: 803 or an amino acid sequence substantially identical (e.g., having at least 70%, 75%, 80%, 85%, 90%, 92%, 95%, 97%, 98%, or 99% sequence identity) thereto. In some embodiments the AAV capsid variant comprises an amino acid sequence comprising at least one, two, or three modifications but no more than 30, 20, or 10 modifications, e.g., substitutions, relative to the amino acid sequence of SEQ ID NO: 803.

In some embodiments, the AAV capsid variant comprises the amino acid sequence of SEQ ID NO: 138 or an amino acid sequence substantially identical (e.g., having at least 70%, 75%, 80%, 85%, 90%, 92%, 95%, 97%, 98%, or 99% sequence identity) thereto. In some embodiments the AAV capsid variant comprises an amino acid sequence comprising at least one, two, or three modifications but no more than 30, 20, or 10 modifications, e.g., substitutions, relative to the amino acid sequence of SEQ ID NO: 138. In some embodiments, the AAV capsid variant comprises an amino acid sequence encoded by the nucleotide sequence of SEQ ID NO: 137 or a nucleotide sequence substantially identical (e.g., having at least 70%, 75%, 80%, 85%, 90%, 92%, 95%, 97%, 98%, or 99% sequence identity) thereto.

In some embodiments, the AAV capsid variant comprises the amino acid sequence of SEQ ID NO: 804 or an amino acid sequence substantially identical (e.g., having at least 70%, 75%, 80%, 85%, 90%, 92%, 95%, 97%, 98%, or 99% sequence identity) thereto. In some embodiments the AAV capsid variant comprises an amino acid sequence comprising at least one, two, or three modifications but no more than 30, 20, or 10 modifications, e.g., substitutions, relative to the amino acid sequence of SEQ ID NO: 804.

In some embodiments, an AAV particle described herein comprises an AAV capsid variant. In some embodiments, the AAV capsid variant comprises a peptide sequence as described in Table 3. In some embodiments, the AAV capsid variant comprises a peptide sequence as described in WO2021/230987, the contents of which are hereby incorporated by reference in its entirety.

TABLE 3
Exemplary Peptide Sequences

| | SEQ ID | Amino Acid | SEQ ID | |
|---|---|---|---|---|
| Peptide | NO: | Sequence | NO: | Nucleotide Sequence |
| 1 | 3648 | PLNGAVHLY | 3660 | ccgcttaatggtgccgtccatctttat |
| 2 | 3649 | RDSPKGW | 3661 | cgtgattctccgaagggttggca |
| 3 | 3650 | YSTDVRM | 3662 | tattctacggatgtgaggatgca |
| 4 | 3651 | IVMNSLK | 3663 | attgttatgaattcgttgaaggc |
| 5 | 3652 | RESPRGL | 3664 | cgggagagtcctcgtgggctgca |
| 6 | 3653 | SFNDTRA | 3665 | agttttaatgatactagggctca |
| 7 | 3654 | GGTLAVVSL | 3666 | ggtggtacgttggccgtcgtgtcgctt |
| 8 | 3655 | YGLPKGP | 3667 | tatgggttgccgaagggtcct |
| 9 | 3656 | STGTLRL | 3668 | tcgactgggacgcttcggctt |
| 10 | 3657 | YSTDERM | 3669 | tattcgacggatgagaggatg |
| 11 | 3658 | YSTDERK | 3670 | tattcgacggatgagaggaag |
| 12 | 3659 | YVSSVKM | 3671 | tatgtttcgtctgttaagatg |

In some embodiments, the AAV capsid variant comprises at least 3, 4, 5, 6, 7, 8, or 9 consecutive amino acids from the amino acid sequence of any of SEQ ID NO: 3648-3659. In some embodiments, the AAV capsid variant comprises at least 3, 4, 5, 6, 7, 8, or 9 consecutive amino acids from the amino acid sequence of any of SEQ ID NO: 1725-1775, 1785, 1798, or 1819. In some embodiments, the amino acid sequence is present in loop VIII. In some embodiments, the amino acid sequence is present immediately subsequent to position 586, 588, or 589, relative to a reference sequence numbered according to the amino acid sequence of SEQ ID NO: 138.

In some embodiments, the AAV capsid variant comprises an amino acid sequence comprising at least one, two, or three but no more than four modifications, e.g., substitutions, relative to the amino acid sequence of any of SEQ ID NO: 3648-3659. In some embodiments, the AAV capsid variant comprises an amino acid sequence comprising at least one, two, or three but no more than four modifications, e.g., substitutions, relative to the amino acid sequence of any of SEQ ID NO: 1725-1775, 1785, 1798, or 1819. In some embodiments, the amino acid sequence is present in loop VIII. In some embodiments, the amino acid sequence is present immediately subsequent to position 586, 588, or 589, relative to a reference sequence numbered according to the amino acid sequence of SEQ ID NO: 138. In some embodiments, the amino acid sequence replaces positions 587 and 588, numbered according to SEQ ID NO: 138. In some embodiments, the amino acid sequence is present immediately subsequent to position 586 and replaces positions 587 and 588, numbered according to SEQ ID NO: 138.

In some embodiments, the AAV capsid variant comprises the amino acid sequence of PLNGAVHLY (SEQ ID NO: 3648), or an amino acid sequence having at least one, two, or three but no more than four modifications, e.g., substitutions, relative to the amino acid sequence of PLNGAVHLY (SEQ ID NO: 3648), optionally wherein position 7 is H.

In some embodiments, the AAV capsid variant comprises the amino acid sequence of IVMNSLK (SEQ ID NO: 3651), or an amino acid sequence having at least one, two, or three modifications but no more than four modifications, e.g., substitutions, relative to the amino acid sequence of IVMNSLK (SEQ ID NO: 3651).

In some embodiments, the AAV capsid variant comprises the amino acid sequence of any of SEQ ID NO: 1725-3622. In some embodiments, the AAV capsid variant comprises the amino acid sequence of any of SEQ ID NO: 3648-3659. In some embodiments, the amino acid sequence is present in loop VIII of an AAV capsid variant described herein. In some embodiments, the amino acid sequence is present immediately subsequent to position 586, relative to a reference sequence numbered according to the amino acid sequence of SEQ ID NO: 138. In some embodiments, the amino acid sequence is present immediately subsequent to position 588, relative to a reference sequence numbered according to the amino acid sequence of SEQ ID NO: 138. In some embodiments, the amino acid sequence is present immediately subsequent to position 589, relative to a reference sequence numbered according to the amino acid sequence of SEQ ID NO: 138.

In some embodiments, the AAV capsid variant (e.g., an AAV capsid variant described herein), comprises an amino acid sequence encoded by the nucleotide sequence of any one of SEQ ID NOs: 3660-3671, or a nucleotide sequence substantially identical (e.g., having at least 70%, 75%, 80%, 85%, 90%, 92%, 95%, 97%, 98%, or 99% sequence identity) thereto. In some embodiments, the AAV capsid, e.g., an AAV capsid variant described herein, comprises an amino acid sequence encoded by a nucleotide sequence comprising at least one, two, three, four, five, six, or seven modifications but no more than ten modifications of the nucleotide sequences of any of SEQ ID NOs: 3660-3671.

In some embodiments, the nucleotide sequence encoding the AAV capsid variant (e.g., an AAV capsid variant described herein), comprises the nucleotide sequence of any one of SEQ ID NOs: 3660-3671, or a nucleotide sequence substantially identical (e.g., having at least 70%, 75%, 80%, 85%, 90%, 92%, 95%, 97%, 98%, or 99% sequence identity) thereto. In some embodiments, nucleic acid sequence encoding the AAV capsid variant, e.g., an AAV capsid variant described herein, comprises a nucleotide sequence comprising at least one, two, three, four, five, six, or seven modifications but no more than ten modifications of the nucleotide sequences of any of SEQ ID NOs: 3660-3671.

In some embodiments, the nucleotide sequence encoding the AAV capsid variant (e.g., an AAV capsid variant described herein), comprises the nucleotide sequence of SEQ ID NO: 3660, or a nucleotide sequence substantially identical (e.g., having at least 70%, 75%, 80%, 85%, 90%, 92%, 95%, 97%, 98%, or 99% sequence identity) thereto. In some embodiments, the nucleic acid sequence encoding the AAV capsid variant comprises a nucleotide sequence comprising at least one, two, three, four, five, six, or seven modifications but no more than ten modifications of the nucleotide sequences of SEQ ID NO: 3660.

In some embodiments, the nucleotide sequence encoding the AAV capsid variant (e.g., an AAV capsid variant described herein), comprises the nucleotide sequence of SEQ ID NO: 3663, or a nucleotide sequence substantially identical (e.g., having at least 70%, 75%, 80%, 85%, 90%, 92%, 95%, 97%, 98%, or 99% sequence identity) thereto. In some embodiments, the nucleic acid sequence encoding the AAV capsid variant comprises a nucleotide sequence comprising at least one, two, three, four, five, six, or seven modifications but no more than ten modifications of the nucleotide sequences of SEQ ID NO: 3663.

In some embodiments, the AAV capsid variant comprises an amino acid residue other than “A” at position 587 and/or an amino acid residue other than “Q” at position 588, numbered according to SEQ ID NO: 138. In some embodiments, the AAV capsid variant comprises the amino acid P at position 587 and the amino acid L at position 588, numbered according to SEQ ID NO: 138. In some embodiments, the AAV capsid variant comprises the amino acid G at position 587 and the amino acid G at position 588, numbered according to SEQ ID NO: 138. In some embodiments, the AAV capsid variant comprises the substitutions A587P and Q588L, numbered according to SEQ ID NO: 138. In some embodiments, the AAV capsid variant comprises the substitutions A587G and Q588G, numbered according to SEQ ID NO: 138.

In some embodiments, the AAV capsid variant comprises the amino acid sequence of PLNGAVHLY (SEQ ID NO: 3648), wherein the amino acid sequence of PLNGAVHLY (SEQ ID NO: 3648) is present immediately subsequent to position 586, numbered according to the amino acid sequence of SEQ ID NO: 138. In some embodiments, the AAV capsid variant comprises the amino acid sequence of PLNGAVHLY (SEQ ID NO: 3648) wherein the amino acid sequence of PLNGAVHLY (SEQ ID NO: 3648) replaces positions 587 and 588, numbered according to the amino acid sequence of SEQ ID NO: 138. In some embodiments, the AAV capsid variant comprises the amino acid sequence of PLNGAVHLY (SEQ ID NO: 3648) wherein the amino acid sequence of PLNGAVHLY (SEQ ID NO: 3648) is present immediately subsequent to position 586 and replaces positions 587 and 588, numbered according to SEQ ID NO: 138. In some embodiments, the AAV capsid variant comprises the amino acid sequence of PLNGAVHLY (SEQ ID NO: 3648), wherein the amino acid sequence of PLNGAVHLY (SEQ ID NO: 3648) is present immediately subsequent to position 586, numbered according to the amino acid sequence of SEQ ID NO: 138, and further comprises a deletion of the amino acids AQ at positions 587-588, numbered according to SEQ ID NO: 138.

In some embodiments, the AAV capsid variant comprises the amino acid P at position 587, the amino acid L at position 588, and further comprises the amino acid sequence NGAVHLY (SEQ ID NO: 800), which is present immediately subsequent to position 588, numbered according to SEQ ID NO: 138.

In some embodiments, the AAV capsid variant comprises the amino acid sequence of GGTLAVVSL (SEQ ID NO: 3654), wherein the amino acid sequence of GGTLAVVSL (SEQ ID NO: 3654) is present immediately subsequent to position 586, numbered according to the amino acid sequence of SEQ ID NO: 138. In some embodiments, the AAV capsid variant comprises the amino acid sequence of GGTLAVVSL (SEQ ID NO: 3654), wherein the amino acid sequence of GGTLAVVSL (SEQ ID NO: 3654) replaces positions 587 and 588, numbered according to the amino acid sequence of SEQ ID NO: 138. In some embodiments, the AAV capsid variant comprises the amino acid sequence of GGTLAVVSL (SEQ ID NO: 3654), wherein the amino acid sequence of GGTLAVVSL (SEQ ID NO: 3654) is present immediately subsequent to position 586 and replaces positions 587 and 588, numbered according to SEQ ID NO: 138. In some embodiments, the AAV capsid variant comprises the amino acid sequence of GGTLAVVSL (SEQ ID NO: 3654), wherein the amino acid sequence of GGTLAVVSL (SEQ ID NO: 3654) is present immediately subsequent to position 586, numbered according to the amino acid sequence of SEQ ID NO: 138, and further comprises a deletion of the amino acids AQ at positions 587-588, numbered according to SEQ ID NO: 138.

In some embodiments, the AAV capsid variant comprises the amino acid G at position 587, the amino acid G at position 588, and further comprises the amino acid sequence TLAVVSL (SEQ ID NO: 801), which is present immediately subsequent to position 588, numbered according to SEQ ID NO: 138.

In some embodiments, the AAV capsid variant comprises the amino acid sequence of IVMNSLK (SEQ ID NO: 3651), wherein the amino acid sequence of IVMNSLK (SEQ ID NO: 3651) is present immediately subsequent to position 588, relative to a reference sequence numbered according to the amino acid sequence of SEQ ID NO: 138.

In some embodiments, the AAV capsid variant comprises the amino acid sequence of any of SEQ ID NOs: 3649, 3650, 3652, 3653, or 3655-3659, wherein the amino acid sequence of any of the aforesaid sequences is present immediately subsequent to position 589, relative to a reference sequence numbered according to the amino acid sequence of SEQ ID NO: 138.

In some embodiments, the AAV capsid variant further comprises a substitution at position K449, e.g., a K449R substitution, numbered according to SEQ ID NO: 138. In some embodiments, the AAV capsid variant further comprises a modification, e.g., an insertion, substitution, and/or deletion in loop I, II, IV, and/or VI.

In some embodiments, the AAV capsid variant further comprises an amino acid sequence having at least one, two or three modifications but not more than 30, 20 or 10 modifications of the amino acid sequence of SEQ ID NO: 138. In some embodiments, the AAV capsid variant further comprises the amino acid sequence of SEQ ID NO: 138, or an amino acid sequence with at least 80% (e.g., at least about 85, 90, 95, 96, 97, 98, or 99%) sequence identity thereto. In some embodiments, the AAV capsid variant further comprises an amino acid sequence encoded by the nucleotide sequence of SEQ ID NO: 137, or a sequence with at least 80% (e.g., at least about 85, 90, 95, 96, 97, 98, or 99%) sequence identity thereto.

In some embodiments, an AAV capsid variant of the present disclosure comprises an amino acid sequence as described herein, e.g. an amino acid sequence of an AAV capsid variant chosen from TTD-001, TTD-002, TTD-003, TTD-004, TTD-005, TTD-006, TTD-007, TTD-008, TTD-009, TTD-010, TTD-011, or TTD-012, e.g., as described in Tables 6 and 7. In some embodiments, the AAV capsid variant comprises a sequence as described in WO2021/230987, the contents of which are hereby incorporated by reference in its entirety.

In some embodiments, an AAV capsid variant comprises a VP 1, VP2, and/or VP3 protein comprising an amino acid sequence described herein, e.g., an amino acid sequence of an AAV capsid variant chosen from TTD-001, TTD-002, TTD-003, TTD-004, TTD-005, TTD-006, TTD-007, TTD-008, TTD-009, TTD-10, TTD-011, or TTD-012, e.g., as described in Tables 6 and 7.

In some embodiments, an AAV capsid variant described herein comprises an amino acid sequence encoded by a nucleotide sequence as described herein, e.g., a nucleotide sequence of an AAV capsid variant chosen from TTD-01, TTD-002, TTD-003, TTD-004, TTD-005, TTD-006, TTD-007, TTD-008, TD-009, TTD-010, TTD-011, or TTD-012, e.g., as described in Tables 6 and 9.

In some embodiments, a polynucleotide encoding an AAV capsid variant of the present disclosure comprises a nucleotide sequence described herein, e.g., a nucleotide sequence of an AAV capsid variant chosen from TTD-01, TTD-002, TTD-003, TTD-004, TTD-005, TTD-006, TTD-007, TTD-008, TTD-009, TTD-10, TTD-011, or TTD-012, e.g., as described in Tables 6 and 9.

In some embodiments, insertion of a nucleic acid sequence, targeting nucleic acid sequence, or a peptide into a parent AAV sequence generates the non-limiting exemplary full length capsid sequences, e.g., an AAV capsid variant, as described in Tables 6, 7, and 9.

TABLE 6
Exemplary full length capsid sequences

| | VP1 DNA | VP1 Amino Acid | Peptide | DNA sequence encoding |
|---|---|---|---|---|
| Name | SEQ ID NO: | SEQ ID NO: | SEQ ID NO: | Peptide SEQ ID NO: |
| TTD-001 | 3623 | 3636 | 3648 | 3660 |
| TTD-002 | 3624 or 3625 | 3637 | 3649 | 3661 |
| TTD-003 | 3626 | 3638 | 3650 | 3662 |
| TTD-004 | 3627 | 3639 | 3651 | 3663 |
| TTD-005 | 3628 | 3640 | 3652 | 3664 |
| TTD-006 | 3629 | 3641 | 3653 | 3665 |
| TTD-007 | 3630 | 3642 | 3654 | 3666 |
| TTD-008 | 3631 | 3643 | 3655 | 3667 |
| TTD-009 | 3632 | 3644 | 3656 | 3668 |
| TTD-010 | 3633 | 3645 | 3657 | 3669 |
| TTD-011 | 3634 | 3646 | 3658 | 3670 |
| TTD-012 | 3635 | 3647 | 3659 | 3671 |

TABLE 7
Exemplary full length capsid amino acid sequences

| Name and | SEQ ID | |
|---|---|---|
| Annotation | NO: | Amino Acid Sequence |
| TTD-001 | 3636 | MAADGYLPDWLEDNLSEGIREWWALKPGAPQPKANQQHQDNARGLVLPGYKYLG |
| 9 mer peptide | | PGNGLDKGEPVNAADAAALEHDKAYDQQLKAGDNPYLKYNHADAEFQERLKEDT |
| underlined, starts at | | SFGGNLGRAVFQAKKRLLEPLGLVEEAAKTAPGKKRPVEQSPQEPDSSAGIGKS |
| position 587 | | GAQPAKKRLNFGQTGDTESVPDPQPIGEPPAAPSGVGSLTMASGGGAPVADNNE |
| (immediately | | GADGVGSSSGNWHCDSQWLGDRVITTSTRTWALPTYNNHLYKQISNSTSGGSSN |
| subsequent to | | DNAYFGYSTPWGYFDFNRFHCHFSPRDWQRLINNNWGFRPKRLNFKLFNIQVKE |
| position 586); | | VTDNNGVKTIANNLTSTVQVFTDSDYQLPYVLGSAHEGCLPPFPADVFMIPQYG |
| 743 aa | | YLTLNDGSQAVGRSSFYCLEYFPSQMLRTGNNFQFSYEFENVPFHSSYAHSQSL |
| | | DRLMNPLIDQYLYYLSKTINGSGQNQQTLKFSVAGPSNMAVQGRNYIPGPSYRQ |
| | | QRVSTTVTQNNNSEFAWPGASSWALNGRNSLMNPGPAMASHKEGEDRFFPLSGS |
| | | LIFGKQGTGRDNVDADKVMITNEEEIKTTNPVATESYGQVATNHQSPLNGAVHL |
| | | YAQAQTGWVQNQGILPGMVWQDRDVYLQGPIWAKIPHTDGNFHPSPLMGGFGMK |
| | | HPPPQILIKNTPVPADPPTAFNKDKLNSFITQYSTGQVSVEIEWELQKENSKRW |
| | | NPEIQYTSNYYKSNNVEFAVNTEGVYSEPRPIGTRYLTRNL |
| TTD-002 | 3637 | MAADGYLPDWLEDNLSEGIREWWALKPGAPQPKANQQHQDNARGLVLPGYKYLG |
| 7 mer peptide | | PGNGLDKGEPVNAADAAALEHDKAYDQQLKAGDNPYLKYNHADAEFQERLKEDT |
| underlined, starts at | | SFGGNLGRAVFQAKKRLLEPLGLVEEAAKTAPGKKRPVEQSPQEPDSSAGIGKS |
| position 590 | | GAQPAKKRLNFGQTGDTESVPDPQPIGEPPAAPSGVGSLTMASGGGAPVADNNE |
| (immediately | | GADGVGSSSGNWHCDSQWLGDRVITTSTRTWALPTYNNHLYKQISNSTSGGSSN |
| subsequent to | | DNAYFGYSTPWGYFDFNRFHCHFSPRDWQRLINNNWGFRPKRLNFKLFNIQVKE |
| position 589); | | VTDNNGVKTIANNLTSTVQVFTDSDYQLPYVLGSAHEGCLPPFPADVFMIPQYG |
| 743 aa | | YLTLNDGSQAVGRSSFYCLEYFPSQMLRTGNNFQFSYEFENVPFHSSYAHSQSL |
| | | DRLMNPLIDQYLYYLSKTINGSGQNQQTLKFSVAGPSNMAVQGRNYIPGPSYRQ |
| | | QRVSTTVTQNNNSEFAWPGASSWALNGRNSLMNPGPAMASHKEGEDRFFPLSGS |
| | | LIFGKQGTGRDNVDADKVMITNEEEIKTTNPVATESYGQVATNHQSAQARDSPK |
| | | GWQAQTGWVQNQGILPGMVWQDRDVYLQGPIWAKIPHTDGNFHPSPLMGGFGMK |
| | | HPPPQILIKNTPVPADPPTAFNKDKLNSFITQYSTGQVSVEIEWELQKENSKRW |
| | | NPEIQYTSNYYKSNNVEFAVNTEGVYSEPRPIGTRYLTRNL |
| TTD-003 | 3638 | MAADGYLPDWLEDNLSEGIREWWALKPGAPQPKANQQHQDNARGLVLPGYKYLG |
| 7 mer peptide | | PGNGLDKGEPVNAADAAALEHDKAYDQQLKAGDNPYLKYNHADAEFQERLKEDT |
| underlined, starts at | | SFGGNLGRAVFQAKKRLLEPLGLVEEAAKTAPGKKRPVEQSPQEPDSSAGIGKS |
| position 590 | | GAQPAKKRLNFGQTGDTESVPDPQPIGEPPAAPSGVGSLTMASGGGAPVADNNE |
| (immediately | | GADGVGSSSGNWHCDSQWLGDRVITTSTRTWALPTYNNHLYKQISNSTSGGSSN |
| subsequent to | | DNAYFGYSTPWGYFDFNRFHCHFSPRDWQRLINNNWGFRPKRLNFKLFNIQVKE |
| position 589); | | VTDNNGVKTIANNLTSTVQVFTDSDYQLPYVLGSAHEGCLPPFPADVEMIPQYG |
| 743 aa | | YLTLNDGSQAVGRSSFYCLEYFPSQMLRTGNNFQFSYEFENVPFHSSYAHSQSL |
| | | DRLMNPLIDQYLYYLSKTINGSGQNQQTLKFSVAGPSNMAVQGRNYIPGPSYRQ |
| | | QRVSTTVTQNNNSEFAWPGASSWALNGRNSLMNPGPAMASHKEGEDRFFPLSGS |
| | | LIFGKQGTGRDNVDADKVMITNEEEIKTTNPVATESYGQVATNHQSAQAYSTDV |
| | | RMQAQTGWVQNQGILPGMVWQDRDVYLQGPIWAKIPHTDGNFHPSPLMGGFGMK |
| | | HPPPQILIKNTPVPADPPTAFNKDKLNSFITQYSTGQVSVEIEWELQKENSKRW |
| | | NPEIQYTSNYYKSNNVEFAVNTEGVYSEPRPIGTRYLTRNL |
| TTD-004 | 3639 | MAADGYLPDWLEDNLSEGIREWWALKPGAPQPKANQQHQDNARGLVLPGYKYLG |
| 7 mer peptide | | PGNGLDKGEPVNAADAAALEHDKAYDQQLKAGDNPYLKYNHADAEFQERLKEDT |
| underlined, starts at | | SFGGNLGRAVFQAKKRLLEPLGLVEEAAKTAPGKKRPVEQSPQEPDSSAGIGKS |
| position 589 | | GAQPAKKRLNFGQTGDTESVPDPQPIGEPPAAPSGVGSLTMASGGGAPVADNNE |
| (immediately | | GADGVGSSSGNWHCDSQWLGDRVITTSTRTWALPTYNNHLYKQISNSTSGGSSN |
| subsequent to | | DNAYFGYSTPWGYFDFNRFHCHFSPRDWQRLINNNWGFRPKRLNFKLFNIQVKE |
| position 588); | | VTDNNGVKTIANNLTSTVQVFTDSDYQLPYVLGSAHEGCLPPFPADVEMIPQYG |
| 743 aa | | YLTLNDGSQAVGRSSFYCLEYFPSQMLRTGNNFQFSYEFENVPFHSSYAHSQSL |
| | | DRLMNPLIDQYLYYLSKTINGSGQNQQTLKFSVAGPSNMAVQGRNYIPGPSYRQ |
| | | QRVSTTVTQNNNSEFAWPGASSWALNGRNSLMNPGPAMASHKEGEDRFFPLSGS |
| | | LIFGKQGTGRDNVDADKVMITNEEEIKTTNPVATESYGQVATNHQSAQIVMNSL |
| | | KAQAQTGWVQNQGILPGMVWQDRDVYLQGPIWAKIPHTDGNFHPSPLMGGFGMK |
| | | HPPPQILIKNTPVPADPPTAFNKDKLNSFITQYSTGQVSVEIEWELQKENSKRW |
| | | NPEIQYTSNYYKSNNVEFAVNTEGVYSEPRPIGTRYLTRNL |
| TTD-005 | 3640 | MAADGYLPDWLEDNLSEGIREWWALKPGAPQPKANQQHQDNARGLVLPGYKYLG |
| 7 mer peptide | | PGNGLDKGEPVNAADAAALEHDKAYDQQLKAGDNPYLKYNHADAEFQERLKEDT |
| underlined, starts at | | SFGGNLGRAVFQAKKRLLEPLGLVEEAAKTAPGKKRPVEQSPQEPDSSAGIGKS |
| position 590 | | GAQPAKKRLNFGQTGDTESVPDPQPIGEPPAAPSGVGSLTMASGGGAPVADNNE |
| (immediately | | GADGVGSSSGNWHCDSQWLGDRVITTSTRTWALPTYNNHLYKQISNSTSGGSSN |
| subsequent to | | DNAYFGYSTPWGYFDFNRFHCHFSPRDWQRLINNNWGFRPKRLNFKLFNIQVKE |
| position 589); | | VTDNNGVKTIANNLTSTVQVFTDSDYQLPYVLGSAHEGCLPPFPADVFMIPQYG |
| 743 aa | | YLTLNDGSQAVGRSSFYCLEYFPSQMLRTGNNFQFSYEFENVPFHSSYAHSQSL |
| | | DRLMNPLIDQYLYYLSKTINGSGQNQQTLKFSVAGPSNMAVQGRNYIPGPSYRQ |
| | | QRVSTTVTQNNNSEFAWPGASSWALNGRNSLMNPGPAMASHKEGEDRFFPLSGS |
| | | LIFGKQGTGRDNVDADKVMITNEEEIKTTNPVATESYGQVATNHQSAQARESPR |
| | | GLQAQTGWVQNQGILPGMVWQDRDVYLQGPIWAKIPHTDGNFHPSPLMGGFGMK |
| | | HPPPQILIKNTPVPADPPTAFNKDKLNSFITQYSTGQVSVEIEWELQKENSKRW |
| | | NPEIQYTSNYYKSNNVEFAVNTEGVYSEPRPIGTRYLTRNL |
| TTD-006 | 3641 | MAADGYLPDWLEDNLSEGIREWWALKPGAPQPKANQQHQDNARGLVLPGYKYLG |
| 7 mer peptide | | PGNGLDKGEPVNAADAAALEHDKAYDQQLKAGDNPYLKYNHADAEFQERLKEDT |
| underlined, starts at | | SFGGNLGRAVFQAKKRLLEPLGLVEEAAKTAPGKKRPVEQSPQEPDSSAGIGKS |
| position 590 | | GAQPAKKRLNFGQTGDTESVPDPQPIGEPPAAPSGVGSLTMASGGGAPVADNNE |
| (immediately | | GADGVGSSSGNWHCDSQWLGDRVITTSTRTWALPTYNNHLYKQISNSTSGGSSN |
| subsequent to 589); | | DNAYFGYSTPWGYFDFNRFHCHFSPRDWQRLINNNWGFRPKRLNFKLFNIQVKE |
| 743 aa | | VTDNNGVKTIANNLTSTVQVFTDSDYQLPYVLGSAHEGCLPPFPADVEMIPQYG |
| | | YLTLNDGSQAVGRSSFYCLEYFPSQMLRTGNNFQFSYEFENVPFHSSYAHSQSL |
| | | DRLMNPLIDQYLYYLSKTINGSGQNQQTLKFSVAGPSNMAVQGRNYIPGPSYRQ |
| | | QRVSTTVTQNNNSEFAWPGASSWALNGRNSLMNPGPAMASHKEGEDRFFPLSGS |
| | | LIFGKQGTGRDNVDADKVMITNEEEIKTTNPVATESYGQVATNHQSAQASFNDT |
| | | RAQAQTGWVQNQGILPGMVWQDRDVYLQGPIWAKIPHTDGNFHPSPLMGGFGMK |
| | | HPPPQILIKNTPVPADPPTAFNKDKLNSFITQYSTGQVSVEIEWELQKENSKRW |
| | | NPEIQYTSNYYKSNNVEFAVNTEGVYSEPRPIGTRYLTRNL |
| TTD-007 | 3642 | MAADGYLPDWLEDNLSEGIREWWALKPGAPQPKANQQHQDNARGLVLPGYKYLG |
| 9 mer peptide | | PGNGLDKGEPVNAADAAALEHDKAYDQQLKAGDNPYLKYNHADAEFQERLKEDT |
| underlined, starts at | | SFGGNLGRAVFQAKKRLLEPLGLVEEAAKTAPGKKRPVEQSPQEPDSSAGIGKS |
| position 587 | | GAQPAKKRLNFGQTGDTESVPDPQPIGEPPAAPSGVGSLTMASGGGAPVADNNE |
| (immediately | | GADGVGSSSGNWHCDSQWLGDRVITTSTRTWALPTYNNHLYKQISNSTSGGSSN |
| subsequent to | | DNAYFGYSTPWGYFDFNRFHCHFSPRDWQRLINNNWGFRPKRLNFKLFNIQVKE |
| position 586); | | VTDNNGVKTIANNLTSTVQVFTDSDYQLPYVLGSAHEGCLPPFPADVEMIPQYG |
| 743 aa | | YLTLNDGSQAVGRSSFYCLEYFPSQMLRTGNNFQFSYEFENVPFHSSYAHSQSL |
| | | DRLMNPLIDQYLYYLSKTINGSGQNQQTLKFSVAGPSNMAVQGRNYIPGPSYRQ |
| | | QRVSTTVTQNNNSEFAWPGASSWALNGRNSLMNPGPAMASHKEGEDRFFPLSGS |
| | | LIFGKQGTGRDNVDADKVMITNEEEIKTTNPVATESYGQVATNHQSGGTLAVVS |
| | | LAQAQTGWVQNQGILPGMVWQDRDVYLQGPIWAKIPHTDGNFHPSPLMGGFGMK |
| | | HPPPQILIKNTPVPADPPTAFNKDKLNSFITQYSTGQVSVEIEWELQKENSKRW |
| | | NPEIQYTSNYYKSNNVEFAVNTEGVYSEPRPIGTRYLTRNL |
| TTD-008 | 3643 | MAADGYLPDWLEDNLSEGIREWWALKPGAPQPKANQQHQDNARGLVLPGYKYLG |
| 7 mer peptide | | PGNGLDKGEPVNAADAAALEHDKAYDQQLKAGDNPYLKYNHADAEFQERLKEDT |
| underlined, starts at | | SFGGNLGRAVFQAKKRLLEPLGLVEEAAKTAPGKKRPVEQSPQEPDSSAGIGKS |
| position 590 | | GAQPAKKRLNFGQTGDTESVPDPQPIGEPPAAPSGVGSLTMASGGGAPVADNNE |
| (immediately | | GADGVGSSSGNWHCDSQWLGDRVITTSTRTWALPTYNNHLYKQISNSTSGGSSN |
| subsequent to 589); | | DNAYFGYSTPWGYFDFNRFHCHFSPRDWQRLINNNWGFRPKRLNFKLFNIQVKE |
| 743 aa | | VTDNNGVKTIANNLTSTVQVFTDSDYQLPYVLGSAHEGCLPPFPADVFMIPQYG |
| | | YLTLNDGSQAVGRSSFYCLEYFPSQMLRTGNNFQFSYEFENVPFHSSYAHSQSL |
| | | DRLMNPLIDQYLYYLSKTINGSGQNQQTLKFSVAGPSNMAVQGRNYIPGPSYRQ |
| | | QRVSTTVTQNNNSEFAWPGASSWALNGRNSLMNPGPAMASHKEGEDRFFPLSGS |
| | | LIFGKQGTGRDNVDADKVMITNEEEIKTTNPVATESYGQVATNHQSAQAYGLPK |
| | | GPQAQTGWVQNQGILPGMVWQDRDVYLQGPIWAKIPHTDGNFHPSPLMGGFGMK |
| | | HPPPQILIKNTPVPADPPTAFNKDKLNSFITQYSTGQVSVEIEWELQKENSKRW |
| | | NPEIQYTSNYYKSNNVEFAVNTEGVYSEPRPIGTRYLTRNL |
| TTD-009 | 3644 | MAADGYLPDWLEDNLSEGIREWWALKPGAPQPKANQQHQDNARGLVLPGYKYLG |
| 7 mer peptide | | PGNGLDKGEPVNAADAAALEHDKAYDQQLKAGDNPYLKYNHADAEFQERLKEDT |
| underlined, starts at | | SFGGNLGRAVFQAKKRLLEPLGLVEEAAKTAPGKKRPVEQSPQEPDSSAGIGKS |
| position 590 | | GAQPAKKRLNFGQTGDTESVPDPQPIGEPPAAPSGVGSLTMASGGGAPVADNNE |
| (immediately | | GADGVGSSSGNWHCDSQWLGDRVITTSTRTWALPTYNNHLYKQISNSTSGGSSN |
| subsequent to 589); | | DNAYFGYSTPWGYFDFNRFHCHFSPRDWQRLINNNWGFRPKRLNFKLFNIQVKE |
| 743 aa | | VTDNNGVKTIANNLTSTVQVFTDSDYQLPYVLGSAHEGCLPPFPADVFMIPQYG |
| | | YLTLNDGSQAVGRSSFYCLEYFPSQMLRTGNNFQFSYEFENVPFHSSYAHSQSL |
| | | DRLMNPLIDQYLYYLSKTINGSGQNQQTLKFSVAGPSNMAVQGRNYIPGPSYRQ |
| | | QRVSTTVTQNNNSEFAWPGASSWALNGRNSLMNPGPAMASHKEGEDRFFPLSGS |
| | | LIFGKQGTGRDNVDADKVMITNEEEIKTTNPVATESYGQVATNHQSAQASTGTL |
| | | RLQAQTGWVQNQGILPGMVWQDRDVYLQGPIWAKIPHTDGNFHPSPLMGGFGMK |
| | | HPPPQILIKNTPVPADPPTAFNKDKLNSFITQYSTGQVSVEIEWELQKENSKRW |
| | | NPEIQYTSNYYKSNNVEFAVNTEGVYSEPRPIGTRYLTRNL |
| TTD-010 | 3645 | MAADGYLPDWLEDNLSEGIREWWALKPGAPQPKANQQHQDNARGLVLPGYKYLG |
| 7 mer peptide | | PGNGLDKGEPVNAADAAALEHDKAYDQQLKAGDNPYLKYNHADAEFQERLKEDT |
| underlined, starts at | | SFGGNLGRAVFQAKKRLLEPLGLVEEAAKTAPGKKRPVEQSPQEPDSSAGIGKS |
| position 590 | | GAQPAKKRLNFGQTGDTESVPDPQPIGEPPAAPSGVGSLTMASGGGAPVADNNE |
| (immediately | | GADGVGSSSGNWHCDSQWLGDRVITTSTRTWALPTYNNHLYKQISNSTSGGSSN |
| subsequent to 589); | | DNAYFGYSTPWGYFDFNRFHCHFSPRDWQRLINNNWGFRPKRLNFKLFNIQVKE |
| 743 aa | | VTDNNGVKTIANNLTSTVQVFTDSDYQLPYVLGSAHEGCLPPFPADVEMIPQYG |
| | | YLTLNDGSQAVGRSSFYCLEYFPSQMLRTGNNFQFSYEFENVPFHSSYAHSQSL |
| | | DRLMNPLIDQYLYYLSKTINGSGQNQQTLKFSVAGPSNMAVQGRNYIPGPSYRQ |
| | | QRVSTTVTQNNNSEFAWPGASSWALNGRNSLMNPGPAMASHKEGEDRFFPLSGS |
| | | LIFGKQGTGRDNVDADKVMITNEEEIKTTNPVATESYGQVATNHQSAQAYSTDE |
| | | RMQAQTGWVQNQGILPGMVWQDRDVYLQGPIWAKIPHTDGNFHPSPLMGGFGMK |
| | | HPPPQILIKNTPVPADPPTAFNKDKLNSFITQYSTGQVSVEIEWELQKENSKRW |
| | | NPEIQYTSNYYKSNNVEFAVNTEGVYSEPRPIGTRYLTRNL |
| TTD-011 | 3646 | MAADGYLPDWLEDNLSEGIREWWALKPGAPQPKANQQHQDNARGLVLPGYKYLG |
| 7 mer peptide | | PGNGLDKGEPVNAADAAALEHDKAYDQQLKAGDNPYLKYNHADAEFQERLKEDT |
| underlined, starts at | | SFGGNLGRAVFQAKKRLLEPLGLVEEAAKTAPGKKRPVEQSPQEPDSSAGIGKS |
| position 590 | | GAQPAKKRLNFGQTGDTESVPDPQPIGEPPAAPSGVGSLTMASGGGAPVADNNE |
| (immediately | | GADGVGSSSGNWHCDSQWLGDRVITTSTRTWALPTYNNHLYKQISNSTSGGSSN |
| subsequent to 589); | | DNAYFGYSTPWGYFDFNRFHCHFSPRDWQRLINNNWGFRPKRLNFKLFNIQVKE |
| 743 aa | | VTDNNGVKTIANNLTSTVQVFTDSDYQLPYVLGSAHEGCLPPFPADVEMIPQYG |
| | | YLTLNDGSQAVGRSSFYCLEYFPSQMLRTGNNFQFSYEFENVPFHSSYAHSQSL |
| | | DRLMNPLIDQYLYYLSKTINGSGQNQQTLKFSVAGPSNMAVQGRNYIPGPSYRQ |
| | | QRVSTTVTQNNNSEFAWPGASSWALNGRNSLMNPGPAMASHKEGEDRFFPLSGS |
| | | LIFGKQGTGRDNVDADKVMITNEEEIKTTNPVATESYGQVATNHQSAQAYSTDE |
| | | RKQAQTGWVQNQGILPGMVWQDRDVYLQGPIWAKIPHTDGNFHPSPLMGGFGMK |
| | | HPPPQILIKNTPVPADPPTAFNKDKLNSFITQYSTGQVSVEIEWELQKENSKRW |
| | | NPEIQYTSNYYKSNNVEFAVNTEGVYSEPRPIGTRYLTRNL |
| TTD-012 | 3647 | MAADGYLPDWLEDNLSEGIREWWALKPGAPQPKANQQHQDNARGLVLPGYKYLG |
| 7 mer peptide | | PGNGLDKGEPVNAADAAALEHDKAYDQQLKAGDNPYLKYNHADAEFQERLKEDT |
| underlined, starts at | | SFGGNLGRAVFQAKKRLLEPLGLVEEAAKTAPGKKRPVEQSPQEPDSSAGIGKS |
| position 590 | | GAQPAKKRLNFGQTGDTESVPDPQPIGEPPAAPSGVGSLTMASGGGAPVADNNE |
| (immediately | | GADGVGSSSGNWHCDSQWLGDRVITTSTRTWALPTYNNHLYKQISNSTSGGSSN |
| subsequent to 589); | | DNAYFGYSTPWGYFDFNRFHCHFSPRDWQRLINNNWGFRPKRLNFKLFNIQVKE |
| 743 aa | | VTDNNGVKTIANNLTSTVQVFTDSDYQLPYVLGSAHEGCLPPFPADVEMIPQYG |
| | | YLTLNDGSQAVGRSSFYCLEYFPSQMLRTGNNFQFSYEFENVPFHSSYAHSQSL |
| | | DRLMNPLIDQYLYYLSKTINGSGQNQQTLKFSVAGPSNMAVQGRNYIPGPSYRQ |
| | | QRVSTTVTQNNNSEFAWPGASSWALNGRNSLMNPGPAMASHKEGEDRFFPLSGS |
| | | LIFGKQGTGRDNVDADKVMITNEEEIKTTNPVATESYGQVATNHQSAQAYVSSV |
| | | KMQAQTGWVQNQGILPGMVWQDRDVYLQGPIWAKIPHTDGNFHPSPLMGGFGMK |
| | | HPPPQILIKNTPVPADPPTAFNKDKLNSFITQYSTGQVSVEIEWELQKENSKRW |
| | | NPEIQYTSNYYKSNNVEFAVNTEGVYSEPRPIGTRYLTRNL |

TABLE 9
Exemplary full length capsid nucleic acid sequences

| Name and | SEQ ID | |
|---|---|---|
| Annotation | NO: | NT Sequence |
| TTD-001 | 3623 | atggctgccgatggttatcttccagattggctcgaggacaaccttagtgaaggaattcgcg |
| 9 mer peptide | | agtggtgggctttgaaacctggagcccctcaacccaaggcaaatcaacaacatcaagacaa |
| underlined | | cgctcgaggtcttgtgcttccgggttacaaataccttggacccggcaacggactcgacaag |
| | | ggggagccggtcaacgcagcagacgcggcggccctcgagcacgacaaggcctacgaccagc |
| | | agctcaaggccggagacaacccgtacctcaagtacaaccacgccgacgccgagttccagga |
| | | gcggctcaaagaagatacgtcttttgggggcaacctcgggcgagcagtcttccaggccaaa |
| | | aagaggcttcttgaacctcttggtctggttgaggaagcggctaagacggctcctggaaaga |
| | | agaggcctgtagagcagtctcctcaggaaccggactcctccgcgggtattggcaaatcggg |
| | | tgcacagcccgctaaaaagagactcaatttcggtcagactggcgacacagagtcagtccca |
| | | gaccctcaaccaatcggagaacctcccgcagccccctcaggtgtgggatctcttacaatgg |
| | | cttcaggtggtggcgcaccagtggcagacaataacgaaggtgccgatggagtgggtagttc |
| | | ctcgggaaattggcattgcgattcccaatggctgggggacagagtcatcaccaccagcacc |
| | | cgaacctgggccctgcccacctacaacaatcacctctacaagcaaatctccaacagcacat |
| | | ctggaggatcttcaaatgacaacgcctacttcggctacagcaccccctgggggtattttga |
| | | cttcaacagattccactgccacttctcaccacgtgactggcagcgactcatcaacaacaac |
| | | tggggattccggcctaagcgactcaacttcaagctcttcaacattcaggtcaaagaggtta |
| | | cggacaacaatggagtcaagaccatcgccaataaccttaccagcacggtccaggtcttcac |
| | | ggactcagactatcagctcccgtacgtgctcgggtcggctcacgagggctgcctcccgccg |
| | | ttcccagcggacgttttcatgattcctcagtacgggtatctgacgcttaatgatggaagcc |
| | | aggccgtgggtcgttcgtccttttactgcctggaatatttcccgtcgcaaatgctaagaac |
| | | gggtaacaacttccagttcagctacgagtttgagaacgtacctttccatagcagctacgct |
| | | cacagccaaagcctggaccgactaatgaatccactcatcgaccaatacttgtactatctct |
| | | caaagactattaacggttctggacagaatcaacaaacgctaaaattcagtgtggccggacc |
| | | cagcaacatggctgtccagggaagaaactacatacctggacccagctaccgacaacaacgt |
| | | gtctcaaccactgtgactcaaaacaacaacagcgaatttgcttggcctggagcttcttctt |
| | | gggctctcaatggacgtaatagcttgatgaatcctggacctgctatggccagccacaaaga |
| | | aggagaggaccgtttctttcctttgtctggatctttaatttttggcaaacaaggaactgga |
| | | agagacaacgtggatgcggacaaagtcatgataaccaacgaagaagaaattaaaactacta |
| | | acccggtagcaacggagtcctatggacaagtggccacaaaccaccagagtccgcttaatgg |
| | | tgccgtccatctttatgctcaggcgcagaccggctgggttcaaaaccaaggaatacttccg |
| | | ggtatggtttggcaggacagagatgtgtacctgcaaggacccatttgggccaaaattcctc |
| | | acacggacggcaactttcacccttctccgctgatgggagggtttggaatgaagcacccgcc |
| | | tcctcagatcctcatcaaaaacacacctgtacctgcCgatcctccaacggccttcaacaag |
| | | gacaagctgaactctttcatcacccagtattctactggccaagtcagcgtggagatcgagt |
| | | gggagctgcagaaggaaaacagcaagcgGtggaacccggagatccagtacacttccaacta |
| | | ttacaagtctaataatgttgaatttgctgttaatactgaaggtgtatatagtgaaccccgc |
| | | cccattggcaccagatacctgactcgtaatctgtaa |
| TTD-002 | 3624 | atggctgccgatggttatcttccagattggctcgaggacaaccttagtgaaggaattcgcg |
| 7 mer peptide | | agtggtgggctttgaaacctggagcccctcaacccaaggcaaatcaacaacatcaagacaa |
| underlined | | cgctcgaggtcttgtgcttccgggttacaaataccttggacccggcaacggactcgacaag |
| | | ggggagccggtcaacgcagcagacgcggcggccctcgagcacgacaaggcctacgaccagc |
| | | agctcaaggccggagacaacccgtacctcaagtacaaccacgccgacgccgagttccagga |
| | | gcggctcaaagaagatacgtcttttgggggcaacctcgggcgagcagtcttccaggccaaa |
| | | aagaggcttcttgaacctcttggtctggttgaggaagcggctaagacggctcctggaaaga |
| | | agaggcctgtagagcagtctcctcaggaaccggactcctccgcgggtattggcaaatcggg |
| | | tgcacagcccgctaaaaagagactcaatttcggtcagactggcgacacagagtcagtccca |
| | | gaccctcaaccaatcggagaacctcccgcagccccctcaggtgtgggatctcttacaatgg |
| | | cttcaggtggtggcgcaccagtggcagacaataacgaaggtgccgatggagtgggtagttc |
| | | ctcgggaaattggcattgcgattcccaatggctgggggacagagtcatcaccaccagcacc |
| | | cgaacctgggccctgcccacctacaacaatcacctctacaagcaaatctccaacagcacat |
| | | ctggaggatcttcaaatgacaacgcctacttcggctacagcaccccctgggggtattttga |
| | | cttcaacagattccactgccacttctcaccacgtgactggcagcgactcatcaacaacaac |
| | | tggggattccggcctaagcgactcaacttcaagctcttcaacattcaggtcaaagaggtta |
| | | cggacaacaatggagtcaagaccatcgccaataaccttaccagcacggtccaggtcttcac |
| | | ggactcagactatcagctcccgtacgtgctcgggtcggctcacgagggctgcctcccgccg |
| | | ttcccagcggacgttttcatgattcctcagtacgggtatctgacgcttaatgatggaagcc |
| | | aggccgtgggtcgttcgtccttttactgcctggaatatttcccgtcgcaaatgctaagaac |
| | | gggtaacaacttccagttcagctacgagtttgagaacgtacctttccatagcagctacgct |
| | | cacagccaaagcctggaccgactaatgaatccactcatcgaccaatacttgtactatctct |
| | | caaagactattaacggttctggacagaatcaacaaacgctaaaattcagtgtggccggacc |
| | | cagcaacatggctgtccagggaagaaactacatacctggacccagctaccgacaacaacgt |
| | | gtctcaaccactgtgactcaaaacaacaacagcgaatttgcttggcctggagcttcttctt |
| | | gggctctcaatggacgtaatagcttgatgaatcctggacctgctatggccagccacaaaga |
| | | aggagaggaccgtttctttcctttgtctggatctttaatttttggcaaacaaggaactgga |
| | | agagacaacgtggatgcggacaaagtcatgataaccaacgaagaagaaattaaaactacta |
| | | acccggtagcaacggagtcctatggacaagtggccacaaaccaccagagtgcacaggctcg |
| | | tgattctccgaagggttggcaggcgcagaccggctgggttcaaaaccaaggaatacttccg |
| | | ggtatggtttggcaggacagagatgtgtacctgcaaggacccatttgggccaaaattcctc |
| | | acacggacggcaactttcacccttctccgctgatgggagggtttggaatgaagcacccgcc |
| | | tcctcagatcctcatcaaaaacacacctgtacctgcCgatcctccaacggccttcaacaag |
| | | gacaagctgaactctttcatcacccagtattctactggccaagtcagcgtggagatcgagt |
| | | gggagctgcagaaggaaaacagcaagcgGtggaacccggagatccagtacacttccaacta |
| | | ttacaagtctaataatgttgaatttgctgttaatactgaaggtgtatatagtgaaccccgc |
| | | cccattggcaccagatacctgactcgtaatctgtaa |
| | 3625 | atggctgccgatggttatcttccagattggctcgaggacaaccttagtgaaggaattcgcg |
| | | agtggtgggctttgaaacctggagcccctcaacccaaggcaaatcaacaacatcaagacaa |
| | | cgctcgaggtcttgtgcttccgggttacaaataccttggacccggcaacggactcgacaag |
| | | ggggagccggtcaacgcagcagacgcggcggccctcgagcacgacaaggcctacgaccagc |
| | | agctcaaggccggagacaacccgtacctcaagtacaaccacgccgacgccgagttccagga |
| | | gcggctcaaagaagatacgtcttttgggggcaacctcgggcgagcagtcttccaggccaaa |
| | | aagaggcttcttgaacctcttggtctggttgaggaagcggctaagacggctcctggaaaga |
| | | agaggcctgtagagcagtctcctcaggaaccggactcctccgcgggtattggcaaatcggg |
| | | tgcacagcccgctaaaaagagactcaatttcggtcagactggcgacacagagtcagtccca |
| | | gaccctcaaccaatcggagaacctcccgcagccccctcaggtgtgggatctcttacaatgg |
| | | cttcaggtggtggcgcaccagtggcagacaataacgaaggtgccgatggagtgggtagttc |
| | | ctcgggaaattggcattgcgattcccaatggctgggggacagagtcatcaccaccagcacc |
| | | cgaacctgggccctgcccacctacaacaatcacctctacaagcaaatctccaacagcacat |
| | | ctggaggatcttcaaatgacaacgcctacttcggctacagcaccccctgggggtattttga |
| | | cttcaacagattccactgccacttctcaccacgtgactggcagcgactcatcaacaacaac |
| | | tggggattccggcctaagcgactcaacttcaagctcttcaacattcaggtcaaagaggtta |
| | | cggacaacaatggagtcaagaccatcgccaataaccttaccagcacggtccaggtcttcac |
| | | ggactcagactatcagctcccgtacgtgctcgggtcggctcacgagggctgcctcccgccg |
| | | ttcccagcggacgttttcatgattcctcagtacgggtatctgacgcttaatgatggaagcc |
| | | aggccgtgggtcgttcgtccttttactgcctggaatatttcccgtcgcaaatgctaagaac |
| | | gggtaacaacttccagttcagctacgagtttgagaacgtacctttccatagcagctacgct |
| | | cacagccaaagcctggaccgactaatgaatccactcatcgaccaatacttgtactatctct |
| | | caaagactattaacggttctggacagaatcaacaaacgctaaaattcagtgtggccggacc |
| | | cagcaacatggctgtccagggaagaaactacatacctggacccagctaccgacaacaacgt |
| | | gtctcaaccactgtgactcaaaacaacaacagcgaatttgcttggcctggagcttcttctt |
| | | gggctctcaatggacgtaatagcttgatgaatcctggacctgctatggccagccacaaaga |
| | | aggagaggaccgtttctttcctttgtctggatctttaatttttggcaaacaaggaactgga |
| | | agagacaacgtggatgcggacaaagtcatgataaccaacgaagaagaaattaaaactacta |
| | | acccggtagcaacggagtcctatggacaagtggccacaaaccaccagagtgcacaggctcg |
| | | tgattctccgaagggttggcaggcgcagaccggctgggttcaaaaccaaggaatacttccg |
| | | ggtatggtttggcaggacagagatgtgtacctgcaaggacccatttgggccaaaattcctc |
| | | acacggacggcaactttcacccttctccgctgatgggagggtttggaatgaagcacccgcc |
| | | tcctcagatcctcatcaaaaacacacctgtacctgcggatcctccaacggccttcaacaag |
| | | gacaagctgaactctttcatcacccagtattctactggccaagtcagcgtggagatcgagt |
| | | gggagctgcagaaggaaaacagcaagcgctggaacccggagatccagtacacttccaacta |
| | | ttacaagtctaataatgttgaatttgctgttaatactgaaggtgtatatagtgaaccccgc |
| | | cccattggcaccagatacctgactcgtaatctgtaa |
| TTD-003 | 3626 | atggctgccgatggttatcttccagattggctcgaggacaaccttagtgaaggaattcgcg |
| 7 mer peptide | | agtggtgggctttgaaacctggagcccctcaacccaaggcaaatcaacaacatcaagacaa |
| underlined | | cgctcgaggtcttgtgcttccgggttacaaataccttggacccggcaacggactcgacaag |
| | | ggggagccggtcaacgcagcagacgcggcggccctcgagcacgacaaggcctacgaccagc |
| | | agctcaaggccggagacaacccgtacctcaagtacaaccacgccgacgccgagttccagga |
| | | gcggctcaaagaagatacgtcttttgggggcaacctcgggcgagcagtcttccaggccaaa |
| | | aagaggcttcttgaacctcttggtctggttgaggaagcggctaagacggctcctggaaaga |
| | | agaggcctgtagagcagtctcctcaggaaccggactcctccgcgggtattggcaaatcggg |
| | | tgcacagcccgctaaaaagagactcaatttcggtcagactggcgacacagagtcagtccca |
| | | gaccctcaaccaatcggagaacctcccgcagccccctcaggtgtgggatctcttacaatgg |
| | | cttcaggtggtggcgcaccagtggcagacaataacgaaggtgccgatggagtgggtagttc |
| | | ctcgggaaattggcattgcgattcccaatggctgggggacagagtcatcaccaccagcacc |
| | | cgaacctgggccctgcccacctacaacaatcacctctacaagcaaatctccaacagcacat |
| | | ctggaggatcttcaaatgacaacgcctacttcggctacagcaccccctgggggtattttga |
| | | cttcaacagattccactgccacttctcaccacgtgactggcagcgactcatcaacaacaac |
| | | tggggattccggcctaagcgactcaacttcaagctcttcaacattcaggtcaaagaggtta |
| | | cggacaacaatggagtcaagaccatcgccaataaccttaccagcacggtccaggtcttcac |
| | | ggactcagactatcagctcccgtacgtgctcgggtcggctcacgagggctgcctcccgccg |
| | | ttcccagcggacgttttcatgattcctcagtacgggtatctgacgcttaatgatggaagcc |
| | | aggccgtgggtcgttcgtccttttactgcctggaatatttcccgtcgcaaatgctaagaac |
| | | gggtaacaacttccagttcagctacgagtttgagaacgtacctttccatagcagctacgct |
| | | cacagccaaagcctggaccgactaatgaatccactcatcgaccaatacttgtactatctct |
| | | caaagactattaacggttctggacagaatcaacaaacgctaaaattcagtgtggccggacc |
| | | cagcaacatggctgtccagggaagaaactacatacctggacccagctaccgacaacaacgt |
| | | gtctcaaccactgtgactcaaaacaacaacagcgaatttgcttggcctggagcttcttctt |
| | | gggctctcaatggacgtaatagcttgatgaatcctggacctgctatggccagccacaaaga |
| | | aggagaggaccgtttctttcctttgtctggatctttaatttttggcaaacaaggaactgga |
| | | agagacaacgtggatgcggacaaagtcatgataaccaacgaagaagaaattaaaactacta |
| | | acccggtagcaacggagtcctatggacaagtggccacaaaccaccagagtgcacaggctta |
| | | ttctacggatgtgaggatgcaggcgcagaccggctgggttcaaaaccaaggaatacttccg |
| | | ggtatggtttggcaggacagagatgtgtacctgcaaggacccatttgggccaaaattcctc |
| | | acacggacggcaactttcacccttctccgctgatgggagggtttggaatgaagcacccgcc |
| | | tcctcagatcctcatcaaaaacacacctgtacctgcCgatcctccaacggccttcaacaag |
| | | gacaagctgaactctttcatcacccagtattctactggccaagtcagcgtggagatcgagt |
| | | gggagctgcagaaggaaaacagcaagcgGtggaacccggagatccagtacacttccaacta |
| | | ttacaagtctaataatgttgaatttgctgttaatactgaaggtgtatatagtgaaccccgc |
| | | cccattggcaccagatacctgactcgtaatctgtaa |
| TTD-004 | 3627 | atggctgccgatggttatcttccagattggctcgaggacaaccttagtgaaggaattcgcg |
| 7 mer peptide | | agtggtgggctttgaaacctggagcccctcaacccaaggcaaatcaacaacatcaagacaa |
| underlined | | cgctcgaggtcttgtgcttccgggttacaaataccttggacccggcaacggactcgacaag |
| | | ggggagccggtcaacgcagcagacgcggcggccctcgagcacgacaaggcctacgaccagc |
| | | agctcaaggccggagacaacccgtacctcaagtacaaccacgccgacgccgagttccagga |
| | | gcggctcaaagaagatacgtcttttgggggcaacctcgggcgagcagtcttccaggccaaa |
| | | aagaggcttcttgaacctcttggtctggttgaggaagcggctaagacggctcctggaaaga |
| | | agaggcctgtagagcagtctcctcaggaaccggactcctccgcgggtattggcaaatcggg |
| | | tgcacagcccgctaaaaagagactcaatttcggtcagactggcgacacagagtcagtccca |
| | | gaccctcaaccaatcggagaacctcccgcagccccctcaggtgtgggatctcttacaatgg |
| | | cttcaggtggtggcgcaccagtggcagacaataacgaaggtgccgatggagtgggtagttc |
| | | ctcgggaaattggcattgcgattcccaatggctgggggacagagtcatcaccaccagcacc |
| | | cgaacctgggccctgcccacctacaacaatcacctctacaagcaaatctccaacagcacat |
| | | ctggaggatcttcaaatgacaacgcctacttcggctacagcaccccctgggggtattttga |
| | | cttcaacagattccactgccacttctcaccacgtgactggcagcgactcatcaacaacaac |
| | | tggggattccggcctaagcgactcaacttcaagctcttcaacattcaggtcaaagaggtta |
| | | cggacaacaatggagtcaagaccatcgccaataaccttaccagcacggtccaggtcttcac |
| | | ggactcagactatcagctcccgtacgtgctcgggtcggctcacgagggctgcctcccgccg |
| | | ttcccagcggacgttttcatgattcctcagtacgggtatctgacgcttaatgatggaagcc |
| | | aggccgtgggtcgttcgtccttttactgcctggaatatttcccgtcgcaaatgctaagaac |
| | | gggtaacaacttccagttcagctacgagtttgagaacgtacctttccatagcagctacgct |
| | | cacagccaaagcctggaccgactaatgaatccactcatcgaccaatacttgtactatctct |
| | | caaagactattaacggttctggacagaatcaacaaacgctaaaattcagtgtggccggacc |
| | | cagcaacatggctgtccagggaagaaactacatacctggacccagctaccgacaacaacgt |
| | | gtctcaaccactgtgactcaaaacaacaacagcgaatttgcttggcctggagcttcttctt |
| | | gggctctcaatggacgtaatagcttgatgaatcctggacctgctatggccagccacaaaga |
| | | aggagaggaccgtttctttcctttgtctggatctttaatttttggcaaacaaggaactgga |
| | | agagacaacgtggatgcggacaaagtcatgataaccaacgaagaagaaattaaaactacta |
| | | acccggtagcaacggagtcctatggacaagtggccacaaaccaccagagtgcacagattgt |
| | | tatgaattcgttgaaggctcaggcgcagaccggctgggttcaaaaccaaggaatacttccg |
| | | ggtatggtttggcaggacagagatgtgtacctgcaaggacccatttgggccaaaattcctc |
| | | acacggacggcaactttcacccttctccgctgatgggagggtttggaatgaagcacccgcc |
| | | tcctcagatcctcatcaaaaacacacctgtacctgcCgatcctccaacggccttcaacaag |
| | | gacaagctgaactctttcatcacccagtattctactggccaagtcagcgtggagatcgagt |
| | | gggagctgcagaaggaaaacagcaagcgGtggaacccggagatccagtacacttccaacta |
| | | ttacaagtctaataatgttgaatttgctgttaatactgaaggtgtatatagtgaaccccgc |
| | | cccattggcaccagatacctgactcgtaatctgtaa |
| TTD-005 | 3628 | atggctgccgatggttatcttccagattggctcgaggacaaccttagtgaaggaattcgcg |
| 7 mer peptide | | agtggtgggctttgaaacctggagcccctcaacccaaggcaaatcaacaacatcaagacaa |
| underlined | | cgctcgaggtcttgtgcttccgggttacaaataccttggacccggcaacggactcgacaag |
| | | ggggagccggtcaacgcagcagacgcggcggccctcgagcacgacaaggcctacgaccagc |
| | | agctcaaggccggagacaacccgtacctcaagtacaaccacgccgacgccgagttccagga |
| | | gcggctcaaagaagatacgtcttttgggggcaacctcgggcgagcagtcttccaggccaaa |
| | | aagaggcttcttgaacctcttggtctggttgaggaagcggctaagacggctcctggaaaga |
| | | agaggcctgtagagcagtctcctcaggaaccggactcctccgcgggtattggcaaatcggg |
| | | tgcacagcccgctaaaaagagactcaatttcggtcagactggcgacacagagtcagtccca |
| | | gaccctcaaccaatcggagaacctcccgcagccccctcaggtgtgggatctcttacaatgg |
| | | cttcaggtggtggcgcaccagtggcagacaataacgaaggtgccgatggagtgggtagttc |
| | | ctcgggaaattggcattgcgattcccaatggctgggggacagagtcatcaccaccagcacc |
| | | cgaacctgggccctgcccacctacaacaatcacctctacaagcaaatctccaacagcacat |
| | | ctggaggatcttcaaatgacaacgcctacttcggctacagcaccccctgggggtattttga |
| | | cttcaacagattccactgccacttctcaccacgtgactggcagcgactcatcaacaacaac |
| | | tggggattccggcctaagcgactcaacttcaagctcttcaacattcaggtcaaagaggtta |
| | | cggacaacaatggagtcaagaccatcgccaataaccttaccagcacggtccaggtcttcac |
| | | ggactcagactatcagctcccgtacgtgctcgggtcggctcacgagggctgcctcccgccg |
| | | ttcccagcggacgttttcatgattcctcagtacgggtatctgacgcttaatgatggaagcc |
| | | aggccgtgggtcgttcgtccttttactgcctggaatatttcccgtcgcaaatgctaagaac |
| | | gggtaacaacttccagttcagctacgagtttgagaacgtacctttccatagcagctacgct |
| | | cacagccaaagcctggaccgactaatgaatccactcatcgaccaatacttgtactatctct |
| | | caaagactattaacggttctggacagaatcaacaaacgctaaaattcagtgtggccggacc |
| | | cagcaacatggctgtccagggaagaaactacatacctggacccagctaccgacaacaacgt |
| | | gtctcaaccactgtgactcaaaacaacaacagcgaatttgcttggcctggagcttcttctt |
| | | gggctctcaatggacgtaatagcttgatgaatcctggacctgctatggccagccacaaaga |
| | | aggagaggaccgtttctttcctttgtctggatctttaatttttggcaaacaaggaactgga |
| | | agagacaacgtggatgcggacaaagtcatgataaccaacgaagaagaaattaaaactacta |
| | | acccggtagcaacggagtcctatggacaagtggccacaaaccaccagagtgcacaggctcg |
| | | ggagagtcctcgtgggctgcaggcgcagaccggctgggttcaaaaccaaggaatacttccg |
| | | ggtatggtttggcaggacagagatgtgtacctgcaaggacccatttgggccaaaattcctc |
| | | acacggacggcaactttcacccttctccgctgatgggagggtttggaatgaagcacccgcc |
| | | tcctcagatcctcatcaaaaacacacctgtacctgcCgatcctccaacggccttcaacaag |
| | | gacaagctgaactctttcatcacccagtattctactggccaagtcagcgtggagatcgagt |
| | | gggagctgcagaaggaaaacagcaagcgGtggaacccggagatccagtacacttccaacta |
| | | ttacaagtctaataatgttgaatttgctgttaatactgaaggtgtatatagtgaaccccgc |
| | | cccattggcaccagatacctgactcgtaatctgtaa |
| TTD-006 | 3629 | atggctgccgatggttatcttccagattggctcgaggacaaccttagtgaaggaattcgcg |
| 7 mer peptide | | agtggtgggctttgaaacctggagcccctcaacccaaggcaaatcaacaacatcaagacaa |
| underlined | | cgctcgaggtcttgtgcttccgggttacaaataccttggacccggcaacggactcgacaag |
| | | ggggagccggtcaacgcagcagacgcggcggccctcgagcacgacaaggcctacgaccagc |
| | | agctcaaggccggagacaacccgtacctcaagtacaaccacgccgacgccgagttccagga |
| | | gcggctcaaagaagatacgtcttttgggggcaacctcgggcgagcagtcttccaggccaaa |
| | | aagaggcttcttgaacctcttggtctggttgaggaagcggctaagacggctcctggaaaga |
| | | agaggcctgtagagcagtctcctcaggaaccggactcctccgcgggtattggcaaatcggg |
| | | tgcacagcccgctaaaaagagactcaatttcggtcagactggcgacacagagtcagtccca |
| | | gaccctcaaccaatcggagaacctcccgcagccccctcaggtgtgggatctcttacaatgg |
| | | cttcaggtggtggcgcaccagtggcagacaataacgaaggtgccgatggagtgggtagttc |
| | | ctcgggaaattggcattgcgattcccaatggctgggggacagagtcatcaccaccagcacc |
| | | cgaacctgggccctgcccacctacaacaatcacctctacaagcaaatctccaacagcacat |
| | | ctggaggatcttcaaatgacaacgcctacttcggctacagcaccccctgggggtattttga |
| | | cttcaacagattccactgccacttctcaccacgtgactggcagcgactcatcaacaacaac |
| | | tggggattccggcctaagcgactcaacttcaagctcttcaacattcaggtcaaagaggtta |
| | | cggacaacaatggagtcaagaccatcgccaataaccttaccagcacggtccaggtcttcac |
| | | ggactcagactatcagctcccgtacgtgctcgggtcggctcacgagggctgcctcccgccg |
| | | ttcccagcggacgttttcatgattcctcagtacgggtatctgacgcttaatgatggaagcc |
| | | aggccgtgggtcgttcgtccttttactgcctggaatatttcccgtcgcaaatgctaagaac |
| | | gggtaacaacttccagttcagctacgagtttgagaacgtacctttccatagcagctacgct |
| | | cacagccaaagcctggaccgactaatgaatccactcatcgaccaatacttgtactatctct |
| | | caaagactattaacggttctggacagaatcaacaaacgctaaaattcagtgtggccggacc |
| | | cagcaacatggctgtccagggaagaaactacatacctggacccagctaccgacaacaacgt |
| | | gtctcaaccactgtgactcaaaacaacaacagcgaatttgcttggcctggagcttcttctt |
| | | gggctctcaatggacgtaatagcttgatgaatcctggacctgctatggccagccacaaaga |
| | | aggagaggaccgtttctttcctttgtctggatctttaatttttggcaaacaaggaactgga |
| | | agagacaacgtggatgcggacaaagtcatgataaccaacgaagaagaaattaaaactacta |
| | | acccggtagcaacggagtcctatggacaagtggccacaaaccaccagagtgcacaggctag |
| | | ttttaatgatactagggctcaggcgcagaccggctgggttcaaaaccaaggaatacttccg |
| | | ggtatggtttggcaggacagagatgtgtacctgcaaggacccatttgggccaaaattcctc |
| | | acacggacggcaactttcacccttctccgctgatgggagggtttggaatgaagcacccgcc |
| | | tcctcagatcctcatcaaaaacacacctgtacctgcCgatcctccaacggccttcaacaag |
| | | gacaagctgaactctttcatcacccagtattctactggccaagtcagcgtggagatcgagt |
| | | gggagctgcagaaggaaaacagcaagcgGtggaacccggagatccagtacacttccaacta |
| | | ttacaagtctaataatgttgaatttgctgttaatactgaaggtgtatatagtgaaccccgc |
| | | cccattggcaccagatacctgactcgtaatctgtaa |
| TTD-007 | 3630 | atggctgccgatggttatcttccagattggctcgaggacaaccttagtgaaggaattcgcg |
| 9 mer peptide | | agtggtgggctttgaaacctggagcccctcaacccaaggcaaatcaacaacatcaagacaa |
| underlined | | cgctcgaggtcttgtgcttccgggttacaaataccttggacccggcaacggactcgacaag |
| | | ggggagccggtcaacgcagcagacgcggcggccctcgagcacgacaaggcctacgaccagc |
| | | agctcaaggccggagacaacccgtacctcaagtacaaccacgccgacgccgagttccagga |
| | | gcggctcaaagaagatacgtcttttgggggcaacctcgggcgagcagtcttccaggccaaa |
| | | aagaggcttcttgaacctcttggtctggttgaggaagcggctaagacggctcctggaaaga |
| | | agaggcctgtagagcagtctcctcaggaaccggactcctccgcgggtattggcaaatcggg |
| | | tgcacagcccgctaaaaagagactcaatttcggtcagactggcgacacagagtcagtccca |
| | | gaccctcaaccaatcggagaacctcccgcagccccctcaggtgtgggatctcttacaatgg |
| | | cttcaggtggtggcgcaccagtggcagacaataacgaaggtgccgatggagtgggtagttc |
| | | ctcgggaaattggcattgcgattcccaatggctgggggacagagtcatcaccaccagcacc |
| | | cgaacctgggccctgcccacctacaacaatcacctctacaagcaaatctccaacagcacat |
| | | ctggaggatcttcaaatgacaacgcctacttcggctacagcaccccctgggggtattttga |
| | | cttcaacagattccactgccacttctcaccacgtgactggcagcgactcatcaacaacaac |
| | | tggggattccggcctaagcgactcaacttcaagctcttcaacattcaggtcaaagaggtta |
| | | cggacaacaatggagtcaagaccatcgccaataaccttaccagcacggtccaggtcttcac |
| | | ggactcagactatcagctcccgtacgtgctcgggtcggctcacgagggctgcctcccgccg |
| | | ttcccagcggacgttttcatgattcctcagtacgggtatctgacgcttaatgatggaagcc |
| | | aggccgtgggtcgttcgtccttttactgcctggaatatttcccgtcgcaaatgctaagaac |
| | | gggtaacaacttccagttcagctacgagtttgagaacgtacctttccatagcagctacgct |
| | | cacagccaaagcctggaccgactaatgaatccactcatcgaccaatacttgtactatctct |
| | | caaagactattaacggttctggacagaatcaacaaacgctaaaattcagtgtggccggacc |
| | | cagcaacatggctgtccagggaagaaactacatacctggacccagctaccgacaacaacgt |
| | | gtctcaaccactgtgactcaaaacaacaacagcgaatttgcttggcctggagcttcttctt |
| | | gggctctcaatggacgtaatagcttgatgaatcctggacctgctatggccagccacaaaga |
| | | aggagaggaccgtttctttcctttgtctggatctttaatttttggcaaacaaggaactgga |
| | | agagacaacgtggatgcggacaaagtcatgataaccaacgaagaagaaattaaaactacta |
| | | acccggtagcaacggagtcctatggacaagtggccacaaaccaccagagtggtggtacgtt |
| | | ggccgtcgtgtcgcttgctcaggcgcagaccggctgggttcaaaaccaaggaatacttccg |
| | | ggtatggtttggcaggacagagatgtgtacctgcaaggacccatttgggccaaaattcctc |
| | | acacggacggcaactttcacccttctccgctgatgggagggtttggaatgaagcacccgcc |
| | | tcctcagatcctcatcaaaaacacacctgtacctgcCgatcctccaacggccttcaacaag |
| | | gacaagctgaactctttcatcacccagtattctactggccaagtcagcgtggagatcgagt |
| | | gggagctgcagaaggaaaacagcaagcgGtggaacccggagatccagtacacttccaacta |
| | | ttacaagtctaataatgttgaatttgctgttaatactgaaggtgtatatagtgaaccccgc |
| | | cccattggcaccagatacctgactcgtaatctgtaa |
| TTD-008 | 3631 | atggctgccgatggttatcttccagattggctcgaggacaaccttagtgaaggaattcgcg |
| 7 mer peptide | | agtggtgggctttgaaacctggagcccctcaacccaaggcaaatcaacaacatcaagacaa |
| underlined | | cgctcgaggtcttgtgcttccgggttacaaataccttggacccggcaacggactcgacaag |
| | | ggggagccggtcaacgcagcagacgcggcggccctcgagcacgacaaggcctacgaccagc |
| | | agctcaaggccggagacaacccgtacctcaagtacaaccacgccgacgccgagttccagga |
| | | gcggctcaaagaagatacgtcttttgggggcaacctcgggcgagcagtcttccaggccaaa |
| | | aagaggcttcttgaacctcttggtctggttgaggaagcggctaagacggctcctggaaaga |
| | | agaggcctgtagagcagtctcctcaggaaccggactcctccgcgggtattggcaaatcggg |
| | | tgcacagcccgctaaaaagagactcaatttcggtcagactggcgacacagagtcagtccca |
| | | gaccctcaaccaatcggagaacctcccgcagccccctcaggtgtgggatctcttacaatgg |
| | | cttcaggtggtggcgcaccagtggcagacaataacgaaggtgccgatggagtgggtagttc |
| | | ctcgggaaattggcattgcgattcccaatggctgggggacagagtcatcaccaccagcacc |
| | | cgaacctgggccctgcccacctacaacaatcacctctacaagcaaatctccaacagcacat |
| | | ctggaggatcttcaaatgacaacgcctacttcggctacagcaccccctgggggtattttga |
| | | cttcaacagattccactgccacttctcaccacgtgactggcagcgactcatcaacaacaac |
| | | tggggattccggcctaagcgactcaacttcaagctcttcaacattcaggtcaaagaggtta |
| | | cggacaacaatggagtcaagaccatcgccaataaccttaccagcacggtccaggtcttcac |
| | | ggactcagactatcagctcccgtacgtgctcgggtcggctcacgagggctgcctcccgccg |
| | | ttcccagcggacgttttcatgattcctcagtacgggtatctgacgcttaatgatggaagcc |
| | | aggccgtgggtcgttcgtccttttactgcctggaatatttcccgtcgcaaatgctaagaac |
| | | gggtaacaacttccagttcagctacgagtttgagaacgtacctttccatagcagctacgct |
| | | cacagccaaagcctggaccgactaatgaatccactcatcgaccaatacttgtactatctct |
| | | caaagactattaacggttctggacagaatcaacaaacgctaaaattcagtgtggccggacc |
| | | cagcaacatggctgtccagggaagaaactacatacctggacccagctaccgacaacaacgt |
| | | gtctcaaccactgtgactcaaaacaacaacagcgaatttgcttggcctggagcttcttctt |
| | | gggctctcaatggacgtaatagcttgatgaatcctggacctgctatggccagccacaaaga |
| | | aggagaggaccgtttctttcctttgtctggatctttaatttttggcaaacaaggaactgga |
| | | agagacaacgtggatgcggacaaagtcatgataaccaacgaagaagaaattaaaactacta |
| | | acccggtagcaacggagtcctatggacaagtggccacaaaccaccagagtgcacaggctta |
| | | tgggttgccgaagggtcctcaggcgcagaccggctgggttcaaaaccaaggaatacttccg |
| | | ggtatggtttggcaggacagagatgtgtacctgcaaggacccatttgggccaaaattcctc |
| | | acacggacggcaactttcacccttctccgctgatgggagggtttggaatgaagcacccgcc |
| | | tcctcagatcctcatcaaaaacacacctgtacctgcCgatcctccaacggccttcaacaag |
| | | gacaagctgaactctttcatcacccagtattctactggccaagtcagcgtggagatcgagt |
| | | gggagctgcagaaggaaaacagcaagcgGtggaacccggagatccagtacacttccaacta |
| | | ttacaagtctaataatgttgaatttgctgttaatactgaaggtgtatatagtgaaccccgc |
| | | cccattggcaccagatacctgactcgtaatctgtaa |
| TTD-009 | 3632 | atggctgccgatggttatcttccagattggctcgaggacaaccttagtgaaggaattcgcg |
| 7 mer peptide | | agtggtgggctttgaaacctggagcccctcaacccaaggcaaatcaacaacatcaagacaa |
| underlined | | cgctcgaggtcttgtgcttccgggttacaaataccttggacccggcaacggactcgacaag |
| | | ggggagccggtcaacgcagcagacgcggcggccctcgagcacgacaaggcctacgaccagc |
| | | agctcaaggccggagacaacccgtacctcaagtacaaccacgccgacgccgagttccagga |
| | | gcggctcaaagaagatacgtcttttgggggcaacctcgggcgagcagtcttccaggccaaa |
| | | aagaggcttcttgaacctcttggtctggttgaggaagcggctaagacggctcctggaaaga |
| | | agaggcctgtagagcagtctcctcaggaaccggactcctccgcgggtattggcaaatcggg |
| | | tgcacagcccgctaaaaagagactcaatttcggtcagactggcgacacagagtcagtccca |
| | | gaccctcaaccaatcggagaacctcccgcagccccctcaggtgtgggatctcttacaatgg |
| | | cttcaggtggtggcgcaccagtggcagacaataacgaaggtgccgatggagtgggtagttc |
| | | ctcgggaaattggcattgcgattcccaatggctgggggacagagtcatcaccaccagcacc |
| | | cgaacctgggccctgcccacctacaacaatcacctctacaagcaaatctccaacagcacat |
| | | ctggaggatcttcaaatgacaacgcctacttcggctacagcaccccctgggggtattttga |
| | | cttcaacagattccactgccacttctcaccacgtgactggcagcgactcatcaacaacaac |
| | | tggggattccggcctaagcgactcaacttcaagctcttcaacattcaggtcaaagaggtta |
| | | cggacaacaatggagtcaagaccatcgccaataaccttaccagcacggtccaggtcttcac |
| | | ggactcagactatcagctcccgtacgtgctcgggtcggctcacgagggctgcctcccgccg |
| | | ttcccagcggacgttttcatgattcctcagtacgggtatctgacgcttaatgatggaagcc |
| | | aggccgtgggtcgttcgtccttttactgcctggaatatttcccgtcgcaaatgctaagaac |
| | | gggtaacaacttccagttcagctacgagtttgagaacgtacctttccatagcagctacgct |
| | | cacagccaaagcctggaccgactaatgaatccactcatcgaccaatacttgtactatctct |
| | | caaagactattaacggttctggacagaatcaacaaacgctaaaattcagtgtggccggacc |
| | | cagcaacatggctgtccagggaagaaactacatacctggacccagctaccgacaacaacgt |
| | | gtctcaaccactgtgactcaaaacaacaacagcgaatttgcttggcctggagcttcttctt |
| | | gggctctcaatggacgtaatagcttgatgaatcctggacctgctatggccagccacaaaga |
| | | aggagaggaccgtttctttcctttgtctggatctttaatttttggcaaacaaggaactgga |
| | | agagacaacgtggatgcggacaaagtcatgataaccaacgaagaagaaattaaaactacta |
| | | acccggtagcaacggagtcctatggacaagtggccacaaaccaccagagtgcacaggcttc |
| | | gactgggacgcttcggcttcaggcgcagaccggctgggttcaaaaccaaggaatacttccg |
| | | ggtatggtttggcaggacagagatgtgtacctgcaaggacccatttgggccaaaattcctc |
| | | acacggacggcaactttcacccttctccgctgatgggagggtttggaatgaagcacccgcc |
| | | tcctcagatcctcatcaaaaacacacctgtacctgcCgatcctccaacggccttcaacaag |
| | | gacaagctgaactctttcatcacccagtattctactggccaagtcagcgtggagatcgagt |
| | | gggagctgcagaaggaaaacagcaagcgGtggaacccggagatccagtacacttccaacta |
| | | ttacaagtctaataatgttgaatttgctgttaatactgaaggtgtatatagtgaaccccgc |
| | | cccattggcaccagatacctgactcgtaatctgtaa |
| TTD-010 | 3633 | atggctgccgatggttatcttccagattggctcgaggacaaccttagtgaaggaattcgcg |
| 7 mer peptide | | agtggtgggctttgaaacctggagcccctcaacccaaggcaaatcaacaacatcaagacaa |
| underlined | | cgctcgaggtcttgtgcttccgggttacaaataccttggacccggcaacggactcgacaag |
| | | ggggagccggtcaacgcagcagacgcggcggccctcgagcacgacaaggcctacgaccagc |
| | | agctcaaggccggagacaacccgtacctcaagtacaaccacgccgacgccgagttccagga |
| | | gcggctcaaagaagatacgtcttttgggggcaacctcgggcgagcagtcttccaggccaaa |
| | | aagaggcttcttgaacctcttggtctggttgaggaagcggctaagacggctcctggaaaga |
| | | agaggcctgtagagcagtctcctcaggaaccggactcctccgcgggtattggcaaatcggg |
| | | tgcacagcccgctaaaaagagactcaatttcggtcagactggcgacacagagtcagtccca |
| | | gaccctcaaccaatcggagaacctcccgcagccccctcaggtgtgggatctcttacaatgg |
| | | cttcaggtggtggcgcaccagtggcagacaataacgaaggtgccgatggagtgggtagttc |
| | | ctcgggaaattggcattgcgattcccaatggctgggggacagagtcatcaccaccagcacc |
| | | cgaacctgggccctgcccacctacaacaatcacctctacaagcaaatctccaacagcacat |
| | | ctggaggatcttcaaatgacaacgcctacttcggctacagcaccccctgggggtattttga |
| | | cttcaacagattccactgccacttctcaccacgtgactggcagcgactcatcaacaacaac |
| | | tggggattccggcctaagcgactcaacttcaagctcttcaacattcaggtcaaagaggtta |
| | | cggacaacaatggagtcaagaccatcgccaataaccttaccagcacggtccaggtcttcac |
| | | ggactcagactatcagctcccgtacgtgctcgggtcggctcacgagggctgcctcccgccg |
| | | ttcccagcggacgttttcatgattcctcagtacgggtatctgacgcttaatgatggaagcc |
| | | aggccgtgggtcgttcgtccttttactgcctggaatatttcccgtcgcaaatgctaagaac |
| | | gggtaacaacttccagttcagctacgagtttgagaacgtacctttccatagcagctacgct |
| | | cacagccaaagcctggaccgactaatgaatccactcatcgaccaatacttgtactatctct |
| | | caaagactattaacggttctggacagaatcaacaaacgctaaaattcagtgtggccggacc |
| | | cagcaacatggctgtccagggaagaaactacatacctggacccagctaccgacaacaacgt |
| | | gtctcaaccactgtgactcaaaacaacaacagcgaatttgcttggcctggagcttcttctt |
| | | gggctctcaatggacgtaatagcttgatgaatcctggacctgctatggccagccacaaaga |
| | | aggagaggaccgtttctttcctttgtctggatctttaatttttggcaaacaaggaactgga |
| | | agagacaacgtggatgcggacaaagtcatgataaccaacgaagaagaaattaaaactacta |
| | | acccggtagcaacggagtcctatggacaagtggccacaaaccaccagagtgcgcaggcgta |
| | | ttcgacggatgagaggatgcaggcgcagaccggctgggttcaaaaccaaggaatacttccg |
| | | ggtatggtttggcaggacagagatgtgtacctgcaaggacccatttgggccaaaattcctc |
| | | acacggacggcaactttcacccttctccgctgatgggagggtttggaatgaagcacccgcc |
| | | tcctcagatcctcatcaaaaacacacctgtacctgcCgatcctccaacggccttcaacaag |
| | | gacaagctgaactctttcatcacccagtattctactggccaagtcagcgtggagatcgagt |
| | | gggagctgcagaaggaaaacagcaagcgGtggaacccggagatccagtacacttccaacta |
| | | ttacaagtctaataatgttgaatttgctgttaatactgaaggtgtatatagtgaaccccgc |
| | | cccattggcaccagatacctgactcgtaatctgtaa |
| TTD-011 | 3634 | atggctgccgatggttatcttccagattggctcgaggacaaccttagtgaaggaattcgcg |
| 7 mer peptide | | agtggtgggctttgaaacctggagcccctcaacccaaggcaaatcaacaacatcaagacaa |
| underlined | | cgctcgaggtcttgtgcttccgggttacaaataccttggacccggcaacggactcgacaag |
| | | ggggagccggtcaacgcagcagacgcggcggccctcgagcacgacaaggcctacgaccagc |
| | | agctcaaggccggagacaacccgtacctcaagtacaaccacgccgacgccgagttccagga |
| | | gcggctcaaagaagatacgtcttttgggggcaacctcgggcgagcagtcttccaggccaaa |
| | | aagaggcttcttgaacctcttggtctggttgaggaagcggctaagacggctcctggaaaga |
| | | agaggcctgtagagcagtctcctcaggaaccggactcctccgcgggtattggcaaatcggg |
| | | tgcacagcccgctaaaaagagactcaatttcggtcagactggcgacacagagtcagtccca |
| | | gaccctcaaccaatcggagaacctcccgcagccccctcaggtgtgggatctcttacaatgg |
| | | cttcaggtggtggcgcaccagtggcagacaataacgaaggtgccgatggagtgggtagttc |
| | | ctcgggaaattggcattgcgattcccaatggctgggggacagagtcatcaccaccagcacc |
| | | cgaacctgggccctgcccacctacaacaatcacctctacaagcaaatctccaacagcacat |
| | | ctggaggatcttcaaatgacaacgcctacttcggctacagcaccccctgggggtattttga |
| | | cttcaacagattccactgccacttctcaccacgtgactggcagcgactcatcaacaacaac |
| | | tggggattccggcctaagcgactcaacttcaagctcttcaacattcaggtcaaagaggtta |
| | | cggacaacaatggagtcaagaccatcgccaataaccttaccagcacggtccaggtcttcac |
| | | ggactcagactatcagctcccgtacgtgctcgggtcggctcacgagggctgcctcccgccg |
| | | ttcccagcggacgttttcatgattcctcagtacgggtatctgacgcttaatgatggaagcc |
| | | aggccgtgggtcgttcgtccttttactgcctggaatatttcccgtcgcaaatgctaagaac |
| | | gggtaacaacttccagttcagctacgagtttgagaacgtacctttccatagcagctacgct |
| | | cacagccaaagcctggaccgactaatgaatccactcatcgaccaatacttgtactatctct |
| | | caaagactattaacggttctggacagaatcaacaaacgctaaaattcagtgtggccggacc |
| | | cagcaacatggctgtccagggaagaaactacatacctggacccagctaccgacaacaacgt |
| | | gtctcaaccactgtgactcaaaacaacaacagcgaatttgcttggcctggagcttcttctt |
| | | gggctctcaatggacgtaatagcttgatgaatcctggacctgctatggccagccacaaaga |
| | | aggagaggaccgtttctttcctttgtctggatctttaatttttggcaaacaaggaactgga |
| | | agagacaacgtggatgcggacaaagtcatgataaccaacgaagaagaaattaaaactacta |
| | | acccggtagcaacggagtcctatggacaagtggccacaaaccaccagagtgcgcaggcgta |
| | | ttcgacggatgagaggaagcaggcgcagaccggctgggttcaaaaccaaggaatacttccg |
| | | ggtatggtttggcaggacagagatgtgtacctgcaaggacccatttgggccaaaattcctc |
| | | acacggacggcaactttcacccttctccgctgatgggagggtttggaatgaagcacccgcc |
| | | tcctcagatcctcatcaaaaacacacctgtacctgcCgatcctccaacggccttcaacaag |
| | | gacaagctgaactctttcatcacccagtattctactggccaagtcagcgtggagatcgagt |
| | | gggagctgcagaaggaaaacagcaagcgGtggaacccggagatccagtacacttccaacta |
| | | ttacaagtctaataatgttgaatttgctgttaatactgaaggtgtatatagtgaaccccgc |
| | | cccattggcaccagatacctgactcgtaatctgtaa |
| TTD-012 | 3635 | atggctgccgatggttatcttccagattggctcgaggacaaccttagtgaaggaattcgcg |
| 7 mer peptide | | agtggtgggctttgaaacctggagcccctcaacccaaggcaaatcaacaacatcaagacaa |
| underlined | | cgctcgaggtcttgtgcttccgggttacaaataccttggacccggcaacggactcgacaag |
| | | ggggagccggtcaacgcagcagacgcggcggccctcgagcacgacaaggcctacgaccagc |
| | | agctcaaggccggagacaacccgtacctcaagtacaaccacgccgacgccgagttccagga |
| | | gcggctcaaagaagatacgtcttttgggggcaacctcgggcgagcagtcttccaggccaaa |
| | | aagaggcttcttgaacctcttggtctggttgaggaagcggctaagacggctcctggaaaga |
| | | agaggcctgtagagcagtctcctcaggaaccggactcctccgcgggtattggcaaatcggg |
| | | tgcacagcccgctaaaaagagactcaatttcggtcagactggcgacacagagtcagtccca |
| | | gaccctcaaccaatcggagaacctcccgcagccccctcaggtgtgggatctcttacaatgg |
| | | cttcaggtggtggcgcaccagtggcagacaataacgaaggtgccgatggagtgggtagttc |
| | | ctcgggaaattggcattgcgattcccaatggctgggggacagagtcatcaccaccagcacc |
| | | cgaacctgggccctgcccacctacaacaatcacctctacaagcaaatctccaacagcacat |
| | | ctggaggatcttcaaatgacaacgcctacttcggctacagcaccccctgggggtattttga |
| | | cttcaacagattccactgccacttctcaccacgtgactggcagcgactcatcaacaacaac |
| | | tggggattccggcctaagcgactcaacttcaagctcttcaacattcaggtcaaagaggtta |
| | | cggacaacaatggagtcaagaccatcgccaataaccttaccagcacggtccaggtcttcac |
| | | ggactcagactatcagctcccgtacgtgctcgggtcggctcacgagggctgcctcccgccg |
| | | ttcccagcggacgttttcatgattcctcagtacgggtatctgacgcttaatgatggaagcc |
| | | aggccgtgggtcgttcgtccttttactgcctggaatatttcccgtcgcaaatgctaagaac |
| | | gggtaacaacttccagttcagctacgagtttgagaacgtacctttccatagcagctacgct |
| | | cacagccaaagcctggaccgactaatgaatccactcatcgaccaatacttgtactatctct |
| | | caaagactattaacggttctggacagaatcaacaaacgctaaaattcagtgtggccggacc |
| | | cagcaacatggctgtccagggaagaaactacatacctggacccagctaccgacaacaacgt |
| | | gtctcaaccactgtgactcaaaacaacaacagcgaatttgcttggcctggagcttcttctt |
| | | gggctctcaatggacgtaatagcttgatgaatcctggacctgctatggccagccacaaaga |
| | | aggagaggaccgtttctttcctttgtctggatctttaatttttggcaaacaaggaactgga |
| | | agagacaacgtggatgcggacaaagtcatgataaccaacgaagaagaaattaaaactacta |
| | | acccggtagcaacggagtcctatggacaagtggccacaaaccaccagagtgcacaggctta |
| | | tgtttcgtctgttaagatgcaggcgcagaccggctgggttcaaaaccaaggaatacttccg |
| | | ggtatggtttggcaggacagagatgtgtacctgcaaggacccatttgggccaaaattcctc |
| | | acacggacggcaactttcacccttctccgctgatgggagggtttggaatgaagcacccgcc |
| | | tcctcagatcctcatcaaaaacacacctgtacctgcCgatcctccaacggccttcaacaag |
| | | gacaagctgaactctttcatcacccagtattctactggccaagtcagcgtggagatcgagt |
| | | gggagctgcagaaggaaaacagcaagcgGtggaacccggagatccagtacacttccaacta |
| | | ttacaagtctaataatgttgaatttgctgttaatactgaaggtgtatatagtgaaccccgc |
| | | cccattggcaccagatacctgactcgtaatctgtaa |

In some embodiments, an AAV capsid variant described herein comprises the amino acid sequence of any one of SEQ ID NOs: 3636-3647, or an amino acid sequence with at least 80% (e.g., at least about 85, 90, 95, 96, 97, 98, or 99%) sequence identity thereto. In some embodiments, an AAV capsid variant described herein comprises the amino acid sequence of SEQ ID NO: 3636, or an amino acid sequence with at least 80% (e.g., at least about 85, 90, 95, 96, 97, 98, or 99%) sequence identity thereto. In some embodiments, an AAV capsid variant described herein comprises the amino acid sequence of SEQ ID NO: 3639, or an amino acid sequence with at least 80% (e.g., at least about 85, 90, 95, 96, 97, 98, or 99%) sequence identity thereto.

In some embodiments, the polynucleotide encoding an AAV capsid variant described herein comprises the nucleotide sequence of any one of SEQ ID NOs: 3623-3635, or a nucleotide sequence with at least 80% (e.g., at least about 85, 90, 95, 96, 97, 98, or 990%) sequence identity thereto. In some embodiments, the polynucleotide encoding an AAV capsid variant described herein comprises the nucleotide sequence of SEQ ID NO: 3623, or a nucleotide sequence with at least 80% (e.g., at least about 85, 90, 95, 96, 97, 98, or 99%) sequence identity thereto. In some embodiments, the polynucleotide encoding an AAV capsid variant described herein comprises the nucleotide sequence of SEQ ID NO: 3627, or a nucleotide sequence with at least 80% (e.g., at least about 85, 90, 95, 96, 97, 98, or 99%) sequence identity thereto. In some embodiments, the nucleic acid sequence encoding an AAV capsid variant described herein is codon optimized.

In some embodiments, the AAV capsid variant comprises a VP2 protein comprising the amino acid sequence corresponding to positions 138-743, of any one of SEQ ID NOs: 3636-3647, or a sequence with at least 80% (e.g., at least about 85, 90, 95, 96, 97, 98, or 99%) sequence identity thereto. In some embodiments, the AAV capsid comprises a VP3 protein comprising the amino acid sequence corresponding to positions 203-743, of any one of SEQ ID NOs: 3636-3647, or a sequence with at least 80% (e.g., at least about 85, 90, 95, 96, 97, 98, or 99%) sequence identity thereto.

In some embodiments, an AAV capsid variant, described herein has an increased tropism for a CNS cell or tissue, e.g., a brain cell, brain tissue, spinal cord cell, or spinal cord tissue, relative to the tropism of a reference sequence comprising the amino acid sequence of SEQ ID NO: 138.

In some embodiments, an AAV capsid variant, described herein transduces a brain region, e.g., selected from dentate nucleus, cerebellar cortex, cerebral cortex, brain stem, hippocampus, thalamus and putamen. In some embodiments, the level of transduction of said brain region is at least 5, 10, 50, 100, 200, 500, 1,000, 2,000, 5,000, or 10,000-fold greater as compared to a reference sequence of SEQ ID NO: 138.

In some embodiments, an AAV capsid variant described herein is enriched at least about 5, 6, 7, 8, 9, or 10-fold, in the brain compared to a reference sequence of SEQ ID NO: 138. In some embodiments, an AAV capsid variant described herein is enriched at least about 20, 30, 40, or 50-fold in the brain compared to a reference sequence of SEQ ID NO: 138. In some embodiments, an AAV capsid variant described herein is enriched at least about 100, 200, 300, or 400-fold in the brain compared to a reference sequence of SEQ ID NO: 138.

In some embodiments, an AAV capsid variant described herein delivers an increased level of viral genomes to a brain region. In some embodiments, the level of viral genomes is increased by at least 5, 10, 20, 30, 40 or 50-fold, as compared to a reference sequence of SEQ ID NO: 138. In some embodiments, the brain region comprises a frontal cortex, sensory cortex, motor cortex, putamen, thalamus, cerebellar cortex, dentate nucleus, caudate, and/or hippocampus.

In some embodiments, an AAV capsid variant described herein delivers an increased level of a payload to a brain region. In some embodiments, the level of the payload is increased by at least 5, 10, 50, 100, 200, 500, 1,000, 2,000, 5,000, or 10,000-fold, as compared to a reference sequence of SEQ ID NO: 138. In some embodiments, the brain region comprises a frontal cortex, sensory cortex, motor cortex, putamen, thalamus, cerebellar cortex, dentate nucleus, caudate, and/or hippocampus.

In some embodiments, an AAV capsid variant described herein delivers an increased level of a payload to a spinal cord region. In some embodiments, the level of the payload is increased by at least 10, 20, 50, 100, 200, 300, 400, 500, 600, 700, 800 or 900-fold, as compared to a reference sequence of SEQ ID NO: 138. In some embodiments, the spinal cord region comprises a cervical, thoracic, and/or lumbar region.

In some embodiments, an AAV capsid variant described herein shows preferential transduction in a brain region relative to the transduction in the dorsal root ganglia (DRG).

In some embodiments, an AAV capsid variant described herein has an increased tropism for a muscle cell or tissue, e.g., a heart cell or tissue, relative to the tropism of a reference sequence comprising the amino acid sequence of SEQ ID NO: 138. In some embodiments, the AAV capsid variant delivers an increased level of a payload to a muscle region. In some embodiments, the payload is increased by at least 10, 15, 20, 30, or 40-fold, as compared to a reference sequence of SEQ ID NO: 138. In some embodiments, the muscle region comprises a heart muscle, quadriceps muscle, and/or a diaphragm muscle region. In some embodiments, the muscle region comprises a heart muscle region, e.g., a heart atrium muscle region or a heart ventricle muscle region.

In some embodiments, an AAV capsid variant of the present disclosure is isolated, e.g., recombinant. In some embodiments, a polynucleotide encoding an AAV capsid variant of the present disclosure is isolated, e.g., recombinant.

Also provided herein are polynucleotide sequences encoding any of the AAV capsid variants described above and AAV particles, vectors, and cells comprising the same.

### Viral Genome Component: Inverted Terminal Repeats (ITRs)

In some embodiments, the viral genome may comprise at least one inverted terminal repeat (ITR) region. In some embodiments, the viral genome comprises at least one ITR region and a nucleic acid encoding a payload, e.g., an antibody (e.g., an anti-tau antibody). In some embodiments, viral genome comprises two ITRs. In some embodiments, the two ITRs flank the nucleic acid encoding the transgene at the 5′ and 3′ ends. In some embodiments, the ITR functions as an origin of replication comprising recognition sites for replication. In some embodiments, the ITRs comprise sequence regions which can be complementary and symmetrically arranged. In some embodiments, the ITR incorporated into viral genome may be comprised of naturally occurring nucleic acid sequences or recombinantly derived nucleic acid sequences.

In some embodiments, the ITR may be of the same AAV serotype as the capsid, e.g., a capsid protein selected from any of the AAV serotypes listed in Table 2, or a functional variant thereof. In some embodiments, the ITR may be of a different AAV serotype than the capsid protein. In some embodiments, the AAV particle comprises a viral genome comprising two ITRs wherein the two ITRs of viral genome are of the same AAV serotype. In other embodiments, the two ITRs of a viral genome are of different AAV serotypes. In some embodiments both ITRs of the viral genome of the AAV particle are AAV2 ITRs or a functional variant thereof.

In some embodiments, the ITR comprises about 120-140 nucleotides in length, e.g., about 130 nucleotides in length. In some embodiments, the ITR comprises about 140-150 nucleotides in length, about 141 nucleotides in length. In some embodiments, the viral genome comprises an ITR region comprising the nucleotide sequence of any of the sequences provided in Table 10 or a nucleotide sequence with at least 70%, 75%, 80%, 85%, 90%, 95% or 99% sequence identity thereto. In some embodiments, the viral genome comprises two ITR regions comprising the nucleotide sequence of any of the sequences provided in Table 10 or a nucleotide sequence with at least 70%, 75%, 80%, 85%, 90%, 95% or 99% sequence identity thereto, wherein the first and second ITR comprise the same sequence or wherein the first and second ITR comprise different sequences.

In some embodiments, the AAV particle viral genome comprises a 5′ inverted terminal repeat (5′ ITR) sequence region. In some embodiments, the viral genome comprises a 3′ inverted terminal repeat (3′ ITR) sequence region. Non-limiting examples of 5′ ITR and 3′ ITR sequence regions are described in Table 10.

TABLE 10
Inverted Terminal Repeat (ITR) Sequence Regions

| SEQ | |
|---|---|
| ID NO | Sequence |
| 1035 | ctgcgcgctcgctcgctcactgaggccgcccgggcaaagcccgggcgtcgggcgacctttggtcg |
| | cccggcctcagtgagcgagcgagcgcgcagagagggagtggccaactccatcactaggggttcct |
| 1036 | cctgcaggcagctgcgcgctcgctcgctcactgaggccgcccgggcaaagcccgggcgtcgggcg |
| | acctttggtcgcccggcctcagtgagcgagcgagcgcgcagagagggagtggccaactccatcac |
| | taggggttcct |
| 1037 | aggaacccctagtgatggagttggccactccctctctgcgcgctcgctcgctcactgaggccggg |
| | cgaccaaaggtcgcccgacgcccgggctttgcccgggcggcctcagtgagcgagcgagcgcgcag |
| 1038 | aggaacccctagtgatggagttggccactccctctctgcgcgctcgctcgctcactgaggccggg |
| | cgaccaaaggtcgcccgacgcccgggctttgcccgggcggcctcagtgagcgagcgagcgcgcag |
| | ctgcctgcagg |

In some embodiments, the ITR comprises the nucleotide sequence of any one of SEQ ID NOs: 1035-1038, or a sequence with at least 70%, 75%, 80%, 85%, 90%, 95% or 99% sequence identity thereto. In some embodiments, the ITR comprises the nucleotide sequence of SEQ ID NO: 1035 or a nucleotide sequence with at least 70%, 75%, 80%, 85%, 90%, 95% or 99% sequence identity thereto. In some embodiments, the ITR comprises the nucleotide sequence of SEQ ID NO: 1036 or a nucleotide sequence with at least 70%, 75%, 80%, 85%, 90%, 95% or 99% sequence identity thereto. In some embodiments, the ITR comprises the nucleotide sequence of SEQ ID NO: 1037 or a nucleotide sequence with at least 70%, 75%, 80%, 85%, 90%, 95% or 99% sequence identity thereto. In some embodiments, the ITR comprises the nucleotide sequence of SEQ ID NO: 1038 or a nucleotide sequence with at least 70/6, 75%, 80%, 85%, 90%, 95% or 99% sequence identity thereto.

In some embodiments, the viral genome comprises an ITR, e.g., a 5′ ITR, comprising the nucleotide sequence of SEQ ID NO: 1035 or a sequence with at least with at least 70%, 75%, 80%, 85%, 90%, 95% or 99% sequence identity thereto; and/or an ITR, e.g., a 3′ ITR, comprising the nucleotide sequence of SEQ ID NO: 1037 or a nucleotide sequence at least 70/6, 75%, 80%, 85%, 90%, 95% or 99% sequence identity thereto.

### Viral Genome Component: Promoters

In some embodiments, the viral genome may comprise an element to enhance the transgene target specificity and/or expression (See e.g., Powell et al. Viral Expression Cassette Elements to Enhance Transgene Target Specificity and Expression in Gene Therapy, 2015; the contents of which are herein incorporated by reference in its entirety). In some embodiments, the AAV particle viral genome may comprise an element to enhance the transgene target specificity and/or expression comprise a promoter, an enhancer, e.g., a CMV enhancer, or both. In some embodiments, the AAV particle viral genome comprises a promoter operably linked to a transgene encoded by a nucleic acid molecule encoding a payload, e.g., antibody (e.g., an anti-tau antibody). In some embodiments, the AAV particle viral genome comprises an enhancer, e.g., a CMV enhancer. In some embodiment, the AAV particle viral genome comprises at least two promoters.

In some embodiments, the viral genome comprises a ubiquitous promoter. In some embodiments, the viral genome comprises a tissue specific promoter.

In some embodiments, the viral genome comprises a promoter that is species specific, inducible, tissue-specific, and/or cell cycle-specific (e.g., as described in Parr et al., Nat. Med. 3:1145-9 (1997); the contents of which are herein incorporated by reference in their entirety). In some embodiments, the viral genome comprises a promoter that is sufficient for expression, e.g., in a target cell, of a payload (e.g., an antibody, e.g., an anti-tau antibody) encoded by a transgene.

In some embodiments, the promoter may be a naturally occurring promoter, or a non-naturally occurring promoter. In some embodiments, the promoter is from a naturally expressed protein. In some embodiments, the promoter is an engineered promoter. In some embodiments, the promoter comprises a viral promoter, plant promoter, and/or a mammalian promoter. In some embodiments, the promoter may be a human promoter. In some embodiments, the promoter may be truncated. In some embodiments, the promoter is not a cell specific promoter.

In some embodiments, the promoter results in expression in one or more, e.g., multiple, cells and/or tissues, e.g., a ubiquitous promoter. In some embodiments, a promoter that results in expression in one or more tissues includes but is not limited to a human elongation factor 1α-subunit (EF1α) promoter, a cytomegalovirus (CMV) immediate-early enhancer and/or promoter, a chicken β-actin (CBA) promoter and its derivative CAG, a β glucuronidase (GUSB) promoter, or ubiquitin C (UBC) promoter. In some embodiments, a tissue-specific expression elements can be used to restrict expression to certain cell types such as, but not limited to, muscle specific promoters, B cell promoters, monocyte promoters, leukocyte promoters, macrophage promoters, pancreatic acinar cell promoters, endothelial cell promoters, lung tissue promoters, astrocyte promoters, or nervous system promoters which can be used to restrict expression to neurons, astrocytes, or oligodendrocytes. In some embodiments, the promoter is a ubiquitous promoter as described in Yu et al. (Molecular Pain 2011, 7:63), Soderblom et al. (E. Neuro 2015), Gill et al., (Gene Therapy 2001, Vol. 8, 1539-1546), and Husain et al. (Gene Therapy 2009), each of which are incorporated by reference in their entirety. In some embodiments, the promoter is a ubiquitous promoter chosen from CMV, CBA (including derivatives CAG, CB6, CBh, etc.), EF-1α, PGK, UBC, GUSB (hGBp), or UCOE (promoter of HNRPA2B1-CBX3).

In some embodiments, the promoter is a muscle-specific promoter, e.g., a promoter that results in expression in a muscle cell. In some embodiments, a muscle-specific promoter includes but is not limited to a mammalian muscle creatine kinase (MCK) promoter, a mammalian desmin (DES) promoter, a mammalian troponin I (TNNI2) promoter, a synthetic C5-12 promoter, and a mammalian skeletal alpha-actin (ASKA) promoter (see, e.g. U.S. Patent Publication US20110212529, the contents of which are herein incorporated by reference in their entirety).

In some embodiments, the promoter is a nervous system specific promoter, e.g., a promoter that results in expression of a payload in a neuron, an astrocyte, and/or an oligodendrocyte. In some embodiments, a nervous system specific promoter that results in expression in neurons includes but is not limited to a neuron-specific enolase (NSE) promoter, a platelet-derived growth factor (PDGF) promoter, a platelet-derived growth factor B-chain (PDGF-β) promoter, a synapsin (Syn) promoter, a methyl-CpG binding protein 2 (MeCP2) promoter, a Ca²⁺/calmodulin-dependent protein kinase II (CaMKII) promoter, a metabotropic glutamate receptor 2 (mGluR2) promoter, a neurofilament light (NFL) or heavy (NFH) promoter, a β-globin minigene nβ2 promoter, a preproenkephalin (PPE) promoter, a enkephalin (Enk) promoter, and an excitatory amino acid transporter 2 (EAAT2) promoter. In some embodiments, a nervous system specific promoter that results in expression in astrocytes includes but is not limited to a glial fibrillary acidic protein (GFAP) promoter and a EAAT2 promoter. In some embodiments, a nervous system specific promoter that results in expression in oligodendrocytes includes but is not limited to a myelin basic protein (MBP) promoter. In some embodiments, the viral genome comprises a nervous system specific promoter as described in Husain et al. (Gene Therapy 2009), Passini and Wolfe (J. Virol. 2001, 12382-12392), Xu et al. (Gene Therapy 2001, 8, 1323-1332), Drews et al. (Mamm Genome (2007) 18:723-731), and Raymond et al. (Journal of Biological Chemistry (2004) 279(44) 46234-46241), each of which are incorporated by reference in their entirety.

In some embodiments, the promoter is a liver promoter, e.g. a promoter that results in expression a liver cell. In some embodiments, the liver promoter is chosen from human α-1-antitrypsin (hAAT) or thyroxine binding globulin (TBG). In some embodiments, the viral genome comprises an RNA pol III promoter. In some embodiments, the RNA pol III promoter is chosen from U6 or H1.

In some embodiments, the viral genome comprises two promoters. As a non-limiting example, the promoters are an EF1α promoter and a CMV promoter.

In some embodiments, the promoter is a ubiquitin c (UBC) promoter. The UBC promoter may have a size of 300-350 nucleotides. As a non-limiting example, the UBC promoter is 332 nucleotides. In some embodiments, the promoter is a β-glucuronidase (GUSB) promoter. The GUSB promoter may have a size of 350-400 nucleotides. As a non-limiting example, the GUSB promoter is 378 nucleotides. In some embodiments, the promoter is a neurofilament light (NFL) promoter. The NFL promoter may have a size of 600-700 nucleotides. As a non-limiting example, the NFL promoter is 650 nucleotides. In some embodiments, the promoter is a neurofilament heavy (NFH) promoter. The NFH promoter may have a size of 900-950 nucleotides. As a non-limiting example, the NFH promoter is 920 nucleotides. In some embodiments, the promoter is a scn8a promoter. The scn8a promoter may have a size of 450-500 nucleotides. As a non-limiting example, the scn8a promoter is 470 nucleotides. In some embodiments, the promoter is a phosphoglycerate kinase 1 (PGK) promoter.

In some embodiments, the viral genome comprises a promoter chosen from a CAG promoter, a CBA promoter (e.g., a minimal CBA promoter), a CB promoter, a CMV(IE) promoter and/or enhancer, a GFAP promoter, a synapsin promoter, an ICAM2 promoter, or a functional variant thereof. In some embodiments, the viral genome comprises a CAG promoter, a CMVie enhancer, and a minimal CBA promoter. In some embodiments, the viral genome comprises a CMV(IE) promoter and a CB promoter.

In some embodiments, the viral genome comprises an enhancer element, a promoter and/or a 5′UTR intron. The enhancer element, also referred to herein as an “enhancer,” may be, but is not limited to, a CMV enhancer, the promoter may be, but is not limited to, a CMV, CBA, UBC, GUSB, NSE, Synapsin, MeCP2, and GFAP promoter and the 5′UTR/intron may be, but is not limited to, SV40, and CBA-MVM. As a non-limiting example, the enhancer, promoter and/or intron used in combination may be: (1) CMV enhancer, CMV promoter, SV40 5′UTR intron; (2) CMV enhancer, CBA promoter, SV 40 5′UTR intron; (3) CMV enhancer, CBA promoter, CBA-MVM 5′UTR intron; (4) UBC promoter; (5) GUSB promoter; (6) NSE promoter; (7) Synapsin promoter; (8) MeCP2 promoter; and (9) GFAP promoter.

In some embodiments, a viral genome encoding an antibody that binds to tau described herein comprises a CBA promoter or a functional variant thereof. In some embodiments, the promoter comprises the nucleotide sequence of SEQ ID NO: 1042, or a nucleotide sequence at least 70%, 75%, 80%, 85%, 90%, 95%, 96%, 97%, 98%, or 99% identical thereto. In some embodiments, the promoter comprises the nucleotide sequence of SEQ ID NO: 1042, or a nucleotide sequence at least 95% identical thereto.

In some embodiments, a viral genome encoding an antibody that binds to tau described herein comprises a CMVie enhancer or a functional variant thereof. In some embodiments, the enhancer comprises the nucleotide sequence of SEQ ID NO: 1050, or a nucleotide sequence at least 70%, 75%, 80%, 85%, 90%, 95%, 96%, 97%, 98%, or 99% identical thereto. In some embodiments, the promoter comprises the nucleotide sequence of SEQ ID NO: 1050, or a nucleotide sequence at least 95% identical thereto.

In some embodiments, a viral genome encoding an antibody that binds to tau described herein comprises a CMVie enhancer or a functional variant thereof and a CBA promoter or a functional variant thereof. In some embodiments, the viral genome comprises (i) an enhancer which comprises the nucleotide sequence of SEQ ID NO: 1050, or a nucleotide sequence at least 70%, 75%, 80%, 85%, 90%, 95%, 96%, 97%, 98%, or 99% identical thereto; and (ii) a promoter which comprises the nucleotide sequence of SEQ ID NO: 1042, or a nucleotide sequence at least 70%, 75%, 80%, 85%, 90, 95%, 96%, 97%, 98%, or 99% identical thereto.

TABLE 11
Exemplary Promoter Sequences

| | SEQ | |
|---|---|---|
| | ID | |
| Name | NO | Sequence |
| CAG | 1039 | CTAGTTATTAATAGTAATCAATTACGGGGTCATTAGTTCATAGCCCATATATGGAGTTCCGCG |
| Promoter | | TTACATAACTTACGGTAAATGGCCCGCCTGGCTGACCGCCCAACGACCCCCGCCCATTGACGT |
| Variant 1 | | CAATAATGACGTATGTTCCCATAGTAACGCCAATAGGGACTTTCCATTGACGTCAATGGGTGG |
| | | AGTATTTACGGTAAACTGCCCACTTGGCAGTACATCAAGTGTATCATATGCCAAGTACGCCCC |
| | | CTATTGACGTCAATGACGGTAAATGGCCCGCCTGGCATTATGCCCAGTACATGACCTTATGGG |
| | | ACTTTCCTACTTGGCAGTACATCTACGTATTAGTCATCGCTATTACCATGGTCGAGGTGAGCC |
| | | CCACGTTCTGCTTCACTCTCCCCATCTCCCCCCCCTCCCCACCCCCAATTTTGTATTTATTTA |
| | | TTTTTTAATTATTTTGTGCAGCGATGGGGGCGGGGGGGGGGGGGGGGCGCGCGCCAGGCGGGG |
| | | CGGGGCGGGGCGAGGGGCGGGGCGGGGCGAGGCGGAGAGGTGCGGCGGCAGCCAATCAGAGCG |
| | | GCGCGCTCCGAAAGTTTCCTTTTATGGCGAGGCGGCGGCGGCGGCGGCCCTATAAAAAGCGAA |
| | | GCGCGCGGCGGGCGGGAGTCGCTGCGCGCTGCCTTCGCCCCGTGCCCCGCTCCGCCGCCGCCT |
| | | CGCGCCGCCCGCCCCGGCTCTGACTGACCGCGTTACTCCCACAGGTGAGCGGGCGGGACGGCC |
| | | CTTCTCCTCCGGGCTGTAATTAGCGCTTGGTTTAATGACGGCTTGTTTCTTTTCTGTGGCTGC |
| | | GTGAAAGCCTTGAGGGGCTCCGGGAGGGCCCTTTGTGCGGGGGGAGCGGCTCGGGGGGTGCGT |
| | | GCGTGTGTGTGTGCGTGGGGAGCGCCGCGTGCGGCTCCGCGCTGCCCGGCGGCTGTGAGCGCT |
| | | GCGGGCGCGGCGCGGGGCTTTGTGCGCTCCGCAGTGTGCGCGAGGGGAGCGCGGCCGGGGGCG |
| | | GTGCCCCGCGGTGCGGGGGGGGCTGCGAGGGGAACAAAGGCTGCGTGCGGGGTGTGTGCGTGG |
| | | GGGGGTGAGCAGGGGGTGTGGGCGCGTCGGTCGGGCTGCAACCCCCCCTGCACCCCCCTCCCC |
| | | GAGTTGCTGAGCACGGCCCGGCTTCGGGTGCGGGGCTCCGTACGGGGCGTGGCGCGGGGCTCG |
| | | CCGTGCCGGGCGGGGGGTGGCGGCAGGTGGGGGTGCCGGGCGGGGCGGGGCCGCCTCGGGCCG |
| | | GGGAGGGCTCGGGGGAGGGGCGCGGCGGCCCCCGGAGCGCCGGCGGCTGTCGAGGCGCGGCGA |
| | | GCCGCAGCCATTGCCTTTTATGGTAATCGTGCGAGAGGGCGCAGGGACTTCCTTTGTCCCAAA |
| | | TCTGTGCGGAGCCGAAATCTGGGAGGCGCCGCCGCACCCCCTCTAGCGGGCGCGGGGCGAAGC |
| | | GGTGCGGCGCCGGCAGGAAGGAAATGGGCGGGGAGGGCCTTCGTGCGTCGCCGCGCCGCCGTC |
| | | CCCTTCTCCCTCTCCAGCCTCGGGGCTGTCCGCGGGGGGACGGCTGCCTTCGGGGGGGACGGG |
| | | GCAGGGCGGGGTTCGGCTTCTGGCGTGTGACCGGCGGCTCTAGAGCCTCTGCTAACCATGTTC |
| | | ATGCCTTCTTCTTTTTCCTACAGCTCCTGGGCAACGTGCTGGTTATTGTGCTGTCTCATCATT |
| | | TTGGCAAAGAATTC |
| CMVie | 1040 | TAGCCCATATATGGAGTTCCGCGTTACATAACTTACGGTAAATGGCCCGCCTGGCTGACCGCC |
| Promoter | | CAACGACCCCCGCCCATTGACGTCAATAATGACGTATGTTCCCATAGTAACGCCAATAGGGAC |
| Variant | | TTTCCATTGACGTCAATGGGTGGAGTATTTACGGTAAACTGCCCACTTGGCAGTACATCAAGT |
| | | GTATCATATGCCAAGTACGCCCCCTATTGACGTCAATGACGGTAAATGGCCCGCCTGGCATTA |
| | | TGCCCAGTACATGACCTTATGGGACTTTCCTACTTGGCAGTACATCT |
| CBA | 1041 | CATGGTCGAGGTGAGCCCCACGTTCTGCTTCACTCTCCCCATCTCCCCCCCCTCCCCACCCCC |
| Promoter | | AATTTTGTATTTATTTATTTTTTAATTATTTTGTGCAGCGATGGGGGCGGGGGGGGGGGGGGG |
| Variant | | GCGCGCGCCAGGCGGGCGGGGCGGGGGGCGAGGGGCGGGGGGGGCGAGGCGGAGAGGTGCGGC |
| | | GGCAGCCAATCAGAGCGGCGCGCTCCGAAAGTTTCCTTTTATGGCGAGGCGGCGGCGGCGGCG |
| | | GCCCTATAAAAAGCGAAGCGCGCGGCGGGCG |
| CBA | 1042 | CCACGTTCTGCTTCACTCTCCCCATCTCCCCCCCCTCCCCACCCCCAATTTTGTATTTATTTA |
| Promoter | | TTTTTTAATTATTTTGTGCAGCGATGGGGGCGGGGGGGGGGGGCGCGCGCCAGGCGGGGCGGG |
| Variant 2 | | GCGGGGCGAGGGGCGGGGCGGGGCGAGGCGGAGAGGTGCGGCGGCAGCCAATCAGAGCGGCGC |
| | | GCTCCGAAAGTTTCCTTTTATGGCGAGGCGGCGGCGGCGGCGGCCCTATAAAAAGCGAAGCGC |
| | | GCGGCGGG |
| CBA | 1043 | CGACATTGATTATTGACTAGTTATTAATAGTAATCAATTACGGGGTCATTAGTTCATAGCCCA |
| Promoter | | TATATGGAGTTCCGCGTTACATAACTTACGGTAAATGGCCCGCCTGGCTGACCGCCCAACGAC |
| | | CCCCGCCCATTGACGTCAATAATGACGTATGTTCCCATAGTAACGCCAATAGGGACTTTCCAT |
| | | TGACGTCAATGGGTGGAGTATTTACGGTAAACTGCCCACTTGGCAGTACATCAAGTGTATCAT |
| | | ATGCCAAGTACGCCCCCTATTGACGTCAATGACGGTAAATGGCCCGCCTGGCATTATGCCCAG |
| | | TACATGACCTTATGGGACTTTCCTACTTGGCAGTACATCTACGTATTAGTCATCGCTATTACC |
| | | ATGTCGAGGCCACGTTCTGCTTCACTCTCCCCATCTCCCCCCCCTCCCCACCCCCAATTTTGT |
| | | ATTTATTTATTTTTTAATTATTTTGTGCAGCGATGGGGGCGGGGGGGGGGGGCGCGCGCCAGG |
| | | CGGGGCGGGGCGGGGCGAGGGGCGGGGCGGGGCGAGGCGGAGAGGTGCGGCGGCAGCCAATCA |
| | | GAGCGGCGCGCTCCGAAAGTTTCCTTTTATGGCGAGGCGGCGGCGGCGGCGGCCCTATAAAAA |
| | | GCGAAGCGCGCGGCGGGCGGGAGC |
| GFAP | 1044 | GATCTAACATATCCTGGTGTGGAGTAGCGGACGCTGCTATGACAGAGGCTCGGGGGCCTGAGC |
| Promoter | | TGGCTCTGTGAGCTGGGGAGGAGGCAGACAGCCAGGCCTTGTCTGCAAGCAGACCTGGCAGCA |
| | | TTGGGCTGGCCGCCCCCCAGGGCCTCCTCTTCATGCCCAGTGAATGACTCACCTTGGCACAGA |
| | | CACAATGTTCGGGGTGGGCACAGTGCCTGCTTCCCGCCGCACCCCAGCCCCCCTCAAATGCCT |
| | | TCCGAGAAGCCCATTGAGCAGGGGGCTTGCATTGCACCCCAGCCTGACAGCCTGGCATCTTGG |
| | | GATAAAAGCAGCACAGCCCCCTAGGGGCTGCCCTTGCTGTGTGGCGCCACCGGCGGTGGAGAA |
| | | CAAGGCTCTATTCAGCCTGTGCCCAGGAAAGGGGATCAGGGGATGCCCAGGCATGGACAGTGG |
| | | GTGGCAGGGGGGGAGAGGAGGGCTGTCTGCTTCCCAGAAGTCCAAGGACACAAATGGGTGAGG |
| | | GGAGAGCTCTCCCCATAGCTGGGCTGCGGCCCAACCCCACCCCCTCAGGCTATGCCAGGGGGT |
| | | GTTGCCAGGGGCACCCGGGCATCGCCAGTCTAGCCCACTCCTTCATAAAGCCCTCGCATCCCA |
| | | GGAGCGAGCAGAGCCAGAGCAGGTTGGAGAGGAGACGCATCACCTCCGCTGCTCGCGGGGATC |
| | | CTCTAG |
| Synuclein | 1045 | TAGTATCTGCAGAGGGCCCTGCGTATGAGTGCAAGTGGGTTTTAGGACCAGGATGAGGCGGGG |
| Promoter | | TGGGGGTGCCTACCTGACGACCGACCCCGACCCACTGGACAAGCACCCAACCCCCATTCCCCA |
| | | AATTGCGCATCCCCTATCAGAGAGGGGGAGGGGAAACAGGATGCGGCGAGGCGCGTGCGCACT |
| | | GCCAGCTTCAGCACCGCGGACAGTGCCTTCGCCCCCGCCTGGCGGCGCGCGCCACCGCCGCCT |
| | | CAGCACTGAAGGCGCGCTGACGTCACTCGCCGGTCCCCCGCAAACTCCCCTTCCCGGCCACCT |
| | | TGGTCGCGTCCGCGCCGCCGCCGGCCCAGCCGGACCGCACCACGCGAGGCGCGAGATAGGGGG |
| | | GCACGGGCGCGACCATCTGCGCTGCGGCGCCGGCGACTCAGCGCTGCCTCAGTCTGCGGTGGG |
| | | CAGCGGAGGAGTCGTGTCGTGCCTGAGAGCGCAGCTGTGCTCCTGGGCACCGCGCAGTCCGCC |
| | | CCCGCGGCTCCTGGCCAGACCACCCCTAGGACCCCCTGCCCCAAGTCGCAGCC |
| CMVie | 1046 | CTAGTCGACATTGATTATTGACTAGTTATTAATAGTAATCAATTACGGGGTCATTAGTTCATA |
| Promoter | | GCCCATATATGGAGTTCCGCGTTACATAACTTACGGTAAATGGCCCGCCTGGCTGACCGCCCA |
| Variant 2 | | ACGACCCCCGCCCATTGACGTCAATAATGACGTATGTTCCCATAGTAACGCCAATAGGGACTT |
| | | TCCATTGACGTCAATGGGTGGAGTATTTACGGTAAACTGCCCACTTGGCAGTACATCAAGTGT |
| | | ATCATATGCCAAGTACGCCCCCTATTGACGTCAATGACGGTAAATGGCCCGCCTGGCATTATG |
| | | CCCAGTACATGACCTTATGGGACTTTCCTACTTGGCAGTACATCTACGTATTAGTCATCGCTA |
| | | TTAC |
| CAG | 1047 | CTAGTCGACATTGATTATTGACTAGTTATTAATAGTAATCAATTACGGGGTCATTAGTTCATA |
| Promoter | | GCCCATATATGGAGTTCCGCGTTACATAACTTACGGTAAATGGCCCGCCTGGCTGACCGCCCA |
| Variant 2 | | ACGACCCCCGCCCATTGACGTCAATAATGACGTATGTTCCCATAGTAACGCCAATAGGGACTT |
| | | TCCATTGACGTCAATGGGTGGAGTATTTACGGTAAACTGCCCACTTGGCAGTACATCAAGTGT |
| | | ATCATATGCCAAGTACGCCCCCTATTGACGTCAATGACGGTAAATGGCCCGCCTGGCATTATG |
| | | CCCAGTACATGACCTTATGGGACTTTCCTACTTGGCAGTACATCTACGTATTAGTCATCGCTA |
| | | TTACCATGGTCGAGGTGAGCCCCACGTTCTGCTTCACTCTCCCCATCTCCCCCCCCTCCCCAC |
| | | CCCCAATTTTGTATTTATTTATTTTTTAATTATTTTGTGCAGCGATGGGGGGGGGGGGGGGGG |
| | | GGGGGCGCGCGCCAGGCGGGGCGGGGCGGGGCGAGGGGCGGGGCGGGGCGAGGCGGAGAGGTG |
| | | CGGCGGCAGCCAATCAGAGCGGCGCGCTCCGAAAGTTTCCTTTTATGGCGAGGCGGCGGCGGC |
| | | GGCGGCCCTATAAAAAGCGAAGCGCGCGGCGGGCGGGAGTCGCTGCGCGCTGCCTTCGCCCCG |
| | | TGCCCCGCTCCGCCGCCGCCTCGCGCCGCCCGCCCCGGCTCTGACTGACCGCGTTACTCCCAC |
| | | AGGTGAGCGGGCGGGACGGCCCTTCTCCTCCGGGCTGTAATTAGCGCTTGGTTTAATGACGGC |
| | | TTGTTTCTTTTCTGTGGCTGCGTGAAAGCCTTGAGGGGCTCCGGGAGGGCCCTTTGTGCGGGG |
| | | GGAGCGGCTCGGGGGGTGCGTGCGTGTGTGTGTGCGTGGGGAGCGCCGCGTGCGGCTCCGCGC |
| | | TGCCCGGCGGCTGTGAGCGCTGCGGGCGCGGCGCGGGGCTTTGTGCGCTCCGCAGTGTGCGCG |
| | | AGGGGAGCGCGGCCGGGGGCGGTGCCCCGCGGTGCGGGGGGGGCTGCGAGGGGAACAAAGGCT |
| | | GCGTGCGGGGTGTGTGCGTGGGGGGGTGAGCAGGGGGTGTGGGCGCGTCGGTCGGGCTGCAAC |
| | | CCCCCCTGCACCCCCCTCCCCGAGTTGCTGAGCACGGCCCGGCTTCGGGTGCGGGGCTCCGTA |
| | | CGGGGCGTGGCGCGGGGCTCGCCGTGCCGGGCGGGGGGTGGCGGCAGGTGGGGGTGCCGGGCG |
| | | GGGCGGGGCCGCCTCGGGCCGGGGAGGGCTCGGGGGAGGGGCGCGGCGGCCCCCGGAGCGCCG |
| | | GCGGCTGTCGAGGCGCGGCGAGCCGCAGCCATTGCCTTTTATGGTAATCGTGCGAGAGGGCGC |
| | | AGGGACTTCCTTTGTCCCAAATCTGTGCGGAGCCGAAATCTGGGAGGCGCCGCCGCACCCCCT |
| | | CTAGCGGGCGCGGGGCGAAGCGGTGCGGCGCCGGCAGGAAGGAAATGGGCGGGGAGGGCCTTC |
| | | GTGCGTCGCCGCGCCGCCGTCCCCTTCTCCCTCTCCAGCCTCGGGGCTGTCCGCGGGGGGACG |
| | | GCTGCCTTCGGGGGGGACGGGGCAGGGCGGGGTTCGGCTTCTGGCGTGTGACCGGCGGCTCTA |
| | | GAGCCTCTGCTAACCATGTTCATGCCTTCTTCTTTTTCCTACAGCTCCTGGGCAACGTGCTGG |
| | | TTATTGTGCTGTCTCATCATTTTGGCAAAGAATTC |
| CB6 | 1048 | CTAGTCGACATTGATTATTGACTAGTTATTAATAGTAATCAATTACGGGGTCATTAGTTCATA |
| Promoter | | GCCCATATATGGAGTTCCGCGTTACATAACTTACGGTAAATGGCCCGCCTGGCTGACCGCCCA |
| | | ACGACCCCCGCCCATTGACGTCAATAATGACGTATGTTCCCATAGTAACGCCAATAGGGACTT |
| | | TCCATTGACGTCAATGGGTGGAGTATTTACGGTAAACTGCCCACTTGGCAGTACATCAAGTGT |
| | | ATCATATGCCAAGTACGCCCCCTATTGACGTCAATGACGGTAAATGGCCCGCCTGGCATTATG |
| | | CCCAGTACATGACCTTATGGGACTTTCCTACTTGGCAGTACATCTACGTA |
| CAG | 1049 | CTAGTTATTAATAGTAATCAATTACGGGGTCATTAGTTCATAGCCCATATATGGAGTTCCGCG |
| Promoter | | TTACATAACTTACGGTAAATGGCCCGCCTGGCTGACCGCCCAACGACCCCCGCCCATTGACGT |
| Variant 3 | | CAATAATGACGTATGTTCCCATAGTAACGCCAATAGGGACTTTCCATTGACGTCAATGGGTGG |
| | | AGTATTTACGGTAAACTGCCCACTTGGCAGTACATCAAGTGTATCATATGCCAAGTACGCCCC |
| | | CTATTGACGTCAATGACGGTAAATGGCCCGCCTGGCATTATGCCCAGTACATGACCTTATGGG |
| | | ACTTTCCTACTTGGCAGTACATCTACGTATTAGTCATCGCTATTACCATGGTCGAGGTGAGCC |
| | | CCACGTTCTGCTTCACTCTCCCCATCTCCCCCCCCTCCCCACCCCCAATTTTGTATTTATTTA |
| | | TTTTTTAATTATTTTGTGCAGCGATGGGGGCGGGGGGGGGGGGGGGGCGCGCGCCAGGCGGGG |
| | | CGGGGCGGGGCGAGGGGCGGGGCGGGGCGAGGCGGAGAGGTGCGGCGGCAGCCAATCAGAGCG |
| | | GCGCGCTCCGAAAGTTTCCTTTTATGGCGAGGCGGCGGCGGCGGCGGCCCTATAAAAAGCGAA |
| | | GCGCGCGGCGGGCGGGAGTCGCTGCGCGCTGCCTTCGCCCCGTGCCCCGCTCCGCCGCCGCCT |
| | | CGCGCCGCCCGCCCCGGCTCTGACTGACCGCGTTACTCCCACAGGTGAGCGGGCGGGACGGCC |
| | | CTTCTCCTCCGGGCTGTAATTAGCGCTTGGTTTAATGACGGCTTGTTTCTTTTCTGTGGCTGC |
| | | GTGAAAGCCTTGAGGGGCTCCGGGAGGGCCCTTTGTGCGGGGGGAGCGGCTCGGGGGGTGCGT |
| | | GCGTGTGTGTGTGCGTGGGGAGCGCCGCGTGCGGCTCCGCGCTGCCCGGCGGCTGTGAGCGCT |
| | | GCGGGCGCGGCGCGGGGCTTTGTGCGCTCCGCAGTGTGCGCGAGGGGAGCGCGGCCGGGGGCG |
| | | GTGCCCCGCGGTGCGGGGGGGGCTGCGAGGGGAACAAAGGCTGCGTGCGGGGTGTGTGCGTGG |
| | | GGGGGTGAGCAGGGGGTGTGGGCGCGTCGGTCGGGCTGCAACCCCCCCTGCACCCCCCTCCCC |
| | | GAGTTGCTGAGCACGGCCCGGCTTCGGGTGCGGGGCTCCGTACGGGGCGTGGCGCGGGGCTCG |
| | | CCGTGCCGGGCGGGGGGTGGCGGCAGGTGGGGGTGCCGGGCGGGGCGGGGCCGCCTCGGGCCG |
| | | GGGAGGGCTCGGGGGAGGGGCGCGGCGGCCCCCGGAGCGCCGGCGGCTGTCGAGGCGCGGCGA |
| | | GCCGCAGCCATTGCCTTTTATGGTAATCGTGCGAGAGGGCGCAGGGACTTCCTTTGTCCCAAA |
| | | TCTGTGCGGAGCCGAAATCTGGGAGGCGCCGCCGCACCCCCTCTAGCGGGCGCGGGGCGAAGC |
| | | GGTGCGGCGCCGGCAGGAAGGAAATGGGCGGGGAGGGCCTTCGTGCGTCGCCGCGCCGCCGTC |
| | | CCCTTCTCCCTCTCCAGCCTCGGGGCTGTCCGCGGGGGGACGGCTGCCTTCGGGGGGGACGGG |
| | | GCAGGGCGGGGTTCGGCTTCTGGCGTGTGACCGGCGGCTCTAGAGCCTCTGCTAACCATGTTC |
| | | ATGCCTTCTTCTTTTTCCTACAGCTCCTGGGCAACGTGCTGGTTATTGTGCTGTCTCATCATT |
| | | TTGGCAAAGAATT |
| CMVie | 1050 | GACATTGATTATTGACTAGTTATTAATAGTAATCAATTACGGGGTCATTAGTTCATAGCCCAT |
| Promoter | | ATATGGAGTTCCGCGTTACATAACTTACGGTAAATGGCCCGCCTGGCTGACCGCCCAACGACC |
| Variant 3 | | CCCGCCCATTGACGTCAATAATGACGTATGTTCCCATAGTAACGCCAATAGGGACTTTCCATT |
| | | GACGTCAATGGGTGGAGTATTTACGGTAAACTGCCCACTTGGCAGTACATCAAGTGTATCATA |
| | | TGCCAAGTACGCCCCCTATTGACGTCAATGACGGTAAATGGCCCGCCTGGCATTATGCCCAGT |
| | | ACATGACCTTATGGGACTTTCCTACTTGGCAGTACATCTACGTATTAGTCATCGCTATTACCA |
| | | TG |

In some embodiments, the promoter comprises the nucleotide sequence of any one of SEQ ID NOs: 1039-1050, or a sequence with at least 70%, 75%, 80%, 85%, 90%, 95% or 99% sequence identity thereto. In some embodiments, the promoter comprises the nucleotide sequence of SEQ ID NO: 1039 or a sequence with at least 70%, 75%, 80%, 85%, 90%, 95% or 99% sequence identity thereto. In some embodiments, the promoter comprises the nucleotide sequence of SEQ ID NO: 1040, or a sequence with at least 70%, 75%, 80%, 85%, 90%, 95% or 99% sequence identity thereto. In some embodiments, the promoter comprises the nucleotide sequence of SEQ ID NO: 1041, or a sequence with at least 70%, 75%, 80%, 85%, 90%, 95% or 99% sequence identity thereto. In some embodiments, the promoter comprises the nucleotide sequence of SEQ ID NO: 1042, or a sequence with at least 70%, 75%, 80%, 85%, 90%, 95% or 99% sequence identity thereto. In some embodiments, the promoter comprises the nucleotide sequence of SEQ ID NO: 1043, or a sequence with at least 70%, 75%, 80%, 85%, 90%, 95% or 99% sequence identity thereto. In some embodiments, the promoter comprises the nucleotide sequence of SEQ ID NO: 1044, or a sequence with at least 70%, 75%, 80%, 85%, 90%, 95% or 99% sequence identity thereto. In some embodiments, the promoter comprises the nucleotide sequence of SEQ ID NO: 1050, or a sequence with at least 70%, 75%, 80%, 85%, 90%, 95% or 99% sequence identity thereto.

In some embodiments, the CAG promoter comprises the nucleotide sequence of SEQ ID NO: 1039 or nucleotide sequence with at least 70%, 75%, 80%, 85%, 90%, 95%, or 99% sequence identity thereto. In some embodiments, the CBA promoter (e.g., a minimal CBA promoter) comprises the nucleotide sequence of SEQ ID NO: 1041 or nucleotide sequence with at least 70%, 75%, 80%, 85%, 90%, 95%, or 99% sequence identity thereto. In some embodiments, the CB promoter comprises the nucleotide sequence of SEQ ID NO: 1042 or nucleotide sequence with at least 70%, 75%, 80%, 85%, 90%, 95%, or 99% sequence identity thereto. In some embodiments, the GFAP promoter comprises the nucleotide sequence of SEQ ID NO: 1044 or nucleotide sequence with at least 70%, 75%, 80%, 85%, 90%, 95%, or 99% sequence identity thereto. In some embodiments, the synapsin promoter comprises the nucleotide sequence of SEQ ID NO: 1045 or nucleotide sequence with at least 70%, 75%, 80%, 85%, 90%, 95%, or 99% sequence identity thereto. In some embodiments, the CMV(IE) promoter comprises the nucleotide sequence of SEQ ID NO: 1050 or nucleotide sequence with at least 70%, 75%, 80%, 85%, 90%, 95%, or 99% sequence identity thereto. In some embodiments, the CMV(ie) enhancer comprises e nucleotide sequence of SEQ ID NO: 1040 or nucleotide sequence with at least 70%, 75%, 80%, 85%, 90%, 95%, or 99% sequence identity thereto.

In some embodiments, the viral genome comprises more than one promoter sequence region. In some embodiments, the viral genome comprises two promoter sequence regions. In some embodiments, the viral genome comprises three promoter sequence regions.

### Viral Genome Component: Untranslated Regions (UTRs)

In some embodiments, the viral genome comprises an untranslated region (UTR). In some embodiments, a wild type UTR of a gene are transcribed but not translated. In some embodiments, the 5′ UTR starts at the transcription start site and ends at the start codon and the 3′ UTR starts immediately following the stop codon and continues until the termination signal for transcription.

In some embodiments, a UTR comprises a feature found in abundantly expressed genes of specific target organs to enhance the stability and protein production. As a non-limiting example, a 5′ UTR from mRNA normally expressed in the liver (e.g., albumin, serum amyloid A, Apolipoprotein A/B/E, transferrin, alpha fetoprotein, erythropoietin, or Factor VIII) may be used in the viral genome of an AAV particle described herein to enhance expression in hepatic cell lines or liver.

In some embodiments, the viral genome comprises a 5′UTR, e.g., a wild-type (e.g., naturally occurring) 5′UTR or a recombinant (e.g., non-naturally occurring) 5′UTR. In some embodiments, a 5′ UTR comprises a feature which plays a role in translation initiation. In some embodiments, a UTR, e.g., a 5′ UTR, comprises a Kozak sequence. In some embodiments, a Kozak sequence is involved in the process by which the ribosome initiates translation of many genes. In some embodiments, a Kozak sequence has the consensus sequence of CCR(A/G)CCAUGG, where R is a purine (adenine or guanine) three bases upstream of the start codon (ATG), which is followed by another ‘G’. In some embodiments, a Kozak sequence comprises the nucleotide sequence of GAGGAGCCACC (SEQ ID NO: 1089) or a nucleotide sequence with at least 95-99% sequence identity thereto. In some embodiments, a Kozak sequence comprises the nucleotide sequence of GCCGCCACCATG (SEQ ID NO: 1079), or a nucleotide sequence with at least 95-99% sequence identity thereto. In some embodiments, a viral genome comprises a 5′UTR comprising a Kozak sequence. In some embodiments, a viral genome comprises a 5′UTR that does not comprise a Kozak sequence.

In some embodiments, the viral genome comprises a 3′UTR, e.g., a wild-type (e.g., naturally occurring) 3′UTR or a recombinant (e.g., non-naturally occurring) 3′UTR. In some embodiments, a 3′ UTR comprises an element that modulates, e.g., increases or decreases, stability of a nucleic acid. In some embodiments, a 3′ UTR comprises stretches of Adenosines and Uridines embedded therein, e.g., an AU rich signature. These AU rich signatures are generally prevalent in genes with high rates of turnover and are described, e.g., in Chen et al, 1995, the contents of which are herein incorporated by reference in its entirety. In some embodiments, an AR rich signature comprises an AU rich element (ARE). In some embodiments, a 3′UTR comprises an ARE chosen from a class I ARE (e.g., c-Myc and MyoD), a class II ARE (e.g., GM-CSF and TNF-α), a class III ARE (e.g., c-Jun and Myogenin), or combination thereto. In some embodiments, a class I ARE comprises several dispersed copies of an AUUUA motif within U-rich regions. In some embodiments, a class II ARE comprises two or more overlapping UUAUUUA(U/AXU/A) nonamers. In some embodiments, a class III ARE comprises U rich regions and/or do not contain an AUUUA motif. In some embodiments, an ARE destabilizes the messenger.

In some embodiments, a 3′UTR comprises a binding site for a protein member of the ELAV family. In some embodiments, a 3′ UTR comprises a binding site for an HuR protein. In some embodiments, an HuR protein binds to an ARE of any one of classes I-III and/or increases the stability of mRNA. Without wishing to be bound by theory, it is believed in some embodiments, that a 3′UTR comprising an HuR specific binding sites will lead to HuR binding and, stabilization of a message in vivo.

In some embodiments, the 3′ UTR of the viral genome comprises an oligo(dT) sequence for templated addition of a poly-A tail.

In some embodiments, the viral genome comprises a miRNA seed, binding site and/or full sequence. Generally, microRNAs (or miRNA or miR) are 19-25 nucleotide noncoding RNAs that bind to the sites of nucleic acid targets and down-regulate gene expression either by reducing nucleic acid molecule stability or by inhibiting translation. In some embodiments, the microRNA sequence comprises a seed region, e.g., a sequence in the region of positions 2-8 of the mature microRNA, which sequence has perfect Watson-Crick complementarity to the miRNA target sequence of the nucleic acid. In some embodiments, the viral genome may be engineered to include, alter or remove at least one miRNA binding site, sequence, or seed region.

In some embodiments, a UTR from any gene known in the art may be incorporated into the AAV particle viral genome described herein. These UTRs, or portions thereof, may be placed in the same orientation as in the gene from which they were selected, or they may be altered in orientation or location. In some embodiments, the UTR used in the viral genome may be inverted, shortened, lengthened, made with one or more other 5′ UTRs or 3′ UTRs known in the art. In some embodiments, an altered UTR, comprises a UTR has been changed in some way in relation to a reference sequence. For example, a 3′ or 5′ UTR may be altered relative to a wild type or native UTR by the change in orientation or location as taught above or may be altered by the inclusion of additional nucleotides, deletion of nucleotides, swapping or transposition of nucleotides. In some embodiments, the viral genome comprises an artificial UTR, e.g., a UTR that is not a variant of a wild-type, e.g., a naturally occurring, UTR. In some embodiments, the viral genome comprises a UTR selected from a family of transcripts whose proteins share a common function, structure, feature or property.

### Viral Genome Component: MiR Binding Site

Tissue- or cell-specific expression of the AAV viral particles of the invention can be enhanced by introducing tissue- or cell-specific regulatory sequences, e.g., promoters, enhancers, microRNA binding sites, e.g., a detargeting site. Without wishing to be bound by theory, it is believed that an encoded miR binding site can modulate, e.g., prevent, suppress, or otherwise inhibit, the expression of a gene of interest on the viral genome of the invention, based on the expression of the corresponding endogenous microRNA (miRNA) or a corresponding controlled exogenous miRNA in a tissue or cell, e.g., a non-targeting cell or tissue. In some embodiments, a miR binding site modulates, e.g., reduces, expression of the payload encoded by a viral genome of an AAV particle described herein in a cell or tissue where the corresponding mRNA is expressed.

In some embodiments, the viral genome of an AAV particle described herein comprises a nucleotide sequence encoding a microRNA binding site, e.g., a detargeting site. In some embodiments, the viral genome of an AAV particle described herein comprises a nucleotide sequence encoding a miR binding site, a microRNA binding site series (miR BSs), or a reverse complement thereof.

In some embodiments, the nucleotide sequence encoding the miR binding site series or the miR binding site is located in the 3′-UTR region of the viral genome (e.g., 3′ relative to the nucleotide sequence encoding a payload), e.g., before the polyA sequence, 5′-UTR region of the viral genome (e.g., 5′ relative to the nucleotide sequence encoding a payload), or both.

In some embodiments, the encoded miR binding site series comprise at least 1-5 copies, e.g., at least 1-3, 2-4, 3-5, 1, 2, 3, 4, 5 or more copies of a miR binding site (miR BS). In some embodiments, all copies are identical, e.g., comprise the same miR binding site. In some embodiments, the miR binding sites within the encoded miR binding site series are continuous and not separated by a spacer. In some embodiments, the miR binding sites within an encoded miR binding site series are separated by a spacer, e.g., a non-coding sequence. In some embodiments, the spacer is about 1 to 6 nucleotides or about 5 to 10 nucleotides, e.g., about 7-8 nucleotides, nucleotides in length. In some embodiments, the spacer coding sequence or reverse complement thereof comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, but no more than four modifications of GATAGTTA.

In some embodiments, the encoded miR binding site series comprise at least 1-5 copies, e.g., at least 1-3, 2-4, 3-5, 1, 2, 3, 4, 5 or more copies of a miR binding site (miR BS). In some embodiments, at least 1, 2, 3, 4, 5, or all of the copies are different, e.g., comprise a different miR binding site. In some embodiments, the miR binding sites within the encoded miR binding site series are continuous and not separated by a spacer. In some embodiments, the miR binding sites within an encoded miR binding site series are separated by a spacer, e.g., a non-coding sequence. In some embodiments, the spacer is about 1 to 6 nucleotides or about 5 to 10 nucleotides, e.g., about 7-8 nucleotides, in length. In some embodiments, the spacer comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, but no more than four modifications of GATAGTTA.

In some embodiments, the encoded miR binding site is substantially identical (e.g., at least 70%, 75%, 80%, 85%, 90%, 95%, 99% or 100% identical), to the miR in the host cell. In some embodiments, the encoded miR binding site comprises at least 1, 2, 3, 4, or 5 mismatches or no more than 6, 7, 8, 9, or 10 mismatches to a miR in the host cell. In some embodiments, the mismatched nucleotides are contiguous. In some embodiments, the mismatched nucleotides are non-contiguous. In some embodiments, the mismatched nucleotides occur outside the seed region-binding sequence of the miR binding site, such as at one or both ends of the miR binding site. In some embodiments, the miR binding site is 100% identical to the miR in the host cell.

In some embodiments, the nucleotide sequence encoding the miR binding site is substantially complimentary (e.g., at least 70%, 75%, 80%, 85%, 90%, 95%, 99% or 100% complimentary), to the miR in the host cell. In some embodiments, to complementary sequence of the nucleotide sequence encoding the miR binding site comprises at least 1, 2, 3, 4, or 5 mismatches or no more than 6, 7, 8, 9, or 10 mismatches to a miR in the host cell. In some embodiments, the mismatched nucleotides are contiguous. In some embodiments, the mismatched nucleotides are non-contiguous. In some embodiments, the mismatched nucleotides occur outside the seed region-binding sequence of the miR binding site, such as at one or both ends of the miR binding site. In some embodiments, the encoded miR binding site is 100% complimentary to the miR in the host cell.

In some embodiments, an encoded miR binding site or sequence region is at least about 10 to about 125 nucleotides in length, e.g., at least about 10 to 50 nucleotides, 10 to 100 nucleotides, 50 to 100 nucleotides, 50 to 125 nucleotides, or 100 to 125 nucleotides in length. In some embodiments, an encoded miR binding site or sequence region is at least about 7 to about 28 nucleotides in length, e.g., at least about 8-28 nucleotides, 7-28 nucleotides, 8-18 nucleotides, 12-28 nucleotides, 20-26 nucleotides, 22 nucleotides, 24 nucleotides, or 26 nucleotides in length, and optionally comprises at least one consecutive region (e.g., 7 or 8 nucleotides) complementary (e.g., fully or partially complementary) to the seed sequence of a miRNA (e.g., a miR122, a miR142, a miR183).

In some embodiments, the encoded miR binding site is complementary (e.g., fully or partially complementary) to a miR expressed in liver or hepatocytes, such as miR122. In some embodiments, the encoded miR binding site or encoded miR binding site series comprises a miR122 binding site sequence. In some embodiments, the encoded miR122 binding site comprises the nucleotide sequence of ACAAACACCATTGTCACACTCCA (SEQ ID NO: 1029), or a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 1029, e.g., wherein the modification can result in a mismatch between the encoded miR binding site and the corresponding miRNA. In some embodiments, the viral genome comprises at least 2, 3, 4, or 5 copies of the encoded miR122 binding site, e.g., an encoded miR122 binding site series, optionally wherein the encoded miR122 binding site series comprises the nucleotide sequence of: ACAAACACCATTGTCACACTCCACACAAACACCATTGTCACACTCCACACAAACACCATTGTCACACT CCA (SEQ ID NO: 1030), or a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 1030, e.g., wherein the modification can result in a mismatch between the encoded miR binding site and the corresponding miRNA. In some embodiments, at least two of the encoded miR122 binding sites are connected directly, e.g., without a spacer. In other embodiments, at least two of the encoded miR122 binding sites are separated by a spacer, e.g., 1, 2, 3, 4, 5, 6, 7, 8, 9, or 10 nucleotides in length, which is located between two or more consecutive encoded miR122 binding site sequences. In embodiments, the spacer is about 1 to 6 nucleotides or about 5 to 10 nucleotides, e.g., about 7-8, in length. In some embodiments, the spacer coding sequence or reverse complement thereof comprises one or more of (i) GGAT; (ii) CACGTG; (iii) GCATGC, or a repeat of one or more of (i)-(iii). In some embodiments, an encoded miR binding site series comprises at least 3-5 copies (e.g., 4 copies) of a miR122 binding site, with or without a spacer, wherein the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA, or a nucleotide sequence having at least one, two, or three modifications, but no more than four modifications of GATAGTTA.

In some embodiments, the encoded miR binding site is complementary (e.g., fully or partially complementary) to a miR expressed in the heart. In embodiments, the encoded miR binding site or encoded miR binding site series comprises a miR-1 binding site. In some embodiments, the encoded miR-1 binding site comprises the nucleotide sequence of ATACATACTrCTITACATTCCA (SEQ ID NO: 4679), a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications e.g., substitutions (e.g., conservative substitutions), insertions, or deletions, but no more than ten modifications, e.g., substitutions (e.g., conservative substitutions), insertions, or deletions, to SEQ ID NO: 4679, e.g., wherein the modification can result in a mismatch between the encoded miR binding site and the corresponding miRNA. In some embodiments, the viral genome comprises at least 2, 3, 4, or 5 copies of the encoded miR-1 binding site, e.g., an encoded miR-1 binding site series. In some embodiments, the at least 2, 3, 4, or 5 copies (e.g., 2 or 3 copies) of the encoded miR-1 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA.

In some embodiments, the encoded miR binding site is complementary (e.g., fully or partially complementary) to a miR expressed in hematopoietic lineage, including immune cells (e.g., antigen presenting cells or APC, including dendritic cells (DCs), macrophages, and B-lymphocytes). In some embodiments, the encoded miR binding site complementary to a miR expressed in hematopoietic lineage comprises a nucleotide sequence disclosed, e.g., in US 2018/0066279, the contents of which are incorporated by reference herein in its entirety.

In embodiments, the encoded miR binding site or encoded miR binding site series comprises a miR-142-3p binding site sequence. In some embodiments, the encoded miR-142-3p binding site comprises the nucleotide sequence of TCCATAAAGTAGGAAACACTACA (SEQ ID NO: 1031), a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 1031, e.g., wherein the modification can result in a mismatch between the encoded miR binding site and the corresponding miRNA. In some embodiments, the viral genome comprises at least 2, 3, 4, or 5 copies of the encoded miR-142-3p binding site, e.g., an encoded miR-142-3p binding site series. In some embodiments, the at least 2, 3, 4, or 5 copies (e.g., 2 or 3 copies) of the encoded miR-142-3p binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, but no more than four modifications of GATAGTTA.

In some embodiments, the encoded miR binding site is complementary (e.g., fully complementary or partially complementary) to a miR expressed in a DRG (dorsal root ganglion) neuron, e.g., a miR183, a miR182, and/or miR96 binding site. In some embodiments, the encoded miR binding site is complementary to a miR expressed in expressed in a DRG neuron comprises a nucleotide sequence disclosed, e.g., in WO2020/132455, the contents of which are incorporated by reference herein in its entirety.

In some embodiments, the encoded miR binding site or encoded miR binding site series comprises a miR183 binding site sequence. In some embodiments, the encoded miR183 binding site comprises the nucleotide sequence of AGTGAATTCTACCAGTGCCATA (SEQ ID NO: 1032), or a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 1032, e.g., wherein the modification can result in a mismatch between the encoded miR binding site and the corresponding miRNA. In some embodiments, the sequence complementary to the seed sequence corresponds to the double underlined of the encoded miR-183 binding site sequence. In some embodiments, the viral genome comprises at least comprises at least 2, 3, 4, or 5 copies (e.g., at least 2 or 3 copies) of the encoded miR183 binding site, e.g. an encoded miR183 binding site. In some embodiments, the at least 2, 3, 4, or 5 copies (e.g., 2 or 3 copies) of the encoded miR183 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA, or a nucleotide sequence having at least one, two, or three modifications, but no more than four modifications of GATAGTTA. In some embodiments, the spacer sequence comprises one or more of (i) GGAT; (ii) CACGTG; (iii) GCATGC, or a repeat of one or more of (i)-(iii).

In some embodiments, the encoded miR binding site or the encoded miR binding site series comprises a miR182 binding site sequence. In some embodiments, the encoded miR182 binding site comprises, the nucleotide sequence of AGTGTGAGTTCTACCATTGCCAAA (SEQ ID NO: 1033), a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 1033, e.g., wherein the modification can result in a mismatch between the encoded miR binding site and the corresponding miRNA. In some embodiments, the viral genome comprises at least 2, 3, 4, or 5 copies of the encoded miR182 binding site, e.g., an encoded miR182 binding site series. In some embodiments, the at least 2, 3, 4, or 5 copies (e.g., 2 or 3 copies) of the encoded miR182 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA, or a nucleotide sequence having at least one, two, or three modifications, but no more than four modifications of GATAGTTA. In some embodiments, the spacer sequence comprises one or more of (i) GGAT; (ii) CACGTG; (iii) GCATGC, or a repeat of one or more of (i)-(iii).

In certain embodiments, the encoded miR binding site or the encoded miR binding site series comprises a miR96 binding site sequence. In some embodiments, the encoded miR96 binding site comprises the nucleotide sequence of AGCAAAAATGTGCTAGTGCCAAA (SEQ ID NO: 1034), a sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 1034, e.g., wherein the modification can result in a mismatch between the encoded miR binding site and the corresponding miRNA. In some embodiments, the viral genome comprises at least 3, 4, or 5 copies of the encoded miR96 binding site, e.g., an encoded miR96 binding site series. In some embodiments, an encoded miR binding site series comprises at least 3-5 copies (e.g., 4 copies) of a miR96 binding site, with or without a spacer, wherein the spacer is at least about 5 to 10 nucleotides, e.g., about 7-8, in length.

In some embodiments, the viral genome comprises at least 2, 3, 4, or 5 copies of the encoded miR96 binding site, e.g., an encoded miR96 binding site series. In some embodiments, the at least 2, 3, 4, or 5 copies (e.g., 2 or 3 copies) of the encoded miR96 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA, or a nucleotide sequence having at least one, two, or three modifications, but no more than four modifications of GATAGTTA. In some embodiments, the spacer sequence comprises one or more of (i) GGAT; (ii) CACGTG; (iii) GCATGC, or a repeat of one or more of (i)-(iii).

In some embodiments, the encoded miR binding site series comprises a miR122 binding site, a miR142 binding site, a miR183 binding site, a miR182 binding site, a miR 96 binding site, or a combination thereof. In some embodiments, the encoded miR binding site series comprises at least 2, 3, 4, or 5 copies of a miR122 binding site, a miR1 binding site, a miR142 binding site, a miR183 binding site, a miR182 binding site, a miR 96 binding site, or a combination thereof. In some embodiments, at least two of the encoded miR binding sites are connected directly, e.g., without a spacer. In other embodiments, at least two of the encoded miR binding sites are separated by a spacer, e.g., 1, 2, 3, 4, 5, 6, 7, 8, 9, or 10 nucleotides in length, which is located between two or more consecutive encoded miR binding site sequences. In embodiments, the spacer is at least about 5 to 10 nucleotides, e.g., about 7-8 nucleotides or about 8 nucleotides, in length. In some embodiments, the spacer coding sequence or reverse complement thereof comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, but no more than four modifications of GATAGTTA.

In some embodiments, an encoded miR binding site series comprises at least 2-5 copies (e.g., 2 or 3 copies) of a combination of at least two, three, four, five, six, or all of a miR122 binding site, a mir1 binding site, a miR142 binding site, a miR183 binding site, a miR182 binding site, a miR96 binding site, wherein each of the miR binding sites within the series are continuous (e.g., not separated by a spacer) or are separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA, or a nucleotide sequence having at least one, two, or three modifications, but no more than four modifications of GATAGTTA.

### Viral Genome Component: Exon Sequence Region

In some embodiments, the viral genome may comprise at least one exon sequence region. In some embodiments, the viral genome comprises at least 2, at least 3, at least 4, or at least 5 exon regions. In some embodiments, the viral genome comprises two Exon sequence regions. In some embodiments, the viral genome comprises three Exon sequence regions. In some embodiments, the viral genome comprises four Exon sequence regions. In some embodiments, the viral genome comprises more than four Exon sequence regions.

In some embodiments, the exon region is provided in Table 12. In some embodiments, the exon region comprises the nucleotide sequence of any one of SEQ ID NOs: 1051-1055, or a nucleotide sequence with at least 70%, 75%, 80%, 85%, 90%, 95%, or 99% sequence identity thereto.

TABLE 12
Exon Sequence Regions

| | SEQ | |
|---|---|---|
| | ID | |
| Description | NO | Sequence |
| ie1 exon 1-variant 1 | 1051 | TCAGATCGCCTGGAGACGCCATCCACGCTGTTTTGACCTCCATAGAAGA |
| | | CACCGGGACCGATCCAGCCTCCGCGGATTCGAATCCCGGCCGGGAACGG |
| | | TGCATTGGAACGCGGATTCCCCGTGCCAAGAGTGAC |
| ie1 exon 1-variant 2 | 1052 | TTGACCTCCATAGAAGACACCGGGACCGATCCAGCCTCCGCGGATTCGA |
| | | ATCCCGGCCGGGAACGGTGCATTGGAACGCGGATTCCCCGTGCCAAGAG |
| | | TGAC |
| ie1 exon 1-variant 3 | 1053 | ATTCGAATCCCGGCCGGGAACGGTGCATTGGAACGCGGATTCCCCGTGC |
| | | CAAGAGTGAC |
| Human beta globin | 1054 | CTCCTGGGCAACGTGCTGGTCTGTGTGCTGGCCCATCACTTTGGCAAAG |
| (HBG) exon3 | | AATT |
| rabbit beta globin | 1055 | CTCCTGGGCAACGTGCTGGTTATTGTGCTGTCTCATCATTTTGGCAAAG |
| (RBG) exon 3 | | AATTC |

### Viral Genome Component: Intron Sequence Region

In some embodiments, the viral genome comprises at least one element to enhance the expression of a transgene encoding a payload. In some embodiments, an element that enhances expression of a transgene comprises an introns or functional variant thereof. In some embodiments, the viral genome comprises an intron or functional variant thereof. In some embodiments, the viral genome comprises at least two intron regions, e.g., at least 2 intron regions, at least 3 intron regions, at least 4 intron regions, or 5 or more intron regions.

In some embodiments, the viral genome comprises an intron chosen from a MVM intron (67-97 bps), an F.IX truncated intron 1 (300 bps), an β-globin SD/immunoglobulin heavy chain splice acceptor intron (250 bps), an adenovirus splice donor/immunoglobin splice acceptor intron (500 bps), SV40 late splice donor/splice acceptor intron (19S/16S) (180 bps), or a hybrid adenovirus splice donor/IgG splice acceptor intron (230 bps). In some embodiments, the viral genome comprises a human beta-globin intron region.

In some embodiments, the viral genome comprises an intron region provided in Table 13.

TABLE 13
Intron Sequence Regions

| Sequence | SEQ ID | |
|---|---|---|
| Region Name | NO | Sequence |
| ie1 intron 1- | 1056 | GTAAGTACCGCCTATAGAGTCTATAGGCCCAC |
| Variant 1 | | |
| ie1 intron 1- | 1057 | GTAAGTACCGCCTAT |
| Variant 2 | | |
| HBG intron 2- | 1058 | AAAAAATGCTTTCTTCTTTTAATATACTTTTTTGTTTATCTTATTTCTAATA |
| Variant 1 | | CTTTCCCTAATCTCTTTCTTTCAGGGCAATAATGATACAATGTATCATGCCT |
| | | CTTTGCACCATTCTAAAGAATAACAGTGATAATTTCTGGGTTAAGGCAATAG |
| | | CAATATTTCTGCATATAAATATTTCTGCATATAAATTGTAACTGATGTAAGA |
| | | GGTTTCATATTGCTAATAGCAGCTACAATCCAGCTACCATTCTGCTTTTATT |
| | | TTATGGTTGGGATAAGGCTGGATTATTCTGAGTCCAAGCTAGGCCCTTTTGC |
| | | TAATCATGTTCATACCTCTTATCTTCCTCCCACAG |
| HBG intron 2- | 1059 | TCTGCATATAAATTGTAACTGATGTAAGAGGTTTCATATTGCTAATAGCAGC |
| Variant 2 | | TACAATCCAGCTACCATTCTGCTTTTATTTTATGGTTGGGATAAGGCTGGAT |
| | | TATTCTGAGTCCAAGCTAGGCCCTTTTGCTAATCATGTTCATACCTCTTATC |
| | | TTCCTCCCACAG |
| HBG intron 2- | 1060 | AAAAAATGCTTTCTTCTTTTAATATACTTTTCTTTTGCTAATCATGTTCATA |
| Variant 3 | | CCTCTTATCTTCCTCCCACAG |
| HBG intron 2- | 1061 | AGGCTGGATTATTCTGAGTCCAAGCTAGGCCCTTTTGCTAATCATGTTCATA |
| Variant 4 | | CCTCTTATCTTCCTCCCACAG |
| HBG intron 2- | 1062 | AAAAAATGCTTTCTTCTTTTCAAGCTAGGCCCTTTTGCTAATCATGTTCATA |
| Variant 5 | | CCTCTTATCTTCCTCCCACAG |
| HBG intron 2- | 1063 | CAAGCTAGGCCCTTTTGCTAATCATGTTCATACCTCTTATCTTCCTCCCACA |
| Variant 6 | | G |
| SV40 intron | 1064 | GAACTGAAAAACCAGAAAGTTAACTGGTAAGTTTAGTCTTTTTGTCTTTTAT |
| | | TTCAGGTCCCGGATCCGGTGGTGGTGCAAATCAAAGAACTGCTCCTCAGTGG |
| | | ATGTTGCCTTTACTTCTAGGCCTGTACGGAAGTGTTACTTCTGCTCTAAAAG |
| | | CTGCGGAATTGTACCC |
| Chimeric intron | 1065 | GGAGTCGCTGCGCGCTGCCTTCGCCCCGTGCCCCGCTCCGCCGCCGCCTCGC |
| | | GCCGCCCGCCCCGGCTCTGACTGACCGCGTTACTCCCACAGGTGAGCGGGCG |
| | | GGACGGCCCTTCTCCTCCGGGCTGTAATTAGCGCTTGGTTTAATGACGGCTT |
| | | GTTTCTTTTCTGTGGCTGCGTGAAAGCCTTGAGGGGCTCCGGGAGGGCCCTT |
| | | TGTGCGGGGGGAGCGGCTCGGGGGGTGCGTGCGTGTGTGTGTGCGTGGGGAG |
| | | CGCCGCGTGCGGCTCCGCGCTGCCCGGCGGCTGTGAGCGCTGCGGGCGCGGC |
| | | GCGGGGCTTTGTGCGCTCCGCAGTGTGCGCGAGGGGAGCGCGGCCGGGGGCG |
| | | GTGCCCCGCGGTGCGGGGGGGGCTGCGAGGGGAACAAAGGCTGCGTGCGGGG |
| | | TGTGTGCGTGGGGGGGTGAGCAGGGGGTGTGGGCGCGTCGGTCGGGCTGCAA |
| | | CCCCCCCTGCACCCCCCTCCCCGAGTTGCTGAGCACGGCCCGGCTTCGGGTG |
| | | CGGGGCTCCGTACGGGGCGTGGCGCGGGGCTCGCCGTGCCGGGCGGGGGGTG |
| | | GCGGCAGGTGGGGGTGCCGGGCGGGGCGGGGCCGCCTCGGGCCGGGGAGGGC |
| | | TCGGGGGAGGGGCGCGGCGGCCCCCGGAGCGCCGGCGGCTGTCGAGGCGCGG |
| | | CGAGCCGCAGCCATTGCCTTTTATGGTAATCGTGCGAGAGGGCGCAGGGACT |
| | | TCCTTTGTCCCAAATCTGTGCGGAGCCGAAATCTGGGAGGCGCCGCCGCACC |
| | | CCCTCTAGCGGGCGCGGGGCGAAGCGGTGCGGCGCCGGCAGGAAGGAAATGG |
| | | GCGGGGAGGGCCTTCGTGCGTCGCCGCGCCGCCGTCCCCTTCTCCCTCTCCA |
| | | GCCTCGGGGCTGTCCGCGGGGGGACGGCTGCCTTCGGGGGGGACGGGGCAGG |
| | | GCGGGGTTCGGCTTCTGGCGTGTGACCGGCGGCTCTAGAGCCTCTGCTAACC |
| | | ATGTTCATGCCTTCTTCTTTTTCCTACAGCTCCTGGGCAACGTGCTGGTTAT |
| | | TGTGCTGTCTCATCATTTTGGCAAAGAATTCGAG |
| Rbt betaglobin | 1066 | CCTCTGCTAACCATGTTCATGCCTTCTTCTTTTTCCTACAG |
| intron 2 | | |
| HGB intron | 1067 | TCAGATCGCCTGGAGACGCCATCCACGCTGTTTTGACCTCCATAGAAGACAC |
| variant 1 | | CGGGACCGATCCAGCCTCCGCGGATTCGAATCCCGGCCGGGAACGGTGCATT |
| | | GGAACGCGGATTCCCCGTGCCAAGAGTGACGTAAGTACCGCCTATAGAGTCT |
| | | ATAGGCCCACAAAAAATGCTTTCTTCTTTTAATATACTTTTTTGTTTATCTT |
| | | ATTTCTAATACTTTCCCTAATCTCTTTCTTTCAGGGCAATAATGATACAATG |
| | | TATCATGCCTCTTTGCACCATTCTAAAGAATAACAGTGATAATTTCTGGGTT |
| | | AAGGCAATAGCAATATTTCTGCATATAAATATTTCTGCATATAAATTGTAAC |
| | | TGATGTAAGAGGTTTCATATTGCTAATAGCAGCTACAATCCAGCTACCATTC |
| | | TGCTTTTATTTTATGGTTGGGATAAGGCTGGATTATTCTGAGTCCAAGCTAG |
| | | GCCCTTTTGCTAATCATGTTCATACCTCTTATCTTCCTCCCACAGCTCCTGG |
| | | GCAACGTGCTGGTCTGTGTGCTGGCCCATCACTTTGGCAAAGAATT |
| HGB intron | 1068 | ATTCGAATCCCGGCCGGGAACGGTGCATTGGAACGCGGATTCCCCGTGCCAA |
| variant 2 | | GAGTGACGTAAGTACCGCCTATAGAGTCTATAGGCCCACAAAAAATGCTTTC |
| | | TTCTTTTAATATACTTTTTTGTTTATCTTATTTCTAATACTTTCCCTAATCT |
| | | CTTTCTTTCAGGGCAATAATGATACAATGTATCATGCCTCTTTGCACCATTC |
| | | TAAAGAATAACAGTGATAATTTCTGGGTTAAGGCAATAGCAATATTTCTGCA |
| | | TATAAATATTTCTGCATATAAATTGTAACTGATGTAAGAGGTTTCATATTGC |
| | | TAATAGCAGCTACAATCCAGCTACCATTCTGCTTTTATTTTATGGTTGGGAT |
| | | AAGGCTGGATTATTCTGAGTCCAAGCTAGGCCCTTTTGCTAATCATGTTCAT |
| | | ACCTCTTATCTTCCTCCCACAGCTCCTGGGCAACGTGCTGGTCTGTGTGCTG |
| | | GCCCATCACTTTGGCAAAGAATT |
| HGB intron | 1069 | TCAGATCGCCTGGAGACGCCATCCACGCTGTTTTGACCTCCATAGAAGACAC |
| variant 3 | | CGGGACCGATCCAGCCTCCGCGGATTCGAATCCCGGCCGGGAACGGTGCATT |
| | | GGAACGCGGATTCCCCGTGCCAAGAGTGACGTAAGTACCGCCTATAGAGTCT |
| | | ATAGGCCCACTCTGCATATAAATTGTAACTGATGTAAGAGGTTTCATATTGC |
| | | TAATAGCAGCTACAATCCAGCTACCATTCTGCTTTTATTTTATGGTTGGGAT |
| | | AAGGCTGGATTATTCTGAGTCCAAGCTAGGCCCTTTTGCTAATCATGTTCAT |
| | | ACCTCTTATCTTCCTCCCACAGCTCCTGGGCAACGTGCTGGTCTGTGTGCTG |
| | | GCCCATCACTTTGGCAAAGAATT |
| HGB intron | 1070 | TCAGATCGCCTGGAGACGCCATCCACGCTGTTTTGACCTCCATAGAAGACAC |
| variant 4 | | CGGGACCGATCCAGGTAAGTACCGCCTATAGAGTCTATAGGCCCACAAAAAA |
| | | TGCTTTCTTCTTTTAATATACTTTTTTGTTTATCTTATTTCTAATACTTTCC |
| | | CTAATCTCTTTCGCTGGATTATTCTGAGTCCAAGCTAGGCCCTTTTGCTAAT |
| | | CATGTTCATACCTCTTATCTTCCTCCCACAGCTCCTGGGCAACGTGCTGGTC |
| | | TGTGTGCTGGCCCATCACTTTGGCAAAGAATT |

In some embodiments, the viral genome comprises an intron region of any one of SEQ ID NOs: 1056-1070, or a sequence with at least 70%, 75%, 80%, 85%, 90%, 95%, or 99% sequence identity thereto. In some embodiments, the viral genome comprises an intron comprising the nucleotide sequence of SEQ ID NO: 1067, or a nucleotide sequence with at least 70%, 75%, 80%, 85%, 90%, 95%, or 99% sequence identity thereto.

In some embodiments, the viral genome comprises two intron sequence regions. In some embodiments, the viral genome comprises three intron sequence regions. In some embodiments, the viral genome comprises more than three intron sequence regions.

### Viral Genome Component: Polyadenylation Sequence Region

In some embodiments, the viral genome may comprise at least one polyadenylation sequence region. In some embodiments, the viral genome comprises a polyadenylation (referred to herein as poly A, polyA, or poly-A) sequence between the 3′ end of the transgene encoding the payload and the 5′ end of the 3′ITR. In some embodiments, the viral genome comprises two or more polyA sequences. In some embodiments, the viral genome does not comprise a polyA sequence.

TABLE 17
Exemplary Poly-A Signal Sequence Regions

| SEQ ID NO | Sequence |
|---|---|
| 1134 | GATCTTTTTCCCTCTGCCAAAAATTATGGGGACATCATGAAGCCCCTTGAGCATCTGAC |
| | TTCTGGCTAATAAAGGAAATTTATTTTCATTGCAATAGTGTGTTGGAATTTTTTGTGTC |
| | TCTCACTCG |
| 1135 | GGGTGGCATCCCTGTGACCCCTCCCCAGTGCCTCTCCTGGCCCTGGAAGTTGCCACTCC |
| | AGTGCCCACCAGCCTTGTCCTAATAAAATTAAGTTGCATCATTTTGTCTGACTAGGTGT |
| | CCTTCTATAATATTATGGGGTGGAGGGGGGTGGTATGGAGCAAGGGGCAAGTTGGGAAG |
| | ACAACCTGTAGGGCCTGCGGGGTCTATTGGGAACCAAGCTGGAGTGCAGTGGCACAATC |
| | TTGGCTCACTGCAATCTCCGCCTCCTGGGTTCAAGCGATTCTCCTGCCTCAGCCTCCCG |
| | AGTTGTTGGGATTCCAGGCATGCATGACCAGGCTCAGCTAATTTTTGTTTTTTTGGTAG |
| | AGACGGGGTTTCACCATATTGGCCAGGCTGGTCTCCAACTCCTAATCTCAGGTGATCTA |
| | CCCACCTTGGCCTCCCAAATTGCTGGGATTACAGGCGTGAACCACTGCTCCCTTCCCTG |
| | TCCTT |
| 1136 | GAATTCACTCCTCAGGTGCAGGCTGCCTATCAGAAGGTGGTGGCTGGTGTGGCCAATGC |
| | CCTGGCTCACAAATACCACTGAGATCTTTTTCCCTCTGCCAAAAATTATGGGGACATCA |
| | TGAAGCCCCTTGAGCATCTGACTTCTGGCTAATAAAGGAAATTTATTTTCATTGCAATA |
| | GTGTGTTGGAATTTTTTGTGTCTCTCACTCGGAAGGACATATGGGAGGGCAAATCATTT |
| | AAAACATCAGAATGAGTATTTGGTTTAGAGTTTGGCAACATATGCCCATATGCTGGCTG |
| | CCATGAACAAAGGTTGGCTATAAAGAGGTCATCAGTATATGAAACAGCCCCCTGCTGTC |
| | CATTCCTTATTCCATAGAAAAGCCTTGACTTGAGGTTAGATTTTTTTTATATTTTGTTT |
| | TGTGTTATTTTTTTCTTTAACATCCCTAAAATTTTCCTTACATGTTTTACTAGCCAGAT |
| | TTTTCCTCCTCTCCTGACTACTCCCAGTCATAGCTGTCCCTCTTCTCTTATGGAGATCC |
| | CTCGACCTGCAGCCCAAGCTT |

In some embodiments, the polyA signal region comprises the nucleotide sequence of any one of SEQ ID NOs: 1134-1136, or a sequence with at least 70%, 75%, 80%, 85%, 90%, 95%, or 99% sequence identity thereto. In some embodiments, the polyA signal sequence comprises the nucleotide sequence of SEQ ID NO: 1134, or a sequence with at least 70%, 75%, 80%, 85%, 90%, 95%, or 99% sequence identity thereto.

### Viral Genome Component: Payload

In some embodiments, an AAV particle, e.g., an AAV particle for the vectorized delivery of an antibody or fragment thereof described herein (e.g., an anti-tau antibody), comprises a payload. In some embodiments, an AAV particle described herein comprises at least two, at least three, or at least 4 payloads. In some embodiments, an AAV particle, e.g., an AAV particle for the vectorized delivery of an antibody or fragment thereof described herein (e.g., an anti-tau antibody), comprises a nucleic acid comprising a transgene encoding a payload. In some embodiments, the payload comprises an antibody, e.g., an anti-tau antibody. In some embodiments, the payload comprises a secreted protein, an intracellular protein, an extracellular protein, a membrane protein, a structural protein, a functional protein, or a protein, e.g., a mammalian protein, involved in immune system regulation. In some embodiments, a nucleic acid comprises a transgene encoding an antibody that binds to tau.

In some embodiments, the nucleic acid molecule comprising the transgene encoding a payload further comprises a nucleotide sequence encoding a linker (e.g., a linker connecting a heavy chain variable region (VH) and a light chain variable region (VL) of an antibody) and/or a cleavage site. In some embodiments, the nucleic acid molecule comprising the transgene encoding a payload further comprises a nucleotide sequence encoding a signal sequence.

In some embodiments, the nucleic acid encoding the payload may be constructed, e.g., organized, similar to, e.g., mirroring, the natural organization of an mRNA. In some embodiments, the nucleic acid encoding the payload may comprise coding and/or non-coding nucleotide sequences. In some embodiments, the nucleic acid encoding the payload may encode a coding and/or a non-coding RNA.

In such an embodiment, the nucleic acid comprising a transgene encoding a payload (e.g., an antibody or fragment thereof) is replicated and packaged into an AAV particle. In some embodiments, following transduction of a cell with an AAV particle comprising a payload (e.g., an antibody or fragment thereof), the cell expresses the payload. In some embodiments, the payload, e.g., antibody or fragment thereof, produced by a cell transduced by an AAV particle comprising the payload, is secreted from the cell.

In some embodiments, the encoded payload of a viral genome described herein comprises an anti-tau antibody or functional variant, e.g., fragment, thereof. In some embodiments, the functional variant is a humanized variant, such as a humanized variant comprising any one or more (e.g., all 6) CDR regions of any one of the antibodies in Table 4. In some embodiments, the encoded payload of a viral genome described herein comprises an anti-tau antibody or functional variant thereof described in WO 2021/211753, the contents of which are hereby incorporated by reference in its entirety.

In some embodiments, the nucleotide the encoded antibody comprises a heavy chain variable region (VH) and/or a light chain variable region (VL) sequences, e.g., as provided in Table 4, or variants or fragments thereof; optionally the polypeptide(s) further comprises a heavy chain constant region and/or a light chain constant region, such as those listed in Table 5. The encoded antibody may constitute a full-length antibody (e.g., comprising a VH and a heavy chain constant region, such as those listed in Table 5; and a VL and a light chain constant region, such as those listed in Table 5), or an antibody fragment thereof, such as Fab, F(ab′)₂, scFv, etc. The encoded antibody may also comprise a linker between the heavy and light chain sequences or the VH and VL sequences. In some embodiments, the encoded heavy chain or VH is located N-terminal relative to the encoded light chain or VL. In some embodiments, the encoded light chain or VL is located N-terminal relative to the encoded heavy chain or VH.

In some embodiments, the nucleotide sequence encoding the anti-tau antibody comprises, in the 5′ to 3′ direction, a nucleotide sequence encoding an light chain or a nucleotide sequence encoding a VL, a linker, and a nucleotide sequence encoding a heavy chain or a nucleotide sequence encoding a VH (e.g., light-linker-heavy or L.Linker.H or LH). In another embodiment, the nucleotide sequence encoding the payload does not comprise a linker.

In some embodiments, the nucleotide sequence encoding the anti-tau antibody comprises, in the 5′ to 3′ direction, a nucleotide sequence encoding an heavy chain or a nucleotide sequence encoding a VH, a linker, and a nucleotide sequence encoding a light chain or a nucleotide sequence encoding a VL (e.g., heavy-linker-light or H.Linker.L or HL). In another embodiment, the nucleotide sequence encoding the payload does not comprise a linker.

In some embodiments, the viral genome comprises a nucleotide sequence encoding a heavy chain. In some embodiments, the encoded heavy chain comprises an amino acid sequence or fragment thereof as provided in Table 4, and/or 5.

In some embodiments, the viral genome comprises a nucleotide sequence encoding a light chain. In some embodiments, the encoded light chain comprises an amino acid sequence or fragments thereof as provided in Table 4, and/or 5.

In some embodiments, the viral genome comprises a nucleotide sequence encoding an anti-tau antibody, wherein the encoded anti-tau antibody comprises at least one antigen-binding domain, e.g., a variable region or antigen binding fragment thereof, from an antibody described herein, e.g., from V0004, V0009, V0022, V0023, V0024, or V0052, e.g., as described in Tables 1 or 4, or a sequence substantially identical (e.g., having at least about 70%, 75%, 80%, 85%, 90%, 92%, 95%, 97%, 98%, or 99% sequence identity) to any of the aforesaid sequences. In some embodiments, the anti-tau antibody comprises at least one antigen-binding domain, e.g., a variable region or antigen binding fragment thereof, from an antibody described in WO 2021/211753, the contents of which are hereby incorporated by reference in its entirety, or a sequence substantially identical (e.g., having at least about 70%, 75%, 80%, 85%, 90%, 92%, 95%, 97%, 98%, or 99% sequence identity) to any of the antibody sequences disclosed in WO 2021/211753.

In some embodiments, the encoded anti-tau antibody comprises a heavy chain variable region from an antibody described herein, e.g., chosen from V0004, V0009, V0022, V0023, V0024, or V0052, e.g., as described in Table 4, or a sequence substantially identical (e.g., having at least about 70%, 75%, 80%, 85%, 90%, 92%, 95%, 97%, 98%, or 99% sequence identity) to any of the aforesaid sequences. In some embodiments, the heavy chain variable region comprises an amino acid sequence having at least one, two, or three modifications (e.g., substitutions, e.g., conservative substitutions), but not more than 30, 20, or 10 modifications (e.g., substitutions, e.g., conservative substitutions) of an amino acid sequence of a heavy chain variable region provided in Table 4.

In some embodiments, the nucleotide sequence encoding the anti-tau antibody comprises the nucleotide sequence of a heavy chain variable region from an antibody described herein, e.g., chosen from V0004, V0009, V0022, V0023, V0024, or V0052, e.g., as described in Table 3 or 4, or a nucleotide sequence substantially identical (e.g., having at least about 70%, 75%, 80%, 85%, 90%, 92%, 95%, 97%, 98%, or 99% sequence identity) to any of the aforesaid sequences.

In some embodiments, the encoded anti-tau antibody comprises a light chain variable region from an antibody described herein, e.g., chosen from V0004, V0009, V0022, V0023, V0024, or V0052, e.g., as described in Table 4, or a sequence substantially identical (e.g., having at least about 70%, 75%, 80%, 85%, 90%, 92%, 95%, 97%, 98%, or 99% sequence identity) to any of the aforesaid sequences. In some embodiments, the light chain variable region comprises an amino acid sequence having at least one, two, or three modifications (e.g., substitutions, e.g., conservative substitutions), but not more than 30, 20, or 10 modifications (e.g., substitutions, e.g., conservative substitutions) of an amino acid sequence of a light chain variable region provided in Table 4.

In some embodiments, the nucleotide sequence encoding the anti-tau antibody comprises the nucleotide sequence of a light chain variable region from an antibody described herein, e.g., chosen from V0004, V0009, V0022, V0023, V0024, or V0052, e.g., as described in Table 4, or a nucleotide sequence substantially identical (e.g., having at least about 70%, 75%, 80%, 85%, 90%, 92%, 95%, 97%, 98%, or 99% sequence identity) to any of the aforesaid sequences.

In some embodiments, the encoded anti-tau antibody comprises a heavy chain variable region and a light chain variable region from an antibody described herein, e.g., chosen from V0004, V0009, V0022, V0023, V0024, or V0052, e.g., as described in Table 4, or a sequence substantially identical (e.g., having at least about 70%, 75%, 80%, 85%, 90%, 92%, 95%, 97%, 98%, or 99% sequence identity) to any of the aforesaid sequences. In some embodiments, the anti-tau antibody comprises a heavy chain variable region comprising an amino acid sequence having at least one, two, or three modifications (e.g., substitutions, e.g., conservative substitutions), but not more than 30, 20, or 10 modifications (e.g., substitutions, e.g., conservative substitutions) of an amino acid sequence of a heavy chain variable region provided in Table 4; and a light chain variable region comprising an amino acid sequence having at least one, two, or three modifications (e.g., substitutions, e.g., conservative substitutions), but not more than 30, 20, or 10 modifications (e.g., substitutions, e.g., conservative substitutions) of an amino acid sequence of a light chain variable region provided in Table 4.

In some embodiments, the anti-tau antibody comprises a heavy chain constant region, e.g., a human IgG1, IgG2, IgG3, or IgG4 constant regions, or a murine IgG1, IgG2A, IgG2B, IgG2C, or IgG3 constant regions. In some embodiments, the heavy chain constant comprises an amino acid sequence set forth in Table 5, or a sequence substantially identical (e.g., having at least about 70%, 75%, 80%, 85%, 90%, 92%, 95%, 97%, 98%, or 99% sequence identity) to any of the aforesaid sequences. In some embodiments, a nucleic acid encoding the heavy chain constant region comprises a nucleotide sequence set forth in Table 5, or a nucleotide sequence substantially identical (e.g., having at least about 70%, 75%, 80%, 85%, 90%, 92%, 95%, 97%, 98%, or 99% sequence identity) to any of the aforesaid sequences.

In some embodiments, the encoded anti-tau antibody comprises a light chain constant region, e.g., a kappa light chain constant region, e.g., a human kappa or lambda light chain constant region or a murine kappa or lambda light chain constant region. In some embodiments, the light chain constant comprises an amino acid sequence set forth in Table 5, or a sequence substantially identical (e.g., having at least about 70%, 75%, 80%, 85%, 90%, 92%, 95%, 97%, 98%, or 99% sequence identity) to any of the aforesaid sequences. In some embodiments, nucleic acid encoding the light chain constant region comprises a nucleotide sequence set forth in Table 5, or a nucleotide sequence substantially identical (e.g., having at least about 70%, 75%, 80%, 85%, 90%, 92%, 95%, 97%, 98%, or 99% sequence identity) to any of the aforesaid sequences.

In some embodiments, the encoded anti-tau antibody comprises a heavy chain constant region and a light chain constant region. In some embodiments, the heavy chain constant region and the light chain constant region comprise an amino acid sequence set forth in Table 5, or a sequence substantially identical (e.g., having at least about 80%, 85%, 90%, 92%, 95%, 97%, 98%, or 99% sequence identity) thereto. In some embodiments, the nucleotide sequence encoding the anti-tau antibody comprises the nucleotide sequence of a heavy chain constant region and the nucleotide sequence of a kappa or lambda light chain constant region. In some embodiments, the nucleotide sequence encoding the heavy chain constant region and light chain constant region comprise a nucleotide sequence set forth in Table 5, or a nucleotide sequence substantially identical (e.g., having at least about 80%, 85%, 90%, 92%, 95%, 97%, 98%, or 99% sequence identity) thereto.

In some embodiments, the encoded anti-tau antibody comprises a heavy chain variable region and a constant region, a light chain variable region and a constant region, or both, comprising an amino acid sequence of Table 4 for variable region, and an amino acid sequence of Table 5 for constant region; or is encoded by a nucleic acid sequence of Table 4, and 5; or a sequence substantially identical (e.g., having at least about 80%, 85%, 90%, 92%, 95%, 97%, 98%, or 99% sequence identity) to any of the aforesaid sequences.

In some embodiments, the encoded anti-tau antibody comprises at least one, two, or three complementarity determining regions (CDRs) from a heavy chain variable region comprising an amino acid sequence in Table 4, or is encoded by a nucleic acid sequence in Table 4; or a sequence substantially identical (e.g., having at least about 70%, 75%, 80%, 85%, 90%, 92%, 95%, 97%, 98%, or 99% sequence identity) to any of the aforesaid sequences. In some embodiments, one, two, three, four, five, or all of the CDRs have one, two, three, four, five or more changes, e.g., amino acid substitutions, insertions, or deletions, relative to the amino acid sequence shown in Table 4, or encoded by a nucleotide sequence shown in Table 4. In some embodiments, the encoded anti-tau antibody includes a substitution in a heavy chain CDR, e.g., one or more substitutions in a CDR1, CDR2 and/or CDR3 of the heavy chain.

In some embodiments, the encoded anti-tau antibody comprises at least one, two, or three complementarity determining regions (CDRs) from a light chain variable region comprising an amino acid sequence in Table 4, or is encoded by a nucleic acid sequence in Table 4; or a sequence substantially identical (e.g., having at least about 80%, 85%, 90%, 92%, 95%, 97%, 98%, or 99% sequence identity) to any of the aforesaid sequences. In some embodiments, one, two, three, four, five, or all of the CDRs have one, two, three, four, five or more changes, e.g., amino acid substitutions, insertions, or deletions, relative to the amino acid sequence shown in Table 4, or encoded by a nucleotide sequence shown in Table 4. In some embodiments, the anti-tau antibody includes a substitution in a light chain CDR, e.g., one or more substitutions in a CDR1, CDR2 and/or CDR3 of the light chain.

In some embodiments, the encoded anti-tau antibody comprises at least one, two, three, four, five or six CDRs (or collectively all of the CDRs) from a heavy and light chain variable region comprising an amino acid sequence shown in Table 4, or is encoded by a nucleotide sequence shown in Table 4. In some embodiments, one or more of the CDRs (or collectively all of the CDRs) have one, two, three, four, five, six or more changes, e.g., amino acid substitutions, insertions, or deletions, relative to the CDRs shown in Table 4, or encoded by a nucleotide sequence shown in Table 4.

In some embodiments, the encoded anti-tau antibody comprises all three CDRs from a heavy chain variable region, all three CDRs from light chain variable region, or both (e.g., all six CDRs from a heavy chain variable region and a light chain variable region) comprising an amino acid sequence shown in Table 4, or is encoded by a nucleotide sequence shown in Table 4.

In some embodiments, an encoded anti-tau antibody of the present disclosure may include CDRs identified through CDR analysis of variable domain sequences presented herein via co-crystallography with bound antigen; by computational assessments based on comparisons with other antibodies (e.g., see Strohl, W. R. Therapeutic Antibody Engineering. Woodhead Publishing, Philadelphia PA. 2012. Ch. 3, p47-54); or Kabat, Chothia, Al-Lazikani, Lefranc, or Honegger numbering schemes, as described previously.

In some embodiments, the viral genome may comprise one or more components which have been codon-optimized. Codon-optimization may be achieved by any method known to one with skill in the art such as, but not limited to, by a method according to Genscript, EMBOSS, Bioinformatics, NUS, NUS2, Geneinfinity, IDT, NUS3, GregThatcher, Insilico, Molbio, N2P, Snapgene, and/or VectorNTI. Antibody heavy and/or light chain sequences within the same viral genome may be codon-optimized according to the same or according to different methods.

The viral genome may encode an antibody fragment, such as, but not limited to Fab, F(ab′)² or scFv fragments. In some embodiments, the viral genome encodes a Fab antibody fragment. In another embodiment, the viral genome encodes an F(ab′)₂ antibody fragment. In some embodiments, the viral genome encodes an scFv.

This disclosure also provides in some embodiments, nucleic acids, cells, AAV vectors, and AAV particles comprising the above viral genome.

### Payload Component: Signal Sequence Region

In some embodiments, the nucleotide sequence comprising the transgene encoding the payload, e.g., an antibody or fragment thereof, comprises a nucleotide sequence encoding a signal sequence (e.g., a signal sequence region herein). In some embodiments, the nucleotide sequence comprising the transgene encoding the payload comprises two signal sequence regions. In some embodiments, the nucleotide sequence comprising the transgene encoding the payload comprises three or more signal sequence regions.

In some embodiments, the nucleotide sequence encoding the signal sequence is located 5′ relative to the nucleotide sequence encoding the VH and/or the heavy chain. In some embodiments, the nucleotide sequence encoding the signal sequence is located 5′ relative to the nucleotide sequence encoding the VL and/or the light chain. In some embodiments, the encoded VH, VL, heavy chain, and/or light chain of the encoded antibody comprises a signal sequence at the N-terminus, wherein the signal sequence is optionally cleaved during cellular processing and/or localization of the antibody.

In some embodiments, the signal sequence comprises any one of the signal sequences provided in Table 14 or a functional variant thereof. In some embodiments, the encoded signal sequence comprises an amino acid sequence encoded by any one of the nucleotide sequences provided in Table 14, or an amino acid sequence with at least 70%, 75%, 80%, 85%, 90%, 95%, or 99% sequence identity thereto. In some embodiments, the nucleotide sequence encoding the signal sequence comprises any one of the nucleotide sequences provided in Table 14, or a nucleotide sequence with at least 70%, 75%, 80%, 85%, 90%, 95%, or 99% sequence identity thereto.

In some embodiments, the nucleotide sequence encoding the signal sequence comprises the nucleotide sequence of SEQ ID NO: 1083, or a nucleotide sequence with at least 70%, 75%, 80%, 85%, 90%, 95%, or 99% sequence identity thereto. In some embodiments, the nucleotide sequence is located 5′ relative to the nucleotide sequence encoding the VH and/or heavy chain of the antibody. In some embodiments, the nucleotide sequence encoding the signal sequence comprises the nucleotide sequence of SEQ ID NO: 1083 and is located 5′ relative to the nucleotide sequence encoding the VH and/or heavy chain of the antibody.

In some embodiments, the nucleotide sequence encoding the signal sequence comprises the nucleotide sequence of SEQ ID NO: 1085, or a nucleotide sequence with at least 70%, 75%, 80%, 85%, 90%, 95%, or 99% sequence identity thereto. In some embodiments, the nucleotide sequence is located 5′ relative to the nucleotide sequence encoding the VL and/or light chain of the antibody. In some embodiments, the nucleotide sequence encoding the signal sequence comprises the nucleotide sequence of SEQ ID NO: 1085 and is located 5′ relative to the nucleotide sequence encoding the VL and/or light chain of the antibody.

In some embodiments, the encoded signal sequence comprises the amino acid sequence of SEQ ID NO: 1; an amino acid sequence comprising one, two, three, but no more than four different amino acids relative to SEQ ID NO: 1; an amino acid sequence comprising one, two, three, but no more than four modifications, e.g., substitutions (e.g., conservative substitutions), relative to SEQ ID NO: 1; or an amino acid sequence with at least 70%, 75%, 80%, 85%, 90%, 95%, or 99% sequence identity thereto. In some embodiments, the encoded signal sequence is located N-terminal relative to the encoded VH and/or heavy chain of the encoded antibody. In some embodiments, the encoded signal sequence comprises the amino acid sequence of SEQ ID NO: 1 and is located N-terminal to the encoded VH and/or heavy chain of the encoded antibody.

In some embodiments, the encoded signal sequence comprises the amino acid sequence of SEQ ID NO: 2; an amino acid sequence comprising one, two, three, but no more than four different amino acids relative to SEQ ID NO: 2; an amino acid sequence comprising one, two, three, but no more than four modifications, e.g., substitutions (e.g., conservative substitutions), relative to SEQ ID NO: 2; or an amino acid sequence with at least 70%, 75%, 80%, 85%, 90%, 95%, or 99% sequence identity thereto. In some embodiments, the encoded signal sequence is located N-terminal relative to the encoded VL and/or light chain of the encoded antibody. In some embodiments, the encoded signal sequence comprises the amino acid sequence of SEQ ID NO: 2 and is located N-terminal to the encoded VL and/or light chain of the encoded antibody.

TABLE 14
Signal Sequence Regions

| Sequence | SEQ ID | |
|---|---|---|
| Region Name | NO | Sequence |
| Signal 1 | 1071 | ATGGCGACGGGTTCAAGAACTTCCCTACTTCTTGCATTTGGCCTGCTTTGTTTGCCG |
| (hGH-2 (84)) | | TGGTTACAGGAGGGCTCGGCAGCTGCC |
| Signal 2 | 1072 | GCCGCCACCATGGCGACGGGTTCAAGAACTTCCCTACTTCTTGCATTTGGCCTGCTT |
| (hGH-2 (93)) | | TGTTTGCCGTGGTTACAGGAGGGCTCGGCAGCTGCC |
| Signal 3 | 1073 | GCCGCCACCATGGGGGTGCACGAATGTCCTGCCTGGCTGTGGCTTCTCCTGTCCCTG |
| (EPOh) | | CTGTCGCTCCCTCTGGGCCTCCCAGTCCTGGGCGCTGCC |
| Signal 4 | 1074 | GCCGCCACCATGGGAGTCAAAGTTCTGTTTGCCCTGATCTGCATCGCTGTGGCCGAG |
| (Gaussia Luc | | GCCGCTGCC |
| 100) | | |
| Signal 5 | 1075 | GCCGCCACCATGAAACACCTGTGGTTCTTCCTCCTGCTGGTGGCAGCTCCCAGATGG |
| (H3) | | GTCCTGTCCGCTGCC |
| Signal 6 | 1076 | GCCGCCACCATGGACCTCCTGCACAAGAACATGAAACACCTGTGGTTCTTCCTCCTC |
| (H8) | | CTGGTGGCAGCTCCCAGATGGGTGCTGTCCGCTGCC |
| Signal 7 | 1077 | GCCGCCACCATGGCTTTCCTCTGGCTCCTCTCCTGCTGGGCCCTCCTGGGTACCACC |
| (hCtry59) | | TTCGGCGCTGCC |
| Signal 8 | 1078 | GCCGCCACCATGCCCCCCAAGAAGTGCCTGCTGCTGCTGCTGACCCTGCTGCTGCTG |
| (HMM24) | | ATCTCCACCACCTTCGGCGCTGCC |
| Signal 9 | 1079 | GCCGCCACCATG |
| (Kozak) | | |
| Signal 10 | 1080 | GCCGCCACCATGGACATGAGGGTCCCTGCTCAGCTCCTGGGGCTCCTGCTGCTCTGG |
| (L1) | | CTCTCAGGTGCCAGATGTGCTGCC |
| Signal 11 | 1081 | GCCGCCACCATGGAAATCAAGGTGCTGTTTGCCCTCATCTGTATTGCTGTTGCTGAG |
| (Lucia) | | GCAGCTGCC |
| Signal 12 | 1082 | GCCGCCACCATGGAGACAGACACACTCCTGCTATGGGTACTGCTGCTCTGGGTTCCA |
| (pSEc) | | GGTTCCACTGGTGACGCTGCC |
| Signal 13 | 1083 | ATGAACTTCGGGCTCAGCTTGATTTTCCTTGTCCTTGTTTTAAAAGGTGTCCAGTGT |
| DNA | | |
| (HC signal) | | |
| Signal 13 | 1 | MNFGLSLIFLVLVLKGVQC |
| Amino acid | | |
| (HC signal) | | |
| Signal 14 | 1084 | ATGGGATGGAGCTGGATCTTTCTCTTTCTCCTGTCAGGAACTACAGGTGTCCTCTCT |
| (HC signal) | | |
| Signal 15 | 1085 | ATGAAGTTGCCTGTTAGGCTGTTGGTGCTGATGTTCTGGATTCCTGCTTCCAGCAGT |
| DNA | | |
| (LC signal) | | |
| Signal 15 | 2 | MKLPVRLLVLMFWIPASSS |
| Amino Acid | | |
| (LC signal) | | |
| Signal 16 | 1086 | ATGAACTTCGGGCTCAGCTTGATTTTCCTTGTCCTTGTTTTAAAAGGTGTCCAGTGT |
| (HC signal) | | GAGGTGAAGCTGGTGGAGAGCGGCGGCGACCTGGTGAAGCCTGGCGGCAGCCTGAAG |
| | | CTGAGCTGCGCCGCCAGCGGCTTCACCTTCAGCAGCTACGCCATGAGCTGGGTGAGA |
| | | CAGAACCCTGAGAAGAGACTGGAGTGGGTGGCCAGCATCAGCAAGGGCGGCAACACC |
| | | TACTACCCTAACAGCGTGAAGGGCAGATTCACCATCAGCAGAGACAACGCCAGAAAC |
| | | ATCCTGTACCTGCAGATGAGCAGCCTGAGAAGCGAGGACACCGCCCTGTACTACTGC |
| | | GCCAGAGGCTGGGGCGACTACGGCTGGTTCGCCTACTGGGGCCAGGTGACCCTGGTG |
| | | ACCGTGAGCGCC |
| Signal 17 | 203 | MGWTLVFLFLLSVTAGVHS |
| (HC signal) | | |
| Signal 17 DNA | 204 | ATGGGCTGGACCCTGGTGTTTCTCTTCCTGCTGAGCGTGACCGCCGGCGTGCACAGC |
| (HC signal) | | |
| Signal 18 | 205 | MVSSAQFLGLLLLCFQGTRC |
| (LC signal) | | |
| Signal 18 DNA | 206 | ATGGTGAGCAGCGCTCAGTTCCTGGGCCTGCTGCTGCTGTGTTTTCAAGGCACAAGA |
| (LC signal) | | TGT |
| Signal 18 DNA | 207 | ATGGTGAGCAGCGCTCAGTTCCTGGGCCTGCTGCTGCTGTGCTTCCAAGGCACAAGA |
| (LC signal) | | TGC |
| Signal 18 DNA | 208 | ATGGTGAGCAGCGCTCAGTTCCTGGGCCTGCTGCTGCTGTGTTTCCAAGGGACAAGA |
| (LC signal) | | TGT |

### Payload Component: Linkers

In some embodiments, the nucleic acid encoding the payload, e.g., an antibody (e.g., an anti-tau antibody described herein), comprises a nucleotide sequence encoding a linker. In some embodiments, the nucleic acid encoding the payload encodes two or more linkers. In some embodiments, the encoded linker comprises a linker provided in Table 15. In some embodiments, the encoded linker comprises an amino acid sequence encoded by any one of the nucleotide sequences provided in Table 15, or an amino acid sequence with at least 70%, 75%, 80%, 85%, 90%, 95%, or 99% sequence identity thereto. In some embodiments, the nucleotide sequence encoding the linker comprises any one of the nucleotide sequences provided in Table 15, or a nucleotide sequence with at least 70%, 75%, 80%, 85%, 90%, 95%, or 99% sequence identity thereto.

In some embodiments, the nucleotide sequence encoding the linker sequence(s) comprises the nucleotide sequence of any one of SEQ ID NOs: 1724-1739, 2244-2259, 5161, 5162, 5243, 5347, or 5348, or a sequence having at least 70%, 75%, 80%, 85%, 90%, 95%, or 99% sequence identity thereto. In some embodiments, the encoded linker comprises the amino acid sequence of any one of SEQ ID NOs: 601-609; an amino acid sequence comprising one, two, three, but no more than four different amino acids relative to any one of SEQ ID NOs: 601-609; an amino acid sequence comprising one, two, three, but no more than four modifications, e.g., substitutions (e.g., conservative substitutions) relative to any one of SEQ ID NOs: 601-609; or an amino acid sequence having at least 70%, 75%, 80%, 85%, 90%, 95%, or 99% sequence identity to any one of SEQ ID NOs: 601-609.

TABLE 15
Linkers

| | SEQ | |
|---|---|---|
| | ID | |
| Description | NO | Sequence |
| Furin | 1724 | AGAAAGAGGCGA |
| Furin | 1725 | CGGGCCAAGCGG |
| T2A | 1726 | GAGGGCAGAGGAAGTCTTCTAACATGCGGTGACGTGGAGGAGAATCCCGGCCCT |
| F2A | 1727 | GGAAGCGGAGTGAAACAGACTTTGAATTTTGACCTTCTCAAGTTGGCGGGAGACGTG |
| | | GAGTCCAACCCTGGACCT |
| P2A | 1728 | GGAAGCGGAGCTACTAACTTCAGCCTGCTGAAGCAGGCTGGAGACGTGGAGGAGAAC |
| | | CCTGGACCT |
| SG4S (SEQ ID | 1729 | TCCGGAGGCGGCGGCAGC |
| NO: 3) | | |
| (G4S)3 (SEQ | 1730 | GGTGGTGGTGGTAGCGGCGGCGGCGGCTCTGGTGGTGGTGGATCC |
| ID NO: 5348) | | |
| DNA | | |
| (G4S)5 (SEQ | 1731 | GGCGGAGGTGGCTCCGGAGGCGGAGGCAGCGGCGGAGGTGGGTCTGGCGGAGGCGGG |
| ID NO: 604) | | TGCGGCGGAGGTGGCTCC |
| DNA | | |
| IRES | 1732 | AATTCCGCCCCTCTCCCCCCCCCCCCTCTCCCTCCCCCCCCCCTAACGTTACTGGCC |
| | | GAAGCCGCTTGGAATAAGGCCGGTGTGCGTTTGTCTATATGTTATTTTCCACCATAT |
| | | TGCCGTCTTTTGGCAATGTGAGGGCCCGGAAACCTGGCCCTGTCTTCTTGACGAGCA |
| | | TTCCTAGGGGTCTTTCCCCTCTCGCCAAAGGAATGCAAGGTCTGTTGAATGTCGTGA |
| | | AGGAAGCAGTTCCTCTGGAAGCTTCTTGAAGACAAACAACGTCTGTAGCGACCCTTT |
| | | GCAGGCAGCGGAACCCCCCACCTGGCGACAGGTGCCTCTGCGGCCAAAAGCCACGTG |
| | | TATAAGATACACCTGCAAAGGCGGCACAACCCCAGTGCCACGTTGTGAGTTGGATAG |
| | | TTGTGGAAAGAGTCAAATGGCTCTCCTCAAGCGTATTCAACAAGGGGCTGAAGGATG |
| | | CCCAGAAGGTACCCCATTGTATGGGATCTGATCTGGGGCCTCGGTGCACATGCTTTA |
| | | CATGTGTTTAGTCGAGGTTAAAAAAACGTCTAGGCCCCCCGAACCACGGGGACGTGG |
| | | TTTTCCTTTGAAAAACACGATGATAATATGGCCACAACC |
| IRES-2 | 1733 | TAACGAATTCCGCCCCTCTCCCCCCCCCCCCTCTCCCTCCCCCCCCCCTAACGTTAC |
| | | TGGCCGAAGCCGCTTGGAATAAGGCCGGTGTGCGTTTGTCTATATGTTATTTTCCAC |
| | | CATATTGCCGTCTTTTGGCAATGTGAGGGCCCGGAAACCTGGCCCTGTCTTCTTGAC |
| | | GAGCATTCCTAGGGGTCTTTCCCCTCTCGCCAAAGGAATGCAAGGTCTGTTGAATGT |
| | | CGTGAAGGAAGCAGTTCCTCTGGAAGCTTCTTGAAGACAAACAACGTCTGTAGCGAC |
| | | CCTTTGCAGGCAGCGGAACCCCCCACCTGGCGACAGGTGCCTCTGCGGCCAAAAGCC |
| | | ACGTGTATAAGATACACCTGCAAAGGCGGCACAACCCCAGTGCCACGTTGTGAGTTG |
| | | GATAGTTGTGGAAAGAGTCAAATGGCTCTCCTCAAGCGTATTCAACAAGGGGCTGAA |
| | | GGATGCCCAGAAGGTACCCCATTGTATGGGATCTGATCTGGGGCCTCGGTGCACATG |
| | | CTTTACATGTGTTTAGTCGAGGTTAAAAAAACGTCTAGGCCCCCCGAACCACGGGGA |
| | | CGTGGTTTTCCTTTGAAAAACACGATGATAATATGGCCACAACCGCCGCCACC |
| hIgG2 hinge | 1734 | GTGGAGAGGAAGTGCTGCGTGGAGTGCCCACCATGCCCTGCCCCTCCTGTGGCC |
| hIgG3 hinge | 1735 | GAGCTCAAAACCCCACTTGGTGACACAACTCACACATGCCCACGGTGCCCAGAGCCC |
| | | AAATCTTGTGACACACCTCCCCCGTGCCCACGGTGCCCAGCACCTGAACTC |
| hIgG3-2 hinge | 1736 | GAGCTCAAAACCCCACTTGGTGACACAACTCACACATGCCCACGGTGCCCAGAGCCC |
| | | AAATCTTGTGACACACCTCCCCCGTGCCCACGGTGCCCAGAGCCCAAATCTTGTGAC |
| | | ACACCTCCCCCATGCCCACGGTGCCCAGCACCTGAACTC |
| hIgG3-3 hinge | 1737 | GAGCTCAAAACCCCACTTGGTGACACAACTCACACATGCCCACGGTGCCCAGAGCCC |
| | | AAATCTTGTGACACACCTCCCCCGTGCCCACGGTGCCCAGAGCCCAAATCTTGTGAC |
| | | ACACCTCCCCCATGCCCACGGTGCCCAGAGCCCAAATCTTGTGACACACCTCCCCCG |
| | | TGCCCAAGGTGCCCAGCACCTGAACTC |
| msiGG-1 hinge | 1738 | GTGCCCAGGGATTGTGGTTGTAAGCCTTGCATATGTACAGTCCCA |
| msiGG1 hinge | 1739 | GTGCCCAGGGATTGTGGT |
| HigG3 hinge | 2244 | GAGCTCAAAACCCCACTTGGTGACACAACTCACACATGCCCACGGTGCCCAGAGCCC |
| | | AAATCTTGTGACACACCTCCCCCGTGCCCACGGTGCCCAGAGCCCAAATCTTGTGAC |
| | | ACAACTCACACATGCCCACGGTGCCCAGAGCCCAAATCTTGTGACACACCTCCCCCG |
| | | TGCCCAAGGTGCCCAGCACCTGAACTC |
| G4S (SEQ ID | 2245 | GGTGGTGGTGGATCC |
| NO: 601) | | |
| DNA | | |
| G4S | 601 | GGGGS |
| Amino Acid | | |
| (G4S)2 (SEQ | 2246 | GGTGGTGGTGGATCCGGTGGTGGTGGATCC |
| ID NO: 602) | | |
| DNA | | |
| (G4S)2 | 602 | GGGGSGGGGS |
| Amino Acid | | |
| (G4S)3 (SEQ | 2247 | GGTGGTGGTGGATCCGGTGGTGGTGGATCCGGTGGTGGTGGATCC |
| ID NO: 5348) | | |
| DNA | | |
| (G4S)4 (SEQ | 2248 | GGTGGTGGTGGATCCGGTGGTGGTGGATCCGGTGGTGGTGGATCCGGTGGTGGTGGA |
| ID NO: 603) | | TCC |
| DNA | | |
| (G4S)4 | 603 | GGGGSGGGGSGGGGSGGGGS |
| Amino Acid | | |
| (G4S)5 (SEQ | 2249 | GGTGGTGGTGGTAGCGGCGGCGGCGGCTCTGGTGGTGGTGGATCCGGTGGTGGTGGA |
| ID NO: 604) | | TCCGGTGGTGGTGGATCC |
| DNA | | |
| (G4S)5 | 604 | GGGGSGGGGSGGGGSGGGGSGGGGS |
| Amino Acid | | |
| (G4S)5 (SEQ | 2250 | GGTGGTGGTGGATCCGGTGGTGGTGGATCCGGTGGTGGTGGATCCGGTGGTGGTGGA |
| ID NO: 604) | | TCCGGTGGTGGTGGATCC |
| DNA | | |
| (G4S)6 (SEQ | 2251 | GGTGGTGGTGGTAGCGGCGGCGGCGGCTCTGGTGGTGGTGGATCCGGTGGTGGTGGA |
| ID NO: 605) | | TCCGGTGGTGGTGGATCCGGTGGTGGTGGATCC |
| DNA | | |
| (G4S)6 | 605 | GGGGSGGGGSGGGGSGGGGSGGGGSGGGGS |
| Amino Acid | | |
| (G4S)8 (SEQ | 2252 | GGTGGTGGTGGTAGCGGCGGCGGCGGCTCTGGTGGTGGTGGATCCGGTGGTGGTGGA |
| ID NO: 607) | | TCCGGTGGTGGTGGATCCGGTGGTGGTGGATCCGGTGGTGGTGGATCCGGTGGTGGT |
| DNA | | GGATCC |
| (G4S)8 (SEQ | 2253 | GGTGGTGGTGGATCCGGTGGTGGTGGATCCGGTGGTGGTGGATCCGGTGGTGGTGGA |
| ID NO: 607) | | TCCGGTGGTGGTGGATCCGGTGGTGGTGGATCCGGTGGTGGTGGATCCGGTGGTGGT |
| DNA | | GGATCC |
| (G4S)8 | 607 | GGGGSGGGGSGGGGSGGGGSGGGGSGGGGSGGGGSGGGGS |
| Amino Acid | | |
| (G4S)4 (SEQ | 2254 | GGTGGTGGTGGTAGCGGCGGCGGCGGCTCTGGTGGTGGTGGATCCGGTGGTGGTGGA |
| ID NO: 603) | | TCC |
| DNA | | |
| (G4S)6 (SEQ | 2259 | GGTGGTGGTGGATCCGGTGGTGGTGGATCCGGTGGTGGTGGATCCGGTGGTGGTGGA |
| ID NO: 605) | | TCCGGTGGTGGTGGATCCGGTGGTGGTGGATCC |
| DNA | | |
| G4S (SEQ ID | 5161 | GGAGGAGGAGGAAGT |
| NO: 601) | | |
| DNA | | |
| G4S (SEQ ID | 5162 | GGAGGTGGAGGTTCT |
| NO: 601) | | |
| DNA | | |
| G4S | 608 | GGGGS |
| Amino Acid | | |
| GS | 5243 | GGCTCT |
| DNA | | |
| GS | 609 | GS |
| Amino Acid | | |
| (G4S)3 (SEQ | 5347 | GGTGGTGGTGGATCCGGAGGAGGAGGAAGTGGAGGTGGAGGTTCT |
| ID NO: 5348) | | |
| DNA | | |
| (G4S)3 | 5348 | GGGGSGGGGSGGGGS |
| Amino acid | | |

In some embodiments, any of the antibodies described herein can have a linker, e.g., a flexible polypeptide linker, of varying lengths, connecting the variable domains (e.g., the VH and the VL) of the antigen binding domain of the antibody. For example, a (Gly4-Ser)n linker, wherein n is 0, 1, 2, 3, 4, 5, 6, 7, or 8 (SEQ ID NO: 802) can be used (e.g., any one of SEQ ID NOs: 1730-1731, 2245-2254, 2259, 5161-5162, 5347, or 5348). In some embodiments, the antibody binds to tau.

In some embodiments, the encoded linker comprises an enzymatic cleavage site, e.g., for intracellular and/or extracellular cleavage. In some embodiments, the linker is cleaved to separate the VH and the VL of the antigen binding domain and/or the heavy chain and light chain of the antibody (e.g., an anti-tau antibody described herein). In some embodiments, the encoded linker comprises a furin linker or afunctional variant. In some embodiments, the nucleotide sequence encoding the furin linker comprises the nucleotide sequence of SEQ ID NO: 1724, or a nucleotide sequence with at least 70%, 75%, 80%, 85%, 90%, 95%, or 99% sequence identity thereto. In some embodiments, furin cleaves proteins downstream of abasic amino acid target sequence (e.g., Arg-X-(Arg/Lys)-Arg) (e.g., as described in Thomas, G., 2002. Nature Reviews Molecular Cell Biology 3(10): 753-66; the contents of which are herein incorporated by reference in its entirety). In some embodiments, the encoded linker comprises a 2A self-cleaving peptide (e.g., a 2A peptide derived from foot-and-mouth disease virus (H2A), porcine teschovirus-1 (P2A), Thoseaasigna virus (n2A), or equine rhinitis A virus (E2A)).

In some embodiments, the encoded linker comprises a T2A self-cleaving peptide linker. In some embodiments, the nucleotide sequence encoding the n2A linker comprises the nucleotide sequence of SEQ ID NO: 1726, or a nucleotide sequence with at least 70%, 75%, 80%, 85%, 90%, 95%, or 99% sequence identity thereto. In some embodiments, the nucleic acid encoding the payload encodes a furin linker and a T2A linker.

### Exemplary Viral Genome Encoding an Anti-Tau Antibody

In some embodiments, a viral genome, e.g., an AAV viral genome, described herein, comprises a promoter operably linked to a transgene encoding an anti-tau antibody. In some embodiments, the viral genome further comprises an inverted terminal repeat region, an enhancer, an intron, a polyA region, or a combination thereof. In some embodiments, the AAV viral genome further comprises a miR binding site.

In some embodiments, the viral genome comprises an inverted terminal repeat sequence region (ITR) provided in Table 10, or a nucleotide sequence with at least 70%, 75%, 80%, 85%, 90%, 95% or 99% sequence identity to any of the ITR sequences in Table 10.

This disclosure also provides in some embodiments, an antibody encoded by any of the nucleotide sequences provided in Table 4 and 5 or a nucleotide sequence having at least about 70%, 75%, 80%, 85%, 90%, 92%, 95%, 97%, 98%, or 99% sequence identity) to any of the aforesaid sequences. In some embodiments, the encoded antibody comprises an amino acid sequence provided in Table 4 and 5 or a nucleotide sequence having at least about 70%, 75%, 80%, 85%, 90%, 92%, 95%, 97%, 98%, or 99% sequence identity) to any of the aforesaid sequences.

In some embodiments, the viral genome of an AAV particle described herein comprises the nucleotide sequence, e.g., the nucleic acid sequence from the 5′ ITR to the 3′ ITR, of any of the nucleotide sequences in Tables 18 and 19, or a nucleotide sequence substantially identical (e.g., having at least about 70%, 75%, 80%, 85%, 90%, 92%, 95%, 97%, 98%, or 99% sequence identity) to any of the aforesaid sequences.

TABLE 18
Exemplary ITR-ITR sequences

| SEQ | |
|---|---|
| ID | |
| NO: | Sequence |
| 15 | ctgcgcgctcgctcgctcactgaggccgcccgggcaaagcccgggcgtcgggcgacctttggtcgcccg |
| | gcctcagtgagcgagcgagcgcgcagagagggagtggccaactccatcactaggggttccttgtagtta |
| | atgattaacccgccatgctacttatctaccagggtaatggggatcctctagaactatagctagtcGACA |
| | TTGATTATTGACTAGTTATTAATAGTAATCAATTACGGGGTCATTAGTTCATAGCCCATATATGGAGTT |
| | CCGCGTTACATAACTTACGGTAAATGGCCCGCCTGGCTGACCGCCCAACGACCCCCGCCCATTGACGTC |
| | AATAATGACGTATGTTCCCATAGTAACGCCAATAGGGACTTTCCATTGACGTCAATGGGTGGAGTATTT |
| | ACGGTAAACTGCCCACTTGGCAGTACATCAAGTGTATCATATGCCAAGTACGCCCCCTATTGACGTCAA |
| | TGACGGTAAATGGCCCGCCTGGCATTATGCCCAGTACATGACCTTATGGGACTTTCCTACTTGGCAGTA |
| | CATCTACGTATTAGTCATCGCTATTACCATGtcgaggccacgttctgcttcactctccccatctccccc |
| | ccctccccacccccaattttgtatttatttattttttaattattttgtgcagcgatgggggcggggggg |
| | gggggcgcgcgccaggcggggcggggcggggcgaggggcggggcggggcgaggcggagaggtgcggcgg |
| | cagccaatcagagcggcgcgctccgaaagtttccttttatggcgaggcggcggcggcggcggccctata |
| | aaaagcgaagcgcgcggcgggcgggagcaagcttcgtttagtgaaccgtcagatcgcctggagacgcca |
| | tccacgctgttttgacctccatagaagacaccgggaccgatccagcctccgcggattcgaatcccggcc |
| | gggaacggtgcattggaacgcggattccccgtgccaagagtgacgtaagtaccgcctatagagtctata |
| | ggcccacaaaaaatgctttcttcttttaatatacttttttgtttatcttatttctaatactttccctaa |
| | tctctttctttcagggcaataatgatacaatgtatcatgcctctttgcaccattctaaagaataacagt |
| | gataatttctgggttaaggcaatagcaatatttctgcatataaatatttctgcatataaattgtaactg |
| | atgtaagaggtttcatattgctaatagcagctacaatccagctaccattctgcttttattttatggttg |
| | ggataaggctggattattctgagtccaagctaggcccttttgctaatcatgttcatacctcttatcttc |
| | ctcccacagctcctgggcaacgtgctggtctgtgtgctggcccatcactttggcaaagaattgggattc |
| | gaaccggtgccgccaccatgaacttcgggctcagcttgattttccttgtccttgttttaaaaggtgtcc |
| | agtgtcaagtgcagctgcagcagcctggcaccgagctggtgaaacctggatctagcgtgaatctgagct |
| | gcaaggccagcggctttaccttcaccagatactggatgcactgggtcaaggaacggccaggccacggcc |
| | tggaatggatcggcaatatcaaccccaacaacggcggaacagatttcaacgagaagttcaagaacaagg |
| | ctacactgaccgtgcacaaaagctccaccaccgtgttcatccagctgagctctctgacaagcgaggaca |
| | gcgccgtgtactattgtgccagaggcaccggcaccggcgccatggactactggggccagggaacatctg |
| | tgacagtgtccagcgccaaaacgacacccccatctgtctatccactggcccctggatctgctgcccaaa |
| | ctaactccatggtgaccctgggatgcctggtcaagggctatttccctgagccagtgacagtgacctgga |
| | actctggatccctgtccagcggtgtgcacaccttcccagctgtcctgcagtctgacctctacactctga |
| | gcagctcagtgactgtcccctccagcacctggcccagcgagaccgtcacctgcaacgttgcccacccgg |
| | ccagcagcaccaaggtggacaagaaaattgtgcccagggattgtggttgtaagccttgcatatgtacag |
| | tcccagaagtatcatctgtcttcatcttccccccaaagcccaaggatgtgctcaccattactctgactc |
| | ctaaggtcacgtgtgttgtggtagacatcagcaaggatgatcccgaggtccagttcagctggtttgtag |
| | atgatgtggaggtgcacacagctcagacgcaaccccgggaggagcagttcaacagcactttccgctcag |
| | tcagtgaacttcccatcatgcaccaggactggctcaatggcaaggagttcaaatgcagggtcaacagtg |
| | cagctttccctgcccccatcgagaaaaccatctccaaaaccaaaggcagaccgaaggctccacaggtgt |
| | acaccattccacctcccaaggagcagatggccaaggataaagtcagtctgacctgcatgataacagact |
| | tcttccctgaagacattactgtggagtggcagtggaatgggcagccagcggagaactacaagaacactc |
| | agcccatcatggacacagatggctcttacttcgtctacagcaagctcaatgtgcagaagagcaactggg |
| | aggcaggaaatactttcacctgctctgtgttacatgagggcctgcacaaccaccatactgagaagagcc |
| | tctcccactctcctggtAgaaagaggcgagagggcagaggaagtcttctaacatgcggtgacgtggagg |
| | agaatcccggccctatgaagttgcctgttaggctgttggtgctgatgttctggattcctgcttccagca |
| | gtgatgtggtgatgacacagacccctctgagcctgcctgtgtccctcggcgaccaggccagcatcagct |
| | gtagaagcagccaatctctggtgcacaacaatggcatcacctacctgtactggtatctgcagaaacctg |
| | gccagagccccaagctgctgatctaccgggtgtccaatcggttcagcggagtgccagatagatttggcg |
| | gatctggcagcggcaccgacttcaccctgaagatctctagagtcgaggccgaggacctgggcgtgtact |
| | tctgcttccagggcacacacgtgcccagaaccttcggcggcggaacaaagctggaaatcaagcgggctg |
| | atgctgcaccaactgtatccatcttcccaccatccagtgagcagttaacatctggaggtgcctcagtcg |
| | tgtgcttcttgaacaacttctaccccaaagacatcaatgtcaagtggaagattgatggcagtgaacgac |
| | aaaatggcgtcctgaacagttggactgatcaggacagcaaagacagcacctacagcatgagcagcaccc |
| | tcacgttgaccaaggacgagtatgaacgacataacagctatacctgtgaggccactcacaagacatcaa |
| | cttcacccattgtcaagagcttcaacaggaatgagtgttaactcgaggacggggtgaactacgcctgag |
| | gatccgatctttttccctctgccaaaaattatggggacatcatgaagccccttgagcatctgacttctg |
| | gctaataaaggaaatttattttcattgcaatagtgtgttggaattttttgtgtctctcactcggcctag |
| | gtagataagtagcatggcgggttaatcattaactacaaggaacccctagtgatggagttggccactccc |
| | tctctgcgcgctcgctcgctcactgaggccgggcgaccaaaggtcgcccgacgcccgggctttgcccgg |
| | gcggcctcagtgagcgagcgagcgcgcag |

TABLE 19
Exemplary Viral Genome (ITR to ITR) sequences

| SEQ ID | |
|---|---|
| NO: | Description (5′ to 3′) |
| 15 | ITR (SEQ ID NO: 1035); CMVie enhance (SEQ ID NO: 1050); CBA promoter (SEQ ID |
| | NO: 1042); intron (SEQ ID NO: 1067); nucleotide sequence encoding a signal sequence |
| | (SEQ ID NO: 1083); nucleotide sequence encoding VH (SEQ ID NO: 7); a nucleotide |
| | sequence encoding an IgG1 heavy chain constant region (SEQ ID NO: 805); a furin |
| | cleavage site (SEQ ID NO: 1724); a T2A linker (SEQ ID NO: 1726); a nucleotide sequence |
| | encoding a signal sequence (SEQ ID NO: 1085); a nucleotide sequence encoding a VL |
| | (SEQ ID NO: 11); a nucleotide sequence encoding a IgG kappa light chain constant region |
| | (SEQ ID NO: 17); polyA sequence (SEQ ID NO: 1134); ITR (SEQ ID NO: 1037) |

In some embodiments, the viral genome of an AAV particle described herein comprises a nucleotide sequence comprising the all of the components or a combination of the components as described, e.g., in Tables 18 and 19, or a sequence having at least 70%, 75%, 80%, 85%, 90%, 95%, or 99% sequence identity to any of the aforesaid sequences.

In some embodiments, the present disclosure provides an anti-tau antibody encoded by the nucleotide sequence of SEQ ID NO: 15, or a nucleotide sequence having at least 70%, 75%, 80%, 85%, 90%, 95%, or 99% sequence identity thereto.

In some embodiments, a viral genome, e.g., a viral genome encoding an anti-tau antibody described herein, comprises the nucleotide sequence of SEQ ID NO: 15, or a nucleotide sequence having at least 70%, 75%, 80%, 85%, 90%, 95%, or 99% sequence identity thereto.

### AAV Production

The present disclosure provides methods for the generation of parvoviral particles, e.g. AAV particles, by viral genome replication in a viral replication cell.

In accordance with the disclosure, the viral genome comprising a payload region encoding an antibody, an antibody-based composition or fragment thereof, will be incorporated into the AAV particle produced in the viral replication cell. Methods of making AAV particles are well known in the art and are described in e.g., U.S. Pat. Nos. 6,204,059, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508, 5,064,764, 6,194,191, 6,566,118, 8,137,948; or International Publication Nos. WO1996039530, WO1998010088, WO1999014354, WO1999015685, WO1999047691, WO2000055342, WO2000075353, and WO2001023597; Methods In Molecular Biology, ed. Richard, Humana Press, NJ (1995); O'Reilly et al., Baculovirus Expression Vectors, A Laboratory Manual, Oxford Univ. Press (1994); Samulski et al., J. Vir. 63:3822-8 (1989); Kajigaya et al., Proc. Nat'l. Acad. Sci. USA 88: 4646-50 (1991); Ruffing et al., J. Vir. 66:6922-30 (1992); Kimbauer et al., Vir., 219:37-44 (1996); Zhao et al., Vir.272:382-93 (2000); the contents of each of which are herein incorporated by reference in their entirety. In some embodiments, the AAV particles are made using the methods described in WO2015191508, the contents of which are herein incorporated by reference in their entirety.

Viral replication cells commonly used for production of recombinant AAV viral vectors include but are not limited to 293 cells, COS cells, HeLa cells, KB cells, and other mammalian cell lines as described in U.S. Pat. Nos. 6,156,303, 5,387,484, 5,741,683, 5,691,176, and 5,688,676; U.S. patent publication No. 2002/0081721, and International Patent Publication Nos. WO 00/47757, WO 00/24916, and WO 96/17947, the contents of each of which are herein incorporated by reference in their entireties.

In some embodiments, the AAV particles of the present disclosure may be produced in insect cells (e.g., Sf9 cells).

In some embodiments, the AAV particles of the present disclosure may be produced using triple transfection.

In some embodiments, the AAV particles of the present disclosure may be produced in mammalian cells.

In some embodiments, the AAV particles of the present disclosure may be produced by triple transfection in mammalian cells.

In some embodiments, the AAV particles of the present disclosure may be produced by triple transfection in HEK293 cells.

The present disclosure provides a method for producing an AAV particle comprising the steps of: 1) co-transfecting competent bacterial cells with a bacmid vector and either a viral construct vector and/or AAV payload construct vector, 2) isolating the resultant viral construct expression vector and AAV payload construct expression vector and separately transfecting viral replication cells, 3) isolating and purifying resultant payload and viral construct particles comprising viral construct expression vector or AAV payload construct expression vector, 4) co-infecting a viral replication cell with both the AAV payload and viral construct particles comprising viral construct expression vector or AAV payload construct expression vector, 5) harvesting and purifying the viral particle comprising a parvoviral genome.

In some embodiments, the present disclosure provides a method for producing an AAV particle comprising the steps of 1) simultaneously co-transfecting mammalian cells, such as, but not limited to HEK293 cells, with a payload region, a construct expressing rep and cap genes and a helper construct, 2) harvesting and purifying the AAV particle comprising a viral genome.

In some embodiments, the viral construct vector(s) used for AAV production may contain a nucleotide sequence encoding the AAV capsid proteins where the initiation codon of the AAV VP1 capsid protein is a non-ATG, i.e., a suboptimal initiation codon, allowing the expression of a modified ratio of the viral capsid proteins in the production system, to provide improved infectivity of the host cell. In a non-limiting example, a viral construct vector may contain a nucleic acid construct comprising a nucleotide sequence encoding AAV VP1, VP2, and VP3 capsid proteins, wherein the initiation codon for translation of the AAV VP1 capsid protein is CTG, TTG, or GTG, as described in U.S. Pat. No. 8,163,543, the contents of which are herein incorporated by reference in its entirety.

In some embodiments, the viral construct vector(s) used for AAV production may contain a nucleotide sequence encoding the AAV rep proteins where the initiation codon of the AAV rep protein or proteins is a non-ATG. In some embodiments, a single coding sequence is used for the Rep78 and Rep52 proteins, wherein initiation codon for translation of the Rep78 protein is a suboptimal initiation codon, selected from the group consisting of ACG, TTG, CTG and GTG, that effects partial exon skipping upon expression in insect cells, as described in U.S. Pat. No. 8,512,981, the contents of which is herein incorporated by reference in its entirety, for example to promote less abundant expression of Rep78 as compared to Rep52, which may be advantageous in that it promotes high vector yields.

In some embodiments, the viral genome of the AAV particle optionally encodes a selectable marker. The selectable marker may comprise a cell-surface marker, such as any protein expressed on the surface of the cell including, but not limited to receptors, CD markers, lectins, integrins, or truncated versions thereof.

In some embodiments, selectable marker reporter genes are selected from those described in International Application No. WO 96/23810; Heim et al., Current Biology 2:178-182 (1996); Heim et al., Proc. Natd. Acad. Sci. USA (1995); or Heim et al., Science 373:663-664 (1995); WO 96/30540, the contents of each of which are incorporated herein by reference in their entireties).

The AAV viral genomes encoding an anti-tau antibody payload described herein may be useful in the fields of human disease, veterinary applications and a variety of in vivo and in vitro settings. The AAV particles of the present disclosure may be useful in the field of medicine for the treatment, prophylaxis, palliation or amelioration of neurological diseases and/or disorders. In some embodiments, the AAV particles are used for the prevention and/or treatment of a tauopathy.

Various embodiments herein provide a pharmaceutical composition comprising the AAV particles described herein and a pharmaceutically acceptable excipient.

Various embodiments herein provide a method of treating a subject in need thereof comprising administering to the subject a therapeutically effective amount of the pharmaceutical composition described herein.

Certain embodiments of the method provide that the subject is treated by a route of administration of the pharmaceutical composition selected from the group consisting of intravenous, intracerebroventricular, intraparenchymal, intrathecal, subpial and intramuscular, or a combination thereof. Certain embodiments of the method provide that the subject is treated for a tauopathy and/or other neurological disorder. In one aspect of the method, a pathological feature of the tauopathy or other neurological disorder is alleviated and/or the progression of the tauopathy or other neurological disorder is halted, slowed, ameliorated or reversed.

Various embodiments herein describe a method of decreasing the level of soluble tau in the central nervous system of a subject in need thereof comprising administering to said subject an effective amount of the pharmaceutical composition described herein.

Also described herein are compositions, methods, processes, kits and devices for the design, preparation, manufacture and/or formulation of AAV particles. In some embodiments, payloads, such as but not limited to anti-tau antibodies, may be encoded by payload constructs or contained within plasmids or vectors or recombinant adeno-associated viruses (AAVs).

The present disclosure also provides administration and/or delivery methods for vectors and viral particles, e.g., AAV particles, for the treatment or amelioration of neurological disease, such as, but not limited to tauopathy.

### III. Formulation and Delivery

### Pharmaceutical Compositions

Compounds and AAV particles disclosed herein may be prepared as pharmaceutical compositions. As used herein the term “pharmaceutical composition” refers to compositions including at least one active ingredient and, most often, a pharmaceutically acceptable excipient.

Relative amounts of the active ingredient (e.g. an antibody), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may include between 0.1% and 99% (w/w) of the active ingredient. By way of example, the composition may include between 0.1% and 100/6, e.g., between 0.5 and 50%, between 1-30%, between 5-80%, at least 80% (w/w) active ingredient.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, rats, birds, including commercially relevant birds such as poultry, chickens, ducks, geese, and/or turkeys.

In some embodiments, compositions are administered to humans, human patients, or subjects.

### Formulations

Compounds and AAV particles of the present disclosure can be formulated using one or more excipients to: (1) increase stability; (2) increase cell permeability; (3) permit the sustained or delayed release (e.g., from a sustained release formulation); and/or (4) alter the biodistribution (e.g., target an antibody to specific tissues or cell types). In addition to traditional excipients such as any and all solvents, dispersion media, diluents, or other liquid vehicles, dispersion or suspension aids, surface active agents, isotonic agents, thickening or emulsifying agents, preservatives, formulations of the present disclosure can include, without limitation, liposomes, lipid nanoparticles, polymers, lipoplexes, core-shell nanoparticles, peptides, proteins, transfected cells (e.g., for transplantation into a subject) and combinations thereof.

Pharmaceutical compositions described herein may be prepared by methods known or hereafter developed in the art of pharmacology. Such preparatory methods may include the step of associating the active ingredient with an excipient and/or one or more other accessory ingredients.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition including a predetermined amount of the active ingredient. The amount of the active ingredient is generally equal to the dosage of the active ingredient which would be administered to a subject and/or a convenient fraction of such a dosage such as, for example, one-half or one-third of such a dosage.

In some embodiments, the AAV particles may be formulated in phosphate buffered saline (PBS), in combination with an ethylene oxide/propylene oxide copolymer (also known as Pluronic or poloxamer).

In some embodiments, the AAV particles may be formulated in PBS with 0.001% Pluronic acid (F-68) (poloxamer 188) at a pH of about 7.0.

In some embodiments, the AAV particles may be formulated in PBS with 0.001% Pluronic acid (F-68) (poloxamer 188) at a pH of about 7.3.

In some embodiments, the AAV particles may be formulated in PBS with 0.001% Pluronic acid (F-68) (poloxamer 188) at a pH of about 7.4.

In some embodiments, the AAV particles may be formulated in a solution comprising sodium chloride, sodium phosphate and an ethylene oxide/propylene oxide copolymer.

In some embodiments, the AAV particles may be formulated in a solution comprising sodium chloride, sodium phosphate dibasic, sodium phosphate monobasic and poloxamer 188/Pluronic acid (F-68).

In some embodiments, the AAV particles may be formulated in a solution comprising about 180 mM sodium chloride, about 10 mM sodium phosphate and about 0.001% poloxamer 188. In some embodiments, this formulation may be at a pH of about 7.3. The concentration of sodium chloride in the final solution may be 150 mM-200 mM. As non-limiting examples, the concentration of sodium chloride in the final solution may be 150 mM, 160 mM, 170 mM, 180 mM, 190 mM or 200 mM. The concentration of sodium phosphate in the final solution may be 1 mM-50 mM. As non-limiting examples, the concentration of sodium phosphate in the final solution may be 1 mM, 2 mM, 3 mM, 4 mM, 5 mM, 6 mM, 7 mM, 8 mM, 9 mM, 10 mM, 15 mM, 20 mM, 25 mM, 30 mM, 40 mM, or 50 mM. The concentration of poloxamer 188 (Pluronic acid (F-68)) may be 0.0001%-1%. As non-limiting examples, the concentration of poloxamer 188 (Pluronic acid (F-68)) may be 0.0001%, 0.0005%, 0.001%, 0.005%, 0.01%, 0.05%, 0.1%, 0.5%, or 1%. The final solution may have a pH of 6.8-7.7. Non-limiting examples for the pH of the final solution include a pH of 6.8, 6.9, 7.0, 7.1, 7.2, 7.3, 7.4, 7.5, 7.6, or 7.7.

In some embodiments, the AAV particles of the invention may be formulated in a solution comprising about 1.05% sodium chloride, about 0.212% sodium phosphate dibasic, heptahydrate, about 0.025% sodium phosphate monobasic, monohydrate, and 0.001% poloxamer 188, at a pH of about 7.4. As a non-limiting example, the concentration of AAV particle in this formulated solution may be about 0.001%. The concentration of sodium chloride in the final solution may be 0.1-2.0%, with non-limiting examples of 0.1%, 0.25%, 0.5%, 0.75%, 0.95%, 0.96%, 0.97%, 0.98%, 0.99%, 1.00%, 1.01%, 1.02%, 1.03%, 1.04%, 1.05%, 1.06%, 1.07%, 1.08%, 1.09%, 1.10%, 1.25%, 1.50%, 1.75%, or 2%. The concentration of sodium phosphate dibasic in the final solution may be 0.100-0.300% with non-limiting examples including 0.100%, 0.125%, 0.150%, 0.175%, 0.200%, 0.210%, 0.211%, 0.212%, 0.213%, 0.214%, 0.215%, 022%, 0.250%, 0.275%, 0.300%. The concentration of sodium phosphate monobasic in the final solution may be 0.010-0.050%, with non-limiting examples of 0.010%, 0.015%, 0.020%, 0.021%, 0.022%, 0.023%, 0.024%, 0.025%, 0.026%, 0.027%, 0.028%, 0.029%, 0.030%, 0.035%, 0.040%, 0.045%, or 0.050%. The concentration of poloxamer 188 (Pluronic acid (F-68)) may be 0.0001%-1%. As non-limiting examples, the concentration of poloxamer 188 (Pluronic acid (F-68)) may be 0.0001%, 0.0005%, 00.0010%, 00.0050%, 0.01%, 0.05%, 0.1%, 0.5%, or 1%. The final solution may have a pH of 6.8-7.7. Non-limiting examples for the pH of the final solution include a pH of 6.8, 6.9, 7.0, 7.1, 7.2, 7.3, 7.4, 7.5, 7.6, or 7.7.

Relative amounts of active ingredient (e.g. antibody), pharmaceutically acceptable excipients, and/or any additional ingredients in pharmaceutical compositions in accordance with the present disclosure may vary, depending upon the identity, size, and/or condition of subjects being treated and further depending upon route of administration. For example, compositions may include between 0.1% and 99% (w/w) of active ingredient. By way of example, compositions may include between 0.1% and 100%, e.g., between 0.5 and 50%, between 1-30%, between 5-80%, or at least 80% (w/w) active ingredient.

According to the present disclosure, compounds may be formulated for CNS delivery. Agents that cross the brain blood barrier may be used. For example, some cell penetrating peptides that can target molecules to the brain blood barrier endothelium may be used for formulation (e.g., Mathupala, Expert Opin Ther Pat., 2009, 19, 137-140; the content of which is incorporated herein by reference in its entirety).

### Excipients and Diluents

In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by the United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparation are known in the art (see Remington: The Science and Practice of Pharmacy, 21st Edition, A. R. Gennaro, Lippincott, Williams & Wilkins, Baltimore, MD, 2006; incorporated herein by reference in its entirety). The use of conventional excipient media may be contemplated within the scope of the present disclosure, except insofar as any conventional excipient media may be incompatible with certain substances or their derivatives, such as by producing any undesirable biological effects or otherwise interacting in a deleterious manner with any other component(s) of pharmaceutical compositions of the present disclosure.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

### Inactive Ingredients

In some embodiments, formulations of the present disclosure may include at least one inactive ingredient. As used herein, the term “inactive ingredient” refers to an agent that does not contribute to the activity of a pharmaceutical composition. In some embodiments, all, none or some of the inactive ingredients which may be used in formulations of the present disclosure may be approved by the US Food and Drug Administration (FDA).

Formulations disclosed herein may include cations or anions. Formulations may include Zn²⁺, Ca²⁺, Cu²⁺, Mn²⁺, Mg⁺, or combinations thereof. As a non-limiting example, formulations may include polymers and complexes with metal cations (See e.g., U.S. Pat. Nos. 6,265,389 and 6,555,525, each of which is herein incorporated by reference in its entirety).

### IV. Administration and Dosing

### Administration

Compounds and compositions (e.g., AAV particles) of the present disclosure may be administered by any delivery route which results in a therapeutically effective outcome.

In some embodiments, compositions may be administered in a way which allows them to cross the blood-brain barrier, vascular barrier, or other epithelial barrier. Compounds and compositions of the present disclosure may be administered in any suitable form, including, but not limited to, as a liquid solution, as a suspension, or as a solid form suitable for liquid solution or suspension in a liquid solution.

In some embodiments, delivery to a subject may be via a single route administration. In some embodiments, delivery to a subject may be via multi-site route of administration. Administration may include a bolus infusion. Administration may include sustained delivery over a period of minutes, hours, or days. Administration by infusion may include an infusion rate that may be changed depending on the subject, distribution, formulation, or other delivery parameter. Administration may be by more than one route of administration. As non-limiting examples, combination administrations may include intrathecal and intracerebroventricular administration, or intravenous and intraparenchymal administration.

### Intravenous Administration

Compounds and compositions of the present disclosure may be administered to a subject by systemic administration. In some embodiments, systemic administration may include intravenous administration. Systemic administration may include intraarterial administration.

Compounds and compositions of the present disclosure may be administered to a subject by intravenous administration. In some embodiments, intravenous administration may be achieved by subcutaneous delivery. In some embodiments, the AAV particle is administered to the subject via focused ultrasound (FUS), e.g., coupled with the intravenous administration of microbubbles (FUS-MB), or MRI-guided FUS coupled with intravenous administration, e.g., as described in Terstappen et al. (Nat Rev Drug Discovery, doi.org/10.1038/s41573-021-00139-y (2021)), Burgess et al. (Expert Rev Neurother. 15(5): 477-491 (2015)), and/or Hsu et al. (PLOS One 8(2): 1-8), the contents of which are incorporated herein by reference in its entirety. In some embodiments, the AAV particle is administered to the subject intravenously. Intravenous administration may be achieved by a tail vein injection (e.g., in a mouse model). Intravenous administration may be achieved by retro-orbital injection.

### Administration to the CNS

Compounds and compositions of the present disclosure may be administered to a subject by direct injection into the brain. As a non-limiting example, the brain delivery may be by intrahippocampal administration. Administration may be by intraparenchymal administration. In some embodiments, the intraparenchymal administration is to tissue of the central nervous system. Administration may be by intracranial delivery (See, e.g., U.S. Pat. No. 8,119,611; the content of which is incorporated herein by reference in its entirety). Administration may be by injection into the CSF pathway. Non-limiting examples of delivery to the CSF pathway include intrathecal and intracerebroventricular (e.g., intracistemal magna—ICM) administration. Administration to the brain may be by systemic delivery. As a non-limiting example, the systemic delivery may be by intravascular administration. As a non-limiting example, the systemic or intravascular administration may be intravenous. Administration may be by intraocular delivery route. A non-limiting example of intraocular administration includes an intravitreal injection.

### Intramuscular Administration

In some embodiments, the AAV particles may be delivered by intramuscular administration. Whilst not wishing to be bound by theory, the multi-nucleated nature of muscle cells provides an advantage to gene transduction subsequent to AAV delivery. Cells of the muscle are capable of expressing recombinant proteins with the appropriate post-translational modifications. The enrichment of muscle tissue with vascular structures allows for transfer to the blood stream and whole-body delivery. Examples of intramuscular administration include systemic (e.g., intravenous), subcutaneous or directly into the muscle. In some embodiments, more than one injection is administered.

In some embodiments, the AAV particles of the present disclosure may be delivered by intramuscular delivery route. (See, e.g., U.S. Pat. No. 6,506,379; the content of which is incorporated herein by reference in its entirety). Non-limiting examples of intramuscular administration include an intravenous injection or a subcutaneous injection.

In some embodiments, the AAV particles of the present disclosure are administered to a subject and transduce muscle of a subject. As a non-limiting example, the AAV particles are administered by intramuscular administration.

In some embodiments, the AAV particles of the present disclosure may be administered to a subject by subcutaneous administration.

In some embodiments, the intramuscular administration is via systemic delivery.

In some embodiments, the intramuscular administration is via intravenous delivery.

In some embodiments, the intramuscular administration is via direct injection to the muscle.

In some embodiments, the muscle is transduced by administration, and this is referred to as intramuscular administration.

In some embodiments, the intramuscular delivery comprises administration at one site.

In some embodiments, the intramuscular delivery comprises administration at more than one site. In some embodiments, the intramuscular delivery comprises administration at two sites. In some embodiments, the intramuscular delivery comprises administration at three sites. In some embodiments, the intramuscular delivery comprises administration at four sites. In some embodiments, the intramuscular delivery comprises administration at more than four sites.

In some embodiments, intramuscular delivery is combined with at least one other method of administration.

In some embodiments, the AAV particles that may be administered to a subject by peripheral injections. Non-limiting examples of peripheral injections include intraperitoneal, intramuscular, intravenous, conjunctival, or joint injection. It was disclosed in the art that the peripheral administration of AAV vectors can be transported to the central nervous system, for example, to the motor neurons (e.g., U.S. Patent Publication Nos. US20100240739 and US20100130594; the content of each of which is incorporated herein by reference in their entirety).

In some embodiments, the AAV particles of the present disclosure may be administered to a subject by intraparenchymal administration. In some embodiments, the intraparenchymal administration is to muscle tissue.

In some embodiments, the AAV particles of the present disclosure are delivered as described in Bright et al 2015 (Neurobiol Aging. 36(2):693-709), the contents of which are herein incorporated by reference in their entirety.

In some embodiments, the AAV particles of the present disclosure are administered to the gastrocnemius muscle of a subject. In some embodiments, the AAV particles of the present disclosure are administered to the bicep femorii of the subject. In some embodiments, the AAV particles of the present disclosure are administered to the tibialis anterior muscles. In some embodiments, the AAV particles of the present disclosure are administered to the soleus muscle.

### Depot Administration

As described herein, in some embodiments, pharmaceutical compositions, AAV particles of the present disclosure are formulated in depots for extended release. Generally, specific organs or tissues (“target tissues”) are targeted for administration.

In some aspects, pharmaceutical compositions, AAV particles of the present disclosure are spatially retained within or proximal to target tissues. Provided are methods of providing pharmaceutical compositions, AAV particles, to target tissues of mammalian subjects by contacting target tissues (which comprise one or more target cells) with pharmaceutical compositions, AAV particles, under conditions such that they are substantially retained in target tissues, meaning that at least 10, 20, 30, 40, 50, 60, 70, 80, 85, 90, 95, 96, 97, 98, 99, 99.9, 99.99 or greater than 99.99% of the composition is retained in the target tissues. Advantageously, retention is determined by measuring the amount of pharmaceutical compositions, AAV particles, that enter one or more target cells. For example, at least 1%, 5%, 10%, 20%, 30%, 40%, 50%, 60%, 70%, 80%, 85%, 90%, 95%, 96%, 97%, 98%, 99%, 99.9%, 99.99%, or greater than 99.99% of pharmaceutical compositions, AAV particles, administered to subjects are present intracellularly at a period of time following administration. For example, intramuscular injection to mammalian subjects may be performed using aqueous compositions comprising pharmaceutical compositions, AAV particles of the present disclosure and one or more transfection reagents, and retention is determined by measuring the amount of pharmaceutical compositions, AAV particles, present in muscle cells.

Certain aspects are directed to methods of providing pharmaceutical compositions, AAV particles of the present disclosure to a target tissues of mammalian subjects, by contacting target tissues (comprising one or more target cells) with pharmaceutical compositions, AAV particles under conditions such that they are substantially retained in such target tissues. Pharmaceutical compositions, AAV particles comprise enough active ingredient such that the effect of interest is produced in at least one target cell. In some embodiments, pharmaceutical compositions, AAV particles generally comprise one or more cell penetration agents, although “naked” formulations (such as without cell penetration agents or other agents) are also contemplated, with or without pharmaceutically acceptable carriers.

### Dose and Regimen

The present disclosure provides methods of administering compounds and compositions in accordance with the disclosure to a subject in need thereof. Administration may be in any amount and by any route of administration effective for preventing, treating, managing, or diagnosing diseases, disorders, and/or conditions. The exact amount required may vary from subject to subject, depending on species, age, general condition of the subject, severity of disease, particular composition, mode of administration, mode of activity, and the like. Subjects may be, but are not limited to, humans, mammals, or animals. Compositions may be formulated in unit dosage form for ease of administration and uniformity of dosage. It will be understood, however, that the total daily usage of compositions of the present disclosure may be decided by an attending physician within the scope of sound medical judgment. Specific therapeutically effective, prophylactically effective, or appropriate diagnostic dose levels for any particular individual may vary depending upon a variety of factors including the disorder being treated and severity of the disorder; the activity of specific payloads employed; specific compositions employed; age, body weight, general health, sex, and diet of patients; time of administration, route of administration, and rate of excretion of compounds and compositions employed; duration of treatment; drugs used in combination or coincidental with compounds and compositions employed; and like factors well known in the medical arts.

In some embodiments, compounds and compositions in accordance with the present disclosure may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, or prophylactic, effect.

In some embodiments, the desired dosage may be delivered using multiple administrations (e.g., two, three, four, or more than four administrations). When multiple administrations are employed, split dosing regimens such as those described herein may be used. As used herein, a “split dose” is the division of “single unit dose” or total daily dose into two or more doses, e.g., two or more administrations of the “single unit dose”. As used herein, a “single unit dose” is a dose of any therapeutic administered in one dose/at one time/single route/single point of contact, i.e., single administration event.

Compounds and compositions of the present disclosure may be administered as a “pulse dose” or as a “continuous flow.” As used herein, a “pulse dose” is a series of single unit doses of any therapeutic agent administered with a set frequency over a period of time. As used herein, a “continuous flow” is a dose of therapeutic agent administered continuously for a period of time in a single route/single point of contact, i.e., continuous administration event. A total daily dose, an amount given or prescribed in a 24-hour period, may be administered by any of these methods, or as a combination of these methods, or by any other methods suitable for pharmaceutical administration.

In some embodiments, delivery of AAV particles may comprise a total dose between about 1×10⁶ VG and about 1×10¹⁶ VG. In some embodiments, delivery may comprise a total dose of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 1.9×10¹⁰, 2×10¹⁰, 3×10¹⁰, 3.73×10¹⁰, 4×10¹⁰, 5×10¹⁰, 6×10¹⁰, 7×10¹⁰, 8×10¹⁰, 9×10¹⁰, 1×10¹¹, 2×10¹¹, 2.5×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁵, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG. As a non-limiting example, the total dose is 1×10¹³VG. As another non-limiting example, the total dose is 2.1×10¹²VG.

In some embodiments, delivery of AAV particles may comprise a composition concentration between about 1×10⁶ VG/mL and about 1×10¹⁶ VG/mL. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/mL. In some embodiments, the delivery comprises a composition concentration of 1×10¹³ VG/mL. In some embodiments, the delivery comprises a composition concentration of 2.1×10¹² VG/mL.

### Combinations

Compounds and compositions of the present disclosure may be used in combination with one or more other therapeutic, prophylactic, research or diagnostic agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of pharmaceutical, prophylactic, research, or diagnostic compositions in combination with agents that may improve their bioavailability, reduce and/or modify their metabolism, inhibit their excretion, and/or modify their distribution within the body.

### V. Methods and Uses of the Compositions

In some embodiments, the present disclosure provides methods related to using and evaluating compounds and compositions for therapeutic and diagnostic applications.

### Therapeutic Applications

In some embodiments, methods of the present disclosure include methods of treating therapeutic indications using compounds and/or compositions disclosed herein. As used herein, the term “therapeutic indication” refers to any symptom, condition, disorder, or disease that may be alleviated, stabilized, improved, cured, or otherwise addressed by some form of treatment or other therapeutic intervention. In some embodiments, methods of the present disclosure include treating therapeutic indications by administering antibodies disclosed herein.

As used herein the terms “treat,” “treatment,” and the like, refer to relief from or alleviation of pathological processes. In the context of the present disclosure insofar as it relates to any of the other conditions recited herein below, the terms “treat,” “treatment,” and the like mean to relieve or alleviate at least one symptom associated with such condition, or to slow or reverse the progression or anticipated progression of such condition.

By “lower” or “reduce” in the context of a disease marker or symptom is meant a significant decrease in such a level, often statistically significant. The decrease may be, for example, at least 10%, at least 20%, at least 30%, at least 40% or more, and is preferably down to a level accepted as within the range of normal for an individual without such a disorder.

By “increase” or “raise” in the context of a disease marker or symptom is meant a significant rise in such level, often statistically significant. The increase may be, for example, at least 10%, at least 20%, at least 30%, at least 40% or more, and is preferably up to a level accepted as within the range of normal for an individual without such disorder.

Efficacy of treatment or amelioration of disease can be assessed, for example by measuring disease progression, disease remission, symptom severity, reduction in pain, quality of life, dose of a medication required to sustain a treatment effect, level of a disease marker or any other measurable parameter appropriate for a given disease being treated or targeted for prevention. It is well within the ability of one skilled in the art to monitor efficacy of treatment or prevention by measuring any one of such parameters, or any combination of parameters. In connection with the administration of a compound or composition described herein, “effective against” a disease or disorder indicates that administration in a clinically appropriate manner results in a beneficial effect for at least a fraction of patients, such as an improvement of symptoms, a cure, a reduction in disease load, reduction in protein aggregation, reduction in neurofibrillary tangles, reduction in neurodegeneration, extension of life, improvement in quality of life, or other effect generally recognized as positive by medical doctors familiar with treating the particular type of disease or disorder.

A treatment or preventive effect is evident when there is a significant improvement, often statistically significant, in one or more parameters of disease status, or by a failure to worsen or to develop symptoms where they would otherwise be anticipated. As an example, a favorable change of at least 10% in a measurable parameter of disease, and preferably at least 20%, 30%, 40%, 50% or more may be indicative of effective treatment. Efficacy for a given compound or composition may also be judged using an experimental animal model for the given disease as known in the art. When using an experimental animal model, efficacy of treatment is evidenced when a statistically significant modulation in a marker or symptom is observed.

Compounds of the present disclosure and additional therapeutic agents can be administered in combination. Such combinations may be in the same composition, or the additional therapeutic agents can be administered as part of a separate composition or by another method described herein.

In some embodiments, therapeutic indications that may be addressed by methods of the present disclosure include neurological indications. As used herein, a “neurological indication” refers to any therapeutic indication relating to the central nervous system (CNS). Methods of treating neurological indications according to the present disclosure may include administering compounds (e.g., antibodies) and/or compositions described herein. Neurological indications may include neurological diseases and/or disorders involving irregular expression or aggregation of tau. Such indications may include, but are not limited to mild cognitive impairment (MCI), neurodegenerative disease, Alzheimer's disease (AD), frontotemporal dementia and parkinsonism linked to chromosome 17 (FTDP-17), frontotemporal lobar degeneration (FTLD), frontotemporal dementia (FTD), chronic traumatic encephalopathy (CTE), progressive supranuclear palsy (PSP), Down's syndrome, Pick's disease, corticobasal degeneration (CBD), corticobasal syndrome, amyotrophic lateral sclerosis (ALS), a prion disease, Creutzfeldt-Jakob disease (CJD), multiple system atrophy, tangle-only dementia, stroke, and progressive subcortical gliosis.

In some embodiments, an antibody that binds to tau described herein or an AAV particle comprising a payload, e.g., an antibody that binds to tau described herein, can be used to treat a traumatic brain injury (TBI), e.g., as described in Edwards et al. “Traumatic Brain Injury Induces Tau Aggregation and Spreading,” J. Neurotrauma, 2020, 37(1):80-92, the contents of which are hereby incorporated by reference in their entirety.

In some embodiments, methods of treating neurological diseases and/or disorders in a subject in need thereof may include one or more of the steps of: (1) deriving, generating, and/or selecting an anti-tau antibody or fragment or composition thereof; and (2) administering the anti-tau antibody or fragment or composition thereof to the subject. Administration to the subject may slow, stop, or reverse disease progression. As a non-limiting example, disease progression may be measured by cognitive tests such as, but not limited to, the Mini-Mental State Exam (MMSE); imaging tests (e.g., positron emission tomography (PET) scan; or PET scan in combination with Braak neuropathological staging and/or serum biomarker staining (e.g., ¹⁸F-flortaucipir, plasma ptau 181, or ¹⁸F-PM-PBB3), or other similar diagnostic tool(s), known to those skilled in the art. As another non-limiting example, disease progression may be measured by change in the pathological features of the brain, CSF or other tissues of the subject, such as, but not limited to a decrease in levels of tau (either soluble or insoluble). In some embodiments, levels of insoluble hyperphosphorylated tau are decreased. In some embodiments, levels of soluble tau are decreased. In some embodiments, both soluble and insoluble tau are decreased. In some embodiments, levels of insoluble hyperphosphorylated tau are increased. In some embodiments, levels of soluble tau are increased. In some embodiments, both insoluble and soluble tau levels are increased. In some embodiments, neurofibrillary tangles are decreased in size, number, density, or combination thereof. In another embodiment, neurofibrillary tangles are increased in size, number, density or combination thereof.

In some embodiments, administration of an anti-tau antibody or a viral genome encoding a anti-tau antibody described herein results in a reduction of tau pathology, e.g., a decrease in a biomarker of tau pathology (e.g., ¹⁸F-flortaucipir, plasma ptau 181, or ¹⁸F-PM-PBB3), e.g., as measured by a PET scan or PET scan in combination with Braak neuropathological staging and/or serum biomarker staining.

### Neurodegeneration

Neurodegenerative disease refers to a group of conditions characterized by progressive loss of neuronal structure and function, ultimately leading to neuronal cell death. Neurons are the building blocks of the nervous system(s) and are generally not able to reproduce and/or be replaced, and therefore neuron damage and/or death is especially devastating. Other, non-degenerating diseases that lead to neuronal cell loss, such as stroke, have similarly debilitating outcomes. Targeting molecules that contribute to deteriorating cell structure or function may prove beneficial generally for treatment of neurological indications, including neurodegenerative disease and stroke.

Certain molecules are believed to have inhibitory effects on neurite outgrowth, contributing to the limited ability of the central nervous system to repair damage. Such molecules include, but are not limited to, myelin associated proteins, such as, but not limited to, RGM (Repulsive guidance molecule), NOGO (Neurite outgrowth inhibitor), NOGO receptor, MAG (myelin associated glycoprotein), and MAI (myelin associated inhibitor). In some embodiments, anti-tau antibodies of the present disclosure may be utilized to target the aforementioned antigens (e.g., neurite outgrowth inhibitors).

Many neurodegenerative diseases are associated with aggregation of misfolded proteins, including, but not limited to, alpha synuclein, tau (as in tauopathies), amyloid β, prion proteins, TAR DNA binding protein 43 (TDP-43), and huntingtin (see, e.g. De Genst et al., 2014, Biochim Biophys Acta; 1844(11):1907-1919, and Yu et al., 2013, Neurotherapeutics.; 10(3): 459-472, references therein, all of which are herein incorporated by reference in their entirety). The aggregation results from disease-specific conversion of soluble proteins to an insoluble, highly ordered fibrillary deposit. This conversion is thought to prevent the proper disposal or degradation of misfolded proteins, thereby leading to further aggregation. Conditions associated with alpha synuclein misfolding and aggregation are referred to as “synucleinopathies.” In some embodiments, anti-tau antibodies of the present disclosure may be utilized to target misfolded or aggregated proteins.

### Alzheimer's Disease

Alzheimer Disease (AD) is a debilitating neurodegenerative disease currently afflicting more than 35 million people worldwide, with that number expected to double in coming decades. Symptomatic treatments have been available for many years but these treatments do not address the underlying pathophysiology. Recent clinical trials using these and other treatments have largely failed and, to date, no known cure has been identified.

The AD brain is characterized by the presence of two forms of pathological aggregates, the extracellular plaques composed of β-amyloid (Aβ) and the intracellular neurofibrillary tangles (NFT) comprised of hyperphosphorylated microtubule associated protein tau. Based on early genetic findings, β-amyloid alterations were thought to initiate disease, with changes in tau considered downstream. Thus, most clinical trials have been Aβ-centric. Although no mutations of the tau gene have been linked to AD, such alterations have been shown to result in a family of dementias known as tauopathies, demonstrating that changes in tau can contribute to neurodegenerative processes. Tau is normally a soluble protein known to associate with microtubules based on the extent of its phosphorylation. Hyperphosphorylation of tau depresses its binding to microtubules and microtubule assembly activity. In tauopathies, the tau becomes hyperphosphorylated, misfolds and aggregates as NFT of paired helical filaments (PHF), twisted ribbons or straight filaments. In AD, NFT pathology, rather than plaque pathology, correlates more closely with neuropathological markers such as neuronal loss, synaptic deficits, severity of disease and cognitive decline. NFT pathology marches through the brain in a stereotyped manner and animal studies suggest a trans-cellular propagation mechanism along neuronal connections.

Several approaches have been proposed for therapeutically interfering with progression of tau pathology and preventing the subsequent molecular and cellular consequences. Given that NFT are composed of a hyperphosphorylated, misfolded and aggregated form of tau, interference at each of these stages has yielded the most avidly pursued set of targets. Introducing agents that limit phosphorylation, block misfolding or prevent aggregation have all generated promising results. Passive and active immunization with late stage anti-phospho-tau antibodies in mouse models have led to dramatic decreases in tau aggregation and improvements in cognitive parameters. It has also been suggested that introduction of anti-tau antibodies can prevent the trans-neuronal spread of tau pathology.

In some embodiments, anti-tau antibodies of the present disclosure may be used according to methods presented herein to treat subjects suffering from AD and other tauopathies. In some cases, methods of the present disclosure may be used to treat subjects suspected of developing AD or other tauopathies.

### Frontotemporal Dementia and Parkinsonism Linked to Chromosome 17 (FTDP-17)

Although Alzheimer's disease is, in part, characterized by the presence of tau pathology, no known mutations in the tau gene have been causally linked to the disease. Mutations in the tau gene have been shown to lead to an autosomal dominantly inherited tauopathy known as frontotemporal dementia and parkinsonism linked to chromosome 17 (FTDP-17) and demonstrate that alterations in tau can lead to neurodegenerative changes in the brain. Mutations in the tau gene that lead to FTDP-17 are thought to influence splicing patterns, thereby leading to an elevated proportion of tau with four microtubule binding domains (rather than three). These molecules are considered to be more amyloidogenic, meaning they are more likely to become hyperphosphorylated and more likely to aggregate into NFT (Hutton, M. et al., 1998, Nature 393(6686):702-5, the contents of which are herein incorporated by reference in their entirety). Although physically and behaviorally, FTDP-17 patients can appear quite similar to Alzheimer's disease patients, at autopsy FTDP-17 brains lack the prominent Aβ plaque pathology of an AD brain (Gotz, J. et al., 2012, British Journal of Pharmacology 165(5):1246-59, the contents of which are herein incorporated by reference in their entirety). Therapeutically targeting the aggregates of tau protein may ameliorate and prevent degenerative changes in the brain and potentially lead to improved cognitive ability.

As of today, there is no treatment to prevent, slow the progression, or cure FTDP-17. Medication may be prescribed to reduce aggressive, agitated or dangerous behavior. There remains a need for therapy affecting the underlying pathophysiology, such as antibody therapies targeting tau protein.

In some embodiments, anti-tau antibodies of the present disclosure may be used to treat subjects suffering from FTDP-17. In some cases, methods of the present disclosure may be used to treat subjects suspected of developing FTDP-17.

### Chronic Traumatic Encephalopathy

Unlike the genetically linked tauopathies, chronic traumatic encephalopathy is a degenerative tauopathy linked to repeated head injuries. The disease was first described in boxers whom behaved “punch drunk” and has since been identified primarily in athletes that play American football, ice hockey, wrestling and other contact sports. The brains of those suffering from CTE are characterized by distinctive patterns of brain atrophy accompanied by accumulation of hyperphosphorylated species of aggregated tau in NFT. In CTE, pathological changes in tau are accompanied by a number of other pathobiological processes, such as inflammation (Daneshvar, D. H. et al., 2015 Mol Cell Neurosci 66(Pt B): 81-90, the contents of which are herein incorporated by reference in their entirety). Targeting the tau aggregates may provide reprieve from the progression of the disease and may allow cognitive improvement.

As of today, there is no medical therapy to treat or cure CTE. The condition is only diagnosed after death, due to lack of in vivo techniques to identify CTE specific biomarkers. There remains a need for therapy affecting the underlying pathophysiology, such as antibody therapies targeting tau protein.

In some embodiments, anti-tau antibodies of the present disclosure may be used to treat subjects having or suffering from CTE. In some cases, methods of the present disclosure may be used to treat subjects suspected of developing CTE. In some embodiments, anti-tau antibodies of the present disclosure may be used to treat subjects having a traumatic brain injury (TBI), e.g., CTE.

### Prion Diseases

Prion diseases, also known as transmissible spongiform encephalopathies (TSEs), are a group of rare progressive conditions affecting the nervous system. The related conditions are rare and are typically caused by mutations in the PRNP gene which enables production of the prion protein. Gene mutations lead to an abnormally structured prion protein. Alternatively, the abnormal prion may be acquired by exposure from an outside source, e.g. by consumption of beef products containing the abnormal prion protein. Abnormal prions are misfolded, causing the brain tissue to degenerate rapidly. Prion diseases include, but are not limited to, Creutzfeldt-Jakob disease (CJD), Gerstmann-Sträussler-Scheinker syndrome (GSS), fatal insomnia (FFI), variably protease-sensitive prionopathy (VPSPr), and kuru. Prion diseases are rare. Approximately 350 cases of prion diseases are diagnosed in the US annually.

CJD is a degenerative brain disorder characterized by problems with muscular coordination, personality changes including mental impairment, impaired vision, involuntary muscle jerks, weakness and eventually coma. The most common categories of CJD are sporadic, hereditary due to a genetic mutation, and acquired. Sporadic CJD is the most common form affecting people with no known risk factors for the disease. The acquired form of CJD is transmitted by exposure of the brain and nervous system tissue to the prion. As an example, variant CJD (vCDJ) is linked to a bovine spongiform encephalopathy (BSE), also known as a ‘mad cow’ disease. CJD is fatal and patients typically die within one year of diagnosis.

Prion diseases are associated with an infectious agent consisting of an alternative conformational isoform of the prion protein, PrPSc. PrPSc replication is considered to occur through an induction of the infectious prion in the normal prion protein (PrPC). The replication occurs without a nucleic acid.

As of today, there is no therapy to manage or cure CJD, or other prion diseases. Typically, treatment is aimed at alleviating symptoms and increasing comfort of the patient, e.g. with pain relievers. There remains a need for therapy affecting the underlying pathophysiology.

In some embodiments, anti-tau antibodies of the present disclosure may be used to treat subjects suffering from a prion disease. In some cases, methods of the present disclosure may be used to treat subjects suspected of developing a prion disease.

### Diagnostic Applications

In some embodiments, compounds (e.g., antibodies) and compositions of the present disclosure may be used as diagnostics. Anti-tau antibodies may be used to identify, label, or stain cells, tissues, organs, etc. expressing tau proteins. Anti-tau antibodies may be used to identify tau proteins present in tissue sections (e.g., histological tissue sections), including tissue known or suspected of having tau protein aggregates. Such antibodies may in some cases be used to identify subjects with neurological diseases and/or disorders. Tissue sections may be from CNS tissue.

In some embodiments, diagnostic methods of the present disclosure may include the analysis of one or more cells or tissues using immunohistochemical techniques. Such methods may include the use of one or more of any of the anti-tau antibodies described herein. Immunohistochemical methods may include staining tissue sections to determine the presence and/or level of one or more tau proteins or other markers. Tissue sections may be derived from subject CNS tissue (e.g., patient CNS, animal CNS, and CNS from animal models of disease). Tissue sections may come from formalin-fixed or unfixed fresh frozen tissues. In some cases, tissue sections come from formalin fixed paraffin-embedded (FFPE) tissues. Anti-tau antibodies described herein may be used as primary antibodies. Primary antibodies are used to contact tissue sections directly and bind to target epitopes. Primary antibodies may be directly conjugated with a detectable label or may be detected through the use of a detection agent such as a secondary antibody. In some embodiments, primary antibodies or detection agents include an enzyme that can be used to react with a substrate to generate a visible product (e.g., precipitate). Such enzymes may include, but are not limited to horse radish peroxidase, alkaline phosphatase, beta-galactosidase, and catalase.

Anti-tau antibodies described herein may be used according to immunohistochemical methods of the present disclosure to detect tau proteins in tissues or cells. In some cases, these antibodies are used to detect and/or determine the level of tau proteins in tissues. Levels of anti-tau antibodies used in immunohistochemical staining techniques may be varied to increase visible staining or to decrease background levels of staining. In some embodiments, antibody concentrations of from about 0.01 μg/ml to about 50 μg/ml are used. For example, antibody concentrations of from about 0.01 μg/ml to about 1 μg/ml, from about 0.05 μg/ml to about 5 μg/ml, from about 0.1 μg/ml to about 3 μg/ml, from about 1 μg/ml to about 10 μg/ml, from about 2 μg/ml to about 20 μg/ml, from about 3 μg/ml to about 25 μg/ml, from about 4 μg/ml to about 30 μg/ml, or from about 5 μg/ml to about 50 μg/ml may be used.

Levels and/or identities of tau proteins may be determined according to any methods known in the art for identifying proteins and/or quantitating protein levels. In some embodiments, such methods may include, but are not limited to mass spectrometry, array analysis (e.g., antibody array or protein array), Western blotting, flow cytometry, immunoprecipitation, surface plasmon resonance analysis, and ELISA. Tau proteins may in some cases be immunoprecipitated from samples prior to analysis. Such immunoprecipitation may be carried out using anti-tau antibodies disclosed herein. In some embodiments, tau proteins are immunoprecipitated from biological samples using anti-tau antibodies and then identified and/or quantitated using mass spectrometry.

In some embodiments, treatments are informed by diagnostic information generated using anti-tau antibodies. Accordingly, the present disclosure provides methods of treating neurological diseases and/or disorders that include obtaining a sample from a subject, diagnosing one or more neurological diseases and/or disorders using an anti-tau antibody, and administering a treatment selected based on the diagnosis. Such treatments may include treatment with anti-tau antibodies. Anti-tau antibodies administered according to such methods may include any of those described herein.

In some embodiments, the present disclosure provides methods of detecting and/or quantifying tau proteins in samples through the use of capture and detection antibodies. As used herein, a “capture antibody” is an antibody that binds an analyte in a way that it may be isolated or detected. Capture antibodies may be associated with surfaces or other carriers (e.g., beads). Detection antibodies are antibodies that facilitate observation of the presence or absence of an analyte. According to some methods of detecting and/or quantifying tau proteins, both capture antibodies and detection antibodies bind to tau proteins. Capture and detection antibodies may bind to different epitopes or regions of tau proteins to avoid competition for binding. In some embodiments, detection antibodies may be conjugated with a detectable label for direct detection. In some embodiments, binding of detection antibodies may be assessed using a secondary antibody that binds to a constant domain of the detection antibody or to a detectable label of the detection antibody. Capture, detection, and/or secondary antibodies may be derived from different species. This may prevent secondary antibodies from binding to both capture and detection antibodies.

### VI. Kits and Devices

### Kits

In some embodiments, compounds and composition of the present disclosure may be included in a kit. Such compounds and compositions may include anti-tau antibodies disclosed herein. In a non-limiting example, kits may include reagents for generating anti-tau antibodies, including tau protein antigens. Kits may include additional reagents and/or instructions for use, e.g., for creating or synthesizing anti-tau antibodies. Kits may include one or more buffers. Kits may include additional components, for example, solid supports or substrates for antibody or antigen attachment.

In some embodiments, the present disclosure includes kits for screening, monitoring, and/or diagnosis of a subject that include one or more anti-tau antibodies. Such kits may be used alone or in combination with one or more other methods of screening, monitoring, and/or diagnosis. Kits may include one or more of a buffer, a biological standard, a secondary antibody, a detection reagent, and a composition for sample pre-treatment (e.g., for antigen retrieval, blocking, etc.).

Kit components may be packaged. In some embodiments, kit components are packaged in aqueous media or in lyophilized form. Packaging may include one or more vial, test tube, flask, bottle, syringe or other container into which a component may be placed and/or suitably aliquoted. Where there are multiple kit components (labeling reagent and label may be packaged together), kits may include second, third or other additional containers into which additional components may be separately placed.

When kit components are provided in one and/or more liquid solutions, liquid solutions may be aqueous. Liquid solutions may be provided sterile. Kit components may be provided as dried powder(s). Dried powder components may be provided for reconstitution by kit users, e.g., by addition of suitable solvent. Solvents may also be provided in kits in one or more separate containers. In some embodiments, labeling dyes are provided in dried powder format.

Kits may include instructions for employing kit components as well other reagents not included in the kit. Instructions may include variations that can be implemented.

### Devices

Any of the compounds and compositions described herein may be combined with, coated onto, or embedded in, or delivered by a device. Devices may include, but are not limited to, implants, stents, bone replacements, artificial joints, valves, pacemakers, or other implantable therapeutic devices.

### VII. Definitions

At various places in the present specification, substituents of compounds of the present disclosure are disclosed in groups or in ranges. It is specifically intended that the present disclosure include each and every individual sub combination of the members of such groups and ranges.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

About: As used herein, the term “about” means+/−10% of the recited value.

AAV Particle: As used herein, an “AAV particle” refers to a particle or a virion comprising an AAV capsid, e.g., an AAV capsid variant, and a polynucleotide, e.g., a viral genome or a vector genome. In some embodiments, the viral genome of the AAV particle comprises at least one payload region and at least one ITR. In some embodiments, an AAV particle of the disclosure is an AAV particle comprising an AAV variant. In some embodiments, the AAV particle is capable of delivering a nucleic acid, e.g., a payload region, encoding a payload to cells, typically, mammalian, e.g., human, cells. In some embodiments, an AAV particle of the present disclosure may be produced recombinantly. In some embodiments, an AAV particle may be derived from any serotype, described herein or known in the art, including combinations of serotypes (e.g., “pseudotyped” AAV) or from various genomes (e.g., single stranded or self-complementary). In some embodiments, the AAV particle may be replication defective and/or targeted. It is to be understood that reference to the AAV particle of the disclosure also includes pharmaceutical compositions thereof, even if not explicitly recited.

Administered in combination: As used herein, the term “administered in combination” or “combined administration” means that two or more agents are administered to a subject at the same time or within an interval such that there may be an overlap of an effect of each agent on the patient. In some embodiments, they are administered within about 60, 30, 15, 10, 5, or 1 minute of one another. In some embodiments, the administrations of the agents are spaced sufficiently closely together such that a combinatorial (e.g., a synergistic) effect is achieved.

Amelioration: As used herein, the term “amelioration” or “ameliorating” refers to a lessening of severity of at least one indicator of a condition or disease. For example, in the context of neurodegeneration disorder, amelioration includes the reduction of neuron loss.

Approximately: As used herein, the term “approximately” or “about,” as applied to one or more values of interest, refers to a value that is similar to a stated reference value. When referring to a measurable value such as an amount, a temporal duration, and the like, the term is meant to encompass is meant to encompass variations of ±20% or in some instances ±10%, or in some instances ±5%, or in some instances ±1%, or in some instances ±0.1

Associated with: As used herein, the terms “associated with,” “conjugated,” “linked,” “attached,” and “tethered,” when used with respect to two or more entities, means that the entities are physically associated or connected with one another, either directly or via a linker, to form a structure that is sufficiently stable so that the entities remain physically associated, e.g., under working conditions, e.g., under physiological conditions. An “association” need not be through covalent chemical bonding and may include other forms of association or bonding sufficiently stable such that the “associated” entities remain physically associated, e.g., ionic bonding, hydrostatic bonding, hydrophobic bonding, hydrogen bonding, or hybridization-based connectivity.

Capsid: As used herein, the term “capsid” refers to the exterior, e.g., a protein shell, of a virus particle, e.g., an AAV particle, that is substantially (e.g., >50%, >90%, or 100%) protein. In some embodiments, the capsid is an AAV capsid comprising an AAV capsid protein described herein, e.g., a VP1, VP2, and/or VP3 polypeptide. The AAV capsid protein can be a wild-type AAV capsid protein or a variant, e.g., a structural and/or functional variant from a wild-type or a reference capsid protein, referred to herein as an “AAV capsid variant.” In some embodiments, the AAV capsid variant described herein has the ability to enclose, e.g., encapsulate, a viral genome and/or is capable of entry into a cell, e.g., a mammalian cell. In some embodiments, the AAV capsid variant described herein may have modified tropism compared to that of a wild-type AAV capsid, e.g., the corresponding wild-type capsid.

Conserved: As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95%, about 98%, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of a polynucleotide or polypeptide or may apply to a portion, region or feature thereof.

Encapsulate: As used herein, the term “encapsulate” means to enclose, surround or encase. As an example, a capsid protein, e.g., an AAV capsid variant, often encapsulates a viral genome. In some embodiments, encapsulate within a capsid, e.g., an AAV capsid variant, encompasses 100% coverage by a capsid, as well as less than 100% coverage, e.g., 95% or less. For example, gaps or discontinuities may be present in the capsid so long as the viral genome is retained in the capsid, e.g., prior to entry into a cell.

Effective Amount: As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment of a therapeutic indication as compared to the response obtained without administration of the agent.

Epitope: As used herein, an “epitope” refers to a surface or region on one or more entities that is capable of interacting with an antibody or other binding biomolecule. For example, a protein epitope may contain one or more amino acids and/or post-translational modifications (e.g., phosphorylated residues) which interact with an antibody. In some embodiments, an epitope may be a “conformational epitope,” which refers to an epitope involving a specific three-dimensional arrangement of the entity(ies) having or forming the epitope. For example, conformational epitopes of proteins may include combinations of amino acids and/or post-translational modifications from folded, non-linear stretches of amino acid chains.

EvoMap™: As used herein, an EvoMap™ refers to a map of a polypeptide, wherein detailed informatics are presented about the effects of single amino acid mutations within the length of the polypeptide and their influence on the properties and characteristics of that polypeptide.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Fragment: A “fragment,” as used herein, refers to a portion. For example, an antibody fragment may comprise a CDR, or a heavy chain variable region, or a scFv, etc. In some embodiments, a fragment is a nucleic acid fragment.

Homology As used herein, the term “homology” refers to the overall relatedness between polymeric molecules, e.g. between polynucleotide molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the disclosure, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the disclosure, two protein sequences are considered to be homologous if the proteins are at least about 50%, 60%, 70%, 80%, or 90% identical for at least one stretch of at least about 20 amino acids.

Identity As used herein, the term “identity” refers to the overall relatedness between polymeric molecules, e.g., between polynucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology. Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H. and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)). In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

Isolated: As used herein, the term “isolated” refers to a substance or entity that is altered or removed from the natural state, e.g., altered or removed from at least some of the components with which it is associated in the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. Such polynucleotides could be part of a vector and/or such polynucleotides or polypeptides could be part of a composition, and still be isolated in that such vector or composition is not part of the environment in which it is found in nature. In some embodiments, an isolated nucleic acid is recombinant or may be incorporated into a vector.

Linker: As used herein “linker” refers to a molecule or group of molecules which connects two molecules. In some embodiments, linkers may be cleavable (e.g., through contact with an enzyme, change in pH, or change in temperature).

MicroRNA (miRNA or miR) binding site: As used herein, a “miR binding site” comprises a nucleic acid sequence (whether RNA or DNA, e.g., differ by “U” of RNA or “T” in DNA) that is capable of binding, or binds, in whole or in part to a microRNA (miR) through complete or partial hybridization. Typically, such binding occurs between the miR and the miR binding site in the reverse complement orientation. In some embodiments, the miR binding site is transcribed from the AAV vector genome encoding the miR binding site.

In some embodiments, a miR binding site may be encoded or transcribed in series. Such a “miR binding site series” or “miRBSs” may include two or more miR binding sites having the same or different nucleic acid sequence.

Modified: As used herein “modified” refers to a changed state or structure of a molecule. Molecules may be modified in many ways including chemically, structurally, and functionally.

Payload: As used herein, “payload” refers to any substance being delivered by an agent. For example, payloads may include therapeutic agents conjugated to antibodies for delivery to a cell, tissue, or region harboring an epitope of the antibody.

Polypeptide: As used herein, “polypeptide” means a polymer of amino acid residues (natural or unnatural) linked together most often by peptide bonds. The term, as used herein, refers to proteins, polypeptides, and peptides of any size, structure, or function. If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides and may be associated or linked. The term polypeptide may also apply to amino acid polymers in which one or more amino acid residues are an artificial chemical analogue of a corresponding naturally occurring amino acid.

Polypeptide variant: The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. In some embodiments, a variant comprises a sequence having at least about 50%, at least about 80%, or at least about 90%, identical (homologous) to a native or a reference sequence.

Peptide: As used herein, “peptide” is less than or equal to 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Preventing: As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Prophylactic: As used herein, “prophylactic” refers to a therapeutic or course of action used to prevent the spread of disease.

Prophylaxis: As used herein, a “prophylaxis” refers to a measure taken to maintain health and prevent the spread of disease.

Purified: As used herein, “purify,” “purified,” “purification” means to make substantially pure or clear from unwanted components, material defilement, admixture or imperfection. “Purified” refers to the state of being pure. “Purification” refers to the process of making pure.

Region: As used herein, the term “region” refers to a zone or general area. In some embodiments, when referring to a protein or protein module, a region may comprise a linear sequence of amino acids along the protein or protein module or may comprise a three-dimensional area, an epitope and/or a cluster of epitopes. In some embodiments, regions comprise terminal regions. As used herein, the term “terminal region” refers to regions located at the ends or termini of a given agent. When referring to proteins, terminal regions may comprise N- and/or C-termini

In some embodiments, when referring to a polynucleotide, a region may comprise a linear sequence of nucleic acids along the polynucleotide or may comprise a three-dimensional area, secondary structure, or tertiary structure. In some embodiments, regions comprise terminal regions. As used herein, the term “terminal region” refers to regions located at the ends or termini of a given agent. When referring to polynucleotides, terminal regions may comprise 5′ and/or 3′ termini.

RNA and DNA: As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

Sample: As used herein, the term “sample” refers to a portion or subset of larger entity. A sample from a biological organism or material is referred to herein as a “biological sample” and may include, but is not limited to, tissues, cells, and body fluids (e.g., blood, mucus, lymphatic fluid, synovial fluid, cerebrospinal fluid, saliva, amniotic fluid, amniotic cord blood, urine, vaginal fluid, and semen). Samples may further include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, and organs. Samples may further include a medium, such as a nutrient broth or gel, which may contain cellular components, such as proteins or nucleic acid molecules.

Spacer: As used here, a “spacer” is generally any selected nucleic acid sequence of, e.g., 1, 2, 3, 4, 5, 6, 7, 8, 9, or 10 nucleotides in length, which is located between two or more consecutive miR binding site sequences. Spacers may also be more than 10 nucleotides in length, e.g., 20, 30, 40, or 50 or more than 50 nucleotides.

Subject: As used herein, the term “subject” or “patient” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, neurodegenerative disease) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity or disfunction of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutic Agent: The term “therapeutic agent” refers to any agent that, when administered to a subject, has a therapeutic, diagnostic, and/or prophylactic effect and/or elicits a desired biological and/or pharmacological effect. Therapeutic agents capable of producing a biological effect in living organisms are referred to herein as “drugs.”

Therapeutically effective amount: As used herein, the term “therapeutically effective amount” means an amount of an agent (e.g., antibody or other therapeutic agent) to be delivered to a subject suffering from or susceptible to an infection, disease, disorder, and/or condition, that when delivered or administered in that amount is sufficient to treat, improve symptoms of, diagnose, prevent, and/or delay the onset of the infection, disease, disorder, and/or condition. In some embodiments, a therapeutically effective amount is provided in a single dose. In some embodiments, a therapeutically effective amount is administered in a dosage regimen that includes a plurality of doses. Those skilled in the art will appreciate that in some embodiments, a unit dosage form may be considered to include a therapeutically effective amount of a particular agent or entity if it includes an amount that is effective when administered as part of such a dosage regimen.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Conservative amino acid substitution: As used herein, a “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Variant: As used herein, the term “variant” refers to a polypeptide or polynucleotide that has an amino acid or a nucleotide sequence that is substantially identical, e.g., having at least 70%, 75%, 80%, 85%, 90%, 95%, or 99% sequence identity to a reference sequence. In some embodiments, the variant is a functional variant.

Functional Variant: As used herein, the term “functional variant” refers to a polypeptide variant or a polynucleotide variant that has at least one activity of the reference sequence.

Vector: As used herein, the term “vector” refers to any molecule or moiety which transports, transduces, or otherwise acts as a carrier of a heterologous molecule. In some embodiments, vectors may be plasmids. Vectors of the present disclosure may be produced recombinantly. The heterologous molecule may be a polynucleotide and/or a polypeptide.

Viral Genome: As used herein, the term “viral genome” refers to the nucleic acid sequence(s) encapsulated in an AAV particle. A viral genome comprises a nucleic acid sequence with at least one payload region encoding a payload and at least one ITR.

### EXAMPLES

The antibodies described herein, e.g., as provided in Table 4, were generated and characterized as described in Examples 1-11 in WO 2021/211753, which are hereby incorporated by reference in their entirety.

### Example 1. Tau Binding

Variable domain nucleic acid sequences from antibodies obtained from immunizations described in Examples 1 and 2 of WO 2021/211753 (the contents of which are hereby incorporated by reference in its entirety) were used to prepare recombinant mouse IgG antibodies. These candidate antibodies were analyzed for binding to ePHF and specificity for ePHF over wild type tau by direct ELISA.

For direct ePHF and wildtype tau ELISA, plates were first coated with ePHF or wild type tau. Antigen solutions were prepared in PBS and 50 μL were pipetted into each well. Plates were covered and incubated for one hour at 37° C. or overnight at 4° C. Plates were then washed and blocked by addition of 150 μl of blocking buffer to each well and incubated one hour at room temperature. Plates were then washed before addition of serially diluted candidate antibody samples prepared in blocking buffer. Detection of candidate antibody binding was carried out by washing plates and adding a solution of enzyme-labeled secondary antibody in blocking buffer to each well. Secondary antibody binding was detected by addition of substrate and spectrophotometric analysis of resulting reaction product. Half maximal effective concentration (EC50) for antibody binding to ePHF and wild type tau are presented in Table 20.

TABLE 20
ELISA results

| | ePHF EC50 | Wild type Tau |
|---|---|---|
| ID# | (nM) | EC50 (nM) |
| V0009 | 0.038 | No binding |
| V0004 | 0.083 | No binding |
| V0024 | 0.125 | No binding |
| V0022 | 0.070 | Not determined |
| V0023 | 0.110 | Not determined |
| V0052 | Weak binding | Not determined |

Results of a follow up analysis of antibodies V0022, V0009, V0023, V0024, and V0052 for selectivity for iPHF and ePHF over wild-type tau are shown in Table 27. Overall, the antibodies had a greater than 54-fold selectivity for iPHF over wild-type tau, with V0009, V0022, and V0052 having a greater than 390-fold selectivity for iPHF over wild-type tau. With regard to the selectivity for ePHF over wild-type tau, the anti-tau antibodies V0022, V0009, V0023, and V0024 had a greater than 120-fold selectivity for ePHF over wild-type tau, with V0009 and V0022 having a greater than 200-fold selectivity for ePHF over wild-type tau. Further, these antibodies demonstrated low polyspecificity and good stability in solution at 1 mg/mL.

TABLE 27
Selectivity Results

| Selectivity | Criteria | V0009 | V0022 | V0023 | V0024 | V0052 |
|---|---|---|---|---|---|---|
| iPHF: WT | ≥50 fold | >1450 | >838 | >54.5 | >80 | >396 |
| ePHF: WT | ≥50 fold | >415 | >222 | >140 | >123 | No |

### Example 2. Tissue Staining

Variable domain nucleic acid sequences from antibodies obtained from immunizations described above were used to prepare recombinant mouse IgG antibodies and tested for ability to bind pathological tau in human brain tissue sections. Cryo-preserved human brain tissue sections from patients with or without Alzheimer's disease (AD) were mounted on glass slides and washed with PBS. Endogenous peroxidase activity in tissue sections was quenched for 30 minutes at room temperature using a solution of 0.9% hydrogen peroxide and 0.02% Triton-X 100 in 1×PBS. Tissue sections were then washed with PBS and incubated for 1 hour at room temperature with a blocking solution of 10% normal goat serum in PBS with 0.02% Triton-X 100. Candidate antibody solutions were prepared by 1:500 dilution in PBS with protein diluent. Tissue sections were incubated in candidate antibody solutions for 1 hour at room temperature before washing in PBS to remove unbound antibody. Tissue sections were then treated with solutions of biotinylated goat-anti mouse IgG in PBS with protein diluent and incubated for 1 hour at room temperature. Tissue sections were again washed in PBS and treated with a solution of avidin-peroxidase conjugate before incubation for 30 minutes at room temperature. Tissue sections were again washed in PBS prior to treatment with a 3,3′ diaminobenzidine tetrahydrochloride (DAB) substrate solution to yield a brown enzymatic precipitate at sites of candidate antibody binding and peroxidase immunocomplex formation. Enzymatic reactions were allowed to proceed for about 1 minute before being halted by rinsing in PBS wash solution. Tissue sections were then mounted for microscopic evaluation of immunostaining (indicating candidate antibody binding). Immunostained tissue sections were ranked based on degree of staining observed. Results are shown in Table 24. In the Table, “−” indicates no staining, and “+,” “+/++,” “++,” “++/+++,” and “+++” indicate levels of positive staining from low to high. “+/−” indicates no staining, with the exception of some staining observed in non-disease tissue.

TABLE 24
Tissue staining results

| ID# | Staining level in AD tissue | Staining level in non-AD tissue |
|---|---|---|
| V0024 | +++ | − |
| V0009 | +++ | −/+ |
| V0004 | +++ | −/+ |
| V0022 | +++ | −/+ |
| V0023 | +++ | −/+ |
| V0052 | +/++ | + |

Multiple antibodies yielded positive staining in AD brain tissue sections with little or no positive staining observed in brain tissue sections from non-AD brain tissue.

Similar results were obtained using fixed human brain tissues when assessing antibodies V0004, V0009, V0022, V0023, V0024, and V0052 (Table 28). Similar results were also obtained for these antibodies when comparing brain tissue staining between wild type mice and FTD mutant tau transgenic mice carrying P301 S tau mutations.

TABLE 28
Evaluation of binding to tau pathology from patient AD and PSP cases

| Sample (IHC) | V0009 | V0022 | V0023 | V0024 | V0052 |
|---|---|---|---|---|---|
| IHC Fixed - Human AD Brain | Positive | Positive | Positive | Positive | Weak |
| IHC Fixed - Human Control (Ctl) Brain | Negative | Negative | Negative | Negative | Negative |
| IHC Fixed - Human PSP | Positive | Positive | Positive | Positive | Positive |
| | (weak) | | | | |
| IHC-Fixed - Human Ctl Brain | Negative | Negative | Negative | Negative | Negative |
| IHC Frozen - Human AD Brain | Positive | Positive | Positive | Positive | Positive |
| IHC Frozen - Human PSP | Positive | Positive | Positive | Positive | Positive |
| IHC Frozen - Human Ctl Brain | Negative | Negative | Negative | Negative | weak |

### Example 3. iPHF Affinity Analysis

Anti-human tau antibodies were assessed for affinity for iPHF by Octet (ForteBio, Menlo Park, CA) analysis. Recombinant mouse IgG antibodies were prepared with clone-specific variable domain pairs selected from those presented in Table 3 and mouse IgG1 constant domains. Candidate antibodies were immobilized on biosensor tips (ForteBio) in kinetic buffer (ForteBio). Biosensor tips were then washed before introduction of a solution of iPHF in kinetic buffer for analysis of association and dissociation with candidate antibodies. Affinity measurements (K_{D}) were obtained using Data Analysis HT version 11.1 and corrected for background and high-frequency noise. Results are presented in Table 25.

TABLE 25
Affinity analysis results by Octet

| | iPHF K_{D} | | IPHF K_{D} | | iPHF K_{D} |
|---|---|---|---|---|---|
| ID# | (nM) | ID# | (nM) | ID# | (nM) |
| V009 | 0.2 | V0023 | 4.0 | V0024 | 2.8 |
| V0022 | 0.3 | V0004 | 20.0 | V0052 | 0.6 |

The antibodies shown in Table 25 all demonstrated K_{D} values less than 150 nM, with antibodies V0009, V0022, V0052, V0024, and V0023 demonstrating K_{D} values less than 10 nM. Of the antibodies tested, all demonstrated weak or no affinity (greater than 200 nM K_{D}) for wild type tau, except for V00016, which demonstrated nearly equivalent affinity for wild type tau.

Antibodies V0022, V009, V0023, V0024, and V0052 were also assessed for binding to iPHF tau by surface plasmon resonance on a Biacore 8K instrument. Affinity (K_{D}), including kₒₙ and k_{off}, for each of the anti-tau antibodies tested are provided in Table 28. The data obtained by SPR correlated with what was observed via Octet. These data demonstrated that the antibodies tested bind to immunopurified PHF tau with high affinity and none of the antibodies demonstrated any binding to wild-type tau.

TABLE 28
Affinity analysis results by Biacore/SPR analysis

| ID# | KD (pM) | ka (1/Ms) | kd (1/s) |
|---|---|---|---|
| V0022 | 43.9 | 1.70E+06 | 7.09E−05 |
| V0009 | 296.5 | 2.92E+06 | 7.87E−04 |
| V0023 | 57.8 | 5.22E+05 | 3.00E−05 |
| V0024 | 124.5 | 5.89E+05 | 7.32E−05 |
| V0052 | 4465 (KD1), | 3.0E+07, | 1.3E−01, |
| | 2640 (KD2) | 6.6E+04 | 1.8E−04 |

### Example 4. Fine Epitope Mapping of V0022, V0023, and V0024 Anti-Tau Antibodies

As described in Example 9 of WO 2021/211753 (which is hereby incorporated by reference in its entirety), V0022, V0023, and V0024 antibodies were demonstrated to bind close to the tau C-terminus with affinity for peptides corresponding with residues 409-436 of human tau (SEQ ID NO: 920). These antibodies demonstrated the highest affinity (V0022 K_{D}=3.06×10⁻¹⁰ M; V0023 K_{D}=2.07×10⁻¹⁰ M; V0024 K_{D}=2.25×10⁻¹⁰ M) for peptides corresponding with residues 413-430 of human tau (SEQ ID NO: 920). This Example describes the further characterization of the binding specificity of three anti-tau antibodies.

To assess the binding characteristics of V0022, V0023, and V0024, the ability of these antibodies to bind to different phosphorylated species of the Tau409-426 peptide, specifically pS409, pS412, pS413, pT414, pS416, pS422, or a combination thereof (Table 29), was tested by a one point ELISA using a high resolution sub-phospho-peptide library. Briefly, peptides were generated that showed all possible combinations of phosphorylation patterns within known epitopes containing multiple possible phosphorylation sites. One-point ELISA was then used to determine differential binding based on the individual phosphorylation patterns of each peptide. OD values (450 nm) were collected, where a stronger positive signal is shown as a higher OD value, which is indicative of increased binding. A kinetic ELISA was also performed and EC50 values were calculated for each the affinity of each antibody tested to each peptide in Table 29.

TABLE 29
Tau409-426 phospho-peptides

| SEQ | | |
|---|---|---|
| ID | Description | Sequence |
| 32 | pS409, pS412, pS413, pT414, | GSGS(pS)NV(pS)(pS)(pT)G(pS)IDLVD(pS)PQLA |
| | pS416, pS422 | |
| 33 | pS422 | GSGSSNVSSTGSIDLVD(pS)PQLA |
| 34 | pS416, pS422 | GSGSSNVSSTG(pS)IDLVD(pS)PQLA |

The V0022, V0023, and V0024 all demonstrated the same binding pattern and were able to bind to all phospho-tau peptides tested, including the peptide that was phosphorylated at pS409, pS412, pS413, pT414, pS416, pS422, the peptide phosphorylated at pS422, and the peptide phosphorylated at pS416 and pS422 (Table 30). This demonstrated that pS422 was necessary and sufficient for binding of the V0022, V0023, and V0024 to phospho-tau. Kinetic ELISA confirmed that V0022, V0023, and V0024 bind pS422 specifically.

TABLE 30
EC50 for V0022, V0023, and V0024 against
different phosphorylated tau peptides

| | EC50 (nM) | | |
|---|---|---|---|
| | pS409, pS412, pS413, pT414, | | |
| Antibody | pS416, pS422 | pS422 | pS416, pS422 |
| V0022 | 0.3023 | 0.2876 | 0.2826 |
| V0023 | 0.217 | 0.2337 | 0.2522 |
| V0024 | 0.2223 | 0.2333 | 0.2201 |

### Example 5. Engineering Viral Genomes for the Expression of Anti-Tau Antibodies

A viral genomes was designed for AAV delivery of the anti-tau antibody V0022 (Table 4). The nucleotide sequence from 5′ ITR to 3′ ITR is provided herein in Tables 18 and 19, as SEQ ID NO: 15.

The viral genome construct comprises a nucleotide sequence encoding an antibody that binds to tau. The nucleotide sequence was designed to encode an antibody heavy chain signal sequence (SEQ ID NO: 1 (amino acid) and SEQ ID NO: 1083 (DNA)); a nucleotide sequence encoding a heavy chain variable region (SEQ ID NO: 21 (amino acid) and SEQ ID NO: 7 (DNA); a nucleotide sequence encoding a heavy chain constant region (SEQ ID NO: 16 (amino acid) and SEQ ID NO: 805 (DNA)); a nucleotide sequence encoding a first linker (SEQ ID NO: 1724); a nucleotide sequence encoding a second linker (SEQ ID NO: 1726); a nucleotide sequence encoding a light chain signal sequence (SEQ ID NO: 2 (amino acid) and SEQ ID NO: 1084 (DNA)); a nucleotide sequence encoding a light chain variable region (SEQ ID NO: 93 (amino acid) and SEQ ID NO: 11 (DNA)); and a nucleotide sequence encoding a light chain constant region (SEQ ID NO: 18 (amino acid) and SEQ ID NO: 17 (DNA)). The nucleotide sequence encoding the anti-tau antibody was operably linked to a CMVie enhance (SEQ ID NO: 1050) and a CBA promoter variant (SEQ ID NO: 1042).

The viral genome construct also comprised a 5′ ITR of SEQ ID NO: 1035, an intron of SEQ ID NO: 1068, a polyA signal region of SEQ ID NO:1134, and a 3′ ITR of SEQ ID NO: 1037.

### Example 6. Inhibition of Tau Pathology by Anti-Tau Antibodies in Hippocampal P301S Seeding Model

In this Example, the AD-PHF seeded hippocampal P301S model was used for in vivo efficacy studies of anti-tau antibodies and vectorized forms thereof. To establish the hippocampal seeding model, AD brain-derived PHFs was injected into the left hippocampus of 8-weeks-old P301S mice. Efficacy of the V0022, V0009, V0023, V0024, V0052 antibodies alone (passive immunization) and the vectorized V0022 antibody were then investigated.

Briefly, for the passive immunization, the V0022, V009, V0023, V0024, and V0052 antibodies were administered intraperitoneally at 40 mg/kg two times in the week prior to the hippocampal seeding of the PHFs (2 doses), and five additional doses (40 mg/kg per dose) were administered intraperitoneally each week after seeding. For the vectorized administration, AAV particles comprising a VOY101 capsid protein (SEQ ID NO: 803), and a viral genome of SEQ ID NO: 15 (see, e.g., Example 5), which encoded the V0022 antibody under the control of a CMVie enhancer/CBA promoter variant (VOY101_V0022) were administered by intravenous injection two weeks prior to seeding at a dose of either 1e13 vg/kg or 3e13 vg/kg. An IgG or a PBS vehicle was administered as a control. At 6 weeks post-seeding and 8-weeks post the initial dose of either the antibodies alone or the vectorized anti-tau antibody, the brains and hippocampi of each animal were isolated for AT8 ELISA to assess tau pathology.

First, the ability of injected PHFs to induce tau pathology was determined in P301 S mice without the injection of anti-tau antibodies. A significant increase of AT8 immunoreactivity (IR) was detected in the PHF injected ipsilateral site but not in vehicle (PBS) injected site. In the tau seed injected mice, tau pathology was detected in the contralateral site to a lesser extent, indicating tau pathology induced by injected PHF can spread across hippocampus. Tau pathology was also detected by IHC staining with AT100 anti-tau antibody. A significant number of CA neurons on the ipsilateral site exhibited tau pathology (AT100 positives).

Table 31 provides the reduction in AT8 IR pathology in the mice following passive administration of the V0009, V0022, V0023, V0024, and V0052 antibodies normalized to the PBS vehicle control. As shown in Table 31, a significant reduction of tau pathology was observed in both the ipsilateral and contralateral hippocampus in mice passively treated with V0022, V0023, and V0024. Additionally, all antibodies investigated demonstrated a significant reduction in tau pathology in the contralateral hippocampus. Comparing the AT8 IR in the ipsilateral hippocampus versus the contralateral hippocampus also demonstrated a robust reduction of tau pathology spreading across the hippocampus in the mice treated by passive immunization of the V0022, V0023, and V0024 anti-tau antibodies.

TABLE 31
Efficacy of anti-tau antibodies administered by
passive in vivo compared to PBS vehicle control

| | Reduction of AT8 IR vs vehicle (PBS) | |
|---|---|---|
| Anti- | Ipsilateral | Contralateral |
| body | Efficacy | Efficacy |
| V0009 | None | 43% |
| V0022 | 74% | 71% |
| V0023 | 52% | 55% |
| V0024 | 67% | 72% |
| V0052 | none | 55% |

Table 32 provides the reduction in AT8 IR pathology normalized to an IgG control (efficacy) as well as the viral genome and/or antibody levels in the hippocampus and cerebral spinal fluid (CSF) (biodistribution) in the mice following passive administration (V0022) or vectorized administration (VOY101_V0022) of the V0022 antibody. As shown in Table 32, a dose dependent increase in viral genome or antibody levels was observed in the central nervous system and CSF following vectorized delivery of the V0022 antibody. Additionally, significant reduction in tau pathology was observed in mice treated with the high and low doses of the vectorized V0022 antibody as well as those treated with passive delivery of the V0022 antibody. In fact, passive administration of V0022 resulted in comparable levels of reduction in tau pathology when compared to vectorized delivery at both the high and low dose. These data demonstrate that V0022 administered passively or in a vectorized form (e.g., vectorized in an AAV particle) can significantly reduce tau pathology.

TABLE 32
Efficacy and biodistribution following passive and
vectorized administration of the V0022 antibody

| | | | | | | Reduction of |
|---|---|---|---|---|---|---|
| | | | Viral | Hippocampus | CSF Ab | AT8 pathology |
| Antibody | | | genomes | Ab level | levels | normalized to |
| (Ab) | Dose | | per cell | (ng/g) | (ng/mL) | IgG control |
| VOY101_V0022 | 1e13 | vg/kg | 8 vg/cell | 2,878 | 358 | 71% |
| (vectorized) | 3e13 | vg/kg | 25 vg/cell | 7,310 | 728 | 78% |
| V0022 | 40 | mg/kg | N/A | 887 | 194 | 68.4% |
| (passive) | | | | | | |

### Example 7. Inhibition of Tau Pathology by the V0022 Antibody in a P301S Intrinsic Model

In this Example, the P301 S intrinsic model was used for in vivo efficacy studies of the V0022 anti-tau antibody and a vectorized form thereof.

Eight-week-old P301 S mice were dosed with AAV particles comprising a VOY101 capsid protein (SEQ ID NO: 803), and a viral genome of SEQ ID NO: 15 (see, e.g., Example 5), which encoded the V0022 antibody under the control of a CMVie enhancer/CBA promoter variant (VOY101_V0022) by intravenous administration or the V0022 antibody by intraperitoneal injection, weekly for 13 weeks at a dose of either 1e13 vg/kg or 3e13 vg/kg for VOY101_V0022 or a dose of 40 mg/kg for the V0022 antibody. Control mice were administered with an IgG control. Tau pathology was not yet developed in these mice at 8 weeks but in untreated mice, continues to progress throughout their lifespan. At 20 weeks of age (e.g., 12 weeks post initial dose of VOY101_V0022 or V0022), mice were sacrificed and the hippocampus and cortex were isolated for biochemical analyses.

Table 33 provides the reduction in AT8 IR pathology normalized to an IgG control (efficacy) as well as the viral genome and/or antibody levels in the hippocampus and CSF (biodistribution) in the mice following passive administration (V0022) or vectorized administration (VOY101_V0022) of the V0022 antibody. As shown in Table 33, a dose dependent increase in viral genome was observed in the central nervous system regions and CSF following vectorized delivery of the V0022 antibody. V0022 antibody expression was observed in the CNS regions as well as the CSF in mice treated by both the vectorized or passive delivery of the V0022 antibody. Additionally, a reduction in tau pathology was observed in mice treated with the high and low doses of the vectorized V0022 antibody as well as those treated with passive delivery of the V0022 antibody. In fact, passive administration of V0022 resulted in comparable levels of reduction in tau pathology when compared to vectorized delivery at both the high and low dose. These data demonstrate that V0022 administered passively or in a vectorized form (e.g., vectorized in an AAV particle) can significant reduce tau pathology.

TABLE 33
Efficacy and biodistribution in the cortex and hippocampus following
passive and vectorized administration of the V0022 antibody

| | | | | | | Reduction of AT8 | |
|---|---|---|---|---|---|---|---|
| | Viral genomes | | Antibody Levels | | | pathology normalized | |
| Antibody and | per cell | | Cortex | Hippocampus | CSF | to IgG control | |
| Dose | Cortex | Hippocampus | (ng/g) | (ng/g) | (ng/mL) | Cortex | Hippocampus |
| VOY101_V0022 | 10.3 | 4.7 | 2,970 | 3405 | 330 | 42% | 53% |
| 1e13 vg/kg | | | | | | | |
| VOY101_V0022 | 19.2 | 8.9 | 5,451 | 6277 | 564 | 61% | 66% |
| 3e13 vg/kg | | | | | | | |
| V0022 40 mg/kg | N/A | N/A | 98 | 628 | 396 | 30% | 40% |

In a second experiment, female 12-week old P301S mice were dosed the V0022 antibody or a vehicle control by intraperitoneal injection weekly for 5 weeks (day 1, 8, 15, 22, and 29) at a dose of 80 mg/kg. At 16-weeks post-initial dose, mice were sacrificed and the cortex, serum, and CSF were isolated for biochemical analyses.

Table 39 provides the pathological tau levels in the cortex and the antibody levels in the CSF and serum. A 66% reduction in in pathological tau signal was observed in the mice that received the V0022 antibody compared to the vehicle control by ELISA which was used to quantify the unbound and bound pathological tau. This demonstrated target engagement by the V0022 antibody.

The tolerability of V0022 was also tested in the P301S mouse following 5 weekly intravenous doses of 30 mg/kg, 80 mg/kg, or 120 mg/kg and all doses were well tolerated. The serum pharmacokinetics (PK) of 80 mg/kg of V0022 in the P301S mouse exhibited a profile expected from a mouse IgG1 antibody, and had a half-life of approximately 12.6 days. Model based PK parameter estimates yielded a clearance rate (C_{L}) of 0.166 mL/day, and central and peripheral volumes of distribution were 0.839 mL and 1.87 mL, respectively.

TABLE 39
Efficacy and biodistribution in the cortex and hippocampus following
passive and vectorized administration of the V0022 antibody

| | Pathological Tau Levels | | | |
|---|---|---|---|---|
| | Cortex | Cortex (ng/mg) ROUT | Antibody Levels | |
| Antibody and | (ng/mg) | Identified | CSF | Serum |
| Dose | All Samples | Outliers Removed | (ng/mL) | (ng/mL) |
| V0022 | 48 | 29.9 | 818.4 | 1081 |
| 80 mg/kg | | | | |
| Vehicle | 108 | 89.0 | 0 | 0 |

### Example 8. Evaluation and Characterization of Humanized V0022 Antibodies

This example describes the humanization of the V0022 antibody and evaluation and characterization of said humanized antibodies. The V0022 antibody specifically targets an epitope in the C-terminus of tau.

### A. Generation of Humanized V0022 Antibodies

The HC CDRs (HC CDR1 of SEQ ID NO: 64, HC CDR2 of SEQ ID NO: 1145, and HC CDR3 of SEQ ID NO: 1167) from the murine heavy chain variable region (SEQ ID NO: 21) of V0022, and the LC CDRs (LC CDR1 of SEQ ID NO: 1146, LC CDR2 of SEQ ID NO: 529, and LC CDR3 of SEQ ID NO: 571) of the light chain variable region (SEQ ID NO: 93) of V0022 generated in Example 1 of WO 2021/211753 (the contents of which are hereby incorporated by reference in its entirety), were grafted onto human framework regions. Five different humanized VH sequences and five different humanized VL sequences were generated, and are provided in Tables 4A and 4B, respectively. Each of the five humanized heavy chains comprised a human IgG4 isotype constant region which comprised an S to P mutation at position 228, numbered according to EU numbering. Each of the five humanized light chains comprised a human IgK constant domain (allotype Km3). The humanized V0022 antibody sequences are provided in Tables 4A, 4B, and 4C. The VH/VL and heavy and light chain pairings for the humanized antibodies investigated in this Example are shown in Table 34.

TABLE 34
Humanized V0022 Antibodies

| Antibody | Sequences | Antibody | Sequences |
|---|---|---|---|
| hV0022 | VH: SEQ ID NO: 21 | Ab3 | VH: SEQ ID NO: 70 |
| control | VL: SEQ ID NO: 93 | | VL: SEQ ID NO: 72 |
| | HC: SEQ ID NO: 65 | | HC: SEQ ID NO: 173 |
| | LC: SEQ ID NO: 66 | | LC: SEQ ID NO: 175 |
| Ab2 | VH: SEQ ID NO: 67 | Ab9 | VH: SEQ ID NO: 70 |
| | VL: SEQ ID NO: 72 | | VL: SEQ ID NO: 73 |
| | HC: SEQ ID NO: 170 | | HC: SEQ ID NO: 173 |
| | LC: SEQ ID NO: 175 | | LC: SEQ ID NO: 176 |
| Ab6 | VH: SEQ ID NO: 67 | Ab10 | VH: SEQ ID NO: 70 |
| | VL: SEQ ID NO: 73 | | VL: SEQ ID NO: 74 |
| | HC: SEQ ID NO: 170 | | HC: SEQ ID NO: 173 |
| | LC: SEQ ID NO: 176 | | LC: SEQ ID NO: 177 |
| Ab7 | VH: SEQ ID NO: 68 | Ab11 | VH: SEQ ID NO: 71 |
| | VL: SEQ ID NO: 72 | | VL: SEQ ID NO: 72 |
| | HC: SEQ ID NO: 171 | | HC: SEQ ID NO: 174 |
| | LC: SEQ ID NO: 175 | | LC: SEQ ID NO: 175 |
| Ab8 | VH: SEQ ID NO: 69 | Ab4 | VH: SEQ ID NO: 71 |
| | VL: SEQ ID NO: 72 | | VL: SEQ ID NO: 73 |
| | HC: SEQ ID NO: 172 | | HC: SEQ ID NO: 174 |
| | LC: SEQ ID NO: 175 | | LC: SEQ ID NO: 176 |
| Ab1 | VH: SEQ ID NO: 69 | Ab5 | VH: SEQ ID NO: 71 |
| | VL: SEQ ID NO: 73 | | VL: SEQ ID NO: 74 |
| | HC: SEQ ID NO: 172 | | HC: SEQ ID NO: 174 |
| | LC: SEQ ID NO: 176 | | LC: SEQ ID NO: 177 |

B. Affinity. Selectivity. And Immunogenicity of Humanized V0022 Antibodies

Different pairings of the humanized heavy and light chains of the V0022 antibody provided in Tables 4A and 4B, respectively, were analyzed to determine their affinity for immuno-purified PHF (iPHF) and a phosphorylated tau peptide of SEQ ID NO: 33 (pTau; phosphorylated at position S422 numbered according to wild-type tau of SEQ ID NO: 920), as well as their selectivity in detecting enriched PHF (ePHF) compared to wild-type Tau. The humanized variants were compared to the murine V0022 antibody control, which comprised a VH of SEQ ID NO: 21, a heavy chain of SEQ ID NO: 65, a VL of SEQ ID NO: 93, and a light chain of SEQ ID NO: 66 (see, e.g., Table 4 above). The phosphorylated tau peptide of SEQ ID NO: 33 comprising a phosphorylated serine at position 422, numbered according to SEQ ID NO: 920, was investigated as this includes the epitope for the humanized V0022 antibodies (see, e.g., Example 4).

Binding of the various humanized antibodies to iPHF or the phosphorylated tau peptide of SEQ ID NO: 33 was assessed by SPR on a Biacore 8K instrument. iPHF or the phosphorylated tau peptide was directly immobilized on the chip. iPHF was directly immobilized on CM5 sensor chip by amine coupling at 199 RU density. For the phosphorylated tau peptide, 1 μg/ml of the biotinylated phosphorylated peptide was captured on Biotin CAP chip via CAPture reagent to achieve 5-10 RU levels. Antibody was then injected using Single Cycle Kinetics (SCK) mode with association and dissociation times of 5 and 10 min, respectively, at a concentration ranging from 0.78 to 2.5 nM. The affinity value, or rate of dissociation (K_{D}), was subsequently calculated. As shown in Table 35, the humanized V0022 antibodies tested all bound to immunopurified PHF tau and the phosphorylated tau peptide with high affinity, as evidenced by the K_{D} values in the picomolar range.

The ability of the various humanized antibodies to bind ePHF and their specificity for ePHF compared to wild-type tau was also investigated by ELISA. The various humanized antibodies were exposed to a plate coated with either ePHF or wild-type tau. OD readings were taken at 450 nm and plotted against the antibody concentrations tested, and the half maximal effective concentration (EC50) for antibody binding to ePHF or wild type tau was calculated (Table 35). As provided in Table 35, all antibodies showed high affinity for ePHF but no binding to wild-type tau, indicating strong and selective binding to ePHF.

TABLE 35
Affinity and selectivity of humanized V0022 antibodies

| | | Affinity to | | | |
|---|---|---|---|---|---|
| | | pS422 tau | | | |
| | Affinity to | peptide (SEQ | Affinity to | Selectivity: | Inhibition of |
| | iPHF | ID NO: 33) | ePHF EC50 | ePHF:WT | ePHF seeding in |
| Antibody | (pM) | (pM) | (nM) | rec. Tau | Biosensor Cells |
| V0022 control | 16.5 | | | | 18.2 |
| VH: SEQ ID NO: 21 | | | | | |
| VL: SEQ ID NO: 93 | | | | | |
| HC: SEQ ID NO: 8 | | | | | |
| LC: SEQ ID NO: 12 | | | | | |
| hV0022 control | 16.2 | | 0.08 | >240 | |
| Ab2 | 29.3 | 38.6 | 0.15 | >133 | 78.1 |
| Ab6 | 39.1 | 39.8 | 0.16 | >124.8 | 17.6 |
| Ab7 | 33.4 | 39.8 | 0.13 | >149.9 | 49.8 |
| Ab8 | 49.6 | 53.3 | 0.17 | >119 | 17.6 |
| Ab1 | 43.4 | 40.8 | 0.085 | >232.9 | 32.2 |
| Ab3 | 32.5 | 45.7 | 0.095 | >210.3 | 58.1 |
| Ab9 | 44 | 76.3 | 0.1 | >198.4 | 49.8 |
| Ab10 | 30.4 | 43.6 | 0.14 | >118.8 | 34.5 |
| Ab11 | 24.9 | 40.3 | 0.16 | >113.8 | 24.1 |
| Ab4 | 35.3 | 36.1 | 0.18 | >122 | 37.9 |
| Ab5 | 24.8 | 29.4 | 0.19 | >128.5 | 43.8 |

These humanized V0022 antibodies also demonstrated low polyspecificity in a baculovirus particles (BVP) ELISA (Table 36), which measures binding to empty BVPs. Predicted aggregation and post-translational modification (PTM) liability were also assessed by in-silico assays and the values are provided for each humanized antibody tested in Table 36.

TABLE 36
Additional characteristics of humanized V0022 antibodies

| Antibody | Polyspecificity | PTM liability | Predicted aggregation |
|---|---|---|---|
| hV0022 control | 3.9 | | Inconclusive |
| Ab2 | 1.8 | 0.505 | Low |
| Ab6 | 1.6 | 0.505 | Inconclusive |
| Ab7 | 6 | 0.505 | Inconclusive |
| Ab8 | 1.6 | 0.505 | Inconclusive |
| Ab1 | 1.7 | 0.505 | Inconclusive |
| Ab3 | 2.2 | 0.505 | Inconclusive |
| Ab9 | 1.8 | 0.505 | Inconclusive |
| Ab10 | 3.2 | 0.505 | Low |
| Ab11 | 1.6 | 0.505 | Low |
| Ab4 | 1.4 | 0.505 | Low |
| Ab5 | 2.7 | 0.505 | Low |

The humanized antibodies were also assessed for their ability to bind pathological tau in human brain tissue sections isolated from the cortex of patients with or without Alzheimer's disease (AD). Staining was performed as described in Example 2. As provided in Table 37, all antibodies tested demonstrated specific binding to neuronal tau pathology with minimal staining of non-AD cortex tissues. A desirable PTM liability score for the humanized antibodies ranges from 0-1.

TABLE 37
Evaluation of binding to human AD tau
pathology of humanized V0022 antibodies

| | | IHC Fixed | | Western |
|---|---|---|---|---|
| | IHC Fixed | Human | Western | Frozen Human |
| | Human | Control | Frozen Human | Control |
| Antibody | AD Brain | Brain | AD Brain | Brain |
| V0022 control | Positive | Negative | Positive | Negative |
| VH: SEQ ID NO: 21 | | | | |
| VL: SEQ ID NO: 93 | | | | |
| HC: SEQ ID NO: 8 | | | | |
| LC: SEQ ID NO: 12 | | | | |
| hV0022 control | Positive | Negative | Positive | Negative |
| Ab2 | Positive | Negative | Positive | Negative |
| Ab6 | Positive | Negative | Positive | Negative |
| Ab7 | Positive | Negative | Positive | Negative |
| Ab8 | Positive | Negative | Positive | Negative |
| Ab1 | Positive | Negative | Positive | Negative |
| Ab3 | Positive | Negative | Positive | Negative |
| Ab9 | Positive | Negative | Positive | Negative |
| Ab10 | Positive | Negative | Positive | Negative |
| HC5LC1 | Positive | Negative | Positive | Negative |
| Ab4 | Positive | Negative | Positive | Negative |
| Ab5 | Positive | Negative | Positive | Negative |

The productivity for antibodies Ab1, Ab2, Ab3, Ab4, and Ab5 was also measured as yield from a 2L stable pool of mammalian cells (Table 40). As shown in Table 40, Ab1, Ab2, and Ab3 led to higher productivity and yield, as compared to Ab4 and Ab5.

TABLE 40
Productivity of humanized V0022 antibodies

| | Ab2 | Ab1 | Ab3 | Ab4 | Ab5 |
|---|---|---|---|---|---|
| Productivity - yield from | 355 mg | 456 mg | 666 mg | 151 mg | 121 mg |
| 2 L stable pool of | | | | | |
| mammalian cells | | | | | |

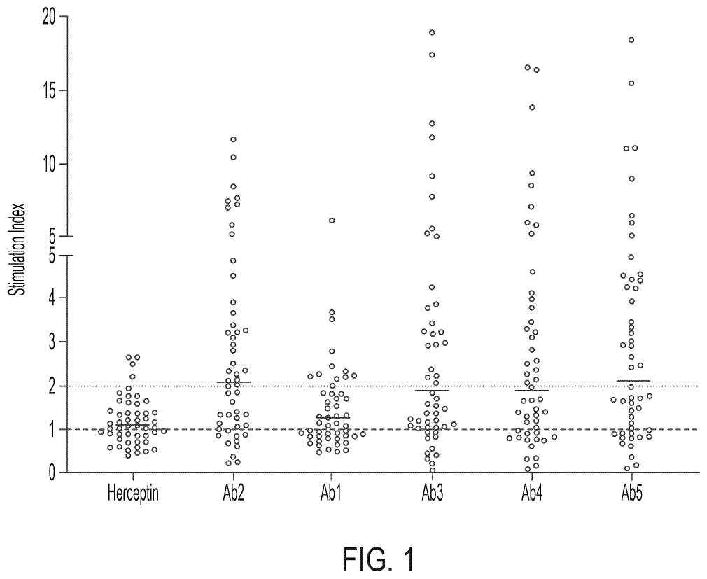

The relative immunogenicity risk of the humanized antibodies, Ab1-Ab5, were assessed by a CD4+ T cell proliferation assay. Samples of the antibodies were incubated with PBMC cells from 50 healthy donors that were representative for the global population based on HLA-DRB1 expression, and cell proliferation was measured by flow cytometry. The stimulation index (SI) was calculated for each antibody as the ratio of the number of proliferating T cells in the sample over the blank (FIG. 1). An SI index of 2.0 or greater was considered a positive response. Ab1 demonstrated a mean SI of less than 2.0, whereas the other antibodies tested (Ab2, Ab3, Ab4, and Ab5) demonstrated SI values closer to 2.0 or greater. The magnitude of the difference between the SI of Ab1 relative to the other humanized antibodies tested was statistically significant (p-value was less than or equal to 0.05). The percentage of responding donors was also calculated (Table 38). Herceptin was used as a control antibody as it has been shown to have a low risk of immunogenicity and less than 10% responders. As shown in Table 38, Ab1 demonstrated the lowest immunogenicity risk, as well as the lowest number of responding donor relative to the other humanized antibodies tested.

TABLE 38
Relative immunogenicity risk of humanized V0022 antibodies

| | # of Responding donors | % of Responding |
|---|---|---|
| Antigen | of 50 total donors | donors |
| KLH (control) | 50 | 100 |
| Herceptin (control) | 4 | 8 |
| Ab2 | 27 | 54 |
| Ab1 | 12 | 24 |
| Ab3 | 23 | 46 |
| Ab4 | 24 | 48 |
| Ab5 | 25 | 50 |

Antibody Ab1 was administered to P301 S mice (intrinsic model) intravenously at a dose of 120 mg/kg on day 1, 8, 15, 22, and 29. Serum was collected on days 6, 13, 20, 27 and 30. Mice administered Ab1 tolerated all 5 doses with no body weight changes and they showed no change in body temperature for up to 60 minutes after receiving the antibody doses. Ab1 antibody levels were assessed in the serum using a pS422 peptide ELISA. The mice showed no decreased serum antibody concentrations over 30 days from the initial dose. Ab1 levels were approximately 500 μg/mL for at least 6-27 days post initial dose and increased to up to about 1000 μg/mL by day 30.

The tolerability and pharmacokinetics of Ab1 was also investigated in non-human primates (NHPs) (cynomolgus macaques, Macaca fascicularis). The NHPs were administered a single intravenous dose of the antibody. The NHP was either given a high dose or a mid level dose of the Ab1. Both doses were well tolerated in the NHPs and no adverse effects were observed.

Taken together these data demonstrate that the humanized V0022 antibodies generated are capable of selective binding with strong affinity to pathological tau, e.g., iPHF and ePHF, but not wild-type tau (demonstrated selectivity). These humanized antibodies were also able to bind pathological tau in in brain samples from patients with Alzheimer's disease. Additionally, Ab1 was well tolerated in a mouse model of a tauopathy (e.g., the P301 S intrinsic model), demonstrated higher productivity and reduced immunogenicity, blocked paired helical filaments seed-induced tau aggregates in vitro, and selectively stained tau tangles in AD brain samples and the P301 S mouse. These data support use of Ab1 in patients with, e.g., mild dementia or mild cognitive impairment due to Alzheimer's disease (AD).

### VIII. Equivalents and Scope

The disclosures of the cited sources, for example references, publications, databases, and database entries, cited herein, are incorporated into this application by reference, even if not expressly stated in the citation.

While this invention has been disclosed with reference to specific aspects and embodiments, it is apparent that other aspects, embodiments, and variations of this invention may be devised by others skilled in the art without departing from the true spirit and scope of the invention. The appended claims are intended to be construed to include all such aspects and equivalent variations.

1.-54. (canceled)

55. An antibody that binds to human tau, which comprises a heavy chain variable region (VH) comprising a heavy chain complementarity determining region 1 (HC CDR1) comprising the amino acid sequence of SEQ ID NO: 1144, a HC CDR2 comprising the amino acid sequence of SEQ ID NO: 1145, and a HC CDR3 comprising the amino acid sequence of SEQ ID NO: 410; and a light chain variable region (VL) comprising a light chain complementarity determining region 1 (LC CDR1) comprising the amino acid sequence of SEQ ID NO: 1146, a LC CDR2 comprising the amino acid sequence of SEQ ID NO: 529, and a LC CDR3 comprising the amino acid sequence of SEQ ID NO: 571; and wherein:
(i) the VH comprises the amino acid E at position 1; V at position 5; S at position 7; A at position 9; V at position 11; K at position 12; K at position 19; V at position 20; R at position 67; V at position 68; M at position 70; I at position 76; A at position 79; Y at position 80; M at position 81; E at position 82; R at position 85; D at position 89, L at position 113; and S at position 115, numbered according to SEQ ID NO: 21; and the VL comprises the amino acid S at position 7; T at position 14; Q at position 17; P at position 18; Q at position 42; R at position 44; R at position 50; S at position 68; V at position 88; Y at position 92; and Q at position 105, numbered according to SEQ ID NO: 93;
(ii) the VH comprises the amino acid V at position 5; E at position 6; S at position 7; A at position 9; V at position 11; K at position 12; A at position 16; K at position 19; V at position 20; M at position 48; R at position 67; V at position 68; I at position 70; A at position 76; S at position 77; A at position 79; Y at position 80; M at position 81; E at position 82; R at position 87; T at position 91; and T at position 113, numbered according to SEQ ID NO: 21; and the VL comprises the amino acid S at position 7; T at position 14; Q at position 17; P at position 18; Q at position 42; R at position 44; R at position 50; S at position 68; V at position 88; Y at position 92; and Q at position 105, numbered according to SEQ ID NO: 93;
(iii) the VH comprises the amino acid V at position 5; S at position 7; A at position 9; V at position 11; K at position 12; A at position 16; K at position 19; V at position 20; Q at position 43; M at position 48; R at position 67; V at position 68; M at position 70; T at position 76; S at position 77; R at position 87; and T at position 91, numbered according to SEQ ID NO: 21; and the VL comprises the amino acid I at position 2; S at position 7; S at position 12; T at position 14; P at position 15; Q at position 17; P at position 18; Q at position 50; S at position 68; V at position 88; Y at position 92; Q at position 105; and V at position 109, numbered according to SEQ ID NO: 93; or
(iv) the VH comprises the amino acid V at position 5; S at position 7; A at position 9; V at position 11; K at position 12; A at position 16; K at position 19; V at position 20; Q at position 43; M at position 48; R at position 67; V at position 68; M at position 70; T at position 76; S at position 77; R at position 87; and T at position 91, numbered according to SEQ ID NO: 21; and the VL comprises the amino acid I at position 2; S at position 11; T at position 14; Q at position 17; P at position 18; Q at position 42; R at position 44; P at position 48; R at position 50; S at position 68; A at position 72; N at position 81; V at position 88; and Q at position 105, numbered according to SEQ ID NO: 93.

56. The antibody of claim 55, wherein:
(i) the VH comprises an amino acid sequence at least 95% identical to the amino acid sequence of SEQ ID NO: 70; and the VL comprises an amino acid sequence at least 95% identical to the amino acid sequence of SEQ ID NO: 72;
(ii) the VH comprises an amino acid sequence at least 95% identical to the amino acid sequence of SEQ ID NO: 67; and the VL comprises an amino acid sequence at least 95% identical to the amino acid sequence of SEQ ID NO: 72;
(iii) the VH comprises an amino acid sequence at least 95% identical to the amino acid sequence of SEQ ID NO: 71; and the VL comprises an amino acid sequence at least 95% identical to the amino acid sequence of SEQ ID NO: 73; or
(iv) the VH comprises an amino acid sequence at least 95% identical to the amino acid sequence of SEQ ID NO: 71; and the VL comprises an amino acid sequence at least 95% identical to the amino acid sequence of SEQ ID NO: 74.

57. The antibody of claim 55, wherein:
(i) the VH comprises an amino acid sequence at least 98% identical to the amino acid sequence of SEQ ID NO: 70; and the VL comprises an amino acid sequence at least 98% identical to the amino acid sequence of SEQ ID NO: 72;
(ii) the VH comprises an amino acid sequence at least 98% identical to the amino acid sequence of SEQ ID NO: 67; and the VL comprises an amino acid sequence at least 98% identical to the amino acid sequence of SEQ ID NO: 72;
(iii) the VH comprises an amino acid sequence at least 98% identical to the amino acid sequence of SEQ ID NO: 71; and the VL comprises an amino acid sequence at least 98% identical to the amino acid sequence of SEQ ID NO: 73; or
(iv) the VH comprises an amino acid sequence at least 98% identical to the amino acid sequence of SEQ ID NO: 71; and the VL comprises an amino acid sequence at least 98% identical to the amino acid sequence of SEQ ID NO: 74.

58. The antibody of claim 55, wherein:
(i) the VH comprises the amino acid sequence of SEQ ID NO: 70; and the VL comprises the amino acid sequence of SEQ ID NO: 72;
(ii) the VH comprises the amino acid sequence of SEQ ID NO: 67; and the VL comprises the amino acid sequence of SEQ ID NO: 72;
(iii) the VH comprises the amino acid sequence of SEQ ID NO: 71; and the VL comprises the amino acid sequence of SEQ ID NO: 73; or
(iv) the VH comprises the amino acid sequence of SEQ ID NO: 71; and the VL comprises the amino acid sequence of SEQ ID NO: 74.

59. The antibody of claim 55, which is a full length antibody, a bispecific antibody, an Fab, an F(ab′)₂, an Fv, or a single chain Fv fragment (scFv).

60. The antibody of claim 55, which comprises a heavy chain constant region selected from IgG1, IgG2, IgG3, or IgG4; and a light chain constant region of kappa or lambda.

61. The antibody of claim 55, which comprises a heavy chain constant region of IgG4 and a light chain constant region of kappa.

62. The antibody of claim 55, which comprises a human IgG4 constant region, comprising a serine to proline substitution at position 228 according to EU numbering.

63. The antibody of claim 55, which comprises a heavy chain constant region comprising the amino acid sequence of SEQ ID NO: 194, or an amino acid sequence at least 95% identical thereto; and a light chain constant region comprising the amino acid sequence of SEQ ID NO: 200, or an amino acid sequence at least 95% identical thereto.

64. The antibody of claim 55, which comprises a heavy chain and a light chain, wherein:
(i) the heavy chain comprises an amino acid sequence at least 95% identical to the amino acid sequence of SEQ ID NO: 173; and the light chain comprises an amino acid sequence at least 95% identical to the amino acid sequence of SEQ ID NO: 175;
(ii) the heavy chain comprises an amino acid sequence at least 95% identical to the amino acid sequence of SEQ ID NO: 170; and the light chain comprises an amino acid sequence at least 95% identical to the amino acid sequence of SEQ ID NO: 175;
(iii) the heavy chain comprises an amino acid sequence at least 95% identical to the amino acid sequence of SEQ ID NO: 174; and the light chain comprises an amino acid sequence at least 95% identical to the amino acid sequence of SEQ ID NO: 176; or
(iv) the heavy chain comprises an amino acid sequence at least 95% identical to the amino acid sequence of SEQ ID NO: 174; and the light chain comprises an amino acid sequence at least 95% identical to the amino acid sequence of SEQ ID NO: 177.

65. The antibody of claim 55, which comprises a heavy chain and a light chain, wherein:
(i) the heavy chain comprises an amino acid sequence at least 98% identical to the amino acid sequence of SEQ ID NO: 173; and the light chain comprises an amino acid sequence at least 98% identical to the amino acid sequence of SEQ ID NO: 175;
(ii) the heavy chain comprises an amino acid sequence at least 98% identical to the amino acid sequence of SEQ ID NO: 170; and the light chain comprises an amino acid sequence at least 98% identical to the amino acid sequence of SEQ ID NO: 175;
(iii) the heavy chain comprises an amino acid sequence at least 98% identical to the amino acid sequence of SEQ ID NO: 174; and the light chain comprises an amino acid sequence at least 98% identical to the amino acid sequence of SEQ ID NO: 176; or
(iv) the heavy chain comprises an amino acid sequence at least 98% identical to the amino acid sequence of SEQ ID NO: 174; and the light chain comprises an amino acid sequence at least 98% identical to the amino acid sequence of SEQ ID NO: 177.

66. The antibody of claim 55, which binds the C-terminus of a tau protein.

67. A nucleic acid encoding the antibody of claim 55.

68. A nucleic acid encoding an antibody that binds to human tau, wherein the encoded antibody comprises a heavy chain variable region (VH) comprising a heavy chain complementarity determining region 1 (HC CDR1) comprising the amino acid sequence of SEQ ID NO: 1144, a HC CDR2 comprising the amino acid sequence of SEQ ID NO: 1145, and a HC CDR3 comprising the amino acid sequence of SEQ ID NO: 410; and a light chain variable region (VL) comprising a light chain complementarity determining region 1 (LC CDR1) comprising the amino acid sequence of SEQ ID NO: 1146, a LC CDR2 comprising the amino acid sequence of SEQ ID NO: 529, and a LC CDR3 comprising the amino acid sequence of SEQ ID NO: 571; and wherein:
(i) the VH comprises the amino acid E at position 1; V at position 5; S at position 7; A at position 9; V at position 11; K at position 12; K at position 19; V at position 20; R at position 67; V at position 68; M at position 70; I at position 76; A at position 79; Y at position 80; M at position 81; E at position 82; R at position 85; D at position 89, L at position 113; and S at position 115, numbered according to SEQ ID NO: 21; and the VL comprises the amino acid S at position 7; T at position 14; Q at position 17; P at position 18; Q at position 42; R at position 44; R at position 50; S at position 68; V at position 88; Y at position 92; and Q at position 105, numbered according to SEQ ID NO: 93;
(ii) the VH comprises the amino acid V at position 5; E at position 6; S at position 7; A at position 9; V at position 11; K at position 12; A at position 16; K at position 19; V at position 20; M at position 48; R at position 67; V at position 68; I at position 70; A at position 76; S at position 77; A at position 79; Y at position 80; M at position 81; E at position 82; R at position 87; T at position 91; and T at position 113, numbered according to SEQ ID NO: 21; and the VL comprises the amino acid S at position 7; T at position 14; Q at position 17; P at position 18; Q at position 42; R at position 44; R at position 50; S at position 68; V at position 88; Y at position 92; and Q at position 105, numbered according to SEQ ID NO: 93;
(iii) the VH comprises the amino acid V at position 5; S at position 7; A at position 9; V at position 11; K at position 12; A at position 16; K at position 19; V at position 20; Q at position 43; M at position 48; R at position 67; V at position 68; M at position 70; T at position 76; S at position 77; R at position 87; and T at position 91, numbered according to SEQ ID NO: 21; and the VL comprises the amino acid I at position 2; S at position 7; S at position 12; T at position 14; P at position 15; Q at position 17; P at position 18; Q at position 50; S at position 68; V at position 88; Y at position 92; Q at position 105; and V at position 109, numbered according to SEQ ID NO: 93; or
(iv) the VH comprises the amino acid V at position 5; S at position 7; A at position 9; V at position 11; K at position 12; A at position 16; K at position 19; V at position 20; Q at position 43; M at position 48; R at position 67; V at position 68; M at position 70; T at position 76; S at position 77; R at position 87; and T at position 91, numbered according to SEQ ID NO: 21; and the VL comprises the amino acid I at position 2; S at position 11; T at position 14; Q at position 17; P at position 18; Q at position 42; R at position 44; P at position 48; R at position 50; S at position 68; A at position 72; N at position 81; V at position 88; and Q at position 105, numbered according to SEQ ID NO: 93.

69. The nucleic acid of claim 68, wherein:
(i) the VH comprises the amino acid sequence of SEQ ID NO: 70, or an amino acid sequence at least 95% identical thereto; and the VL comprises the amino acid sequence of SEQ ID NO: 72, or an amino acid sequence at least 95% identical thereto;
(ii) the VH comprises the amino acid sequence of SEQ ID NO: 67, or an amino acid sequence at least 95% identical thereto; and the VL comprises the amino acid sequence of SEQ ID NO: 72, or an amino acid sequence at least 95% identical thereto;
(iii) the VH comprises the amino acid sequence of SEQ ID NO: 71, or an amino acid sequence at least 95% identical thereto; and the VL comprises the amino acid sequence of SEQ ID NO: 73, or an amino acid sequence at least 95% identical thereto; or
(iv) the VH comprises the amino acid sequence of SEQ ID NO: 71, or an amino acid sequence at least 95% identical thereto; and the VL comprises the amino acid sequence of SEQ ID NO: 74, or an amino acid sequence at least 95% identical thereto.

70. The nucleic acid of claim 68, wherein the encoded antibody comprises a heavy chain and a light chain wherein:
(i) the heavy chain comprises the amino acid sequence of SEQ ID NO: 173, or an amino acid sequence at least 95% identical thereto; and the light chain comprises the amino acid sequence of SEQ ID NO: 175, or an amino acid sequence at least 95% identical thereto;
(ii) the heavy chain comprises the amino acid sequence of SEQ ID NO: 170, or an amino acid sequence at least 95% identical thereto; and the light chain comprises the amino acid sequence of SEQ ID NO: 175, or an amino acid sequence at least 95% identical thereto;
(iii) the heavy chain comprises the amino acid sequence of SEQ ID NO: 174, or an amino acid sequence at least 95% identical thereto; and the light chain comprises the amino acid sequence of SEQ ID NO: 176, or an amino acid sequence at least 95% identical thereto; or
(iv) the heavy chain comprises the amino acid sequence of SEQ ID NO: 174, or an amino acid sequence at least 95% identical thereto; and the light chain comprises the amino acid sequence of SEQ ID NO: 177, or an amino acid sequence at least 95% identical thereto.

71. The nucleic acid of claim 68, wherein:
(i) the nucleotide sequence encoding the VH comprises the nucleotide sequence of SEQ ID NO: 159, or a nucleotide sequence at least 95% identical thereto; and the nucleotide sequence encoding the VL comprises the nucleotide sequence of SEQ ID NO: 161, or a nucleotide sequence at least 95% identical thereto;
(ii) the nucleotide sequence encoding the VH comprises the nucleotide sequence of SEQ ID NO: 156, or a nucleotide sequence at least 95% identical thereto; and the nucleotide sequence encoding the VL comprises the nucleotide sequence of SEQ ID NO: 161, or a nucleotide sequence at least 95% identical thereto;
(iii) the nucleotide sequence encoding the VH comprises the nucleotide sequence of SEQ ID NO: 160, or a nucleotide sequence at least 95% identical thereto; and the nucleotide sequence encoding the VL comprises the nucleotide sequence of SEQ ID NO: 162, or a nucleotide sequence at least 95% identical thereto; or
(iv) the nucleotide sequence encoding the VH comprises the nucleotide sequence of SEQ ID NO: 160, or a nucleotide sequence at least 95% identical thereto; and the nucleotide sequence encoding the VL comprises the nucleotide sequence of SEQ ID NO: 163, or a nucleotide sequence at least 95% identical thereto.

72. The nucleic acid of claim 70, wherein:
(i) the nucleotide sequence encoding the heavy chain comprises the nucleotide sequence of SEQ ID NO: 183, or a nucleotide sequence at least 95% identical thereto; and the nucleotide sequence encoding the light chain comprises the nucleotide sequence of SEQ ID NO: 185, or a nucleotide sequence at least 95% identical thereto;
(ii) the nucleotide sequence encoding the heavy chain comprises the nucleotide sequence of SEQ ID NO: 180, or a nucleotide sequence at least 95% identical thereto; and the nucleotide sequence encoding the light chain comprises the nucleotide sequence of SEQ ID NO: 185, or a nucleotide sequence at least 95% identical thereto;
(iii) the nucleotide sequence encoding the heavy chain comprises the nucleotide sequence of SEQ ID NO: 184, or a nucleotide sequence at least 95% identical thereto; and the nucleotide sequence encoding the light chain comprises the nucleotide sequence of SEQ ID NO: 186, or a nucleotide sequence at least 95% identical thereto; or
(iv) the nucleotide sequence encoding the heavy chain comprises the nucleotide sequence of SEQ ID NO: 184, or a nucleotide sequence at least 95% identical thereto; and the nucleotide sequence encoding the light chain comprises the nucleotide sequence of SEQ ID NO: 187, or a nucleotide sequence at least 95% identical thereto.

73. A pharmaceutical composition comprising the antibody of claim 55, and a pharmaceutically acceptable excipient.

74. A vector comprising the nucleic acid of claim 68.

75. A host cell comprising the nucleic acid of claim 68.

76. A method of producing an antibody, the method comprising culturing the host cell of claim 74 under conditions suitable for gene expression, thereby producing the antibody.

77. A method of delivering an antibody that binds to tau to a subject, comprising administering an effective amount of the antibody of claim 55 to the subject, thereby delivering the antibody that binds to tau to the subject.


SEQUENCE LISTING (5348 sequences)

SEQ ID NO: 1 - protein, 19 aa, synthetic construct
MNFGLSLIFLVLVLKGVQC

SEQ ID NO: 2 - protein, 19 aa, synthetic construct
MKLPVRLLVLMFWIPASSS

SEQ ID NO: 3 - protein, 6 aa, synthetic construct
SGGGGS

SEQ ID NO: 4 - protein, 113 aa, synthetic construct
QVQLQQPGAELVKPGASVKLSCKASDYTFTNYWMHWVKQRPGRGLEWIGRIDPNSGGTRYNEKFKNKATLTVDKPSSTAYMHLSSLTSEDSAVYYCAGDFDVWGTGTTVTVSS

SEQ ID NO: 5-6 - skipped (2 entries)

SEQ ID NO: 7 - DNA, 354 bp, synthetic construct
  source 1..354: mol_type = other DNA
caagtgcagctgcagcagcctggcaccgagctggtgaaacctggatctagcgtgaatctgagctgcaaggccagcggctttaccttcaccagatactggatgcactgggtcaaggaacggccaggccacggcctggaatggatcggcaatatcaaccccaacaacggcggaacagatttcaacgagaagttcaagaacaaggctacactgaccgtgcacaaaagctccaccaccgtgttcatccagctgagctctctgacaagcgaggacagcgccgtgtactattgtgccagaggcaccggcaccggcgccatggactactggggccagggaacatctgtgacagtgtccagc

SEQ ID NO: 8 - protein, 441 aa, synthetic construct
QVQLQQPGTELVKPGSSVNLSCKASGFTFTRYWMHWVKERPGHGLEWIGNINPNNGGTDFNEKFKNKATLTVHKSSTTVFIQLSSLTSEDSAVYYCARGTGTGAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPG

SEQ ID NO: 9 - protein, 115 aa, synthetic construct
EVQLVESGGALVQPGGSLSLSCAASGFTFTDYYMSWVRQPPGKALEWLALIRNKAKGFTTEYSASVKGRFTISRDNSQSILLFQMNDLRADDSATYYCVRDINYWGQGTTLTVSS

SEQ ID NO: 10 - DNA, 1323 bp, synthetic construct
  source 1..1323: mol_type = other DNA
caagtgcagctgcagcagcctggcaccgagctggtgaaacctggatctagcgtgaatctgagctgcaaggccagcggctttaccttcaccagatactggatgcactgggtcaaggaacggccaggccacggcctggaatggatcggcaatatcaaccccaacaacggcggaacagatttcaacgagaagttcaagaacaaggctacactgaccgtgcacaaaagctccaccaccgtgttcatccagctgagctctctgacaagcgaggacagcgccgtgtactattgtgccagaggcaccggcaccggcgccatggactactggggccagggaacatctgtgacagtgtccagcgccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggt

SEQ ID NO: 11 - DNA, 336 bp, synthetic construct
  source 1..336: mol_type = other DNA
gatgtggtgatgacacagacccctctgagcctgcctgtgtccctcggcgaccaggccagcatcagctgtagaagcagccaatctctggtgcacaacaatggcatcacctacctgtactggtatctgcagaaacctggccagagccccaagctgctgatctaccgggtgtccaatcggttcagcggagtgccagatagatttggcggatctggcagcggcaccgacttcaccctgaagatctctagagtcgaggccgaggacctgggcgtgtacttctgcttccagggcacacacgtgcccagaaccttcggcggcggaacaaagctggaaatcaag

SEQ ID NO: 12 - protein, 219 aa, synthetic construct
DVVMTQTPLSLPVSLGDQASISCRSSQSLVHNNGITYLYWYLQKPGQSPKLLIYRVSNRFSGVPDRFGGSGSGTDFTLKISRVEAEDLGVYFCFQGTHVPRTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 13 - DNA, 657 bp, synthetic construct
  source 1..657: mol_type = other DNA
gatgtggtgatgacacagacccctctgagcctgcctgtgtccctcggcgaccaggccagcatcagctgtagaagcagccaatctctggtgcacaacaatggcatcacctacctgtactggtatctgcagaaacctggccagagccccaagctgctgatctaccgggtgtccaatcggttcagcggagtgccagatagatttggcggatctggcagcggcaccgacttcaccctgaagatctctagagtcgaggccgaggacctgggcgtgtacttctgcttccagggcacacacgtgcccagaaccttcggcggcggaacaaagctggaaatcaagcgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 14 - skipped

SEQ ID NO: 15 - DNA, 3962 bp, synthetic construct
  source 1..3962: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagcttcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaaccggtgccgccaccatgaacttcgggctcagcttgattttccttgtccttgttttaaaaggtgtccagtgtcaagtgcagctgcagcagcctggcaccgagctggtgaaacctggatctagcgtgaatctgagctgcaaggccagcggctttaccttcaccagatactggatgcactgggtcaaggaacggccaggccacggcctggaatggatcggcaatatcaaccccaacaacggcggaacagatttcaacgagaagttcaagaacaaggctacactgaccgtgcacaaaagctccaccaccgtgttcatccagctgagctctctgacaagcgaggacagcgccgtgtactattgtgccagaggcaccggcaccggcgccatggactactggggccagggaacatctgtgacagtgtccagcgccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtagaaagaggcgagagggcagaggaagtcttctaacatgcggtgacgtggaggagaatcccggccctatgaagttgcctgttaggctgttggtgctgatgttctggattcctgcttccagcagtgatgtggtgatgacacagacccctctgagcctgcctgtgtccctcggcgaccaggccagcatcagctgtagaagcagccaatctctggtgcacaacaatggcatcacctacctgtactggtatctgcagaaacctggccagagccccaagctgctgatctaccgggtgtccaatcggttcagcggagtgccagatagatttggcggatctggcagcggcaccgacttcaccctgaagatctctagagtcgaggccgaggacctgggcgtgtacttctgcttccagggcacacacgtgcccagaaccttcggcggcggaacaaagctggaaatcaagcgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttaactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggcctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 16 - protein, 323 aa, Mus sp.
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPG

SEQ ID NO: 17 - DNA, 321 bp, Mus sp.
  source 1..321: mol_type = genomic DNA
cgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 18 - protein, 107 aa, Mus sp.
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 19-20 - skipped (2 entries)

SEQ ID NO: 21 - protein, 118 aa, synthetic construct
QVQLQQPGTELVKPGSSVNLSCKASGFTFTRYWMHWVKERPGHGLEWIGNINPNNGGTDFNEKFKNKATLTVHKSSTTVFIQLSSLTSEDSAVYYCARGTGTGAMDYWGQGTSVTVSS

SEQ ID NO: 22 - protein, 118 aa, synthetic construct
QVQLQQPGTELVKPGASVKLSCKASGYTFTIFWMHWVKQRPGHGLEWIGKINPNNGGGDYNEKFKSKATLTVDKSSTTAYLQLSSLTSEDSAVYYCARGTGTGAMDYWGQGTSVTVSS

SEQ ID NO: 23 - protein, 118 aa, synthetic construct
QVQLQQPGTELVKPGASVKLSCKASGYTFTRFWMHWVKQRPGQGLEWIGNINPNNGGTDNNERFKSKATLTVDRSSSTAYMQLSSLTSEDSAVYYCARGTGTGAMDYWGQGTSVTVSS

SEQ ID NO: 24-31 - skipped (8 entries)

SEQ ID NO: 32 - protein, 22 aa, synthetic construct
  SITE 5: note = Phosphorylated residue
  SITE 8: note = Phosphorylated residue
  SITE 9: note = Phosphorylated residue
  SITE 10: note = Phosphorylated residue
  SITE 12: note = Phosphorylated residue
  SITE 18: note = Phosphorylated residue
GSGSSNVSSTGSIDLVDSPQLA

SEQ ID NO: 33 - protein, 22 aa, synthetic construct
  SITE 18: note = Phosphorylated residue
GSGSSNVSSTGSIDLVDSPQLA

SEQ ID NO: 34 - protein, 22 aa, synthetic construct
  SITE 12: note = Phosphorylated residue
  SITE 18: note = Phosphorylated residue
GSGSSNVSSTGSIDLVDSPQLA

SEQ ID NO: 35-50 - skipped (16 entries)

SEQ ID NO: 51 - protein, 119 aa, synthetic construct
QITLKESGPGILQSSQTLSLTCSFSGFSLSTSAMGVSWIRQPSGEGLEWLAHIYWDDDKRYNPSLKSRLTISKDTSRNQVFLKITSVDTADTATYYCARRRRGYGMDYWGQGTSVTVSS

SEQ ID NO: 52-63 - skipped (12 entries)

SEQ ID NO: 64 - protein, 10 aa, synthetic construct
GFTFTRYWMH

SEQ ID NO: 65 - protein, 445 aa, synthetic construct
QVQLQQPGTELVKPGSSVNLSCKASGFTFTRYWMHWVKERPGHGLEWIGNINPNNGGTDFNEKFKNKATLTVHKSSTTVFIQLSSLTSEDSAVYYCARGTGTGAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 66 - protein, 219 aa, synthetic construct
DVVMTQTPLSLPVSLGDQASISCRSSQSLVHNNGITYLYWYLQKPGQSPKLLIYRVSNRFSGVPDRFGGSGSGTDFTLKISRVEAEDLGVYFCFQGTHVPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 118 aa, synthetic construct
QVQLVESGAEVKKPGASVKVSCKASGFTFTRYWMHWVKERPGHGLEWMGNINPNNGGTDFNEKFKNRVTITVHKSASTAYMELSSLRSEDTAVYYCARGTGTGAMDYWGQGTTVTVSS

SEQ ID NO: 68 - protein, 118 aa, synthetic construct
QVQLVQSGAEVKKPGASVKVSCKASGFTFTRYWMHWVRERPGHGLEWIGNINPNNGGTDFNEKFKNRVTMTVHKSISTAYMELSSLRSDDSAVYYCARGTGTGAMDYWGQGTLVTVSS

SEQ ID NO: 69 - protein, 118 aa, synthetic construct
QVQLVQSGAEVKKSGASVKVSCKASGFTFTRYWMHWVRQAPGQGLEWIGNINPNNGGTDFNEKFKNRVTLIRDTSTTTVFIQLTSLTSEDSAVYYCARGTGTGAMDYWGQGTLVTVSS

SEQ ID NO: 70 - protein, 118 aa, synthetic construct
EVQLVQSGAEVKKPGSSVKVSCKASGFTFTRYWMHWVKERPGHGLEWIGNINPNNGGTDFNEKFKNRVTMTVHKSITTAYMELSRLTSDDSAVYYCARGTGTGAMDYWGQGTLVSVSS

SEQ ID NO: 71 - protein, 118 aa, synthetic construct
QVQLVQSGAEVKKPGASVKVSCKASGFTFTRYWMHWVKERPGQGLEWMGNINPNNGGTDFNEKFKNRVTMTVHKSTSTVFIQLSSLRSEDTAVYYCARGTGTGAMDYWGQGTSVTVSS

SEQ ID NO: 72 - protein, 112 aa, synthetic construct
DVVMTQSPLSLPVTLGQPASISCRSSQSLVHNNGITYLYWYQQRPGQSPRLLIYRVSNRFSGVPDRFSGSGSGTDFTLKISRVEAEDVGVYYCFQGTHVPRTFGQGTKLEIK

SEQ ID NO: 73 - protein, 112 aa, synthetic construct
DIVMTQSPLSLSVTPGQPASISCRSSQSLVHNNGITYLYWYLQKPGQSPQLLIYRVSNRFSGVPDRFSGSGSGTDFTLKISRVEAEDVGVYYCFQGTHVPRTFGQGTKVEIK

SEQ ID NO: 74 - protein, 112 aa, synthetic construct
DIVMTQTPLSSPVTLGQPASISCRSSQSLVHNNGITYLYWYQQRPGQPPRLLIYRVSNRFSGVPDRFSGSGAGTDFTLKINRVEAEDVGVYFCFQGTHVPRTFGQGTKLEIK

SEQ ID NO: 75 - protein, 112 aa, synthetic construct
DIEMTQSPLSLPVTLGQPASISCRSSQSLVHNNGITYLYWYQQRPGQSPRRLIYRVSNRFSGVPDRFSGSGSGTDFTLKISRVEAEDVGVYFCFQGTHVPRTFGGGTKLEIK

SEQ ID NO: 76 - protein, 112 aa, synthetic construct
EIVLTQSPATLSLSPGERATLSCRSSQSLVHNNGITYLYWYLQKPGQAPKLLIYRVSNRFSGIPDRFSGSGPGTDFTLTISSLEPEDLAVYFCFQGTHVPRTFGGGTKLEIK

SEQ ID NO: 77 - skipped

SEQ ID NO: 78 - protein, 107 aa, synthetic construct
DIQMTQSPASLSASVGETVTITCRASGNIHNYLAWYQQRQGKSPQLLVYNAKTLPDGVPSRFSGSGSGTQYSLKINSLQPEDFGSYCCQHFWSTPLTFGAGTKLELK

SEQ ID NO: 79-82 - skipped (4 entries)

SEQ ID NO: 83 - protein, 113 aa, synthetic construct
DIVMSQSPSSLAVSVGEKVTMSCKSSQSLLYSTNQENYLAWYQQKPGQSPKLLIYWASSRESGVPDRFTGSGSGTDFTLTISSVKAEDLAVYYCQQYYSYPRTFGGGTKLEIK

SEQ ID NO: 84-92 - skipped (9 entries)

SEQ ID NO: 93 - protein, 112 aa, synthetic construct
DVVMTQTPLSLPVSLGDQASISCRSSQSLVHNNGITYLYWYLQKPGQSPKLLIYRVSNRFSGVPDRFGGSGSGTDFTLKISRVEAEDLGVYFCFQGTHVPRTFGGGTKLEIK

SEQ ID NO: 94 - protein, 112 aa, synthetic construct
DVVMTQTPLSLPVSLGDHASISCRSSQSLVHSNGITHLYWYLQRPGQTPKLLIYRVSNRFSGVPDRFSGSGSGTDFTLKISSVEAEDLGVYFCFQGTHVPRTFGGGTKLEIE

SEQ ID NO: 95 - protein, 112 aa, synthetic construct
DVVMTQTPLSLPVSLGDQASISCRSSQSLVHSNGNTHLYWYLQKPGQSPKLLIYRVSSRFSGVPDRFSGSGSGTDFTLKISRVEAEDLGVYFCFQGTHVPRTFGGGTKLEIK

SEQ ID NO: 96-121 - skipped (26 entries)

SEQ ID NO: 122 - protein, 107 aa, synthetic construct
SIVMTQTPKFLLVSAGDRVTITCKASQSVSNDVAWYQQKPGQSPKLLIYYASNRCTGVPDRFTGSGYGTDFTFTISTVQAEDLAVYFCQQDYRSPLTFGAGTKLELK

SEQ ID NO: 123-136 - skipped (14 entries)

SEQ ID NO: 137 - DNA, 2211 bp, Adeno-associated virus 9
  source 1..2211: mol_type = genomic DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 138 - protein, 736 aa, Adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 139-149 - skipped (11 entries)

SEQ ID NO: 150 - DNA, 339 bp, synthetic construct
  source 1..339: mol_type = other DNA
caggtccaactgcagcagcctggggctgagcttgtgaagcctggggcttcagtgaagctgtcctgcaaggcttctgactacaccttcaccaactactggatgcactgggtgaagcagaggcctggacgaggccttgagtggataggaaggattgatcctaatagtggtggtactaggtacaatgagaagttcaagaacaaggccacactgactgttgacaaaccctccagcacagcctacatgcatctcagcagcctgacatctgaggactctgcggtctattattgtgcaggggacttcgatgtctggggcacagggaccacggtcaccgtctcctca

SEQ ID NO: 151-154 - skipped (4 entries)

SEQ ID NO: 155 - DNA, 345 bp, synthetic construct
  source 1..345: mol_type = other DNA
gaggtgcagctggtggagtctggaggagccttggtacagcctgggggttctctgagtctctcctgtgcagcttctggattcaccttcactgattactacatgagctgggtccgccagcctccagggaaggcacttgagtggctggctttgattagaaacaaagctaaaggtttcacaacagaatacagtgcatctgtgaagggtcggttcaccatctccagagataattcccaaagcatcctcctttttcaaatgaatgacctgagagctgacgacagtgccacttattactgtgtaagagatataaactactggggccaaggcaccactctcacagtctcctca

SEQ ID NO: 156 - DNA, 354 bp, synthetic construct
  source 1..354: mol_type = other DNA
caagtgcagctggtggagagcggcgccgaggtgaagaagcctggcgctagcgtgaaggtgagctgcaaggctagcggcttcaccttcacaagatactggatgcactgggtgaaggagagacctggccacggcctggagtggatgggcaacatcaaccctaacaacggcggcaccgacttcaacgagaagttcaagaacagagtgaccatcaccgtgcacaagagcgcgtcgaccgcttacatggagctgagcagcctgagaagcgaggacaccgccgtgtactactgcgctagaggcaccggcaccggcgccatggactactggggccaaggcaccaccgtgaccgtctcgtcc

SEQ ID NO: 157 - DNA, 354 bp, synthetic construct
  source 1..354: mol_type = other DNA
caagtgcagctggtgcagagcggcgccgaggtgaagaagcctggcgctagcgtgaaggtgagctgcaaggctagcggcttcaccttcacaagatactggatgcactgggtgagagagagacctggccacggcctggagtggatcggcaacatcaaccctaacaacggcggcaccgacttcaacgagaagttcaagaacagagtgaccatgaccgtgcacaagagcatcagcaccgcctacatggagctgagcagcctgagaagcgacgacagcgccgtgtactactgcgctagaggcaccggcaccggcgccatggactactggggccaaggcaccctggtgacggtaagctcc

SEQ ID NO: 158 - DNA, 354 bp, synthetic construct
  source 1..354: mol_type = other DNA
caagtgcagctggtgcagagcggcgccgaggtgaagaagagcggcgctagcgtgaaggtgagctgcaaggctagcggcttcaccttcacaagatactggatgcactgggtgagacaagcccctggccaaggcctggagtggatcggcaacatcaaccctaacaacggcggcaccgacttcaacgagaagttcaagaacagagtgaccctgatcagagacacaagcaccaccaccgtgttcattcagctgacaagcctgacaagcgaggacagcgccgtgtactactgcgctagaggcaccggcaccggcgccatggactactggggccaaggcaccctggtgacggtaagctcc

SEQ ID NO: 159 - DNA, 354 bp, synthetic construct
  source 1..354: mol_type = other DNA
gaggtgcagctggtgcagagcggcgccgaggtgaagaagcctggcagcagcgtgaaggtgagctgcaaggctagcggcttcaccttcacaagatactggatgcactgggtgaaggagagacctggccacggcctggagtggatcggcaacatcaaccctaacaacggcggcaccgacttcaacgagaagttcaagaacagagtgaccatgaccgtgcacaagagcatcaccaccgcctacatggagctgagccggttaacctccgacgacagcgccgtgtactactgcgctagaggcaccggcaccggcgccatggactactggggccaaggcaccctggtgagcgtgagcagc

SEQ ID NO: 160 - DNA, 354 bp, synthetic construct
  source 1..354: mol_type = other DNA
caagtgcagctggtgcagagcggcgccgaggtgaagaagcctggcgctagcgtgaaggtgagctgcaaggctagcggcttcaccttcacaagatactggatgcactgggtgaaggagagacctggccaaggcctggagtggatgggcaacatcaaccctaacaacggcggcaccgacttcaacgagaagttcaagaacagagtgaccatgaccgtgcacaagagcacaagcaccgtgttcattcagctgagcagcctgagaagcgaggacaccgccgtgtactactgcgctagaggcaccggcaccggcgccatggactactggggccaaggcacaagcgtgacggtaagctcc

SEQ ID NO: 161 - DNA, 336 bp, synthetic construct
  source 1..336: mol_type = other DNA
gacgtggtgatgacacagagccctctgagcctgcctgtgaccctgggacagcctgctagcatcagctgcagaagctctcagagcctggtgcacaacaacggcatcacctacctgtactggtatcagcagagacctggacagagccctagactgctgatctacagagtgagcaacagattcagcggcgtgcctgacagattctccggctccggcagcggcaccgacttcaccctgaagatcagcagagtggaggccgaggacgtgggcgtgtactactgctttcaaggcacccatgtccctagaaccttcggccaaggcaccaagctggagatcaag

SEQ ID NO: 162 - DNA, 336 bp, synthetic construct
  source 1..336: mol_type = other DNA
gacatcgtgatgacacagagccctctgagcctgagcgtgacccctggacagcctgctagcatcagctgcagaagctctcagagcctggtgcacaacaacggcatcacctacctgtactggtacctgcagaagcctggacagagccctcagctgctgatctacagagtgagcaacagattcagcggcgtgcctgacagattctccggctccggcagcggcaccgacttcaccctgaagatcagcagagtggaggccgaggacgtgggcgtgtactactgctttcaaggcacccatgtccctagaaccttcggccaaggcaccaaggtggagatcaag

SEQ ID NO: 163 - DNA, 336 bp, synthetic construct
  source 1..336: mol_type = other DNA
gacatcgtgatgacacagacccctctcagcagccctgtgaccctaggacagcctgctagcatcagctgcagaagctctcagagcctggtgcacaacaacggcatcacctacctgtactggtatcagcagagacctggacagcctcctagactgctgatctacagagtgagcaacagattcagcggagtacctgacagatttagcggttccggcgccggcaccgacttcaccctgaagatcaacagagtggaggccgaggacgtgggcgtgtacttctgctttcaaggcacccatgtccctagaaccttcggccaaggcaccaagctggagatcaag

SEQ ID NO: 164 - DNA, 336 bp, synthetic construct
  source 1..336: mol_type = other DNA
gacatcgagatgacacagagccctctgagcctgcctgtgaccctgggacagcctgctagcatcagctgcagaagctctcagagcctggtgcacaacaacggcatcacctacctgtactggtatcagcagagacctggacagagccctagaagactgatctacagagtgagcaacagattcagcggcgtgcctgacagattctccggctccggcagcggcaccgacttcaccctgaagatcagcagagtggaggccgaggacgtgggcgtgtacttctgctttcaaggcacccatgtccctagaaccttcggcggcggcaccaagctggagatcaag

SEQ ID NO: 165 - DNA, 336 bp, synthetic construct
  source 1..336: mol_type = other DNA
gagatcgtgctgacacagagccctgcgacactgagcctgagccctggcgagagagctacgctgagctgccgcagctctcagagcctggtgcacaacaacggcatcacctacctgtactggtacctgcagaagcctggccaagcccctaagctgctgatctacagagtgagcaacagattcagcggcatccctgacagattctccggctccggccctggcaccgacttcaccctgaccatcagcagcctggagcctgaggacctggccgtgtacttctgcttccaaggcacccatgtacctagaaccttcggcggcggcaccaagctggagatcaag

SEQ ID NO: 166 - skipped

SEQ ID NO: 167 - DNA, 354 bp, synthetic construct
  source 1..354: mol_type = other DNA
caggtccaactgcagcagcctgggactgaactggtgaagcctgggtcttcagtgaacctgtcctgcaaggcttctggcttcaccttcaccaggtactggatgcactgggtgaaggagaggcctggacatggccttgagtggattggaaatattaatcctaacaatggtggtactgacttcaatgagaagttcaagaacaaggccacactgactgtacacaagtcctccaccacagtcttcatccaactcagcagcctgacatctgaggactctgcggtctattattgtgcaagaggaactgggacgggagctatggactactggggtcaaggaacctcagtcaccgtctcctca

SEQ ID NO: 168 - DNA, 354 bp, synthetic construct
  source 1..354: mol_type = other DNA
caggtccaactgcagcagcctgggactgaactggtgaagcctggggcttcagtgaagctgtcctgcaaggcttctggctacaccttcaccatcttctggatgcactgggtgaagcagaggcctggacatggccttgagtggattggaaagattaatcctaacaatggaggtggtgactacaatgagaaattcaagagtaaggccacattgactgtagacaaatcctccaccacagcctacttgcagctcagcagcctgacatctgaggactctgcggtctattattgtgcaagaggaactgggacgggagctatggactactggggtcaaggaacctcagtcaccgtctcctca

SEQ ID NO: 169 - DNA, 354 bp, synthetic construct
  source 1..354: mol_type = other DNA
caggtccaactgcagcagcctgggactgaactggtgaagcctggggcttcagtgaagctgtcctgcaaggcttctggctacaccttcaccaggttctggatgcactgggtgaagcagaggcctggacaaggccttgagtggattggaaatattaatcctaacaatggtggtactgacaataatgagaggttcaagagcaaggccacactgactgtagacagatcctccagcacagcctacatgcagctcagcagcctgacatctgaggactctgcggtctattattgtgcaagaggaactgggacgggagctatggactactggggtcaaggaacctcagtcaccgtctcctca

SEQ ID NO: 170 - protein, 445 aa, synthetic construct
QVQLVESGAEVKKPGASVKVSCKASGFTFTRYWMHWVKERPGHGLEWMGNINPNNGGTDFNEKFKNRVTITVHKSASTAYMELSSLRSEDTAVYYCARGTGTGAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 171 - protein, 445 aa, synthetic construct
QVQLVQSGAEVKKPGASVKVSCKASGFTFTRYWMHWVRERPGHGLEWIGNINPNNGGTDFNEKFKNRVTMTVHKSISTAYMELSSLRSDDSAVYYCARGTGTGAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 172 - protein, 445 aa, synthetic construct
QVQLVQSGAEVKKSGASVKVSCKASGFTFTRYWMHWVRQAPGQGLEWIGNINPNNGGTDFNEKFKNRVTLIRDTSTTTVFIQLTSLTSEDSAVYYCARGTGTGAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 173 - protein, 445 aa, synthetic construct
EVQLVQSGAEVKKPGSSVKVSCKASGFTFTRYWMHWVKERPGHGLEWIGNINPNNGGTDFNEKFKNRVTMTVHKSITTAYMELSRLTSDDSAVYYCARGTGTGAMDYWGQGTLVSVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 174 - protein, 445 aa, synthetic construct
QVQLVQSGAEVKKPGASVKVSCKASGFTFTRYWMHWVKERPGQGLEWMGNINPNNGGTDFNEKFKNRVTMTVHKSTSTVFIQLSSLRSEDTAVYYCARGTGTGAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 175 - protein, 219 aa, synthetic construct
DVVMTQSPLSLPVTLGQPASISCRSSQSLVHNNGITYLYWYQQRPGQSPRLLIYRVSNRFSGVPDRFSGSGSGTDFTLKISRVEAEDVGVYYCFQGTHVPRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 176 - protein, 219 aa, synthetic construct
DIVMTQSPLSLSVTPGQPASISCRSSQSLVHNNGITYLYWYLQKPGQSPQLLIYRVSNRFSGVPDRFSGSGSGTDFTLKISRVEAEDVGVYYCFQGTHVPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 177 - protein, 219 aa, synthetic construct
DIVMTQTPLSSPVTLGQPASISCRSSQSLVHNNGITYLYWYQQRPGQPPRLLIYRVSNRFSGVPDRFSGSGAGTDFTLKINRVEAEDVGVYFCFQGTHVPRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 178 - protein, 219 aa, synthetic construct
DIEMTQSPLSLPVTLGQPASISCRSSQSLVHNNGITYLYWYQQRPGQSPRRLIYRVSNRFSGVPDRFSGSGSGTDFTLKISRVEAEDVGVYFCFQGTHVPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 179 - protein, 219 aa, synthetic construct
EIVLTQSPATLSLSPGERATLSCRSSQSLVHNNGITYLYWYLQKPGQAPKLLIYRVSNRFSGIPDRFSGSGPGTDFTLTISSLEPEDLAVYFCFQGTHVPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 180 - DNA, 1335 bp, synthetic construct
  source 1..1335: mol_type = other DNA
caagtgcagctggtggagagcggcgccgaggtgaagaagcctggcgctagcgtgaaggtgagctgcaaggctagcggcttcaccttcacaagatactggatgcactgggtgaaggagagacctggccacggcctggagtggatgggcaacatcaaccctaacaacggcggcaccgacttcaacgagaagttcaagaacagagtgaccatcaccgtgcacaagagcgcgtcgaccgcttacatggagctgagcagcctgagaagcgaggacaccgccgtgtactactgcgctagaggcaccggcaccggcgccatggactactggggccaaggcaccaccgtgaccgtctcgtccgctagcacgaaaggtccaagcgttttccctctagccccttgcagcagaagcacaagcgagagcaccgccgccctgggctgccttgtaaaagattacttccctgagcctgtgaccgtgagttggaacagcggcgccctgacaagcggcgtgcacaccttccctgccgtgctgcagagcagcggcctgtacagcctgagcagcgtggtgaccgtgccaagcagcagcctgggcaccaagacctacacctgcaacgtggaccacaagcctagcaacaccaaggtggacaagagagtggagagcaagtacggtcctccgtgtcccccgtgccctgcccctgagttcctgggcggcccttcggtgtttctgtttccacctaagcctaaggacaccctgatgatcagcagaacccctgaggtgacctgcgtggtggtggacgtgagccaagaggaccctgaggtgcagttcaactggtacgtggacggcgtggaggtgcacaacgccaagaccaagcctagagaggagcagttcaacagcacctacagagtggtgagcgtgctgaccgtgctgcaccaagactggctgaacggcaaggagtacaagtgcaaggtgagcaacaagggcctgcctagttccattgagaagaccatcagcaaggccaagggacagcctagagagcctcaagtgtacaccctgcctcctagccaagaggagatgaccaagaaccaagtgagcctgacctgcctggtgaagggattctaccctagcgacatcgccgtggagtgggagagcaacggacagcctgagaacaactacaagaccacccctcctgtgctggacagcgacggcagcttcttcctgtacagcagactgaccgtggacaagagcagatggcaagagggcaacgtgttcagctgcagcgtgatgcacgaggccctgcacaaccactacacacagaagagcctgagcctgagcctgggcaag

SEQ ID NO: 181 - DNA, 1335 bp, synthetic construct
  source 1..1335: mol_type = other DNA
caagtgcagctggtgcagagcggcgccgaggtgaagaagcctggcgctagcgtgaaggtgagctgcaaggctagcggcttcaccttcacaagatactggatgcactgggtgagagagagacctggccacggcctggagtggatcggcaacatcaaccctaacaacggcggcaccgacttcaacgagaagttcaagaacagagtgaccatgaccgtgcacaagagcatcagcaccgcctacatggagctgagcagcctgagaagcgacgacagcgccgtgtactactgcgctagaggcaccggcaccggcgccatggactactggggccaaggcaccctggtgacggtaagctccgctagcaccaagggtcctagtgtatttcccctagccccttgcagcagaagcacaagcgagagcaccgccgccctgggctgcttggtgaaggactacttccctgagcctgtcacagtgtcctggaatagcggcgccctgacaagcggcgtgcacaccttccctgccgtgctgcagagcagcggcctgtacagcctgagcagcgtggtgaccgtgccttcgtcgagcctgggcaccaagacctacacctgcaacgtggaccacaagcctagcaacaccaaggtggacaagagagtggagagcaagtacggtcctccgtgtcccccgtgccctgcccctgagttcctgggcggccctagcgtgttcctatttccacctaagcctaaggacaccctgatgatcagcagaacccctgaggtgacctgcgtggtggtggacgtgagccaagaggaccctgaggtgcagttcaactggtacgtggacggcgtggaggtgcacaacgccaagaccaagcctagagaggagcagttcaacagcacctacagagtggtgagcgtgctgaccgtgctgcaccaagactggctgaacggcaaggagtacaagtgcaaggtgagcaacaagggcctgcctagcagtatcgagaagaccatcagcaaggccaagggacagcctagagagcctcaagtgtacaccctgcctcctagccaagaggagatgaccaagaaccaagtgagcctgacctgcctggtaaaaggtttctaccctagcgacatcgccgtggagtgggagagcaacggacagcctgagaacaactacaagaccacccctcctgtgctggacagcgacggcagcttcttcctgtacagcagactgaccgtggacaagagcagatggcaagagggcaacgtgttcagctgcagcgtgatgcacgaggccctgcacaaccactacacacagaagagcctgagcctgagcctgggcaag

SEQ ID NO: 182 - DNA, 1335 bp, synthetic construct
  source 1..1335: mol_type = other DNA
caagtgcagctggtgcagagcggcgccgaggtgaagaagagcggcgctagcgtgaaggtgagctgcaaggctagcggcttcaccttcacaagatactggatgcactgggtgagacaagcccctggccaaggcctggagtggatcggcaacatcaaccctaacaacggcggcaccgacttcaacgagaagttcaagaacagagtgaccctgatcagagacacaagcaccaccaccgtgttcattcagctgacaagcctgacaagcgaggacagcgccgtgtactactgcgctagaggcaccggcaccggcgccatggactactggggccaaggcaccctggtgacggtaagctccgctagcaccaagggtcctagtgtatttcccctagccccttgcagcagaagcacaagcgagagcaccgccgccctgggctgcttggtgaaggactacttccctgagcctgtcacagtgtcctggaatagcggcgccctgacaagcggcgtgcacaccttccctgccgtgctgcagagcagcggcctgtacagcctgagcagcgtggtgaccgtgccttcgtcgagcctgggcaccaagacctacacctgcaacgtggaccacaagcctagcaacaccaaggtggacaagagagtggagagcaagtacggtcctccgtgtcccccgtgccctgcccctgagttcctgggcggccctagcgtgttcctatttccacctaagcctaaggacaccctgatgatcagcagaacccctgaggtgacctgcgtggtggtggacgtgagccaagaggaccctgaggtgcagttcaactggtacgtggacggcgtggaggtgcacaacgccaagaccaagcctagagaggagcagttcaacagcacctacagagtggtgagcgtgctgaccgtgctgcaccaagactggctgaacggcaaggagtacaagtgcaaggtgagcaacaagggcctgcctagcagtatcgagaagaccatcagcaaggccaagggacagcctagagagcctcaagtgtacaccctgcctcctagccaagaggagatgaccaagaaccaagtgagcctgacctgcctggtaaaaggtttctaccctagcgacatcgccgtggagtgggagagcaacggacagcctgagaacaactacaagaccacccctcctgtgctggacagcgacggcagcttcttcctgtacagcagactgaccgtggacaagagcagatggcaagagggcaacgtgttcagctgcagcgtgatgcacgaggccctgcacaaccactacacacagaagagcctgagcctgagcctgggcaag

SEQ ID NO: 183 - DNA, 1335 bp, synthetic construct
  source 1..1335: mol_type = other DNA
gaggtgcagctggtgcagagcggcgccgaggtgaagaagcctggcagcagcgtgaaggtgagctgcaaggctagcggcttcaccttcacaagatactggatgcactgggtgaaggagagacctggccacggcctggagtggatcggcaacatcaaccctaacaacggcggcaccgacttcaacgagaagttcaagaacagagtgaccatgaccgtgcacaagagcatcaccaccgcctacatggagctgagccggttaacctccgacgacagcgccgtgtactactgcgctagaggcaccggcaccggcgccatggactactggggccaaggcaccctggtgagcgtgagcagcgctagcaccaagggtcctagtgtatttcccctagccccttgcagcagaagcacaagcgagagcaccgccgccctgggctgcttggtgaaggactacttccctgagcctgtgaccgtgagctggaacagcggcgccctgacaagcggcgtgcacaccttccctgccgtgctgcagagcagcggcctgtacagcctgagcagcgtggtgaccgtgccttcgtcgagcctgggcaccaagacctacacctgcaacgtggaccacaagcctagcaacaccaaggtggacaagagagtggagagcaagtacggtcctccgtgtcccccgtgccctgcccctgagttcctgggcggccctagcgtgttcctatttccacctaagcctaaggacaccctgatgatcagcagaacccctgaggtgacctgcgtggtggtggacgtgagccaagaggaccctgaggtgcagttcaactggtacgtggacggcgtggaggtgcacaacgccaagaccaagcctagagaggagcagttcaacagcacctacagagtggtgagcgtgctgaccgtgctgcaccaagactggctgaacggcaaggagtacaagtgcaaggtgagcaacaagggcctgcctagcagtatcgagaagaccatcagcaaggccaagggacagcctagagagcctcaagtgtacaccctgcctcctagccaagaggagatgaccaagaaccaagtgagcctgacctgcctggtaaaaggtttctaccctagcgacatcgccgtggagtgggagagcaacggacagcctgagaacaactacaagaccacccctcctgtgctggacagcgacggcagcttcttcctgtactcccgcctgacggttgacaagagcagatggcaagagggcaacgtgttcagctgcagcgtgatgcacgaggccctgcacaaccactacacacagaagagcctgagcctgagcctgggcaag

SEQ ID NO: 184 - DNA, 1335 bp, synthetic construct
  source 1..1335: mol_type = other DNA
caagtgcagctggtgcagagcggcgccgaggtgaagaagcctggcgctagcgtgaaggtgagctgcaaggctagcggcttcaccttcacaagatactggatgcactgggtgaaggagagacctggccaaggcctggagtggatgggcaacatcaaccctaacaacggcggcaccgacttcaacgagaagttcaagaacagagtgaccatgaccgtgcacaagagcacaagcaccgtgttcattcagctgagcagcctgagaagcgaggacaccgccgtgtactactgcgctagaggcaccggcaccggcgccatggactactggggccaaggcacaagcgtgacggtaagctccgctagcaccaagggtcctagtgtatttcccctagccccttgcagcagaagcacaagcgagagcaccgccgccctgggctgcttggtgaaggactacttccctgagcctgtcacagtgtcctggaatagcggcgccctgacaagcggcgtgcacaccttccctgccgtgctgcagagcagcggcctgtacagcctgagcagcgtggtgaccgtgccttcgtcgagcctgggcaccaagacctacacctgcaacgtggaccacaagcctagcaacaccaaggtggacaagagagtggagagcaagtacggtcctccgtgtcccccgtgccctgcccctgagttcctgggcggccctagcgtgttcctatttccacctaagcctaaggacaccctgatgatcagcagaacccctgaggtgacctgcgtggtggtggacgtgagccaagaggaccctgaggtgcagttcaactggtacgtggacggcgtggaggtgcacaacgccaagaccaagcctagagaggagcagttcaacagcacctacagagtggtgagcgtgctgaccgtgctgcaccaagactggctgaacggcaaggagtacaagtgcaaggtgagcaacaagggcctgcctagcagtatcgagaagaccatcagcaaggccaagggacagcctagagagcctcaagtgtacaccctgcctcctagccaagaggagatgaccaagaaccaagtgagcctgacctgcctggtaaaaggtttctaccctagcgacatcgccgtggagtgggagagcaacggacagcctgagaacaactacaagaccacccctcctgtgctggacagcgacggcagcttcttcctgtacagcagactgaccgtggacaagagcagatggcaagagggcaacgtgttcagctgcagcgtgatgcacgaggccctgcacaaccactacacacagaagagcctgagcctgagcctgggcaag

SEQ ID NO: 185 - DNA, 657 bp, synthetic construct
  source 1..657: mol_type = other DNA
gacgtggtgatgacacagagccctctgagcctgcctgtgaccctgggacagcctgctagcatcagctgcagaagctctcagagcctggtgcacaacaacggcatcacctacctgtactggtatcagcagagacctggacagagccctagactgctgatctacagagtgagcaacagattcagcggcgtgcctgacagattctccggctccggcagcggcaccgacttcaccctgaagatcagcagagtggaggccgaggacgtgggcgtgtactactgctttcaaggcacccatgtccctagaaccttcggccaaggcaccaagctggagatcaagagaaccgtggccgcccctagcgtgttcatcttccctcctagcgacgagcagctgaagagcggcaccgctagcgtggtgtgcctgctgaacaacttctaccctagagaggccaaggtgcagtggaaggtggacaacgccctgcagagcggcaacagccaagagagcgtgaccgagcaagacagcaaggacagcacctacagcctgagcagcaccctgaccctgagcaaggccgactacgagaagcacaaggtgtacgcctgcgaggtgacccaccaaggcctgagcagccctgtgaccaagagcttcaacagaggcgagtgc

SEQ ID NO: 186 - DNA, 657 bp, synthetic construct
  source 1..657: mol_type = other DNA
gacatcgtgatgacacagagccctctgagcctgagcgtgacccctggacagcctgctagcatcagctgcagaagctctcagagcctggtgcacaacaacggcatcacctacctgtactggtacctgcagaagcctggacagagccctcagctgctgatctacagagtgagcaacagattcagcggcgtgcctgacagattctccggctccggcagcggcaccgacttcaccctgaagatcagcagagtggaggccgaggacgtgggcgtgtactactgctttcaaggcacccatgtccctagaaccttcggccaaggcaccaaggtggagatcaagagaaccgtggccgcccctagcgtgttcatcttccctcctagcgacgagcagctgaagagcggcaccgctagcgtggtgtgcctgctgaacaacttctaccctagagaggccaaggtgcagtggaaggtggacaacgccctgcagagcggcaacagccaagagagcgtgaccgagcaagacagcaaggacagcacctacagcctgagcagcaccctgaccctgagcaaggccgactacgagaagcacaaggtgtacgcctgcgaggtgacccaccaaggcctgagcagccctgtgaccaagagcttcaacagaggcgagtgc

SEQ ID NO: 187 - DNA, 657 bp, synthetic construct
  source 1..657: mol_type = other DNA
gacatcgtgatgacacagacccctctcagcagccctgtgaccctaggacagcctgctagcatcagctgcagaagctctcagagcctggtgcacaacaacggcatcacctacctgtactggtatcagcagagacctggacagcctcctagactgctgatctacagagtgagcaacagattcagcggagtacctgacagatttagcggttccggcgccggcaccgacttcaccctgaagatcaacagagtggaggccgaggacgtgggcgtgtacttctgctttcaaggcacccatgtccctagaaccttcggccaaggcaccaagctggagatcaagagaaccgtggccgcccctagcgtgttcatcttccctcctagcgacgagcagctgaagagcggcaccgctagcgtggtgtgcctgctgaacaacttctaccctagagaggccaaggtgcagtggaaggtggacaacgccctgcagagcggcaacagccaagagagcgtgaccgagcaagacagcaaggacagcacctacagcctgagcagcaccctgaccctgagcaaggccgactacgagaagcacaaggtgtacgcctgcgaggtgacccaccaaggcctgtcaagccctgtaactaagagcttcaacagaggcgagtgc

SEQ ID NO: 188 - DNA, 657 bp, synthetic construct
  source 1..657: mol_type = other DNA
gacatcgagatgacacagagccctctgagcctgcctgtgaccctgggacagcctgctagcatcagctgcagaagctctcagagcctggtgcacaacaacggcatcacctacctgtactggtatcagcagagacctggacagagccctagaagactgatctacagagtgagcaacagattcagcggcgtgcctgacagattctccggctccggcagcggcaccgacttcaccctgaagatcagcagagtggaggccgaggacgtgggcgtgtacttctgctttcaaggcacccatgtccctagaaccttcggcggcggcaccaagctggagatcaagagaaccgtggccgcccctagcgtgttcatcttccctcctagcgacgagcagctgaagagcggcaccgctagcgtggtgtgcctgctgaacaacttctaccctagagaggccaaggtgcagtggaaggtggacaacgccctgcagagcggcaacagccaagagagcgtgaccgagcaagacagcaaggacagcacctacagcctgagcagcaccctgaccctgagcaaggccgactacgagaagcacaaggtgtacgcctgcgaggtgacccaccaaggcctgagcagccctgtgaccaagagcttcaacagaggcgagtgc

SEQ ID NO: 189 - DNA, 657 bp, synthetic construct
  source 1..657: mol_type = other DNA
gagatcgtgctgacacagagccctgcgacactgagcctgagccctggcgagagagctacgctgagctgccgcagctctcagagcctggtgcacaacaacggcatcacctacctgtactggtacctgcagaagcctggccaagcccctaagctgctgatctacagagtgagcaacagattcagcggcatccctgacagattctccggctccggccctggcaccgacttcaccctgaccatcagcagcctggagcctgaggacctggccgtgtacttctgcttccaaggcacccatgtacctagaaccttcggcggcggcaccaagctggagatcaagagaaccgtggccgcccctagcgtgttcatcttccctcctagcgacgagcagctgaagagcggcaccgctagcgtggtgtgcctgctgaacaacttctaccctagagaggccaaggtgcagtggaaggtggacaacgccctgcagagcggcaacagccaagagagcgtgaccgagcaagacagcaaggacagcacctacagcctgagcagcaccctgaccctgagcaaggccgactacgagaagcacaaggtgtacgcctgcgaggtgacccaccaaggcctgagcagccctgtgaccaagagcttcaacagaggcgagtgc

SEQ ID NO: 190 - DNA, 356 bp, synthetic construct
  source 1..356: mol_type = other DNA
caagtgcagctgcagcaaccgggcacagagctggtgaagcctggcagcagcgtgaacctgagctgcaaggctagcggcttcaccttcacaagatactggatgcactgggtgaaggagagacctggccacggcctggagtggatcggcaacatcaaccctaacaacggcggcaccgacttcaacgagaagttcaagaacaaggccaccctgaccgtgcacaagagcagcaccaccgtgttcattcagctgagcagcctgacaagcgaggacagcgccgtgtactactgcgctagaggcaccggcaccggcgccatggactactggggccaaggcacaagcgtgaccgtgtcgtcggc

SEQ ID NO: 191 - DNA, 1335 bp, synthetic construct
  source 1..1335: mol_type = other DNA
caagtgcagctgcagcaaccgggcacagagctggtgaagcctggcagcagcgtgaacctgagctgcaaggctagcggcttcaccttcacaagatactggatgcactgggtgaaggagagacctggccacggcctggagtggatcggcaacatcaaccctaacaacggcggcaccgacttcaacgagaagttcaagaacaaggccaccctgaccgtgcacaagagcagcaccaccgtgttcattcagctgagcagcctgacaagcgaggacagcgccgtgtactactgcgctagaggcaccggcaccggcgccatggactactggggccaaggcacaagcgtgaccgtgtcgtcggctagcaccaagggtccttccgtgtttcctctagccccttgcagcagaagcacaagcgagagcaccgccgccctgggctgcctggttaaagattatttccctgagcctgtgaccgtgagctggaatagcggcgccctgacaagcggcgtgcacaccttccctgccgtgctgcagagcagcggcctgtacagcctgagcagcgtggtgaccgtgccttctagcagcctgggtactaagacctacacctgcaacgtggaccacaagcctagcaacaccaaggtggacaagagagtggagagcaagtacggtcctccgtgtcccccgtgccctgcccctgagttcctgggcggccctagcgttttcttgtttccacctaagcctaaggacaccctgatgatcagcagaacccctgaggtgacctgcgtggtggtggacgtgagccaagaggaccctgaggtgcagttcaactggtacgtggacggcgtggaggtgcacaacgccaagaccaagcctagagaggagcagttcaacagcacctacagagtggtgagcgtgctgaccgtgctgcaccaagactggctgaacggcaaggagtacaagtgcaaggtgagcaacaagggcctgcctagcagtattgaaaagaccatcagcaaggccaagggacagcctagagagcctcaagtgtacaccctgcctcctagccaagaggagatgaccaagaaccaagtgagcctgacctgcctggtgaaggggttttaccctagcgacatcgccgtggagtgggagagcaacggacagcctgagaacaactacaagaccacccctcctgtgctggacagcgacggcagcttcttcctgtacagcagactgaccgtggacaagagcagatggcaagagggcaacgtgttcagctgcagcgtgatgcacgaggccctgcacaaccactacacacagaagagcctgagcctgagcctgggcaag

SEQ ID NO: 192 - DNA, 336 bp, synthetic construct
  source 1..336: mol_type = other DNA
gacgtggtgatgacacagacccctctgagcctgcctgtgagcctgggcgaccaagctagcatcagctgcagaagctctcagagcctggtgcacaacaacggcatcacctacctgtactggtacctgcagaagcctggacagagccctaagctgctgatctacagagtgagcaacagattcagcggcgtgcctgacagattcggcggcagcggcagcggcaccgacttcaccctgaagatcagcagagtggaggccgaggacctgggcgtgtacttctgcttccaaggcacgcatgtacctagaaccttcggcggcggcaccaagctggagatcaag

SEQ ID NO: 193 - DNA, 657 bp, synthetic construct
  source 1..657: mol_type = other DNA
gacgtggtgatgacacagacccctctgagcctgcctgtgagcctgggcgaccaagctagcatcagctgcagaagctctcagagcctggtgcacaacaacggcatcacctacctgtactggtacctgcagaagcctggacagagccctaagctgctgatctacagagtgagcaacagattcagcggcgtgcctgacagattcggcggcagcggcagcggcaccgacttcaccctgaagatcagcagagtggaggccgaggacctgggcgtgtacttctgcttccaaggcacgcatgtacctagaaccttcggcggcggcaccaagctggagatcaagagaaccgtggccgcccctagcgtgttcatcttccctcctagcgacgagcagctgaagagcggcaccgctagcgtggtgtgcctgctgaacaacttctaccctagagaggccaaggtgcagtggaaggtggacaacgccctgcagagcggcaacagccaagagagcgtgaccgagcaagacagcaaggacagcacctacagcctgagcagcaccctgaccctgagcaaggccgactacgagaagcacaaggtgtacgcctgcgaggtgacccaccaaggcctgagcagccctgtgaccaagagcttcaacagaggcgagtgc

SEQ ID NO: 194 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 195 - DNA, 981 bp, Homo sapiens
  source 1..981: mol_type = genomic DNA
gctagcaccaagggtccttccgtgtttcctctagccccttgcagcagaagcacaagcgagagcaccgccgccctgggctgcctggttaaagattatttccctgagcctgtgaccgtgagctggaatagcggcgccctgacaagcggcgtgcacaccttccctgccgtgctgcagagcagcggcctgtacagcctgagcagcgtggtgaccgtgccttctagcagcctgggtactaagacctacacctgcaacgtggaccacaagcctagcaacaccaaggtggacaagagagtggagagcaagtacggtcctccgtgtcccccgtgccctgcccctgagttcctgggcggccctagcgttttcttgtttccacctaagcctaaggacaccctgatgatcagcagaacccctgaggtgacctgcgtggtggtggacgtgagccaagaggaccctgaggtgcagttcaactggtacgtggacggcgtggaggtgcacaacgccaagaccaagcctagagaggagcagttcaacagcacctacagagtggtgagcgtgctgaccgtgctgcaccaagactggctgaacggcaaggagtacaagtgcaaggtgagcaacaagggcctgcctagcagtattgaaaagaccatcagcaaggccaagggacagcctagagagcctcaagtgtacaccctgcctcctagccaagaggagatgaccaagaaccaagtgagcctgacctgcctggtgaaggggttttaccctagcgacatcgccgtggagtgggagagcaacggacagcctgagaacaactacaagaccacccctcctgtgctggacagcgacggcagcttcttcctgtacagcagactgaccgtggacaagagcagatggcaagagggcaacgtgttcagctgcagcgtgatgcacgaggccctgcacaaccactacacacagaagagcctgagcctgagcctgggcaag

SEQ ID NO: 196 - DNA, 981 bp, Homo sapiens
  source 1..981: mol_type = genomic DNA
gctagcacgaaaggtccaagcgttttccctctagccccttgcagcagaagcacaagcgagagcaccgccgccctgggctgccttgtaaaagattacttccctgagcctgtgaccgtgagttggaacagcggcgccctgacaagcggcgtgcacaccttccctgccgtgctgcagagcagcggcctgtacagcctgagcagcgtggtgaccgtgccaagcagcagcctgggcaccaagacctacacctgcaacgtggaccacaagcctagcaacaccaaggtggacaagagagtggagagcaagtacggtcctccgtgtcccccgtgccctgcccctgagttcctgggcggcccttcggtgtttctgtttccacctaagcctaaggacaccctgatgatcagcagaacccctgaggtgacctgcgtggtggtggacgtgagccaagaggaccctgaggtgcagttcaactggtacgtggacggcgtggaggtgcacaacgccaagaccaagcctagagaggagcagttcaacagcacctacagagtggtgagcgtgctgaccgtgctgcaccaagactggctgaacggcaaggagtacaagtgcaaggtgagcaacaagggcctgcctagttccattgagaagaccatcagcaaggccaagggacagcctagagagcctcaagtgtacaccctgcctcctagccaagaggagatgaccaagaaccaagtgagcctgacctgcctggtgaagggattctaccctagcgacatcgccgtggagtgggagagcaacggacagcctgagaacaactacaagaccacccctcctgtgctggacagcgacggcagcttcttcctgtacagcagactgaccgtggacaagagcagatggcaagagggcaacgtgttcagctgcagcgtgatgcacgaggccctgcacaaccactacacacagaagagcctgagcctgagcctgggcaag

SEQ ID NO: 197 - DNA, 357 bp, synthetic construct
  source 1..357: mol_type = other DNA
cagattactctgaaagagtctggccctgggatattgcagtcctcccagaccctcagtctgacttgttctttctctgggttttcactgagcacttctgctatgggtgtgagttggattcgtcagccttcaggagagggtctggagtggctggcacacatttactgggatgatgacaagcgctataacccatccctgaagagccggctcacaatctccaaggatacctccagaaaccaggtattcctcaagatcaccagtgtggacactgcagatactgccacatactactgtgctcgaagaaggagggggtatggtatggactactggggtcaaggaacctcagtcaccgtctcctca

SEQ ID NO: 198 - DNA, 981 bp, Homo sapiens
  source 1..981: mol_type = genomic DNA
gctagcaccaagggtcctagtgtatttcccctagccccttgcagcagaagcacaagcgagagcaccgccgccctgggctgcttggtgaaggactacttccctgagcctgtcacagtgtcctggaatagcggcgccctgacaagcggcgtgcacaccttccctgccgtgctgcagagcagcggcctgtacagcctgagcagcgtggtgaccgtgccttcgtcgagcctgggcaccaagacctacacctgcaacgtggaccacaagcctagcaacaccaaggtggacaagagagtggagagcaagtacggtcctccgtgtcccccgtgccctgcccctgagttcctgggcggccctagcgtgttcctatttccacctaagcctaaggacaccctgatgatcagcagaacccctgaggtgacctgcgtggtggtggacgtgagccaagaggaccctgaggtgcagttcaactggtacgtggacggcgtggaggtgcacaacgccaagaccaagcctagagaggagcagttcaacagcacctacagagtggtgagcgtgctgaccgtgctgcaccaagactggctgaacggcaaggagtacaagtgcaaggtgagcaacaagggcctgcctagcagtatcgagaagaccatcagcaaggccaagggacagcctagagagcctcaagtgtacaccctgcctcctagccaagaggagatgaccaagaaccaagtgagcctgacctgcctggtaaaaggtttctaccctagcgacatcgccgtggagtgggagagcaacggacagcctgagaacaactacaagaccacccctcctgtgctggacagcgacggcagcttcttcctgtacagcagactgaccgtggacaagagcagatggcaagagggcaacgtgttcagctgcagcgtgatgcacgaggccctgcacaaccactacacacagaagagcctgagcctgagcctgggcaag

SEQ ID NO: 199 - DNA, 981 bp, Homo sapiens
  source 1..981: mol_type = genomic DNA
gctagcaccaagggtcctagtgtatttcccctagccccttgcagcagaagcacaagcgagagcaccgccgccctgggctgcttggtgaaggactacttccctgagcctgtgaccgtgagctggaacagcggcgccctgacaagcggcgtgcacaccttccctgccgtgctgcagagcagcggcctgtacagcctgagcagcgtggtgaccgtgccttcgtcgagcctgggcaccaagacctacacctgcaacgtggaccacaagcctagcaacaccaaggtggacaagagagtggagagcaagtacggtcctccgtgtcccccgtgccctgcccctgagttcctgggcggccctagcgtgttcctatttccacctaagcctaaggacaccctgatgatcagcagaacccctgaggtgacctgcgtggtggtggacgtgagccaagaggaccctgaggtgcagttcaactggtacgtggacggcgtggaggtgcacaacgccaagaccaagcctagagaggagcagttcaacagcacctacagagtggtgagcgtgctgaccgtgctgcaccaagactggctgaacggcaaggagtacaagtgcaaggtgagcaacaagggcctgcctagcagtatcgagaagaccatcagcaaggccaagggacagcctagagagcctcaagtgtacaccctgcctcctagccaagaggagatgaccaagaaccaagtgagcctgacctgcctggtaaaaggtttctaccctagcgacatcgccgtggagtgggagagcaacggacagcctgagaacaactacaagaccacccctcctgtgctggacagcgacggcagcttcttcctgtactcccgcctgacggttgacaagagcagatggcaagagggcaacgtgttcagctgcagcgtgatgcacgaggccctgcacaaccactacacacagaagagcctgagcctgagcctgggcaag

SEQ ID NO: 200 - protein, 107 aa, Homo sapiens [sequence omitted]

SEQ ID NO: 201 - DNA, 321 bp, Homo sapiens
  source 1..321: mol_type = genomic DNA
agaaccgtggccgcccctagcgtgttcatcttccctcctagcgacgagcagctgaagagcggcaccgctagcgtggtgtgcctgctgaacaacttctaccctagagaggccaaggtgcagtggaaggtggacaacgccctgcagagcggcaacagccaagagagcgtgaccgagcaagacagcaaggacagcacctacagcctgagcagcaccctgaccctgagcaaggccgactacgagaagcacaaggtgtacgcctgcgaggtgacccaccaaggcctgagcagccctgtgaccaagagcttcaacagaggcgagtgc

SEQ ID NO: 202 - DNA, 321 bp, Homo sapiens
  source 1..321: mol_type = genomic DNA
agaaccgtggccgcccctagcgtgttcatcttccctcctagcgacgagcagctgaagagcggcaccgctagcgtggtgtgcctgctgaacaacttctaccctagagaggccaaggtgcagtggaaggtggacaacgccctgcagagcggcaacagccaagagagcgtgaccgagcaagacagcaaggacagcacctacagcctgagcagcaccctgaccctgagcaaggccgactacgagaagcacaaggtgtacgcctgcgaggtgacccaccaaggcctgtcaagccctgtaactaagagcttcaacagaggcgagtgc

SEQ ID NO: 203 - protein, 19 aa, synthetic construct
MGWTLVFLFLLSVTAGVHS

SEQ ID NO: 204 - DNA, 57 bp, synthetic construct
  source 1..57: mol_type = other DNA
atgggctggaccctggtgtttctcttcctgctgagcgtgaccgccggcgtgcacagc

SEQ ID NO: 205 - protein, 20 aa, synthetic construct
MVSSAQFLGLLLLCFQGTRC

SEQ ID NO: 206 - DNA, 60 bp, synthetic construct
  source 1..60: mol_type = other DNA
atggtgagcagcgctcagttcctgggcctgctgctgctgtgttttcaaggcacaagatgt

SEQ ID NO: 207 - DNA, 60 bp, synthetic construct
  source 1..60: mol_type = other DNA
atggtgagcagcgctcagttcctgggcctgctgctgctgtgcttccaaggcacaagatgc

SEQ ID NO: 208 - DNA, 60 bp, synthetic construct
  source 1..60: mol_type = other DNA
atggtgagcagcgctcagttcctgggcctgctgctgctgtgtttccaagggacaagatgt

SEQ ID NO: 209-223 - skipped (15 entries)

SEQ ID NO: 224 - DNA, 321 bp, synthetic construct
  source 1..321: mol_type = other DNA
gacatccagatgactcagtctccagcctccctatctgcatctgtgggagaaactgtcaccatcacatgtcgagcaagtgggaatattcacaattatttagcatggtatcagcagagacagggaaaatctcctcagctcctggtctataatgcaaaaaccttaccagatggtgtgccatcaaggttcagtggcagtggatcaggaacacaatattctctcaagatcaacagcctgcagcctgaagattttgggagttattgctgtcaacatttttggagtactccgctcacgttcggtgctgggaccaagttggagctgaaa

SEQ ID NO: 225-228 - skipped (4 entries)

SEQ ID NO: 229 - DNA, 339 bp, synthetic construct
  source 1..339: mol_type = other DNA
gacattgtgatgtcacagtctccatcctccctagctgtgtcagttggagagaaggttactatgagctgcaagtccagtcagagccttttatatagtaccaatcaagagaactacttggcctggtaccagcagaaaccagggcagtctcctaaactgctgatttactgggcatcctctagggaatctggggtccctgatcgctttacaggcagtggatctgggacagatttcactctcaccatcagcagtgtgaaggctgaagacctggcagtttattactgtcagcaatattatagctatcctcggacgttcggtggaggcaccaagctggaaatcaaa

SEQ ID NO: 230-240 - skipped (11 entries)

SEQ ID NO: 241 - DNA, 336 bp, synthetic construct
  source 1..336: mol_type = other DNA
gatgttgtgatgacccaaactccactctccctgcctgtcagtcttggagatcaagcttccatctcttgcagatctagtcagagccttgtacacaacaatggaatcacctatttatattggtacctgcagaagccaggccagtctccaaagctcctgatctacagggtttccaaccgattttctggggtcccagacaggttcggtggcagtggatcagggacagatttcacactcaagatcagcagagtggaggctgaggatctgggagtttatttctgctttcaaggtacacatgttcctcggacgttcggtggaggcaccaagctggaaatcaaa

SEQ ID NO: 242 - DNA, 336 bp, synthetic construct
  source 1..336: mol_type = other DNA
gatgttgtgatgacccaaactccactctccctgcctgtcagtcttggagatcacgcttccatctcttgcagatctagtcagagccttgtacacagcaatggaatcacccatttatattggtacctgcagaggccaggccagactccaaagctcctgatctacagggtttccaaccgattttctggggtcccagacaggttcagtggcagtggatcagggacagatttcacactcaagatcagcagcgtggaggctgaggatctgggagtttatttctgctttcaaggtacacatgttcctcggacgttcggtggaggcaccaagctggaaatcgaa

SEQ ID NO: 243 - DNA, 336 bp, synthetic construct
  source 1..336: mol_type = other DNA
gatgtggtgatgacccaaactccactctccctgcctgtcagtcttggagatcaggcttccatctcttgcagatctagtcagagccttgtacacagcaatggaaacacccatttatattggtacctgcagaagccaggccagtctccaaagctcctgatctacagggtttccagccgattttctggggtcccagacaggttcagtggcagtggatcagggacagatttcacactcaagatcagcagagtggaggctgaggatctgggagtttatttctgctttcagggtacacatgttcctcggacgttcggtggaggcaccaagctggaaatcaaa

SEQ ID NO: 244-269 - skipped (26 entries)

SEQ ID NO: 270 - DNA, 321 bp, synthetic construct
  source 1..321: mol_type = other DNA
agtattgtgatgacccagactcccaaattcctgcttgtatcagcaggagacagggttaccataacctgcaaggccagtcagagtgtgagtaatgatgtagcttggtaccaacagaagccagggcagtctcctaaactgctaatatactatgcatccaatcgctgcactggagtccctgatcgcttcactggcagtggatatgggacggatttcactttcaccatcagcactgtacaggctgaagacctggcagtttatttctgtcagcaggattataggtctccgctcacgttcggtgctgggaccaagctggagctgaaa

SEQ ID NO: 271-298 - skipped (28 entries)

SEQ ID NO: 299 - protein, 7 aa, synthetic construct
DYTFTNY

SEQ ID NO: 300-303 - skipped (4 entries)

SEQ ID NO: 304 - protein, 7 aa, synthetic construct
GFTFTDY

SEQ ID NO: 305-313 - skipped (9 entries)

SEQ ID NO: 314 - protein, 7 aa, synthetic construct
GFTFTRY

SEQ ID NO: 315 - protein, 7 aa, synthetic construct
GYTFTIF

SEQ ID NO: 316 - protein, 7 aa, synthetic construct
GYTFTRF

SEQ ID NO: 317-324 - skipped (8 entries)

SEQ ID NO: 325 - protein, 9 aa, synthetic construct
GFSLSTSAM

SEQ ID NO: 326-340 - skipped (15 entries)

SEQ ID NO: 341 - protein, 6 aa, synthetic construct
NPNNGG

SEQ ID NO: 342 - skipped

SEQ ID NO: 343 - protein, 6 aa, synthetic construct
DPNSGG

SEQ ID NO: 344-346 - skipped (3 entries)

SEQ ID NO: 347 - protein, 8 aa, synthetic construct
RNKAKGFT

SEQ ID NO: 348-361 - skipped (14 entries)

SEQ ID NO: 362 - protein, 5 aa, synthetic construct
YWDDD

SEQ ID NO: 363-394 - skipped (32 entries)

SEQ ID NO: 395 - protein, 4 aa, synthetic construct
DFDV

SEQ ID NO: 396-399 - skipped (4 entries)

SEQ ID NO: 400 - protein, 4 aa, synthetic construct
DINY

SEQ ID NO: 401-409 - skipped (9 entries)

SEQ ID NO: 410 - protein, 9 aa, synthetic construct
GTGTGAMDY

SEQ ID NO: 411-434 - skipped (24 entries)

SEQ ID NO: 435 - protein, 9 aa, synthetic construct
RRRGYGMDY

SEQ ID NO: 436-459 - skipped (24 entries)

SEQ ID NO: 460 - protein, 11 aa, synthetic construct
RASGNIHNYLA

SEQ ID NO: 461-463 - skipped (3 entries)

SEQ ID NO: 464 - protein, 17 aa, synthetic construct
KSSQSLLYSTNQENYLA

SEQ ID NO: 465-472 - skipped (8 entries)

SEQ ID NO: 473 - protein, 11 aa, synthetic construct
QSLVHNNGITY

SEQ ID NO: 474 - protein, 16 aa, synthetic construct
RSSQSLVHSNGITHLY

SEQ ID NO: 475 - protein, 16 aa, synthetic construct
RSSQSLVHSNGNTHLY

SEQ ID NO: 476-494 - skipped (19 entries)

SEQ ID NO: 495 - protein, 11 aa, synthetic construct
KASQSVSNDVA

SEQ ID NO: 496-517 - skipped (22 entries)

SEQ ID NO: 518 - protein, 7 aa, synthetic construct
NAKTLPD

SEQ ID NO: 519-522 - skipped (4 entries)

SEQ ID NO: 523 - protein, 7 aa, synthetic construct
WASSRES

SEQ ID NO: 524-528 - skipped (5 entries)

SEQ ID NO: 529 - protein, 7 aa, synthetic construct
RVSNRFS

SEQ ID NO: 530 - protein, 7 aa, synthetic construct
RVSSRFS

SEQ ID NO: 531-539 - skipped (9 entries)

SEQ ID NO: 540 - protein, 7 aa, synthetic construct
YASNRCT

SEQ ID NO: 541-556 - skipped (16 entries)

SEQ ID NO: 557 - protein, 9 aa, synthetic construct
QHFWSTPLT

SEQ ID NO: 558-561 - skipped (4 entries)

SEQ ID NO: 562 - protein, 9 aa, synthetic construct
QQYYSYPRT

SEQ ID NO: 563-570 - skipped (8 entries)

SEQ ID NO: 571 - protein, 9 aa, synthetic construct
FQGTHVPRT

SEQ ID NO: 572-586 - skipped (15 entries)

SEQ ID NO: 587 - protein, 9 aa, synthetic construct
QQDYRSPLT

SEQ ID NO: 588-600 - skipped (13 entries)

SEQ ID NO: 601 - protein, 5 aa, synthetic construct
GGGGS

SEQ ID NO: 602 - protein, 10 aa, synthetic construct
GGGGSGGGGS

SEQ ID NO: 603 - protein, 20 aa, synthetic construct
GGGGSGGGGSGGGGSGGGGS

SEQ ID NO: 604 - protein, 25 aa, synthetic construct
GGGGSGGGGSGGGGSGGGGSGGGGS

SEQ ID NO: 605 - protein, 30 aa, synthetic construct
GGGGSGGGGSGGGGSGGGGSGGGGSGGGGS

SEQ ID NO: 606 - skipped

SEQ ID NO: 607 - protein, 40 aa, synthetic construct
GGGGSGGGGSGGGGSGGGGSGGGGSGGGGSGGGGSGGGGS

SEQ ID NO: 608 - protein, 5 aa, synthetic construct
GGGGS

SEQ ID NO: 609-799 - skipped (191 entries)

SEQ ID NO: 800 - protein, 7 aa, synthetic construct
NGAVHLY

SEQ ID NO: 801 - protein, 7 aa, synthetic construct
TLAVVSL

SEQ ID NO: 802 - protein, 40 aa, synthetic construct; identical to SEQ ID NO: 607
  VARIANT 1..40: note = This sequence may encompass 0-8 GGGGS repeating units

SEQ ID NO: 803 - protein, 743 aa, unidentified
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 804 - protein, 736 aa, Adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 805 - DNA, 969 bp, Mus sp.
  source 1..969: mol_type = genomic DNA
gccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggt

SEQ ID NO: 806-919 - skipped (114 entries)

SEQ ID NO: 920 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 921 - protein, 20 aa, synthetic construct
  SITE 8: note = Phosphorylated residue
  SITE 13: note = Phosphorylated residue
TPGSRSRTPSLPTPPTREPK

SEQ ID NO: 922 - protein, 19 aa, synthetic construct
  SITE 9: note = Phosphorylated residue
  SITE 11: note = Phosphorylated residue
  SITE 14: note = Phosphorylated residue
GTPGSRSRTPSLPTPPTRE

SEQ ID NO: 923 - protein, 37 aa, synthetic construct
  SITE 18: note = Phosphorylated residue
  SITE 26: note = Phosphorylated residue
  SITE 31: note = Phosphorylated residue
RENAKAKTDHGAEIVYKSPVVSGDTSPRHLSNVSSTG

SEQ ID NO: 924 - protein, 8 aa, synthetic construct
PTREPKKV

SEQ ID NO: 925 - protein, 7 aa, synthetic construct
ARMVSKS

SEQ ID NO: 926 - protein, 24 aa, synthetic construct
SPSSAKSRLQTAPVPMPDLKNVKS

SEQ ID NO: 927-1028 - skipped (102 entries)

SEQ ID NO: 1029 - DNA, 23 bp, unidentified
  source 1..23: mol_type = unassigned DNA
acaaacaccattgtcacactcca

SEQ ID NO: 1030 - DNA, 71 bp, unidentified
  source 1..71: mol_type = unassigned DNA
acaaacaccattgtcacactccacacaaacaccattgtcacactccacacaaacaccattgtcacactcca

SEQ ID NO: 1031 - DNA, 23 bp, unidentified
  source 1..23: mol_type = unassigned DNA
tccataaagtaggaaacactaca

SEQ ID NO: 1032 - DNA, 22 bp, unidentified
  source 1..22: mol_type = unassigned DNA
agtgaattctaccagtgccata

SEQ ID NO: 1033 - DNA, 24 bp, unidentified
  source 1..24: mol_type = unassigned DNA
agtgtgagttctaccattgccaaa

SEQ ID NO: 1034 - DNA, 23 bp, unidentified
  source 1..23: mol_type = unassigned DNA
agcaaaaatgtgctagtgccaaa

SEQ ID NO: 1035 - DNA, 130 bp, unidentified
  source 1..130: mol_type = unassigned DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 1036 - DNA, 141 bp, unidentified
  source 1..141: mol_type = unassigned DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 1037 - DNA, 130 bp, unidentified
  source 1..130: mol_type = unassigned DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 1038 - DNA, 141 bp, unidentified
  source 1..141: mol_type = unassigned DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 1039 - DNA, 1715 bp, synthetic construct
  source 1..1715: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattc

SEQ ID NO: 1040 - DNA, 299 bp, synthetic construct
  source 1..299: mol_type = other DNA
tagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatct

SEQ ID NO: 1041 - DNA, 283 bp, synthetic construct
  source 1..283: mol_type = other DNA
catggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 1042 - DNA, 260 bp, synthetic construct
  source 1..260: mol_type = other DNA
ccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcggg

SEQ ID NO: 1043 - DNA, 654 bp, unidentified
  source 1..654: mol_type = unassigned DNA
cgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagc

SEQ ID NO: 1044 - DNA, 699 bp, unidentified
  source 1..699: mol_type = unassigned DNA
gatctaacatatcctggtgtggagtagcggacgctgctatgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgcggggatcctctag

SEQ ID NO: 1045 - DNA, 557 bp, unidentified
  source 1..557: mol_type = unassigned DNA
tagtatctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcagcc

SEQ ID NO: 1046 - DNA, 382 bp, synthetic construct
  source 1..382: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattac

SEQ ID NO: 1047 - DNA, 1736 bp, synthetic construct
  source 1..1736: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattc

SEQ ID NO: 1048 - DNA, 365 bp, unidentified
  source 1..365: mol_type = unassigned DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgta

SEQ ID NO: 1049 - DNA, 1714 bp, synthetic construct
  source 1..1714: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

SEQ ID NO: 1050 - DNA, 380 bp, synthetic construct
  source 1..380: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 1051 - DNA, 134 bp, synthetic construct
  source 1..134: mol_type = other DNA
tcagatcgcctggagacgccatccacgctgttttgacctccatagaagacaccgggaccgatccagcctccgcggattcgaatcccggccgggaacggtgcattggaacgcggattccccgtgccaagagtgac

SEQ ID NO: 1052 - DNA, 102 bp, synthetic construct
  source 1..102: mol_type = other DNA
ttgacctccatagaagacaccgggaccgatccagcctccgcggattcgaatcccggccgggaacggtgcattggaacgcggattccccgtgccaagagtgac

SEQ ID NO: 1053 - DNA, 59 bp, synthetic construct
  source 1..59: mol_type = other DNA
attcgaatcccggccgggaacggtgcattggaacgcggattccccgtgccaagagtgac

SEQ ID NO: 1054 - DNA, 53 bp, Homo sapiens
  source 1..53: mol_type = genomic DNA
ctcctgggcaacgtgctggtctgtgtgctggcccatcactttggcaaagaatt

SEQ ID NO: 1055 - DNA, 54 bp, unidentified
  source 1..54: mol_type = unassigned DNA
ctcctgggcaacgtgctggttattgtgctgtctcatcattttggcaaagaattc

SEQ ID NO: 1056 - DNA, 32 bp, synthetic construct
  source 1..32: mol_type = other DNA
gtaagtaccgcctatagagtctataggcccac

SEQ ID NO: 1057 - DNA, 15 bp, synthetic construct
  source 1..15: mol_type = other DNA
gtaagtaccgcctat

SEQ ID NO: 1058 - DNA, 347 bp, synthetic construct
  source 1..347: mol_type = other DNA
aaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 1059 - DNA, 168 bp, synthetic construct
  source 1..168: mol_type = other DNA
tctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 1060 - DNA, 73 bp, synthetic construct
  source 1..73: mol_type = other DNA
aaaaaatgctttcttcttttaatatacttttcttttgctaatcatgttcatacctcttatcttcctcccacag

SEQ ID NO: 1061 - DNA, 73 bp, synthetic construct
  source 1..73: mol_type = other DNA
aggctggattattctgagtccaagctaggcccttttgctaatcatgttcatacctcttatcttcctcccacag

SEQ ID NO: 1062 - DNA, 73 bp, synthetic construct
  source 1..73: mol_type = other DNA
aaaaaatgctttcttcttttcaagctaggcccttttgctaatcatgttcatacctcttatcttcctcccacag

SEQ ID NO: 1063 - DNA, 53 bp, synthetic construct
  source 1..53: mol_type = other DNA
caagctaggcccttttgctaatcatgttcatacctcttatcttcctcccacag

SEQ ID NO: 1064 - DNA, 172 bp, Simian virus 40
  source 1..172: mol_type = genomic DNA
gaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtaccc

SEQ ID NO: 1065 - DNA, 1074 bp, unidentified
  source 1..1074: mol_type = unassigned DNA
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgag

SEQ ID NO: 1066 - DNA, 41 bp, unidentified
  source 1..41: mol_type = unassigned DNA
cctctgctaaccatgttcatgccttcttctttttcctacag

SEQ ID NO: 1067 - DNA, 566 bp, synthetic construct
  source 1..566: mol_type = other DNA
tcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaatt

SEQ ID NO: 1068 - DNA, 491 bp, synthetic construct
  source 1..491: mol_type = other DNA
attcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaatt

SEQ ID NO: 1069 - DNA, 387 bp, synthetic construct
  source 1..387: mol_type = other DNA
tcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccactctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaatt

SEQ ID NO: 1070 - DNA, 292 bp, synthetic construct
  source 1..292: mol_type = other DNA
tcagatcgcctggagacgccatccacgctgttttgacctccatagaagacaccgggaccgatccaggtaagtaccgcctatagagtctataggcccacaaaaaatgctttcttcttttaatatacttttttgtttatcttatttctaatactttccctaatctctttcgctggattattctgagtccaagctaggcccttttgctaatcatgttcatacctcttatcttcctcccacagctcctgggcaacgtgctggtctgtgtgctggcccatcactttggcaaagaatt

SEQ ID NO: 1071 - DNA, 84 bp, synthetic construct
  source 1..84: mol_type = other DNA
atggcgacgggttcaagaacttccctacttcttgcatttggcctgctttgtttgccgtggttacaggagggctcggcagctgcc

SEQ ID NO: 1072 - DNA, 93 bp, synthetic construct
  source 1..93: mol_type = other DNA
gccgccaccatggcgacgggttcaagaacttccctacttcttgcatttggcctgctttgtttgccgtggttacaggagggctcggcagctgcc

SEQ ID NO: 1073 - DNA, 96 bp, synthetic construct
  source 1..96: mol_type = other DNA
gccgccaccatgggggtgcacgaatgtcctgcctggctgtggcttctcctgtccctgctgtcgctccctctgggcctcccagtcctgggcgctgcc

SEQ ID NO: 1074 - DNA, 66 bp, synthetic construct
  source 1..66: mol_type = other DNA
gccgccaccatgggagtcaaagttctgtttgccctgatctgcatcgctgtggccgaggccgctgcc

SEQ ID NO: 1075 - DNA, 72 bp, synthetic construct
  source 1..72: mol_type = other DNA
gccgccaccatgaaacacctgtggttcttcctcctgctggtggcagctcccagatgggtcctgtccgctgcc

SEQ ID NO: 1076 - DNA, 93 bp, synthetic construct
  source 1..93: mol_type = other DNA
gccgccaccatggacctcctgcacaagaacatgaaacacctgtggttcttcctcctcctggtggcagctcccagatgggtgctgtccgctgcc

SEQ ID NO: 1077 - DNA, 69 bp, synthetic construct
  source 1..69: mol_type = other DNA
gccgccaccatggctttcctctggctcctctcctgctgggccctcctgggtaccaccttcggcgctgcc

SEQ ID NO: 1078 - DNA, 81 bp, synthetic construct
  source 1..81: mol_type = other DNA
gccgccaccatgccccccaagaagtgcctgctgctgctgctgaccctgctgctgctgatctccaccaccttcggcgctgcc

SEQ ID NO: 1079 - DNA, 12 bp, synthetic construct
  source 1..12: mol_type = other DNA
gccgccaccatg

SEQ ID NO: 1080 - DNA, 81 bp, synthetic construct
  source 1..81: mol_type = other DNA
gccgccaccatggacatgagggtccctgctcagctcctggggctcctgctgctctggctctcaggtgccagatgtgctgcc

SEQ ID NO: 1081 - DNA, 66 bp, synthetic construct
  source 1..66: mol_type = other DNA
gccgccaccatggaaatcaaggtgctgtttgccctcatctgtattgctgttgctgaggcagctgcc

SEQ ID NO: 1082 - DNA, 78 bp, synthetic construct
  source 1..78: mol_type = other DNA
gccgccaccatggagacagacacactcctgctatgggtactgctgctctgggttccaggttccactggtgacgctgcc

SEQ ID NO: 1083 - DNA, 57 bp, synthetic construct
  source 1..57: mol_type = other DNA
atgaacttcgggctcagcttgattttccttgtccttgttttaaaaggtgtccagtgt

SEQ ID NO: 1084 - DNA, 57 bp, synthetic construct
  source 1..57: mol_type = other DNA
atgggatggagctggatctttctctttctcctgtcaggaactacaggtgtcctctct

SEQ ID NO: 1085 - DNA, 57 bp, synthetic construct
  source 1..57: mol_type = other DNA
atgaagttgcctgttaggctgttggtgctgatgttctggattcctgcttccagcagt

SEQ ID NO: 1086 - DNA, 411 bp, synthetic construct
  source 1..411: mol_type = other DNA
atgaacttcgggctcagcttgattttccttgtccttgttttaaaaggtgtccagtgtgaggtgaagctggtggagagcggcggcgacctggtgaagcctggcggcagcctgaagctgagctgcgccgccagcggcttcaccttcagcagctacgccatgagctgggtgagacagaaccctgagaagagactggagtgggtggccagcatcagcaagggcggcaacacctactaccctaacagcgtgaagggcagattcaccatcagcagagacaacgccagaaacatcctgtacctgcagatgagcagcctgagaagcgaggacaccgccctgtactactgcgccagaggctggggcgactacggctggttcgcctactggggccaggtgaccctggtgaccgtgagcgcc

SEQ ID NO: 1087-1088 - skipped (2 entries)

SEQ ID NO: 1089 - DNA, 11 bp, synthetic construct
  source 1..11: mol_type = other DNA
gaggagccacc

SEQ ID NO: 1090-1133 - skipped (44 entries)

SEQ ID NO: 1134 - DNA, 127 bp, synthetic construct
  source 1..127: mol_type = other DNA
gatctttttccctctgccaaaaattatggggacatcatgaagccccttgagcatctgacttctggctaataaaggaaatttattttcattgcaatagtgtgttggaattttttgtgtctctcactcg

SEQ ID NO: 1135 - DNA, 477 bp, synthetic construct
  source 1..477: mol_type = other DNA
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 1136 - DNA, 552 bp, synthetic construct
  source 1..552: mol_type = other DNA
gaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagctt

SEQ ID NO: 1137-1139 - skipped (3 entries)

SEQ ID NO: 1140 - protein, 5 aa, synthetic construct
NYWMH

SEQ ID NO: 1141 - protein, 17 aa, synthetic construct
RIDPNSGGTRYNEKFKN

SEQ ID NO: 1142 - protein, 5 aa, synthetic construct
DYYMS

SEQ ID NO: 1143 - protein, 19 aa, synthetic construct
LIRNKAKGFTTEYSASVKG

SEQ ID NO: 1144 - protein, 5 aa, synthetic construct
RYWMH

SEQ ID NO: 1145 - protein, 17 aa, synthetic construct
NINPNNGGTDFNEKFKN

SEQ ID NO: 1146 - protein, 16 aa, synthetic construct
RSSQSLVHNNGITYLY

SEQ ID NO: 1147 - protein, 5 aa, synthetic construct
IFWMH

SEQ ID NO: 1148 - protein, 17 aa, synthetic construct
KINPNNGGGDYNEKFKS

SEQ ID NO: 1149 - protein, 5 aa, synthetic construct
RFWMH

SEQ ID NO: 1150 - protein, 17 aa, synthetic construct
NINPNNGGTDNNERFKS

SEQ ID NO: 1151 - protein, 7 aa, synthetic construct
TLAVPFK

SEQ ID NO: 1152 - protein, 7 aa, synthetic construct
TSAMGVS

SEQ ID NO: 1153 - protein, 16 aa, synthetic construct
HIYWDDDKRYNPSLKS

SEQ ID NO: 1154 - protein, 16 aa, synthetic construct
RSSQSLVHNNGITYLY

SEQ ID NO: 1155 - protein, 8 aa, synthetic construct
DYTFTNYW

SEQ ID NO: 1156 - protein, 8 aa, synthetic construct
IDPNSGGT

SEQ ID NO: 1157 - protein, 6 aa, synthetic construct
AGDFDV

SEQ ID NO: 1158 - protein, 6 aa, synthetic construct
GNIHNY

SEQ ID NO: 1159 - skipped

SEQ ID NO: 1160 - protein, 8 aa, synthetic construct
GFTFTDYY

SEQ ID NO: 1161 - protein, 10 aa, synthetic construct
IRNKAKGFTT

SEQ ID NO: 1162 - protein, 6 aa, synthetic construct
VRDINY

SEQ ID NO: 1163 - protein, 12 aa, synthetic construct
QSLLYSTNQENY

SEQ ID NO: 1164 - skipped

SEQ ID NO: 1165 - protein, 8 aa, synthetic construct
GFTFTRYW

SEQ ID NO: 1166 - protein, 8 aa, synthetic construct
INPNNGGT

SEQ ID NO: 1167 - protein, 11 aa, synthetic construct
ARGTGTGAMDY

SEQ ID NO: 1168 - protein, 8 aa, synthetic construct
GYTFTIFW

SEQ ID NO: 1169 - protein, 8 aa, synthetic construct
INPNNGGG

SEQ ID NO: 1170 - protein, 11 aa, synthetic construct
QSLVHSNGITH

SEQ ID NO: 1171 - protein, 8 aa, synthetic construct
GYTFTRFW

SEQ ID NO: 1172 - protein, 11 aa, synthetic construct
QSLVHSNGNTH

SEQ ID NO: 1173 - protein, 10 aa, synthetic construct
GFSLSTSAMG

SEQ ID NO: 1174 - protein, 7 aa, synthetic construct
IYWDDDK

SEQ ID NO: 1175 - protein, 11 aa, synthetic construct
ARRRRGYGMDY

SEQ ID NO: 1176 - protein, 6 aa, synthetic construct
QSVSND

SEQ ID NO: 1177-1179 - skipped (3 entries)

SEQ ID NO: 1180 - protein, 7 aa, synthetic construct
  VARIANT 2: note = F or Y
  VARIANT 6: note = R or I
  VARIANT 7: note = Y or F
GXTFTXX

SEQ ID NO: 1181 - protein, 16 aa, synthetic construct
  VARIANT 9: note = N or S
  VARIANT 12: note = I or N
  VARIANT 14: note = Y or H
RSSQSLVHXNGXTXLY

SEQ ID NO: 1182 - protein, 7 aa, synthetic construct
  VARIANT 4: note = N or S
RVSXRFS

SEQ ID NO: 1183 - skipped

SEQ ID NO: 1184 - protein, 17 aa, synthetic construct
  VARIANT 1: note = N or K
  VARIANT 9: note = T or G
  VARIANT 11: note = F, Y or N
  VARIANT 14: note = K or R
  VARIANT 17: note = N or S
XINPNNGGXDXNEXFKX

SEQ ID NO: 1185 - protein, 16 aa, synthetic construct
  VARIANT 9: note = N or S
  VARIANT 12: note = I or N
  VARIANT 14: note = Y or H
RSSQSLVHXNGXTXLY

SEQ ID NO: 1186 - protein, 8 aa, synthetic construct
  VARIANT 2: note = F or Y
  VARIANT 6: note = R or I
  VARIANT 7: note = Y or F
GXTFTXXW

SEQ ID NO: 1187 - protein, 8 aa, synthetic construct
  VARIANT 8: note = T or G
INPNNGGX

SEQ ID NO: 1188 - protein, 11 aa, synthetic construct
  VARIANT 6: note = N or S
  VARIANT 9: note = I or N
  VARIANT 11: note = Y or H
QSLVHXNGXTX

SEQ ID NO: 1189 - protein, 324 aa, Mus sp.
AKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 1190 - protein, 393 aa, Mus sp.
AKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGLQLDETCAEAQDGELDGLWTTITIFISLFLLSVCYSAAVTLFKVKWIFSSVVELKQTLVPEYKNMIGQAP

SEQ ID NO: 1191 - protein, 330 aa, Mus sp.
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 1192 - DNA, 990 bp, Mus sp.
  source 1..990: mol_type = genomic DNA
gctaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 1193 - protein, 335 aa, Mus sp.
AKTTAPSVYPLVPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPHQRVPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEVLHNHLTTKTISRSLGK

SEQ ID NO: 1194 - protein, 404 aa, Mus sp.
KTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGLDLDDICAEAKDGELDGLWTTITIFISLFLLSVCYSASVTLFKVKWIFSSVVELKQKISPDYRNMIGQGA

SEQ ID NO: 1195 - protein, 403 aa, Mus sp.
KTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGLDLDDVCTEAQDGELDGLWTTITIFISLFLLSVCYSASVTLFKVKWIFSSVVELKQKISPDYRNMIGQGA

SEQ ID NO: 1196 - protein, 398 aa, Mus sp.
TTTAPSVYPLVPGCSDTSGSSVTLGCLVKGYFPEPVTVKWNYGALSSGVRTVSSVLQSGFYSLSSLVTVPSSTWPSQTVICNVAHPASKTELIKRIEPRIPKPSTPPGSSCPPGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPELELNETCAEAQDGELDGLWTTITIFISLFLLSVCYSASVTLFKVKWIFSSVVQVKQTAIPDYRNMIGQGA

SEQ ID NO: 1197 - protein, 106 aa, Mus sp.
ADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 1198 - DNA, 318 bp, Mus sp.
  source 1..318: mol_type = genomic DNA
gcagatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 1199 - protein, 105 aa, Mus sp.
QPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 1200 - protein, 104 aa, Mus sp.
QPKSTPTLTVFPPSSEELKENKATLVCLISNFSPSGVTVAWKANGTPITQGVDTSNPTKEGNKFMASSFLHLTSDQWRSHNSFTCQVTHEGDTVEKSLSPAECL

SEQ ID NO: 1201 - protein, 104 aa, Mus sp.
QPKSTPTLTMFPPSPEELQENKATLVCLISNFSPSGVTVAWKANGTPITQGVDTSNPTKEDNKYMASSFLHLTSDQWRSHNSFTCQVTHEGDTVEKSLSPAECL

SEQ ID NO: 1202 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1203 - DNA, 987 bp, Homo sapiens
  source 1..987: mol_type = genomic DNA
gccagcacaaaagggcccagtgtgttcccgctcgcaccaagcagcaaatcaacgtcaggcggcacagccgcgttgggttgccttgtaaaagactacttcccagaaccagttacggtgtcatggaacagtggtgcactcacgagcggcgttcataccttccccgccgtacttcagagttcaggtctttactcactttccagcgtggtcacagtaccgtcaagctctcttggaacacagacatatatctgtaacgtaaatcataagcctagcaataccaaagtcgataaacgagtggaacccaagagttgtgataaaacccacacctgtcccccttgcccggcaccggaactgctgggtggtccatcagtattcttgtttccgcctaagccaaaggacacactgatgatatccagaactccagaggttacgtgcgtagtcgtggacgtcagtcatgaagaccccgaagttaagttcaactggtacgtggatggtgtggaagtacataatgcgaagacgaaacccagggaagaacaatataactcaacttatagggtagtcagcgtcttgactgtacttcaccaagattggttgaatggcaaagagtacaaatgcaaggtaagcaacaaagcattgcctgcgccaatcgaaaagactatctcaaaagcaaagggccagccacgcgaaccacaagtgtatacattgccgcccagtcgggaagaaatgacgaaaaatcaagtcagtctcacatgcctcgtgaaaggattttatccctctgacatagctgtggagtgggaaagtaatggccaacccgaaaataattacaaaacgacgcctcccgttttggactcagatgggagttttttcctttacagtaagctgacggttgacaaaagcaggtggcaacaagggaacgtcttttcttgtagtgtgatgcatgaggcgctccacaatcattacactcaaaaatccttgagcctgtctccaggc

SEQ ID NO: 1204 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 1205 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 1206 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1207 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 1208 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 1209 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 1210 - protein, 106 aa, Homo sapiens
GQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECS

SEQ ID NO: 1211 - protein, 106 aa, Homo sapiens
GQPKAAPSVTLFPPSSEELQANKATLVCLVSDFNPGAVTVAWKADGSPVKVGVETTKPSKQSNNKYAASSYLSLTPEQWKSHRSYSCRVTHEGSTVEKTVAPAECS

SEQ ID NO: 1212 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1213 - DNA, 318 bp, Homo sapiens
  source 1..318: mol_type = genomic DNA
actgtcgcagcaccttctgtcttcatctttccgccaagcgatgaacagttgaaatctggaacagcgtccgtggtgtgcctgctcaacaacttctatcctcgggaagcgaaggtgcaatggaaggtagataatgctcttcagagtggcaattcccaagagtcagttacggagcaagatagcaaggacagcacgtattccctgtctagtacgttgactctttccaaggctgactatgaaaagcacaaggtgtatgcctgtgaagtaacccaccaaggtctctcaagtcctgtaactaagagctttaatcgaggagaatgc

SEQ ID NO: 1214-1261 - skipped (48 entries)

SEQ ID NO: 1262 - protein, 7 aa, synthetic construct
TLAVPFK

SEQ ID NO: 1263-1723 - skipped (461 entries)

SEQ ID NO: 1724 - DNA, 12 bp, synthetic construct
  source 1..12: mol_type = other DNA
agaaagaggcga

SEQ ID NO: 1725 - DNA, 12 bp, synthetic construct
  source 1..12: mol_type = other DNA
cgggccaagcgg

SEQ ID NO: 1726 - DNA, 54 bp, synthetic construct
  source 1..54: mol_type = other DNA
gagggcagaggaagtcttctaacatgcggtgacgtggaggagaatcccggccct

SEQ ID NO: 1727 - DNA, 75 bp, synthetic construct
  source 1..75: mol_type = other DNA
ggaagcggagtgaaacagactttgaattttgaccttctcaagttggcgggagacgtggagtccaaccctggacct

SEQ ID NO: 1728 - DNA, 66 bp, synthetic construct
  source 1..66: mol_type = other DNA
ggaagcggagctactaacttcagcctgctgaagcaggctggagacgtggaggagaaccctggacct

SEQ ID NO: 1729 - DNA, 18 bp, synthetic construct
  source 1..18: mol_type = other DNA
tccggaggcggcggcagc

SEQ ID NO: 1730 - DNA, 45 bp, synthetic construct
  source 1..45: mol_type = other DNA
ggtggtggtggtagcggcggcggcggctctggtggtggtggatcc

SEQ ID NO: 1731 - DNA, 75 bp, synthetic construct
  source 1..75: mol_type = other DNA
ggcggaggtggctccggaggcggaggcagcggcggaggtgggtctggcggaggcgggtgcggcggaggtggctcc

SEQ ID NO: 1732 - DNA, 609 bp, synthetic construct
  source 1..609: mol_type = other DNA
aattccgcccctctcccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaacc

SEQ ID NO: 1733 - DNA, 623 bp, synthetic construct
  source 1..623: mol_type = other DNA
taacgaattccgcccctctcccccccccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccgccgccacc

SEQ ID NO: 1734 - DNA, 54 bp, synthetic construct
  source 1..54: mol_type = other DNA
gtggagaggaagtgctgcgtggagtgcccaccatgccctgcccctcctgtggcc

SEQ ID NO: 1735 - DNA, 108 bp, synthetic construct
  source 1..108: mol_type = other DNA
gagctcaaaaccccacttggtgacacaactcacacatgcccacggtgcccagagcccaaatcttgtgacacacctcccccgtgcccacggtgcccagcacctgaactc

SEQ ID NO: 1736 - DNA, 153 bp, synthetic construct
  source 1..153: mol_type = other DNA
gagctcaaaaccccacttggtgacacaactcacacatgcccacggtgcccagagcccaaatcttgtgacacacctcccccgtgcccacggtgcccagagcccaaatcttgtgacacacctcccccatgcccacggtgcccagcacctgaactc

SEQ ID NO: 1737 - DNA, 198 bp, synthetic construct
  source 1..198: mol_type = other DNA
gagctcaaaaccccacttggtgacacaactcacacatgcccacggtgcccagagcccaaatcttgtgacacacctcccccgtgcccacggtgcccagagcccaaatcttgtgacacacctcccccatgcccacggtgcccagagcccaaatcttgtgacacacctcccccgtgcccaaggtgcccagcacctgaactc

SEQ ID NO: 1738 - DNA, 45 bp, synthetic construct
  source 1..45: mol_type = other DNA
gtgcccagggattgtggttgtaagccttgcatatgtacagtccca

SEQ ID NO: 1739 - DNA, 18 bp, synthetic construct
  source 1..18: mol_type = other DNA
gtgcccagggattgtggt

SEQ ID NO: 1740-2243 - skipped (504 entries)

SEQ ID NO: 2244 - DNA, 198 bp, synthetic construct
  source 1..198: mol_type = other DNA
gagctcaaaaccccacttggtgacacaactcacacatgcccacggtgcccagagcccaaatcttgtgacacacctcccccgtgcccacggtgcccagagcccaaatcttgtgacacaactcacacatgcccacggtgcccagagcccaaatcttgtgacacacctcccccgtgcccaaggtgcccagcacctgaactc

SEQ ID NO: 2245 - DNA, 15 bp, synthetic construct
  source 1..15: mol_type = other DNA
ggtggtggtggatcc

SEQ ID NO: 2246 - DNA, 30 bp, synthetic construct
  source 1..30: mol_type = other DNA
ggtggtggtggatccggtggtggtggatcc

SEQ ID NO: 2247 - DNA, 45 bp, synthetic construct
  source 1..45: mol_type = other DNA
ggtggtggtggatccggtggtggtggatccggtggtggtggatcc

SEQ ID NO: 2248 - DNA, 60 bp, synthetic construct
  source 1..60: mol_type = other DNA
ggtggtggtggatccggtggtggtggatccggtggtggtggatccggtggtggtggatcc

SEQ ID NO: 2249 - DNA, 75 bp, synthetic construct
  source 1..75: mol_type = other DNA
ggtggtggtggtagcggcggcggcggctctggtggtggtggatccggtggtggtggatccggtggtggtggatcc

SEQ ID NO: 2250 - DNA, 75 bp, synthetic construct
  source 1..75: mol_type = other DNA
ggtggtggtggatccggtggtggtggatccggtggtggtggatccggtggtggtggatccggtggtggtggatcc

SEQ ID NO: 2251 - DNA, 90 bp, synthetic construct
  source 1..90: mol_type = other DNA
ggtggtggtggtagcggcggcggcggctctggtggtggtggatccggtggtggtggatccggtggtggtggatccggtggtggtggatcc

SEQ ID NO: 2252 - DNA, 120 bp, synthetic construct
  source 1..120: mol_type = other DNA
ggtggtggtggtagcggcggcggcggctctggtggtggtggatccggtggtggtggatccggtggtggtggatccggtggtggtggatccggtggtggtggatccggtggtggtggatcc

SEQ ID NO: 2253 - DNA, 120 bp, synthetic construct
  source 1..120: mol_type = other DNA
ggtggtggtggatccggtggtggtggatccggtggtggtggatccggtggtggtggatccggtggtggtggatccggtggtggtggatccggtggtggtggatccggtggtggtggatcc

SEQ ID NO: 2254 - DNA, 60 bp, synthetic construct
  source 1..60: mol_type = other DNA
ggtggtggtggtagcggcggcggcggctctggtggtggtggatccggtggtggtggatcc

SEQ ID NO: 2255-2258 - skipped (4 entries)

SEQ ID NO: 2259 - DNA, 90 bp, synthetic construct
  source 1..90: mol_type = other DNA
ggtggtggtggatccggtggtggtggatccggtggtggtggatccggtggtggtggatccggtggtggtggatccggtggtggtggatcc

SEQ ID NO: 2260-3622 - skipped (1363 entries)

SEQ ID NO: 3623 - DNA, 2232 bp, synthetic construct
  source 1..2232: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtccgcttaatggtgccgtccatctttatgctcaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgccgatcctccaacggccttcaacaaggacaagctgaactctttcatcacccagtattctactggccaagtcagcgtggagatcgagtgggagctgcagaaggaaaacagcaagcggtggaacccggagatccagtacacttccaactattacaagtctaataatgttgaatttgctgttaatactgaaggtgtatatagtgaaccccgccccattggcaccagatacctgactcgtaatctgtaa

SEQ ID NO: 3624 - DNA, 2232 bp, synthetic construct
  source 1..2232: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcacaggctcgtgattctccgaagggttggcaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgccgatcctccaacggccttcaacaaggacaagctgaactctttcatcacccagtattctactggccaagtcagcgtggagatcgagtgggagctgcagaaggaaaacagcaagcggtggaacccggagatccagtacacttccaactattacaagtctaataatgttgaatttgctgttaatactgaaggtgtatatagtgaaccccgccccattggcaccagatacctgactcgtaatctgtaa

SEQ ID NO: 3625 - DNA, 2232 bp, synthetic construct
  source 1..2232: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcacaggctcgtgattctccgaagggttggcaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 3626 - DNA, 2232 bp, synthetic construct
  source 1..2232: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcacaggcttattctacggatgtgaggatgcaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgccgatcctccaacggccttcaacaaggacaagctgaactctttcatcacccagtattctactggccaagtcagcgtggagatcgagtgggagctgcagaaggaaaacagcaagcggtggaacccggagatccagtacacttccaactattacaagtctaataatgttgaatttgctgttaatactgaaggtgtatatagtgaaccccgccccattggcaccagatacctgactcgtaatctgtaa

SEQ ID NO: 3627 - DNA, 2232 bp, synthetic construct
  source 1..2232: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcacagattgttatgaattcgttgaaggctcaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgccgatcctccaacggccttcaacaaggacaagctgaactctttcatcacccagtattctactggccaagtcagcgtggagatcgagtgggagctgcagaaggaaaacagcaagcggtggaacccggagatccagtacacttccaactattacaagtctaataatgttgaatttgctgttaatactgaaggtgtatatagtgaaccccgccccattggcaccagatacctgactcgtaatctgtaa

SEQ ID NO: 3628 - DNA, 2232 bp, synthetic construct
  source 1..2232: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcacaggctcgggagagtcctcgtgggctgcaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgccgatcctccaacggccttcaacaaggacaagctgaactctttcatcacccagtattctactggccaagtcagcgtggagatcgagtgggagctgcagaaggaaaacagcaagcggtggaacccggagatccagtacacttccaactattacaagtctaataatgttgaatttgctgttaatactgaaggtgtatatagtgaaccccgccccattggcaccagatacctgactcgtaatctgtaa

SEQ ID NO: 3629 - DNA, 2232 bp, synthetic construct
  source 1..2232: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcacaggctagttttaatgatactagggctcaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgccgatcctccaacggccttcaacaaggacaagctgaactctttcatcacccagtattctactggccaagtcagcgtggagatcgagtgggagctgcagaaggaaaacagcaagcggtggaacccggagatccagtacacttccaactattacaagtctaataatgttgaatttgctgttaatactgaaggtgtatatagtgaaccccgccccattggcaccagatacctgactcgtaatctgtaa

SEQ ID NO: 3630 - DNA, 2232 bp, synthetic construct
  source 1..2232: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtggtggtacgttggccgtcgtgtcgcttgctcaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgccgatcctccaacggccttcaacaaggacaagctgaactctttcatcacccagtattctactggccaagtcagcgtggagatcgagtgggagctgcagaaggaaaacagcaagcggtggaacccggagatccagtacacttccaactattacaagtctaataatgttgaatttgctgttaatactgaaggtgtatatagtgaaccccgccccattggcaccagatacctgactcgtaatctgtaa

SEQ ID NO: 3631 - DNA, 2232 bp, synthetic construct
  source 1..2232: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcacaggcttatgggttgccgaagggtcctcaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgccgatcctccaacggccttcaacaaggacaagctgaactctttcatcacccagtattctactggccaagtcagcgtggagatcgagtgggagctgcagaaggaaaacagcaagcggtggaacccggagatccagtacacttccaactattacaagtctaataatgttgaatttgctgttaatactgaaggtgtatatagtgaaccccgccccattggcaccagatacctgactcgtaatctgtaa

SEQ ID NO: 3632 - DNA, 2232 bp, synthetic construct
  source 1..2232: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcacaggcttcgactgggacgcttcggcttcaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgccgatcctccaacggccttcaacaaggacaagctgaactctttcatcacccagtattctactggccaagtcagcgtggagatcgagtgggagctgcagaaggaaaacagcaagcggtggaacccggagatccagtacacttccaactattacaagtctaataatgttgaatttgctgttaatactgaaggtgtatatagtgaaccccgccccattggcaccagatacctgactcgtaatctgtaa

SEQ ID NO: 3633 - DNA, 2232 bp, synthetic construct
  source 1..2232: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcgcaggcgtattcgacggatgagaggatgcaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgccgatcctccaacggccttcaacaaggacaagctgaactctttcatcacccagtattctactggccaagtcagcgtggagatcgagtgggagctgcagaaggaaaacagcaagcggtggaacccggagatccagtacacttccaactattacaagtctaataatgttgaatttgctgttaatactgaaggtgtatatagtgaaccccgccccattggcaccagatacctgactcgtaatctgtaa

SEQ ID NO: 3634 - DNA, 2232 bp, synthetic construct
  source 1..2232: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcgcaggcgtattcgacggatgagaggaagcaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgccgatcctccaacggccttcaacaaggacaagctgaactctttcatcacccagtattctactggccaagtcagcgtggagatcgagtgggagctgcagaaggaaaacagcaagcggtggaacccggagatccagtacacttccaactattacaagtctaataatgttgaatttgctgttaatactgaaggtgtatatagtgaaccccgccccattggcaccagatacctgactcgtaatctgtaa

SEQ ID NO: 3635 - DNA, 2232 bp, synthetic construct
  source 1..2232: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcacaggcttatgtttcgtctgttaagatgcaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgccgatcctccaacggccttcaacaaggacaagctgaactctttcatcacccagtattctactggccaagtcagcgtggagatcgagtgggagctgcagaaggaaaacagcaagcggtggaacccggagatccagtacacttccaactattacaagtctaataatgttgaatttgctgttaatactgaaggtgtatatagtgaaccccgccccattggcaccagatacctgactcgtaatctgtaa

SEQ ID NO: 3636 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSPLNGAVHLYAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3637 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQARDSPKGWQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3638 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAYSTDVRMQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3639 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQIVMNSLKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3640 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQARESPRGLQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3641 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQASFNDTRAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3642 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSGGTLAVVSLAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3643 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAYGLPKGPQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3644 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQASTGTLRLQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3645 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAYSTDERMQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3646 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAYSTDERKQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3647 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAYVSSVKMQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3648 - protein, 9 aa, synthetic construct
PLNGAVHLY

SEQ ID NO: 3649 - protein, 7 aa, synthetic construct
RDSPKGW

SEQ ID NO: 3650 - protein, 7 aa, synthetic construct
YSTDVRM

SEQ ID NO: 3651 - protein, 7 aa, synthetic construct
IVMNSLK

SEQ ID NO: 3652 - protein, 7 aa, synthetic construct
RESPRGL

SEQ ID NO: 3653 - protein, 7 aa, synthetic construct
SFNDTRA

SEQ ID NO: 3654 - protein, 9 aa, synthetic construct
GGTLAVVSL

SEQ ID NO: 3655 - protein, 7 aa, synthetic construct
YGLPKGP

SEQ ID NO: 3656 - protein, 7 aa, synthetic construct
STGTLRL

SEQ ID NO: 3657 - protein, 7 aa, synthetic construct
YSTDERM

SEQ ID NO: 3658 - protein, 7 aa, synthetic construct
YSTDERK

SEQ ID NO: 3659 - protein, 7 aa, synthetic construct
YVSSVKM

SEQ ID NO: 3660 - DNA, 27 bp, synthetic construct
  source 1..27: mol_type = other DNA
ccgcttaatggtgccgtccatctttat

SEQ ID NO: 3661 - DNA, 23 bp, synthetic construct
  source 1..23: mol_type = other DNA
cgtgattctccgaagggttggca

SEQ ID NO: 3662 - DNA, 23 bp, synthetic construct
  source 1..23: mol_type = other DNA
tattctacggatgtgaggatgca

SEQ ID NO: 3663 - DNA, 23 bp, synthetic construct
  source 1..23: mol_type = other DNA
attgttatgaattcgttgaaggc

SEQ ID NO: 3664 - DNA, 23 bp, synthetic construct
  source 1..23: mol_type = other DNA
cgggagagtcctcgtgggctgca

SEQ ID NO: 3665 - DNA, 23 bp, synthetic construct
  source 1..23: mol_type = other DNA
agttttaatgatactagggctca

SEQ ID NO: 3666 - DNA, 27 bp, synthetic construct
  source 1..27: mol_type = other DNA
ggtggtacgttggccgtcgtgtcgctt

SEQ ID NO: 3667 - DNA, 21 bp, synthetic construct
  source 1..21: mol_type = other DNA
tatgggttgccgaagggtcct

SEQ ID NO: 3668 - DNA, 21 bp, synthetic construct
  source 1..21: mol_type = other DNA
tcgactgggacgcttcggctt

SEQ ID NO: 3669 - DNA, 21 bp, synthetic construct
  source 1..21: mol_type = other DNA
tattcgacggatgagaggatg

SEQ ID NO: 3670 - DNA, 21 bp, synthetic construct
  source 1..21: mol_type = other DNA
tattcgacggatgagaggaag

SEQ ID NO: 3671 - DNA, 21 bp, synthetic construct
  source 1..21: mol_type = other DNA
tatgtttcgtctgttaagatg

SEQ ID NO: 3672-4544 - skipped (873 entries)

SEQ ID NO: 4545 - protein, 4 aa, unidentified
KDEL

SEQ ID NO: 4546-4678 - skipped (133 entries)

SEQ ID NO: 4679 - DNA, 22 bp, unidentified
  source 1..22: mol_type = unassigned DNA
atacatacttctttacattcca

SEQ ID NO: 4680-5160 - skipped (481 entries)

SEQ ID NO: 5161 - DNA, 15 bp, synthetic construct
  source 1..15: mol_type = other DNA
ggaggaggaggaagt

SEQ ID NO: 5162 - DNA, 15 bp, synthetic construct
  source 1..15: mol_type = other DNA
ggaggtggaggttct

SEQ ID NO: 5163-5346 - skipped (184 entries)

SEQ ID NO: 5347 - DNA, 45 bp, synthetic construct
  source 1..45: mol_type = other DNA
ggtggtggtggatccggaggaggaggaagtggaggtggaggttct

SEQ ID NO: 5348 - protein, 15 aa, synthetic construct
GGGGSGGGGSGGGGS